SEQUENCE LISTING (3 sequences)

SEQ ID NO: 1 - DNA, 1500 bp, Homo sapiens
  source 1..1500: mol_type = other DNA
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatacgaccgaaaacctgtattttcagggcctgggatacggatcatgggaaattgatccaaaggacctgaccttcttgaaggagctggggactggacaatttggggtagtgaagtatgggaaatggagaggccagtacgacgtggccatcaagatgatcaaagaaggctccatgtctgaagatgaattcattgaagaagccaaagtcatgatgaatctttcccatgagaagctggtgcagttgtatggcgtctgcaccaagcagcgccccatcttcatcatcactgagtacatggccaatggctgcctcctgaactacctgagggagatgcgccaccgcttccagactcagcagctgctagagatgtgcaaggatgtctgtgaagccatggaatacctggagtcaaagcagttccttcaccgagacctggcagctcgaaactgtttggtaaacgatcaaggagttgttaaagtatctgatttcggcctgtccaggtatgtcctggatgatgaatacacaagctcagtaggctccaaatttccagtccggtggtccccaccggaagtcctgatgtatagcaagttcagcagcaaatctgacatttgggcttttggggttttgatgtgggaaatttactccctggggaagatgccatatgagagatttactaacagtgagactgctgaacacattgcccaaggcctacgtctctacaggcctcatctggcttcagagaaggtatataccatcatgtacagttgttggcatgagaaagcagatgagcgtcccactttcaaaattcttctgagcaatattctagatgtcatggatgaagaatcctga

SEQ ID NO: 2 - protein, 228 aa, Homo sapiens
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDTTENLYFQ

SEQ ID NO: 3 - protein, 271 aa, Homo sapiens
GLGYGSWEIDPKDLTFLKELGTGQFGVVKYGKWRGQYDVAIKMIKEGSMSEDEFIEEAKVMMNLSHEKLVQLYGVCTKQRPIFIITEYMANGCLLNYLREMRHRFQTQQLLEMCKDVCEAMEYLESKQFLHRDLAARNCLVNDQGVVKVSDFGLSRYVLDDEYTSSVGSKFPVRWSPPEVLMYSKFSSKSDIWAFGVLMWEIYSLGKMPYERFTNSETAEHIAQGLRLYRPHLASEKVYTIMYSCWHEKADERPTFKILLSNILDVMDEES

(19) United States
(12) Patent Application Publication
(10) Pub. No.: US 2023/0175038 A1
(43) Pub. Date: Jun. 8, 2023

(54) CRYSTAL STRUCTURE OF BTK PROTEIN AND BINDING POCKETS THEREOF

(71) Applicant: Janssen Pharmaceutica NV, Beerse (BE)

(72) Inventors: Richard S. ALEXANDER, Newark, DE (US); Cynthia MILLIGAN, Rutledge, PA (US); Mark S. TICHENOR, San Diego, CA (US)

(21) Appl. No.: 18/076,567

(22) Filed: Dec. 7, 2022

Related U.S. Application Data
(60) Provisional application No. 63/287,228, filed on Dec. 8, 2021.

(51) Int. Cl. C12Q 1/48; C07D 495/16

(52) U.S. Cl. CPC C12Q 1/485; C07D 495/16

(57) ABSTRACT

The present invention provides a crystal structure of human BTK with space group space group p 2 21 21 and has unit cell parameters a=38.155±2Å; b=72.394±2Å; c=103.946±2Å; a=90°; b=90°; g=90°; the BTK protein is complexed with N-((1R,2S)-2-Acrylamidocyclopentyl)-5-(S)-(6-isobutyl-4-methylpyridin-3-yl)-4-oxo-4,5-dihydro-3H-1-thia-3,5,8-triazaacenaphthylene-2-carboxamide.

### CROSS-REFERENCE TO OTHER APPLICATIONS

This application claims priority to U.S. Provisional Application No. 63/287,228 filed on Dec. 8, 2021 titled “CRYSTAL STRUCTURE OF BTK PROTEIN AND BINDING POCKETS THEREOF” which is incorporated herein by reference in its entirety.

### SEQUENCE LISTING

The contents of the electronic sequence listing (PRD4173USNP1 Sequence Listing.xml; Size: 5703 bytes; and Date of Creation: Dec. 5, 2022) is herein incorporated by reference in its entirety.

### FIELD OF THE INVENTION

The present disclosure is directed to the crystalline composition of human Bruton's tyrosine kinase (“BTK”) complexed with a ligand and the methods for identifying a candidate inhibitor of BTK. The present invention relates to BTK binding pockets. This invention also relates the methods of using the structure coordinates to solve the structure of homologous proteins or protein complexes. In addition, this invention relates to methods of using the structure coordinates to screen for and design compounds, including inhibitory compounds, that bind to BTK, or complexes thereof. The invention also relates to crystallizable compositions and crystals comprising BTK complexes with a compound of Formula (I):

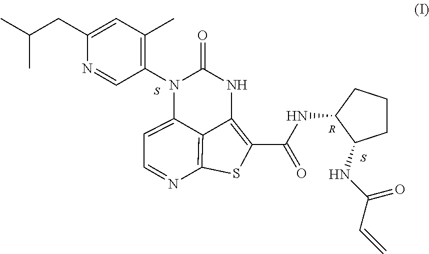

or pharmaceutically acceptable salts, hydrates, polymorphs or solvates thereof

### BACKGROUND OF THE INVENTION

Malignancies, in particular diffuse large B-cell lymphoma (DLBCL), mantle cell lymphoma (MCL), follicular lymphoma (FL), marginal zone lymphoma (MZL), chronic lymphocytic leukemia (CLL), small lymphocytic lymphoma (SLL), Waldenström macroglobulinemia, and other conditions such as chronic graft versus host disease, continues to afflict patients. Alternative, effective treatments of cancer are still needed.

BTK is a ˜76 kDa protein belonging to the Tec family of non-receptor tyrosine kinases. Tec kinases form the second largest family of cytoplasmic tyrosine kinases in mammalian cells, which consists of four other members in addition to BTK: the eponymous kinase TEC, ITK, TXK/RLK and BMX. Tec kinases are evolutionarily conserved throughout vertebrates. They are related to, but structurally distinct from, the larger Src and Syk kinase families. Tec family proteins are abundantly expressed in hematopoietic tissues and play important roles in the growth and differentiation of blood and endothelial cells in mammals.

Based upon BTK expression from IHC studies described in the art, Btk inhibition has the potential to modulate biology associated with B cells, macrophages, mast cells, osteoclasts, and platelet microparticles. Corneth, O. B., et al. Curr. Top. Microbiol. Immunol. BTK Signaling in B Cell Differentiation and Autoimmunity. 2015 Sep. 5.

Accordingly, there has been an interest in finding inhibitors of BTK that can serve as effective therapeutic agents. New BTK ligand bound crystal structures reveal additional hydrogen bonding opportunities. Understanding the scope of the binding pocket flexibility and the limitations thereof is crucial to the design of new BTK inhibitors.

Thus, there remains a need for novel structure coordinates of ligand bound BTK protein to screen for and design new ligands, including inhibitory ligands, which may provide a therapeutic benefit to patients suffering from cancer and/or immunological diseases. The present invention addresses this need by providing compounds and pharmaceutical compositions thereof that are effective as BTK inhibitors. Applicants have also addressed this need by providing the crystal structure of BTK and BTK-inhibitor complexes. Solving these crystal structures has allowed the determination of the key structural features of BTK, particularly the shape of its binding pockets.

### SUMMARY OF THE INVENTION

One aspect of the present invention is a crystalline composition comprising amino acid residues of SEQ ID NO:3, and a ligand, wherein said crystalline composition is characterized with space group p 2 21 21 and has unit cell parameters a=38.155±2Å; b=72.394±2Å; c=103.946±2Å; a=90°; b=90°; g=90°. In some embodiments, the ligand is a compound of Formula (I):

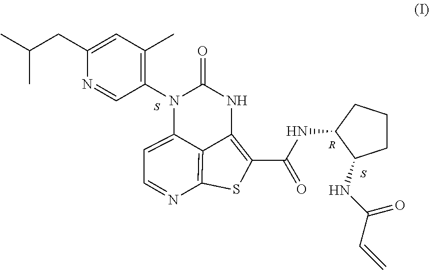

or pharmaceutically acceptable salts, hydrates, polymorphs or solvates thereof.

Another aspect of the present invention are methods for identifying candidate inhibitors of BTK, the method comprising: generating a three-dimensional structure of a binding site of BTK on a computer, wherein the three dimensional structure comprises the coordinates of the unit cell and space group parameters of the crystalline composition of SEQ ID NO: 3, employing said three dimensional structure to design or select a candidate inhibitor; and contacting said candidate inhibitor with human BTK and measuring the ability of said candidate inhibitor to bind to BTK.

A further aspect of the present invention are methods for identifying and/or designing a candidate inhibitor using a BTK crystal comprising a BTK protein, wherein said method comprises: a) preparing the crystalline composition of SEQ ID NO: 3 and a ligand, wherein the ligand is a compound of Formula (I):

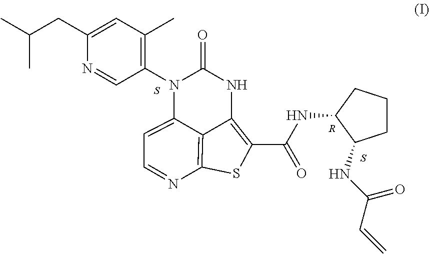

or pharmaceutically acceptable salts, hydrates, polymorphs or solvates thereof; b) soaking another candidate inhibitor into the crystalline composition, displacing the compound of Formula (I) (original ligand) and forming an inhibitor-crystal complex; c) determining the three-dimensional structure coordinates of the inhibitor-crystal complex prepared in step b); and d) using the structure coordinates from step c) to design or identify a candidate inhibitor; and e) contacting said candidate inhibitor with human BTK and measuring the ability of said candidate inhibitor to bind to BTK.

The invention also provides crystallizable compositions and crystal compositions comprising BTK with or without a chemical entity. The invention also provides a method for crystallizing a BTK protein or a BTK protein complex.

The invention also provides a data storage medium which comprises the structure coordinates of molecules or molecular complexes of the BTK binding pockets. In one embodiment, the data storage medium comprises the structure coordinates of the binding pocket. The invention also provides a computer comprising the data storage medium. Such storage medium when read and utilized by a computer programmed with appropriate software can display, on a computer screen or similar viewing device, a three-dimensional graphical representation of such binding pockets.

The invention also provides methods for designing, evaluating and identifying compounds which bind to the molecules or molecular complexes or their binding pockets. Such compounds are potential inhibitors of BTK or its homologues.

The invention also provides a method for determining at least a portion of the three-dimensional structure of molecules or molecular complexes which contain at least some structurally similar features to BTK. This is achieved by using at least some of the structure coordinates obtained from the BTK protein or protein complexes.

Some aspects of the invention are directed to a crystalline composition comprising SEQ ID NO:3, and a ligand, wherein said crystalline composition is characterized with space group p 2 21 21 and has unit cell parameters a=38.155±2Å; b=72.394±2Å; c=103.946±2Å; a=90°; b=90°; g=90°. In some aspects, the ligand is a compound of Formula (I):

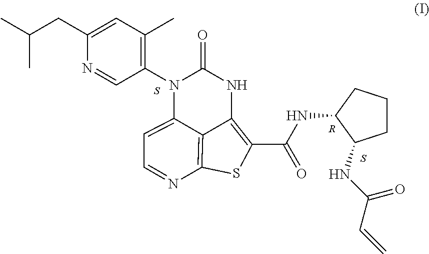

or pharmaceutically acceptable salts, hydrates, polymorphs or solvates thereof

Some aspects are directed to methods for identifying a candidate inhibitor of BTK, wherein said method comprises: generating a three-dimensional structure of a binding site of BTK on a computer, wherein the three dimensional structure coordinates possess the unit cell and space group parameters of the crystalline composition comprising SEQ ID NO:3, and a compound of Formula (I):

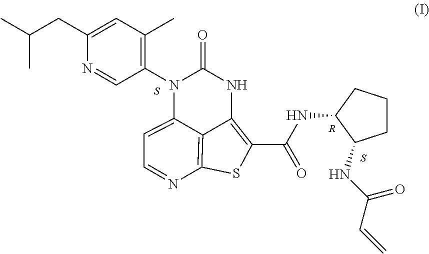

or pharmaceutically acceptable salts, hydrates, polymorphs or solvates thereof, employing said three dimensional structure to design or select a candidate inhibitor; and contacting said candidate inhibitor with human BTK and measuring the ability of said candidate inhibitor to bind to BTK. In some aspects, the candidate inhibitor makes a hydrogen bond with Asp539. In some aspects, the candidate inhibitor makes a hydrogen bond with Lys 430. In some aspects, the candidate inhibitor makes a hydrogen bond with Met477. In some aspects, the candidate inhibitor makes hydrogen bonds with Asp539, Lys 430, and Met477.

Some aspects are directed to methods for identifying and/or designing a candidate inhibitor using a human BTK crystal comprising a human BTK protein, wherein said method comprises:
a) preparing the crystalline composition of comprising SEQ ID NO:3, and compound I and b) soaking another candidate inhibitor into the crystalline composition, displacing the compound of Formula (I) (original ligand) to form an inhibitor-crystal complex; c) determining the three-dimensional structure coordinates of the inhibitor-crystal complex prepared in step b); d) using the structure coordinates from step c) to design and/or identify a candidate inhibitor; and e) contacting said candidate inhibitor with human BTK and measuring the ability of said candidate inhibitor to bind to BTK.

Some aspects are directed to methods of designing a compound or complex that interacts with a binding pocket or domain selected from the group comprising (i) BTK amino acid residues Leu408, Gly409, Thr410, Gly411, Va1416, Ala428, Lys430, Asn439, Met449, Leu452, Va1458, Ile472, Thr474, Glu475, Tyr476, Met477, Gly480, Cys481, Asn 484, Arg525, Leu528, Ser538, Asp539, and Phe540, according to Table 2; and (ii) BTK amino acid residues according to Table 2; comprising the steps of:
(a) producing a crystal of BTK in complex with a compound of Formula (I):

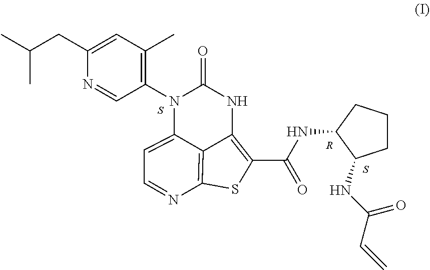

or pharmaceutically acceptable salts, hydrates, polymorphs or solvates thereof, wherein said BTK comprises SEQ ID NO: 3, wherein said crystal is characterized with space group space group p 2 21 21 and has unit cell parameters a=38.155±2Å; b=72.394±2Å; c=103.946±2Å; a=90°; b=90°; g=90°;
(b) providing the structure coordinates of said binding pocket or domain of the crystal of step (a), according to Table 2, on a computer capable of generating three-dimensional structural information from said structure coordinates, wherein the root mean square deviation of the backbone atoms is not greater than about 2.5 Å;
(c) using the computer to dock a first chemical entity in part of the binding pocket or domain;
(d) docking at least a second chemical entity in another part of the binding pocket or domain;
(e) quantifying the association between the first or second chemical entity and part of the binding pocket or domain;
(f) repeating steps (c) to (e) with a t least one additional chemical entity, selecting the at least one additional chemical entity based on said quantified association of all of said first, second, and at least one additional chemical entity;
(g) optionally, visually inspecting the relationship of the first, second and at least one additional chemical entity to each other in relation to the binding pocket or domain on a computer screen using the three-dimensional graphical representation of the binding pocket or domain and said first, second and at least one additional chemical entity; and
(h) assembling the first, second and at least one additional chemical entity into a compound or complex that interacts with said binding pocket or domain by model building.

Some aspects are directed to methods for identifying a candidate inhibitor that interacts with a binding site of a BTK protein, comprising the steps of:
(a) producing a crystal of BTK in complex with a compound of Formula (I):

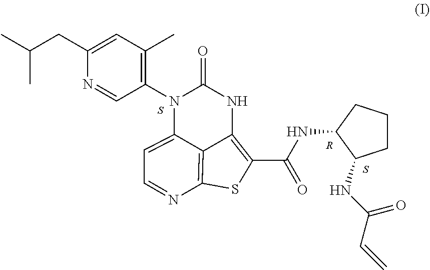

or pharmaceutically acceptable salts, hydrates, polymorphs or solvates thereof, wherein said BTK comprises SEQ ID NO: 3, wherein the crystal is characterized with space group p 2 21 21 and has unit cell parameters a=38.155±2Å; b=72.394±2Å; c=103.946±2Å; a=90°; b=90°; g=90°;
(b) obtaining the structure coordinates of amino acids of the crystal of step (a) according to Table 2;
(c) generating a three-dimensional model of said BTK protein using the structure coordinates of the amino acids generated in step (b), wherein the root mean square deviation from backbone atoms is not more than ±2.0 Å;
(d) determining a binding site of said human BTK protein from said three-dimensional model; and
(e) performing computer fitting analysis to identify the candidate inhibitor which interacts with said binding site.

Some aspects further comprise the step of: (f) contacting the identified candidate inhibitor with said BTK protein in order to determine the effect of the inhibitor on BTK activity.

In some aspects, the binding site of said BTK protein determined in step (d) comprises the structure coordinates, according to Table 2, of BTK amino acid residues Leu408, Gly409, Thr410, Gly411, Va1416, Ala428, Lys430, Asn439, Met449, Leu452, Va1458, Ile472, Thr474, Glu475, Tyr476, Met477, Gly480, Cys481, Asn 484, Arg525, Leu528, Ser538, Asp539, and Phe540, wherein the root mean square deviation is not more than ±2.0 Å.

Some aspects are directed to methods of using a crystal comprising a BTK binding pocket or domain in an inhibitor screening assay comprising the steps of:
(a) producing a crystal of BTK in complex with a compound of Formula (I):

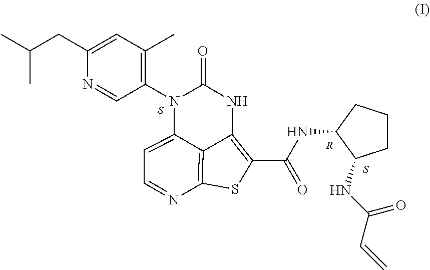

or pharmaceutically acceptable salts, hydrates, polymorphs or solvates thereof, wherein said BTK binding pocket comprises SEQ ID NO: 3, wherein said crystal is characterized with space group p 2 21 21 and has unit cell parameters a=38.155±2Å; b=72.394±2Å; c=103.946±2Å; a=90°; b=90°; g=90°;
(b) providing the structure coordinates of said binding pocket or domain of the crystal of step (a), according to Table 2, on a computer capable of generating three-dimensional structural information from said structure coordinates;
(c) selecting a potential inhibitor by performing rational drug design with a three-dimensional structure determined for the crystal, wherein said selecting is performed in conjunction with computer modeling;
(d) contacting the potential inhibitor with the kinase; and
(e) detecting the ability of the potential inhibitor for inhibiting the kinase's enzymatic activity.

Some aspects are drawn to methods of designing a compound or complex that interacts with a BTK binding pocket or domain comprising the steps of:
(a) producing a crystal of human BTK in complex with a compound of Formula (I):

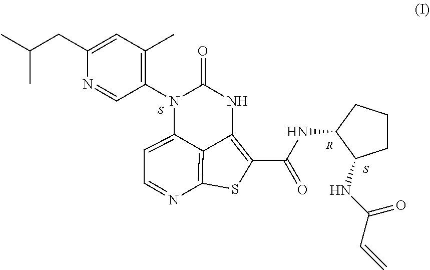

or pharmaceutically acceptable salts, hydrates, polymorphs or solvates thereof, wherein said BTK comprises SEQ ID NO^{.} 3, wherein said crystal is characterized with space group p 2 21 21 and has unit cell parameters a=38.155±2Å; b=72.394±2Å; c=103.946±2Å; a=90°; b=90°; g=90°;
(b) providing the structure coordinates of said binding pocket or domain of the crystal of step (a), according to Table 2, on a computer capable of generating three-dimensional structural information from said structure coordinates;
(c) using the computer to dock a first chemical entity in part of the binding pocket or domain;
(d) docking at least a second chemical entity in another part of the binding pocket or domain;
(e) quantifying the association between the first or second chemical entity and part of the binding pocket or domain;
(f) repeating steps (c) to (e) with at least one additional chemical entity, selecting a first, second and at least one additional chemical entity based on said quantified association of all of said first, second and at least one additional chemical entity;
(g) optionally, visually inspecting the relationship of the first, second and at least one additional chemical entity to each other in relation to the binding pocket or domain on a computer screen using the three-dimensional graphical representation of the binding pocket or domain and said first, second and at least one additional chemical entity; and
(h) assembling the first, second and at least one additional chemical entity into a compound or complex that interacts with said binding pocket or domain by model building.

Some aspects are directed to methods for identifying a candidate inhibitor that interacts with a binding site of a BTK protein comprising the steps of:
(a) producing a crystal of BTK in complex with a compound of Formula (I):

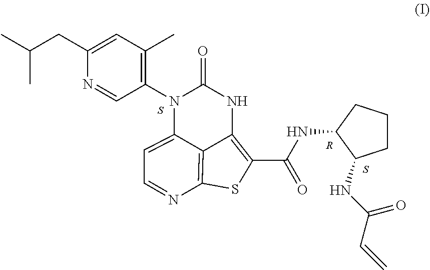

or pharmaceutically acceptable salts, hydrates, polymorphs or solvates thereof, wherein said BTK comprises SEQ ID NO: 3, wherein said crystal is characterized with space group p 2 21 21 and has unit cell parameters a=38.155±2Å; b=72.394±2Å; c=103.946±2Å; a=90°; b=90°; g=90°;
(b) determining the three-dimensional structure coordinates of BTK using the crystal in step (a);
(c) using the structure coordinates from step (b) to generate a three-dimensional structure of the crystallized BTK protein produced in step (a);
(d) employing said three-dimensional structure to design or select said candidate inhibitor;
(e) synthesizing said candidate inhibitor; and
(f) contacting said candidate inhibitor with human BTK protein to determine the ability of said candidate inhibitor to bind to human BTK.

Some aspects are directed to methods for identifying a candidate inhibitor that interacts with a binding site of a human BTK protein comprising the steps of:
(a) producing a crystal of BTK in complex with a compound of Formula (I):

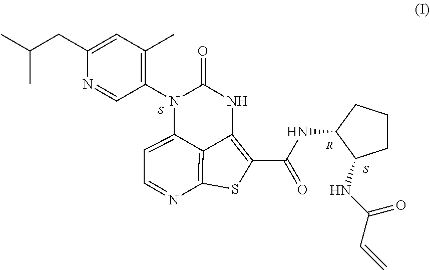

or pharmaceutically acceptable salts, hydrates, polymorphs or solvates thereof, wherein said BTK comprises SEQ ID NO: 3, wherein said crystal is characterized with space group p 2 21 21 and has unit cell parameters a=38.155±2Å; b=72.394±2Å; c=103.946±2Å; a=90°; b=90°; g=90°;
(b) determining the three-dimensional structure coordinates of BTK using the crystal in step (a);
(c) using the structure coordinates from step (b) to generate a three-dimensional structure of a molecular complex comprising a binding site of amino acid residues Leu408, Gly409, Thr410, Gly411, Va1416, Ala428, Lys430, Asn439, Met449, Leu452, Va1458, Ile472, Thr474, Glu475, Tyr476, Met477, Gly480, Cys481, Asn 484, Arg525, Leu528, Ser538, Asp539, and Phe540 according to Table 2, wherein the root mean square deviation of the backbone atoms is not greater than about 2.5 Å;
(d) employing said three-dimensional structure to design or select said candidate inhibitor;
(e) synthesizing said candidate inhibitor; and
(f) contacting said candidate inhibitor with BTK to determine the ability of said candidate inhibitor to bind to BTK.

### BRIEF DESCRIPTION OF THE DRAWINGS

The accompanying drawings, which are incorporated in and form a part of the specification, illustrate the embodiments of the application and together with the written description serve to explain the principles, characteristics, and features of the invention. In the drawings:

FIG. 1 depicts the chemical structure of Compound I.

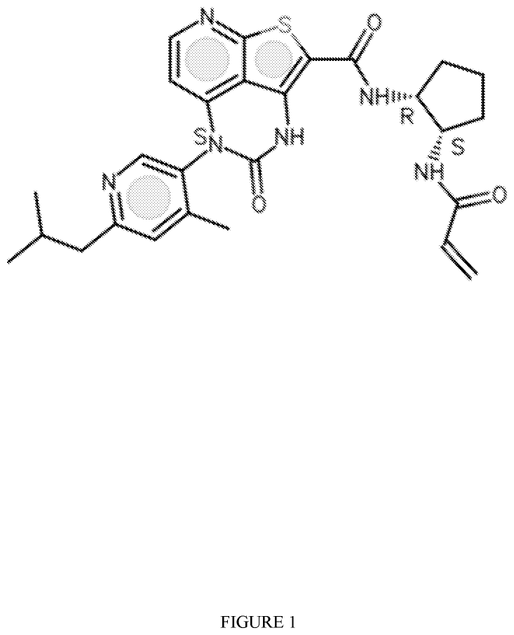

FIG. 2 depicts the electron density of Compound I when in complex with BTK. Density does cover the entire compound with clear density observed for the covalent portion of the interaction including a direct attachment to Cys481. Light: 2fₒ-f_{c} map at 1σ contour level (shows density for modeled atoms and unmodeled or missing atoms, Shows no density for clearly superfluous atoms. Dark Map: level. fₒ-f_{c} at +/−3.5σ level (shows positive density for unmodeled or missing atoms; shows negative density for superfluous atoms.

FIG. 3 depicts the overall structure of the BTK-Compound I complex. Compound I is represented as a stick diagram and BTK protein as a cartoon diagram. The BTK-Compound I complex adopts a bilobal architecture characteristic. Compound I binds to the ATP binding site and neighboring regions of the active site in the cleft formed between the N-terminal and C-terminal lobe of BTK.

FIG. 4 depicts interactions between BTK and Compound I in the active site. A direct attachment between Compound I and BTK occurs through a bond with the sulfur atom of Cys481. Hydrogen bonds (dashed lines) are observed between Compound I and residues Asp539, Lys430, and Met477. Solvent molecules are also observed in hydrogen bonds with Compound I.

FIG. 5 depicts detailed interaction between BTK and Compound I. The following residues can be found in the vicinity of the ligand with a maximum distance of 4.0 Å; Leu408, Gly409, Thr410, Gly411, Va1416, Ala428, Lys430, Asn439, Met449, Leu452, Va1458, Ile472, Thr474, Glu475, Tyr476, Met477, Gly480, Cys481, Asn 484, Arg525, Leu528, Ser538, Asp539, and Phe540.

### DETAILED DESCRIPTION OF THE INVENTION

The disclosure may be more fully appreciated by reference to the following description, including the following glossary of terms and the concluding examples. It is to be appreciated that certain features of the disclosed compositions and methods which are, for clarity, described herein in the context of separate aspects, may also be provided in combination in a single aspect. Conversely, various features of the disclosed compositions and methods that are, for brevity, described in the context of a single aspect, may also be provided separately or in any subcombination. It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to aspects containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (for example, “ a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed aspect.

The following abbreviations are used throughout the application:

| | | | | | |
|---|---|---|---|---|---|
| A= | Ala= | Alanine | T= | Thr= | Threonine |
| V= | Val= | Valine | C= | Cys= | Cysteine |
| L= | Leu= | Leucine | Y= | Tyr= | Tyrosine |
| I= | Ile= | Isoleucine | N= | Asn= | Asparagine |
| P= | Pro= | Proline | Q= | Gln= | Glutamine |
| F= | Phe= | Phenylalanine | D= | Asp= | Aspartic Acid |
| W= | Trp= | Tryptophan | E= | Glu= | Glutamic Acid |
| M= | Met= | Methionine | K= | Lys= | Lysine |
| G= | Gly= | Glycine | R= | Arg= | Arginine |
| S= | Ser= | Serine | H= | His= | Histidine |

The term “about” as used herein in the context of RMSD values takes into consideration the standard error of the RMSD value, which is ±0.1 Å. When “about” is used immediately preceding a numerical value means a range of plus or minus 10% of that value, for example, “about 50” means 45 to 55, “about 25,000” means 22,500 to 27,500, etc., unless the context of the disclosure indicates otherwise, or is inconsistent with such an interpretation.

The term “associating with” as used herein refers to a condition of proximity between a chemical entity or compound, or portions thereof, and a binding pocket or binding site on a protein. The association may be non-covalent - wherein the juxtaposition is energetically favored by hydrogen bonding or van der Waals or electrostatic interactions - or it may be covalent.

The term “binding pocket” as used herein refers to a region of a molecule or molecular complex, that, as a result of its shape and charge, favorably associates with another chemical entity or compound. The term “pocket” includes, but is not limited to, cleft, channel or site. BTK or BTK-like molecules may have binding pockets which include, but are not limited to, peptide or substrate binding, and antibody binding sites. The binding pocket may also mean the ATP binding domain and neighboring regions of BTK located at amino acid residues 389-659. The binding pocket may also mean the ATP binding domain and neighboring regions of BTK represented by SEQ ID NO.3.

The term “chemical entity” as used herein refers to chemical compounds, complexes of at least two chemical compounds, and fragments of such compounds or complexes. The chemical entity may be, for example, a ligand, a substrate, a nucleotide triphosphate, a nucleotide diphosphate, phosphate, a nucleotide, an agonist, antagonist, inhibitor, antibody, drug, peptide, protein or compound.

The term “compound” or “compounds” and equivalent expressions are used herein to mean all compounds described herein, and in particular a compound of Formula (I):

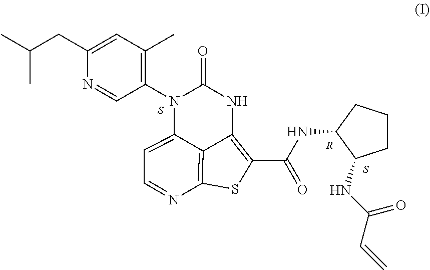

or pharmaceutically acceptable salts, hydrates, polymorphs or solvates thereof where the context so permits. Similarly, reference to intermediates, whether or not they themselves are claimed, is meant to embrace their salts, and solvates, where the context so permits. The compound of Formula (III) is also known as N-((1R,2S)-2-Acrylamidocyclopentyl)-5-(S)-(6-isobutyl-4-methylpyridin-3-yl)-4-oxo-4,5-dihydro-3H-1-thia-3,5,8-triazaacenaphthylene-2-carboxamide .

The term “comprise”, or variations such as “comprises” or “comprising” as used herein will be understood to imply the inclusion of a stated integer or groups of integers but not exclusion of any other integer or groups of integers.

The term “consisting essentially of” means the method or composition includes the steps or components specifically recited, and may also include those that do not materially affect the basic and novel characteristics of the present invention.

The term “consisting of” means the method or composition includes only the steps or components specifically recited. It must be noted that, as used herein, and in the appended claims, the singular forms “a”, “an” and “the” include plural reference unless the context clearly dictates otherwise.

The term “corresponding amino acid” or “residue which corresponds to” as used herein refers to a particular amino acid or analogue thereof in a BTK protein that is identical or functionally equivalent to an amino acid in BTK according to SEQ ID NO: 3.

Methods for identifying a corresponding amino acid are known in the art and are based upon sequence, structural alignment, its functional position or a combination thereof as compared to BTK. For example, corresponding amino acids may be identified by superimposing the back bone atoms of the amino acids in BTK using well known software applications, such as QUANTA (Accelrys, San Diego, Calif. ©2001, 2002). The corresponding amino acids may also be identified using sequence alignment programs such as the “bestfit” program available from the Genetics Computer Group which uses the local homology algorithm described by Smith and Waterman in Advances in Applied Mathematics 2, 482 (1981), which is incorporated herein by reference.

The term “crystallization solution” as used herein refers to a solution which promotes crystallization comprising at least one agent including a buffer, one or more salts, a precipitating agent, one or more detergents, sugars or organic compounds, lanthanide ions, a poly-ionic compound, and/or stabilizer.

The term “domain” as used herein refers to a portion of the BTK protein that can be separated based on its biological function, for example, catalysis. The domain may comprise a binding pocket, a sequence or a structural motif.

The term “fitting operation” as used herein refers to an operation that utilizes the structure coordinates of a chemical entity, binding pocket, molecule or molecular complex, or portion thereof, to associate the chemical entity with the binding pocket, molecule or molecular complex, or portion thereof. This may be achieved by positioning, rotating or translating the chemical entity in the binding pocket to match the shape and electrostatic complementarity of the binding pocket. Covalent interactions, non-covalent interactions such as hydrogen bond, electrostatic, hydrophobic, van der Waals interactions, and non-complementary electrostatic interactions such as repulsive charge-charge, dipole-dipole and charge-dipole interactions may be optimized. Alternatively, one may minimize the deformation energy of binding of the chemical entity to the binding pocket.

The term “generating a three-dimensional structure” or “generating a three-dimensional representation” as used herein refers to converting the lists of structure coordinates into structural models or graphical representation in three-dimensional space. This can be achieved through commercially or publicly available software. The three-dimensional structure may be displayed or used to perform computer modeling or fitting operations. In addition, the structure coordinates themselves may be used to perform computer modeling and fitting operations.

The term “BTK protein” as used herein refers to the human BTK protein encoded by the BTK gene. In some aspects, the term “BTK protein” as used herein refers to the ATP binding domain and neighboring regions of BTK located at amino acid residues 389-659 of BTK. In some aspects, the term “BTK protein” as used herein refers to the BTK kinase domain protein which is represented by SEQ ID NO.3.

The term “molecular complex” or “complex” as used herein in the singular or plural refers to a molecule associated with at least one chemical entity.

The term “motif” as used herein refers to a portion of the BTK protein that defines a structural compartment or carries out a function in the protein, for example, catalysis, structural stabilization, or phosphorylation. The motif may be conserved in sequence, structure and function. The motif can be contiguous in primary sequence or three-dimensional space. Examples of a motif include but are not limited to the protease domain and binding site.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

The term “pharmaceutically acceptable salt” as used herein refers to a salt of a compound of the disclosure that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like.

The term “root mean square deviation” or “RMSD” as used herein means the square root of the arithmetic mean of the squares of the deviations from the mean. It is a way to express the deviation or variation from a trend or object. For purposes of this invention, the “root mean square deviation” defines the variation in the backbone of a protein from the backbone of BTK, a binding pocket, a motif, a domain, or portion thereof, as defined by the structure coordinates of BTK described herein. It would be apparent to the skilled worker that the calculation of RMSD involves a standard error.

The term “soaked” as used herein refers to a process in which the crystal is transferred to a solution containing a compound of interest.

The term “structure coordinates” as used herein refers to Cartesian coordinates derived from mathematical equations related to the patterns obtained on diffraction of a monochromatic beam of X-rays by the atoms (scattering centers) of a protein or protein complex in crystal form. The diffraction data are used to calculate an electron density map of the repeating unit of the crystal. The electron density maps are then used to establish the positions of the individual atoms of the molecule or molecular complex.

The term “three-dimensional structural information” as used herein refers to information obtained from the structure coordinates. Structural information generated can include the three-dimensional structure or graphical representation of the structure. Structural information can also be generated when subtracting distances between atoms in the structure coordinates, calculating chemical energies for a BTK molecule or molecular complex or homologs thereof, calculating or minimizing energies for an association of a BTK molecule or molecular complex or homologs thereof to a chemical entity.

The terms “treating” or “treatment” of any disease or disorder as used herein refers, in one aspect, to ameliorating the disease or disorder (i.e., arresting or reducing the development of the disease or at least one of the clinical symptoms thereof). In another aspect “treating” or “treatment” refers to ameliorating at least one physical parameter, which may not be discernible by the subject. In yet another aspect, “treating” or “treatment” refers to modulating the disease or disorder, either physically, (e.g., stabilization of a discernible symptom), physiologically, (e.g., stabilization of a physical parameter), or both. In yet another aspect, “treating” or “treatment” refers to delaying the onset of the disease or disorder.

### Crystallizable Compositions and Crystals of BTK and Complexes Thereof

According to one aspect, the invention provides a crystal or crystallizable composition comprising BTK kinase domain or a BTK kinase domain complexed with a ligand. In one aspect, the ligand is a compound of Formula (I):

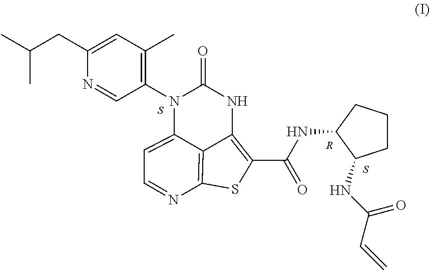

or pharmaceutically acceptable salts, hydrates, polymorphs or solvates thereof

The BTK kinase domain in the crystal or crystallizable composition comprises SEQ ID NO:3. In another aspect, BTK kinase domain comprises amino acid residues 389-659 of the BTK protein.

One aspect is a crystalline composition comprising SEQ ID NO:3, and a ligand, wherein said crystalline composition is characterized with space group p 2 21 21 and has unit cell parameters a=38.155±2Å; b=72.394±2Å; c=103.946±2Å; a=90°; b=90°; g=90°. In some aspects the ligand is a compound of Formula (I):

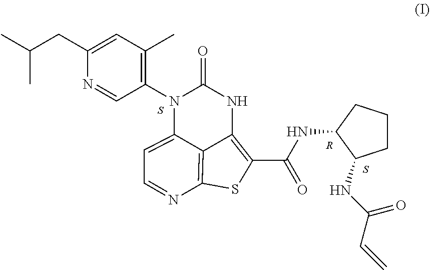

or pharmaceutically acceptable salts, hydrates, polymorphs or solvates thereof

One aspect is a crystalline composition comprising BTK complexed with a ligand. The present disclosure provides a crystalline composition comprising amino acid residues 389-659 of BTK, and a ligand, wherein said crystalline composition is characterized with space group p 2 21 21 and has unit cell parameters a=38.155±2Å; b=72.394±2Å; c=103.946±2Å; a=90°; b=90°; g=90°. In some aspects, the ligand is a compound of Formula (I):

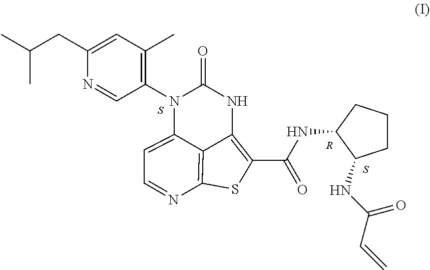

or pharmaceutically acceptable salts, hydrates, polymorphs or solvates thereof

According to one aspect, the invention provides a crystal or crystallizable composition consisting essentially of the BTK kinase domain or a BTK kinase domain complexed with a ligand. In one aspect, the ligand is a compound of Formula (I):

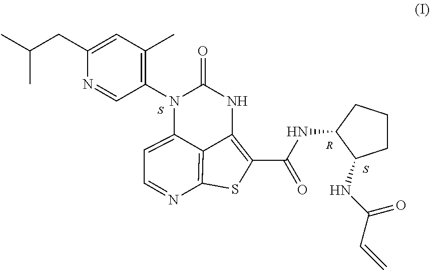

or pharmaceutically acceptable salts, hydrates, polymorphs or solvates thereof

The BTK kinase domain in the crystal or crystallizable composition consists essentially of SEQ ID NO:3. In another aspect, BTK kinase domain consists essentially of amino acid residues 389-659of the BTK protein.

One aspect is a crystalline composition consisting essentially of SEQ ID NO:3, and a ligand, wherein said crystalline composition is characterized with space group p 2 21 21 and has unit cell parameters a=38.155±2Å; b=72.394±2Å; c=103.946±2Å; a=90°; b=90°; g=90°. In some aspects a compound of Formula (I):

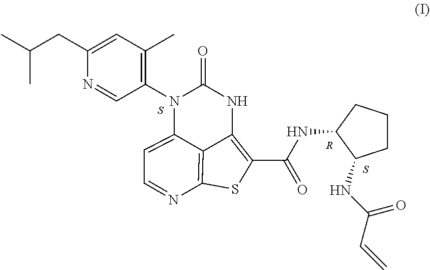

or pharmaceutically acceptable salts, hydrates, polymorphs or solvates thereof

One aspect is a crystalline composition consisting essentially of BTK complexed with a ligand. The present disclosure provides a crystalline composition consisting essentially of amino acid residues 389-659 of BTK, and a ligand, wherein said crystalline composition is characterized with space group p 2 21 21 and has unit cell parameters a=38.155±2Å; b=72.394±2Å; c=103.946±2Å; a=90°; b=90°; g=90°. In some aspects, the ligand is a compound of Formula (I):

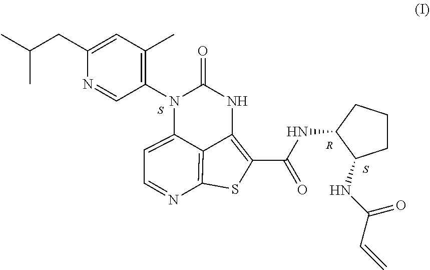

or pharmaceutically acceptable salts, hydrates, polymorphs or solvates thereof

According to one aspect, the invention provides a crystal or crystallizable composition consisting of BTK kinase domain a BTK kinase domain complexed with a ligand. In one aspect, the ligand is a compound of Formula (I):

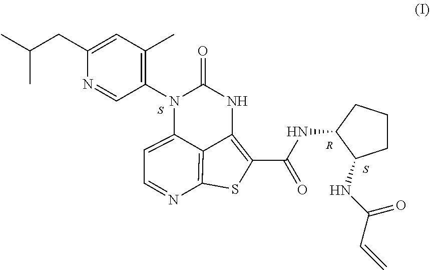

or pharmaceutically acceptable salts, hydrates, polymorphs or solvates thereof

The BTK kinase domain in the crystal or crystallizable composition consists of SEQ ID NO:3. In another aspect, the BTK kinase domain consists essentially of amino acid residues 389-659 of the BTK protein.

One aspect is a crystalline composition consisting of SEQ ID NO:3, and a ligand, wherein said crystalline composition is characterized with space group p 2 21 21 and has unit cell parameters a=38.155±2Å; b=72.394±2Å; c=103.946±2Å; a=90°; b=90°; g=90°. In some aspects the ligand is a compound of Formula (I):

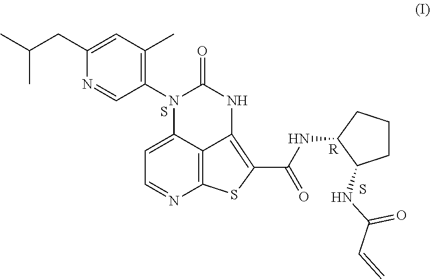

or pharmaceutically acceptable salts, hydrates, polymorphs or solvates thereof

One aspect is a crystalline composition consisting of BTK complexed with a ligand. The present disclosure provides a crystalline composition consisting of amino acid residues 389-659 of BTK, and a ligand, wherein said crystalline composition is characterized with space group p 2 21 21 and has unit cell parameters a=38.155±2Å; b=72.394±2Å; c=103.946±2Å; a=90°; b=90°; g=90°. In some aspects, the ligand is a compound of Formula (I):

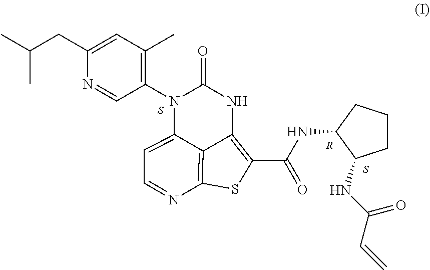

or pharmaceutically acceptable salts, hydrates, polymorphs or solvates thereof

The crystal structure of BTK in complex with Compound I bound to a binding site provides important structural information for the development of novel BTK inhibitors. The invention comprises a crystalline composition comprising SEQ ID NO:3, and Compound I, wherein said crystalline composition is characterized with space group p 2 21 21 and has unit cell parameters a=38.155±2Å; b=72.394±2Å; c=103.946±2Å; a=90°; b=90°; g=90°. The invention comprises a crystalline composition consisting essentially of SEQ ID NO:3, and Compound I, wherein said crystalline composition is characterized with space group p 2 21 21 and has unit cell parameters a=38.155±2Å; b=72.394±2Å; c=103.946±2Å; a=90°; b=90°; g=90°. The invention comprises a crystalline composition consisting of SEQ ID NO:3, and Compound I, wherein said crystalline composition is characterized with space group p 2 21 21 and has unit cell parameters a=38.155±2Å; b=72.394±2Å; c=103.946±2Å; a=90°; b=90°; g=90°.

Persons skilled in the art will recognize that modification at C terminus and N terminus is possible, as well as some amino acid residue mutations, substitutions, including glycosylations, acylations, and methylations. All such modifications are considered to be equivalents within the scope of the invention.

One aspect is a crystalline composition or crystal comprising BTK in the presence or absence of a chemical entity. Preferably, the chemical entity is a compound of Formula (I):

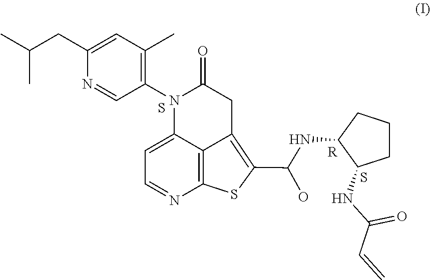

or pharmaceutically acceptable salts, hydrates, polymorphs or solvates thereof

It will be readily apparent to those skilled in the art that the unit cells of the crystal compositions may deviate ±1-2 Å from the above cell dimensions depending on the deviation in the unit cell calculations.

In one embodiment, the crystallizable composition comprises a crystallization solution of the BTK protein, a salt, a buffer between pH 5.0 and 7.0, 0-10 mM DTT and a polyethylene glycol. The salt includes, but is not limited to, KCl, LiSO4, NaCl and (NH₄)₂SO₄. The polyethylene glycol includes, but is not limited to, PEG3350, PEGMME 550, PEGMME2000, PEG4000, PEG6000. If the crystals are derived from seeding techniques, the concentrations of the polyethylene glycol may be less than 35%. In another aspect, the crystallizable composition comprises a crystallization solution of equal volumes of the BTK protein (8 mg/ml in 20 mM Tris pH 8, 150 mM NaCl, 2 mM DTT and a solution of 25-35% PEG MME 5K, 0.1M MES pH 6-7, 0.1-0.2M AmSO4.

Crystals can be grown using sitting drop or hanging drop vapor diffusion techniques, such as, but not limited to techniques described herein. Crystals can be grown in the Corning® 384 Well plate (available from Fisher Scientific), Greiner crystallization low profile plates (available from Hampton Research (Aliso Viejo, Calif.)), both the 96-well CrystalQuick™ standard profile round and flat bottom plates (available from Hampton Research (Aliso Viejo, Calif.)), the 24 well VDX plates (available from Hampton Research (Aliso Viejo, Calif.)), and and NeXtal EasyXtal 15 well plates (available from Molecular Dimensions (Maumee, Ohio)). The volume of the reservoir for the 384-well plate can be 50 μL. The volume of the reservoir for the 96-well low profile plate can be 100 μL, and for the CrystalQuick™ plates it can be varied between 70-100 μL. Crystals can also be grown in 72-well terasaki plates using the microbatch method. They also can be grown in 96-well Corning® (available from Hampton Research (Aliso Viejo, Calif)) with a reservoir of 50 μL.

According to one aspect, the invention provides for a crystal with unit cell dimensions unit cell dimensions of a=38.155±2Å; b=72.394±2Å; c=103.946±2Å; a=90°; b=90°; g=90° and space group space group p 2 21 21. Preferably, the crystal comprises the BTK-Compound I complex.

It will be readily apparent to those skilled in the art that the unit cells of the crystal compositions may deviate up to ±1-2 Å in cell length from the above cell dimensions depending on the deviation in the unit cell calculations or conformational change in the protein.

The BTK protein may be produced by any well-known method, including synthetic methods, such as solid phase, liquid phase and combination solid phase/liquid phase syntheses; recombinant DNA methods, including cDNA cloning, optionally combined with site directed mutagenesis; and/or purification of the natural products. In one embodiment, the protein is overexpressed in a baculovirus system.

### Methods of Obtaining Crystals of BTK or Complexes Thereof

The invention also relates to a method of obtaining a crystal of a BTK protein comprising the steps of:
a) producing and purifying a BTK protein;
b) combining a crystallizable solution with said BTK protein to produce a crystallizable composition; and
c) subjecting said crystallizable composition to conditions which promote crystallization and obtaining said crystals.

The invention also relates to a method of obtaining a crystal of a BTK protein complex, further comprising the step of: d) soaking said crystal in a buffer solution comprising the compound of Formula (I) I.

In some embodiments, a method of obtaining a crystal of a BTK protein complex comprises mixing a crystal of the compound of Formula (I) and a conformer. In some embodiments, the cofomer comprises saccharine, maleic acid, glycine, sulfacetamide, serine, ketoglutaric acid, orotic acid, maltol, urea, proline, nicotinic acid, L-lysine, isonicotinic acid, benzoic acid, nicotinamide, salicylic acid, isonicotinamide, 3-hydroxybenzoic acid, L-tartaric acid, 4-aminobenzoic acid, L-malic acid, succinic acid, citric acid, 2,5-dihydroxybenzoic acid, L-lactic acid, 2,4-dihydroxybenzoic acid, caffeine, sorbic acid, or L-glutamic acid. In some embodiments the mixture of a crystal of the compound of Formula (I) and a conformer are wetted using ethanol. In some embodiments, the wetted mixture of a crystal of the compound of Formula (I) and a conformer is subjected to grinding to form a ground mixture of a crystal of the compound of Formula (I) and a conformer. In some embodiments, the wetted mixture of a crystal of the compound of Formula (I) and a conformer is subjected to heating and cooling. In some embodiments, the ground mixture of a crystal of the compound of Formula (I) and a conformer is air dried to form a dried mixture of a crystal of the compound of Formula (I) and a conformer. In some embodiments, the air dried mixture of a crystal of the compound of Formula (I) and a conformer is analyzed by XPRD. Some embodiments further comprise filtering the mixture of a crystal of the compound of Formula (I) and a conformer prior to drying

The invention also relates to a method of obtaining a crystal of a BTK protein complex, comprising the steps of:
a) producing and purifying a BTK protein;
b) combining a crystallizable solution with said BTK protein thereof in the presence of the compound of Formula (I) to produce a crystallizable composition; and
c) subjecting said crystallizable composition to conditions which promote crystallization and obtaining said crystals.

In certain embodiments, the method of making crystals of a BTK protein, or complexes thereof, includes the use of a device for promoting crystallizations. Devices for promoting crystallization can include but are not limited to the hanging-drop, sitting-drop, dialysis or microtube batch devices. (U.S. Pat. Nos. 4,886,646, 5,096,676, 5,130,105, 5,221,410 and 5,400,741; Pay et al., Proteins: Structure, Function, and Genetics 20: 98-102 (1994), incorporated herein by reference). The hanging-drop, sitting-drop, and some adaptations of the microbatch methods (D'Arcy et al., J. Cryst. Growth 168: 175-180 (1996) and Chayen, J. Appl. Cryst. 30: 198-202 (1997)) produce crystals by vapor diffusion. The hanging drop and sitting drop containing the crystallizable composition is equilibrated in a reservoir containing a higher or lower concentration of the precipitant. As the drop approaches equilibrium with the reservoir, the saturation of protein in the solution leads to the formation of crystals.

Microseeding or seeding may be used to increase the size and quality of crystals. In this instance, micro-crystals are crushed to yield a stock seed solution. The stock seed solution is diluted in series. Using a needle, glass rod, micro-pipet, micro-loop or strand of hair, a small sample from each diluted solution is added to a set of equilibrated drops containing a protein concentration equal to or less than a concentration needed to create crystals without the presence of seeds. The aim is to end up with a single seed crystal that will act to nucleate crystal growth in the drop.

It would be readily apparent to one of skill in the art to vary the crystallization conditions disclosed above to identify other crystallization conditions that would produce crystals of a BTK protein or BTK protein complex. Such variations include, but are not limited to, adjusting pH, protein concentration and/or crystallization temperature, changing the identity or concentration of salt and/or precipitant used, using a different method of crystallization, or introducing additives such as detergents (e.g., TWEEN 20 (monolaurate), LDAO, Brij 30 (4 lauryl ether)), sugars (e.g., glucose, maltose), organic compounds (e.g., dioxane, dimethylformamide), lanthanide ions or polyionic compounds that aid in crystallization. High throughput crystallization assays may also be used to assist in finding or optimizing the crystallization condition.

### Binding Pockets of BTK

As disclosed herein, applicants have provided the three-dimensional X-ray structure of BTK-Compound I complex. The atomic coordinates for the structures of the BTK Compound I complex are presented in Table 2.

To use the structure coordinates generated for the BTK-Compound I complex or one of its binding pockets, it may be necessary to convert the structure coordinates, or portions thereof, into a three-dimensional shape (i.e., a three-dimensional representation of these complexes or binding pockets). This is achieved through the use of a computer and commercially available software that is capable of generating the three-dimensional representations or structures of molecules or molecular complexes, or portions thereof, from a set of structural coordinates. These three-dimensional representations may be displayed on a computer screen.

Binding pockets, also referred to as binding sites in the present invention, are of significant utility in fields such as drug discovery. The association of natural ligands or substrates with the binding pockets of their corresponding receptors or enzymes is the basis of many biological mechanisms of action. Similarly, many drugs exert their biological effects through association with the binding pockets of receptors and enzymes. Such associations may occur with all or part of the binding pocket. An understanding of such associations will help lead to the design of drugs having more favorable associations with their target receptor or enzyme, and thus, improved biological effects. Therefore, this information is valuable in designing potential inhibitors of the binding pockets of biologically important targets. The binding pockets of this invention will be important for drug design.

The conformations of the BTK protein and other proteins at a particular amino acid site, along the polypeptide backbone, can be compared using well-known procedures for performing sequence alignments of the amino acids. Such sequence alignments allow for the equivalent sites on these proteins to be compared. Such methods for performing sequence alignment include, but are not limited to, the “bestfit” program and CLUSTAL W Alignment Tool, Higgins et al., supra.

In one embodiment, the BTK binding pocket is made up of the BTK kinase domain and comprises amino acid residues Leu408, Gly409, Thr410, Gly411, Va1416, Ala428, Lys430, Asn439, Met449, Leu452, Va1458, Ile472, Thr474, Glu475, Tyr476, Met477, Gly480, Cys481, Asn 484, Arg525, Leu528, Ser538, Asp539, and Phe540.according to the structure of the BTK-Compound I complex in Table 2.

It will be readily apparent to those of skill in the art that the numbering of amino acid residues in homologues of human BTK may be different than that set forth for human BTK. Corresponding amino acids in BTK homologues are easily identified by visual inspection of the amino acid sequences or by using commercially available homology software programs. Homologues of BTK include, for example, BTK from other species, such as non-humans primates, mouse, rat, etc.

Those of skill in the art understand that a set of structure coordinates for an enzyme or an enzyme-complex, or a portion thereof, is a relative set of points that define a shape in three dimensions. Thus, it is possible that an entirely different set of coordinates could define a similar or identical shape. Moreover, slight variations in the individual coordinates will have little effect on overall shape. In terms of binding pockets, these variations would not be expected to significantly alter the nature of ligands that could associate with those pockets.

The variations in coordinates discussed above may be generated because of mathematical manipulations of the BTK-Compound I complex structure coordinates. For example, the structure coordinates set forth in Table 2 may undergo crystallographic permutations of the structure coordinates, fractionalization of the structure coordinates, integer additions or subtractions to sets of the structure coordinates, inversion of the structure coordinates or any combination of the above.

Alternatively, modifications in the crystal structure due to mutations, additions, substitutions, and/or deletions of amino acids, or other changes in any of the components that make up the crystal may also account for variations in structure coordinates. If such variations are within a certain root mean square deviation as compared to the original coordinates, the resulting three-dimensional shape is considered encompassed by this invention. Thus, for example, a ligand that bound to the kinase domain of BTK would also be expected to bind to another binding pocket whose structure coordinates defined a shape that fell within the RMSD value.

Various computational analyses may be necessary to determine whether a molecule or binding pocket, or portion thereof, is sufficiently similar to the binding pockets above-described. Such analyses may be carried out in well-known software applications, such as ProFit (ProFit version 1.8, available from A.C.R. Martin, University College London); Swiss-Pdb Viewer (Guex and Peitsch, Electrophoresis 18: 2714-2723 (1997)); the Molecular Similarity application of QUANTA (Accelrys, San Diego, Calif. ©2001, 2002) and as described in the accompanying User's Guide, which are incorporated herein by reference.

The above programs permit comparisons between different structures, different conformations of the same structure, and different parts of the same structure. The procedure used in QUANTA (Accelrys, San Diego, Calif. ©2001, 2002) and Swiss-Pdb Viewer (Guex and Peitsch, Electrophoresis 18: 2714-2723 (1997) to compare structures is divided into four steps: 1) load the structures to be compared; 2) define the atom equivalences in these structures; 3) perform a fitting operation on the structures; and 4) analyze the results.

The procedure used in ProFit to compare structures includes the following steps: 1) load the structures to be compared; 2) specify selected residues of interest; 3) define the atom equivalences in the selected residues; 4) perform a fitting operation on the selected residues; and 5) analyze the results.

Each structure in the comparison is identified by a name. One structure is identified as the target (i.e., the fixed structure); all remaining structures are working structures (i.e., moving structures). Since atom equivalency within QUANTA (Accelrys, San Diego, Calif. ©2001, 2002) is defined by user input, for the purposes of this invention, we will define equivalent atoms as protein backbone atoms N, O, C and Cα for all corresponding amino acid residues between two structures being compared.

The corresponding amino acids may be identified by sequence alignment programs such as the “bestfit” program available from the Genetics Computer Group which uses the local homology algorithm described by Smith and Waterman in Advances in Applied Mathematics 2: 482 (1981), which is incorporated herein by reference. A suitable amino acid sequence alignment will require that the proteins being aligned share minimum percentage of identical amino acids. Generally, a first protein being aligned with a second protein should share in excess of about 35% identical amino acids (Hanks et al., Science 241: 42 (1988); Hanks and Quinn, Methods in Enzymology 200: 38 (1991)). The identification of equivalent residues can also be assisted by secondary structure alignment, for example, aligning the α-helices, β-sheets in the structure. The program Swiss-Pdb viewer (Guex and Peitsch, Electrophoresis 18: 2714-2723 (1997) utilizes a best fit algorithm that is based on secondary sequence alignment.

When a rigid fitting method is used, the working structure is translated and rotated to obtain an optimum fit with the target structure. The fitting operation uses an algorithm that computes the optimum translation and rotation to be applied to the moving structure, such that the root mean square difference of the fit over the specified pairs of equivalent atom is an absolute minimum. This number, given in angstroms, is reported by the above programs. The Swiss-Pdb Viewer program (Guex and Peitsch, Electrophoresis 18: 2714-2723 (1997) sets an RMSD cutoff for eliminating pairs of equivalent atoms that have high RMSD values. An RMSD cutoff value can be used to exclude pairs of equivalent atoms with extreme individual RMSD values. In the program ProFit, the RMSD cutoff value can be specified by the user.

For the purpose of this invention, any molecule, molecular complex, binding pocket, motif, domain thereof or portion thereof that is within a root mean square deviation for backbone atoms (N, Cα, C, O) when superimposed on the relevant backbone atoms described by structure coordinates listed in Table 2 are encompassed by this invention.

One aspect of this invention provides a crystalline molecule comprising a protein defined by structure coordinates of a set of amino acid residues that are identical to BTK amino acid residues according to Table 2, wherein the RMSD between backbone atoms of said set of amino acid residues and said BTK amino acid residues is not more than about 3.0 Å. In other embodiments, the RMSD between backbone atoms of said set of amino acid residues and said BTK amino acid residues is not greater than about 2.0 Å, not greater than about 1.5 Å, not greater than about 1.1 Å, not greater than about 1.0 Å, not greater than about 0.9 Å, not greater than about 0.8 Å, not greater than about 0.7 Å, not greater than about 0.6 Å, or not greater than about 0.5 Å. Calculations of RMSD values were done with Swiss Pdb Viewer (Guex and Peitsch, Electrophoresis 18: 2714-2723 (1997)).

In one embodiment, the present invention provides a crystalline molecule comprising all or part of a binding pocket defined by a set of amino acid residues comprising amino acid residues which are identical to human BTK amino acid residues Leu408, Gly409, Thr410, Gly411, Va1416, Ala428, Lys430, Asn439, Met449, Leu452, Va1458, Ile472, Thr474, Glu475, Tyr476, Met477, Gly480, Cys481, Asn 484, Arg525, Leu528, Ser538, Asp539, and Phe540 according to Table 2, wherein the RMSD of the backbone atoms between said BTK amino acid residues and said amino acid residues which are identical is not greater than about 2.5 Å. In other embodiments, the RMSD is not greater than about 2.4 Å, 2.2 Å, 2.0 Å, 1.8 Å, 1.6 Å, 1.4 Å, 1.2 Å, 1.0 Å, 0.8 Å, 0.5 Å, 0.3 Å, or 0.2 Å. In other embodiments, the binding pocket is defined by a set of amino acid residues comprising at least four, six, eight, ten, twelve, fifteen, twenty, twenty-five, thirty, thirty-five, forty, forty-five or fifty amino acid residues which are identical to said BTK amino acid residues.

### Method of Making BTK Crystalline Compositions

The BTK protein may be produced by any well-known method, including synthetic methods, such as solid phase, liquid phase and combination solid phase/liquid phase syntheses; recombinant DNA methods, including cDNA cloning, optionally combined with site directed mutagenesis; and/or purification of the natural products. In an aspect, the protein is overexpressed in a baculovirus system or an E. coli system. In another aspect, the protein is overexpressed in a baculovirus system.

The invention also provides a method of making crystals of BTK protein in the presence or absence of a chemical entity (e.g. Compoundl). Such methods comprise the steps of: a) producing and purifying BTK protein; b) combining said BTK protein, or a homolog thereof in the presence or absence of a chemical entity with a crystallization solution to produce a crystallizable composition; and c) subjecting said crystallizable composition to, conditions which promote crystallization.

The crystallization solution may include, but is not limited to, polyethylene glycol (PEG) at between about 10% to 35% v/v, 100-300 mM ammonium sulphate and a buffer that maintains pH at between about 4.0 and 8.0. In one embodiment, the crystallization solution comprises 31% PEG MME 5K, 100 mM 2-(N-morpholino) ethanesulfonic acid (MES) at pH 6.75 and 200 mM ammonium sulphate.

According to one embodiment, the crystallizable composition comprises BTK protein in the presence or absence of a chemical entity (e.g. Compound I). In another embodiment, the crystallizable composition comprises BTK protein and a chemical entity. In one embodiment, the crystallizable composition further comprises a precipitant, polyethylene glycol (PEG) at between about 10 to 30% v/v, 100-300 mM ammonium sulphate and a buffer that maintains pH at between about 4.0 and 8.0, and optionally a reducing agent, such as dithiothreitol (DTT) at between about 1 to 20 mM. The BTK protein may be further modified to include posttranslation modificiations. The BTK protein or complex is preferably 85-100% pure prior to forming the composition. More preferably, the BTK protein or complex is 90-100% pure. Even more preferably, the BTK protein or complex is 95-100% pure.

It would be readily apparent to one of skill in the art to vary the crystallization conditions disclosed above to identify other crystallization conditions that would produce crystals of BTK protein or a homolog thereof in the presence or absence of a ligand. Such variations include, but are not limited to, adjusting pH, protein concentration and/or crystallization temperature, changing the identity or concentration of salt and/or precipitant used, using a different method for crystallization, or introducing additives such as detergents (e.g., TWEEN®20 (monolaurate), LDOA, BRIJ®30 (4 lauryl ether)), sugars (e.g., glucose, maltose), organic compounds (e.g., dioxane, dimethylformamide), lanthanide ions, or poly-ionic compounds that aid in crystallizations. High throughput crystallization assays may also be used to assist in finding or optimizing the crystallization condition.

Methods of Identifying and/or designing of Candidate Inhibitors of BTK

A further aspect of the present invention is a method for identifying and/or designing a candidate inhibitor using a human BTK crystal comprising a human BTK protein, wherein said method comprises: a) preparing the crystalline composition of BTK and Compound I and b) soaking another candidate inhibitor into the crystalline composition, displacing the compound of Formula (I) (original ligand) to form an inhibitor-crystal complex, c) determining the three-dimensional structure coordinates of the inhibitor-crystal complex prepared in step b); and d) using the structure coordinates from step c) to design and/or identifying a candidate inhibitor; and e) contacting said candidate inhibitor with human BTK and measuring the ability of said candidate inhibitor to bind to BTK.

In one aspect, a molecular complex comprising: BTK with a ligand. In one aspect, a molecular complex comprising SEQ ID NO: 3, and a ligand, wherein said molecular complex forms a crystalline composition characterized with space group p 2 21 21 and has unit cell parameters a=38.155±2Å; b=72.394±2Å; c=103.946±2Å; a=90°; b=90°; g=90°, wherein said crystalline solution is buffered at between pH 5.0-7.0 and comprises 10-15% PEG and 50 mM ammonium sulphate. In one aspect, a molecular complex comprises SEQ ID NO: 3, and a ligand, wherein the ligand is a compound of Formula (I):

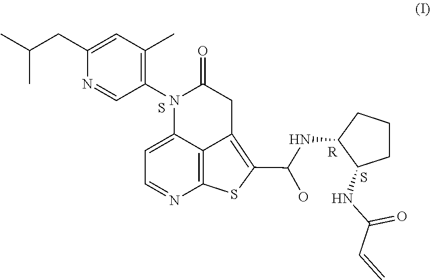

or pharmaceutically acceptable salts, hydrates, polymorphs or solvates thereof

In some aspects, the molecular complex of BTK with Compound I bound to a binding site provides important structural information for the development of novel BTK inhibitors. The invention comprises a molecular complex comprising SEQ ID NO:3, and Compound I, wherein said molecular complex is characterized with space group p 2 21 21 and has unit cell parameters a=38.155±2Å; b=72.394±2Å; c=103.946±2Å; a=90°; b=90°; g=90°.

Persons skilled in the art will recognize that modification at C terminus and N terminus is possible, as well as some amino acid residue mutations, substitutions, including glycosylations, acylations, and methylations. All such modifications are considered to be equivalents within the scope of the invention.

One aspect is a method for identifying a candidate inhibitor of BTK, wherein said method comprises: generating a three-dimensional structure of a binding site of BTK on a computer, wherein the three dimensional structure coordinates of possess the unit cell and space group parameters of the crystalline composition comprising SEQ ID NO:3, and a ligand, wherein the ligand is a compound of Formula (I):

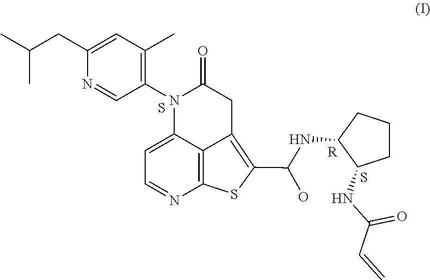

or pharmaceutically acceptable salts, hydrates, polymorphs or solvates thereof, and wherein said crystalline composition is characterized with space group p 2 21 21 and has unit cell parameters a=38.155±2Å; b=72.394±2Å; c=103.946±2Å; a=90°; b=90°; g=90°; employing said three dimensional structure to design or select a candidate inhibitor; and contacting said candidate inhibitor with human BTK and measuring the ability of said candidate inhibitor to bind to BTK.

In another aspect, a method for identifying a candidate inhibitor of BTK, wherein said method comprises: generating a three-dimensional structure of a binding site of BTK on a computer, wherein the three dimensional structure coordinates of possess the unit cell and space group parameters of the crystalline composition comprising SEQ ID NO:3, and a ligand, wherein said crystalline composition is characterized with space group p 2 21 21 and has unit cell parameters a=38.155±2Å; b=72.394±2Å; c=103.946±2Å; a=90°; b=90°; g=90°; employing said three dimensional structure to design or select a candidate inhibitor, wherein the candidate inhibitor makes a direct covalent bond with Cys481, wherein the candidate inhibitor makes a hydrogen bond with Lys430, Met477, Asp539, or any combination thereof; and contacting said candidate inhibitor with human BTK and measuring the ability of said candidate inhibitor to bind to BTK.

### Methods of Computational Identification of Candidate Inhibitors of BTK

According to another aspect, this invention provides a machine-readable data storage medium, comprising a data storage material encoded with machine-readable data, wherein said data defines the above-mentioned molecules or molecular complexes. In one embodiment, the data defines the above-mentioned binding pockets by comprising the structure coordinates of said amino acid residues according to Table 2. To use the structure coordinates generated for BTK, or BTK kinase domain, it is at times necessary to convert them into a three-dimensional shape or to extract three-dimensional structural information from them. This is achieved through the use of commercially or publicly available software that is capable of generating a three-dimensional structure or a three-dimensional representation of molecules or portions thereof from a set of structure coordinates. In one embodiment, three-dimensional structure or representation may be displayed graphically.

Therefore, according to another embodiment, this invention provides a machine-readable data storage medium comprising a data storage material encoded with machine readable data. In one embodiment, a machine programmed with instructions for using said data is capable of generating a three-dimensional structure or three-dimensional representation of any of the molecules, or molecular complexes or binding pockets thereof, that are described herein.

This invention also provides a computer comprising:
(a) a machine-readable data storage medium, comprising a data storage material encoded with machine-readable data, wherein said data defines any one of the above molecules or molecular complexes;
(b) a working memory for storing instructions for processing said machine-readable data;
(c) a central processing unit (CPU) coupled to said working memory and to said machine-readable data storage medium for processing said machine readable data and means for generating three-dimensional structural information of said molecule or molecular complex; and
(d) output hardware coupled to said central processing unit for outputting three-dimensional structural information of said molecule or molecular complex, or information produced by using said three-dimensional structural information of said molecule or molecular complex.

In one aspect, the data defines the binding pocket of the molecule or molecular complex.

Three-dimensional data generation may be provided by an instruction or set of instructions such as a computer program or commands for generating a three-dimensional structure or graphical representation from structure coordinates, or by subtracting distances between atoms, calculating chemical energies for a BTK molecule or molecular complex, or calculating or minimizing energies for an association of a BTK molecule or molecular complex thereof to a chemical entity such a Compound I. The graphical representation can be generated or displayed by commercially available software programs. Examples of software programs include but are not limited to QUANTA (Accelrys, San Diego, Calif. ©2001, 2002), O(Jones et al., Acta Crystallogr. A47: 110-119 (1991)) and RIBBONS (Carson, J. Appl. Crystallogr. 24: 958-961 (1991)), which are incorporated herein by reference. Certain software programs may imbue this representation with physico-chemical attributes which are known from the chemical composition of the molecule, such as residue charge, hydrophobicity, torsional and rotational degrees of freedom for the residue or segment, etc. Examples of software programs for calculating chemical energies are described in the Rational Drug Design section.

Information of said binding pocket or information produced by using said binding pocket can be outputted through display terminals, touchscreens, facsimile machines, modems, CD-ROMs, printers, a CD or DVD recorder, ZIP™ or JAZ™ drives or disk drives. The information can be in graphical or alphanumeric form.

In one embodiment, the computer is executing an instruction such as a computer program for generating three-dimensional structure or docking. In another embodiment, the computer further comprises a commercially available software program to display the information as a graphical representation. Examples of software programs include but as not limited to, QUANTA (Accelrys, San Diego, Calif. ©2001, 2002), O (Jones et al., Acta Crystallogr. A47: 110-119 (1991)) and RIBBONS (Carson, J. Appl. Crystallogr. 24: 958-961 (1991)), all of which are incorporated herein by reference.

Thus, in accordance with the present invention, data capable of generating the three-dimensional structure or three-dimensional representation of the above molecules or molecular complexes, or binding pockets thereof, can be stored in a machine-readable storage medium, which is capable of displaying structural information or a graphical three-dimensional representation of the structure. In one embodiment, the means of generating three-dimensional information is provided by the means for generating a three-dimensional structural representation of the binding pocket or protein of a molecule or molecular complex.

Another aspect of the present invention is a method for identifying a candidate inhibitor of BTK, wherein said method comprises: generating a three-dimensional structure of a binding site of BTK on a computer, wherein the three dimensional structure coordinates of possess the unit cell and space group parameters of the crystalline composition of the present invention, employing said three dimensional structure to design or select a candidate inhibitor; and contacting said candidate inhibitor with human BTK and measuring the ability of said candidate inhibitor to bind to BTK.

The invention, as disclosed herein, will be useful for identifying BTK inhibitors and to study the role of BTK in cell signaling. In order to use the structure coordinates generated for BTK, or a BTK complex, it is often times necessary to convert the structure coordinates into a three-dimensional shape. This is achieved through the use of commercially available software that is capable of generating three-dimensional graphical representations of molecules or portions thereof from a set of structure coordinates.

Binding pockets, also referred to as binding sites in the present invention, are of significant utility in fields such as drug discovery. The association of natural ligands or substrates with the binding pockets of their corresponding receptors or enzymes is the basis of many biological mechanisms of action. Similarly, many drugs exert their biological effects through association with the binding pockets of receptors and enzymes. Such associations may occur with all or part of the binding pocket. An understanding of such associations will help lead to the design of drugs having more favorable associations with their target receptor or enzyme, and thus, improved biological effects. Therefore, this information is valuable in designing potential inhibitors of the binding pockets of biologically important targets.

In one aspect, part of the binding pocket is at least two amino acid residues, preferably, amino acid residues 389-659of BTK which correspond to the kinase domain of BTK. In another aspect, the binding pocket is represented by SEQ ID NO.3.

Another aspect is a method for identifying a candidate inhibitor of BTK, wherein said method comprises: generating a three-dimensional structure of a binding site of BTK on a computer, wherein the three dimensional structure coordinates of possess the unit cell and space group parameters of the crystalline composition comprising SEQ ID NO:3, and a ligand such as Compound I, wherein said crystalline composition is characterized with space group p 2 21 21 and has unit cell parameters a=38.155±2Å; b=72.394±2Å; c=103.946±2Å; a=90°; b=90°; g=90°, employing said three dimensional structure to design or select a candidate inhibitor, wherein the candidate inhibitor makes a direct covalent bond with Cys481, and wherein the candidate inhibitor makes a hydrogen bond with Lys430, and wherein the candidate inhibitor makes a hydrogen bond with Met477, and wherein the candidate inhibitor makes a hydrogen bond with Asp539; and contacting said candidate inhibitor with human BTK and measuring the ability of said candidate inhibitor to bind to BTK.

Another aspect is a method for identifying a candidate inhibitor of BTK, wherein said method comprises: generating a three-dimensional structure of a binding site of BTK on a computer, wherein the three dimensional structure coordinates of possess the unit cell and space group parameters of the crystalline composition comprising SEQ ID NO:3, and a ligand such as Compound I, wherein said crystalline composition is characterized with space group p 2 21 21 and has unit cell parameters a=38.155±2Å; b=72.394±2Å; c=103.946±2Å; a=90°; b=90°; g=90°; employing said three dimensional structure to design or select a candidate inhibitor, wherein the candidate inhibitor makes a direct covalent bond with Cys481, and wherein the candidate inhibitor makes a hydrogen bond with Lys430, and wherein the candidate inhibitor makes a hydrogen bond with Met477, and wherein the candidate inhibitor makes a hydrogen bond with Asp539; and contacting said candidate inhibitor with human BTK and measuring the ability of said candidate inhibitor to bind to BTK.

Another aspect is a method for identifying and/or designing a candidate inhibitor using a human BTK crystal comprising a human BTK protein, wherein said method comprises: a) preparing a crystalline composition comprising SEQ ID NO:3, and a ligand such as Compound I, wherein said crystalline composition is characterized with space group p 2 21 21 and has unit cell parameters a=38.155±2Å; b=72.394±2Å; c=103.946±2Å; a=90°; b=90°; g=90°, wherein the ligand is a compound of Formula (I):

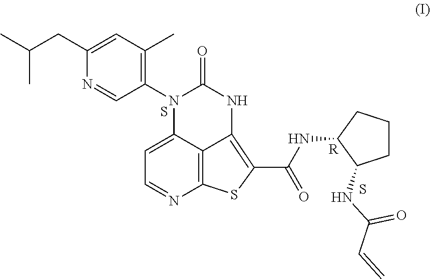

or pharmaceutically acceptable salts, hydrates, polymorphs or solvates thereof; b) soaking another candidate inhibitor into the crystalline composition, displacing the compound of Formula (I) (original ligand) to form an inhibitor-crystal complex; c) determining the three-dimensional structure coordinates of the inhibitor-crystal complex prepared in step b); d) using the structure coordinates from step b) to design or identify a candidate inhibitor; and e) contacting said candidate inhibitor with human BTK and measuring the ability of said candidate inhibitor to bind to BTK.

The BTK structure coordinates or the three-dimensional graphical representation generated from these coordinates may be used in conjunction with a computer for a variety of purposes, including drug discovery.

For example, the structure encoded by the data may be computationally evaluated for its ability to associate with chemical entities. Chemical entities that associate with BTK may inhibit BTK or its homologs, and are potential drug candidates. Alternatively, the structure encoded by the data may be displayed in a graphical three-dimensional representation on a computer screen. This allows visual inspection of the structure, as well as visual inspection of the structure's association with chemical entities.

According to another aspect, the invention provides a method for designing, selecting and/or optimizing a chemical entity that binds to all or part of the molecule or molecular complex comprising the steps of: (a) providing the structure coordinates of said molecule or molecular complex on a computer comprising the means for generating three-dimensional structural information of all or part of said molecule or molecular complex from said structure coordinates; (b) designing, selecting and/or optimizing said chemical entity by employing means for performing a fitting operation between said chemical entity and said three-dimensional structural information of all or part of said molecule or molecular complex; and (c) contacting said candidate inhibitor with human BTK and measuring the ability of said candidate inhibitor to bind to BTK.

In another aspect, the method comprises the steps of: (a) constructing a computer model of the binding pocket of BTK; (b) selecting a chemical entity to be evaluated by de novo ligand design, or by modifying a known agonist or inhibitor, or a portion thereof; (c) employing computational means to perform a fitting operation between computer models of said chemical entity to be evaluated and said binding pocket in order to provide an energy-minimized configuration of said chemical entity in the binding pocket; and (d) contacting said candidate inhibitor with human BTK and measuring the ability of said candidate inhibitor to bind to BTK.

Three-dimensional structural information in step (a) may be generated by instructions such as a computer program or commands that can generate a three-dimensional structure or graphical representation; subtract distances between atoms; calculate chemical energies for the BTK molecule, molecular complex or homologs thereof; or calculate or minimize energies of an association of the BTK molecule, molecular complex or homologs thereof to a chemical entity. These types of computer programs are known in the art. The graphical representation can be generated or displayed by commercially available software programs. Examples of software programs include but are not limited to 1. Coot, (Paul Emsley and Bernhard Lohkamp and William G. Scott and Kevin Cowtan, Features and Development of Coot, Acta Crystallographica Section D—Biological Crystallography, 2010, vol 66, pp 486-501), 2. PyMOL (The PyMOL Molecular Graphics System, Schrodinger, LLC), and 3. MOE (Molecular Operating Environment) Molecular Operating Environment (MOE), 2019.01; Chemical Computing Group ULC. Certain software programs may imbue this representation with physico-chemical attributes which are known from the chemical composition of the molecule, such as residue charge, hydrophobicity, torsional and rotational degrees of freedom for the residue or segment, etc.

The design of compounds that bind to or inhibit BTK binding pockets according to this invention generally involves consideration of two factors. First, the chemical entity must be capable of physically and structurally associating with parts or all of the BTK binding pocket. Non-covalent molecular interactions important in this association include hydrogen bonding, van der Waals interactions, hydrophobic interactions and electrostatic interactions.

Second, the chemical entity must be able to assume a conformation that allows it to associate with the BTK binding pocket directly. Although certain portions of the chemical entity will participate indirectly through one or more water molecules, those portions of the chemical entity may still influence the overall binding of the chemical entity to BTK. This, in turn, may have a significant impact on potency. Such conformational requirements include the overall three-dimensional structure and orientation of the chemical entity in relation to all or a portion of the binding pocket.

Using a multiple alignment program to compare each BTK structure and structures of other members of the protein family. To compare structures, first, a sequence alignment between protein sequences is performed. Second, a putative core is constructed by superimposing a series of corresponding structures in the protein family. Third, residues of high spatial variation are discarded, and the core alignment is iteratively refined. The amino acids that make up the final core structure have low structural variance and have the same local and global conformation relative to the corresponding residues in the protein family.

In one aspect, the binding pocket comprises SEQ ID NO.3. In another aspect the binding pocket comprises amino acid residues 389-659 of BTK representing the kinase domain of BTK.

Those of skill in the art understand that a set of structure coordinates for a molecule or a molecular-complex or a portion thereof, is a relative set of points that define a shape in three dimensions. Thus, it is possible that an entirely different set of coordinates could define a similar or identical shape. Moreover, slight variations in the individual coordinates will have little effect on overall shape. In terms of binding pockets, these variations would not be expected to significantly alter the nature of ligands that could associate with those pockets.

The variations in coordinates discussed above may be generated as a result of mathematical manipulations of the BTK structure coordinates. For example, the structure coordinates could be manipulated by crystallographic permutations of the structure coordinates, fractionalization of the structure coordinates, integer additions or subtractions to sets of the structure coordinates, inversion of the structure coordinates or any combination of the above.

Alternatively, modifications in the crystal structure due to mutations, additions, substitutions, and/or deletions of amino acids, or other changes in any of the components that make up the crystal could also account for variations in structure coordinates. If such variations are within a certain root mean square deviation as compared to the original coordinates, the resulting three-dimensional shape is considered encompassed by this invention. Thus, for example, a ligand that binds to the binding pocket of BTK would also be expected to bind to another binding pocket whose structure coordinates define a shape that falls within the acceptable root mean square deviation.

Various computational analyses may be necessary to determine whether a binding pocket, motif, domain or portion thereof of a molecule or molecular complex is sufficiently similar to the binding pocket, motif, domain or portion thereof of BTK such as the kinase domain of BTK. Such analyses may be carried out using well known software applications, such as ProFit (A. C. R. Martin, SciTech Software, ProFit version 1.8, University College London, www.bioinf.org.uk/software), Swiss-Pdb Viewer (Guex et al., Electrophoresis, 18, pp. 2714-2723 (1997)), the Molecular Similarity application of QUANTA (Molecular Simulations Inc., San Diego, Calif. ©1998, 2000) and as described in the accompanying User's Guide, which are incorporated herein by reference.

The above programs permit comparisons between different structures, different conformations of the same structure, and different parts of the same structure. The procedure used in QUANTA (Molecular Simulations, Inc., San Diego, Calif. 01998, 2000) and Swiss-Pdb Viewer to compare structures is divided into four steps: 1) load the structures to be compared; 2) define the atom equivalences in these structures; 3) per form a fitting operation on the structures; and 4) analyze the results.

The procedure used in ProFit to compare structures includes the following steps: 1) load the structures to be compared; 2) specify selected residues of interest; 3) define the atom equivalences in the selected residues; 4) perform a fitting operation on the selected residues; and 5) analyze the results.

Each structure in the comparison is identified by a name. One structure is identified as the target (i.e., the fixed structure); all remaining structures are working structures (i.e., moving structures). Since atom equivalency within the above programs is defined by user input, for the purpose of this invention we will define equivalent atoms as protein back bone atoms (N, Ca, C and O) for BTK amino acids and corresponding amino acids in the structures being compared.

The corresponding amino acids may be identified by sequence alignment programs such as the “bestfit” program available from the Genetics Computer Group which uses the local homology algorithm described by Smith and Waterman in Advances in Applied Mathematics 2, 482 (1981), which is incorporated herein by reference. A suitable amino acid sequence alignment will require that the proteins being aligned share minimum percentage of identical amino acids. Generally, a first protein being aligned with a second protein should share in excess of about 35% identical amino acids (Hanks et al., Science, 241, 42 (1988); Hanks and Quinn, Methods in Enzymology, 200, 38 (1991)). The identification of equivalent residues can also be assisted by secondary structure alignment, for example, aligning the a-helices, (3-sheets in the structure. The program Swiss-Pdb Viewer has its own best fit algorithm that is based on secondary sequence alignment.

When a rigid fitting method is used, the working structure is translated and rotated to obtain an optimum fit with the target structure. The fitting operation uses an algorithm that computes the optimum translation and rotation to be applied to the moving structure, such that the root mean square difference of the fit over the specified pairs of equivalent atom is an absolute minimum. This number, given in angstroms, is reported by the above programs. The Swiss-Pdb Viewer program sets an RMSD cutoff for eliminating pairs of equivalent atoms that have high RMSD values. An RMSD cutoff value can be used to exclude pairs of equivalent atoms with extreme individual RMSD values. In the program ProFit, the RMSD cutoff value can be specified by the user.

For the purpose of this invention, any molecule, molecular complex, binding pocket, motif, domain thereof or portion thereof that is within a root mean square deviation for back bone atoms (N, Cα, C, O) when superimposed on the relevant backbone atoms described by structure coordinates encompassed by this invention.

Therefore, one aspect of this invention provides a molecule or molecular complex comprising all or part of a BTK binding pocket defined by structure coordinates of a set of amino acid residues that correspond to BTK amino acid residues 389-659, wherein the root mean square deviation of the backbone atoms between said amino acids of said molecule or molecular complex and said BTK amino acids is not more than about 3.0 Å. In one embodiment, the RMSD is not greater than about 2.0 Å. In one embodiment, the RMSD is not greater than about 1.0 Å. In one embodiment, the RMSD is not greater than about 0.8 Å. In one embodiment, the RMSD is not greater than about 0.5 Å. In one embodiment, the RMSD is not greater than about 0.3 Å. In one embodiment, the RMSD is not greater than about 0.2 Å.

Therefore, one aspect of this invention provides a molecule or molecular complex comprising all or part of a BTK binding pocket defined by structure coordinates of a set of amino acid residues that correspond to SEQ ID NO.3, wherein the root mean square deviation of the backbone atoms between said amino acids of said molecule or molecular complex and said BTK amino acids is not more than about 3.0 Å. In one embodiment, the RMSD is not greater than about 2.0 Å. In one embodiment, the RMSD is not greater than about 1.0 Å. In one embodiment, the RMSD is not greater than about 0.8 Å. In one embodiment, the RMSD is not greater than about 0.5 Å. In one embodiment, the RMSD is not greater than about 0.3 Å. In one embodiment, the RMSD is not greater than about 0.2 Å.

### Rational Drug Design

The BTK structure coordinates or the three-dimensional graphical representation generated from these coordinates may be used in conjunction with a computer for a variety of purposes, including drug discovery.

For example, the structure encoded by the data may be computationally evaluated for its ability to associate with chemical entities. Chemical entities that associate with BTK may inhibit BTK, and are potential drug candidates. Alternatively, the structure encoded by the data may be displayed in a graphical three-dimensional representation on a computer screen. This allows visual inspection of the structure, as well as visual inspection of the structure's association with chemical entities.

Thus, according to another embodiment, the invention provides a method for designing, selecting and/or optimizing a chemical entity that binds to all or part of the molecule or molecular complex comprising the steps of:
(a) providing the structure coordinates of said molecule or molecular complex on a computer comprising the means for generating three-dimensional structural information of all or part of said molecule or molecular complex from said structure coordinates; and
(b) designing, selecting and/or optimizing said chemical entity by employing means for performing a fitting operation between said chemical entity and said three-dimensional structural information of all or part of said molecule or molecular complex.

In one embodiment, the method is for designing, selecting and or optimizing a chemical entity that binds with the binding pocket of a molecule or molecular complex. In one embodiment, the above method further comprises the following steps before step (a):
(c) producing a crystal of a molecule or molecular complex comprising BTK;
(d) determining the three-dimensional structure coordinates of the molecule or molecular complex by X-ray diffraction of the crystal; and
(e) identifying all or part of said binding pocket.

Three-dimensional structural information in step (a) may be generated by instructions such as a computer program or commands that can generate a three-dimensional structure or graphical representation; subtract distances between atoms; calculate chemical energies for a BTK molecule, molecular complex or homologs thereof; or calculate or minimize energies of an association of BTK molecule, molecular complex or homologs thereof to a chemical entity. These types of computer programs are known in the art. The graphical representation can be generated or displayed by commercially available software programs. Examples of software programs include but are not limited to QUANTA [Accelrys ©2001, 2002],O [Jones et al., Acta Crystallogr, .447, pp. 110-119 (1991)] and RIBBONS [Carson, J. Appl. Crystallogr., 24, pp. 9589-961 (1991)], which are incorporated herein by reference. Certain software programs may imbue this representation with physico-chemical attributes which are known from the chemical composition of the molecule, such as residue charge, hydrophobicity, torsional and rotational degrees of freedom for the residue or segment, etc. Examples of software programs for calculating chemical energies are described below.

Thus, according to another embodiment, the invention provides a method for evaluating the potential of a chemical entity to associate with all or part of a molecule or molecular complex as described previously in the different embodiments.

This method comprises the steps of: (a) employing computational means to perform a fitting operation between the chemical entity and all or part of the molecule or molecular complex described before; (b) analyzing the results of said fitting operation to quantify the association between the chemical entity and all or part of the molecule or molecular complex; and optionally (c) outputting said quantified association to a suitable output hardware, such as a CRT display terminal, a CD or DVD recorder, ZIP™ or JAZ™ drive, a disk drive, or other machine-readable data storage device, as described previously. The method may further comprise generating a three-dimensional structure, graphical representation thereof, or both of all or part of the molecule or molecular complex prior to step (a). In one embodiment, the method is for evaluating the ability of a chemical entity to associate with all or part of the binding pocket of a molecule or molecular complex.

In another embodiment, the invention provides a method for screening a plurality of chemical entities to associate at a deformation energy of binding of less than −7 kcal/mol with said binding pocket:
(a) employing computational means, which utilize said structure coordinates to perform a fitting operation between one of said chemical entities from the plurality of chemical entities and said binding pocket;
(b) quantifying the deformation energy of binding between the chemical entity and the binding pocket;
(c) repeating steps (a) and (b) for each remaining chemical entity; and
(d) outputting a set of chemical entities that associate with the binding pocket at a deformation energy of binding of less than −7 kcal/mol to a suitable output hardware.

In another embodiment, the method comprises the steps of:
(a) constructing a computer model of a binding pocket of the molecule or molecular complex;
(b) selecting a chemical entity to be evaluated by a method selected from the group consisting of assembling said chemical entity; selecting a chemical entity from a small molecule database; de novo ligand design of said chemical entity; and modifying a known agonist or inhibitor, or a portion thereof, of an BTK protein or homolog thereof;
(c) employing computational means to perform a fitting operation between computer models of said chemical entity to be evaluated and said binding pocket in order to provide an energy-minimized configuration of said chemical entity in the binding pocket; and
(d) evaluating the results of said fitting operation to quantify the association between said chemical entity and the binding pocket model, whereby evaluating the ability of said chemical entity to associate with said binding pocket.

In another embodiment, the invention provides a method of using a computer for evaluating the ability of a chemical entity to associate with all or part of the molecule or molecular complex, wherein said computer comprises a machine-readable data storage medium comprising a data storage material encoded with said structure coordinates defining said binding pocket and means for generating a three-dimensional graphical representation of the binding pocket, and wherein said method comprises the steps of:
(a) positioning a first chemical entity within all or part of said binding pocket using a graphical three-dimensional representation of the structure of the chemical entity and the binding pocket;
(b) performing a fitting operation between said chemical entity and said binding pocket by employing computational means;
(c) analyzing the results of said fitting operation to quantitate the association between said chemical entity and all or part of the binding pocket; and
(d) outputting said quantitated association to a suitable output hardware.

The above method may further comprise the steps of:
(e) repeating steps (a) through (d) with a second chemical entity; and
(f) selecting at least one of said first or second chemical entity that associates with all or part of said binding pocket based on said quantitated association of said first or second chemical entity.

Alternatively, the structure coordinates of the BTK binding pockets may be utilized in a method for identifying an agonist or antagonist of a molecule comprising a binding pocket of BTK. This method comprises the steps of:
(a) using a three-dimensional structure of the molecule or molecular complex to design or select a chemical entity;
(b) contacting the chemical entity with the molecule and molecular complex;
(c) monitoring the activity of the molecule or molecular complex; and
(d) classifying the chemical entity as an agonist or antagonist based on the effect of the chemical entity on the activity of the molecule or molecular complex.

In one embodiment, step (a) is using a three-dimensional structure of the binding pocket of the molecule or molecular complex. In another embodiment, the three-dimensional structure is displayed as a graphical representation.

In another embodiment, the method comprises the steps of:
(a) constructing a computer model of a binding pocket of the molecule or molecular complex;
(b) selecting a chemical entity to be evaluated by a method selected from the group consisting of assembling said chemical entity; selecting a chemical entity from a small molecule database; de novo ligand design of said chemical entity; and modifying a known agonist or inhibitor, or a portion thereof, of a BTK protein or homolog thereof;
(c) employing computational means to perform a fitting operation between computer models of said chemical entity to be evaluated and said binding pocket in order to provide an energy-minimized configuration of said chemical entity in the binding pocket; and
(d) evaluating the results of said fitting operation to quantify the association between said chemical entity and the binding pocket model, whereby evaluating the ability of said chemical entity to associate with said binding pocket;
(e) synthesizing said chemical entity; and
(f) contacting said chemical entity with said molecule or molecular complex to determine the ability of said compound to activate or inhibit said molecule.

In one embodiment, the invention provides a method of designing a compound or complex that associates with all or part of the binding pocket comprising the steps of:
(a) providing the structure coordinates of said binding pocket or protein on a computer comprising the means for generating three-dimensional structural information from said structure coordinates; and
(b) using the computer to perform a fitting operation to associate a first chemical entity with all or part of the binding pocket;
(c) performing a fitting operation to associate at least a second chemical entity with all or part of the binding pocket;
(d) quantifying the association between the first and second chemical entity and all or part of the binding pocket;
(e) optionally repeating steps (b) to (d) with at least one additional chemical entity, selecting a first, second and at least one additional chemical entity based on said quantified association of all of said first, second and at least one additional chemical entity;
(f) optionally, visually inspecting the relationship of the first, second and at least one additional chemical entity to each other in relation to the binding pocket on a computer screen using the three-dimensional graphical representation of the binding pocket and said first and second chemical entity; and
(g) assembling the first, second and at least one additional chemical entity into a compound or complex that associates with all or part of said binding pocket by model building.

For the first time, the present invention permits the use of molecular design techniques to identify, select and design chemical entities, including inhibitory compounds, capable of binding to BTK or BTK-like binding pockets, motifs and domains.

Applicants' elucidation of binding pockets on BTK provides the necessary information for designing new chemical entities and compounds that may interact with BTK substrate or BTK-like substrates, in whole or in part.

Throughout this section, discussions about the ability of a chemical entity to bind to, associate with or inhibit BTK binding pockets refer to features of the entity alone. Assays to determine if a compound binds to BTK are well known in the art and are exemplified below.

The design of compounds that bind to or inhibit BTK binding pockets according to this invention generally involves consideration of two factors. First, the chemical entity must be capable of physically and structurally associating with parts or all of the BTK binding pockets. Non-covalent molecular interactions important in this association include hydrogen bonding, van der Waals interactions, hydrophobic interactions and electrostatic interactions.

Second, the chemical entity must be able to assume a conformation that allows it to associate with the BTK binding pockets directly. Although certain portions of the chemical entity will not directly participate in these associations, those portions of the chemical entity may still influence the overall conformation of the molecule. This, in turn, may have a significant impact on potency. Such conformational requirements include the overall three-dimensional structure and orientation of the chemical entity in relation to all or a portion of the binding pocket, or the spacing between functional groups of a chemical entity comprising several chemical entities that directly interact with the BTK or BTK-like binding pockets.

The potential inhibitory or binding effect of a chemical entity on BTK binding pockets may be analyzed prior to its actual synthesis and testing by the use of computer modeling techniques. If the theoretical structure of the given entity suggests insufficient interaction and association between it and the BTK binding pockets, testing of the entity is obviated. However, if computer modeling indicates a strong interaction, the molecule may then be synthesized and tested for its ability to bind to an BTK binding pocket. This may be achieved by testing the ability of the molecule to inhibit BTK. In this manner, synthesis of inoperative compounds may be avoided.

A potential inhibitor of an BTK binding pocket may be computationally evaluated by means of a series of steps in which chemical entities or fragments are screened and selected for their ability to associate with the BTK binding pockets.

One skilled in the art may use one of several methods to screen chemical entities or fragments for their ability to associate with a BTK binding pocket. This process may begin by visual inspection of, for example, a BTK binding pocket on the computer screen based on the BTK structure coordinates or other coordinates which define a similar shape generated from the machine-readable storage medium. Selected fragments or chemical entities may then be positioned in a variety of orientations, or docked, within that binding pocket as defined supra. Docking may be accomplished using software such as QUANTA (Molecular Simulations, Inc., San Diego, Calif. © 1998, 2000) and Sybyl (Tripos Associates, St. Louis, Mo.), followed by energy minimization and molecular dynamics with standard molecular mechanics force fields, such as CHARMM and AMBER.

Specialized computer programs may also assist in the process of selecting fragments or chemical entities. These include:
1. GRID (P. J. Goodford, “A Computational Procedure for Determining Energetically Favorable Binding Sites on Biologically Important Macromolecules”, J. Med. Chem., 28, pp. 849-857 (1985)). GRID is available from Oxford University, Oxford, UK.
2. MCSS (A. Miranker et al., “Functionality Maps of Binding Sites: A Multiple Copy Simultaneous Search Method.” Proteins: Structure, Function and Genetics, 11, pp. 29-34 (1991)). MCSS is available from Molecular Simulations, San Diego, Calif.
3. AUTODOCK (D. S. Goodsell et al., “Automated Docking of Substrates to Proteins by Simulated Annealing”, Proteins: Structure, Function, and Genetics, 8, pp. 195-202 (1990)). AUTODOCK is available from Scripps Research Institute, La Jolla, Calif.
4. DOCK (I. D. Kuntz et al., “A Geometric Approach to Macromolecule-Ligand Interactions”, J. Mol. Biol., 161, pp. 269-288 (1982)). DOCK is available from University of California, San Francisco, Calif

Once suitable chemical, entities or fragments have been selected, they can be assembled into a single compound or complex. Assembly may be preceded by visual inspection of the relationship of the fragments to each other on the three-dimensional image displayed on a computer screen in relation to the structure coordinates of BTK. This would be followed by manual model building using software such as QUANTA (Molecular Simulations, Inc., San Diego, Calif. ©1998, 2000) or Sybyl (Tripos Associates, St. Louis, Mo.).

Useful programs to aid one of skill in the art in connecting the individual chemical entities or fragments include:
1. CAVEAT (P. A. Bartlett et al., “CAVEAT: A Program to Facilitate the Structure-Derived Design of Biologically Active Molecules”, in Molecular Recognition in Chemical and Biological Problems, Special Pub., Royal Chem. Soc., 78, pp. 182-196 (1989); G. Lauri and P. A. Bartlett, “CAVEAT: a Program to Facilitate the Design of Organic Molecules”, J. Comput. Aided Mol. Des., 8, pp. 51-66 (1994)). CAVEAT is available from the University of California, Berkeley, Calif.
2. 3D Database systems such as ISIS (MDL Information Systems, San Leandro, Calif). This area is reviewed in Y. C. Martin, “3D Database Searching in Drug Design”, J. Med. Chem, 35, pp. 2145-2154 (1992).
3. HOOK (M. B. Eisen et al., “HOOK: A Program for Finding Novel Molecular Architectures that Satisfy the Chemical and Steric Requirements of a Macromolecule Binding Site”, Proteins: Struct., Funct., Genet., 19, pp. 199- 221 (1994)). HOOK is available from Molecular Simulations, San Diego, Calif

Instead of proceeding to build an inhibitor of a BTK binding pocket in a step-wise fashion one fragment or chemical entity at a time as described above, inhibitory or other BTK binding compounds may be designed as a whole or “de novo” using either an empty binding pocket or optionally including some portion(s) of a known inhibitor(s). There are many de novo ligand design methods including:
1. LUDI (H.-J. Bohm, “The Computer Program LUDI: A New Method for the De Novo Design of Enzyme Inhibitors”, J. Comp. Aid. Molec. Design, 6, pp. 61-78 (1992)). LUDI is available from Molecular Simulations Incorporated, San Diego, Calif.
2. LEGEND (Y. Nishibata et al., Tetrahedron, 47, p. 8985 (1991)). LEGEND is available from Molecular Simulations Incorporated, San Diego, Calif.
3. LeapFrog (available from Tripos Associates, St. Louis, Mo.).
4. SPROUT (V. Gillet et al, “SPROUT: A Program for Structure Generation)”, J. Comput. Aided Mol. Design, 7, pp. 127-153 (1993)). SPROUT is available from the University of Leeds, UK.

Other molecular modeling techniques may also be employed in accordance with this invention (see, e.g, N. C. Cohen et al, “Molecular Modeling Software and Methods for Medicinal Chemistry”, J. Med. Chem., 33, pp. 883-894 (1990); see also, M. A. Navia and M. A. Murcko, “The Use of Structural Information in Drug Design”, Current Opinions in Structural Biology, 2, pp. 202-210 (1992); L. M. Balbes et. al., “A Perspective of Modern Methods in Computer-Aided Drug Design”, Reviews in Computational Chemistry, Vol. 5, K. B. Lipkowitz and D. B. Boyd, Eds, VCH, New York, pp. 337- 380 (1994); see also, W. C. Guida, “Software For Structure-Based Drug Design”, Curr. Opin. Struct. Biology, 4, pp. 777- 781 (1994)).

Once a chemical entity has been designed or selected by the above methods, the efficiency with which that chemical entity may bind to a BTK binding pocket may be tested and optimized by computational evaluation. For example, an effective BTK binding pocket inhibitor must preferably demonstrate a relatively small difference in energy between its bound and free states (i.e, a small deformation energy of binding). Thus, the most efficient BTK binding pocket inhibitors should preferably be designed with a deformation energy of binding of not greater than about 10 kcal/mole, more preferably, not greater than 7 kcal/mole. BTK binding pocket inhibitors may interact with the binding pocket in more than one conformation that is similar in overall binding energy. In those cases, the deformation energy of binding is taken to be the difference between the energy of the free chemical entity and the average energy of the conformations observed when the inhibitor binds to the protein.

A chemical entity designed or selected as binding to a BTK binding pocket may be further computationally optimized so that in its bound state it would preferably lack repulsive electrostatic interaction with the target enzyme and with the surrounding water molecules. Such non-complementary electrostatic interactions include repulsive charge-charge, dipole-dipole and charge-dipole interactions.

Specific computer software is available in the art to evaluate compound deformation energy and electrostatic interactions. Examples of programs designed for such uses include: Gaussian 94, revision C (M. J. Frisch, Gaussian, Inc., Pittsburgh, Pa. ©1995); AMBER, version 4.1 (P. A. Kollman, University of California at San Francisco, 01995); QUANTA/CHARM:NI (Molecular Simulations, Inc., San Diego, Calif. 01998, 2000); Insight II/Discover (Molecular Simulations, Inc., San Diego, Calif. 01998); DelPhi (Molecular Simulations, Inc., San Diego, Calif. 01998); and AMSOL (Quantum Chemistry Program Exchange, Indiana University). These programs may be implemented, for instance, using a Silicon Graphics workstation such as an Indigo2 with “IMPACT” graphics. Other hardware systems and software packages will be known to those skilled in the art.

Another approach enabled by this invention, is the computational screening of small molecule databases for chemical entities or compounds that can bind in whole, or in part, to a BTK binding pocket. In this screening, the quality of fit of such entities to the binding pocket may be judged either by shape complementarity or by estimated interaction energy (E. C. Meng et al, J. Comp. Chem., 13, pp. 505-524 (1992)).

According to another embodiment, the invention provides compounds which associate with a BTK binding pocket produced or identified by the method set forth above.

Another particularly useful drug design technique enabled by this invention is iterative drug design. Iterative drug design is a method for optimizing associations between a protein and a compound by determining and evaluating the three-dimensional structures of successive sets of protein/compound complexes.

In iterative drug design, crystals of a series of protein or protein complexes are obtained and then the three-dimensional structures of each crystal is solved.

Such an approach provides insight into the association between the proteins and compounds of each complex. This is accomplished by selecting compounds with inhibitory activity, obtaining crystals of this new protein/compound complex, solving the three-dimensional structure of the complex, and comparing the associations between the new protein/compound complex and previously solved protein/compound complexes. By observing how changes in the compound affected the protein/compound associations, these associations may be optimized.

In some cases, iterative drug design is carried out by forming successive protein-compound complexes and then crystallizing each new complex. High throughput crystallization assays may be used to find a new crystallization condition or to optimize the original protein or complex crystallization condition for the new complex. Alternatively, a pre-formed protein crystal may be soaked in the presence of an inhibitor, thereby forming a protein/compound complex and obviating the need to crystallize each individual protein/compound complex.

The invention provides a data storage medium which comprises the structure coordinates of molecules or molecular complexes of the BTK binding pockets. In one aspect, the data storage medium comprises the structure coordinates of the binding pocket. The invention also provides a computer comprising the data storage medium. Such storage medium when read and utilized by a computer programmed with appropriate software can display, on a computer screen or similar viewing device, a three-dimensional graphical representation of such binding pockets.

The invention also provides methods for designing, evaluating and identifying compounds which bind to the molecules or molecular complexes or their binding pockets. Such compounds are potential inhibitors of BTK or its homologues.

The invention also provides a method for determining at least a portion of the three-dimensional structure of molecules or molecular complexes which contain at least some structurally similar features to BTK. This is achieved by using at least some of the structure coordinates obtained from the BTK protein or protein complexes.

Some aspects of the invention are directed to a crystalline composition comprising SEQ ID NO:3, and a ligand, wherein said crystalline composition is characterized with space group p 2 21 21 and has unit cell parameters a=38.155±2Å; b=72.394±2Å; c=103.946±2Å; a=90°; b=90°; g=90°. In some aspects, the ligand is a compound of Formula (I):

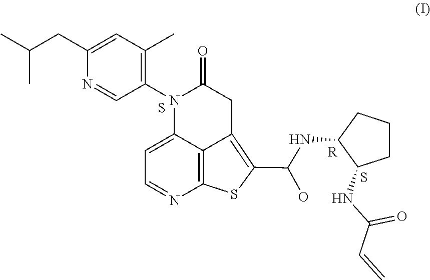

or pharmaceutically acceptable salts, hydrates, polymorphs or solvates thereof

Some aspects are directed to methods for identifying a candidate inhibitor of BTK, wherein said method comprises: generating a three-dimensional structure of a binding site of BTK on a computer, wherein the three dimensional structure coordinates possess the unit cell and space group parameters of the crystalline composition comprising SEQ ID NO:3, and Compound I, employing said three dimensional structure to design or select a candidate inhibitor; and contacting said candidate inhibitor with human BTK and measuring the ability of said candidate inhibitor to bind to BTK. In some aspects, the candidate inhibitor makes a direct covalent bond with Cys481. In some aspects, the candidate inhibitor makes a hydrogen bond with Lys430. In some aspects, the candidate inhibitor makes a hydrogen bond with Met477. In some aspects, the candidate inhibitor makes a hydrogen bond with Asp539.

Some aspects are directed to methods for identifying and/or designing a candidate inhibitor using a human BTK crystal comprising a human BTK protein, wherein said method comprises:
a) preparing the crystalline composition of comprising SEQ ID NO:3, and Compound I; b) soaking another candidate inhibitor into the crystalline composition displacing the original ligand to form a inhibitor-crystal complex; c) determining the three-dimensional structure coordinates of the inhibitor-crystal complex prepared in step b); d)using the structure coordinates from step c) to design and/or identifying a candidate inhibitor; and e) contacting said candidate inhibitor with human BTK and measuring the ability of said candidate inhibitor to bind to BTK.

Some aspects are directed to methods of designing a compound or complex that interacts with a binding pocket or domain selected from the group comprising (i) BTK amino acid residues Leu408, Gly409, Thr410, Gly411, Va1416, Ala428, Lys430, Asn439, Met449, Leu452, Va1458, Ile472, Thr474, Glu475, Tyr476, Met477, Gly480, Cys481, Asn 484, Arg525, Leu528, Ser538, Asp539, and Phe540 according to Table 2; and (ii) BTK amino acid residues according to Table 2; comprising the steps of:

(a) producing a crystal of BTK in complex with a compound of Formula (I):

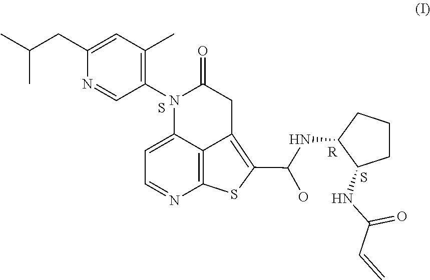

or pharmaceutically acceptable salts, hydrates, polymorphs or solvates thereof, wherein said BTI. comprises SIE) ID NO: 3, wherein said crystal is characterized with space group p 2 21 21 and has unit cell parameters a=38.155±2Å; b=72.394±2Å; c=103.946±2Å; a=90°; b=90°; g=90°;
(b) providing the structure coordinates of said binding pocket or domain of the crystal of step (a), according to Table 2, on a computer capable of generating three-dimensional structural information from said structure coordinates, wherein the root mean square deviation of the backbone atoms is not greater than about 2.5 Å;
(c) using the computer to dock a first chemical entity in part of the binding pocket or domain;
(d) docking at least a second chemical entity in another part of the binding pocket or domain;
(e) quantifying the association between the first or second chemical entity and part of the binding pocket or domain;
(f) repeating steps (c) to (e) with at least one additional chemical entity, selecting a first, second and at least one additional chemical entity based on said quantified association of all of said first, second and at least one additional chemical entity;
(g) optionally, visually inspecting the relationship of the first, second and at least one additional chemical entity to each other in relation to the binding pocket or domain on a computer screen using the three-dimensional graphical representation of the binding pocket or domain and said first, second and at least one additional chemical entity; and
(h) assembling the first, second and at least one additional chemical entity into a compound or complex that interacts with said binding pocket or domain by model building.

Some aspects are directed to methods for identifying a candidate inhibitor that interacts with a binding site of a BTK protein, comprising the steps of:
(a) producing a crystal of BTK in complex with a compound of Formula (I):

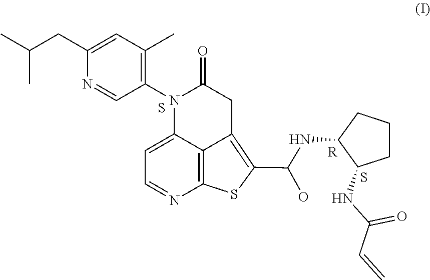

or pharmaceutically acceptable salts, hydrates, polymorphs or solvates thereof, wherein said BTK comprises SEQ ID NO: 3, wherein the crystal is characterized with space group p 2 21 21 and has unit cell parameters a=38.155±2Å; b=72.394±2Å; c=103.946±2Å; a=90°; b=90°; g=90°;
(b) obtaining the structure coordinates of amino acids of the crystal of step (a) according to Table 2;
(c) generating a three-dimensional model of said BTK protein using the structure coordinates of the amino acids generated in step (b), wherein the root mean square deviation from backbone atoms is not more than ±2.0 Å;
(d) determining a binding site of said human BTK protein from said three-dimensional model; and
(e) performing computer fitting analysis to identify the candidate inhibitor which interacts with said binding site.

Some aspects further comprise the step of:
(f) contacting the identified candidate inhibitor with said BTK protein in order to determine the effect of the inhibitor on BTK activity.

In some aspects, the binding site of said BTK protein determined in step (d) comprises the structure coordinates, according to Table 2, of BTK amino acid residues Leu408, Gly409, Thr410, Gly411, Va1416, Ala428, Lys430, Asn439, Met449, Leu452, Va1458, Ile472, Thr474, Glu475, Tyr476, Met477, Gly480, Cys481, Asn 484, Arg525, Leu528, Ser538, Asp539, and Phe540, wherein the root mean square deviation is not more than ±2.0 Å.

Some aspects are directed to methods of using a crystal comprising a BTK binding pocket or domain in an inhibitor screening assay comprising the steps of:
(a) producing a crystal of BTK in complex with a compound of Formula (I):

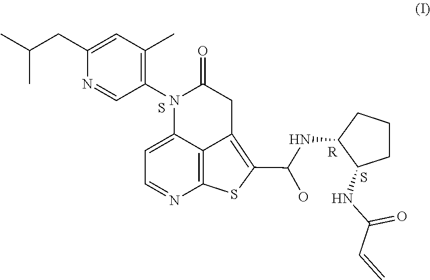

or pharmaceutically acceptable salts, hydrates, polymorphs or solvates thereof, wherein said BTK comprises SEQ ID NO: 3, wherein said crystal is characterized with space group p 2 21 21 and has unit cell parameters a=38.155±2Å; b=72.394±2Å; c=103.946±2Å; a=90°; b=90°; g=90°;
(b) providing the structure coordinates of said binding pocket or domain of the crystal of step (a), according to Table 2, on a computer capable of generating three-dimensional structural information from said structure coordinates;
(c) selecting a potential inhibitor by performing rational drug design with a three-dimensional structure determined for the crystal, wherein said selecting is performed in conjunction with computer modeling;
(d) contacting the potential inhibitor with the kinase; and
(e) detecting the ability of the potential inhibitor for inhibiting the kinase's enzymatic activity.

Some aspects are drawn to methods of designing a compound or complex that interacts with a BTK binding pocket or domain comprising the steps of:
(a) producing a crystal of human BTK in complex with a compound of Formula (I):

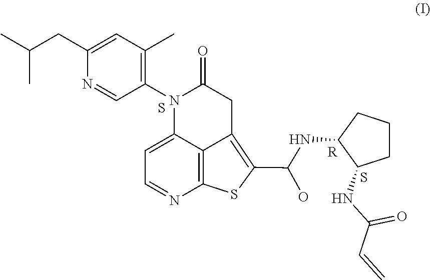

or pharmaceutically acceptable salts, hydrates, polymorphs or solvates thereof, wherein said BTK comprises SEQ ID NO^{.} 3, wherein said crystal is characterized with space group p 2 21 21 and has unit cell parameters a=38.155±2Å; b=72.394±2Å; c=103.946±2Å; a=90°; b=90°; g=90°;
(b) providing the structure coordinates of said binding pocket or domain of the crystal of step (a), according to Table 2, on a computer capable of generating three-dimensional structural information from said structure coordinates;
(c) using the computer to dock a first chemical entity in part of the binding pocket or domain;
(d) docking at least a second chemical entity in another part of the binding pocket or domain;
(e) quantifying the association between the first or second chemical entity and part of the binding pocket or domain;
(f) repeating steps (c) to (e) with at least one additional chemical entity, selecting a first, second and at least one additional chemical entity based on said quantified association of all of said first, second and at least one additional chemical entity;
(g) optionally, visually inspecting the relationship of the first, second and at least one additional chemical entity to each other in relation to the binding pocket or domain on a computer screen using the three-dimensional graphical representation of the binding pocket or domain and said first, second and at least one additional chemical entity; and
(h) assembling the first, second and at least one additional chemical entity into a compound or complex that interacts with said binding pocket or domain by model building.

Some aspects are directed to methods for identifying a candidate inhibitor that interacts with a binding site of a BTK protein comprising the steps of:
(a) producing a crystal of BTK in complex with a compound of Formula (I):

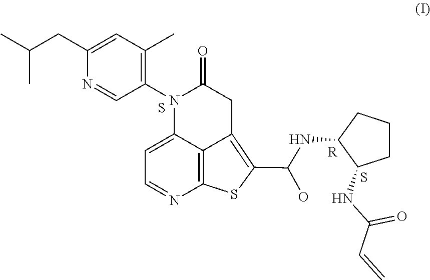

or pharmaceutically acceptable salts, hydrates, polymorphs or solvates thereof, wherein said BTK comprises SEQ ID NO: 3, wherein said crystal is characterized with space group p 2 21 21 and has unit cell parameters a=38.155±2Å; b=72.394±2Å; c=103.946±2Å; a=90°; b=90°; g=90°;
(b) determining the three-dimensional structure coordinates of BTK using the crystal in step (a);
(c) using the structure coordinates from step (b) to generate a three-dimensional structure of the crystallized BTK protein produced in step (a);
(d) employing said three-dimensional structure to design or select said candidate inhibitor;
(e) synthesizing said candidate inhibitor; and
(f) contacting said candidate inhibitor with human BTK protein to determine the ability of said candidate inhibitor to bind to human BTK.

Some aspects are directed to methods for identifying a candidate inhibitor that interacts with a binding site of a human BTK protein comprising the steps of:
(a) producing a crystal of BTK in complex with a compound of Formula (I):

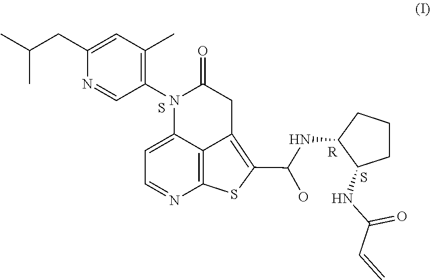

or pharmaceutically acceptable salts, hydrates, polymorphs or solvates thereof, wherein said BTK comprises SEQ ID NO: 3, wherein said crystal is characterized with space group p 2 21 21 and has unit cell parameters a=38.155±2Å; b=72.394±2Å; c=103.946±2Å; a=90°; b=90°; g=90°;
(b) determining the three-dimensional structure coordinates of BTK using the crystal in step (a);
(c) using the structure coordinates from step (b) to generate a three-dimensional structure of a molecular complex comprising a binding site of amino acid residues Leu408, Gly409, Thr410, Gly411, Va1416, Ala428, Lys430, Asn439, Met449, Leu452, Va1458, Ile472, Thr474, Glu475, Tyr476, Met477, Gly480, Cys481, Asn 484, Arg525, Leu528, Ser538, Asp539, and Phe540 according to Table 2, wherein the root mean square deviation of the backbone atoms is not greater than about 2.5 Å;
(d) employing said three-dimensional structure to design or select said candidate inhibitor;
(e) synthesizing said candidate inhibitor; and
(f) contacting said candidate inhibitor with BTK to determine the ability of said candidate inhibitor to bind to BTK.

Some aspects of the invention are directed to a crystalline composition consisting essentially of SEQ ID NO:3, and a ligand, wherein said crystalline composition is characterized with space group p 2 21 21 and has unit cell parameters a=38.155±2Å; b=72.394±2Å; c=103.946±2Å; a=90°; b=90°; g=90°. In some aspects, the ligand is a compound of Formula

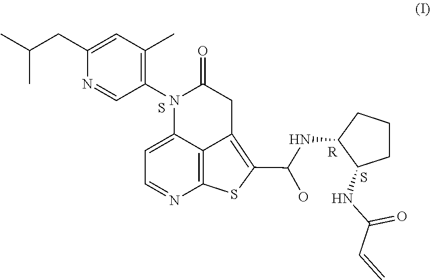

or pharmaceutically acceptable salts, hydrates, polymorphs or solvates thereof

Some aspects are directed to methods for identifying a candidate inhibitor of BTK, wherein said method comprises: generating a three-dimensional structure of a binding site of BTK on a computer, wherein the three dimensional structure coordinates possess the unit cell and space group parameters of the crystalline composition comprising SEQ ID NO:3, and Compound I, employing said three dimensional structure to design or select a candidate inhibitor; and contacting said candidate inhibitor with human BTK and measuring the ability of said candidate inhibitor to bind to BTK. In some aspects, the candidate inhibitor makes a direct covalent bond with Cys481. In some aspects, the candidate inhibitor makes a hydrogen bond with Lys430. In some aspects, the candidate inhibitor makes a hydrogen bond with Met477. In some aspects, the candidate inhibitor makes a hydrogen bond with Asp539.

Some aspects are directed to methods for identifying and/or designing a candidate inhibitor using a human BTK crystal comprising a human BTK protein, wherein said method comprises:
a) preparing the crystalline composition of consisting essentially of SEQ ID NO:3, and the compound of Formula (I) and b) soaking another candidate inhibitor into the crystalline composition, displacing the compound of Formula (I) (original ligand) forming an inhibitor-crystal complex; c) determining the three-dimensional structure coordinates of the inhibitor-crystal complex prepared in step b); d) using the structure coordinates from step c) to design or select a candidate inhibitor; and e) contacting said candidate inhibitor with human BTK and measuring the ability of said candidate inhibitor to bind to BTK.

Some aspects are directed to methods of designing a compound or complex that interacts with a binding pocket or domain selected from the group consisting essentially of (i) BTK amino acid residues Leu408, Gly409, Thr410, Gly411, Va1416, Ala428, Lys430, Asn439, Met449, Leu452, Va1458, Ile472, Thr474, Glu475, Tyr476, Met477, Gly480, Cys481, Asn 484, Arg525, Leu528, Ser538, Asp539, and Phe540 according to Table 2; and (ii) BTK amino acid residues according to Table 2; comprising the steps of:
(a) producing a crystal of BTK in complex with a compound of Formula (I):

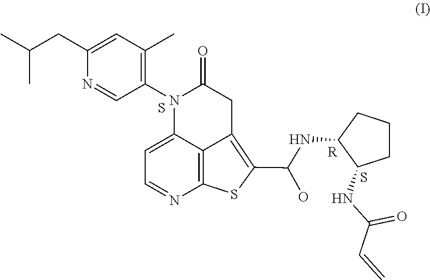

or pharmaceutically acceptable salts, hydrates, polymorphs or solvates thereof, wherein said BTK consists essentially of SEQ ID NO: 3, wherein said crystal is characterized with space group p 2 21 21 and has unit cell parameters a=38.155±2Å; b=72.394±2Å; c=103.946±2Å; a=90°; b=90°; g=90°;
(b) providing the structure coordinates of said binding pocket or domain of the crystal of step (a), according to Table 2, on a computer capable of generating three-dimensional structural information from said structure coordinates, wherein the root mean square deviation of the backbone atoms is not greater than about 2.5 Å;
(c) using the computer to dock a first chemical entity in part of the binding pocket or domain;
(d) docking at least a second chemical entity in another part of the binding pocket or domain;
(e) quantifying the association between the first or second chemical entity and part of the binding pocket or domain;
(f) repeating steps (c) to (e) with at least one additional chemical entity, selecting a first, second and at least one additional chemical entity based on said quantified association of all of said first, second and at least one additional chemical entity;
(g) optionally, visually inspecting the relationship of the first, second and at least one additional entity to each other in relation to the binding pocket or domain on a computer screen using the three-dimensional graphical representation of the binding pocket or domain and said first, second and at least one additional entity; and
(h) assembling the first, second and at least one additional entity into a compound or complex that interacts with said binding pocket or domain by model building.

Some aspects are directed to methods for identifying a candidate inhibitor that interacts with a binding site of a BTK protein, comprising the steps of:
(a) producing a crystal of BTK in complex with a compound of Formula (I):

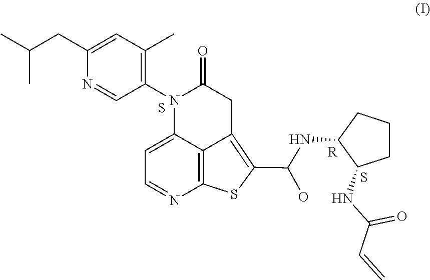

or pharmaceutically acceptable salts, hydrates, polymorphs or solvates thereof, wherein said BTK consists essentially of SEQ ID NO: 3, wherein the crystal is characterized with space group p 2 21 21 and has unit cell parameters a=38.155±2Å; b=72.394±2Å; c=103.946±2Å; a=90°; b=90°; g=90°;
(b) obtaining the structure coordinates of amino acids of the crystal of step (a) according to Table 2;
(c) generating a three-dimensional model of said BTK protein using the structure coordinates of the amino acids generated in step (b), wherein the root mean square deviation from backbone atoms is not more than ±2.0 Å;
(d) determining a binding site of said BTK protein from said three-dimensional model; and
(e) performing computer fitting analysis to identify the candidate inhibitor which interacts with said binding site.

Some aspects further comprise the step of:
(f) contacting the identified candidate inhibitor with said BTK protein in order to determine the effect of the inhibitor on BTK activity.

In some aspects, the binding site of said BTK protein determined in step (d) consists essentially of the structure coordinates, according to Table 2, of BTK amino acid residues Leu408, Gly409, Thr410, Gly411, Va1416, Ala428, Lys430, Asn439, Met449, Leu452, Va1458, Ile472, Thr474, Glu475, Tyr476, Met477, Gly480, Cys481, Asn 484, Arg525, Leu528, Ser538, Asp539, and Phe540, wherein the root mean square deviation is not more than ±2.0 Å.

Some aspects are directed to methods of using a crystal consisting essentially of a BTK binding pocket or domain in an inhibitor screening assay comprising the steps of:
(a) producing a crystal of BTK in complex with a compound of Formula (I):

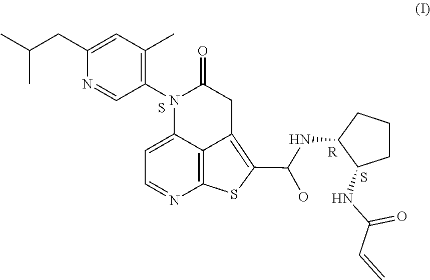

or pharmaceutically acceptable salts, hydrates, polymorphs or solvates thereof, wherein said BTK consists essentially of SEQ ID NO: 3, wherein said crystal is characterized with space group p 2 21 21 and has unit cell parameters a=38.155±2Å; b=72.394±2Å; c=103.946±2Å; a=90°; b=90°; g=90°;
(b) providing the structure coordinates of said binding pocket or domain of the crystal of step (a), according to Table 2, on a computer capable of generating three-dimensional structural information from said structure coordinates;
(c) selecting a potential inhibitor by performing rational drug design with a three-dimensional structure determined for the crystal, wherein said selecting is performed in conjunction with computer modeling;
(d) contacting the potential inhibitor with the kinase; and
(e) detecting the ability of the potential inhibitor for inhibiting the kinase's enzymatic activity.

Some aspects are drawn to methods of designing a compound or complex that interacts with a BTK binding pocket or domain comprising the steps of:
(a) producing a crystal of human BTK in complex with a compound of Formula (I):

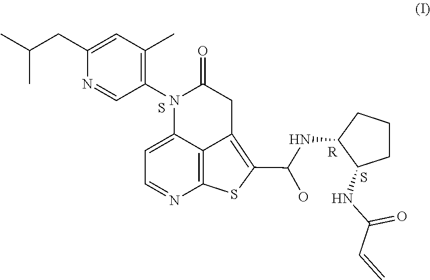

or pharmaceutically acceptable salts, hydrates, polymorphs or solvates thereof, wherein said BTK consists essentially of SE( )ID NO: 3, wherein said crystal is characterized with space group p 2 21 21 and has unit cell parameters a=38.155±2Å; b=72.394±2Å; c=103.946±2Å; a=90°; b=90°; g=90°;
(b) providing the structure coordinates of said binding pocket or domain of the crystal of step (a), according to Table 2, on a computer capable of generating three-dimensional structural information from said structure coordinates;
(c) using the computer to dock a first chemical entity in part of the binding pocket or domain;
(d) docking at least a second chemical entity in another part of the binding pocket or domain;
(e) quantifying the association between the first or second chemical entity and part of the binding pocket or domain;
(f) repeating steps (c) to (e) with at least one additional chemical entity, selecting a first, second and at least one additional chemical entity based on said quantified association of all of said first, second and at least one additional chemical entity;
(g) optionally, visually inspecting the relationship of the first, second and at least one additional chemical entity to each other in relation to the binding pocket or domain on a computer screen using the three-dimensional graphical representation of the binding pocket or domain and said first, second and at least one additional chemical entity; and
(h) assembling the first, second and at least one additional chemical entity into a compound or complex that interacts with said binding pocket or domain by model building.

Some aspects are directed to methods for identifying a candidate inhibitor that interacts with a binding site of a BTK protein comprising the steps of:
(a) producing a crystal of BTK in complex with a compound of Formula (I):

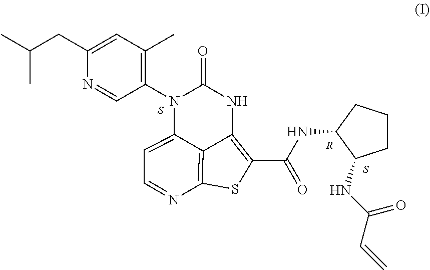

or pharmaceutically acceptable salts, hydrates, polymorphs or solvates thereof, wherein said BTK consisting essentially of SEQ ID NO: 3, wherein said crystal is characterized with space group p 2 21 21 and has unit cell parameters a=38.155±2Å; b=72.394±2Å; c=103.946±2Å; a=90°; b=90°; g=90°;
(b) determining the three-dimensional structure coordinates of BTK using the crystal in step (a);
(c) using the structure coordinates from step (b) to generate a three-dimensional structure of the crystallized BTK protein produced in step (a);
(d) employing said three-dimensional structure to design or select said candidate inhibitor;
(e) synthesizing said candidate inhibitor; and
(f) contacting said candidate inhibitor with human BTK protein to determine the ability of said candidate inhibitor to bind to human BTK.

Some aspects are directed to methods for identifying a candidate inhibitor that interacts with a binding site of a human BTK protein comprising the steps of:
(a) producing a crystal of BTK in complex with a compound of Formula (I):

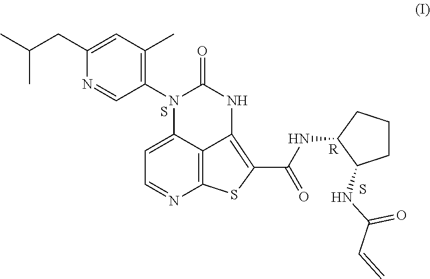

or pharmaceutically acceptable salts, hydrates, polymorphs or solvates thereof, wherein said BTK consisting essentially of SEQ ID NO: 3, wherein said crystal is characterized with space group p 2 21 21 and has unit cell parameters a=38.155±2Å; b=72.394±2Å; c=103.946±2Å; a=90°; b=90°; g=90°;
(b) determining the three-dimensional structure coordinates of BTK using the crystal in step (a);
(c) using the structure coordinates from step (b) to generate a three-dimensional structure of a molecular complex consisting essentially of a binding site of amino acid residues Leu408, Gly409, Thr410, Gly411, Va1416, Ala428, Lys430, Asn439, Met449, Leu452, Va1458, Ile472, Thr474, Glu475, Tyr476, Met477, Gly480, Cys481, Asn 484, Arg525, Leu528, Ser538, Asp539, and Phe540 according to Table 2, wherein the root mean square deviation of the backbone atoms is not greater than about 2.5 Å;
(d) employing said three-dimensional structure to design or select said candidate inhibitor;
(e) synthesizing said candidate inhibitor; and
(f) contacting said candidate inhibitor with BTK to determine the ability of said candidate inhibitor to bind to BTK.

Some aspects of the invention are directed to a crystalline composition consisting essentially of SEQ ID NO:3, and a ligand, wherein said crystalline composition is characterized with space group p 2 21 21 and has unit cell parameters a=38.155±2Å; b=72.394±2Å; c=103.946±2Å; a=90°; b=90°; g=90°. In some aspects, the ligand is a compound of Formula (I):

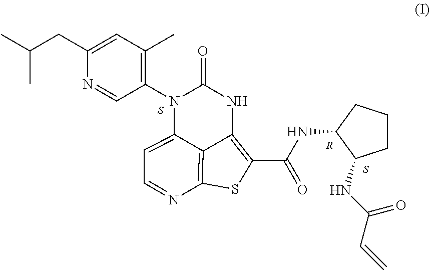

or pharmaceutically acceptable salts, hydrates, polymorphs or solvates thereof.

Some aspects are directed to methods for identifying a candidate inhibitor of BTK, wherein said method comprises: generating a three-dimensional structure of a binding site of BTK on a computer, wherein the three dimensional structure coordinates possess the unit cell and space group parameters of the crystalline composition consisting of SEQ ID NO:3, and Compound I, employing said three dimensional structure to design or select a candidate inhibitor; and contacting said candidate inhibitor with human BTK and measuring the ability of said candidate inhibitor to bind to BTK. In some aspects, the candidate inhibitor makes a direct covalent bond with Cys481. In some aspects, the candidate inhibitor makes a hydrogen bond with Lys430. In some aspects, the candidate inhibitor makes a hydrogen bond with Met477. In some aspects, the candidate inhibitor makes a hydrogen bond with Asp539.

Some aspects are directed to methods for identifying and or designing a candidate inhibitor using a human BTK crystal comprising a human BTK protein, wherein said method comprises:
a) preparing the crystalline composition of consisting of SEQ ID NO:3, and compound of Formula (I) and b) soaking another candidate inhibitor into the crystalline composition, displacing the compound of Formula (I) (original ligand) to form a inhibitor-crystal complex; c) determining the three-dimensional structure coordinates of the inhibitor-crystal complex prepared in step b); d) using the structure coordinates from step c) to design or select a candidate inhibitor; and e) contacting said candidate inhibitor with human BTK and measuring the ability of said candidate inhibitor to bind to BTK.

Some aspects are directed to methods of designing a compound or complex that interacts with a binding pocket or domain selected from the group consisting of (i) BTK amino acid residues Leu408, Gly409, Thr410, Gly411, Va1416, Ala428, Lys430, Asn439, Met449, Leu452, Va1458, Ile472, Thr474, Glu475, Tyr476, Met477, Gly480, Cys481, Asn 484, Arg525, Leu528, Ser538, Asp539, and Phe540 according to Table 2; and (ii) BTK amino acid residues according to Table 2; comprising the steps of:
(a) producing a crystal of BTK in complex with a compound of Formula (I):

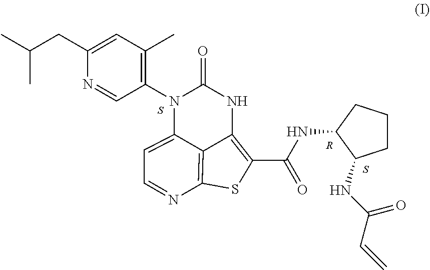

or pharmaceutically acceptable salts, hydrates, polymorphs or solvates thereof, wherein said BTK consisting of SEQ ID NO: I wherein said crystal is characterized with space group p 2 21 21 and has unit cell parameters a=38.155±2Å; b=72.394±2Å; c=103.946±2Å; a=90°; b=90°; g=90°;
(b) providing the structure coordinates of said binding pocket or domain of the crystal of step (a), according to Table 2, on a computer capable of generating three-dimensional structural information from said structure coordinates, wherein the root mean square deviation of the backbone atoms is not greater than about 2.5 Å;
(c) using the computer to dock a first chemical entity in part of the binding pocket or domain;
(d) docking at least a second chemical entity in another part of the binding pocket or domain;
(e) quantifying the association between the first or second chemical entity and part of the binding pocket or domain;
(f) repeating steps (c) to (e) with at least one additional chemical entity, selecting a first, second and at least one additional chemical entity based on said quantified association of all of said first, second and at least one additional chemical entity;
(g) optionally, visually inspecting the relationship of the first, second and at least one additional chemical entity to each other in relation to the binding pocket or domain on a computer screen using the three-dimensional graphical representation of the binding pocket or domain and said first, second and at least one additional chemical entity; and
(h) assembling the first, second and at least one additional chemical entity into a compound or complex that interacts with said binding pocket or domain by model building.

Some aspects are directed to methods for identifying a candidate inhibitor that interacts with a binding site of a BTK protein, comprising the steps of:
(a) producing a crystal of BTK in complex with a compound of Formula (I):

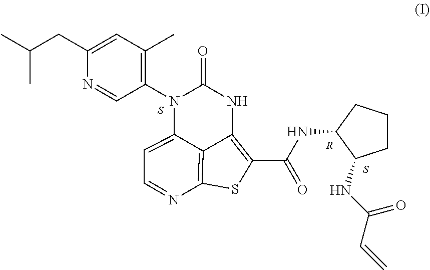

or pharmaceutically acceptable salts, hydrates, polymorphs or solvates thereof, wherein said BTK consisting of SEQ ID NO: 3, wherein the crystal is characterized with space group p 2 21 21 and has unit cell parameters a=38.155±2Å; b=72.394±2Å; c=103.946±2Å; a=90°; b=90°; g=90°;
(b) obtaining the structure coordinates of amino acids of the crystal of step (a) according to Table 2;
(c) generating a three-dimensional model of said BTK protein using the structure coordinates of the amino acids generated in step (b), wherein the root mean square deviation from backbone atoms is not more than ±2.0 Å;
(d) determining a binding site of said human BTK protein from said three-dimensional model; and
(e) performing computer fitting analysis to identify the candidate inhibitor which interacts with said binding site.

Some aspects further comprise the step of:
(f) contacting the identified candidate inhibitor with said BTK protein in order to determine the effect of the inhibitor on BTK activity.

In some aspects, the binding site of said BTK protein determined in step (d) consists essentially of the structure coordinates, according to Table 2, of BTK amino acid residues Leu408, Gly409, Thr410, Gly411, Va1416, Ala428, Lys430, Asn439, Met449, Leu452, Va1458, Ile472, Thr474, Glu475, Tyr476, Met477, Gly480, Cys481, Asn 484, Arg525, Leu528, Ser538, Asp539, and Phe540, wherein the root mean square deviation is not more than ±2.0 Å.

Some aspects are directed to methods of using a crystal consisting a BTK binding pocket or domain in an inhibitor screening assay comprising the steps of:
(a) producing a crystal of BTK in complex with a compound of Formula (I):

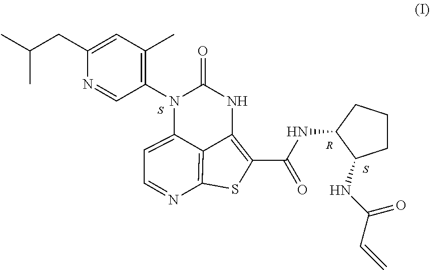

or pharmaceutically acceptable salts, hydrates, polymorphs or solvates thereof, wherein said BTK consists of SEQ ID NO: 3, wherein said crystal is characterized with space group p 2 21 21 and has unit cell parameters a=38.155±2Å; b=72.394±2Å; c=103.946±2Å; a=90°; b=90°; g=90°;
(b) providing the structure coordinates of said binding pocket or domain of the crystal of step (a), according to Table 2, on a computer capable of generating three-dimensional structural information from said structure coordinates;
(c) selecting a potential inhibitor by performing rational drug design with a three-dimensional structure determined for the crystal, wherein said selecting is performed in conjunction with computer modeling;
(d) contacting the potential inhibitor with the kinase; and
(e) detecting the ability of the potential inhibitor for inhibiting the kinase's enzymatic activity.

Some aspects are drawn to methods of designing a compound or complex that interacts with a BTK binding pocket or domain comprising the steps of:
(a) producing a crystal of human BTK in complex with a compound of Formula (I):

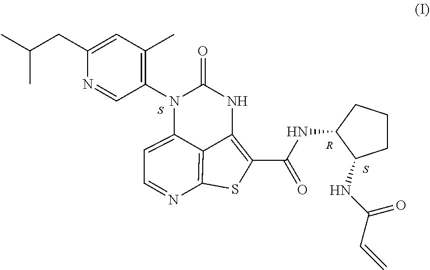

or pharmaceutically acceptable salts, hydrates, polymorphs or solvates thereof, wherein said BTK consists of SEQ ID NO: 3, wherein said crystal is characterized with space group p 2 21 21 and has unit cell parameters a=38.155±2Å; b=72.394±2Å; c=103.946±2Å; a=90°; b=90°; g=90°;
(b) providing the structure coordinates of said binding pocket or domain of the crystal of step (a), according to Table 2, on a computer capable of generating three-dimensional structural information from said structure coordinates;
(c) using the computer to dock a first chemical entity in part of the binding pocket or domain;
(d) docking at least a second chemical entity in another part of the binding pocket or domain;
(e) quantifying the association between the first or second chemical entity and part of the binding pocket or domain;
(f) repeating steps (c) to (e) with at least one additional chemical entity, selecting a first, second and at least one additional chemical entity based on said quantified association of all of said first, second and at least one additional chemical entity;
(g) optionally, visually inspecting the relationship of the first, second and at least one additional chemical entity to each other in relation to the binding pocket or domain on a computer screen using the three-dimensional graphical representation of the binding pocket or domain and said first, second and at least one additional chemical entity; and
(h) assembling the first, second and at least one additional chemical entity into a compound or complex that interacts with said binding pocket or domain by model building.

Some aspects are directed to methods for identifying a candidate inhibitor that interacts with a binding site of a BTK protein comprising the steps of:
(a) producing a crystal of BTK in complex with a compound of Formula (I):

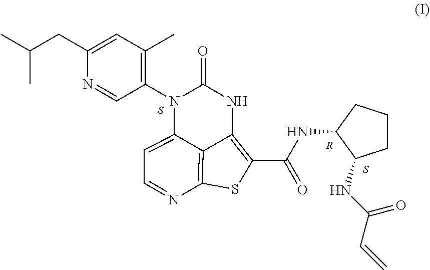

or pharmaceutically acceptable salts, hydrates, polymorphs or solvates thereof, wherein said BTK consisting of SEQ ID NO: 3, wherein said crystal is characterized with space group p 2 21 21 and has unit cell parameters a=38.155±2Å; b=72.394±2Å; c=103.946±2Å; a=90°; b=90°; g=90°;
(b) determining the three-dimensional structure coordinates of BTK using the crystal in step (a);
(c) using the structure coordinates from step (b) to generate a three-dimensional structure of the crystallized BTK protein produced in step (a);
(d) employing said three-dimensional structure to design or select said candidate inhibitor;
(e) synthesizing said candidate inhibitor; and
(f) contacting said candidate inhibitor with human BTK protein to determine the ability of said candidate inhibitor to bind to human BTK.

Some aspects are directed to methods for identifying a candidate inhibitor that interacts with a binding site of a human BTK protein comprising the steps of:
(a) producing a crystal of BTK in complex with a compound of Formula (I):

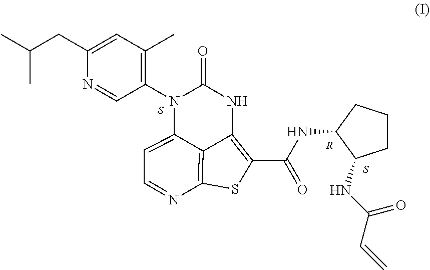

or pharmaceutically acceptable salts, hydrates, polymorphs or solvates thereof, wherein said BTK consisting of SEQ ID NO: 3, wherein said crystal is characterized with space group p 2 21 21 and has unit cell parameters a=38.155±2Å; b=72.394±2Å; c=103.946±2Å; a=90°; b=90°; g=90°;
(b) determining the three-dimensional structure coordinates of BTK using the crystal in step (a);
(c) using the structure coordinates from step (b) to generate a three-dimensional structure of a molecular complex consisting essentially of a binding site of amino acid residues Leu408, Gly409, Thr410, Gly411, Va1416, Ala428, Lys430, Asn439, Met449, Leu452, Va1458, Ile472, Thr474, Glu475, Tyr476, Met477, Gly480, Cys481, Asn 484, Arg525, Leu528, Ser538, Asp539, and Phe540 according to Table 2, wherein the root mean square deviation of the backbone atoms is not greater than about 2.5 Å;
(d) employing said three-dimensional structure to design or select said candidate inhibitor;
(e) synthesizing said candidate inhibitor; and
(f) contacting said candidate inhibitor with BTK to determine the ability of said candidate inhibitor to bind to BTK.

### Methods for Determining a Candidate Inhibitors' Ability to Bind BTK

Aspects of the present disclosure also include the use of BTK binding assays to determine the ability of a candidate inhibitor to bind to BTK. In some aspects, a BTK binding assay is used to determine the level of BTK occupancy by a candidate inhibitor including but not limited to the compounds of the present disclosure. Also described herein are methods and kits for use in combination with the methods described herein to identify candidate inhibitors. In some aspects, the methods provided involve protein occupancy assays for one or more candidate inhibitors of BTK. Accordingly, described herein are protein occupancy assays for BTK inhibitors. Described herein in certain aspects is a protein occupancy assay that is an ELISA probe assay. In some aspects, the ELISA probe assay is plate based electrochemilummescent assay to determine the relative amount of a BTK that has not been bound by a candidate inhibitor. For example, in some aspects, the candidate inhibitor binds to the active site of the BTK and forms a disulfide bond with a cysteine residue. In some aspects, the assays involves binding an activity probe to free BTK that have not been bound by the candidate inhibitor. In some aspects, the activity probe comprises a BTK inhibitor attached to a detectable label (e.g., biotin) via a linker (e.g., a long chain linker). Labeling of samples with the probe allows for the detection of BTK not occupied by drug. In some aspects, the probe conjugated with the BTK is captured by a streptavidin coated plate. In some aspects, excess un-conjugated probe competes with probe labeled BTK for binding to streptavidin. Also described herein are methods for determining the efficacy of inhibitors of the BTK. Some aspects are methods for determining the efficacy of a protein modulator (e.g., inhibitor drug) on a target (e.g., target protein kinase). In some aspects, methods are provided for determining the efficacy of a candidate inhibitor on a target kinase (e.g., BTK). In some aspects, the method comprises: (a) contacting a sample comprising a BTK with a probe to form a probe-bound target kinase; (b) detecting the amount of the probe-bound target kinase in the sample; and (c) determining the efficacy of the candidate inhibitor based on the amount of probe-bound target kinase. In some aspects, the method further comprises contacting the sample with the candidate inhibitor prior to step (a) (e.g., combining the sample with the probe). In some aspects, detecting the amount of the probe-bound target kinase comprises administering a candidate inhibitor, reagent or buffer to detect the probe-bound kinase. In some aspects, the candidate inhibitor, reagent or buffer comprises horseradish peroxidase (HRP), detection antibody buffer, read buffer, wash buffer. In some aspects, detecting the presence or absence of the probe-bound target kinase comprises quantifying the amount of probe-bound target kinase. In some aspects, the quantifying step comprises fluorescence, immunofluorescence, chemiluminescence, or electrochemiluminescence. In some aspects, determining the efficacy of the candidate inhibitor comprises determining occupancy of the target kinase by the candidate inhibitor. In some aspects, the amount of probe-bound target kinase inversely correlates with the efficacy of the candidate inhibitor. For example, if a candidate inhibitor-treated sample (e.g., a sample that is contacted with the candidate inhibitor prior to contact with the probe such as a blood sample or tumor tissue) is contacted with the probe, then as the amount of probe-bound target kinases (e.g., unoccupied target kinases) detected increases, the efficacy of the candidate inhibitor decreases. In another example, if a drug-treated sample is contacted with the probe, then as the amount of probe-bound target kinase (e.g., unoccupied target kinases) detected decreases, the efficacy of the candidate inhibitor increases. In some aspects, the amount of probe-bound target kinases directly correlates with the efficacy of the candidate inhibitor. For example, if an untreated sample (e.g., a sample that is not contacted with the drug prior to contact with the probe) is contacted with the probe, then as the amount of probe-bound target kinase detected increases, the efficacy of the candidate inhibitor also increases. In another example, if an untreated sample (e.g., a sample that is not contacted with the candidate inhibitor prior to contact with the probe) is contacted with the probe, then as the amount of probe-bound target kinase detected decreases, the efficacy of the candidate inhibitor decreases. In some aspects, a candidate inhibitor is determined to be effective when the candidate inhibitor binds at least about 50% of the target kinases. Alternatively, a candidate inhibitor is determined to be effective when the drug binds at least about 60% of the target kinases. In some aspects, a candidate inhibitor is determined to be effective when the candidate inhibitor binds at least about 65%, 70%, 75%, 80%, 85%, 90%, 95%, 97%, or 99% of the targets. In some aspects, the assay is performed on a sample obtained from a subject (e,g, a mammal) that has been administered a candidate inhibitor. In some aspects, the sample is obtained about 1 hour, 2 hours, 4 hours, 6 hours, 8 hours, 10 hours, 12 hours, 14 hours, 16 hours, 18 hours, 20 hours, 22 hours, 24 hours, 30 hours, 36 hours, 42 hours, 48 hours, 3 days, 4, days, 5 days, 6 days, 1 week, 2 weeks or longer after administration of the candidate inhibitor. In some aspects, the probe comprises an agent and a label. In some instances, the agent is fused to the label. In other instances, the agent is attached to the label. In another instance, the agent is attached to the label by a linker. In some aspects, the agent and the candidate inhibitor are essentially the same. In some aspects, the probe comprises a label. In some aspects, the probe comprises a label and a linker. In some aspects, the agent and the candidate inhibitor are at least about 20% identical, at least about 30% identical, at least about 40% identical, at least about 50% identical, at least about 60% identical, at least about 70% identical, at least about 80% identical, at least about 90% identical, or at least about 95% identical. In other aspects, the agent and the candidate inhibitor are different. In some aspects, the agent and the candidate inhibitor are at least about 5% different, at least about 10% different, at least about 20% different, at least about 30% different, at least about 40% different, at least about 50% different, at least about 60%) different, at least about 70% different, at least about 80% different, at least about 90% different, or at least about 95% different.

Disclosed herein are protein occupancy assay kits comprising a linker, a label, an agent, or any combination thereof. In one aspect is a protein occupancy assay kit comprising a linker and a label, wherein the linker is capable of attaching the label to an agent and the agent is a protein modulator. In another aspect is a protein occupancy assay kit comprising an agent, a linker, and a label, wherein the linker is capable of attaching to the agent and the label, thereby attaching the agent to the label. In some aspects is a protein occupancy assay kit comprising a probe, wherein the probe comprises an agent attached to a label. In some aspects is a protein occupancy assay kit comprising a probe, wherein the probe comprises an agent attached to a linker. In some aspects is a protein occupancy assay kit comprising an agent and a solid support, wherein the agent is attached to the solid support. In another aspect is a protein occupancy assay kit comprising a label and a solid support, wherein the label is attached to the solid support. In another aspect is a protein occupancy assay kit comprising a probe and a solid support, wherein the probe comprises an agent, a linker, a label, or any combination thereof. In some aspects is a protein occupancy assay kit comprising a target (e.g., protein) and a solid support, wherein the target is attached to the solid support. In some aspects, any of the kits disclosed herein further comprise a label. In some aspects, any of the kits disclosed herein further comprise a linker. In some aspects, any of the kits disclosed herein further comprise an agent. In some aspects, any of the kits disclosed herein further comprise a plurality of linkers, wherein the linkers are capable of attaching to another linker, an agent, a label, or any combination thereof. In some aspects, any of the kits disclosed herein further comprise a probe. In some aspects the probe comprises an agent, a linker, a label, or any combination thereof. In some aspects, any of the kits disclosed herein further comprise a target (e.g., protein). Exemplary aspects of agents, linkers, labels, probes, solid supports, and targets are disclosed herein. Further disclosed herein are exemplary methods for attaching probes or targets to solid supports.

In some aspects, the methods, kits, and compositions disclosed herein comprise a probe. In some aspects, the probe comprises an agent and a label. In some aspects, the agent and label are attached. In other aspects, the probe comprises an agent and a linker. In some aspects, the agent and linker are attached. In another aspect, the probe comprises an agent, a linker, and a label. In some aspects, the agent, linker and/or label are attached to each other. In some aspects, the probe comprises a label. In another aspect, the probe comprises a label and a linker. In some aspects, the label and the linker are attached. In some aspects, attachment is by chemical methods, enzymatic methods, or crosslinking methods. In some aspects, the probe is attached to a solid support. Exemplary aspects of agents, linkers, labels, and solid supports are disclosed herein.

Any of the assays and systems disclosed herein can be useful in researching and validating a candidate inhibitor. Provided herein are methods for validating a candidate inhibitor comprising (a) contacting a sample comprising a target with a probe to form a probe-bound target; (b) detecting the presence or absence of the probe-bound target; and (c) determining occupancy of the target by a candidate inhibitor based on the presence or absence of the probe-bound target, thereby validating the candidate inhibitor.

Further provided herein are methods for determining occupancy of a target comprising: a) combining a sample comprising a target with a probe; b) detecting the presence or absence of a probe-bound target; and c) determining occupancy of the target by a candidate inhibitor based on the presence or absence of the probe-bound target.

In some aspects, the method further comprises capturing the target prior to step (a) contacting the sample with the probe. In some aspects, the target is captured by an antibody. In some aspects, the antibody is an anti-target antibody. In some aspects, the antibody is attached to a solid support. In some aspects, the solid support is a microplate. In some aspects, the microplate is a MSD microplate.

In yet other aspects, the method further comprises contacting the probe-bound target with a primary detection agent. In some aspects, the primary detection agent comprises an antibody, a bead, a dye, or a fluorophore. In some aspects, the primary detection agent comprises an antibody. In some aspects, the antibody is an anti-BTK antibody. In some aspects, the method further comprises contacting the detection agent with a secondary detection agent. In some aspects, the secondary detection agent comprises an antibody, a bead, a dye, or a fluorophore. In some aspects, the primary detection agent is labeled. In some aspects, the secondary detection agent is labeled. In some aspects, the label is an electrochemiluminescent tag. In some aspects, the electrochemiluminescent tag comprises Tris(bipyridine)ruthenium(II) dicfiloride. In some aspects, the electrochemiluminescent tag is Ruthenium (II) tris-bipyridine, N-hydroxysuccinimide. In some aspects, the label is a SULFO TAG.

In some aspects, detecting the presence or absence of the probe-bound target comprises contacting the sample with a solid support. In some aspects, the solid support comprises a bead. In some aspects, the bead is a streptavidin bead. In some aspects, the bead is a magnetic bead. In some aspects, the bead is a labeled bead. In some aspects, the bead is a labeled streptavidin bead. In some aspects, the bead is a labeled with an electrochemiluminescent tag. In some aspects, the electrochemiluminescent tag comprises Tris(bipyridine)ruthenium(II) dicfiloride. In some aspects, the electrochemiluminescent tag is Ruthenium (II) tris-bipyridine, N- hydroxysuccinimide. In some aspects, the bead is a SULFO TAG bead. In some aspects, the bead is a SULFO TAG streptavidin bead.

In some aspects, the bead interacts with the probe. In some aspects, the probe comprises a label. In some aspects, the label comprises biotin. In some aspects, the bead interacts with biotin. In some aspects, the bead forms a conjugate with the probe-bound target. In some aspects, the bead is conjugated to the probe.

In some aspects, detecting the presence or absence of the probe-bound target comprises detecting the probe-bound target or a portion thereof. In some aspects, detecting the presence or absence of the probe-bound target comprises detecting the bead or a portion thereof. In some aspects, detecting the presence or absence of the probe-bound target comprises detecting the labeled bead. In some aspects, detecting the presence or absence of the probe-bound target comprises detecting an electrochemiluminescent tag. In some aspects, the electrochemiluminescent tag comprises Tris(bipyridine)ruthenium(II) dichloride. In some aspects, the electrochemiluminescent tag is Ruthenium (II) tris- bipyridine, N-hydroxysuccinimide. In some aspects, detecting the presence or absence of the probe-bound target comprises detecting a SULFO TAG. In some aspects, the detecting step comprises luminescence. In some aspects, the detecting step comprises electrochemiluminescence.

In some aspects, the method further comprises purification of the probe-bound target. In some aspects, the probe-bound target is an unoccupied target. In some aspects, the probe-bound target is a drug-occupied target. In another aspect, purification of the probe-bound target comprises magnetic separation of probe-bound targets from non-probe-bound targets.

In some aspects, the sample is a pre -treated sample, wherein the pre-treated sample is contacted with a drug prior to contact with the probe. In some aspects, the sample is a non-treated sample, wherein the sample is not contacted with a candidate inhibitor prior to contact with the label.

In some aspects, the probe comprises an agent. In some aspects, the probe comprises an agent and a linker. In some aspects, the probe comprises a label. In some aspects, the probe comprises a label and a linker. In some aspects, the agent is a known BTK inhibitor. In some aspects, the agent is a compound of Formula (I). In some aspects, the BTK inhibitor is a reversible BTK inhibitor. In some aspects, the agent is a, the BTK inhibitor is an irreversible BTK inhibitor. In some aspects, the agent is a, the BTK inhibitor is a selective, covalent BTK inhibitor. In some aspects, the agent is a, the BTK inhibitor forms a covalent bond with a cysteine residue of a Bruton's tyrosine kinase (BTK). In some aspects, the cysteine residue is cysteine 481. In some aspects, the agent is a, the BTK inhibitor is a compound of Formula (I).

In some aspects, validating the drug comprises determining the efficacy of the candidate inhibitor on a target. In some aspects, determining occupancy of the target by the drug comprises quantifying the presence or absence of probe-bound targets. In some aspects, the candidate inhibitor is effective when the occupancy of the target is at least about 50%, 60%, 65%, 70%, 75%, 80%, 85%, 90%, 95%, 97%, or 99%.

Further disclosed herein is a method for determining efficacy of a test agent comprising: (a) combining a sample comprising a target with a probe; (b) detecting the presence or absence of a probe-bound target; and (c) determining efficacy of a test agent based on the presence or absence of the probe-bound target. Further disclosed herein is a method for identifying drug responders comprising: (a) combining a sample comprising a target with a probe; (b) detecting the presence or absence of a probe-bound target; and (c) identifying drug responders based on the presence or absence of the probe-bound target. Further disclosed herein is a method for identifying BTK inhibitors comprising: (a) combining a sample comprising a target with a probe; (b) detecting the presence or absence of a probe-bound target; and (c) identifying kinase modulators based on the presence or absence of the probe-bound target. Disclosed herein is a method for determining drug resistance comprising: (a) combining a sample comprising a target with a probe; (b) detecting the presence or absence of a probe-bound target; and (c) determining drug resistance based on the presence or absence of the probe-bound target.

In some aspects, the methods, assays, and systems disclosed herein comprise contacting sample comprising a target with a probe. Suitable samples for use in any of the methods, assays, and systems disclosed herein comprise, but are not limited to, a whole blood sample, peripheral blood sample, lymph sample, tissue sample, tumor biopsy sample, bone marrow sample, or other bodily fluid sample. In some aspects, the sample is a sample containing one or more cell types, or a lysate thereof, derived from a whole blood sample, peripheral blood sample, lymph sample, tissue sample, tumor biopsy sample, bone marrow sample, or other bodily fluid sample. Examples of bodily fluids include, but are not limited to, smears, sputum, biopsies, secretions, cerebrospinal fluid, bile, blood, lymph fluid, saliva, and urine. In some aspects, cells of the sample are isolated from other components of the sample prior to use in the methods provided. In some aspects, particular cell types of the sample are isolated from other cell types of the sample prior to use in the methods provided. For example, in some aspects, peripheral blood mononuclear cells (PBMCs, e.g., lymphocytes, monocytes and macrophages) of a blood sample are isolated from other cell types of the blood sample prior to use in the methods provided. For example, in some aspects, lymphocytes (e.g., B cells, T cells or NK cells) of the sample are isolated from other cell types of the sample prior to use in the methods provided. For example, in some aspects, B cells of the sample are isolated from other cell types of the sample prior to use in the methods provided. In some aspects, cells of the sample are lysed prior to use in the methods provided. For example, in some aspects, cancer cells are isolated from normal cells of the sample prior to use in the methods provided.

Any of the samples disclosed herein comprises complex populations of cells, which can be assayed as a population, or separated into sub-populations. Such cellular and acellular samples can be separated by centrifugation, elutriation, density gradient separation, apheresis, affinity selection, panning, FACS, filtration, centrifugation with Hypaque, etc. By using antibodies specific for markers identified with particular cell types, a relatively homogeneous population of cells can be obtained. Alternatively, a heterogeneous cell population can be used.

Once a sample is obtained, it can be used directly, frozen, or maintained in appropriate culture medium for short periods of time. Methods to isolate one or more cells for use according to the methods of this invention are performed according to standard techniques and protocols well-established in the art. In some aspects, the sample is obtained from a subject. Such subject can be a human or a domesticated animal such as a cow, chicken, pig, horse, rabbit, dog, cat, or goat. In some aspects, the cells used in the present invention are taken from a patient. Samples derived from an animal, e.g., human, can include, for example whole blood, sweat, tears, saliva, ear flow, sputum, lymph, bone marrow suspension, lymph, urine, saliva, semen, vaginal flow, cerebrospinal fluid, brain fluid, ascites, milk, secretions of the respiratory, intestinal or genitourinary tracts fluid, a lavage of a tissue or organ (e.g., lung) or tissue which has been removed from organs, such as breast, lung, intestine, skin, cervix, prostate, pancreas, heart, liver and stomach.

To obtain a blood sample, any technique known in the art can be used, e.g., a syringe or other vacuum suction device. A sample can be optionally pre -treated or processed prior to enrichment. Examples of pre-treatment steps include the addition of a reagent such as a stabilizer, a preservative, a fixant, a lysing reagent, a diluent, a drug, an anti-apoptotic reagent, an anti-coagulation reagent, an anti-thrombotic reagent, magnetic property regulating reagent, a buffering reagent, an osmolality regulating reagent, a pH regulating reagent, and/or a cross-linking reagent. For example, when a blood sample is obtained, a preservative such an anticoagulation agent and/or a stabilizer can be added to the sample prior to enrichment.

A sample, such as a blood sample, can be analyzed under any of the methods, assays and systems disclosed herein within 1 week, 6 days, 5 days, 4 days, 3 days, 2 days, 1 day, 12 hrs, 6 hrs, 3 hrs, 2 hrs, or 1 hr from the time the sample is obtained.

In some aspects, a sample can be combined with an enzyme or compound that selectively lyses one or more cells or components in the sample. For example, in a blood sample, platelets and/or enucleated red blood cells are selectively lysed to generate a sample enriched in nucleated cells. The cells of interest can subsequently be separated from the sample using methods known in the art.

When obtaining a sample from a subject (e.g., blood sample), the amount can vary depending upon subject size and the condition being screened. In some aspects, up to 50, 40, 30, 20, 10, 9, 8, 7, 6, 5, 4, 3, 2, or 1 mL of a sample is obtained. In some aspects, 1-50, 2-40, 3-30, or 4-20 mL of sample is obtained. In some aspects, more than 5, 10, 15, 20, 25, 30, 35, 40, 45, 50, 55, 60, 65, 70, 75, 80, 85, 90, 95 or 100 mL of a sample is obtained.

Aspects of the present disclosure include methods of monitoring the efficacy of a candidate inhibitor. In some aspects, a BTK occupancy of greater than about 75% is indicative that the candidate inhibitor is therapeutically effective. In some aspects, a BTK occupancy of greater than about 80% is indicative that the candidate inhibitor is therapeutically effective. In some aspects, a BTK occupancy of greater than about 90% is indicative that the candidate inhibitor is therapeutically effective. In some aspects, a BTK occupancy of greater than about 95% is indicative that the candidate inhibitor is therapeutically effective. In some aspects, a BTK occupancy of greater than about 99% is indicative that the candidate inhibitor is therapeutically effective. In some aspects, a BTK occupancy of greater than about 100% is indicative that the candidate inhibitor is therapeutically effective.

Aspects of the present disclosure include the use of a BTK lanthascreen binding assay to determine the BTK occupancy by a BTK inhibitor. In some aspects, a BTK lanthascreen binding assay monitors compound binding to unphosphorylated-BTK kinase domain (UP-BTK), by competing with a fluorescent labeled tracer. In some aspects, UP-BTK, consisting of the kinase domain of non-phosphorylated BTK protein (389-659aa), is produced in a Baculovirus/insect cell expression system. In some aspects, into a 384-well plate, 2 ng of GST-tagged human BTK (389-659aa) is incubated with a BTK inhibitor compound, 50 nM of Tracer 236 and 2 nM anti-GST antibody for 60 minutes using an optimized Lanthascreen™ assay. In some aspects, after 60 minutes, plates are read at 340 nM and 615/665 nM in a multifunctional plate reader such as an Infinite F500 (Tecan). In some aspects, data is analyzed using X1fit™ version 5.3 from ID Business Solutions (Guildford), Microsoft Excel add-in.

In some aspects, binding of a candidate inhibitor to BTK in the assay described herein may be indicative of the candidate inhibitors' function when used to treat a disease or condition in a patient in need thereof. In some aspects, a BTK occupancy of greater than about 75% is indicative that the candidate inhibitor is therapeutically effective. In some aspects, binding of a candidate inhibitor to BTK in the assay described herein may be predictive of a compounds ability to inhibit BTK and thereby treat a disease or condition. In some aspects, a BTK occupancy of greater than about 75% is indicative that the candidate inhibitor is effectively inhibiting BTK. In yet other aspects binding of a candidate inhibitor to BTK in the assay described herein may be predictive of the in vivo activity of a particular candidate inhibitor based on having similar BTK occupancy in the assay. In some aspects, a BTK occupancy of greater than about 75% is indicative that the candidate inhibitor is therapeutically effective in vivo.

### EXAMPLES

The following examples are for exemplary purposes only and are in no way meant to limit the invention.

### Example 1: Cloning and Generation of Recombinant Baculovirus

All cloning steps were performed using standard molecular biology protocols using molecular biology kits obtained from Qiagen; restriction enzymes were obtained from New England Biolabs, PfuUltra™ DNA polymerase was obtained from Stratagene. pFastBacl, the Bac-to-Bac-system and all insect cell media/components were obtained from Invitrogen.

The gene for the kinase domain of human BTK was amplified from a first-strand cDNA (MegaMan™ Human Transcriptome Library, Stratagene). The coding region for amino acids 389-659 of human BTK was fused to a DNA-sequence coding for GST followed by a TEV-protease cleavage site and cloned into vector pFastBacl using BamHI and XhoI restriction sites. After DNA sequencing to confirm integrity of the complete insert, the plasmid was used to generate recombinant bacmid-DNA using the Bac-to-Bac system. DNA and protein sequences are represented as SEQ ID NO: 1 (BTK DNA), SEQ ID NO: 2 (GST-portion of BTK protein), and SEQ ID NO: 3 (BTK kinase domain protein).

The bacmid-DNA was transfected into Sf9 cells to generate recombinant baculovirus (P1-virus) according to standard protocols. P2-viral stocks were amplified by infecting 15mL of 519 cells (0.5×106 cells/mL in a 75 cm2 dish) in Grace's medium supplemented with 10% FCS and 0.1% Pluronic with 500 μL of P1-virus and incubation at 26° C. for 1 week.

For generation of high titer virus stock (HTVS), 500 mL of SD cells (2×106 cells/mL in a glass spinner flask) in Grace's medium supplemented with 10% FCS and 0.1% Pluronic were infected with 4 mL of viral P2-stock and incubated at 26° C. for 6 days.

### Example 2: Expression of Recombinant Human BTK Kinase Domain

For expression of recombinant the human BTK kinase domain by a titerless infection protocol, 5 L of Sf9 (2×106 cells/mL in a 10 L cultibag, Sartorius Stedim) in Grace's medium supplemented with 10% FCS and 0.1% Pluronic were infected with 140 mL of HTVS (generated as described above) and grown for 64 hours on a BioWave 50 SPS (Wave Biotech) with aeration (0.1 L/min, 21 rocking motions per minute at a 10° angle) at 26° C. After expression, cells were harvested by centrifugation and stored frozen (−80° C.) until purification.

### Example 3: Purification of Recombinant Human BTK Kinase Domain

Buffers were prepared and the pH values adjusted at room temperature. For preparation of the PBS buffer, a 10× stock solution was diluted. All chromatography buffers were prepared and the pH adjusted at room temperature. Chromatography buffers were filtered (0.22 ∥m), degassed and cooled to 4° C. prior to use. Reducing agents were added immediately before use.
10×PBS
1.4 M NaCl
100 mM Na₂HPO₄
18 mM NaH₂PO₄
27 mM KCl

### Buffer A

1×PBS
5 mM B-ME
Final pH (HCl)=7.3 (at RT)

### Buffer B

20 mM Tris, pH=8.0
150 mM NaCl
10 mM reduced Glutathione (GSH)
2 mM DTT
Final pH (HCl)=8.0 (at RT)

### Buffer C

20 mM Tris/HCl, pH=8.0
150 mM NaCl
2 mM DTT

If not stated otherwise, all purification steps were performed at 4° C. on chromatography stations and columns obtained from GE Health Care (AKTA system). Between purification steps, the protein was kept on ice or in a cold room/fridge at 4° C. Recombinant TEV protease was prepared in house as a His-tagged protein (expressed in E. coli as soluble protein). All buffers were prepared and the pH adjusted at room temperature and then cooled to 4° C. Chromatography buffers were filtered (0.22 pm) and degassed prior to use. Reducing agents were added immediately before use. Before the protein was pooled, samples were analyzed on pre-cast SDS-gels (10%) obtained from Invitrogen. The isolated human BTK kinase domain tends to precipitate during purification which results in relatively low yield of purified protein.

Frozen cell pellets from a 5 L expression culture were thawed in 200 mL of buffer A (1×PBS, 5 mM β-ME) supplemented with four “Complete, EDTAfree Protease Inhibitor Cocktail Tablets” (Roche Applied Science) and disrupted using an Ultrathurax (Heidolph). Insoluble matter was pelleted by centrifugation at 51,000 g for 30 minutes. The initial capture step was performed in batch mode in that the cleared lysate was mixed with 10 mL GSH-Sepharose beads (GE Health Care) equilibrated in buffer A and incubated at 4° C. for 2 hours. The mixture was centrifuged for 10 minutes at 700 g to pellet the GSH-beads and the supernatant was discarded. Beads were washed twice with 40 mL buffer A and centrifuged as above. The washed beads were mixed with 5 mL buffer A and filled in an empty chromatography column (XK 16/20, GE Health Care). The column was packed by gravity flow, connected to an AKTA prime system and washed with buffer A at a flow rate of 2 mL/min. After UV-absorption at 280 nm reached a stable baseline, the buffer was changed to buffer B (20 mM Tris, pH=8.0, 150 mM NaCl, 10 mM reduced Glutathione (GSH), 2 mM DTT) and protein bound to the column was eluted at a flow rate of 2 mL/min.

Fractions containing GST-tagged human BTK kinase domain were pooled after SDS-Gel analysis and the protein concentration was determined to be 2.1 mg/mL by a standard Bradford assay.

The pooled protein was digested over night with 1.5 mg recombinant His-tagged TEV-protease, thereby dialyzing against 2 L of buffer A. Protein precipitated during dialysis was pelleted by centrifugation for 10 minutes at 4000 g and the supernatant of the centrifugation step was applied on the column used for the first affinity-step equilibrated in buffer A at a flow rate of 2 mL/min. The flow-through of the column was collected and after washing with buffer A, bound protein was eluted with 100% buffer B. This step was immediately repeated using the collected flow-through of the first run.

The pooled flow-through of the second run was concentrated to a volume of approximately 18 mL using a Millipore Amicon ultrafiltration device with a 10 kDa cutoff according to the manufacturer's instructions. The concentrated sample was buffer exchanged by size exclusion chromatography on a Superdex75 26/60 column equilibrated in buffer B 20 mM Tris/HCl, pH=8.0, 150 mM NaCl, 2 mM DTT) in two separate runs at a flow rate of 2 mL/min. In order to remove trace amounts of His-tagged TEV-protease, a 1 mL HisTrap-column was directly connected to the outlet of the Superdex-column. This procedure resulted in efficient removal of contaminating proteins, however, peak-broadening by the HisTrap-column had to be accepted.

Human BTK kinase domain eluted in a peak centered around a retention volume of approximately 190 mL. Pure protein was pooled after SDS-Gel analysis and concentrated as above to a final concentration of approximately 8-13 mg/mL as determined by a standard Bradford assay. The procedure described here yields roughly 2.5 mg of protein suitable for crystallization from a 5 L expression culture.

### Example 4: Crystallization of Recombinant Human BTK Kinase Domain

Human BTK kinase domain residues 389-659 in 20 mM Tris pH 8.0, 150 mM NaCl and 2 mM dithiothreitol was mixed with 2-fold excess of Compound I, incubated overnight at 4° C. and concentrated to 8 mg/ml. Crystals of BTK:Compound I complex were grown by hanging drop vapour diffusion method using NeXtal EasyXtal 15 well plates at 4° C. Crystals were obtained overnight with a 1:1 protein complex to well solution drop (25-33% PEG MME 5K, 100 mM MES pH 6.35-6.75, 200 mM Ammonium Sulfate) and a 0.2 ratio of microseed was added to the drop. The microseed stock was generated using BTK complexed with 5 mM Adenosine crystals that were grown in similar conditions; these crystals were harvested into a stabilization solution and vortexed to create the seed stock.

### Example 5: Structure Determination and Refinement

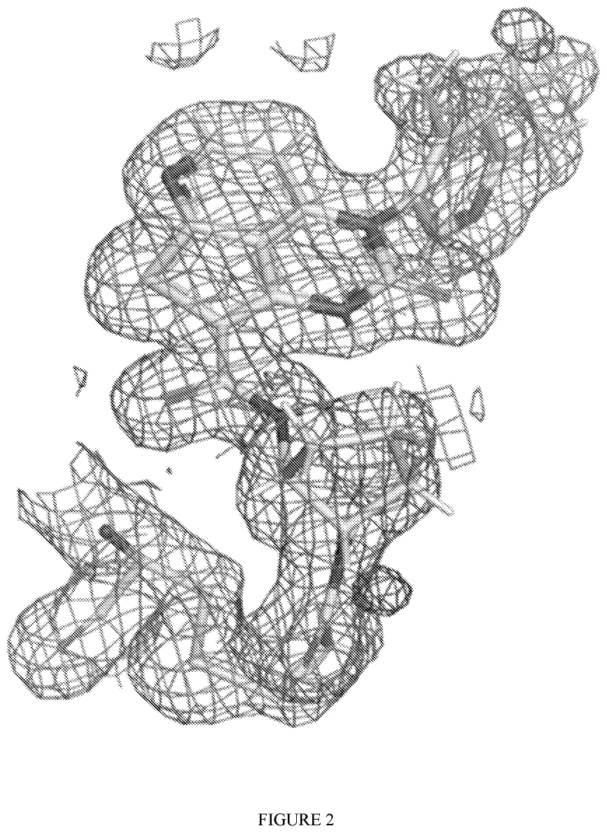

Crystals harvested were transferred briefly into a cryoprotectant solution composed of 80% well solution 20% Ethylene Glycol and flash frozen in liquid nitrogen. A dataset was collected using the IMCA17-ID beamline at Advanced Photon Source (Argonne National Laboratory). Data was processed with HKL2000 (Otwinowski and Minor, 1997). COOT was employed for model building. The phase information necessary to determine and analyze the structure was obtained by molecular replacement with a previously solved structure. Ligand restraints were generated in PHENIX. elbow and structure refined in PHENIX. refine (Liebschner et al., 2019). Coot (Emsley et al., 2010), MOE (Chemical Computing Group), and Pymol (Schrodinger) were used to analyze the structure. Crystals diffracted to 1.9 Å. Data collection and refinement statistics are presented in Table 1. Coordinates for the BTK-inhibitor complex are presented in Table 2. Unambiguous electron density was observed for the compound including a direct attachment to Cys 481 (FIG. 2).

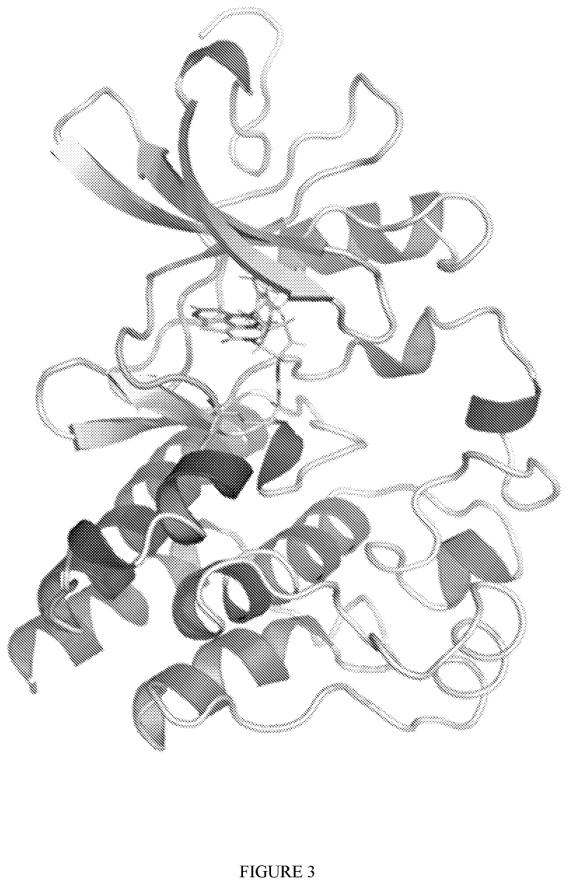

The structure of Human BTK in complex with Compound I adopts a bilobal architecture characteristic of other members of the eukaryotic protein kinase family. Situated in the cleft formed between the N-terminal and the C-terminal lobe the ligand is bound to the ATP binding site and neighboring regions of the active site (FIG. 3). There is one monomer in the asymmetric unit and the model comprises residues Gly389 to Glu568.

The amino acid residues forming the ligand binding site and the ligand were well defined in the electron density map. The interpreted X-ray diffraction data show a clear binding mode as well as orientation and conformation of the ligand bound to its binding site.

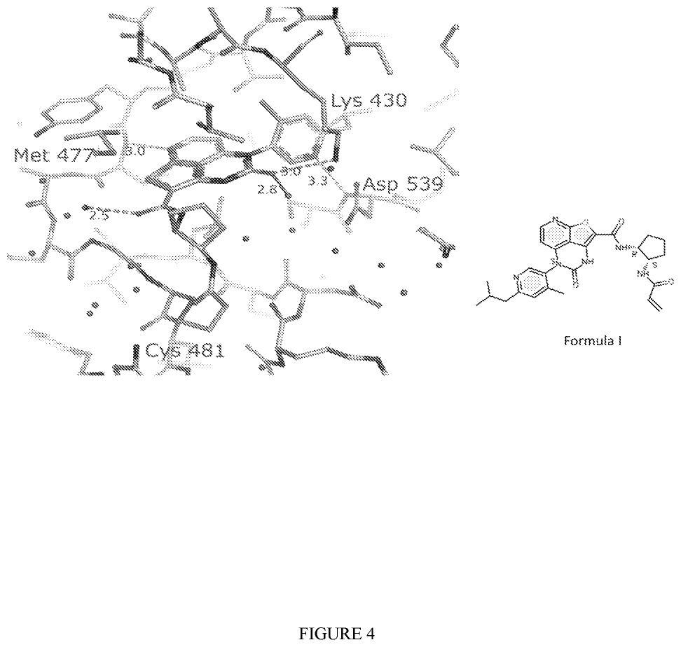

Results: A covalent bond between acrylamide moiety of Compound I and BTK Sulfur atom of Cys 481 was observed. Hydrogen bonds were observed between Compound I and residues Asp539, Lys430, and Met477. Solvent molecules were also observed in hydrogen bonds with Compound I (FIG. 4).

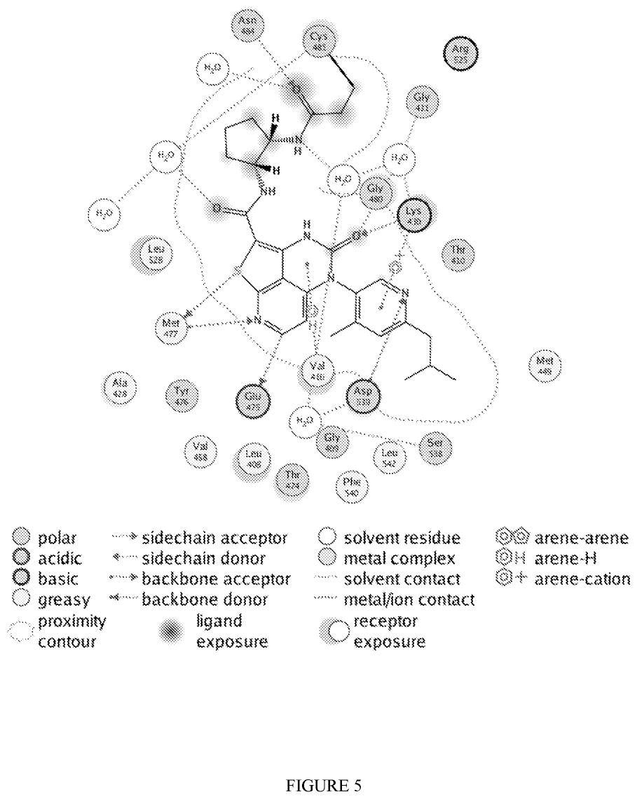

The following residues can be found in the vicinity of Compound I with a maximum distance of 4.0 Å; Leu408, Gly409, Thr410, Gly411, Va1416, Ala428, Lys430, Asn439, Met449, Leu452, Va1458, Ile472, Thr474, Glu475, Tyr476, Met477, Gly480, Cys481, Asn 484, Arg525, Leu528, Ser538, Asp539, and Phe540 (FIG. 5).

### REFERENCES

PHENIX: Macromolecular structure determination using X-rays, neutrons and electrons: recent developments in Phenix. D. Liebschner, P. V. Afonine, M. L. Baker, G. Bunkóczi, V. B. Chen, T. I. Croll, B. Hintze, L.-W. Hung, S. Jain, A. J. McCoy, N. W. Moriarty, R. D. Oeffner, B. K. Poon, M. G. Prisant, R. J. Read, J. S. Richardson, D. C. Richardson, M. D. Sammito, O. V. Sobolev, D. H. Stockwell, T. C. Terwilliger, A. G. Urzhumtsev, L. L. Videau, C. J. Williams, and P. D. Adams; Acta Cryst. (2019). D75, 861-877

COOT: Paul Emsley and Bernhard Lohkamp and William G. Scott and Kevin Cowtan, Features and Development of Coot, Acta Crystallographica Section D—Biological Crystallography,2010,Vol 66,486-501

HKL-2000: Z. Otwinowski and W. Minor, “Processing of X-ray Diffraction Data Collected in Oscillation Mode”, Methods in Enzymology, Volume 276: Macromolecular Crystallography, part A, p.30′7-326, 1997,C.W. Carter, Jr. & R. M. Sweet, Eds., Academic Press (New York).

IMCA-CAT Statement of Acknowledgment: Use of the IMCA-CAT beamline 17-ID (or 17-BM) at the Advanced Photon Source was supported by the companies of the Industrial Macromolecular Crystallography Association through a contract with Hauptman-Woodward Medical Research Institute.

This research used resources at the Industrial Macromolecular Crystallography Association Collaborative Access Team (IMCA-CAT) beamline 17-ID, supported by the companies of the Industrial Macromolecular Crystallography Association through a contract with Hauptman-Woodward Medical Research Institute.

Advanced Photon Source (APS) Statement of Acknowledgment: This research used resources of the Advanced Photon Source, a U.S. Department of Energy (DOE) Office of Science User Facility operated for the DOE Office of Science by Argonne National Laboratory under Contract No. DE-ACO2-06CH11357.

TABLE 1
Data collection and refinement statistics

| Wavelength | 1 | |
|---|---|---|
| Resolution range | 38.15-1.904 | (1.972-1.904) |
| Space group | P 2 21 21 | |
| Unit cell | 38.155 72.394 103.946 90 90 90 | |
| Total reflections | 98751 | |
| Unique reflections | 23063 | (2142) |
| Multiplicity | 4.3 | (4.1) |
| Completeness (%) | 99.04 | (94.78) |
| Mean I/sigma(I) | 186.3/12.3 | (37.9/10.7) |
| Wilson B-factor | 14.67 | |
| R-merge | 0.124 | (0.543) |
| R-meas | 0.141 | (0.618) |
| R-pim | 0.065 | (0.290) |
| Reflections used in refinement | 23063 | (2142) |
| Reflections used for R-free | 2000 | (186) |
| R-work | 0.1542 | (0.1854) |
| R-free | 0.1991 | (0.2778) |

| | |
|---|---|
| Number of non-hydrogen atoms | 2603 |
| macromolecules | 2295 |
| ligands | 47 |
| solvent | 261 |
| Protein residues | 271 |
| RMS(bonds) | 0.007 |
| RMS (angles) | 0.84 |
| Ramachandran favored (%) | 99.25 |
| Ramachandran allowed (%) | 0.75 |
| Ramachandran outliers (%) | 0 |
| Rotamer outliers (%) | 0 |
| Clashscore | 5.05 |
| Average B-factor | 18.77 |
| macromolecules | 17.73 |
| ligands | 20.07 |
| solvent | 27.72 |
| Number of TLS groups | 6 |

Table 2 lists the atomic structure coordinates in Protein Data Bank (PDB)-like format and header for human BTK in complex with Compound I, as derived by X-ray diffraction from crystals of the complex as obtained by ligand cocrystallization.

The following abbreviations are used in Table 2: Residue “LIG” represents Compound I. Structure figures were generated using PyMOL (The PyMOL Molecular Graphics System, Version 2.4.0 Schrodinger, LLC).

TABLE 2
Coordinates of BTK-Compound 1 Complex

| CRYST1 | 38.155 | | 72.394 | | 103.946 | 90.00 | 90.00 | 90.00 | P 2 21 21 | | | | |
|---|---|---|---|---|---|---|---|---|---|---|---|---|---|
| SCALE1 | 0.026209 | | | 0.000000 | | 0.000000 | | 0.00000 | | | | | |
| SCALE2 | 0.000000 | | | 0.013813 | | 0.000000 | | 0.00000 | | | | | |
| SCALE3 | 0.000000 | | | 0.000000 | | 0.009620 | | 0.00000 | | | | | |
| ATOM | 1 | N | GLY | A | 389 | −4.371 | −28.126 | −12.897 | 1.00 | 32.45 | | | N |
| ANISOU | 1 | N | GLY | A | 389 | 3958 | 4837 | 3534 | 731 | 766 | 70 | | N |
| ATOM | 2 | CA | GLY | A | 389 | −3.699 | −28.223 | −14.178 | 1.00 | 27.84 | | | C |
| ANISOU | 2 | CA | GLY | A | 389 | 3387 | 4326 | 2864 | 804 | 915 | 38 | | C |
| ATOM | 3 | C | GLY | A | 389 | −2.264 | −27.734 | −14.110 | 1.00 | 28.41 | | | C |
| ANISOU | 3 | C | GLY | A | 389 | 3237 | 4567 | 2992 | 836 | 1013 | 141 | | C |
| ATOM | 4 | O | GLY | A | 389 | −1.742 | −27.451 | −13.019 | 1.00 | 30.59 | | | O |
| ANISOU | 4 | O | GLY | A | 389 | 3339 | 4905 | 3379 | 815 | 937 | 215 | | O |
| ATOM | 5 | N | LEU | A | 390 | −1.623 | −27.647 | −15.273 | 1.00 | 25.19 | | | N |
| ANISOU | 5 | N | LEU | A | 390 | 2827 | 4239 | 2505 | 891 | 1183 | 138 | | N |
| ATOM | 6 | CA | LEU | A | 390 | −0.246 | −27.170 | −15.343 | 1.00 | 29.02 | | | C |
| ANISOU | 6 | CA | LEU | A | 390 | 3087 | 4856 | 3085 | 888 | 1284 | 227 | | C |
| ATOM | 7 | CB | LEU | A | 390 | 0.195 | −27.078 | −16.808 | 1.00 | 29.57 | | | C |
| ANISOU | 7 | CB | LEU | A | 390 | 3241 | 4932 | 3061 | 901 | 1441 | 207 | | C |
| ATOM | 8 | CG | LEU | A | 390 | 1.560 | −26.468 | −17.141 | 1.00 | 29.18 | | | C |
| ANISOU | 8 | CG | LEU | A | 390 | 2983 | 4990 | 3114 | 864 | 1579 | 292 | | C |
| ATOM | 9 | CD1 | LEU | A | 390 | 2.714 | −27.361 | −16.686 | 1.00 | 30.83 | | | C |
| ANISOU | 9 | CD1 | LEU | A | 390 | 3000 | 5249 | 3466 | 1006 | 1623 | 291 | | C |
| ATOM | 10 | CD2 | LEU | A | 390 | 1.659 | −26.166 | −18.634 | 1.00 | 30.82 | | | C |
| ANISOU | 10 | CD2 | LEU | A | 390 | 3336 | 5196 | 3180 | 858 | 1730 | 286 | | C |
| ATOM | 11 | C | LEU | A | 390 | −0.123 | −25.813 | −14.654 | 1.00 | 26.06 | | | C |
| ANISOU | 11 | C | LEU | A | 390 | 2542 | 4570 | 2788 | 711 | 1213 | 320 | | C |
| ATOM | 12 | O | LEU | A | 390 | −0.936 | −24.915 | −14.881 | 1.00 | 24.74 | | | O |
| ANISOU | 12 | O | LEU | A | 390 | 2490 | 4359 | 2551 | 569 | 1158 | 331 | | O |
| ATOM | 13 | N | GLY | A | 391 | 0.882 | −25.674 | −13.786 | 1.00 | 25.72 | | | N |
| ANISOU | 13 | N | GLY | A | 391 | 2239 | 4628 | 2907 | 711 | 1178 | 370 | | N |
| ATOM | 14 | CA | GLY | A | 391 | 1.134 | −24.422 | −13.107 | 1.00 | 25.04 | | | C |
| ANISOU | 14 | CA | GLY | A | 391 | 1975 | 4609 | 2928 | 526 | 1089 | 421 | | C |
| ATOM | 15 | C | GLY | A | 391 | 0.505 | −24.273 | −11.729 | 1.00 | 32.70 | | | C |
| ANISOU | 15 | C | GLY | A | 391 | 2960 | 5534 | 3930 | 466 | 847 | 394 | | C |
| ATOM | 16 | O | GLY | A | 391 | 0.841 | −23.313 | −11.020 | 1.00 | 31.96 | | | O |
| ANISOU | 16 | O | GLY | A | 391 | 2708 | 5499 | 3937 | 326 | 747 | 405 | | O |
| ATOM | 17 | N | TYR | A | 392 | −0.382 | −25.178 | −11.318 | 1.00 | 25.89 | | | N |
| ANISOU | 17 | N | TYR | A | 392 | 2283 | 4563 | 2991 | 564 | 761 | 354 | | N |
| ATOM | 18 | CA | TYR | A | 392 | −1.048 | −25.086 | −10.018 | 1.00 | 26.55 | | | C |
| ANISOU | 18 | CA | TYR | A | 392 | 2408 | 4598 | 3081 | 528 | 567 | 345 | | C |
| ATOM | 19 | CB | TYR | A | 392 | −2.559 | −24.865 | −10.184 | 1.00 | 18.80 | | | C |
| ANISOU | 19 | CB | TYR | A | 392 | 1673 | 3463 | 2009 | 437 | 517 | 309 | | C |
| ATOM | 20 | CG | TYR | A | 392 | −2.868 | −23.562 | −10.893 | 1.00 | 23.52 | | | C |
| ANISOU | 20 | CG | TYR | A | 392 | 2300 | 4055 | 2580 | 262 | 544 | 319 | | C |
| ATOM | 21 | CD1 | TYR | A | 392 | −3.006 | −22.374 | −10.180 | 1.00 | 23.23 | | | C |
| ANISOU | 21 | CD1 | TYR | A | 392 | 2207 | 4025 | 2595 | 109 | 438 | 330 | | C |
| ATOM | 22 | CE1 | TYR | A | 392 | −3.259 | −21.173 | −10.826 | 1.00 | 23.49 | | | C |
| ANISOU | 22 | CE1 | TYR | A | 392 | 2279 | 4025 | 2621 | −37 | 483 | 357 | | C |
| ATOM | 23 | CZ | TYR | A | 392 | −3.372 | −21.152 | −12.206 | 1.00 | 29.57 | | | C |
| ANISOU | 23 | CZ | TYR | A | 392 | 3151 | 4780 | 3304 | −16 | 630 | 386 | | C |
| ATOM | 24 | OH | TYR | A | 392 | −3.621 | −19.961 | −12.850 | 1.00 | 31.64 | | | O |
| ANISOU | 24 | OH | TYR | A | 392 | 3473 | 5003 | 3546 | −133 | 688 | 443 | | O |
| ATOM | 25 | CE2 | TYR | A | 392 | −3.235 | −22.319 | −12.943 | 1.00 | 25.53 | | | C |
| ANISOU | 25 | CE2 | TYR | A | 392 | 2701 | 4287 | 2714 | 138 | 724 | 359 | | C |
| ATOM | 26 | CD2 | TYR | A | 392 | −2.984 | −23.513 | −12.282 | 1.00 | 24.17 | | | C |
| ANISOU | 26 | CD2 | TYR | A | 392 | 2483 | 4124 | 2579 | 269 | 683 | 319 | | C |
| ATOM | 27 | C | TYR | A | 392 | −0.754 | −26.344 | −9.207 | 1.00 | 27.85 | | | C |
| ANISOU | 27 | C | TYR | A | 392 | 2554 | 4765 | 3264 | 734 | 527 | 359 | | C |
| ATOM | 28 | O | TYR | A | 392 | −1.061 | −27.457 | −9.645 | 1.00 | 28.78 | | | O |
| ANISOU | 28 | O | TYR | A | 392 | 2810 | 4779 | 3347 | 869 | 616 | 341 | | O |
| ATOM | 29 | N | GLY | A | 393 | −0.136 | −26.167 | −8.041 | 1.00 | 30.11 | | | N |
| ANISOU | 29 | N | GLY | A | 393 | 2673 | 5166 | 3602 | 768 | 392 | 386 | | N |
| ATOM | 30 | CA | GLY | A | 393 | 0.029 | −27.264 | −7.109 | 1.00 | 27.34 | | | C |
| ANISOU | 30 | CA | GLY | A | 393 | 2335 | 4814 | 3240 | 982 | 333 | 427 | | C |
| ATOM | 31 | C | GLY | A | 393 | −1.293 | −27.668 | −6.476 | 1.00 | 21.22 | | | C |
| ANISOU | 31 | C | GLY | A | 393 | 1810 | 3866 | 2385 | 987 | 282 | 435 | | C |
| ATOM | 32 | O | GLY | A | 393 | −2.332 | −27.038 | −6.664 | 1.00 | 19.53 | | | O |
| ANISOU | 32 | O | GLY | A | 393 | 1730 | 3555 | 2135 | 822 | 263 | 396 | | O |
| ATOM | 33 | N | SER | A | 394 | −1.254 | −28.766 | −5.711 | 1.00 | 25.76 | | | N |
| ANISOU | 33 | N | SER | A | 394 | 2445 | 4396 | 2945 | 1194 | 274 | 498 | | N |
| ATOM | 34 | CA | SER | A | 394 | −2.488 | −29.313 | −5.151 | 1.00 | 24.66 | | | C |
| ANISOU | 34 | CA | SER | A | 394 | 2542 | 4067 | 2760 | 1210 | 280 | 525 | | C |
| ATOM | 35 | CB | SER | A | 394 | −2.212 | −30.653 | −4.468 | 1.00 | 28.76 | | | C |
| ANISOU | 35 | CB | SER | A | 394 | 3123 | 4521 | 3283 | 1474 | 326 | 625 | | C |
| ATOM | 36 | OG | SER | A | 394 | −1.351 | −30.493 | −3.359 | 1.00 | 29.72 | | | O |
| ANISOU | 36 | OG | SER | A | 394 | 3103 | 4836 | 3353 | 1613 | 186 | 697 | | O |
| ATOM | 37 | C | SER | A | 394 | −3.150 | −28.361 | −4.163 | 1.00 | 24.13 | | | C |
| ANISOU | 37 | C | SER | A | 394 | 2510 | 4031 | 2625 | 1087 | 138 | 526 | | C |
| ATOM | 38 | O | SER | A | 394 | −4.357 | −28.473 | −3.920 | 1.00 | 28.46 | | | O |
| ANISOU | 38 | O | SER | A | 394 | 3239 | 4423 | 3151 | 1028 | 164 | 529 | | O |
| ATOM | 39 | N | TRP | A | 395 | −2.391 | −27.434 | −3.590 | 1.00 | 22.81 | | | N |
| ANISOU | 39 | N | TRP | A | 395 | 2169 | 4060 | 2438 | 1044 | −5 | 510 | | N |
| ATOM | 40 | CA | TRP | A | 395 | −2.921 | −26.466 | −2.642 | 1.00 | 19.85 | | | C |
| ANISOU | 40 | CA | TRP | A | 395 | 1830 | 3721 | 1990 | 935 | −145 | 483 | | C |
| ATOM | 41 | CB | TRP | A | 395 | −1.857 | −26.155 | −1.576 | 1.00 | 22.31 | | | C |
| ANISOU | 41 | CB | TRP | A | 395 | 1965 | 4256 | 2255 | 1033 | −325 | 482 | | C |
| ATOM | 42 | CG | TRP | A | 395 | −0.681 | −25.413 | −2.156 | 1.00 | 29.12 | | | C |
| ANISOU | 42 | CG | TRP | A | 395 | 2552 | 5282 | 3231 | 929 | −371 | 409 | | C |
| ATOM | 43 | CD1 | TRP | A | 395 | −0.492 | −24.059 | −2.163 | 1.00 | 22.82 | | | C |
| ANISOU | 43 | CD1 | TRP | A | 395 | 1636 | 4550 | 2483 | 710 | −463 | 310 | | C |
| ATOM | 44 | NE1 | TRP | A | 395 | 0.688 | −23.750 | −2.794 | 1.00 | 25.84 | | | N |
| ANISOU | 44 | NE1 | TRP | A | 395 | 1751 | 5061 | 3006 | 657 | −443 | 278 | | N |
| ATOM | 45 | CE2 | TRP | A | 395 | 1.285 | −24.910 | −3.219 | 1.00 | 29.87 | | | C |
| ANISOU | 45 | CE2 | TRP | A | 395 | 2203 | 5603 | 3544 | 859 | −339 | 353 | | C |
| ATOM | 46 | CD2 | TRP | A | 395 | 0.449 | −25.979 | −2.839 | 1.00 | 24.71 | | | C |
| ANISOU | 46 | CD2 | TRP | A | 395 | 1798 | 4818 | 2773 | 1034 | −296 | 433 | | C |
| ATOM | 47 | CE3 | TRP | A | 395 | 0.834 | −27.285 | −3.161 | 1.00 | 27.62 | | | C |
| ANISOU | 47 | CE3 | TRP | A | 395 | 2181 | 5156 | 3159 | 1261 | −178 | 513 | | C |
| ATOM | 48 | CZ3 | TRP | A | 395 | 2.034 | −27.480 | −3.833 | 1.00 | 30.79 | | | C |
| ANISOU | 48 | CZ3 | TRP | A | 395 | 2383 | 5640 | 3674 | 1292 | −109 | 499 | | C |
| ATOM | 49 | CH2 | TRP | A | 395 | 2.847 | −26.397 | −4.198 | 1.00 | 32.90 | | | C |
| ANISOU | 49 | CH2 | TRP | A | 395 | 2412 | 6031 | 4056 | 1106 | −144 | 422 | | C |
| ATOM | 50 | CZ2 | TRP | A | 395 | 2.489 | −25.106 | −3.905 | 1.00 | 33.02 | | | C |
| ANISOU | 50 | CZ2 | TRP | A | 395 | 2393 | 6076 | 4075 | 882 | −254 | 350 | | C |
| ATOM | 51 | C | TRP | A | 395 | −3.353 | −25.174 | −3.313 | 1.00 | 21.64 | | | C |
| ANISOU | 51 | C | TRP | A | 395 | 2043 | 3923 | 2255 | 680 | −152 | 396 | | C |
| ATOM | 52 | O | TRP | A | 395 | −3.790 | −24.251 | −2.614 | 1.00 | 19.86 | | | O |
| ANISOU | 52 | O | TRP | A | 395 | 1849 | 3713 | 1985 | 577 | −257 | 356 | | O |
| ATOM | 53 | N | GLU | A | 396 | −3.246 | −25.092 | −4.640 | 1.00 | 18.05 | | | N |
| ANISOU | 53 | N | GLU | A | 396 | 1561 | 3428 | 1871 | 597 | −34 | 372 | | N |
| ATOM | 54 | CA | GLU | A | 396 | −3.425 | −23.848 | −5.379 | 1.00 | 23.81 | | | C |
| ANISOU | 54 | CA | GLU | A | 396 | 2262 | 4149 | 2635 | 385 | −23 | 319 | | C |
| ATOM | 55 | CB | GLU | A | 396 | −2.202 | −23.541 | −6.245 | 1.00 | 27.13 | | | C |
| ANISOU | 55 | CB | GLU | A | 396 | 2481 | 4684 | 3143 | 349 | 49 | 315 | | C |
| ATOM | 56 | CG | GLU | A | 396 | −1.038 | −23.005 | −5.442 | 1.00 | 30.43 | | | C |
| ANISOU | 56 | CG | GLU | A | 396 | 2653 | 5279 | 3631 | 334 | −74 | 291 | | C |
| ATOM | 57 | CD | GLU | A | 396 | 0.143 | −22.658 | −6.308 | 1.00 | 37.54 | | | C |
| ANISOU | 57 | CD | GLU | A | 396 | 3320 | 6284 | 4658 | 277 | 27 | 292 | | C |
| ATOM | 58 | OE1 | GLU | A | 396 | 0.543 | −23.502 | −7.143 | 1.00 | 32.56 | | | O |
| ANISOU | 58 | OE1 | GLU | A | 396 | 2673 | 5661 | 4037 | 398 | 176 | 334 | | O |
| ATOM | 59 | OE2 | GLU | A | 396 | 0.659 | −21.533 | −6.153 | 1.00 | 38.01 | | | O |
| ANISOU | 59 | OE2 | GLU | A | 396 | 3219 | 6406 | 4819 | 107 | −29 | 246 | | O |
| ATOM | 60 | C | GLU | A | 396 | −4.665 | −23.909 | −6.256 | 1.00 | 22.03 | | | C |
| ANISOU | 60 | C | GLU | A | 396 | 2231 | 3758 | 2382 | 317 | 67 | 307 | | C |
| ATOM | 61 | O | GLU | A | 396 | −4.819 | −24.831 | −7.064 | 1.00 | 21.43 | | | O |
| ANISOU | 61 | O | GLU | A | 396 | 2225 | 3614 | 2302 | 396 | 174 | 308 | | O |
| ATOM | 62 | N | ILE | A | 397 | −5.524 | −22.909 | −6.117 | 1.00 | 16.44 | | | N |
| ANISOU | 62 | N | ILE | A | 397 | 1600 | 2990 | 1658 | 177 | 15 | 280 | | N |
| ATOM | 63 | CA | ILE | A | 397 | −6.723 | −22.793 | −6.937 | 1.00 | 13.45 | | | C |
| ANISOU | 63 | CA | ILE | A | 397 | 1373 | 2481 | 1254 | 112 | 67 | 262 | | C |
| ATOM | 64 | CB | ILE | A | 397 | −7.933 | −22.377 | −6.075 | 1.00 | 18.66 | | | C |
| ANISOU | 64 | CB | ILE | A | 397 | 2142 | 3053 | 1893 | 65 | −5 | 250 | | C |
| ATOM | 65 | CG1 | ILE | A | 397 | −8.477 | −23.610 | −5.329 | 1.00 | 18.40 | | | C |
| ANISOU | 65 | CG1 | ILE | A | 397 | 2191 | 2948 | 1852 | 190 | 15 | 275 | | C |
| ATOM | 66 | CD1 | ILE | A | 397 | −9.572 | −23.332 | −4.390 | 1.00 | 32.00 | | | C |
| ANISOU | 66 | CD1 | ILE | A | 397 | 4005 | 4596 | 3557 | 168 | −18 | 281 | | C |
| ATOM | 67 | CG2 | ILE | A | 397 | −8.995 | −21.678 | −6.918 | 1.00 | 15.22 | | | C |
| ANISOU | 67 | CG2 | ILE | A | 397 | 1800 | 2530 | 1452 | −36 | 9 | 226 | | C |
| ATOM | 68 | C | ILE | A | 397 | −6.455 | −21.796 | −8.053 | 1.00 | 13.64 | | | C |
| ANISOU | 68 | C | ILE | A | 397 | 1366 | 2530 | 1288 | −4 | 118 | 260 | | C |
| ATOM | 69 | O | ILE | A | 397 | −5.795 | −20.769 | −7.847 | 1.00 | 17.36 | | | O |
| ANISOU | 69 | O | ILE | A | 397 | 1729 | 3061 | 1806 | −102 | 92 | 267 | | O |
| ATOM | 70 | N | ASP | A | 398 | −6.948 | −22.107 | −9.245 | 1.00 | 13.46 | | | N |
| ANISOU | 70 | N | ASP | A | 398 | 1442 | 2455 | 1218 | 12 | 196 | 250 | | N |
| ATOM | 71 | CA | ASP | A | 398 | −6.868 | −21.172 | −10.361 | 1.00 | 13.84 | | | C |
| ANISOU | 71 | CA | ASP | A | 398 | 1510 | 2516 | 1234 | −69 | 261 | 274 | | C |
| ATOM | 72 | CB | ASP | A | 398 | −7.035 | −21.914 | −11.690 | 1.00 | 17.18 | | | C |
| ANISOU | 72 | CB | ASP | A | 398 | 2028 | 2933 | 1565 | 16 | 353 | 250 | | C |
| ATOM | 73 | CG | ASP | A | 398 | −6.839 | −21.011 | −12.907 | 1.00 | 24.12 | | | C |
| ANISOU | 73 | CG | ASP | A | 398 | 2948 | 3844 | 2370 | −28 | 446 | 302 | | C |
| ATOM | 74 | OD1 | ASP | A | 398 | −6.788 | −19.763 | −12.774 | 1.00 | 16.57 | | | O |
| ANISOU | 74 | OD1 | ASP | A | 398 | 1968 | 2876 | 1453 | −138 | 443 | 364 | | O |
| ATOM | 75 | OD2 | ASP | A | 398 | −6.747 | −21.569 | −14.015 | 1.00 | 26.52 | | | O |
| ANISOU | 75 | OD2 | ASP | A | 398 | 3330 | 4178 | 2570 | 60 | 534 | 282 | | O |
| ATOM | 76 | C | ASP | A | 398 | −7.944 | −20.108 | −10.180 | 1.00 | 12.93 | | | C |
| ANISOU | 76 | C | ASP | A | 398 | 1486 | 2318 | 1110 | −167 | 188 | 277 | | C |
| ATOM | 77 | O | ASP | A | 398 | −9.137 | −20.429 | −10.228 | 1.00 | 14.15 | | | O |
| ANISOU | 77 | O | ASP | A | 398 | 1751 | 2397 | 1228 | −142 | 138 | 240 | | O |
| ATOM | 78 | N | PRO | A | 399 | −7.581 | −18.845 | −9.941 | 1.00 | 16.51 | | | N |
| ANISOU | 78 | N | PRO | A | 399 | 1885 | 2773 | 1616 | −280 | 181 | 311 | | N |
| ATOM | 79 | CA | PRO | A | 399 | −8.623 | −17.834 | −9.722 | 1.00 | 12.64 | | | C |
| ANISOU | 79 | CA | PRO | A | 399 | 1493 | 2185 | 1125 | −351 | 121 | 313 | | C |
| ATOM | 80 | CB | PRO | A | 399 | −7.822 | −16.545 | −9.492 | 1.00 | 15.43 | | | C |
| ANISOU | 80 | CB | PRO | A | 399 | 1761 | 2529 | 1571 | −481 | 145 | 342 | | C |
| ATOM | 81 | CG | PRO | A | 399 | −6.539 | −16.791 | −10.206 | 1.00 | 20.92 | | | C |
| ANISOU | 81 | CG | PRO | A | 399 | 2337 | 3317 | 2296 | −483 | 263 | 383 | | C |
| ATOM | 82 | CD | PRO | A | 399 | −6.240 | −18.239 | −9.930 | 1.00 | 19.35 | | | C |
| ANISOU | 82 | CD | PRO | A | 399 | 2086 | 3205 | 2062 | −355 | 241 | 342 | | C |
| ATOM | 83 | C | PRO | A | 399 | −9.570 | −17.700 | −10.900 | 1.00 | 17.15 | | | C |
| ANISOU | 83 | C | PRO | A | 399 | 2203 | 2710 | 1604 | −315 | 147 | 338 | | C |
| ATOM | 84 | O | PRO | A | 399 | −10.713 | −17.273 | −10.712 | 1.00 | 16.72 | | | O |
| ANISOU | 84 | O | PRO | A | 399 | 2231 | 2581 | 1540 | −319 | 78 | 326 | | O |
| ATOM | 85 | N | LYS | A | 400 | −9.140 | −18.070 | −12.104 | 1.00 | 13.37 | | | N |
| ANISOU | 85 | N | LYS | A | 400 | 1750 | 2286 | 1045 | −260 | 241 | 366 | | N |
| ATOM | 86 | CA | LYS | A | 400 | −10.030 | −17.994 | −13.257 | 1.00 | 15.08 | | | C |
| ANISOU | 86 | CA | LYS | A | 400 | 2110 | 2488 | 1133 | −198 | 239 | 376 | | C |
| ATOM | 87 | CB | LYS | A | 400 | −9.255 | −18.271 | −14.551 | 1.00 | 15.76 | | | C |
| ANISOU | 87 | CB | LYS | A | 400 | 2229 | 2655 | 1103 | −131 | 373 | 416 | | C |
| ATOM | 88 | CG | LYS | A | 400 | −8.191 | −17.227 | −14.845 | 1.00 | 16.95 | | | C |
| ANISOU | 88 | CG | LYS | A | 400 | 2324 | 2819 | 1298 | −207 | 522 | 540 | | C |
| ATOM | 89 | CD | LYS | A | 400 | −7.400 | −17.537 | −16.093 | 1.00 | 23.88 | | | C |
| ANISOU | 89 | CD | LYS | A | 400 | 3234 | 3784 | 2056 | −126 | 694 | 594 | | C |
| ATOM | 90 | CE | LYS | A | 400 | −6.179 | −16.625 | −16.146 | 1.00 | 34.38 | | | C |
| ANISOU | 90 | CE | LYS | A | 400 | 4443 | 5116 | 3503 | −230 | 871 | 715 | | C |
| ATOM | 91 | NZ | LYS | A | 400 | −5.783 | −16.275 | −17.523 | 1.00 | 43.76 | | | N |
| ANISOU | 91 | NZ | LYS | A | 400 | 5741 | 6292 | 4595 | −181 | 1021 | 778 | | N |
| ATOM | 92 | C | LYS | A | 400 | −11.207 | −18.945 | −13.120 | 1.00 | 17.36 | | | C |
| ANISOU | 92 | C | LYS | A | 400 | 2452 | 2746 | 1397 | −135 | 127 | 272 | | C |
| ATOM | 93 | O | LYS | A | 400 | −12.196 | −18.797 | −13.847 | 1.00 | 13.70 | | | O |
| ANISOU | 93 | O | LYS | A | 400 | 2084 | 2272 | 850 | −92 | 66 | 252 | | O |
| ATOM | 94 | N | ASP | A | 401 | −11.125 | −19.909 | −12.203 | 1.00 | 12.09 | | | N |
| ANISOU | 94 | N | ASP | A | 401 | 1719 | 2063 | 810 | −124 | 100 | 211 | | N |
| ATOM | 95 | CA | ASP | A | 401 | −12.231 | −20.819 | −11.952 | 1.00 | 13.17 | | | C |
| ANISOU | 95 | CA | ASP | A | 401 | 1890 | 2139 | 976 | −90 | 25 | 120 | | C |
| ATOM | 96 | CB | ASP | A | 401 | −11.715 | −22.133 | −11.339 | 1.00 | 13.99 | | | C |
| ANISOU | 96 | CB | ASP | A | 401 | 1946 | 2227 | 1143 | −42 | 65 | 81 | | C |
| ATOM | 97 | CG | ASP | A | 401 | −11.015 | −23.028 | −12.358 | 1.00 | 21.28 | | | C |
| ANISOU | 97 | CG | ASP | A | 401 | 2894 | 3193 | 1998 | 39 | 142 | 37 | | C |
| ATOM | 98 | OD1 | ASP | A | 401 | −11.162 | −22.787 | −13.574 | 1.00 | 16.48 | | | O |
| ANISOU | 98 | OD1 | ASP | A | 401 | 2360 | 2628 | 1275 | 63 | 149 | 12 | | O |
| ATOM | 99 | OD2 | ASP | A | 401 | −10.329 | −23.983 | −11.938 | 1.00 | 19.56 | | | O |
| ANISOU | 99 | OD2 | ASP | A | 401 | 2634 | 2967 | 1831 | 99 | 198 | 28 | | O |
| ATOM | 100 | C | ASP | A | 401 | −13.283 | −20.223 | −11.023 | 1.00 | 12.56 | | | C |
| ANISOU | 100 | C | ASP | A | 401 | 1809 | 1990 | 975 | −139 | −53 | 124 | | C |
| ATOM | 101 | O | ASP | A | 401 | −14.244 | −20.921 | −10.693 | 1.00 | 12.86 | | | O |
| ANISOU | 101 | O | ASP | A | 401 | 1848 | 1967 | 1071 | −127 | −94 | 60 | | O |
| ATOM | 102 | N | LEU | A | 402 | −13.114 | −18.982 | −10.562 | 1.00 | 10.98 | | | N |
| ANISOU | 102 | N | LEU | A | 402 | 1598 | 1782 | 791 | −197 | −57 | 192 | | N |
| ATOM | 103 | CA | LEU | A | 402 | −14.025 | −18.381 | −9.591 | 1.00 | 9.88 | | | C |
| ANISOU | 103 | CA | LEU | A | 402 | 1463 | 1573 | 718 | −228 | −110 | 190 | | C |
| ATOM | 104 | CB | LEU | A | 402 | −13.235 | −17.599 | −8.536 | 1.00 | 9.82 | | | C |
| ANISOU | 104 | CB | LEU | A | 402 | 1416 | 1564 | 750 | −287 | −98 | 221 | | C |
| ATOM | 105 | CG | LEU | A | 402 | −12.249 | −18.441 | −7.750 | 1.00 | 9.85 | | | C |
| ANISOU | 105 | CG | LEU | A | 402 | 1347 | 1626 | 768 | −268 | −81 | 207 | | C |
| ATOM | 106 | CD1 | LEU | A | 402 | −11.376 | −17.527 | −6.882 | 1.00 | 10.30 | | | C |
| ANISOU | 106 | CD1 | LEU | A | 402 | 1349 | 1709 | 854 | −334 | −104 | 210 | | C |
| ATOM | 107 | CD2 | LEU | A | 402 | −13.003 | −19.450 | −6.888 | 1.00 | 9.86 | | | C |
| ANISOU | 107 | CD2 | LEU | A | 402 | 1366 | 1588 | 794 | −206 | −90 | 180 | | C |
| ATOM | 108 | C | LEU | A | 402 | −15.030 | −17.450 | −10.271 | 1.00 | 12.37 | | | C |
| ANISOU | 108 | C | LEU | A | 402 | 1839 | 1855 | 1007 | −219 | −157 | 209 | | C |
| ATOM | 109 | O | LEU | A | 402 | −14.683 | −16.704 | −11.187 | 1.00 | 10.89 | | | O |
| ANISOU | 109 | O | LEU | A | 402 | 1701 | 1688 | 749 | −215 | −133 | 270 | | O |
| ATOM | 110 | N | THR | A | 403 | −16.278 | −17.500 | −9.816 | 1.00 | 12.87 | | | N |
| ANISOU | 110 | N | THR | A | 403 | 1895 | 1866 | 1131 | −201 | −211 | 171 | | N |
| ATOM | 111 | CA | THR | A | 403 | −17.317 | −16.552 | −10.208 | 1.00 | 11.44 | | | C |
| ANISOU | 111 | CA | THR | A | 403 | 1746 | 1652 | 947 | −170 | −267 | 193 | | C |
| ATOM | 112 | CB | THR | A | 403 | −18.523 | −17.273 | −10.829 | 1.00 | 20.40 | | | C |
| ANISOU | 112 | CB | THR | A | 403 | 2847 | 2806 | 2098 | −116 | −352 | 114 | | C |
| ATOM | 113 | OG1 | THR | A | 403 | −18.121 | −17.981 | −12.009 | 1.00 | 19.53 | | | O |
| ANISOU | 113 | OG1 | THR | A | 403 | 2767 | 2770 | 1882 | −84 | −376 | 73 | | O |
| ATOM | 114 | CG2 | THR | A | 403 | −19.655 | −16.285 | −11.178 | 1.00 | 14.14 | | | C |
| ANISOU | 114 | CG2 | THR | A | 403 | 2062 | 1999 | 1311 | −55 | −429 | 141 | | C |
| ATOM | 115 | C | THR | A | 403 | −17.739 | −15.819 | −8.943 | 1.00 | 10.17 | | | C |
| ANISOU | 115 | C | THR | A | 403 | 1577 | 1414 | 873 | −193 | −251 | 205 | | C |
| ATOM | 116 | O | THR | A | 403 | −18.223 | −16.449 | −7.999 | 1.00 | 13.14 | | | O |
| ANISOU | 116 | O | THR | A | 403 | 1907 | 1767 | 1320 | −193 | −237 | 162 | | O |
| ATOM | 117 | N | PHE | A | 404 | −17.536 | −14.512 | −8.903 | 1.00 | 11.70 | | | N |
| ANISOU | 117 | N | PHE | A | 404 | 1827 | 1557 | 1062 | −209 | −234 | 264 | | N |
| ATOM | 118 | CA | PHE | A | 404 | −17.874 | −13.728 | −7.717 | 1.00 | 11.49 | | | C |
| ANISOU | 118 | CA | PHE | A | 404 | 1815 | 1447 | 1103 | −224 | −216 | 253 | | C |
| ATOM | 119 | CB | PHE | A | 404 | −16.950 | −12.513 | −7.592 | 1.00 | 14.83 | | | C |
| ANISOU | 119 | CB | PHE | A | 404 | 2297 | 1806 | 1531 | −293 | −176 | 291 | | C |
| ATOM | 120 | CG | PHE | A | 404 | −15.515 | −12.876 | −7.314 | 1.00 | 18.87 | | | C |
| ANISOU | 120 | CG | PHE | A | 404 | 2763 | 2375 | 2031 | −378 | −151 | 272 | | C |
| ATOM | 121 | CD2 | PHE | A | 404 | −14.605 | −13.008 | −8.344 | 1.00 | 25.13 | | | C |
| ANISOU | 121 | CD2 | PHE | A | 404 | 3541 | 3220 | 2786 | −408 | −113 | 326 | | C |
| ATOM | 122 | CE2 | PHE | A | 404 | −13.287 | −13.352 | −8.090 | 1.00 | 27.49 | | | C |
| ANISOU | 122 | CE2 | PHE | A | 404 | 3763 | 3584 | 3098 | −476 | −86 | 308 | | C |
| ATOM | 123 | CZ | PHE | A | 404 | −12.875 | −13.580 | −6.792 | 1.00 | 30.67 | | | C |
| ANISOU | 123 | CZ | PHE | A | 404 | 4110 | 4011 | 3530 | −506 | −127 | 232 | | C |
| ATOM | 124 | CE1 | PHE | A | 404 | −13.778 | −13.456 | −5.756 | 1.00 | 26.97 | | | C |
| ANISOU | 124 | CE1 | PHE | A | 404 | 3687 | 3494 | 3065 | −467 | −168 | 180 | | C |
| ATOM | 125 | CD1 | PHE | A | 404 | −15.087 | −13.106 | −6.021 | 1.00 | 23.07 | | | C |
| ANISOU | 125 | CD1 | PHE | A | 404 | 3262 | 2925 | 2579 | −409 | −165 | 202 | | C |
| ATOM | 126 | C | PHE | A | 404 | −19.330 | −13.288 | −7.809 | 1.00 | 20.16 | | | C |
| ANISOU | 126 | C | PHE | A | 404 | 2912 | 2498 | 2249 | −143 | −250 | 255 | | C |
| ATOM | 127 | O | PHE | A | 404 | −19.747 | −12.724 | −8.822 | 1.00 | 17.77 | | | O |
| ANISOU | 127 | O | PHE | A | 404 | 2644 | 2192 | 1915 | −84 | −289 | 307 | | O |
| ATOM | 128 | N | LEU | A | 405 | −20.097 | −13.540 | −6.748 | 1.00 | 14.31 | | | N |
| ANISOU | 128 | N | LEU | A | 405 | 2129 | 1727 | 1579 | −122 | −228 | 209 | | N |
| ATOM | 129 | CA | LEU | A | 405 | −21.532 | −13.302 | −6.769 | 1.00 | 14.05 | | | C |
| ANISOU | 129 | CA | LEU | A | 405 | 2048 | 1666 | 1624 | −39 | −248 | 202 | | C |
| ATOM | 130 | CB | LEU | A | 405 | −22.283 | −14.629 | −6.547 | 1.00 | 10.88 | | | C |
| ANISOU | 130 | CB | LEU | A | 405 | 1528 | 1306 | 1300 | −38 | −243 | 145 | | C |
| ATOM | 131 | CG | LEU | A | 405 | −22.241 | −15.649 | −7.702 | 1.00 | 21.29 | | | C |
| ANISOU | 131 | CG | LEU | A | 405 | 2790 | 2699 | 2599 | −53 | −321 | 108 | | C |
| ATOM | 132 | CD1 | LEU | A | 405 | −22.918 | −16.955 | −7.326 | 1.00 | 21.09 | | | C |
| ANISOU | 132 | CD1 | LEU | A | 405 | 2650 | 2667 | 2695 | −80 | −291 | 39 | | C |
| ATOM | 133 | CD2 | LEU | A | 405 | −22.870 | −15.090 | −8.961 | 1.00 | 18.23 | | | C |
| ANISOU | 133 | CD2 | LEU | A | 405 | 2393 | 2356 | 2177 | 22 | −438 | 117 | | C |
| ATOM | 134 | C | LEU | A | 405 | −22.004 | −12.269 | −5.752 | 1.00 | 17.39 | | | C |
| ANISOU | 134 | C | LEU | A | 405 | 2517 | 1994 | 2096 | −1 | −189 | 202 | | C |
| ATOM | 135 | O | LEU | A | 405 | −22.918 | −11.509 | −6.054 | 1.00 | 18.31 | | | O |
| ANISOU | 135 | O | LEU | A | 405 | 2629 | 2067 | 2262 | 84 | −207 | 228 | | O |
| ATOM | 136 | N | LYS | A | 406 | −21.423 | −12.213 | −4.552 | 1.00 | 12.32 | | | N |
| ANISOU | 136 | N | LYS | A | 406 | 1926 | 1324 | 1431 | −43 | −125 | 166 | | N |
| ATOM | 137 | CA | LYS | A | 406 | −21.906 | −11.283 | −3.536 | 1.00 | 14.08 | | | C |
| ANISOU | 137 | CA | LYS | A | 406 | 2211 | 1457 | 1681 | 6 | −64 | 138 | | C |
| ATOM | 138 | CB | LYS | A | 406 | −23.061 | −11.895 | −2.734 | 1.00 | 18.34 | | | C |
| ANISOU | 138 | CB | LYS | A | 406 | 2675 | 2008 | 2284 | 79 | 13 | 119 | | C |
| ATOM | 139 | CG | LYS | A | 406 | −23.658 | −10.968 | −1.693 | 1.00 | 24.26 | | | C |
| ANISOU | 139 | CG | LYS | A | 406 | 3494 | 2673 | 3050 | 159 | 100 | 86 | | C |
| ATOM | 140 | CD | LYS | A | 406 | −24.962 | −11.524 | −1.111 | 1.00 | 33.29 | | | C |
| ANISOU | 140 | CD | LYS | A | 406 | 4534 | 3829 | 4286 | 243 | 207 | 92 | | C |
| ATOM | 141 | CE | LYS | A | 406 | −24.760 | −12.877 | −0.437 | 1.00 | 39.62 | | | C |
| ANISOU | 141 | CE | LYS | A | 406 | 5300 | 4695 | 5060 | 207 | 281 | 98 | | C |
| ATOM | 142 | NZ | LYS | A | 406 | −26.007 | −13.388 | 0.225 | 1.00 | 48.43 | | | N |
| ANISOU | 142 | NZ | LYS | A | 406 | 6314 | 5799 | 6287 | 277 | 433 | 117 | | N |
| ATOM | 143 | C | LYS | A | 406 | −20.777 | −10.898 | −2.584 | 1.00 | 15.69 | | | C |
| ANISOU | 143 | C | LYS | A | 406 | 2510 | 1639 | 1811 | −61 | −49 | 82 | | C |
| ATOM | 144 | O | LYS | A | 406 | −20.002 | −11.753 | −2.154 | 1.00 | 15.52 | | | O |
| ANISOU | 144 | O | LYS | A | 406 | 2471 | 1700 | 1726 | −106 | −57 | 62 | | O |
| ATOM | 145 | N | GLU | A | 407 | −20.713 | −9.619 | −2.225 | 1.00 | 13.73 | | | N |
| ANISOU | 145 | N | GLU | A | 407 | 2361 | 1278 | 1577 | −57 | −33 | 47 | | N |
| ATOM | 146 | CA | GLU | A | 407 | −19.765 | −9.164 | −1.210 | 1.00 | 15.97 | | | C |
| ANISOU | 146 | CA | GLU | A | 407 | 2729 | 1539 | 1801 | −121 | −40 | −51 | | C |
| ATOM | 147 | CB | GLU | A | 407 | −19.401 | −7.692 | −1.403 | 1.00 | 19.95 | | | C |
| ANISOU | 147 | CB | GLU | A | 407 | 3331 | 1884 | 2366 | −173 | −42 | −85 | | C |
| ATOM | 148 | CG | GLU | A | 407 | −18.398 | −7.175 | −0.373 | 1.00 | 26.13 | | | C |
| ANISOU | 148 | CG | GLU | A | 407 | 4183 | 2638 | 3108 | −262 | −76 | −229 | | C |
| ATOM | 149 | CD | GLU | A | 407 | −17.629 | −5.938 | −0.855 | 1.00 | 44.99 | | | C |
| ANISOU | 149 | CD | GLU | A | 407 | 6625 | 4865 | 5602 | −385 | −83 | −256 | | C |
| ATOM | 150 | OE1 | GLU | A | 407 | −17.412 | −5.794 | −2.084 | 1.00 | 42.31 | | | O |
| ANISOU | 150 | OE1 | GLU | A | 407 | 6254 | 4492 | 5330 | −428 | −64 | −133 | | O |
| ATOM | 151 | OE2 | GLU | A | 407 | −17.240 | −5.110 | 0.002 | 1.00 | 50.02 | | | O |
| ANISOU | 151 | OE2 | GLU | A | 407 | 7347 | 5404 | 6254 | −439 | −99 | −405 | | O |
| ATOM | 152 | C | GLU | A | 407 | −20.369 | −9.384 | 0.171 | 1.00 | 17.76 | | | C |
| ANISOU | 152 | C | GLU | A | 407 | 2999 | 1782 | 1965 | −31 | 23 | −119 | | C |
| ATOM | 153 | O | GLU | A | 407 | −21.462 | −8.890 | 0.459 | 1.00 | 17.74 | | | O |
| ANISOU | 153 | O | GLU | A | 407 | 3026 | 1704 | 2009 | 69 | 98 | −121 | | O |
| ATOM | 154 | N | LEU | A | 408 | −19.665 | −10.124 | 1.025 | 1.00 | 13.96 | | | N |
| ANISOU | 154 | N | LEU | A | 408 | 2525 | 1406 | 1371 | −45 | 3 | −164 | | N |
| ATOM | 155 | CA | LEU | A | 408 | −20.178 | −10.397 | 2.361 | 1.00 | 14.58 | | | C |
| ANISOU | 155 | CA | LEU | A | 408 | 2673 | 1516 | 1350 | 62 | 81 | −208 | | C |
| ATOM | 156 | CB | LEU | A | 408 | −19.684 | −11.757 | 2.847 | 1.00 | 14.10 | | | C |
| ANISOU | 156 | CB | LEU | A | 408 | 2578 | 1595 | 1184 | 83 | 81 | −166 | | C |
| ATOM | 157 | CG | LEU | A | 408 | −20.045 | −12.857 | 1.852 | 1.00 | 13.10 | | | C |
| ANISOU | 157 | CG | LEU | A | 408 | 2320 | 1498 | 1159 | 63 | 107 | −50 | | C |
| ATOM | 158 | CD1 | LEU | A | 408 | −19.528 | −14.195 | 2.335 | 1.00 | 15.19 | | | C |
| ANISOU | 158 | CD1 | LEU | A | 408 | 2569 | 1863 | 1338 | 94 | 126 | −0 | | C |
| ATOM | 159 | CD2 | LEU | A | 408 | −21.562 | −12.896 | 1.620 | 1.00 | 12.95 | | | C |
| ANISOU | 159 | CD2 | LEU | A | 408 | 2244 | 1412 | 1264 | 129 | 218 | 1 | | C |
| ATOM | 160 | C | LEU | A | 408 | −19.802 | −9.332 | 3.380 | 1.00 | 22.39 | | | C |
| ANISOU | 160 | C | LEU | A | 408 | 3805 | 2451 | 2250 | 72 | 56 | −356 | | C |
| ATOM | 161 | O | LEU | A | 408 | −20.494 | −9.195 | 4.393 | 1.00 | 21.65 | | | O |
| ANISOU | 161 | O | LEU | A | 408 | 3804 | 2347 | 2076 | 190 | 148 | −401 | | O |
| ATOM | 162 | N | GLY | A | 409 | −18.744 | −8.580 | 3.145 | 1.00 | 17.85 | | | N |
| ANISOU | 162 | N | GLY | A | 409 | 3250 | 1836 | 1696 | −49 | −53 | −443 | | N |
| ATOM | 163 | CA | GLY | A | 409 | −18.303 | −7.571 | 4.078 | 1.00 | 18.51 | | | C |
| ANISOU | 163 | CA | GLY | A | 409 | 3461 | 1856 | 1716 | −66 | −101 | −623 | | C |
| ATOM | 164 | C | GLY | A | 409 | −16.796 | −7.581 | 4.172 | 1.00 | 19.02 | | | C |
| ANISOU | 164 | C | GLY | A | 409 | 3478 | 2001 | 1749 | −202 | −255 | −719 | | C |
| ATOM | 165 | O | GLY | A | 409 | −16.111 | −8.293 | 3.442 | 1.00 | 17.88 | | | O |
| ANISOU | 165 | O | GLY | A | 409 | 3204 | 1948 | 1643 | −275 | −302 | −629 | | O |
| ATOM | 166 | N | THR | A | 410 | −16.281 | −6.783 | 5.101 | 1.00 | 24.11 | | | N |
| ANISOU | 166 | N | THR | A | 410 | 4220 | 2615 | 2327 | −231 | −336 | −920 | | N |
| ATOM | 167 | CA | THR | A | 410 | −14.848 | −6.612 | 5.297 | 1.00 | 29.32 | | | C |
| ANISOU | 167 | CA | THR | A | 410 | 4810 | 3346 | 2983 | −372 | −505 | −1058 | | C |
| ATOM | 168 | CB | THR | A | 410 | −14.435 | −5.153 | 5.080 | 1.00 | 35.35 | | | C |
| ANISOU | 168 | CB | THR | A | 410 | 5602 | 3903 | 3928 | −531 | −532 | −1206 | | C |
| ATOM | 169 | OG1 | THR | A | 410 | −14.654 | −4.785 | 3.712 | 1.00 | 33.56 | | | O |
| ANISOU | 169 | OG1 | THR | A | 410 | 5329 | 3516 | 3904 | −619 | −434 | −1050 | | O |
| ATOM | 170 | CG2 | THR | A | 410 | −12.973 | −4.953 | 5.430 | 1.00 | 38.61 | | | C |
| ANISOU | 170 | CG2 | THR | A | 410 | 5855 | 4426 | 4390 | −661 | −685 | −1357 | | C |
| ATOM | 171 | C | THR | A | 410 | −14.473 | −7.055 | 6.702 | 1.00 | 29.71 | | | C |
| ANISOU | 171 | C | THR | A | 410 | 4883 | 3593 | 2812 | −261 | −590 | −1172 | | C |
| ATOM | 172 | O | THR | A | 410 | −15.059 | −6.582 | 7.682 | 1.00 | 27.16 | | | O |
| ANISOU | 172 | O | THR | A | 410 | 4680 | 3251 | 2390 | −158 | −541 | −1266 | | O |
| ATOM | 173 | N | GLY | A | 411 | −13.513 | −7.970 | 6.792 | 1.00 | 33.07 | | | N |
| ANISOU | 173 | N | GLY | A | 411 | 5173 | 4220 | 3171 | −271 | −691 | −1136 | | N |
| ATOM | 174 | CA | GLY | A | 411 | −12.925 | −8.377 | 8.047 | 1.00 | 38.17 | | | C |
| ANISOU | 174 | CA | GLY | A | 411 | 5807 | 5068 | 3629 | −194 | −772 | −1201 | | C |
| ATOM | 175 | C | GLY | A | 411 | −11.546 | −7.786 | 8.244 | 1.00 | 44.23 | | | C |
| ANISOU | 175 | C | GLY | A | 411 | 6463 | 5888 | 4456 | −366 | −935 | −1355 | | C |
| ATOM | 176 | O | GLY | A | 411 | −11.180 | −6.769 | 7.641 | 1.00 | 45.77 | | | O |
| ANISOU | 176 | O | GLY | A | 411 | 6608 | 5935 | 4845 | −549 | −953 | −1460 | | O |
| ATOM | 177 | N | GLN | A | 412 | −10.759 | −8.441 | 9.101 | 1.00 | 51.02 | | | N |
| ANISOU | 177 | N | GLN | A | 412 | 7288 | 6937 | 5161 | −304 | −1050 | −1349 | | N |
| ATOM | 178 | CA | GLN | A | 412 | −9.447 | −7.895 | 9.436 | 1.00 | 56.93 | | | C |
| ANISOU | 178 | CA | GLN | A | 412 | 7945 | 7731 | 5955 | −446 | −1233 | −1492 | | C |
| ATOM | 179 | CB | GLN | A | 412 | −8.950 | −8.469 | 10.764 | 1.00 | 64.38 | | | C |
| ANISOU | 179 | CB | GLN | A | 412 | 8918 | 8875 | 6670 | −298 | −1353 | −1520 | | C |
| ATOM | 180 | CG | GLN | A | 412 | −8.009 | −9.659 | 10.638 | 1.00 | 65.80 | | | C |
| ANISOU | 180 | CG | GLN | A | 412 | 8944 | 9229 | 6829 | −229 | −1446 | −1388 | | C |
| ATOM | 181 | CD | GLN | A | 412 | −7.214 | −9.897 | 11.914 | 1.00 | 71.15 | | | C |
| ANISOU | 181 | CD | GLN | A | 412 | 9621 | 10088 | 7323 | −123 | −1616 | −1472 | | C |
| ATOM | 182 | OE1 | GLN | A | 412 | −7.456 | −10.862 | 12.642 | 1.00 | 78.01 | | | O |
| ANISOU | 182 | OE1 | GLN | A | 412 | 10564 | 11078 | 7997 | 90 | −1588 | −1360 | | O |
| ATOM | 183 | NE2 | GLN | A | 412 | −6.261 | −9.011 | 12.190 | 1.00 | 65.68 | | | N |
| ANISOU | 183 | NE2 | GLN | A | 412 | 8847 | 9408 | 6702 | −270 | −1793 | −1668 | | N |
| ATOM | 184 | C | GLN | A | 412 | −8.438 | −8.155 | 8.328 | 1.00 | 55.35 | | | C |
| ANISOU | 184 | C | GLN | A | 412 | 7540 | 7536 | 5953 | −586 | −1300 | −1419 | | C |
| ATOM | 185 | O | GLN | A | 412 | −7.614 | −7.287 | 8.019 | 1.00 | 62.31 | | | O |
| ANISOU | 185 | O | GLN | A | 412 | 8331 | 8332 | 7014 | −774 | −1387 | −1528 | | O |
| ATOM | 186 | N | PHE | A | 413 | −8.485 | −9.341 | 7.723 | 1.00 | 43.88 | | | N |
| ANISOU | 186 | N | PHE | A | 413 | 6012 | 6169 | 4489 | −492 | −1250 | −1233 | | N |
| ATOM | 187 | CA | PHE | A | 413 | −7.672 | −9.628 | 6.553 | 1.00 | 44.77 | | | C |
| ANISOU | 187 | CA | PHE | A | 413 | 5933 | 6277 | 4799 | −598 | −1278 | −1154 | | C |
| ATOM | 188 | CB | PHE | A | 413 | −7.589 | −11.137 | 6.318 | 1.00 | 48.52 | | | C |
| ANISOU | 188 | CB | PHE | A | 413 | 6345 | 6899 | 5192 | −438 | −1245 | −973 | | C |
| ATOM | 189 | CG | PHE | A | 413 | −6.763 | −11.868 | 7.338 | 1.00 | 51.48 | | | C |
| ANISOU | 189 | CG | PHE | A | 413 | 6672 | 7462 | 5425 | −309 | −1365 | −971 | | C |
| ATOM | 190 | CD1 | PHE | A | 413 | −5.378 | −11.766 | 7.325 | 1.00 | 54.09 | | | C |
| ANISOU | 190 | CD1 | PHE | A | 413 | 6806 | 7885 | 5859 | −386 | −1515 | −1039 | | C |
| ATOM | 191 | CE1 | PHE | A | 413 | −4.615 | −12.438 | 8.253 | 1.00 | 55.64 | | | C |
| ANISOU | 191 | CE1 | PHE | A | 413 | 6955 | 8263 | 5924 | −250 | −1639 | −1041 | | C |
| ATOM | 192 | CZ | PHE | A | 413 | −5.232 | −13.228 | 9.204 | 1.00 | 53.60 | | | C |
| ANISOU | 192 | CZ | PHE | A | 413 | 6859 | 8078 | 5429 | −33 | −1597 | −962 | | C |
| ATOM | 193 | CE2 | PHE | A | 413 | −6.615 | −13.341 | 9.224 | 1.00 | 48.01 | | | C |
| ANISOU | 193 | CE2 | PHE | A | 413 | 6342 | 7266 | 4634 | 34 | −1424 | −883 | | C |
| ATOM | 194 | CD2 | PHE | A | 413 | −7.369 | −12.669 | 8.295 | 1.00 | 44.91 | | | C |
| ANISOU | 194 | CD2 | PHE | A | 413 | 5981 | 6710 | 4372 | −103 | −1316 | −893 | | C |
| ATOM | 195 | C | PHE | A | 413 | −8.197 | −8.964 | 5.290 | 1.00 | 36.95 | | | C |
| ANISOU | 195 | C | PHE | A | 413 | 4944 | 5080 | 4017 | −728 | −1169 | −1130 | | C |
| ATOM | 196 | O | PHE | A | 413 | −7.521 | −9.021 | 4.257 | 1.00 | 41.22 | | | O |
| ANISOU | 196 | O | PHE | A | 413 | 5306 | 5600 | 4757 | −823 | −1159 | −1081 | | O |
| ATOM | 197 | N | GLY | A | 414 | −9.380 | −8.354 | 5.335 | 1.00 | 29.48 | | | N |
| ANISOU | 197 | N | GLY | A | 414 | 4162 | 3997 | 3042 | −718 | −1050 | −1164 | | N |
| ATOM | 198 | CA | GLY | A | 414 | −9.929 | −7.751 | 4.140 | 1.00 | 29.38 | | | C |
| ANISOU | 198 | CA | GLY | A | 414 | 4131 | 3785 | 3245 | −793 | −930 | −1131 | | C |
| ATOM | 199 | C | GLY | A | 414 | −11.301 | −8.261 | 3.746 | 1.00 | 28.28 | | | C |
| ANISOU | 199 | C | GLY | A | 414 | 4143 | 3587 | 3016 | −684 | −779 | −957 | | C |
| ATOM | 200 | O | GLY | A | 414 | −12.074 | −8.755 | 4.579 | 1.00 | 23.99 | | | O |
| ANISOU | 200 | O | GLY | A | 414 | 3721 | 3106 | 2289 | −512 | −757 | −947 | | O |
| ATOM | 201 | N | VAL | A | 415 | −11.600 | −8.153 | 2.467 | 1.00 | 20.00 | | | N |
| ANISOU | 201 | N | VAL | A | 415 | 3056 | 2423 | 2120 | −747 | −654 | −802 | | N |
| ATOM | 202 | CA | VAL | A | 415 | −12.939 | −8.396 | 1.957 | 1.00 | 18.63 | | | C |
| ANISOU | 202 | CA | VAL | A | 415 | 2977 | 2168 | 1934 | −635 | −513 | −649 | | C |
| ATOM | 203 | CB | VAL | A | 415 | −13.107 | −7.704 | 0.589 | 1.00 | 20.48 | | | C |
| ANISOU | 203 | CB | VAL | A | 415 | 3197 | 2237 | 2348 | −731 | −414 | −540 | | C |
| ATOM | 204 | CG1 | VAL | A | 415 | −14.463 | −8.043 | −0.013 | 1.00 | 28.18 | | | C |
| ANISOU | 204 | CG1 | VAL | A | 415 | 4233 | 3166 | 3306 | −602 | −307 | −390 | | C |
| ATOM | 205 | CG2 | VAL | A | 415 | −12.952 | −6.217 | 0.744 | 1.00 | 22.00 | | | C |
| ANISOU | 205 | CG2 | VAL | A | 415 | 3467 | 2224 | 2669 | −847 | −409 | −661 | | C |
| ATOM | 206 | C | VAL | A | 415 | −13.199 | −9.893 | 1.853 | 1.00 | 17.19 | | | C |
| ANISOU | 206 | C | VAL | A | 415 | 2744 | 2145 | 1644 | −507 | −485 | −513 | | C |
| ATOM | 207 | O | VAL | A | 415 | −12.300 | −10.676 | 1.524 | 1.00 | 20.78 | | | O |
| ANISOU | 207 | O | VAL | A | 415 | 3073 | 2728 | 2096 | −532 | −532 | −469 | | O |
| ATOM | 208 | N | VAL | A | 416 | −14.440 | −10.292 | 2.112 | 1.00 | 15.73 | | | N |
| ANISOU | 208 | N | VAL | A | 416 | 2647 | 1936 | 1393 | −373 | −395 | −445 | | N |
| ATOM | 209 | CA | VAL | A | 416 | −14.880 | −11.670 | 1.950 | 1.00 | 15.05 | | | C |
| ANISOU | 209 | CA | VAL | A | 416 | 2521 | 1945 | 1251 | −268 | −336 | −314 | | C |
| ATOM | 210 | CB | VAL | A | 416 | −15.296 | −12.303 | 3.292 | 1.00 | 14.96 | | | C |
| ANISOU | 210 | CB | VAL | A | 416 | 2599 | 2015 | 1070 | −118 | −312 | −330 | | C |
| ATOM | 211 | CG1 | VAL | A | 416 | −15.771 | −13.743 | 3.070 | 1.00 | 16.80 | | | C |
| ANISOU | 211 | CG1 | VAL | A | 416 | 2790 | 2301 | 1291 | −28 | −220 | −183 | | C |
| ATOM | 212 | CG2 | VAL | A | 416 | −14.138 | −12.269 | 4.248 | 1.00 | 16.47 | | | C |
| ANISOU | 212 | CG2 | VAL | A | 416 | 2796 | 2337 | 1127 | −110 | −452 | −447 | | C |
| ATOM | 213 | C | VAL | A | 416 | −16.036 | −11.681 | 0.963 | 1.00 | 15.82 | | | C |
| ANISOU | 213 | C | VAL | A | 416 | 2616 | 1940 | 1454 | −250 | −231 | −206 | | C |
| ATOM | 214 | O | VAL | A | 416 | −16.965 | −10.871 | 1.075 | 1.00 | 14.01 | | | O |
| ANISOU | 214 | O | VAL | A | 416 | 2461 | 1596 | 1264 | −218 | −177 | −225 | | O |
| ATOM | 215 | N | LYS | A | 417 | −15.977 | −12.588 | −0.006 | 1.00 | 12.07 | | | N |
| ANISOU | 215 | N | LYS | A | 417 | 2053 | 1512 | 1022 | −258 | −213 | −105 | | N |
| ATOM | 216 | CA | LYS | A | 417 | −17.016 | −12.683 | −1.017 | 1.00 | 17.36 | | | C |
| ANISOU | 216 | CA | LYS | A | 417 | 2701 | 2117 | 1778 | −237 | −153 | −23 | | C |
| ATOM | 217 | CB | LYS | A | 417 | −16.488 | −12.281 | −2.396 | 1.00 | 11.70 | | | C |
| ANISOU | 217 | CB | LYS | A | 417 | 1943 | 1377 | 1125 | −321 | −179 | 23 | | C |
| ATOM | 218 | CG | LYS | A | 417 | −15.866 | −10.892 | −2.400 | 1.00 | 17.54 | | | C |
| ANISOU | 218 | CG | LYS | A | 417 | 2729 | 2024 | 1910 | −412 | −198 | −25 | | C |
| ATOM | 219 | CD | LYS | A | 417 | −16.240 | −10.109 | −3.640 | 1.00 | 17.62 | | | C |
| ANISOU | 219 | CD | LYS | A | 417 | 2769 | 1933 | 1994 | −427 | −162 | 58 | | C |
| ATOM | 220 | CE | LYS | A | 417 | −15.429 | −8.840 | −3.736 | 1.00 | 26.18 | | | C |
| ANISOU | 220 | CE | LYS | A | 417 | 3894 | 2899 | 3153 | −541 | −150 | 31 | | C |
| ATOM | 221 | NZ | LYS | A | 417 | −15.778 | −8.073 | −4.957 | 1.00 | 32.66 | | | N |
| ANISOU | 221 | NZ | LYS | A | 417 | 4772 | 3610 | 4030 | −530 | −90 | 148 | | N |
| ATOM | 222 | C | LYS | A | 417 | −17.565 | −14.099 | −1.055 | 1.00 | 14.71 | | | C |
| ANISOU | 222 | C | LYS | A | 417 | 2315 | 1838 | 1437 | −168 | −103 | 39 | | C |
| ATOM | 223 | O | LYS | A | 417 | −16.911 | −15.059 | −0.622 | 1.00 | 13.53 | | | O |
| ANISOU | 223 | O | LYS | A | 417 | 2145 | 1772 | 1225 | −146 | −108 | 50 | | O |
| ATOM | 224 | N | TYR | A | 418 | −18.799 | −14.196 | −1.554 | 1.00 | 12.42 | | | N |
| ANISOU | 224 | N | TYR | A | 418 | 1998 | 1493 | 1227 | −130 | −55 | 75 | | N |
| ATOM | 225 | CA | TYR | A | 418 | −19.440 | −15.457 | −1.891 | 1.00 | 9.92 | | | C |
| ANISOU | 225 | CA | TYR | A | 418 | 1607 | 1196 | 965 | −101 | −10 | 116 | | C |
| ATOM | 226 | CB | TYR | A | 418 | −20.901 | −15.474 | −1.435 | 1.00 | 10.43 | | | C |
| ANISOU | 226 | CB | TYR | A | 418 | 1646 | 1202 | 1113 | −37 | 78 | 123 | | C |
| ATOM | 227 | CG | TYR | A | 418 | −21.759 | −16.491 | −2.161 | 1.00 | 13.47 | | | C |
| ANISOU | 227 | CG | TYR | A | 418 | 1916 | 1580 | 1623 | −43 | 99 | 139 | | C |
| ATOM | 228 | CD1 | TYR | A | 418 | −21.592 | −17.849 | −1.950 | 1.00 | 10.36 | | | C |
| ANISOU | 228 | CD1 | TYR | A | 418 | 1492 | 1192 | 1253 | −52 | 159 | 159 | | C |
| ATOM | 229 | CE1 | TYR | A | 418 | −22.359 | −18.776 | −2.615 | 1.00 | 14.09 | | | C |
| ANISOU | 229 | CE1 | TYR | A | 418 | 1855 | 1632 | 1866 | −81 | 175 | 143 | | C |
| ATOM | 230 | CZ | TYR | A | 418 | −23.319 | −18.351 | −3.515 | 1.00 | 19.98 | | | C |
| ANISOU | 230 | CZ | TYR | A | 418 | 2504 | 2375 | 2715 | −91 | 103 | 100 | | C |
| ATOM | 231 | OH | TYR | A | 418 | −24.095 | −19.268 | −4.186 | 1.00 | 14.22 | | | O |
| ANISOU | 231 | OH | TYR | A | 418 | 1645 | 1623 | 2135 | −130 | 88 | 49 | | O |
| ATOM | 232 | CE2 | TYR | A | 418 | −23.511 | −17.010 | −3.741 | 1.00 | 19.16 | | | C |
| ANISOU | 232 | CE2 | TYR | A | 418 | 2428 | 2282 | 2567 | −54 | 38 | 102 | | C |
| ATOM | 233 | CD2 | TYR | A | 418 | −22.735 | −16.085 | −3.071 | 1.00 | 12.57 | | | C |
| ANISOU | 233 | CD2 | TYR | A | 418 | 1719 | 1446 | 1610 | −36 | 50 | 124 | | C |
| ATOM | 234 | C | TYR | A | 418 | −19.372 | −15.660 | −3.404 | 1.00 | 12.33 | | | C |
| ANISOU | 234 | C | TYR | A | 418 | 1853 | 1517 | 1315 | −143 | −72 | 132 | | C |
| ATOM | 235 | O | TYR | A | 418 | −19.584 | −14.720 | −4.173 | 1.00 | 13.54 | | | O |
| ANISOU | 235 | O | TYR | A | 418 | 2020 | 1641 | 1484 | −152 | −116 | 139 | | O |
| ATOM | 236 | N | GLY | A | 419 | −19.084 | −16.881 | −3.827 | 1.00 | 9.79 | | | N |
| ANISOU | 236 | N | GLY | A | 419 | 1484 | 1236 | 1002 | −152 | −68 | 138 | | N |
| ATOM | 237 | CA | GLY | A | 419 | −18.993 | −17.140 | −5.249 | 1.00 | 9.90 | | | C |
| ANISOU | 237 | CA | GLY | A | 419 | 1462 | 1277 | 1023 | −176 | −127 | 133 | | C |
| ATOM | 238 | C | GLY | A | 419 | −19.124 | −18.612 | −5.551 | 1.00 | 9.39 | | | C |
| ANISOU | 238 | C | GLY | A | 419 | 1346 | 1217 | 1005 | −173 | −104 | 109 | | C |
| ATOM | 239 | O | GLY | A | 419 | −19.487 | −19.407 | −4.684 | 1.00 | 9.29 | | | O |
| ANISOU | 239 | O | GLY | A | 419 | 1317 | 1163 | 1050 | −155 | −26 | 115 | | O |
| ATOM | 240 | N | LYS | A | 420 | −18.821 | −18.966 | −6.807 | 1.00 | 11.92 | | | N |
| ANISOU | 240 | N | LYS | A | 420 | 1656 | 1577 | 1297 | −184 | −159 | 82 | | N |
| ATOM | 241 | CA | LYS | A | 420 | −18.843 | −20.340 | −7.286 | 1.00 | 12.65 | | | C |
| ANISOU | 241 | CA | LYS | A | 420 | 1715 | 1658 | 1432 | −186 | −146 | 29 | | C |
| ATOM | 242 | CB | LYS | A | 420 | −19.850 | −20.550 | −8.445 | 1.00 | 14.10 | | | C |
| ANISOU | 242 | CB | LYS | A | 420 | 1850 | 1845 | 1661 | −188 | −233 | −56 | | C |
| ATOM | 243 | CG | LYS | A | 420 | −21.314 | −20.262 | −8.095 | 1.00 | 19.61 | | | C |
| ANISOU | 243 | CG | LYS | A | 420 | 2463 | 2498 | 2492 | −190 | −251 | −81 | | C |
| ATOM | 244 | CD | LYS | A | 420 | −21.933 | −21.361 | −7.257 | 1.00 | 16.55 | | | C |
| ANISOU | 244 | CD | LYS | A | 420 | 2004 | 2014 | 2271 | −223 | −148 | −106 | | C |
| ATOM | 245 | CE | LYS | A | 420 | −23.444 | −21.227 | −7.261 | 1.00 | 21.35 | | | C |
| ANISOU | 245 | CE | LYS | A | 420 | 2477 | 2591 | 3044 | −236 | −173 | −159 | | C |
| ATOM | 246 | NZ | LYS | A | 420 | −24.085 | −22.266 | −6.414 | 1.00 | 19.46 | | | N |
| ANISOU | 246 | NZ | LYS | A | 420 | 2157 | 2236 | 3000 | −284 | −32 | −167 | | N |
| ATOM | 247 | C | LYS | A | 420 | −17.460 | −20.725 | −7.784 | 1.00 | 12.28 | | | C |
| ANISOU | 247 | C | LYS | A | 420 | 1705 | 1670 | 1289 | −178 | −136 | 40 | | C |
| ATOM | 248 | O | LYS | A | 420 | −16.716 | −19.891 | −8.302 | 1.00 | 14.96 | | | O |
| ANISOU | 248 | O | LYS | A | 420 | 2073 | 2072 | 1538 | −185 | −161 | 75 | | O |
| ATOM | 249 | N | TRP | A | 421 | −17.130 | −22.002 | −7.634 | 1.00 | 13.79 | | | N |
| ANISOU | 249 | N | TRP | A | 421 | 1890 | 1829 | 1519 | −160 | −80 | 18 | | N |
| ATOM | 250 | CA | TRP | A | 421 | −15.924 | −22.587 | −8.208 | 1.00 | 13.50 | | | C |
| ANISOU | 250 | CA | TRP | A | 421 | 1872 | 1843 | 1414 | −129 | −58 | 15 | | C |
| ATOM | 251 | CB | TRP | A | 421 | −15.139 | −23.370 | −7.144 | 1.00 | 11.26 | | | C |
| ANISOU | 251 | CB | TRP | A | 421 | 1589 | 1542 | 1148 | −79 | 21 | 70 | | C |
| ATOM | 252 | CG | TRP | A | 421 | −13.882 | −24.017 | −7.639 | 1.00 | 12.28 | | | C |
| ANISOU | 252 | CG | TRP | A | 421 | 1716 | 1724 | 1228 | −23 | 54 | 73 | | C |
| ATOM | 253 | CD1 | TRP | A | 421 | −12.662 | −23.424 | −7.808 | 1.00 | 12.25 | | | C |
| ANISOU | 253 | CD1 | TRP | A | 421 | 1676 | 1832 | 1144 | −14 | 46 | 114 | | C |
| ATOM | 254 | NE1 | TRP | A | 421 | −11.751 | −24.345 | −8.269 | 1.00 | 15.20 | | | N |
| ANISOU | 254 | NE1 | TRP | A | 421 | 2038 | 2230 | 1507 | 59 | 103 | 105 | | N |
| ATOM | 255 | CE2 | TRP | A | 421 | −12.376 | −25.556 | −8.406 | 1.00 | 18.72 | | | C |
| ANISOU | 255 | CE2 | TRP | A | 421 | 2533 | 2559 | 2022 | 96 | 147 | 51 | | C |
| ATOM | 256 | CD2 | TRP | A | 421 | −13.717 | −25.388 | −8.008 | 1.00 | 16.73 | | | C |
| ANISOU | 256 | CD2 | TRP | A | 421 | 2300 | 2216 | 1839 | 33 | 118 | 29 | | C |
| ATOM | 257 | CE3 | TRP | A | 421 | −14.578 | −26.489 | −8.061 | 1.00 | 18.67 | | | C |
| ANISOU | 257 | CE3 | TRP | A | 421 | 2573 | 2317 | 2203 | 30 | 163 | −36 | | C |
| ATOM | 258 | CZ3 | TRP | A | 421 | −14.075 | −27.705 | −8.496 | 1.00 | 17.92 | | | C |
| ANISOU | 258 | CZ3 | TRP | A | 421 | 2512 | 2154 | 2144 | 94 | 232 | −82 | | C |
| ATOM | 259 | CH2 | TRP | A | 421 | −12.739 | −27.839 | −8.882 | 1.00 | 19.02 | | | C |
| ANISOU | 259 | CH2 | TRP | A | 421 | 2648 | 2386 | 2192 | 180 | 260 | −56 | | C |
| ATOM | 260 | CZ2 | TRP | A | 421 | −11.876 | −26.780 | −8.845 | 1.00 | 18.90 | | | C |
| ANISOU | 260 | CZ2 | TRP | A | 421 | 2580 | 2533 | 2070 | 180 | 220 | 13 | | C |
| ATOM | 261 | C | TRP | A | 421 | −16.359 | −23.480 | −9.363 | 1.00 | 14.46 | | | C |
| ANISOU | 261 | C | TRP | A | 421 | 2003 | 1934 | 1557 | −124 | −81 | −93 | | C |
| ATOM | 262 | O | TRP | A | 421 | −17.257 | −24.316 | −9.196 | 1.00 | 11.78 | | | O |
| ANISOU | 262 | O | TRP | A | 421 | 1639 | 1495 | 1343 | −144 | −67 | −158 | | O |
| ATOM | 263 | N | ARG | A | 422 | −15.753 | −23.268 | −10.537 | 1.00 | 14.53 | | | N |
| ANISOU | 263 | N | ARG | A | 422 | 1558 | 3154 | 807 | −257 | 33 | −118 | | N |
| ATOM | 264 | CA | ARG | A | 422 | −16.102 | −23.985 | −11.767 | 1.00 | 15.69 | | | C |
| ANISOU | 264 | CA | ARG | A | 422 | 1659 | 3415 | 886 | −265 | 35 | −214 | | C |
| ATOM | 265 | CB | ARG | A | 422 | −15.471 | −25.379 | −11.803 | 1.00 | 22.46 | | | C |
| ANISOU | 265 | CB | ARG | A | 422 | 2550 | 4235 | 1750 | −234 | 71 | −395 | | C |
| ATOM | 266 | CG | ARG | A | 422 | −13.953 | −25.335 | −11.742 | 1.00 | 16.19 | | | C |
| ANISOU | 266 | CG | ARG | A | 422 | 1738 | 3462 | 952 | −186 | 111 | −388 | | C |
| ATOM | 267 | CD | ARG | A | 422 | −13.287 | −26.635 | −12.171 | 1.00 | 23.88 | | | C |
| ANISOU | 267 | CD | ARG | A | 422 | 2719 | 4436 | 1916 | −138 | 145 | −555 | | C |
| ATOM | 268 | NE | ARG | A | 422 | −11.838 | −26.450 | −12.225 | 1.00 | 21.49 | | | N |
| ANISOU | 268 | NE | ARG | A | 422 | 2374 | 4192 | 1598 | −89 | 181 | −527 | | N |
| ATOM | 269 | CZ | ARG | A | 422 | −10.957 | −27.409 | −12.488 | 1.00 | 29.39 | | | C |
| ANISOU | 269 | Cz | ARG | A | 422 | 3372 | 5199 | 2595 | −25 | 216 | −647 | | C |
| ATOM | 270 | NH1 | ARG | A | 422 | −11.373 | −28.645 | −12.700 | 1.00 | 36.31 | | | N |
| ANISOU | 270 | NH1 | ARG | A | 422 | 4301 | 6002 | 3492 | −4 | 220 | −804 | | N |
| ATOM | 271 | NH2 | ARG | A | 422 | −9.653 | −27.129 | −12.528 | 1.00 | 26.68 | | | N |
| ANISOU | 271 | NH2 | ARG | A | 422 | 2972 | 4930 | 2234 | 17 | 245 | −605 | | N |
| ATOM | 272 | C | ARG | A | 422 | −17.610 | −24.057 | −11.954 | 1.00 | 22.34 | | | C |
| ANISOU | 272 | C | ARG | A | 422 | 2496 | 4269 | 1722 | −301 | −3 | −223 | | C |
| ATOM | 273 | O | ARG | A | 422 | −18.165 | −25.093 | −12.315 | 1.00 | 16.42 | | | O |
| ANISOU | 273 | O | ARG | A | 422 | 1753 | 3525 | 959 | −320 | −3 | −359 | | O |
| ATOM | 274 | N | GLY | A | 423 | −18.280 | −22.937 | −11.679 | 1.00 | 15.43 | | | N |
| ANISOU | 274 | N | GLY | A | 423 | 1607 | 3389 | 866 | −311 | −35 | −75 | | N |
| ATOM | 275 | CA | GLY | A | 423 | −19.676 | −22.756 | −12.017 | 1.00 | 15.80 | | | C |
| ANISOU | 275 | CA | GLY | A | 423 | 1620 | 3487 | 896 | −334 | −72 | −49 | | C |
| ATOM | 276 | C | GLY | A | 423 | −20.677 | −23.450 | −11.117 | 1.00 | 21.86 | | | C |
| ANISOU | 276 | C | GLY | A | 423 | 2439 | 4153 | 1714 | −357 | −90 | −136 | | C |
| ATOM | 277 | O | GLY | A | 423 | −21.856 | −23.065 | −11.119 | 1.00 | 22.67 | | | O |
| ANISOU | 277 | O | GLY | A | 423 | 2510 | 4287 | 1816 | −370 | −123 | −82 | | O |
| ATOM | 278 | N | GLN | A | 424 | −20.261 | −24.450 | −10.348 | 1.00 | 14.69 | | | N |
| ANISOU | 278 | N | GLN | A | 424 | 1604 | 3125 | 854 | −361 | −68 | −263 | | N |
| ATOM | 279 | CA | GLN | A | 424 | −21.197 | −25.329 | −9.663 | 1.00 | 18.78 | | | C |
| ANISOU | 279 | CA | GLN | A | 424 | 2165 | 3549 | 1422 | −401 | −80 | −364 | | C |
| ATOM | 280 | CB | GLN | A | 424 | −20.979 | −26.784 | −10.105 | 1.00 | 25.01 | | | C |
| ANISOU | 280 | CB | GLN | A | 424 | 2982 | 4304 | 2216 | −421 | −54 | −540 | | C |
| ATOM | 281 | CG | GLN | A | 424 | −22.080 | −27.363 | −10.947 | 1.00 | 35.38 | | | C |
| ANISOU | 281 | CG | GLN | A | 424 | 4250 | 5702 | 3492 | −477 | −72 | −609 | | C |
| ATOM | 282 | CD | GLN | A | 424 | −21.669 | −28.655 | −11.617 | 1.00 | 40.94 | | | C |
| ANISOU | 282 | CD | GLN | A | 424 | 4978 | 6387 | 4190 | −486 | −47 | −772 | | C |
| ATOM | 283 | OE1 | GLN | A | 424 | −20.591 | −28.748 | −12.210 | 1.00 | 39.53 | | | O |
| ANISOU | 283 | OE1 | GLN | A | 424 | 4792 | 6253 | 3974 | −440 | −21 | −800 | | O |
| ATOM | 284 | NE2 | GLN | A | 424 | −22.528 | −29.665 | −11.524 | 1.00 | 45.16 | | | N |
| ANISOU | 284 | NE2 | GLN | A | 424 | 5539 | 6856 | 4764 | −547 | −55 | −877 | | N |
| ATOM | 285 | C | GLN | A | 424 | −21.121 | −25.287 | −8.148 | 1.00 | 20.96 | | | C |
| ANISOU | 285 | C | GLN | A | 424 | 2513 | 3619 | 1830 | −378 | −72 | −333 | | C |
| ATOM | 286 | O | GLN | A | 424 | −22.146 | −25.506 | −7.503 | 1.00 | 21.66 | | | O |
| ANISOU | 286 | O | GLN | A | 424 | 2614 | 3644 | 1970 | −407 | −88 | −341 | | O |
| ATOM | 287 | N | TYR | A | 425 | −19.957 | −25.023 | −7.559 | 1.00 | 12.66 | | | N |
| ANISOU | 287 | N | TYR | A | 425 | 1500 | 2470 | 839 | −327 | −45 | −294 | | N |
| ATOM | 288 | CA | TYR | A | 425 | −19.732 | −25.272 | −6.140 | 1.00 | 15.03 | | | C |
| ANISOU | 288 | CA | TYR | A | 425 | 1871 | 2577 | 1263 | −302 | −32 | −294 | | C |
| ATOM | 289 | CB | TYR | A | 425 | −18.448 | −26.089 | −5.956 | 1.00 | 14.69 | | | C |
| ANISOU | 289 | CB | TYR | A | 425 | 1870 | 2464 | 1246 | −265 | 5 | −374 | | C |
| ATOM | 290 | CG | TYR | A | 425 | −18.547 | −27.461 | −6.581 | 1.00 | 21.30 | | | C |
| ANISOU | 290 | CG | TYR | A | 425 | 2728 | 3314 | 2051 | −289 | 22 | −533 | | C |
| ATOM | 291 | CD1 | TYR | A | 425 | −19.176 | −28.494 | −5.907 | 1.00 | 29.53 | | | C |
| ANISOU | 291 | CD1 | TYR | A | 425 | 3831 | 4222 | 3167 | −318 | 24 | −611 | | C |
| ATOM | 292 | CE1 | TYR | A | 425 | −19.280 | −29.748 | −6.456 | 1.00 | 27.21 | | | C |
| ANISOU | 292 | CE1 | TYR | A | 425 | 3567 | 3911 | 2862 | −347 | 37 | −764 | | C |
| ATOM | 293 | CZ | TYR | A | 425 | −18.766 | −29.994 | −7.704 | 1.00 | 29.81 | | | C |
| ANISOU | 293 | CZ | TYR | A | 425 | 3860 | 4374 | 3091 | −338 | 49 | −856 | | C |
| ATOM | 294 | OH | TYR | A | 425 | −18.898 | −31.257 | −8.223 | 1.00 | 33.76 | | | O |
| ANISOU | 294 | OH | TYR | A | 425 | 4393 | 4817 | 3618 | −349 | 58 | −981 | | O |
| ATOM | 295 | CE2 | TYR | A | 425 | −18.140 | −28.988 | −8.416 | 1.00 | 25.97 | | | C |
| ANISOU | 295 | CE2 | TYR | A | 425 | 3303 | 4052 | 2512 | −306 | 50 | −777 | | C |
| ATOM | 296 | CD2 | TYR | A | 425 | −18.030 | −27.723 | −7.846 | 1.00 | 27.51 | | | C |
| ANISOU | 296 | CD2 | TYR | A | 425 | 3473 | 4246 | 2732 | −286 | 35 | −608 | | C |
| ATOM | 297 | C | TYR | A | 425 | −19.642 | −23.962 | −5.364 | 1.00 | 15.66 | | | C |
| ANISOU | 297 | C | TYR | A | 425 | 1948 | 2601 | 1400 | −268 | −46 | −153 | | C |
| ATOM | 298 | O | TYR | A | 425 | −18.810 | −23.108 | −5.691 | 1.00 | 14.19 | | | O |
| ANISOU | 298 | O | TYR | A | 425 | 1739 | 2457 | 1196 | −246 | −43 | −75 | | O |
| ATOM | 299 | N | ASP | A | 426 | −20.482 | −23.816 | −4.327 | 1.00 | 11.87 | | | N |
| ANISOU | 299 | N | ASP | A | 426 | 1493 | 2026 | 991 | −268 | −58 | −127 | | N |
| ATOM | 300 | CA | ASP | A | 426 | −20.431 | −22.627 | −3.482 | 1.00 | 10.88 | | | C |
| ANISOU | 300 | CA | ASP | A | 426 | 1375 | 1830 | 928 | −230 | −70 | −21 | | C |
| ATOM | 301 | CB | ASP | A | 426 | −21.513 | −22.678 | −2.395 | 1.00 | 10.27 | | | C |
| ANISOU | 301 | CB | ASP | A | 426 | 1316 | 1677 | 908 | −228 | −78 | −19 | | C |
| ATOM | 302 | CG | ASP | A | 426 | −22.909 | −22.530 | −2.957 | 1.00 | 20.41 | | | C |
| ANISOU | 302 | CG | ASP | A | 426 | 2543 | 3064 | 2146 | −253 | −103 | −3 | | C |
| ATOM | 303 | OD1 | ASP | A | 426 | −23.061 | −21.975 | −4.070 | 1.00 | 20.02 | | | O |
| ANISOU | 303 | OD1 | ASP | A | 426 | 2442 | 3138 | 2028 | −255 | −122 | 44 | | O |
| ATOM | 304 | OD2 | ASP | A | 426 | −23.847 | −22.965 | −2.274 | 1.00 | 23.77 | | | O |
| ANISOU | 304 | OD2 | ASP | A | 426 | 2970 | 3462 | 2598 | −271 | −103 | −30 | | O |
| ATOM | 305 | C | ASP | A | 426 | −19.079 | −22.520 | −2.795 | 1.00 | 13.34 | | | C |
| ANISOU | 305 | C | ASP | A | 426 | 1725 | 2052 | 1291 | −198 | −50 | −15 | | C |
| ATOM | 306 | O | ASP | A | 426 | −18.576 | −23.495 | −2.245 | 1.00 | 12.83 | | | O |
| ANISOU | 306 | O | ASP | A | 426 | 1701 | 1918 | 1256 | −190 | −29 | −87 | | O |
| ATOM | 307 | N | VAL | A | 427 | −18.515 | −21.319 | −2.770 | 1.00 | 9.27 | | | N |
| ANISOU | 307 | N | VAL | A | 427 | 1197 | 1533 | 793 | −181 | −60 | 77 | | N |
| ATOM | 308 | CA | VAL | A | 427 | −17.287 | −21.095 | −2.019 | 1.00 | 12.25 | | | C |
| ANISOU | 308 | CA | VAL | A | 427 | 1599 | 1835 | 1221 | −162 | −48 | 86 | | C |
| ATOM | 309 | CB | VAL | A | 427 | −16.016 | −21.189 | −2.898 | 1.00 | 12.20 | | | C |
| ANISOU | 309 | CB | VAL | A | 427 | 1559 | 1914 | 1162 | −169 | −29 | 86 | | C |
| ATOM | 310 | CG1 | VAL | A | 427 | −15.811 | −22.599 | −3.419 | 1.00 | 13.51 | | | C |
| ANISOU | 310 | CG1 | VAL | A | 427 | 1727 | 2127 | 1278 | −163 | −3 | −25 | | C |
| ATOM | 311 | CG2 | VAL | A | 427 | −16.084 | −20.193 | −4.046 | 1.00 | 12.39 | | | C |
| ANISOU | 311 | CG2 | VAL | A | 427 | 1530 | 2046 | 1130 | −194 | −41 | 182 | | C |
| ATOM | 312 | C | VAL | A | 427 | −17.342 | −19.728 | −1.365 | 1.00 | 12.98 | | | C |
| ANISOU | 312 | C | VAL | A | 427 | 1699 | 1858 | 1374 | −151 | −70 | 172 | | C |
| ATOM | 313 | O | VAL | A | 427 | −18.049 | −18.825 | −1.824 | 1.00 | 10.04 | | | O |
| ANISOU | 313 | O | VAL | A | 427 | 1305 | 1510 | 998 | −152 | −90 | 245 | | O |
| ATOM | 314 | N | ALA | A | 428 | −16.586 | −19.586 | −0.281 | 1.00 | 8.91 | | | N |
| ANISOU | 314 | N | ALA | A | 428 | 1213 | 1256 | 915 | −138 | −67 | 159 | | N |
| ATOM | 315 | CA | ALA | A | 428 | −16.247 | −18.285 | 0.273 | 1.00 | 10.89 | | | C |
| ANISOU | 315 | CA | ALA | A | 428 | 1473 | 1439 | 1226 | −139 | −86 | 220 | | C |
| ATOM | 316 | CB | ALA | A | 428 | −16.308 | −18.296 | 1.803 | 1.00 | 7.95 | | | C |
| ANISOU | 316 | CB | ALA | A | 428 | 1142 | 969 | 911 | −113 | −91 | 174 | | C |
| ATOM | 317 | C | ALA | A | 428 | −14.839 | −17.929 | −0.184 | 1.00 | 9.62 | | | C |
| ANISOU | 317 | C | ALA | A | 428 | 1284 | 1315 | 1055 | −170 | −80 | 254 | | C |
| ATOM | 318 | O | ALA | A | 428 | −13.976 | −18.806 | −0.283 | 1.00 | 13.68 | | | O |
| ANISOU | 318 | O | ALA | A | 428 | 1783 | 1878 | 1535 | −168 | −58 | 206 | | O |
| ATOM | 319 | N | ILE | A | 429 | −14.607 | −16.641 | −0.428 | 1.00 | 10.11 | | | N |
| ANISOU | 319 | N | ILE | A | 429 | 1338 | 1352 | 1152 | −199 | −97 | 341 | | N |
| ATOM | 320 | CA | ILE | A | 429 | −13.317 | −16.134 | −0.903 | 1.00 | 10.66 | | | C |
| ANISOU | 320 | CA | ILE | A | 429 | 1370 | 1464 | 1215 | −248 | −92 | 394 | | C |
| ATOM | 321 | CB | ILE | A | 429 | −13.391 | −15.665 | −2.357 | 1.00 | 12.09 | | | C |
| ANISOU | 321 | CB | ILE | A | 429 | 1507 | 1746 | 1341 | −278 | −88 | 494 | | C |
| ATOM | 322 | CG1 | ILE | A | 429 | −13.953 | −16.768 | −3.252 | 1.00 | 17.13 | | | C |
| ANISOU | 322 | CG1 | ILE | A | 429 | 2120 | 2508 | 1882 | −253 | −69 | 448 | | C |
| ATOM | 323 | CD1 | ILE | A | 429 | −15.161 | −16.343 | −4.012 | 1.00 | 25.29 | | | C |
| ANISOU | 323 | CD1 | ILE | A | 429 | 3143 | 3579 | 2887 | −244 | −87 | 514 | | C |
| ATOM | 324 | CG2 | ILE | A | 429 | −12.007 | −15.191 | −2.827 | 1.00 | 15.92 | | | C |
| ANISOU | 324 | CG2 | ILE | A | 429 | 1943 | 2292 | 1813 | −340 | −76 | 557 | | C |
| ATOM | 325 | C | ILE | A | 429 | −12.883 | −14.977 | −0.006 | 1.00 | 11.28 | | | C |
| ANISOU | 325 | C | ILE | A | 429 | 1475 | 1425 | 1385 | −277 | −116 | 422 | | C |
| ATOM | 326 | O | ILE | A | 429 | −13.523 | −13.919 | −0.002 | 1.00 | 14.92 | | | O |
| ANISOU | 326 | O | ILE | A | 429 | 1963 | 1803 | 1904 | −280 | −137 | 482 | | O |
| ATOM | 327 | N | LYS | A | 430 | −11.783 | −15.156 | 0.725 | 1.00 | 13.35 | | | N |
| ANISOU | 327 | N | LYS | A | 430 | 1728 | 1683 | 1661 | −297 | −114 | 376 | | N |
| ATOM | 328 | CA | LYS | A | 430 | −11.212 | −14.093 | 1.554 | 1.00 | 14.48 | | | C |
| ANISOU | 328 | CA | LYS | A | 430 | 1888 | 1731 | 1884 | −343 | −140 | 384 | | C |
| ATOM | 329 | CB | LYS | A | 430 | −10.768 | −14.629 | 2.918 | 1.00 | 16.97 | | | C |
| ANISOU | 329 | CB | LYS | A | 430 | 2216 | 2025 | 2208 | −319 | −147 | 284 | | C |
| ATOM | 330 | CG | LYS | A | 430 | −10.071 | −13.597 | 3.803 | 1.00 | 19.57 | | | C |
| ANISOU | 330 | CG | LYS | A | 430 | 2554 | 2278 | 2606 | −377 | −177 | 268 | | C |
| ATOM | 331 | CD | LYS | A | 430 | −10.481 | −13.700 | 5.276 | 1.00 | 20.11 | | | C |
| ANISOU | 331 | CD | LYS | A | 430 | 2665 | 2281 | 2695 | −333 | −195 | 172 | | C |
| ATOM | 332 | CE | LYS | A | 430 | −10.008 | −14.999 | 5.926 | 1.00 | 20.95 | | | C |
| ANISOU | 332 | CE | LYS | A | 430 | 2746 | 2476 | 2737 | −285 | −182 | 112 | | C |
| ATOM | 333 | NZ | LYS | A | 430 | −10.605 | −15.174 | 7.293 | 1.00 | 21.77 | | | N |
| ANISOU | 333 | NZ | LYS | A | 430 | 2891 | 2537 | 2845 | −236 | −195 | 36 | | N |
| ATOM | 334 | C | LYS | A | 430 | −10.040 | −13.475 | 0.798 | 1.00 | 13.88 | | | C |
| ANISOU | 334 | C | LYS | A | 430 | 1757 | 1714 | 1804 | −427 | −136 | 465 | | C |
| ATOM | 335 | O | LYS | A | 430 | −9.058 | −14.160 | 0.509 | 1.00 | 15.39 | | | O |
| ANISOU | 335 | O | LYS | A | 430 | 1889 | 2022 | 1935 | −439 | −115 | 450 | | O |
| ATOM | 336 | N | AMET | A | 431 | −10.135 | −12.187 | 0.488 | 0.75 | 15.44 | | | N |
| ANISOU | 336 | N | AMET | A | 431 | 1970 | 1828 | 2068 | −483 | −154 | 554 | | N |
| ATOM | 337 | CA | AMET | A | 431 | −9.068 | −11.474 | −0.211 | 0.75 | 13.88 | | | C |
| ANISOU | 337 | CA | AMET | A | 431 | 1721 | 1676 | 1877 | −583 | −151 | 652 | | C |
| ATOM | 338 | CB | AMET | A | 431 | −9.659 | −10.473 | −1.202 | 0.75 | 15.04 | | | C |
| ANISOU | 338 | CB | AMET | A | 431 | 1881 | 1777 | 2055 | −610 | −157 | 793 | | C |
| ATOM | 339 | CG | AMET | A | 431 | −10.541 | −11.132 | −2.245 | 0.75 | 19.99 | | | C |
| ANISOU | 339 | CG | AMET | A | 431 | 2490 | 2515 | 2589 | −544 | −137 | 830 | | C |
| ATOM | 340 | SD | AMET | A | 431 | −11.526 | −9.956 | −3.192 | 0.75 | 28.33 | | | S |
| ANISOU | 340 | SD | AMET | A | 431 | 3571 | 3509 | 3685 | −544 | −154 | 998 | | S |
| ATOM | 341 | CE | AMET | A | 431 | −10.858 | −10.164 | −4.843 | 0.75 | 31.51 | | | C |
| ANISOU | 341 | CE | AMET | A | 431 | 3876 | 4136 | 3961 | −585 | −122 | 1100 | | C |
| ATOM | 342 | C | AMET | A | 431 | −8.172 | −10.788 | 0.816 | 0.75 | 17.84 | | | C |
| ANISOU | 342 | C | AMET | A | 431 | 2228 | 2095 | 2457 | −656 | −177 | 611 | | C |
| ATOM | 343 | O | AMET | A | 431 | −8.562 | −9.787 | 1.426 | 0.75 | 18.38 | | | O |
| ANISOU | 343 | O | AMET | A | 431 | 2358 | 2000 | 2625 | −678 | −207 | 607 | | O |
| ATOM | 344 | N | BMET | A | 431 | −10.195 | −12.211 | 0.411 | 0.25 | 14.70 | | | N |
| ANISOU | 344 | N | BMET | A | 431 | 1876 | 1739 | 1970 | −480 | −153 | 558 | | N |
| ATOM | 345 | CA | BMET | A | 431 | −9.098 | −11.429 | −0.140 | 0.25 | 15.01 | | | C |
| ANISOU | 345 | CA | BMET | A | 431 | 1868 | 1808 | 2026 | −582 | −153 | 649 | | C |
| ATOM | 346 | CB | BMET | A | 431 | −9.618 | −10.243 | −0.953 | 0.25 | 16.53 | | | C |
| ANISOU | 346 | CB | BMET | A | 431 | 2083 | 1925 | 2273 | −620 | −164 | 787 | | C |
| ATOM | 347 | CG | BMET | A | 431 | −9.914 | −10.554 | −2.401 | 0.25 | 20.61 | | | C |
| ANISOU | 347 | CG | BMET | A | 431 | 2551 | 2584 | 2694 | −608 | −139 | 888 | | C |
| ATOM | 348 | SD | BMET | A | 431 | −11.338 | −11.637 | −2.572 | 0.25 | 23.03 | | | S |
| ANISOU | 348 | SD | BMET | A | 431 | 2880 | 2941 | 2928 | −486 | −131 | 816 | | S |
| ATOM | 349 | CE | BMET | A | 431 | −12.620 | −10.605 | −1.864 | 0.25 | 23.54 | | | C |
| ANISOU | 349 | CE | BMET | A | 431 | 3030 | 2801 | 3112 | −437 | −168 | 831 | | C |
| ATOM | 350 | C | BMET | A | 431 | −8.230 | −10.940 | 1.006 | 0.25 | 16.76 | | | C |
| ANISOU | 350 | C | BMET | A | 431 | 2096 | 1955 | 2316 | −642 | −178 | 587 | | C |
| ATOM | 351 | O | BMET | A | 431 | −8.707 | −10.245 | 1.908 | 0.25 | 18.53 | | | O |
| ANISOU | 351 | O | BMET | A | 431 | 2386 | 2028 | 2627 | −637 | −207 | 542 | | O |
| ATOM | 352 | N | ILE | A | 432 | −6.951 | −11.317 | 0.977 | 1.00 | 15.78 | | | N |
| ANISOU | 352 | N | ILE | A | 432 | 1896 | 1952 | 2147 | −693 | −166 | 577 | | N |
| ATOM | 353 | CA | ILE | A | 432 | −6.000 | −10.879 | 1.988 | 1.00 | 17.75 | | | C |
| ANISOU | 353 | CA | ILE | A | 432 | 2129 | 2169 | 2445 | −763 | −193 | 521 | | C |
| ATOM | 354 | CB | ILE | A | 432 | −4.870 | −11.906 | 2.146 | 1.00 | 16.73 | | | C |
| ANISOU | 354 | CB | ILE | A | 432 | 1911 | 2216 | 2230 | −750 | −175 | 471 | | C |
| ATOM | 355 | CG1 | ILE | A | 432 | −5.441 | −13.285 | 2.489 | 1.00 | 15.72 | | | C |
| ANISOU | 355 | CG1 | ILE | A | 432 | 1804 | 2132 | 2038 | −616 | −157 | 386 | | C |
| ATOM | 356 | CD1 | ILE | A | 432 | −6.350 | −13.273 | 3.687 | 1.00 | 18.23 | | | C |
| ANISOU | 356 | CD1 | ILE | A | 432 | 2206 | 2320 | 2401 | −560 | −184 | 302 | | C |
| ATOM | 357 | CG2 | ILE | A | 432 | −3.865 | −11.449 | 3.173 | 1.00 | 14.88 | | | C |
| ANISOU | 357 | CG2 | ILE | A | 432 | 1643 | 1977 | 2033 | −828 | −209 | 417 | | C |
| ATOM | 358 | C | ILE | A | 432 | −5.446 | −9.512 | 1.588 | 1.00 | 21.32 | | | C |
| ANISOU | 358 | C | ILE | A | 432 | 2571 | 2551 | 2977 | −900 | −210 | 622 | | C |
| ATOM | 359 | O | ILE | A | 432 | −4.683 | −9.401 | 0.624 | 1.00 | 26.70 | | | O |
| ANISOU | 359 | O | ILE | A | 432 | 3178 | 3349 | 3620 | −972 | −187 | 722 | | O |
| ATOM | 360 | N | LYS | A | 433 | −5.805 | −8.473 | 2.339 | 1.00 | 25.08 | | | N |
| ANISOU | 360 | N | LYS | A | 433 | 3123 | 2840 | 3566 | −938 | −247 | 593 | | N |
| ATOM | 361 | CA | LYS | A | 433 | −5.310 | −7.141 | 2.014 | 1.00 | 33.77 | | | C |
| ANISOU | 361 | CA | LYS | A | 433 | 4230 | 3851 | 4749 | −1040 | −255 | 663 | | C |
| ATOM | 362 | CB | LYS | A | 433 | −5.933 | −6.080 | 2.922 | 1.00 | 41.11 | | | C |
| ANISOU | 362 | CB | LYS | A | 433 | 5261 | 4563 | 5796 | −1026 | −282 | 585 | | C |
| ATOM | 363 | CG | LYS | A | 433 | −5.749 | −6.334 | 4.394 | 1.00 | 40.86 | | | C |
| ANISOU | 363 | CG | LYS | A | 433 | 5244 | 4502 | 5777 | −1027 | −317 | 427 | | C |
| ATOM | 364 | CD | LYS | A | 433 | −5.455 | −5.044 | 5.145 | 1.00 | 47.52 | | | C |
| ANISOU | 364 | CD | LYS | A | 433 | 6135 | 5199 | 6723 | −1088 | −334 | 351 | | C |
| ATOM | 365 | CE | LYS | A | 433 | −6.592 | −4.046 | 5.032 | 1.00 | 46.11 | | | C |
| ANISOU | 365 | CE | LYS | A | 433 | 6058 | 4828 | 6633 | −1023 | −324 | 365 | | C |
| ATOM | 366 | NZ | LYS | A | 433 | −6.278 | −2.803 | 5.791 | 1.00 | 49.72 | | | N |
| ANISOU | 366 | NZ | LYS | A | 433 | 6565 | 5137 | 7188 | −1089 | −333 | 277 | | N |
| ATOM | 367 | C | LYS | A | 433 | −3.797 | −7.105 | 2.134 | 1.00 | 29.90 | | | C |
| ANISOU | 367 | C | LYS | A | 433 | 3647 | 3481 | 4233 | −1147 | −255 | 653 | | C |
| ATOM | 368 | O | LYS | A | 433 | −3.214 | −7.699 | 3.050 | 1.00 | 21.53 | | | O |
| ANISOU | 368 | O | LYS | A | 433 | 2545 | 2492 | 3144 | −1162 | −274 | 555 | | O |
| ATOM | 369 | N | GLU | A | 434 | −3.166 | −6.402 | 1.202 | 1.00 | 27.08 | | | N |
| ANISOU | 369 | N | GLU | A | 434 | 3251 | 3157 | 3881 | −1217 | −234 | 758 | | N |
| ATOM | 370 | CA | GLU | A | 434 | −1.716 | −6.407 | 1.120 | 1.00 | 30.32 | | | C |
| ANISOU | 370 | CA | GLU | A | 434 | 3557 | 3710 | 4251 | −1313 | −227 | 767 | | C |
| ATOM | 371 | CB | GLU | A | 434 | −1.242 | −5.718 | −0.159 | 1.00 | 36.52 | | | C |
| ANISOU | 371 | CB | GLU | A | 434 | 4305 | 4544 | 5027 | −1373 | −199 | 908 | | C |
| ATOM | 372 | CG | GLU | A | 434 | −1.417 | −4.218 | −0.161 | 1.00 | 40.45 | | | C |
| ANISOU | 372 | CG | GLU | A | 434 | 4880 | 4838 | 5652 | −1440 | −210 | 949 | | C |
| ATOM | 373 | CD | GLU | A | 434 | −2.417 | −3.761 | −1.204 | 0.00 | 37.12 | | | C |
| ANISOU | 373 | CD | GLU | A | 434 | 4516 | 4344 | 5244 | −1384 | −194 | 1069 | | C |
| ATOM | 374 | OE1 | GLU | A | 434 | −3.561 | −4.260 | −1.189 | 0.00 | 34.41 | | | O |
| ANISOU | 374 | OE1 | GLU | A | 434 | 4225 | 3965 | 4884 | −1273 | −196 | 1051 | | O |
| ATOM | 375 | OE2 | GLU | A | 434 | −2.054 | −2.914 | −2.047 | 0.00 | 37.35 | | | O |
| ANISOU | 375 | OE2 | GLU | A | 434 | 4532 | 4362 | 5296 | −1450 | −180 | 1185 | | O |
| ATOM | 376 | C | GLU | A | 434 | −1.114 | −5.726 | 2.337 | 1.00 | 26.81 | | | C |
| ANISOU | 376 | C | GLU | A | 434 | 3126 | 3173 | 3887 | −1392 | −264 | 659 | | C |
| ATOM | 377 | O | GLU | A | 434 | −1.681 | −4.783 | 2.897 | 1.00 | 29.14 | | | O |
| ANISOU | 377 | O | GLU | A | 434 | 3515 | 3267 | 4289 | −1401 | −283 | 612 | | O |
| ATOM | 378 | N | GLY | A | 435 | 0.041 | −6.232 | 2.759 | 1.00 | 22.92 | | | N |
| ANISOU | 378 | N | GLY | A | 435 | 2532 | 2842 | 3335 | −1439 | −270 | 609 | | N |
| ATOM | 379 | CA | GLY | A | 435 | 0.694 | −5.725 | 3.945 | 1.00 | 22.74 | | | C |
| ANISOU | 379 | CA | GLY | A | 435 | 2501 | 2774 | 3365 | −1512 | −308 | 494 | | C |
| ATOM | 380 | C | GLY | A | 435 | 0.147 | −6.247 | 5.255 | 1.00 | 26.22 | | | C |
| ANISOU | 380 | C | GLY | A | 435 | 2985 | 3174 | 3804 | −1445 | −346 | 351 | | C |
| ATOM | 381 | O | GLY | A | 435 | 0.705 | −5.910 | 6.307 | 1.00 | 25.39 | | | O |
| ANISOU | 381 | O | GLY | A | 435 | 2863 | 3060 | 3723 | −1497 | −382 | 241 | | O |
| ATOM | 382 | N | SER | A | 436 | −0.915 | −7.059 | 5.232 | 1.00 | 23.16 | | | N |
| ANISOU | 382 | N | SER | A | 436 | 2648 | 2770 | 3381 | −1334 | −341 | 346 | | N |
| ATOM | 383 | CA | SER | A | 436 | −1.541 | −7.530 | 6.462 | 1.00 | 26.78 | | | C |
| ANISOU | 383 | CA | SER | A | 436 | 3158 | 3184 | 3833 | −1267 | −378 | 216 | | C |
| ATOM | 384 | CB | SER | A | 436 | −3.063 | −7.648 | 6.293 | 1.00 | 20.95 | | | C |
| ANISOU | 384 | CB | SER | A | 436 | 2531 | 2305 | 3125 | −1162 | −370 | 228 | | C |
| ATOM | 385 | OG | SER | A | 436 | −3.443 | −8.729 | 5.445 | 1.00 | 26.28 | | | O |
| ANISOU | 385 | OG | SER | A | 436 | 3181 | 3095 | 3710 | −1052 | −323 | 294 | | O |
| ATOM | 386 | C | SER | A | 436 | −1.000 | −8.868 | 6.951 | 1.00 | 31.39 | | | C |
| ANISOU | 386 | C | SER | A | 436 | 3655 | 3975 | 4296 | −1198 | −378 | 164 | | C |
| ATOM | 387 | O | SER | A | 436 | −0.974 | −9.100 | 8.166 | 1.00 | 24.49 | | | O |
| ANISOU | 387 | O | SER | A | 436 | 2789 | 3115 | 3400 | −1161 | −411 | 45 | | O |
| ATOM | 388 | N | MET | A | 437 | −0.555 | −9.749 | 6.051 | 1.00 | 17.72 | | | N |
| ANISOU | 388 | N | MET | A | 437 | 1842 | 2409 | 2481 | −1152 | −335 | 242 | | N |
| ATOM | 389 | CA | MET | A | 437 | −0.228 | −11.123 | 6.414 | 1.00 | 25.42 | | | C |
| ANISOU | 389 | CA | MET | A | 437 | 2758 | 3552 | 3349 | −1027 | −319 | 194 | | C |
| ATOM | 390 | CB | MET | A | 437 | −1.235 | −12.084 | 5.753 | 1.00 | 21.69 | | | C |
| ANISOU | 390 | CB | MET | A | 437 | 2342 | 3067 | 2835 | −882 | −273 | 222 | | C |
| ATOM | 391 | CG | MET | A | 437 | −0.843 | −13.554 | 5.724 | 1.00 | 37.01 | | | C |
| ANISOU | 391 | CG | MET | A | 437 | 4220 | 5166 | 4675 | −756 | −243 | 202 | | C |
| ATOM | 392 | SD | MET | A | 437 | −2.139 | −14.614 | 5.006 | 1.00 | 46.06 | | | S |
| ANISOU | 392 | SD | MET | A | 437 | 5447 | 6264 | 5789 | −610 | −196 | 213 | | S |
| ATOM | 393 | CE | MET | A | 437 | −3.370 | −14.587 | 6.311 | 1.00 | 46.04 | | | C |
| ANISOU | 393 | CE | MET | A | 437 | 5562 | 6102 | 5830 | −549 | −227 | 130 | | C |
| ATOM | 394 | C | MET | A | 437 | 1.205 | −11.482 | 6.023 | 1.00 | 30.15 | | | C |
| ANISOU | 394 | C | MET | A | 437 | 3207 | 4368 | 3880 | −1076 | −306 | 234 | | C |
| ATOM | 395 | O | MET | A | 437 | 1.761 | −10.939 | 5.064 | 1.00 | 18.58 | | | O |
| ANISOU | 395 | O | MET | A | 437 | 1682 | 2951 | 2426 | −1181 | −288 | 328 | | O |
| ATOM | 396 | N | SER | A | 438 | 1.812 | −12.386 | 6.793 | 1.00 | 17.50 | | | N |
| ANISOU | 396 | N | SER | A | 438 | 1538 | 2907 | 2204 | −996 | −317 | 170 | | N |
| ATOM | 397 | CA | SER | A | 438 | 3.107 | −12.981 | 6.436 | 1.00 | 19.84 | | | C |
| ANISOU | 397 | CA | SER | A | 438 | 1683 | 3434 | 2420 | −993 | −298 | 203 | | C |
| ATOM | 398 | CB | SER | A | 438 | 3.776 | −13.559 | 7.678 | 1.00 | 20.51 | | | C |
| ANISOU | 398 | CB | SER | A | 438 | 1703 | 3639 | 2450 | −937 | −336 | 123 | | C |
| ATOM | 399 | OG | SER | A | 438 | 5.121 | −13.935 | 7.455 | 1.00 | 19.42 | | | O |
| ANISOU | 399 | OG | SER | A | 438 | 1412 | 3722 | 2243 | −940 | −325 | 151 | | O |
| ATOM | 400 | C | SER | A | 438 | 2.848 | −14.055 | 5.374 | 1.00 | 20.16 | | | C |
| ANISOU | 400 | C | SER | A | 438 | 1717 | 3543 | 2400 | −861 | −234 | 252 | | C |
| ATOM | 401 | O | SER | A | 438 | 2.573 | −15.214 | 5.684 | 1.00 | 16.97 | | | O |
| ANISOU | 401 | O | SER | A | 438 | 1334 | 3166 | 1949 | −706 | −218 | 209 | | O |
| ATOM | 402 | N | GLU | A | 439 | 2.938 | −13.673 | 4.090 | 1.00 | 21.33 | | | N |
| ANISOU | 402 | N | GLU | A | 439 | 1836 | 3722 | 2547 | −925 | −196 | 343 | | N |
| ATOM | 403 | CA | GLU | A | 439 | 2.280 | −14.477 | 3.055 | 1.00 | 22.10 | | | C |
| ANISOU | 403 | CA | GLU | A | 439 | 1969 | 3828 | 2599 | −813 | −141 | 372 | | C |
| ATOM | 404 | CB | GLU | A | 439 | 2.075 | −13.641 | 1.791 | 1.00 | 26.95 | | | C |
| ANISOU | 404 | CB | GLU | A | 439 | 2583 | 4426 | 3230 | −917 | −114 | 481 | | C |
| ATOM | 405 | CG | GLU | A | 439 | 1.066 | −12.514 | 1.967 | 1.00 | 28.48 | | | C |
| ANISOU | 405 | CG | GLU | A | 439 | 2899 | 4392 | 3528 | −998 | −147 | 509 | | C |
| ATOM | 406 | CD | GLU | A | 439 | 0.818 | −11.707 | 0.686 | 1.00 | 36.99 | | | C |
| ANISOU | 406 | CD | GLU | A | 439 | 3980 | 5453 | 4623 | −1089 | −122 | 641 | | C |
| ATOM | 407 | OE1 | GLU | A | 439 | 1.388 | −12.055 | −0.373 | 1.00 | 43.12 | | | O |
| ANISOU | 407 | OE1 | GLU | A | 439 | 4675 | 6396 | 5314 | −1074 | −78 | 700 | | O |
| ATOM | 408 | OE2 | GLU | A | 439 | 0.048 | −10.722 | 0.748 | 1.00 | 31.73 | | | O |
| ANISOU | 408 | OE2 | GLU | A | 439 | 3412 | 4596 | 4050 | −1146 | −148 | 678 | | O |
| ATOM | 409 | C | GLU | A | 439 | 3.048 | −15.762 | 2.744 | 1.00 | 17.18 | | | C |
| ANISOU | 409 | C | GLU | A | 439 | 1246 | 3401 | 1879 | −686 | −100 | 349 | | C |
| ATOM | 410 | O | GLU | A | 439 | 2.439 | −16.831 | 2.623 | 1.00 | 19.20 | | | O |
| ANISOU | 410 | O | GLU | A | 439 | 1558 | 3630 | 2106 | −540 | −74 | 306 | | O |
| ATOM | 411 | N | ASP | A | 440 | 4.373 | −15.687 | 2.616 | 1.00 | 18.39 | | | N |
| ANISOU | 411 | N | ASP | A | 440 | 1251 | 3750 | 1987 | −736 | −95 | 375 | | N |
| ATOM | 412 | CA | ASP | A | 440 | 5.151 | −16.900 | 2.357 | 1.00 | 24.90 | | | C |
| ANISOU | 412 | CA | ASP | A | 440 | 1974 | 4765 | 2723 | −596 | −55 | 347 | | C |
| ATOM | 413 | CB | ASP | A | 440 | 6.628 | −16.564 | 2.175 | 1.00 | 20.28 | | | C |
| ANISOU | 413 | CB | ASP | A | 440 | 1269 | 4350 | 2087 | −653 | −51 | 379 | | C |
| ATOM | 414 | CG | ASP | A | 440 | 6.913 | −15.908 | 0.845 | 1.00 | 31.95 | | | C |
| ANISOU | 414 | CG | ASP | A | 440 | 2728 | 5876 | 3535 | −742 | −15 | 462 | | C |
| ATOM | 415 | OD1 | ASP | A | 440 | 6.085 | −16.082 | −0.077 | 1.00 | 32.37 | | | O |
| ANISOU | 415 | OD1 | ASP | A | 440 | 2830 | 5890 | 3577 | −714 | 20 | 489 | | O |
| ATOM | 416 | OD2 | ASP | A | 440 | 7.957 | −15.224 | 0.726 | 1.00 | 36.84 | | | O |
| ANISOU | 416 | OD2 | ASP | A | 440 | 3282 | 6578 | 4138 | −838 | −24 | 503 | | O |
| ATOM | 417 | C | ASP | A | 440 | 4.983 | −17.929 | 3.473 | 1.00 | 26.56 | | | C |
| ANISOU | 417 | C | ASP | A | 440 | 2227 | 4934 | 2929 | −442 | −77 | 264 | | C |
| ATOM | 418 | O | ASP | A | 440 | 4.818 | −19.124 | 3.203 | 1.00 | 19.14 | | | O |
| ANISOU | 418 | O | ASP | A | 440 | 1305 | 4015 | 1952 | −281 | −39 | 229 | | O |
| ATOM | 419 | N | GLU | A | 441 | 5.044 | −17.497 | 4.735 | 1.00 | 17.99 | | | N |
| ANISOU | 419 | N | GLU | A | 441 | 1159 | 3793 | 1882 | −489 | −137 | 232 | | N |
| ATOM | 420 | CA | GLU | A | 441 | 4.786 | −18.436 | 5.826 | 1.00 | 17.39 | | | C |
| ANISOU | 420 | CA | GLU | A | 441 | 1132 | 3678 | 1797 | −346 | −158 | 173 | | C |
| ATOM | 421 | CB | GLU | A | 441 | 5.035 | −17.768 | 7.172 | 1.00 | 18.42 | | | C |
| ANISOU | 421 | CB | GLU | A | 441 | 1252 | 3800 | 1949 | −427 | −228 | 139 | | C |
| ATOM | 422 | CG | GLU | A | 441 | 6.468 | −17.394 | 7.445 | 1.00 | 22.58 | | | C |
| ANISOU | 422 | CG | GLU | A | 441 | 1616 | 4531 | 2434 | −503 | −254 | 150 | | C |
| ATOM | 423 | CD | GLU | A | 441 | 6.572 | −16.653 | 8.765 | 1.00 | 28.03 | | | C |
| ANISOU | 423 | CD | GLU | A | 441 | 2329 | 5175 | 3144 | −589 | −321 | 98 | | C |
| ATOM | 424 | OE1 | GLU | A | 441 | 7.237 | −15.597 | 8.822 | 1.00 | 27.61 | | | O |
| ANISOU | 424 | OE1 | GLU | A | 441 | 2247 | 5129 | 3113 | −737 | −339 | 99 | | O |
| ATOM | 425 | OE2 | GLU | A | 441 | 5.957 | −17.128 | 9.743 | 1.00 | 28.12 | | | O |
| ANISOU | 425 | OE2 | GLU | A | 441 | 2395 | 5141 | 3149 | −507 | −354 | 53 | | O |
| ATOM | 426 | C | GLU | A | 441 | 3.362 | −18.976 | 5.781 | 1.00 | 16.02 | | | C |
| ANISOU | 426 | C | GLU | A | 441 | 1118 | 3310 | 1658 | −259 | −140 | 148 | | C |
| ATOM | 427 | O | GLU | A | 441 | 3.129 | −20.161 | 6.054 | 1.00 | 16.09 | | | O |
| ANISOU | 427 | O | GLU | A | 441 | 1163 | 3303 | 1649 | −106 | −123 | 119 | | O |
| ATOM | 428 | N | PHE | A | 442 | 2.379 | −18.118 | 5.501 | 1.00 | 15.41 | | | N |
| ANISOU | 428 | N | PHE | A | 442 | 1140 | 3078 | 1638 | −354 | −147 | 161 | | N |
| ATOM | 429 | CA | PHE | A | 442 | 1.015 | −18.629 | 5.464 | 1.00 | 20.21 | | | C |
| ANISOU | 429 | CA | PHE | A | 442 | 1883 | 3523 | 2272 | −275 | −132 | 137 | | C |
| ATOM | 430 | CB | PHE | A | 442 | 0.002 | −17.509 | 5.236 | 1.00 | 21.50 | | | C |
| ANISOU | 430 | CB | PHE | A | 442 | 2137 | 3531 | 2499 | −383 | −147 | 158 | | C |
| ATOM | 431 | CG | PHE | A | 442 | −1.330 | −18.021 | 4.773 | 1.00 | 24.90 | | | C |
| ANISOU | 431 | CG | PHE | A | 442 | 2676 | 3841 | 2942 | −312 | −120 | 150 | | C |
| ATOM | 432 | CD1 | PHE | A | 442 | −2.168 | −18.703 | 5.651 | 1.00 | 26.06 | | | C |
| ANISOU | 432 | CD1 | PHE | A | 442 | 2907 | 3894 | 3102 | −225 | −129 | 102 | | C |
| ATOM | 433 | CE1 | PHE | A | 442 | −3.392 | −19.200 | 5.222 | 1.00 | 23.69 | | | C |
| ANISOU | 433 | CE1 | PHE | A | 442 | 2695 | 3495 | 2811 | −173 | −105 | 94 | | C |
| ATOM | 434 | CZ | PHE | A | 442 | −3.786 | −19.024 | 3.888 | 1.00 | 23.59 | | | C |
| ANISOU | 434 | CZ | PHE | A | 442 | 2687 | 3487 | 2790 | −201 | −76 | 129 | | C |
| ATOM | 435 | CE2 | PHE | A | 442 | −2.950 | −18.351 | 3.003 | 1.00 | 24.84 | | | C |
| ANISOU | 435 | CE2 | PHE | A | 442 | 2764 | 3747 | 2928 | −279 | −65 | 183 | | C |
| ATOM | 436 | CD2 | PHE | A | 442 | −1.725 | −17.863 | 3.449 | 1.00 | 25.64 | | | C |
| ANISOU | 436 | CD2 | PHE | A | 442 | 2777 | 3938 | 3026 | −337 | −85 | 196 | | C |
| ATOM | 437 | C | PHE | A | 442 | 0.864 | −19.691 | 4.383 | 1.00 | 21.94 | | | C |
| ANISOU | 437 | C | PHE | A | 442 | 2102 | 3782 | 2451 | −168 | −74 | 138 | | C |
| ATOM | 438 | O | PHE | A | 442 | 0.228 | −20.729 | 4.602 | 1.00 | 16.88 | | | O |
| ANISOU | 438 | O | PHE | A | 442 | 1534 | 3069 | 1811 | −51 | −58 | 101 | | O |
| ATOM | 439 | N | ILE | A | 443 | 1.451 | −19.449 | 3.212 | 1.00 | 14.72 | | | N |
| ANISOU | 439 | N | ILE | A | 443 | 1108 | 2986 | 1500 | −213 | −39 | 178 | | N |
| ATOM | 440 | CA | ILE | A | 443 | 1.280 | −20.362 | 2.085 | 1.00 | 16.27 | | | C |
| ANISOU | 440 | CA | ILE | A | 443 | 1303 | 3231 | 1647 | −120 | 17 | 163 | | C |
| ATOM | 441 | CB | ILE | A | 443 | 1.901 | −19.745 | 0.824 | 1.00 | 22.66 | | | C |
| ANISOU | 441 | CB | ILE | A | 443 | 2014 | 4188 | 2406 | −206 | 50 | 223 | | C |
| ATOM | 442 | CG1 | ILE | A | 443 | 1.028 | −18.582 | 0.351 | 1.00 | 19.69 | | | C |
| ANISOU | 442 | CG1 | ILE | A | 443 | 1704 | 3705 | 2071 | −337 | 35 | 288 | | C |
| ATOM | 443 | CD1 | ILE | A | 443 | 1.650 | −17.773 | −0.781 | 1.00 | 22.37 | | | C |
| ANISOU | 443 | CD1 | ILE | A | 443 | 1947 | 4184 | 2368 | −449 | 61 | 379 | | C |
| ATOM | 444 | CG2 | ILE | A | 443 | 2.090 | −20.810 | −0.259 | 1.00 | 28.28 | | | C |
| ANISOU | 444 | CG2 | ILE | A | 443 | 2688 | 5015 | 3043 | −93 | 111 | 184 | | C |
| ATOM | 445 | C | ILE | A | 443 | 1.883 | −21.721 | 2.396 | 1.00 | 15.49 | | | C |
| ANISOU | 445 | C | ILE | A | 443 | 1168 | 3204 | 1515 | 41 | 38 | 108 | | C |
| ATOM | 446 | O | ILE | A | 443 | 1.310 | −22.764 | 2.063 | 1.00 | 18.33 | | | O |
| ANISOU | 446 | O | ILE | A | 443 | 1593 | 3501 | 1871 | 151 | 69 | 59 | | O |
| ATOM | 447 | N | GLU | A | 444 | 3.045 | −21.734 | 3.041 | 1.00 | 15.86 | | | N |
| ANISOU | 447 | N | GLU | A | 444 | 1108 | 3378 | 1541 | 57 | 20 | 116 | | N |
| ATOM | 448 | CA | GLU | A | 444 | 3.622 | −23.005 | 3.447 | 1.00 | 16.32 | | | C |
| ANISOU | 448 | CA | GLU | A | 444 | 1132 | 3494 | 1576 | 227 | 35 | 77 | | C |
| ATOM | 449 | CB | GLU | A | 444 | 5.011 | −22.769 | 4.045 | 1.00 | 19.70 | | | C |
| ANISOU | 449 | CB | GLU | A | 444 | 1410 | 4108 | 1968 | 220 | 11 | 102 | | C |
| ATOM | 450 | CG | GLU | A | 444 | 5.806 | −24.039 | 4.281 | 1.00 | 28.87 | | | C |
| ANISOU | 450 | CG | GLU | A | 444 | 2505 | 5369 | 3096 | 411 | 32 | 77 | | C |
| ATOM | 451 | CD | GLU | A | 444 | 6.038 | −24.833 | 3.001 | 1.00 | 33.62 | | | C |
| ANISOU | 451 | CD | GLU | A | 444 | 3074 | 6043 | 3659 | 517 | 102 | 40 | | C |
| ATOM | 452 | OE1 | GLU | A | 444 | 6.161 | −24.210 | 1.915 | 1.00 | 28.52 | | | O |
| ANISOU | 452 | OE1 | GLU | A | 444 | 2376 | 5486 | 2974 | 421 | 133 | 54 | | O |
| ATOM | 453 | OE2 | GLU | A | 444 | 6.090 | −26.082 | 3.091 | 1.00 | 34.80 | | | O |
| ANISOU | 453 | OE2 | GLU | A | 444 | 3252 | 6155 | 3814 | 696 | 128 | −3 | | O |
| ATOM | 454 | C | GLU | A | 444 | 2.702 | −23.725 | 4.427 | 1.00 | 18.19 | | | C |
| ANISOU | 454 | C | GLU | A | 444 | 1497 | 3551 | 1861 | 312 | 14 | 49 | | C |
| ATOM | 455 | O | GLU | A | 444 | 2.496 | −24.940 | 4.319 | 1.00 | 18.05 | | | O |
| ANISOU | 455 | O | GLU | A | 444 | 1526 | 3482 | 1849 | 453 | 44 | 12 | | O |
| ATOM | 456 | N | GLU | A | 445 | 2.103 | −22.983 | 5.365 | 1.00 | 14.77 | | | N |
| ANISOU | 456 | N | GLU | A | 445 | 1127 | 3018 | 1468 | 223 | −35 | 64 | | N |
| ATOM | 457 | CA | GLU | A | 445 | 1.175 | −23.597 | 6.309 | 1.00 | 14.79 | | | C |
| ANISOU | 457 | CA | GLU | A | 445 | 1244 | 2868 | 1507 | 291 | −52 | 47 | | C |
| ATOM | 458 | CB | GLU | A | 445 | 0.866 | −22.643 | 7.469 | 1.00 | 18.67 | | | C |
| ANISOU | 458 | CB | GLU | A | 445 | 1761 | 3315 | 2019 | 194 | −109 | 57 | | C |
| ATOM | 459 | CG | GLU | A | 445 | −0.045 | −23.239 | 8.555 | 1.00 | 17.39 | | | C |
| ANISOU | 459 | CG | GLU | A | 445 | 1701 | 3030 | 1878 | 260 | −126 | 49 | | C |
| ATOM | 460 | CD | GLU | A | 445 | 0.662 | −24.292 | 9.404 | 1.00 | 24.08 | | | C |
| ANISOU | 460 | CD | GLU | A | 445 | 2506 | 3948 | 2695 | 396 | −134 | 67 | | C |
| ATOM | 461 | OE1 | GLU | A | 445 | 1.867 | −24.537 | 9.176 | 1.00 | 26.24 | | | O |
| ANISOU | 461 | OE1 | GLU | A | 445 | 2666 | 4370 | 2933 | 446 | −129 | 78 | | O |
| ATOM | 462 | OE2 | GLU | A | 445 | 0.013 | −24.876 | 10.299 | 1.00 | 30.89 | | | O |
| ANISOU | 462 | OE2 | GLU | A | 445 | 3443 | 4727 | 3568 | 455 | −144 | 80 | | O |
| ATOM | 463 | C | GLU | A | 445 | −0.116 | −24.021 | 5.624 | 1.00 | 16.82 | | | C |
| ANISOU | 463 | C | GLU | A | 445 | 1621 | 2974 | 1796 | 305 | −20 | 23 | | C |
| ATOM | 464 | O | GLU | A | 445 | −0.724 | −25.017 | 6.029 | 1.00 | 18.59 | | | O |
| ANISOU | 464 | O | GLU | A | 445 | 1925 | 3093 | 2043 | 398 | −11 | 6 | | O |
| ATOM | 465 | N | ALA | A | 446 | −0.551 | −23.291 | 4.589 | 1.00 | 12.91 | | | N |
| ANISOU | 465 | N | ALA | A | 446 | 1136 | 2470 | 1301 | 209 | −4 | 28 | | N |
| ATOM | 466 | CA | ALA | A | 446 | −1.747 | −23.710 | 3.868 | 1.00 | 12.28 | | | C |
| ANISOU | 466 | CA | ALA | A | 446 | 1153 | 2276 | 1237 | 222 | 23 | 2 | | C |
| ATOM | 467 | CB | ALA | A | 446 | −2.070 | −22.719 | 2.741 | 1.00 | 13.01 | | | C |
| ANISOU | 467 | CB | ALA | A | 446 | 1231 | 2396 | 1314 | 110 | 32 | 29 | | C |
| ATOM | 468 | C | ALA | A | 446 | −1.597 | −25.127 | 3.325 | 1.00 | 12.74 | | | C |
| ANISOU | 468 | C | ALA | A | 446 | 1221 | 2340 | 1280 | 353 | 67 | −50 | | C |
| ATOM | 469 | O | ALA | A | 446 | −2.584 | −25.863 | 3.235 | 1.00 | 20.13 | | | O |
| ANISOU | 469 | O | ALA | A | 446 | 2253 | 3152 | 2244 | 391 | 81 | −85 | | O |
| ATOM | 470 | N | LYS | A | 447 | −0.371 | −25.535 | 2.976 | 1.00 | 13.74 | | | N |
| ANISOU | 470 | N | LYS | A | 447 | 1247 | 2608 | 1365 | 425 | 91 | −60 | | N |
| ATOM | 471 | CA | LYS | A | 447 | −0.153 | −26.889 | 2.471 | 1.00 | 22.10 | | | C |
| ANISOU | 471 | CA | LYS | A | 447 | 2317 | 3665 | 2417 | 567 | 135 | −123 | | C |
| ATOM | 472 | CB | LYS | A | 447 | 1.293 | −27.054 | 1.999 | 1.00 | 19.71 | | | C |
| ANISOU | 472 | CB | LYS | A | 447 | 1875 | 3558 | 2056 | 636 | 162 | −129 | | C |
| ATOM | 473 | CG | LYS | A | 447 | 1.700 | −26.121 | 0.879 | 1.00 | 19.03 | | | C |
| ANISOU | 473 | CG | LYS | A | 447 | 1696 | 3631 | 1905 | 532 | 182 | −111 | | C |
| ATOM | 474 | CD | LYS | A | 447 | 3.186 | −26.293 | 0.580 | 1.00 | 23.56 | | | C |
| ANISOU | 474 | CD | LYS | A | 447 | 2114 | 4421 | 2417 | 601 | 209 | −110 | | C |
| ATOM | 475 | CE | LYS | A | 447 | 3.626 | −25.446 | −0.595 | 1.00 | 24.10 | | | C |
| ANISOU | 475 | CE | LYS | A | 447 | 2080 | 4666 | 2410 | 498 | 237 | −81 | | C |
| ATOM | 476 | NZ | LYS | A | 447 | 5.048 | −25.757 | −0.991 | 1.00 | 29.09 | | | N |
| ANISOU | 476 | NZ | LYS | A | 447 | 2558 | 5522 | 2973 | 576 | 271 | −90 | | N |
| ATOM | 477 | C | LYS | A | 447 | −0.479 | −27.936 | 3.526 | 1.00 | 22.77 | | | C |
| ANISOU | 477 | C | LYS | A | 447 | 2484 | 3611 | 2558 | 673 | 124 | −129 | | C |
| ATOM | 478 | O | LYS | A | 447 | −0.984 | −29.016 | 3.203 | 1.00 | 25.57 | | | O |
| ANISOU | 478 | O | LYS | A | 447 | 2916 | 3857 | 2943 | 754 | 153 | −184 | | O |
| ATOM | 479 | N | VAL | A | 448 | −0.172 | −27.642 | 4.789 | 1.00 | 22.23 | | | N |
| ANISOU | 479 | N | VAL | A | 448 | 2396 | 3547 | 2501 | 671 | 82 | −73 | | N |
| ATOM | 480 | CA | VAL | A | 448 | −0.518 | −28.545 | 5.883 | 1.00 | 24.99 | | | C |
| ANISOU | 480 | CA | VAL | A | 448 | 2821 | 3778 | 2896 | 762 | 68 | −53 | | C |
| ATOM | 481 | CB | VAL | A | 448 | 0.287 | −28.187 | 7.147 | 1.00 | 24.88 | | | C |
| ANISOU | 481 | CB | VAL | A | 448 | 2734 | 3860 | 2860 | 777 | 22 | 9 | | C |
| ATOM | 482 | CG1 | VAL | A | 448 | −0.120 | −29.103 | 8.306 | 1.00 | 32.78 | | | C |
| ANISOU | 482 | CG1 | VAL | A | 448 | 3810 | 4749 | 3895 | 869 | 8 | 50 | | C |
| ATOM | 483 | CG2 | VAL | A | 448 | 1.779 | −28.272 | 6.877 | 1.00 | 24.87 | | | C |
| ANISOU | 483 | CG2 | VAL | A | 448 | 2590 | 4047 | 2811 | 850 | 30 | 12 | | C |
| ATOM | 484 | C | VAL | A | 448 | −2.011 | −28.499 | 6.158 | 1.00 | 19.07 | | | C |
| ANISOU | 484 | C | VAL | A | 448 | 2197 | 2859 | 2190 | 690 | 59 | −54 | | C |
| ATOM | 485 | O | VAL | A | 448 | −2.647 | −29.534 | 6.384 | 1.00 | 19.15 | | | O |
| ANISOU | 485 | O | VAL | A | 448 | 2297 | 2733 | 2245 | 756 | 74 | −63 | | O |
| ATOM | 486 | N | MET | A | 449 | −2.592 | −27.301 | 6.160 | 1.00 | 12.39 | | | N |
| ANISOU | 486 | N | MET | A | 449 | 1357 | 2017 | 1335 | 556 | 34 | −41 | | N |
| ATOM | 487 | CA | MET | A | 449 | −4.014 | −27.173 | 6.442 | 1.00 | 15.04 | | | C |
| ANISOU | 487 | CA | MET | A | 449 | 1796 | 2215 | 1705 | 493 | 26 | −40 | | C |
| ATOM | 488 | CB | MET | A | 449 | −4.384 | −25.695 | 6.541 | 1.00 | 15.17 | | | C |
| ANISOU | 488 | CB | MET | A | 449 | 1797 | 2256 | 1712 | 364 | −5 | −22 | | C |
| ATOM | 489 | CG | MET | A | 449 | −3.575 | −24.964 | 7.605 | 1.00 | 18.17 | | | C |
| ANISOU | 489 | CG | MET | A | 449 | 2112 | 2719 | 2071 | 341 | −47 | 10 | | C |
| ATOM | 490 | SD | MET | A | 449 | −3.764 | −23.187 | 7.481 | 1.00 | 21.82 | | | S |
| ANISOU | 490 | SD | MET | A | 449 | 2552 | 3199 | 2537 | 189 | −80 | 16 | | S |
| ATOM | 491 | CE | MET | A | 449 | −5.497 | −23.009 | 7.865 | 1.00 | 20.19 | | | C |
| ANISOU | 491 | CE | MET | A | 449 | 2462 | 2841 | 2369 | 157 | −84 | 7 | | C |
| ATOM | 492 | C | MET | A | 449 | −4.893 | −27.883 | 5.415 | 1.00 | 17.58 | | | C |
| ANISOU | 492 | C | MET | A | 449 | 2188 | 2445 | 2048 | 499 | 63 | −92 | | C |
| ATOM | 493 | O | MET | A | 449 | −6.020 | −28.259 | 5.748 | 1.00 | 19.05 | | | O |
| ANISOU | 493 | O | MET | A | 449 | 2460 | 2510 | 2270 | 480 | 62 | −93 | | O |
| ATOM | 494 | N | AMET | A | 450 | −4.404 | −28.077 | 4.185 | 0.86 | 19.38 | | | N |
| ANISOU | 494 | N | AMET | A | 450 | 2374 | 2743 | 2248 | 519 | 95 | −140 | | N |
| ATOM | 495 | CA | AMET | A | 450 | −5.189 | −28.800 | 3.185 | 0.86 | 18.93 | | | C |
| ANISOU | 495 | CA | AMET | A | 450 | 2378 | 2615 | 2200 | 524 | 127 | −209 | | C |
| ATOM | 496 | CB | AMET | A | 450 | −4.420 | −28.895 | 1.865 | 0.86 | 19.01 | | | C |
| ANISOU | 496 | CB | AMET | A | 450 | 2318 | 2751 | 2155 | 553 | 163 | −266 | | C |
| ATOM | 497 | CG | AMET | A | 450 | −4.259 | −27.566 | 1.145 | 0.86 | 18.56 | | | C |
| ANISOU | 497 | CG | AMET | A | 450 | 2186 | 2826 | 2039 | 447 | 155 | −230 | | C |
| ATOM | 498 | SD | AMET | A | 450 | −5.836 | −26.994 | 0.468 | 0.86 | 17.62 | | | S |
| ANISOU | 498 | SD | AMET | A | 450 | 2134 | 2644 | 1917 | 331 | 144 | −232 | | S |
| ATOM | 499 | CE | AMET | A | 450 | −6.289 | −28.389 | −0.554 | 0.86 | 19.03 | | | C |
| ANISOU | 499 | CE | AMET | A | 450 | 2362 | 2786 | 2083 | 393 | 186 | −353 | | C |
| ATOM | 500 | C | AMET | A | 450 | −5.557 | −30.196 | 3.664 | 0.86 | 19.14 | | | C |
| ANISOU | 500 | C | AMET | A | 450 | 2490 | 2496 | 2287 | 612 | 142 | −235 | | C |
| ATOM | 501 | O | AMET | A | 450 | −6.568 | −30.752 | 3.227 | 0.86 | 22.49 | | | O |
| ANISOU | 501 | O | AMET | A | 450 | 2991 | 2817 | 2738 | 583 | 156 | −284 | | O |
| ATOM | 502 | N | BMET | A | 450 | −4.411 | −28.089 | 4.188 | 0.14 | 18.90 | | | N |
| ANISOU | 502 | N | BMET | A | 450 | 2314 | 2681 | 2187 | 520 | 95 | −140 | | N |
| ATOM | 503 | CA | BMET | A | 450 | −5.233 | −28.786 | 3.202 | 0.14 | 18.82 | | | C |
| ANISOU | 503 | CA | BMET | A | 450 | 2366 | 2597 | 2187 | 521 | 127 | −208 | | C |
| ATOM | 504 | CB | BMET | A | 450 | −4.660 | −28.595 | 1.798 | 0.14 | 19.25 | | | C |
| ANISOU | 504 | CB | BMET | A | 450 | 2352 | 2783 | 2181 | 517 | 157 | −257 | | C |
| ATOM | 505 | CG | BMET | A | 450 | −4.878 | −27.201 | 1.239 | 0.14 | 18.46 | | | C |
| ANISOU | 505 | CG | BMET | A | 450 | 2205 | 2771 | 2038 | 394 | 141 | −213 | | C |
| ATOM | 506 | SD | BMET | A | 450 | −5.310 | −27.221 | −0.511 | 0.14 | 17.27 | | | S |
| ANISOU | 506 | SD | BMET | A | 450 | 2040 | 2701 | 1821 | 356 | 174 | −273 | | S |
| ATOM | 507 | CE | BMET | A | 450 | −6.802 | −28.211 | −0.489 | 0.14 | 17.57 | | | C |
| ANISOU | 507 | CE | BMET | A | 450 | 2201 | 2569 | 1907 | 358 | 176 | −342 | | C |
| ATOM | 508 | C | BMET | A | 450 | −5.389 | −30.272 | 3.497 | 0.14 | 19.71 | | | C |
| ANISOU | 508 | C | BMET | A | 450 | 2552 | 2582 | 2353 | 625 | 148 | −246 | | C |
| ATOM | 509 | O | BMET | A | 450 | −6.082 | −30.966 | 2.745 | 0.14 | 20.09 | | | O |
| ANISOU | 509 | O | BMET | A | 450 | 2661 | 2554 | 2418 | 621 | 172 | −317 | | O |
| ATOM | 510 | N | ASN | A | 451 | −4.757 | −30.775 | 4.554 | 1.00 | 18.33 | | | N |
| ANISOU | 510 | N | ASN | A | 451 | 2375 | 2385 | 2206 | 714 | 138 | −198 | | N |
| ATOM | 511 | CA | ASN | A | 451 | −4.974 | −32.125 | 5.046 | 1.00 | 21.02 | | | C |
| ANISOU | 511 | CA | ASN | A | 451 | 2797 | 2577 | 2614 | 808 | 153 | −201 | | C |
| ATOM | 512 | CB | ASN | A | 451 | −3.645 | −32.766 | 5.434 | 1.00 | 21.37 | | | C |
| ANISOU | 512 | CB | ASN | A | 451 | 2786 | 2671 | 2662 | 959 | 161 | −179 | | C |
| ATOM | 513 | CG | ASN | A | 451 | −2.649 | −32.763 | 4.299 | 1.00 | 40.60 | | | C |
| ANISOU | 513 | CG | ASN | A | 451 | 5136 | 5239 | 5051 | 1017 | 192 | −253 | | C |
| ATOM | 514 | OD1 | ASN | A | 451 | −3.008 | −32.978 | 3.144 | 1.00 | 42.04 | | | O |
| ANISOU | 514 | OD1 | ASN | A | 451 | 5341 | 5409 | 5225 | 995 | 223 | −345 | | O |
| ATOM | 515 | ND2 | ASN | A | 451 | −1.386 | −32.511 | 4.623 | 1.00 | 49.26 | | | N |
| ANISOU | 515 | ND2 | ASN | A | 451 | 6124 | 6483 | 6109 | 1089 | 184 | −213 | | N |
| ATOM | 516 | C | ASN | A | 451 | −5.901 | −32.166 | 6.256 | 1.00 | 23.25 | | | C |
| ANISOU | 516 | C | ASN | A | 451 | 3149 | 2752 | 2934 | 763 | 128 | −126 | | C |
| ATOM | 517 | O | ASN | A | 451 | −6.210 | −33.257 | 6.743 | 1.00 | 23.52 | | | O |
| ANISOU | 517 | O | ASN | A | 451 | 3258 | 2649 | 3029 | 822 | 139 | −107 | | O |
| ATOM | 518 | N | LEU | A | 452 | −6.313 | −31.016 | 6.769 | 1.00 | 16.73 | | | N |
| ANISOU | 518 | N | LEU | A | 452 | 2299 | 1985 | 2074 | 665 | 96 | −82 | | N |
| ATOM | 519 | CA | LEU | A | 452 | −7.264 | −30.972 | 7.875 | 1.00 | 15.65 | | | C |
| ANISOU | 519 | CA | LEU | A | 452 | 2218 | 1771 | 1956 | 620 | 76 | −21 | | C |
| ATOM | 520 | CB | LEU | A | 452 | −7.110 | −29.678 | 8.662 | 1.00 | 18.18 | | | C |
| ANISOU | 520 | CB | LEU | A | 452 | 2482 | 2198 | 2228 | 561 | 37 | 22 | | C |
| ATOM | 521 | CG | LEU | A | 452 | −5.788 | −29.466 | 9.387 | 1.00 | 17.42 | | | C |
| ANISOU | 521 | CG | LEU | A | 452 | 2305 | 2218 | 2095 | 627 | 13 | 62 | | C |
| ATOM | 522 | CD1 | LEU | A | 452 | −5.790 | −28.077 | 10.006 | 1.00 | 19.48 | | | C |
| ANISOU | 522 | CD1 | LEU | A | 452 | 2519 | 2570 | 2313 | 539 | −27 | 75 | | C |
| ATOM | 523 | CD2 | LEU | A | 452 | −5.593 | −30.534 | 10.452 | 1.00 | 16.46 | | | C |
| ANISOU | 523 | CD2 | LEU | A | 452 | 2212 | 2049 | 1991 | 728 | 12 | 131 | | C |
| ATOM | 524 | C | LEU | A | 452 | −8.671 | −31.065 | 7.312 | 1.00 | 18.87 | | | C |
| ANISOU | 524 | C | LEU | A | 452 | 2696 | 2085 | 2390 | 530 | 89 | −61 | | C |
| ATOM | 525 | O | LEU | A | 452 | −9.063 | −30.230 | 6.497 | 1.00 | 21.35 | | | O |
| ANISOU | 525 | O | LEU | A | 452 | 2986 | 2450 | 2674 | 451 | 86 | −101 | | O |
| ATOM | 526 | N | SER | A | 453 | −9.432 | −32.071 | 7.733 | 1.00 | 15.83 | | | N |
| ANISOU | 526 | N | SER | A | 453 | 2390 | 1566 | 2057 | 537 | 103 | −41 | | N |
| ATOM | 527 | CA | SER | A | 453 | −10.766 | −32.251 | 7.175 | 1.00 | 14.14 | | | C |
| ANISOU | 527 | CA | SER | A | 453 | 2232 | 1273 | 1867 | 444 | 114 | −83 | | C |
| ATOM | 528 | CB | SER | A | 453 | −10.728 | −33.109 | 5.913 | 1.00 | 13.36 | | | C |
| ANISOU | 528 | CB | SER | A | 453 | 2165 | 1114 | 1798 | 462 | 143 | −184 | | C |
| ATOM | 529 | OG | SER | A | 453 | −12.029 | −33.198 | 5.367 | 1.00 | 24.11 | | | O |
| ANISOU | 529 | OG | SER | A | 453 | 3566 | 2426 | 3170 | 358 | 147 | −228 | | O |
| ATOM | 530 | C | SER | A | 453 | −11.666 | −32.879 | 8.225 | 1.00 | 15.73 | | | C |
| ANISOU | 530 | C | SER | A | 453 | 2495 | 1371 | 2109 | 420 | 116 | −13 | | C |
| ATOM | 531 | O | SER | A | 453 | −11.399 | −33.978 | 8.715 | 1.00 | 13.54 | | | O |
| ANISOU | 531 | O | SER | A | 453 | 2267 | 993 | 1884 | 487 | 130 | 27 | | O |
| ATOM | 532 | N | HIS | A | 454 | −12.727 | −32.170 | 8.566 | 1.00 | 10.99 | | | N |
| ANISOU | 532 | N | HIS | A | 454 | 1891 | 801 | 1485 | 329 | 103 | 9 | | N |
| ATOM | 533 | CA | HIS | A | 454 | −13.663 | −32.652 | 9.564 | 1.00 | 13.72 | | | C |
| ANISOU | 533 | CA | HIS | A | 454 | 2280 | 1081 | 1853 | 292 | 108 | 81 | | C |
| ATOM | 534 | CB | HIS | A | 454 | −13.166 | −32.345 | 10.978 | 1.00 | 11.24 | | | C |
| ANISOU | 534 | CB | HIS | A | 454 | 1939 | 831 | 1502 | 344 | 90 | 176 | | C |
| ATOM | 535 | CG | HIS | A | 454 | −14.000 | −32.970 | 12.041 | 1.00 | 11.76 | | | C |
| ANISOU | 535 | CG | HIS | A | 454 | 2044 | 843 | 1581 | 318 | 101 | 266 | | C |
| ATOM | 536 | ND1 | HIS | A | 454 | −15.123 | −32.364 | 12.547 | 1.00 | 11.28 | | | N |
| ANISOU | 536 | ND1 | HIS | A | 454 | 1968 | 835 | 1484 | 238 | 99 | 286 | | N |
| ATOM | 537 | CE1 | HIS | A | 454 | −15.667 | −33.145 | 13.461 | 1.00 | 13.30 | | | C |
| ANISOU | 537 | CE1 | HIS | A | 454 | 2257 | 1043 | 1753 | 225 | 115 | 380 | | C |
| ATOM | 538 | NE2 | HIS | A | 454 | −14.934 | −34.240 | 13.566 | 1.00 | 13.02 | | | N |
| ANISOU | 538 | NE2 | HIS | A | 454 | 2267 | 909 | 1771 | 296 | 125 | 428 | | N |
| ATOM | 539 | CD2 | HIS | A | 454 | −13.891 | −34.159 | 12.676 | 1.00 | 12.86 | | | C |
| ANISOU | 539 | CD2 | HIS | A | 454 | 2236 | 884 | 1768 | 361 | 117 | 351 | | C |
| ATOM | 540 | C | HIS | A | 454 | −15.008 | −31.989 | 9.316 | 1.00 | 12.25 | | | C |
| ANISOU | 540 | C | HIS | A | 454 | 2083 | 925 | 1645 | 183 | 104 | 58 | | C |
| ATOM | 541 | O | HIS | A | 454 | −15.070 | −30.822 | 8.924 | 1.00 | 11.06 | | | O |
| ANISOU | 541 | O | HIS | A | 454 | 1883 | 870 | 1449 | 156 | 86 | 26 | | O |
| ATOM | 542 | N | GLU | A | 455 | −16.083 | −32.746 | 9.574 | 1.00 | 11.08 | | | N |
| ANISOU | 542 | N | GLU | A | 455 | 1980 | 696 | 1533 | 121 | 120 | 84 | | N |
| ATOM | 543 | CA | GLU | A | 455 | −17.435 | −32.256 | 9.311 | 1.00 | 13.47 | | | C |
| ANISOU | 543 | CA | GLU | A | 455 | 2264 | 1038 | 1817 | 20 | 118 | 64 | | C |
| ATOM | 544 | CB | GLU | A | 455 | −18.467 | −33.289 | 9.784 | 1.00 | 22.21 | | | C |
| ANISOU | 544 | CB | GLU | A | 455 | 3416 | 2055 | 2969 | −50 | 140 | 111 | | C |
| ATOM | 545 | CG | GLU | A | 455 | −18.118 | −34.731 | 9.418 | 1.00 | 40.45 | | | C |
| ANISOU | 545 | CG | GLU | A | 455 | 5802 | 4204 | 5363 | −38 | 160 | 94 | | C |
| ATOM | 546 | CD | GLU | A | 455 | −17.121 | −35.386 | 10.391 | 1.00 | 49.05 | | | C |
| ANISOU | 546 | CD | GLU | A | 455 | 6927 | 5227 | 6482 | 64 | 167 | 186 | | C |
| ATOM | 547 | OE1 | GLU | A | 455 | −17.548 | −35.788 | 11.503 | 1.00 | 62.42 | | | O |
| ANISOU | 547 | OE1 | GLU | A | 455 | 8638 | 6897 | 8183 | 46 | 177 | 297 | | O |
| ATOM | 548 | OE2 | GLU | A | 455 | −15.916 | −35.504 | 10.048 | 1.00 | 25.36 | | | O |
| ANISOU | 548 | OE2 | GLU | A | 455 | 3930 | 2214 | 3491 | 164 | 164 | 154 | | O |
| ATOM | 549 | C | GLU | A | 455 | −17.717 | −30.914 | 9.979 | 1.00 | 12.09 | | | C |
| ANISOU | 549 | C | GLU | A | 455 | 2030 | 986 | 1577 | 16 | 99 | 94 | | C |
| ATOM | 550 | O | GLU | A | 455 | −18.554 | −30.150 | 9.491 | 1.00 | 9.64 | | | O |
| ANISOU | 550 | O | GLU | A | 455 | 1686 | 735 | 1243 | −37 | 91 | 60 | | O |
| ATOM | 551 | N | LYS | A | 456 | −17.056 | −30.613 | 11.105 | 1.00 | 10.34 | | | N |
| ANISOU | 551 | N | LYS | A | 456 | 1795 | 807 | 1327 | 77 | 90 | 153 | | N |
| ATOM | 552 | CA | LYS | A | 456 | −17.349 | −29.405 | 11.872 | 1.00 | 9.20 | | | C |
| ANISOU | 552 | CA | LYS | A | 456 | 1603 | 769 | 1125 | 75 | 72 | 167 | | C |
| ATOM | 553 | CB | LYS | A | 456 | −17.610 | −29.761 | 13.343 | 1.00 | 9.72 | | | C |
| ANISOU | 553 | CB | LYS | A | 456 | 1672 | 861 | 1161 | 89 | 81 | 253 | | C |
| ATOM | 554 | CG | LYS | A | 456 | −18.759 | −30.780 | 13.517 | 1.00 | 10.40 | | | C |
| ANISOU | 554 | CG | LYS | A | 456 | 1788 | 886 | 1276 | 23 | 111 | 304 | | C |
| ATOM | 555 | CD | LYS | A | 456 | −20.085 | −30.273 | 12.897 | 1.00 | 11.57 | | | C |
| ANISOU | 555 | CD | LYS | A | 456 | 1908 | 1069 | 1419 | −58 | 116 | 256 | | C |
| ATOM | 556 | CE | LYS | A | 456 | −21.168 | −31.374 | 12.923 | 1.00 | 14.70 | | | C |
| ANISOU | 556 | CE | LYS | A | 456 | 2329 | 1406 | 1852 | −144 | 145 | 299 | | C |
| ATOM | 557 | NZ | LYS | A | 456 | −21.646 | −31.689 | 14.311 | 1.00 | 19.69 | | | N |
| ANISOU | 557 | NZ | LYS | A | 456 | 2950 | 2084 | 2448 | −154 | 165 | 403 | | N |
| ATOM | 558 | C | LYS | A | 456 | −16.240 | −28.363 | 11.755 | 1.00 | 8.70 | | | C |
| ANISOU | 558 | C | LYS | A | 456 | 1502 | 769 | 1033 | 122 | 45 | 133 | | C |
| ATOM | 559 | O | LYS | A | 456 | −16.198 | −27.405 | 12.531 | 1.00 | 10.63 | | | O |
| ANISOU | 559 | O | LYS | A | 456 | 1715 | 1088 | 1236 | 132 | 26 | 136 | | O |
| ATOM | 560 | N | LEU | A | 457 | −15.359 | −28.530 | 10.785 | 1.00 | 8.67 | | | N |
| ANISOU | 560 | N | LEU | A | 457 | 1500 | 743 | 1052 | 145 | 42 | 94 | | N |
| ATOM | 561 | CA | LEU | A | 457 | −14.255 | −27.623 | 10.514 | 1.00 | 10.10 | | | C |
| ANISOU | 561 | CA | LEU | A | 457 | 1639 | 988 | 1212 | 174 | 20 | 67 | | C |
| ATOM | 562 | CB | LEU | A | 457 | −12.940 | −28.383 | 10.594 | 1.00 | 10.61 | | | C |
| ANISOU | 562 | CB | LEU | A | 457 | 1702 | 1046 | 1285 | 249 | 22 | 83 | | C |
| ATOM | 563 | CG | LEU | A | 457 | −11.645 | −27.603 | 10.494 | 1.00 | 12.42 | | | C |
| ANISOU | 563 | CG | LEU | A | 457 | 1873 | 1360 | 1487 | 278 | −1 | 67 | | C |
| ATOM | 564 | CD1 | LEU | A | 457 | −11.497 | −26.805 | 11.770 | 1.00 | 12.70 | | | C |
| ANISOU | 564 | CD1 | LEU | A | 457 | 1878 | 1465 | 1480 | 277 | −30 | 93 | | C |
| ATOM | 565 | CD2 | LEU | A | 457 | −10.533 | −28.616 | 10.352 | 1.00 | 16.63 | | | C |
| ANISOU | 565 | CD2 | LEU | A | 457 | 2402 | 1883 | 2035 | 362 | 11 | 79 | | C |
| ATOM | 566 | C | LEU | A | 457 | −14.425 | −27.052 | 9.117 | 1.00 | 8.39 | | | C |
| ANISOU | 566 | C | LEU | A | 457 | 1406 | 780 | 1001 | 133 | 19 | 13 | | C |
| ATOM | 567 | O | LEU | A | 457 | −14.564 | −27.817 | 8.157 | 1.00 | 11.33 | | | O |
| ANISOU | 567 | O | LEU | A | 457 | 1799 | 1111 | 1394 | 124 | 37 | −18 | | O |
| ATOM | 568 | N | VAL | A | 458 | −14.408 | −25.722 | 8.987 | 1.00 | 7.56 | | | N |
| ANISOU | 568 | N | VAL | A | 458 | 1266 | 728 | 878 | 108 | −3 | 2 | | N |
| ATOM | 569 | CA | VAL | A | 458 | −14.543 | −25.135 | 7.656 | 1.00 | 7.36 | | | C |
| ANISOU | 569 | CA | VAL | A | 458 | 1221 | 722 | 852 | 72 | −5 | −26 | | C |
| ATOM | 570 | CB | VAL | A | 458 | −14.535 | −23.600 | 7.716 | 1.00 | 9.90 | | | C |
| ANISOU | 570 | CB | VAL | A | 458 | 1514 | 1079 | 1169 | 48 | −31 | −19 | | C |
| ATOM | 571 | CG1 | VAL | A | 458 | −14.529 | −23.016 | 6.281 | 1.00 | 7.98 | | | C |
| ANISOU | 571 | CG1 | VAL | A | 458 | 1247 | 863 | 922 | 14 | −33 | −23 | | C |
| ATOM | 572 | CG2 | VAL | A | 458 | −15.750 | −23.065 | 8.507 | 1.00 | 7.17 | | | C |
| ANISOU | 572 | CG2 | VAL | A | 458 | 1179 | 719 | 824 | 36 | −37 | −14 | | C |
| ATOM | 573 | C | VAL | A | 458 | −13.423 | −25.660 | 6.761 | 1.00 | 14.79 | | | C |
| ANISOU | 573 | C | VAL | A | 458 | 2147 | 1681 | 1790 | 99 | 6 | −48 | | C |
| ATOM | 574 | O | VAL | A | 458 | −12.233 | −25.563 | 7.097 | 1.00 | 13.59 | | | O |
| ANISOU | 574 | O | VAL | A | 458 | 1968 | 1564 | 1630 | 136 | −1 | −39 | | O |
| ATOM | 575 | N | GLN | A | 459 | −13.802 | −26.215 | 5.610 | 1.00 | 12.44 | | | N |
| ANISOU | 575 | N | GLN | A | 459 | 1859 | 1377 | 1491 | 80 | 24 | −84 | | N |
| ATOM | 576 | CA | GLN | A | 459 | −12.854 | −26.902 | 4.737 | 1.00 | 12.53 | | | C |
| ANISOU | 576 | CA | GLN | A | 459 | 1860 | 1410 | 1493 | 116 | 43 | −123 | | C |
| ATOM | 577 | CB | GLN | A | 459 | −13.585 | −27.872 | 3.807 | 1.00 | 14.21 | | | C |
| ANISOU | 577 | CB | GLN | A | 459 | 2104 | 1585 | 1709 | 96 | 64 | −182 | | C |
| ATOM | 578 | CG | GLN | A | 459 | −12.656 | −28.668 | 2.889 | 1.00 | 15.33 | | | C |
| ANISOU | 578 | CG | GLN | A | 459 | 2238 | 1747 | 1840 | 145 | 89 | −246 | | C |
| ATOM | 579 | CD | GLN | A | 459 | −11.782 | −29.627 | 3.663 | 1.00 | 24.94 | | | C |
| ANISOU | 579 | CD | GLN | A | 459 | 3481 | 2898 | 3096 | 231 | 103 | −241 | | C |
| ATOM | 580 | OE1 | GLN | A | 459 | −12.279 | −30.445 | 4.461 | 1.00 | 22.00 | | | O |
| ANISOU | 580 | OE1 | GLN | A | 459 | 3167 | 2419 | 2772 | 241 | 107 | −224 | | O |
| ATOM | 581 | NE2 | GLN | A | 459 | −10.464 | −29.529 | 3.451 | 1.00 | 24.27 | | | N |
| ANISOU | 581 | NE2 | GLN | A | 459 | 3348 | 2886 | 2990 | 296 | 110 | −246 | | N |
| ATOM | 582 | C | GLN | A | 459 | −12.087 | −25.900 | 3.896 | 1.00 | 9.80 | | | C |
| ANISOU | 582 | C | GLN | A | 459 | 1453 | 1159 | 1111 | 100 | 35 | −117 | | C |
| ATOM | 583 | O | GLN | A | 459 | −12.689 | −25.067 | 3.221 | 1.00 | 12.36 | | | O |
| ANISOU | 583 | O | GLN | A | 459 | 1761 | 1520 | 1417 | 47 | 25 | −105 | | O |
| ATOM | 584 | N | LEU | A | 460 | −10.759 | −25.963 | 3.956 | 1.00 | 8.63 | | | N |
| ANISOU | 584 | N | LEU | A | 460 | 1267 | 1060 | 952 | 146 | 40 | −115 | | N |
| ATOM | 585 | CA | LEU | A | 460 | −9.927 | −25.173 | 3.063 | 1.00 | 13.84 | | | C |
| ANISOU | 585 | CA | LEU | A | 460 | 1860 | 1824 | 1574 | 124 | 40 | −105 | | C |
| ATOM | 586 | CB | LEU | A | 460 | −8.511 | −25.017 | 3.626 | 1.00 | 14.05 | | | C |
| ANISOU | 586 | CB | LEU | A | 460 | 1833 | 1910 | 1596 | 163 | 34 | −84 | | C |
| ATOM | 587 | CG | LEU | A | 460 | −7.489 | −24.355 | 2.695 | 1.00 | 11.22 | | | C |
| ANISOU | 587 | CG | LEU | A | 460 | 1392 | 1676 | 1195 | 138 | 42 | −70 | | C |
| ATOM | 588 | CD1 | LEU | A | 460 | −7.887 | −22.915 | 2.416 | 1.00 | 13.69 | | | C |
| ANISOU | 588 | CD1 | LEU | A | 460 | 1691 | 2001 | 1509 | 43 | 18 | −16 | | C |
| ATOM | 589 | CD2 | LEU | A | 460 | −6.094 | −24.429 | 3.299 | 1.00 | 12.52 | | | C |
| ANISOU | 589 | CD2 | LEU | A | 460 | 1493 | 1913 | 1352 | 183 | 38 | −58 | | C |
| ATOM | 590 | C | LEU | A | 460 | −9.900 | −25.882 | 1.720 | 1.00 | 17.30 | | | C |
| ANISOU | 590 | C | LEU | A | 460 | 2289 | 2309 | 1977 | 138 | 71 | −162 | | C |
| ATOM | 591 | O | LEU | A | 460 | −9.612 | −27.081 | 1.658 | 1.00 | 15.32 | | | O |
| ANISOU | 591 | O | LEU | A | 460 | 2061 | 2026 | 1735 | 202 | 95 | −218 | | O |
| ATOM | 592 | N | TYR | A | 461 | −10.259 | −25.163 | 0.658 | 1.00 | 9.38 | | | N |
| ANISOU | 592 | N | TYR | A | 461 | 1256 | 1376 | 931 | 80 | 69 | −150 | | N |
| ATOM | 593 | CA | TYR | A | 461 | −10.163 | −25.713 | −0.686 | 1.00 | 10.03 | | | C |
| ANISOU | 593 | CA | TYR | A | 461 | 1315 | 1540 | 956 | 88 | 97 | −208 | | C |
| ATOM | 594 | CB | TYR | A | 461 | −11.313 | −25.192 | −1.541 | 1.00 | 13.61 | | | C |
| ANISOU | 594 | CB | TYR | A | 461 | 1770 | 2029 | 1374 | 20 | 85 | −195 | | C |
| ATOM | 595 | CG | TYR | A | 461 | −12.695 | −25.708 | −1.196 | 1.00 | 16.93 | | | C |
| ANISOU | 595 | CG | TYR | A | 461 | 2253 | 2353 | 1828 | −1 | 74 | −226 | | C |
| ATOM | 596 | CD1 | TYR | A | 461 | −12.881 | −26.969 | −0.637 | 1.00 | 18.96 | | | C |
| ANISOU | 596 | CD1 | TYR | A | 461 | 2568 | 2508 | 2130 | 35 | 88 | −293 | | C |
| ATOM | 597 | CE1 | TYR | A | 461 | −14.158 | −27.438 | −0.337 | 1.00 | 19.08 | | | C |
| ANISOU | 597 | CE1 | TYR | A | 461 | 2631 | 2443 | 2174 | −2 | 79 | −313 | | C |
| ATOM | 598 | CZ | TYR | A | 461 | −15.261 | −26.649 | −0.624 | 1.00 | 17.16 | | | C |
| ANISOU | 598 | CZ | TYR | A | 461 | 2370 | 2240 | 1909 | −64 | 56 | −275 | | C |
| ATOM | 599 | OH | TYR | A | 461 | −16.528 | −27.104 | −0.354 | 1.00 | 17.54 | | | O |
| ANISOU | 599 | OH | TYR | A | 461 | 2450 | 2236 | 1980 | −105 | 49 | −294 | | O |
| ATOM | 600 | CE2 | TYR | A | 461 | −15.103 | −25.399 | −1.183 | 1.00 | 16.11 | | | C |
| ANISOU | 600 | CE2 | TYR | A | 461 | 2183 | 2202 | 1734 | −83 | 41 | −208 | | C |
| ATOM | 601 | CD2 | TYR | A | 461 | −13.820 | −24.935 | −1.464 | 1.00 | 19.07 | | | C |
| ANISOU | 601 | CD2 | TYR | A | 461 | 2519 | 2639 | 2086 | −58 | 50 | −181 | | C |
| ATOM | 602 | C | TYR | A | 461 | −8.837 | −25.367 | −1.354 | 1.00 | 12.78 | | | C |
| ANISOU | 602 | C | TYR | A | 461 | 1580 | 2025 | 1251 | 104 | 115 | −195 | | C |
| ATOM | 603 | O | TYR | A | 461 | −8.356 | −26.136 | −2.199 | 1.00 | 13.97 | | | O |
| ANISOU | 603 | O | TYR | A | 461 | 1706 | 2249 | 1354 | 149 | 148 | −266 | | O |
| ATOM | 604 | N | GLY | A | 462 | −8.243 | −24.236 | −1.005 | 1.00 | 12.32 | | | N |
| ANISOU | 604 | N | GLY | A | 462 | 1476 | 2006 | 1198 | 64 | 97 | −112 | | N |
| ATOM | 605 | CA | GLY | A | 462 | −6.960 | −23.856 | −1.563 | 1.00 | 15.90 | | | C |
| ANISOU | 605 | CA | GLY | A | 462 | 1839 | 2599 | 1602 | 63 | 114 | −85 | | C |
| ATOM | 606 | C | GLY | A | 462 | −6.752 | −22.360 | −1.481 | 1.00 | 15.42 | | | C |
| ANISOU | 606 | C | GLY | A | 462 | 1740 | 2567 | 1552 | −28 | 87 | 19 | | C |
| ATOM | 607 | O | GLY | A | 462 | −7.560 | −21.622 | −0.916 | 1.00 | 14.68 | | | O |
| ANISOU | 607 | O | GLY | A | 462 | 1695 | 2373 | 1509 | −74 | 55 | 61 | | O |
| ATOM | 608 | N | AVAL | A | 463 | −5.627 | −21.929 | −2.048 | 0.64 | 13.48 | | | N |
| ANISOU | 608 | N | AVAL | A | 463 | 1404 | 2459 | 1260 | −52 | 104 | 58 | | N |
| ATOM | 609 | CA | AVAL | A | 463 | −5.238 | −20.527 | −2.056 | 0.64 | 12.23 | | | C |
| ANISOU | 609 | CA | AVAL | A | 463 | 1201 | 2328 | 1116 | −151 | 82 | 162 | | C |
| ATOM | 610 | CB | AVAL | A | 463 | −4.014 | −20.256 | −1.145 | 0.64 | 12.62 | | | C |
| ANISOU | 610 | CB | AVAL | A | 463 | 1196 | 2405 | 1195 | −161 | 69 | 175 | | C |
| ATOM | 611 | CG1 | AVAL | A | 463 | −4.368 | −20.494 | 0.327 | 0.64 | 14.84 | | | C |
| ANISOU | 611 | CG1 | AVAL | A | 463 | 1546 | 2545 | 1547 | −124 | 36 | 134 | | C |
| ATOM | 612 | CG2 | AVAL | A | 463 | −2.847 | −21.133 | −1.557 | 0.64 | 14.24 | | | C |
| ANISOU | 612 | CG2 | AVAL | A | 463 | 1312 | 2762 | 1334 | −90 | 109 | 131 | | C |
| ATOM | 613 | C | AVAL | A | 463 | −4.937 | −20.113 | −3.491 | 0.64 | 14.34 | | | C |
| ANISOU | 613 | C | AVAL | A | 463 | 1395 | 2752 | 1300 | −199 | 110 | 220 | | C |
| ATOM | 614 | O | AVAL | A | 463 | −4.591 | −20.939 | −4.340 | 0.64 | 16.97 | | | O |
| ANISOU | 614 | O | AVAL | A | 463 | 1683 | 3211 | 1553 | −145 | 150 | 164 | | O |
| ATOM | 615 | N | BVAL | A | 463 | −5.645 | −21.924 | −2.082 | 0.36 | 13.44 | | | N |
| ANISOU | 615 | N | BVAL | A | 463 | 1398 | 2455 | 1253 | −53 | 104 | 58 | | N |
| ATOM | 616 | CA | BVAL | A | 463 | −5.227 | −20.530 | −2.049 | 0.36 | 12.67 | | | C |
| ANISOU | 616 | CA | BVAL | A | 463 | 1257 | 2385 | 1173 | −151 | 82 | 162 | | C |
| ATOM | 617 | CB | BVAL | A | 463 | −4.063 | −20.305 | −1.058 | 0.36 | 13.39 | | | C |
| ANISOU | 617 | CB | BVAL | A | 463 | 1300 | 2491 | 1297 | −156 | 68 | 170 | | C |
| ATOM | 618 | CG1 | BVAL | A | 463 | −3.598 | −18.867 | −1.113 | 0.36 | 12.71 | | | C |
| ANISOU | 618 | CG1 | BVAL | A | 463 | 1168 | 2427 | 1235 | −277 | 46 | 269 | | C |
| ATOM | 619 | CG2 | BVAL | A | 463 | −4.482 | −20.685 | 0.359 | 0.36 | 14.00 | | | C |
| ANISOU | 619 | CG2 | BVAL | A | 463 | 1451 | 2429 | 1441 | −109 | 38 | 123 | | C |
| ATOM | 620 | C | BVAL | A | 463 | −4.819 | −20.092 | −3.450 | 0.36 | 14.69 | | | C |
| ANISOU | 620 | C | BVAL | A | 463 | 1433 | 2803 | 1346 | −200 | 111 | 222 | | C |
| ATOM | 621 | O | BVAL | A | 463 | −4.274 | −20.877 | −4.233 | 0.36 | 15.32 | | | O |
| ANISOU | 621 | O | BVAL | A | 463 | 1456 | 3019 | 1345 | −147 | 151 | 171 | | O |
| ATOM | 622 | N | CYS | A | 464 | −5.066 | −18.820 | −3.758 | 1.00 | 13.13 | | | N |
| ANISOU | 622 | N | CYS | A | 464 | 1230 | 2593 | 1168 | −298 | 90 | 333 | | N |
| ATOM | 623 | CA | CYS | A | 464 | −4.673 | −18.232 | −5.041 | 1.00 | 14.16 | | | C |
| ANISOU | 623 | CA | CYS | A | 464 | 1281 | 2880 | 1220 | −361 | 114 | 425 | | C |
| ATOM | 624 | CB | CYS | A | 464 | −5.827 | −17.484 | −5.706 | 1.00 | 14.16 | | | C |
| ANISOU | 624 | CB | CYS | A | 464 | 1324 | 2838 | 1219 | −407 | 96 | 511 | | C |
| ATOM | 625 | SG | CYS | A | 464 | −7.334 | −18.451 | −5.952 | 1.00 | 19.66 | | | S |
| ANISOU | 625 | SG | CYS | A | 464 | 2099 | 3484 | 1889 | −326 | 93 | 414 | | S |
| ATOM | 626 | C | CYS | A | 464 | −3.513 | −17.288 | −4.755 | 1.00 | 17.21 | | | C |
| ANISOU | 626 | C | CYS | A | 464 | 1595 | 3309 | 1636 | −451 | 108 | 512 | | C |
| ATOM | 627 | O | CYS | A | 464 | −3.712 | −16.221 | −4.165 | 1.00 | 18.52 | | | O |
| ANISOU | 627 | O | CYS | A | 464 | 1800 | 3347 | 1891 | −529 | 70 | 581 | | O |
| ATOM | 628 | N | THR | A | 465 | −2.309 | −17.676 | −5.176 | 1.00 | 19.76 | | | N |
| ANISOU | 628 | N | THR | A | 465 | 1811 | 3813 | 1886 | −442 | 145 | 501 | | N |
| ATOM | 629 | CA | THR | A | 465 | −1.087 | −17.017 | −4.738 | 1.00 | 26.72 | | | C |
| ANISOU | 629 | CA | THR | A | 465 | 2608 | 4752 | 2792 | −521 | 138 | 558 | | C |
| ATOM | 630 | CB | THR | A | 465 | −0.164 | −18.021 | −4.030 | 1.00 | 33.26 | | | C |
| ANISOU | 630 | CB | THR | A | 465 | 3386 | 5646 | 3607 | −427 | 151 | 452 | | C |
| ATOM | 631 | OG1 | THR | A | 465 | 0.152 | −19.080 | −4.941 | 1.00 | 41.46 | | | O |
| ANISOU | 631 | OG1 | THR | A | 465 | 4366 | 6849 | 4538 | −328 | 205 | 387 | | O |
| ATOM | 632 | CG2 | THR | A | 465 | −0.861 | −18.609 | −2.823 | 1.00 | 36.55 | | | C |
| ANISOU | 632 | CG2 | THR | A | 465 | 3912 | 5876 | 4100 | −346 | 117 | 359 | | C |
| ATOM | 633 | C | THR | A | 465 | −0.295 | −16.349 | −5.851 | 1.00 | 24.81 | | | C |
| ANISOU | 633 | C | THR | A | 465 | 2262 | 4685 | 2481 | −607 | 163 | 668 | | C |
| ATOM | 634 | O | THR | A | 465 | 0.725 | −15.711 | −5.558 | 1.00 | 23.43 | | | O |
| ANISOU | 634 | O | THR | A | 465 | 2031 | 4537 | 2334 | −680 | 144 | 712 | | O |
| ATOM | 635 | N | LYS | A | 466 | −0.709 | −16.469 | −7.110 | 1.00 | 21.59 | | | N |
| ANISOU | 635 | N | LYS | A | 466 | 1854 | 4354 | 1993 | −580 | 177 | 688 | | N |
| ATOM | 636 | CA | LYS | A | 466 | 0.142 | −15.878 | −8.137 | 1.00 | 35.47 | | | C |
| ANISOU | 636 | CA | LYS | A | 466 | 3533 | 6247 | 3695 | −633 | 175 | 770 | | C |
| ATOM | 637 | CB | LYS | A | 466 | −0.204 | −16.483 | −9.504 | 1.00 | 33.22 | | | C |
| ANISOU | 637 | CB | LYS | A | 466 | 3224 | 6100 | 3297 | −566 | 197 | 740 | | C |
| ATOM | 638 | CG | LYS | A | 466 | 0.002 | −17.990 | −9.607 | 0.00 | 26.19 | | | C |
| ANISOU | 638 | CG | LYS | A | 466 | 2309 | 5309 | 2334 | −434 | 237 | 581 | | C |
| ATOM | 639 | CD | LYS | A | 466 | 1.474 | −18.349 | −9.756 | 0.00 | 23.22 | | | C |
| ANISOU | 639 | CD | LYS | A | 466 | 1826 | 5095 | 1903 | −404 | 256 | 550 | | C |
| ATOM | 640 | CE | LYS | A | 466 | 1.654 | −19.829 | −10.066 | 0.00 | 21.78 | | | C |
| ANISOU | 640 | CE | LYS | A | 466 | 1625 | 4999 | 1653 | −257 | 293 | 390 | | C |
| ATOM | 641 | NZ | LYS | A | 466 | 1.103 | −20.697 | −8.988 | 0.00 | 20.20 | | | N |
| ANISOU | 641 | NZ | LYS | A | 466 | 1501 | 4658 | 1514 | −171 | 304 | 281 | | N |
| ATOM | 642 | C | LYS | A | 466 | 0.030 | −14.358 | −8.185 | 1.00 | 37.14 | | | C |
| ANISOU | 642 | C | LYS | A | 466 | 3775 | 6348 | 3987 | −761 | 135 | 915 | | C |
| ATOM | 643 | O | LYS | A | 466 | 0.705 | −13.726 | −9.001 | 1.00 | 38.52 | | | O |
| ANISOU | 643 | O | LYS | A | 466 | 3889 | 6621 | 4126 | −819 | 132 | 1002 | | O |
| ATOM | 644 | N | GLN | A | 467 | −0.756 | −13.760 | −7.294 | 1.00 | 42.45 | | | N |
| ANISOU | 644 | N | GLN | A | 467 | 4539 | 6815 | 4773 | −802 | 104 | 938 | | N |
| ATOM | 645 | CA | GLN | A | 467 | −1.354 | −12.453 | −7.506 | 1.00 | 45.41 | | | C |
| ANISOU | 645 | CA | GLN | A | 467 | 4977 | 7046 | 5230 | −880 | 68 | 1058 | | C |
| ATOM | 646 | CB | GLN | A | 467 | −2.771 | −12.607 | −8.049 | 1.00 | 54.95 | | | C |
| ANISOU | 646 | CB | GLN | A | 467 | 6253 | 8204 | 6422 | −820 | 64 | 1069 | | C |
| ATOM | 647 | CG | GLN | A | 467 | −3.604 | −13.522 | −7.141 | 1.00 | 57.92 | | | C |
| ANISOU | 647 | CG | GLN | A | 467 | 6693 | 8498 | 6815 | −747 | 71 | 959 | | C |
| ATOM | 648 | CD | GLN | A | 467 | −5.062 | −13.586 | −7.529 | 1.00 | 61.54 | | | C |
| ANISOU | 648 | CD | GLN | A | 467 | 7224 | 8892 | 7267 | −696 | 59 | 970 | | C |
| ATOM | 649 | OE1 | GLN | A | 467 | −5.414 | −14.102 | −8.589 | 1.00 | 70.09 | | | O |
| ANISOU | 649 | OE1 | GLN | A | 467 | 8277 | 10105 | 8250 | −647 | 75 | 956 | | O |
| ATOM | 650 | NE2 | GLN | A | 467 | −5.922 | −13.058 | −6.670 | 1.00 | 57.51 | | | N |
| ANISOU | 650 | NE2 | GLN | A | 467 | 6805 | 8184 | 6862 | −706 | 27 | 986 | | N |
| ATOM | 651 | C | GLN | A | 467 | −1.422 | −11.678 | −6.199 | 1.00 | 40.64 | | | C |
| ANISOU | 651 | C | GLN | A | 467 | 4441 | 6233 | 4767 | −948 | 31 | 1059 | | C |
| ATOM | 652 | O | GLN | A | 467 | −1.280 | −12.231 | −5.108 | 1.00 | 43.96 | | | O |
| ANISOU | 652 | O | GLN | A | 467 | 4868 | 6616 | 5218 | −928 | 29 | 966 | | O |
| ATOM | 653 | N | ARG | A | 468 | −1.687 | −10.387 | −6.318 | 1.00 | 32.97 | | | N |
| ANISOU | 653 | N | ARG | A | 468 | 3521 | 5122 | 3885 | −1021 | 1 | 1157 | | N |
| ATOM | 654 | CA | ARG | A | 468 | −1.982 | −9.571 | −5.159 | 1.00 | 34.28 | | | C |
| ANISOU | 654 | CA | ARG | A | 468 | 3771 | 5060 | 4192 | −1071 | −38 | 1142 | | C |
| ATOM | 655 | CB | ARG | A | 468 | −0.909 | −8.503 | −4.952 | 1.00 | 40.57 | | | C |
| ANISOU | 655 | CB | ARG | A | 468 | 4541 | 5819 | 5056 | −1190 | −54 | 1190 | | C |
| ATOM | 656 | CG | ARG | A | 468 | 0.490 | −9.056 | −4.732 | 0.00 | 38.24 | | | C |
| ANISOU | 656 | CG | ARG | A | 468 | 4136 | 5701 | 4694 | −1222 | −38 | 1141 | | C |
| ATOM | 657 | CD | ARG | A | 468 | 1.504 | −7.930 | −4.606 | 0.00 | 37.48 | | | C |
| ANISOU | 657 | CD | ARG | A | 468 | 4010 | 5570 | 4661 | −1351 | −54 | 1196 | | C |
| ATOM | 658 | NE | ARG | A | 468 | 2.852 | −8.428 | −4.344 | 0.00 | 36.58 | | | N |
| ANISOU | 658 | NE | ARG | A | 468 | 3783 | 5633 | 4481 | −1378 | −43 | 1149 | | N |
| ATOM | 659 | CZ | ARG | A | 468 | 3.925 | −7.652 | −4.228 | 0.00 | 37.43 | | | C |
| ANISOU | 659 | CZ | ARG | A | 468 | 3840 | 5762 | 4620 | −1493 | −53 | 1184 | | C |
| ATOM | 660 | NH1 | ARG | A | 468 | 3.811 | −6.337 | −4.354 | 0.00 | 38.50 | | | N |
| ANISOU | 660 | NH1 | ARG | A | 468 | 4031 | 5739 | 4859 | −1595 | −71 | 1265 | | N |
| ATOM | 661 | NH2 | ARG | A | 468 | 5.112 | −8.191 | −3.989 | 0.00 | 37.65 | | | N |
| ANISOU | 661 | NH2 | ARG | A | 468 | 3759 | 5971 | 4578 | −1502 | −43 | 1138 | | N |
| ATOM | 662 | C | ARG | A | 468 | −3.341 | −8.938 | −5.383 | 1.00 | 32.89 | | | C |
| ANISOU | 662 | C | ARG | A | 468 | 3695 | 4721 | 4081 | −1038 | −58 | 1196 | | C |
| ATOM | 663 | O | ARG | A | 468 | −3.568 | −8.312 | −6.415 | 1.00 | 37.38 | | | O |
| ANISOU | 663 | O | ARG | A | 468 | 4257 | 5310 | 4634 | −1047 | −56 | 1303 | | O |
| ATOM | 664 | N | PRO | A | 469 | −4.260 | −9.102 | −4.424 | 1.00 | 21.29 | | | N |
| ANISOU | 664 | N | PRO | A | 469 | 2310 | 3098 | 2680 | −991 | −79 | 1125 | | N |
| ATOM | 665 | CA | PRO | A | 469 | −4.113 | −9.840 | −3.165 | 1.00 | 21.56 | | | C |
| ANISOU | 665 | CA | PRO | A | 469 | 2354 | 3104 | 2733 | −978 | −87 | 1004 | | C |
| ATOM | 666 | CB | PRO | A | 469 | −5.209 | −9.236 | −2.286 | 1.00 | 21.21 | | | C |
| ANISOU | 666 | CB | PRO | A | 469 | 2426 | 2825 | 2806 | −954 | −125 | 973 | | C |
| ATOM | 667 | CG | PRO | A | 469 | −6.244 | −8.793 | −3.256 | 1.00 | 20.11 | | | C |
| ANISOU | 667 | CG | PRO | A | 469 | 2327 | 2650 | 2662 | −902 | −122 | 1063 | | C |
| ATOM | 668 | CD | PRO | A | 469 | −5.537 | −8.367 | −4.491 | 1.00 | 19.64 | | | C |
| ANISOU | 668 | CD | PRO | A | 469 | 2197 | 2718 | 2547 | −951 | −102 | 1170 | | C |
| ATOM | 669 | C | PRO | A | 469 | −4.322 | −11.342 | −3.312 | 1.00 | 22.54 | | | C |
| ANISOU | 669 | C | PRO | A | 469 | 2439 | 3378 | 2745 | −890 | −52 | 934 | | C |
| ATOM | 670 | O | PRO | A | 469 | −5.033 | −11.774 | −4.210 | 1.00 | 25.58 | | | O |
| ANISOU | 670 | O | PRO | A | 469 | 2827 | 3834 | 3057 | −829 | −31 | 962 | | O |
| ATOM | 671 | N | ILE | A | 470 | −3.721 | −12.137 | −2.430 | 1.00 | 17.44 | | | N |
| ANISOU | 671 | N | ILE | A | 470 | 1771 | 2771 | 2085 | −847 | −53 | 809 | | N |
| ATOM | 672 | CA | ILE | A | 470 | −3.957 | −13.571 | −2.477 | 1.00 | 19.52 | | | C |
| ANISOU | 672 | CA | ILE | A | 470 | 2028 | 3126 | 2262 | −719 | −26 | 702 | | C |
| ATOM | 673 | CB | ILE | A | 470 | −2.886 | −14.337 | −1.682 | 1.00 | 19.46 | | | C |
| ANISOU | 673 | CB | ILE | A | 470 | 1962 | 3201 | 2233 | −687 | −22 | 606 | | C |
| ATOM | 674 | CG1 | ILE | A | 470 | −2.952 | −13.962 | −0.214 | 1.00 | 21.72 | | | C |
| ANISOU | 674 | CG1 | ILE | A | 470 | 2306 | 3336 | 2611 | −701 | −68 | 542 | | C |
| ATOM | 675 | CD1 | ILE | A | 470 | −1.805 | −14.519 | 0.586 | 1.00 | 30.56 | | | C |
| ANISOU | 675 | CD1 | ILE | A | 470 | 3353 | 4552 | 3706 | −684 | −73 | 472 | | C |
| ATOM | 676 | CG2 | ILE | A | 470 | −1.485 | −14.003 | −2.219 | 1.00 | 17.71 | | | C |
| ANISOU | 676 | CG2 | ILE | A | 470 | 1613 | 3150 | 1966 | −777 | −0 | 675 | | C |
| ATOM | 677 | C | ILE | A | 470 | −5.355 | −13.866 | −1.949 | 1.00 | 16.76 | | | C |
| ANISOU | 677 | C | ILE | A | 470 | 1788 | 2627 | 1954 | −634 | −46 | 637 | | C |
| ATOM | 678 | O | ILE | A | 470 | −5.921 | −13.091 | −1.166 | 1.00 | 17.51 | | | O |
| ANISOU | 678 | O | ILE | A | 470 | 1956 | 2554 | 2144 | −655 | −83 | 636 | | O |
| ATOM | 679 | N | PHE | A | 471 | −5.915 | −15.000 | −2.381 | 1.00 | 14.18 | | | N |
| ANISOU | 679 | N | PHE | A | 471 | 1469 | 2365 | 1552 | −538 | −21 | 575 | | N |
| ATOM | 680 | CA | PHE | A | 471 | −7.242 | −15.462 | −1.987 | 1.00 | 12.70 | | | C |
| ANISOU | 680 | CA | PHE | A | 471 | 1368 | 2070 | 1387 | −462 | −34 | 513 | | C |
| ATOM | 681 | CB | PHE | A | 471 | −8.142 | −15.785 | −3.204 | 1.00 | 13.48 | | | C |
| ANISOU | 681 | CB | PHE | A | 471 | 1467 | 2241 | 1413 | −432 | −16 | 543 | | C |
| ATOM | 682 | CG | PHE | A | 471 | −8.380 | −14.643 | −4.147 | 1.00 | 13.83 | | | C |
| ANISOU | 682 | CG | PHE | A | 471 | 1494 | 2306 | 1456 | −500 | −23 | 687 | | C |
| ATOM | 683 | CD1 | PHE | A | 471 | −8.141 | −13.331 | −3.779 | 1.00 | 15.80 | | | C |
| ANISOU | 683 | CD1 | PHE | A | 471 | 1759 | 2444 | 1798 | −576 | −49 | 778 | | C |
| ATOM | 684 | CE1 | PHE | A | 471 | −8.377 | −12.292 | −4.673 | 1.00 | 20.22 | | | C |
| ANISOU | 684 | CE1 | PHE | A | 471 | 2310 | 3008 | 2366 | −634 | −55 | 930 | | C |
| ATOM | 685 | CZ | PHE | A | 471 | −8.860 | −12.566 | −5.949 | 1.00 | 24.83 | | | C |
| ANISOU | 685 | CZ | PHE | A | 471 | 2855 | 3735 | 2845 | −613 | −36 | 993 | | C |
| ATOM | 686 | CE2 | PHE | A | 471 | −9.105 | −13.871 | −6.323 | 1.00 | 16.91 | | | C |
| ANISOU | 686 | CE2 | PHE | A | 471 | 1830 | 2858 | 1738 | −541 | −11 | 884 | | C |
| ATOM | 687 | CD2 | PHE | A | 471 | −8.877 | −14.898 | −5.423 | 1.00 | 15.64 | | | C |
| ANISOU | 687 | CD2 | PHE | A | 471 | 1691 | 2665 | 1589 | −486 | −4 | 731 | | C |
| ATOM | 688 | C | PHE | A | 471 | −7.115 | −16.734 | −1.160 | 1.00 | 17.05 | | | C |
| ANISOU | 688 | C | PHE | A | 471 | 1935 | 2621 | 1924 | −376 | −25 | 388 | | C |
| ATOM | 689 | O | PHE | A | 471 | −6.250 | −17.573 | −1.427 | 1.00 | 14.94 | | | O |
| ANISOU | 689 | O | PHE | A | 471 | 1609 | 2470 | 1595 | −343 | 3 | 347 | | O |
| ATOM | 690 | N | ILE | A | 472 | −7.964 | −16.866 | −0.146 | 1.00 | 16.10 | | | N |
| ANISOU | 690 | N | ILE | A | 472 | 1890 | 2370 | 1858 | −336 | −48 | 334 | | N |
| ATOM | 691 | CA | ILE | A | 472 | −8.181 | −18.123 | 0.564 | 1.00 | 11.13 | | | C |
| ANISOU | 691 | CA | ILE | A | 472 | 1290 | 1721 | 1216 | −252 | −39 | 237 | | C |
| ATOM | 692 | CB | ILE | A | 472 | −8.087 | −17.953 | 2.092 | 1.00 | 13.50 | | | C |
| ANISOU | 692 | CB | ILE | A | 472 | 1625 | 1928 | 1575 | −242 | −68 | 196 | | C |
| ATOM | 693 | CG1 | ILE | A | 472 | −6.786 | −17.266 | 2.499 | 1.00 | 11.37 | | | C |
| ANISOU | 693 | CG1 | ILE | A | 472 | 1298 | 1700 | 1322 | −302 | −84 | 217 | | C |
| ATOM | 694 | CD1 | ILE | A | 472 | −6.682 | −17.053 | 4.047 | 1.00 | 13.20 | | | C |
| ANISOU | 694 | CD1 | ILE | A | 472 | 1558 | 1861 | 1597 | −297 | −119 | 165 | | C |
| ATOM | 695 | CG2 | ILE | A | 472 | −8.225 | −19.307 | 2.776 | 1.00 | 11.73 | | | C |
| ANISOU | 695 | CG2 | ILE | A | 472 | 1427 | 1696 | 1334 | −156 | −55 | 121 | | C |
| ATOM | 696 | C | ILE | A | 472 | −9.573 | −18.591 | 0.173 | 1.00 | 11.68 | | | C |
| ANISOU | 696 | C | ILE | A | 472 | 1413 | 1751 | 1274 | −216 | −34 | 216 | | C |
| ATOM | 697 | O | ILE | A | 472 | −10.539 | −17.842 | 0.347 | 1.00 | 13.32 | | | O |
| ANISOU | 697 | O | ILE | A | 472 | 1661 | 1877 | 1523 | −233 | −56 | 250 | | O |
| ATOM | 698 | N | ILE | A | 473 | −9.680 | −19.812 | −0.362 | 1.00 | 11.07 | | | N |
| ANISOU | 698 | N | ILE | A | 473 | 1331 | 1733 | 1142 | −165 | −5 | 157 | | N |
| ATOM | 699 | CA | ILE | A | 473 | −10.934 | −20.371 | −0.867 | 1.00 | 9.34 | | | C |
| ANISOU | 699 | CA | ILE | A | 473 | 1149 | 1500 | 900 | −146 | −0 | 127 | | C |
| ATOM | 700 | CB | ILE | A | 473 | −10.768 | −20.943 | −2.291 | 1.00 | 10.68 | | | C |
| ANISOU | 700 | CB | ILE | A | 473 | 1274 | 1803 | 982 | −143 | 29 | 107 | | C |
| ATOM | 701 | CG1 | ILE | A | 473 | −9.987 | −19.981 | −3.186 | 1.00 | 12.86 | | | C |
| ANISOU | 701 | CG1 | ILE | A | 473 | 1479 | 2191 | 1215 | −195 | 34 | 197 | | C |
| ATOM | 702 | CD1 | ILE | A | 473 | −10.675 | −18.645 | −3.375 | 1.00 | 13.74 | | | C |
| ANISOU | 702 | CD1 | ILE | A | 473 | 1600 | 2263 | 1357 | −250 | 5 | 307 | | C |
| ATOM | 703 | CG2 | ILE | A | 473 | −12.124 | −21.232 | −2.920 | 1.00 | 9.90 | | | C |
| ANISOU | 703 | CG2 | ILE | A | 473 | 1200 | 1709 | 852 | −148 | 23 | 88 | | C |
| ATOM | 704 | C | ILE | A | 473 | −11.395 | −21.463 | 0.096 | 1.00 | 8.82 | | | C |
| ANISOU | 704 | C | ILE | A | 473 | 1138 | 1349 | 864 | −93 | 3 | 48 | | C |
| ATOM | 705 | O | ILE | A | 473 | −10.628 | −22.382 | 0.407 | 1.00 | 9.34 | | | O |
| ANISOU | 705 | O | ILE | A | 473 | 1200 | 1424 | 925 | −47 | 21 | −2 | | O |
| ATOM | 706 | N | THR | A | 474 | −12.647 | −21.377 | 0.564 | 1.00 | 10.23 | | | N |
| ANISOU | 706 | N | THR | A | 474 | 1363 | 1450 | 1073 | −96 | −13 | 47 | | N |
| ATOM | 707 | CA | THR | A | 474 | −13.173 | −22.383 | 1.484 | 1.00 | 12.74 | | | C |
| ANISOU | 707 | CA | THR | A | 474 | 1730 | 1692 | 1418 | −60 | −7 | −9 | | C |
| ATOM | 708 | CB | THR | A | 474 | −13.136 | −21.913 | 2.950 | 1.00 | 13.06 | | | C |
| ANISOU | 708 | CB | THR | A | 474 | 1796 | 1658 | 1510 | −48 | −27 | 7 | | C |
| ATOM | 709 | OG1 | THR | A | 474 | −14.170 | −20.937 | 3.168 | 1.00 | 13.99 | | | O |
| ANISOU | 709 | OG1 | THR | A | 474 | 1926 | 1738 | 1650 | −71 | −48 | 40 | | O |
| ATOM | 710 | CG2 | THR | A | 474 | −11.783 | −21.308 | 3.295 | 1.00 | 11.10 | | | C |
| ANISOU | 710 | CG2 | THR | A | 474 | 1512 | 1438 | 1268 | −53 | −37 | 29 | | C |
| ATOM | 711 | C | THR | A | 474 | −14.616 | −22.749 | 1.140 | 1.00 | 16.76 | | | C |
| ANISOU | 711 | C | THR | A | 474 | 2263 | 2186 | 1919 | −75 | −8 | −28 | | C |
| ATOM | 712 | O | THR | A | 474 | −15.294 | −22.083 | 0.354 | 1.00 | 11.21 | | | O |
| ANISOU | 712 | O | THR | A | 474 | 1538 | 1528 | 1191 | −105 | −20 | 7 | | O |
| ATOM | 713 | N | GLU | A | 475 | −15.095 | −23.815 | 1.778 | 1.00 | 11.78 | | | N |
| ANISOU | 713 | N | GLU | A | 475 | 1674 | 1494 | 1309 | −57 | 2 | −76 | | N |
| ATOM | 714 | CA | GLU | A | 475 | −16.529 | −24.073 | 1.801 | 1.00 | 14.85 | | | C |
| ANISOU | 714 | CA | GLU | A | 475 | 2080 | 1862 | 1700 | −84 | −2 | −86 | | C |
| ATOM | 715 | CB | GLU | A | 475 | −16.830 | −25.311 | 2.659 | 1.00 | 20.29 | | | C |
| ANISOU | 715 | CB | GLU | A | 475 | 2817 | 2472 | 2422 | −73 | 14 | −125 | | C |
| ATOM | 716 | CG | GLU | A | 475 | −17.258 | −25.050 | 4.103 | 1.00 | 18.46 | | | C |
| ANISOU | 716 | CG | GLU | A | 475 | 2604 | 2185 | 2224 | −61 | 6 | −87 | | C |
| ATOM | 717 | CD | GLU | A | 475 | −17.503 | −26.337 | 4.899 | 1.00 | 17.44 | | | C |
| ANISOU | 717 | CD | GLU | A | 475 | 2520 | 1984 | 2123 | −55 | 25 | −103 | | C |
| ATOM | 718 | OE1 | GLU | A | 475 | −16.522 | −26.950 | 5.375 | 1.00 | 16.98 | | | O |
| ANISOU | 718 | OE1 | GLU | A | 475 | 2483 | 1887 | 2081 | −11 | 36 | −103 | | O |
| ATOM | 719 | OE2 | GLU | A | 475 | −18.673 | −26.755 | 5.038 | 1.00 | 19.67 | | | O |
| ANISOU | 719 | OE2 | GLU | A | 475 | 2811 | 2252 | 2411 | −96 | 30 | −105 | | O |
| ATOM | 720 | C | GLU | A | 475 | −17.276 | −22.837 | 2.317 | 1.00 | 11.54 | | | C |
| ANISOU | 720 | C | GLU | A | 475 | 1651 | 1434 | 1301 | −91 | −26 | −28 | | C |
| ATOM | 721 | O | GLU | A | 475 | −16.757 | −22.050 | 3.115 | 1.00 | 8.70 | | | O |
| ANISOU | 721 | O | GLU | A | 475 | 1294 | 1041 | 969 | −73 | −37 | 1 | | O |
| ATOM | 722 | N | TYR | A | 476 | −18.511 | −22.673 | 1.856 | 1.00 | 11.09 | | | N |
| ANISOU | 722 | N | TYR | A | 476 | 1578 | 1409 | 1226 | −115 | −35 | −20 | | N |
| ATOM | 723 | CA | TYR | A | 476 | −19.304 | −21.473 | 2.109 | 1.00 | 13.06 | | | C |
| ANISOU | 723 | CA | TYR | A | 476 | 1811 | 1660 | 1492 | −106 | −56 | 34 | | C |
| ATOM | 724 | CB | TYR | A | 476 | −20.112 | −21.111 | 0.854 | 1.00 | 8.64 | | | C |
| ANISOU | 724 | CB | TYR | A | 476 | 1206 | 1188 | 887 | −126 | −70 | 63 | | C |
| ATOM | 725 | CG | TYR | A | 476 | −21.029 | −19.922 | 1.054 | 1.00 | 10.78 | | | C |
| ANISOU | 725 | CG | TYR | A | 476 | 1457 | 1458 | 1181 | −98 | −92 | 125 | | C |
| ATOM | 726 | CD1 | TYR | A | 476 | −20.537 | −18.718 | 1.551 | 1.00 | 15.22 | | | C |
| ANISOU | 726 | CD1 | TYR | A | 476 | 2036 | 1954 | 1794 | −68 | −104 | 171 | | C |
| ATOM | 727 | CE1 | TYR | A | 476 | −21.378 | −17.632 | 1.738 | 1.00 | 14.93 | | | C |
| ANISOU | 727 | CE1 | TYR | A | 476 | 1987 | 1896 | 1788 | −28 | −122 | 219 | | C |
| ATOM | 728 | CZ | TYR | A | 476 | −22.713 | −17.744 | 1.432 | 1.00 | 15.72 | | | C |
| ANISOU | 728 | CZ | TYR | A | 476 | 2048 | 2065 | 1861 | −13 | −128 | 229 | | C |
| ATOM | 729 | OH | TYR | A | 476 | −23.551 | −16.672 | 1.602 | 1.00 | 21.80 | | | O |
| ANISOU | 729 | OH | TYR | A | 476 | 2801 | 2819 | 2664 | 45 | −145 | 278 | | O |
| ATOM | 730 | CE2 | TYR | A | 476 | −23.226 | −18.921 | 0.938 | 1.00 | 15.56 | | | C |
| ANISOU | 730 | CE2 | TYR | A | 476 | 2002 | 2129 | 1783 | −56 | −119 | 188 | | C |
| ATOM | 731 | CD2 | TYR | A | 476 | −22.380 | −20.002 | 0.748 | 1.00 | 14.26 | | | C |
| ANISOU | 731 | CD2 | TYR | A | 476 | 1861 | 1962 | 1595 | −101 | −101 | 131 | | C |
| ATOM | 732 | C | TYR | A | 476 | −20.249 | −21.688 | 3.287 | 1.00 | 12.57 | | | C |
| ANISOU | 732 | C | TYR | A | 476 | 1763 | 1557 | 1455 | −94 | −53 | 22 | | C |
| ATOM | 733 | O | TYR | A | 476 | −20.915 | −22.721 | 3.368 | 1.00 | 10.74 | | | O |
| ANISOU | 733 | O | TYR | A | 476 | 1536 | 1332 | 1212 | −119 | −40 | −12 | | O |
| ATOM | 734 | N | MET | A | 477 | −20.321 | −20.711 | 4.192 | 1.00 | 7.25 | | | N |
| ANISOU | 734 | N | MET | A | 477 | 1096 | 845 | 814 | −60 | −64 | 45 | | N |
| ATOM | 735 | CA | MET | A | 477 | −21.156 | −20.818 | 5.393 | 1.00 | 7.21 | | | C |
| ANISOU | 735 | CA | MET | A | 477 | 1097 | 822 | 820 | −40 | −56 | 32 | | C |
| ATOM | 736 | CB | MET | A | 477 | −20.301 | −20.700 | 6.664 | 1.00 | 13.57 | | | C |
| ANISOU | 736 | CB | MET | A | 477 | 1933 | 1578 | 1645 | −14 | −54 | 15 | | C |
| ATOM | 737 | CG | MET | A | 477 | −19.190 | −21.744 | 6.732 | 1.00 | 13.10 | | | C |
| ANISOU | 737 | CG | MET | A | 477 | 1897 | 1501 | 1579 | −26 | −42 | −0 | | C |
| ATOM | 738 | SD | MET | A | 477 | −19.795 | −23.413 | 7.091 | 1.00 | 11.77 | | | S |
| ANISOU | 738 | SD | MET | A | 477 | 1748 | 1324 | 1399 | −48 | −14 | −13 | | S |
| ATOM | 739 | CE | MET | A | 477 | −20.350 | −23.167 | 8.761 | 1.00 | 12.11 | | | C |
| ANISOU | 739 | CE | MET | A | 477 | 1792 | 1372 | 1437 | −21 | −8 | 0 | | C |
| ATOM | 740 | C | MET | A | 477 | −22.230 | −19.734 | 5.318 | 1.00 | 9.19 | | | C |
| ANISOU | 740 | C | MET | A | 477 | 1316 | 1096 | 1079 | −9 | −71 | 60 | | C |
| ATOM | 741 | O | MET | A | 477 | −21.983 | −18.570 | 5.658 | 1.00 | 8.84 | | | O |
| ANISOU | 741 | O | MET | A | 477 | 1280 | 1009 | 1069 | 29 | −85 | 73 | | O |
| ATOM | 742 | N | ALA | A | 478 | −23.426 | −20.118 | 4.869 | 1.00 | 9.06 | | | N |
| ANISOU | 742 | N | ALA | A | 478 | 1261 | 1146 | 1035 | −25 | −68 | 65 | | N |
| ATOM | 743 | CA | ALA | A | 478 | −24.398 | −19.136 | 4.398 | 1.00 | 11.49 | | | C |
| ANISOU | 743 | CA | ALA | A | 478 | 1525 | 1499 | 1343 | 12 | −86 | 105 | | C |
| ATOM | 744 | CB | ALA | A | 478 | −25.569 | −19.841 | 3.715 | 1.00 | 11.52 | | | C |
| ANISOU | 744 | CB | ALA | A | 478 | 1472 | 1606 | 1299 | −26 | −86 | 106 | | C |
| ATOM | 745 | C | ALA | A | 478 | −24.949 | −18.234 | 5.493 | 1.00 | 11.55 | | | C |
| ANISOU | 745 | C | ALA | A | 478 | 1530 | 1479 | 1382 | 81 | −85 | 101 | | C |
| ATOM | 746 | O | ALA | A | 478 | −25.433 | −17.140 | 5.183 | 1.00 | 11.86 | | | O |
| ANISOU | 746 | O | ALA | A | 478 | 1548 | 1517 | 1443 | 137 | −102 | 137 | | O |
| ATOM | 747 | N | ASN | A | 479 | −24.935 | −18.661 | 6.752 | 1.00 | 9.87 | | | N |
| ANISOU | 747 | N | ASN | A | 479 | 1334 | 1250 | 1167 | 87 | −65 | 60 | | N |
| ATOM | 748 | CA | ASN | A | 479 | −25.523 | −17.848 | 7.806 | 1.00 | 9.12 | | | C |
| ANISOU | 748 | CA | ASN | A | 479 | 1229 | 1151 | 1086 | 157 | −59 | 39 | | C |
| ATOM | 749 | CB | ASN | A | 479 | −26.343 | −18.737 | 8.739 | 1.00 | 10.62 | | | C |
| ANISOU | 749 | CB | ASN | A | 479 | 1389 | 1413 | 1232 | 143 | −30 | 19 | | C |
| ATOM | 750 | CG | ASN | A | 479 | −27.747 | −18.982 | 8.180 | 1.00 | 16.39 | | | C |
| ANISOU | 750 | CG | ASN | A | 479 | 2044 | 2248 | 1933 | 134 | −25 | 45 | | C |
| ATOM | 751 | OD1 | ASN | A | 479 | −28.356 | −18.070 | 7.640 | 1.00 | 26.49 | | | O |
| ANISOU | 751 | OD1 | ASN | A | 479 | 3287 | 3553 | 3225 | 191 | −42 | 67 | | O |
| ATOM | 752 | ND2 | ASN | A | 479 | −28.236 | −20.209 | 8.273 | 1.00 | 12.47 | | | N |
| ANISOU | 752 | ND2 | ASN | A | 479 | 1525 | 1812 | 1403 | 59 | −5 | 48 | | N |
| ATOM | 753 | C | ASN | A | 479 | −24.494 | −17.009 | 8.561 | 1.00 | 9.84 | | | C |
| ANISOU | 753 | C | ASN | A | 479 | 1372 | 1150 | 1216 | 190 | −69 | 6 | | C |
| ATOM | 754 | O | ASN | A | 479 | −24.851 | −16.345 | 9.541 | 1.00 | 11.81 | | | O |
| ANISOU | 754 | O | ASN | A | 479 | 1621 | 1391 | 1475 | 250 | −64 | −35 | | O |
| ATOM | 755 | N | GLY | A | 480 | −23.257 | −16.955 | 8.078 | 1.00 | 8.93 | | | N |
| ANISOU | 755 | N | GLY | A | 480 | 1294 | 977 | 1123 | 153 | −84 | 16 | | N |
| ATOM | 756 | CA | GLY | A | 480 | −22.306 | −15.975 | 8.582 | 1.00 | 11.03 | | | C |
| ANISOU | 756 | CA | GLY | A | 480 | 1599 | 1158 | 1434 | 170 | −101 | −11 | | C |
| ATOM | 757 | C | GLY | A | 480 | −21.697 | −16.388 | 9.909 | 1.00 | 8.29 | | | C |
| ANISOU | 757 | C | GLY | A | 480 | 1272 | 814 | 1062 | 167 | −92 | −69 | | C |
| ATOM | 758 | O | GLY | A | 480 | −21.731 | −17.543 | 10.301 | 1.00 | 9.94 | | | O |
| ANISOU | 758 | O | GLY | A | 480 | 1474 | 1077 | 1225 | 144 | −72 | −68 | | O |
| ATOM | 759 | N | CYS | A | 481 | −21.148 | −15.413 | 10.619 | 1.00 | 8.69 | | | N |
| ANISOU | 759 | N | CYS | A | 481 | 1348 | 807 | 1146 | 188 | −109 | −118 | | N |
| ATOM | 760 | CA | CYS | A | 481 | −20.407 | −15.733 | 11.826 | 1.00 | 13.10 | | | C |
| ANISOU | 760 | CA | CYS | A | 481 | 1919 | 1388 | 1670 | 181 | −109 | −173 | | C |
| ATOM | 761 | CB | CYS | A | 481 | −19.439 | −14.611 | 12.180 | 1.00 | 13.16 | | | C |
| ANISOU | 761 | CB | CYS | A | 481 | 1956 | 1320 | 1724 | 171 | −139 | −225 | | C |
| ATOM | 762 | SG | CYS | A | 481 | −20.231 | −13.078 | 12.605 | 1.00 | 11.53 | | | S |
| ANISOU | 762 | SG | CYS | A | 481 | 1769 | 1035 | 1578 | 238 | −150 | −292 | | S |
| ATOM | 763 | C | CYS | A | 481 | −21.344 | −15.997 | 13.001 | 1.00 | 11.20 | | | C |
| ANISOU | 763 | C | CYS | A | 481 | 1660 | 1221 | 1376 | 229 | −85 | −214 | | C |
| ATOM | 764 | O | CYS | A | 481 | −22.410 | −15.383 | 13.131 | 1.00 | 9.45 | | | O |
| ANISOU | 764 | O | CYS | A | 481 | 1421 | 1006 | 1164 | 284 | −77 | −236 | | O |
| ATOM | 765 | N | LEU | A | 482 | −20.939 | −16.938 | 13.849 | 1.00 | 7.01 | | | N |
| ANISOU | 765 | N | LEU | A | 482 | 1067 | 661 | 936 | −86 | 42 | 263 | | N |
| ATOM | 766 | CA | LEU | A | 482 | −21.761 | −17.325 | 14.990 | 1.00 | 6.50 | | | C |
| ANISOU | 766 | CA | LEU | A | 482 | 1019 | 582 | 867 | −90 | 10 | 176 | | C |
| ATOM | 767 | CB | LEU | A | 482 | −21.023 | −18.370 | 15.821 | 1.00 | 7.41 | | | C |
| ANISOU | 767 | CB | LEU | A | 482 | 1151 | 708 | 957 | −121 | −6 | 100 | | C |
| ATOM | 768 | CG | LEU | A | 482 | −21.752 | −18.894 | 17.048 | 1.00 | 13.04 | | | C |
| ANISOU | 768 | CG | LEU | A | 482 | 1873 | 1432 | 1651 | −119 | −24 | 43 | | C |
| ATOM | 769 | CD1 | LEU | A | 482 | −23.083 | −19.567 | 16.660 | 1.00 | 7.57 | | | C |
| ANISOU | 769 | CD1 | LEU | A | 482 | 1175 | 786 | 915 | −111 | −32 | 55 | | C |
| ATOM | 770 | CD2 | LEU | A | 482 | −20.805 | −19.848 | 17.835 | 1.00 | 10.48 | | | C |
| ANISOU | 770 | CD2 | LEU | A | 482 | 1554 | 1123 | 1303 | −132 | −31 | 8 | | C |
| ATOM | 771 | C | LEU | A | 482 | −22.123 | −16.127 | 15.866 | 1.00 | 7.67 | | | C |
| ANISOU | 771 | C | LEU | A | 482 | 1157 | 639 | 1117 | −78 | 15 | 154 | | C |
| ATOM | 772 | O | LEU | A | 482 | −23.243 | −16.038 | 16.391 | 1.00 | 7.84 | | | O |
| ANISOU | 772 | O | LEU | A | 482 | 1178 | 672 | 1130 | −53 | 2 | 125 | | O |
| ATOM | 773 | N | LEU | A | 483 | −21.182 | −15.218 | 16.069 | 1.00 | 7.40 | | | N |
| ANISOU | 773 | N | LEU | A | 483 | 1105 | 513 | 1194 | −98 | 36 | 150 | | N |
| ATOM | 774 | CA | LEU | A | 483 | −21.415 | −14.096 | 16.972 | 1.00 | 12.81 | | | C |
| ANISOU | 774 | CA | LEU | A | 483 | 1767 | 1096 | 2004 | −89 | 37 | 82 | | C |
| ATOM | 775 | CB | LEU | A | 483 | −20.172 | −13.202 | 16.984 | 1.00 | 13.44 | | | C |
| ANISOU | 775 | CB | LEU | A | 483 | 1807 | 1061 | 2238 | −130 | 62 | 70 | | C |
| ATOM | 776 | CG | LEU | A | 483 | −20.293 | −11.974 | 17.865 | 1.00 | 14.42 | | | C |
| ANISOU | 776 | CG | LEU | A | 483 | 1890 | 1078 | 2509 | −124 | 60 | −38 | | C |
| ATOM | 777 | CD1 | LEU | A | 483 | −20.485 | −12.457 | 19.310 | 1.00 | 16.00 | | | C |
| ANISOU | 777 | CD1 | LEU | A | 483 | 2091 | 1343 | 2643 | −118 | 10 | −217 | | C |
| ATOM | 778 | CD2 | LEU | A | 483 | −19.062 | −11.095 | 17.742 | 1.00 | 14.10 | | | C |
| ANISOU | 778 | CD2 | LEU | A | 483 | 1793 | 978 | 2585 | −164 | 82 | −45 | | C |
| ATOM | 779 | C | LEU | A | 483 | −22.658 | −13.297 | 16.574 | 1.00 | 10.70 | | | C |
| ANISOU | 779 | C | LEU | A | 483 | 1490 | 790 | 1785 | −34 | 51 | 148 | | C |
| ATOM | 780 | O | LEU | A | 483 | −23.553 | −13.052 | 17.399 | 1.00 | 9.59 | | | O |
| ANISOU | 780 | O | LEU | A | 483 | 1344 | 645 | 1654 | −3 | 36 | 70 | | O |
| ATOM | 781 | N | ASN | A | 484 | −22.725 | −12.852 | 15.312 | 1.00 | 9.19 | | | N |
| ANISOU | 781 | N | ASN | A | 484 | 1287 | 585 | 1619 | −8 | 85 | 301 | | N |
| ATOM | 782 | C | ASN | A | 484 | −25.136 | −12.921 | 14.859 | 1.00 | 12.34 | | | C |
| ANISOU | 782 | C | ASN | A | 484 | 1676 | 1099 | 1911 | 97 | 58 | 363 | | C |
| ATOM | 783 | O | ASN | A | 484 | −26.244 | −12.422 | 15.099 | 1.00 | 12.11 | | | O |
| ANISOU | 783 | O | ASN | A | 484 | 1627 | 1059 | 1914 | 152 | 53 | 360 | | O |
| ATOM | 784 | CA | ASN | A | 484 | −23.878 | −12.070 | 14.883 | 1.00 | 9.96 | | | C |
| ANISOU | 784 | CA | ASN | A | 484 | 1365 | 656 | 1762 | 63 | 98 | 387 | | C |
| ATOM | 785 | CB | ASN | A | 484 | −23.628 | −11.469 | 13.495 | 1.00 | 10.97 | | | C |
| ANISOU | 785 | CB | ASN | A | 484 | 1467 | 793 | 1910 | 97 | 143 | 576 | | C |
| ATOM | 786 | CG | ASN | A | 484 | −22.550 | −10.415 | 13.507 | 1.00 | 20.33 | | | C |
| ANISOU | 786 | CG | ASN | A | 484 | 2616 | 1869 | 3240 | 58 | 184 | 575 | | C |
| ATOM | 787 | OD1 | ASN | A | 484 | −22.189 | −9.895 | 14.559 | 1.00 | 22.92 | | | O |
| ANISOU | 787 | OD1 | ASN | A | 484 | 2933 | 2087 | 3690 | 22 | 177 | 444 | | O |
| ATOM | 788 | ND2 | ASN | A | 484 | −22.042 | −10.077 | 12.333 | 1.00 | 25.94 | | | N |
| ANISOU | 788 | ND2 | ASN | A | 484 | 3294 | 2626 | 3936 | 72 | 227 | 717 | | N |
| ATOM | 789 | N | TYR | A | 485 | −24.984 | −14.203 | 14.563 | 1.00 | 10.92 | | | N |
| ANISOU | 789 | N | TYR | A | 485 | 1517 | 1045 | 1588 | 65 | 33 | 338 | | N |
| ATOM | 790 | CA | TYR | A | 485 | −26.124 | −15.113 | 14.552 | 1.00 | 8.05 | | | C |
| ANISOU | 790 | CA | TYR | A | 485 | 1146 | 799 | 1113 | 77 | −3 | 298 | | C |
| ATOM | 791 | CB | TYR | A | 485 | −25.626 | −16.454 | 14.030 | 1.00 | 8.40 | | | C |
| ANISOU | 791 | CB | TYR | A | 485 | 1203 | 938 | 1050 | 34 | −21 | 269 | | C |
| ATOM | 792 | CG | TYR | A | 485 | −26.626 | −17.566 | 13.799 | 1.00 | 10.14 | | | C |
| ANISOU | 792 | CG | TYR | A | 485 | 1401 | 1268 | 1185 | 27 | −56 | 215 | | C |
| ATOM | 793 | CD1 | TYR | A | 485 | −27.462 | −17.547 | 12.690 | 1.00 | 14.20 | | | C |
| ANISOU | 793 | CD1 | TYR | A | 485 | 1868 | 1899 | 1629 | 74 | −79 | 254 | | C |
| ATOM | 794 | CE1 | TYR | A | 485 | −28.340 | −18.591 | 12.439 | 1.00 | 20.48 | | | C |
| ANISOU | 794 | CE1 | TYR | A | 485 | 2620 | 2792 | 2368 | 56 | −117 | 174 | | C |
| ATOM | 795 | CZ | TYR | A | 485 | −28.342 | −19.680 | 13.288 | 1.00 | 18.50 | | | C |
| ANISOU | 795 | CZ | TYR | A | 485 | 2382 | 2493 | 2154 | −13 | −117 | 83 | | C |
| ATOM | 796 | OH | TYR | A | 485 | −29.179 | −20.730 | 13.054 | 1.00 | 24.67 | | | O |
| ANISOU | 796 | OH | TYR | A | 485 | 3109 | 3338 | 2928 | −45 | −145 | 1 | | O |
| ATOM | 797 | CE2 | TYR | A | 485 | −27.479 | −19.739 | 14.368 | 1.00 | 22.53 | | | C |
| ANISOU | 797 | CE2 | TYR | A | 485 | 2945 | 2896 | 2718 | −47 | −87 | 78 | | C |
| ATOM | 798 | CD2 | TYR | A | 485 | −26.620 | −18.690 | 14.609 | 1.00 | 12.51 | | | C |
| ANISOU | 798 | CD2 | TYR | A | 485 | 1713 | 1559 | 1481 | −26 | −64 | 130 | | C |
| ATOM | 799 | C | TYR | A | 485 | −26.741 | −15.239 | 15.940 | 1.00 | 11.80 | | | C |
| ANISOU | 799 | C | TYR | A | 485 | 1623 | 1251 | 1610 | 68 | −13 | 190 | | C |
| ATOM | 800 | O | TYR | A | 485 | −27.971 | −15.224 | 16.097 | 1.00 | 11.02 | | | O |
| ANISOU | 800 | O | TYR | A | 485 | 1494 | 1200 | 1492 | 103 | −23 | 181 | | O |
| ATOM | 801 | N | LEU | A | 486 | −25.897 | −15.368 | 16.963 | 1.00 | 7.37 | | | N |
| ANISOU | 801 | N | LEU | A | 486 | 1083 | 643 | 1075 | 29 | −9 | 110 | | N |
| ATOM | 802 | CA | LEU | A | 486 | −26.384 | −15.447 | 18.334 | 1.00 | 7.34 | | | C |
| ANISOU | 802 | CA | LEU | A | 486 | 1070 | 656 | 1063 | 37 | −13 | 14 | | C |
| ATOM | 803 | CB | LEU | A | 486 | −25.213 | −15.695 | 19.285 | 1.00 | 7.13 | | | C |
| ANISOU | 803 | CB | LEU | A | 486 | 1059 | 621 | 1032 | 3 | −17 | −63 | | C |
| ATOM | 804 | CG | LEU | A | 486 | −24.495 | −17.042 | 19.274 | 1.00 | 11.14 | | | C |
| ANISOU | 804 | CG | LEU | A | 486 | 1589 | 1185 | 1457 | −39 | −25 | −46 | | C |
| ATOM | 805 | CD1 | LEU | A | 486 | −23.141 | −16.854 | 19.956 | 1.00 | 6.57 | | | C |
| ANISOU | 805 | CD1 | LEU | A | 486 | 1011 | 587 | 899 | −56 | −33 | −109 | | C |
| ATOM | 806 | CD2 | LEU | A | 486 | −25.339 | −18.100 | 19.995 | 1.00 | 7.68 | | | C |
| ANISOU | 806 | CD2 | LEU | A | 486 | 1142 | 833 | 943 | −36 | −20 | −46 | | C |
| ATOM | 807 | C | LEU | A | 486 | −27.113 | −14.183 | 18.753 | 1.00 | 12.45 | | | C |
| ANISOU | 807 | C | LEU | A | 486 | 1686 | 1243 | 1801 | 95 | −2 | −19 | | C |
| ATOM | 808 | O | LEU | A | 486 | −28.062 | −14.247 | 19.543 | 1.00 | 10.79 | | | O |
| ANISOU | 808 | O | LEU | A | 486 | 1451 | 1090 | 1558 | 126 | −1 | −74 | | O |
| ATOM | 809 | N | ARG | A | 487 | −26.683 | −13.030 | 18.246 | 1.00 | 9.33 | | | N |
| ANISOU | 809 | N | ARG | A | 487 | 1283 | 727 | 1534 | 114 | 14 | 17 | | N |
| ATOM | 810 | CA | ARG | A | 487 | −27.268 | −11.760 | 18.643 | 1.00 | 9.97 | | | C |
| ANISOU | 810 | CA | ARG | A | 487 | 1330 | 710 | 1750 | 174 | 29 | −25 | | C |
| ATOM | 811 | CB | ARG | A | 487 | −26.286 | −10.632 | 18.351 | 1.00 | 10.90 | | | C |
| ANISOU | 811 | CB | ARG | A | 487 | 1435 | 651 | 2055 | 162 | 55 | −9 | | C |
| ATOM | 812 | CG | ARG | A | 487 | −25.040 | −10.699 | 19.249 | 1.00 | 10.83 | | | C |
| ANISOU | 812 | CG | ARG | A | 487 | 1425 | 612 | 2077 | 99 | 42 | −152 | | C |
| ATOM | 813 | CD | ARG | A | 487 | −23.990 | −9.661 | 18.827 | 1.00 | 14.44 | | | C |
| ANISOU | 813 | CD | ARG | A | 487 | 1854 | 934 | 2699 | 62 | 70 | −120 | | C |
| ATOM | 814 | NE | ARG | A | 487 | −22.904 | −9.608 | 19.801 | 1.00 | 13.59 | | | N |
| ANISOU | 814 | NE | ARG | A | 487 | 1720 | 844 | 2600 | 9 | 46 | −278 | | N |
| ATOM | 815 | CZ | ARG | A | 487 | −21.808 | −8.869 | 19.672 | 1.00 | 19.96 | | | C |
| ANISOU | 815 | Cz | ARG | A | 487 | 2483 | 1585 | 3516 | −37 | 61 | −283 | | C |
| ATOM | 816 | NH1 | ARG | A | 487 | −21.634 | −8.118 | 18.589 | 1.00 | 18.13 | | | N |
| ANISOU | 816 | NH1 | ARG | A | 487 | 2233 | 1261 | 3393 | −40 | 108 | −132 | | N |
| ATOM | 817 | NH2 | ARG | A | 487 | −20.878 | −8.893 | 20.628 | 1.00 | 19.97 | | | N |
| ANISOU | 817 | NH2 | ARG | A | 487 | 2447 | 1628 | 3514 | −73 | 30 | −435 | | N |
| ATOM | 818 | C | ARG | A | 487 | −28.609 | −11.459 | 17.977 | 1.00 | 12.23 | | | C |
| ANISOU | 818 | C | ARG | A | 487 | 1588 | 1026 | 2033 | 247 | 34 | 68 | | C |
| ATOM | 819 | O | ARG | A | 487 | −29.278 | −10.515 | 18.403 | 1.00 | 11.60 | | | O |
| ANISOU | 819 | O | ARG | A | 487 | 1472 | 875 | 2059 | 311 | 46 | 24 | | O |
| ATOM | 820 | N | GLU | A | 488 | −29.007 | −12.206 | 16.949 | 1.00 | 12.66 | | | N |
| ANISOU | 820 | N | GLU | A | 488 | 1645 | 1189 | 1975 | 245 | 20 | 178 | | N |
| ATOM | 821 | CA | GLU | A | 488 | −30.317 | −12.008 | 16.322 | 1.00 | 13.09 | | | C |
| ANISOU | 821 | CA | GLU | A | 488 | 1656 | 1314 | 2005 | 320 | 11 | 255 | | C |
| ATOM | 822 | CB | GLU | A | 488 | −30.325 | −12.572 | 14.905 | 1.00 | 16.34 | | | C |
| ANISOU | 822 | CB | GLU | A | 488 | 2061 | 1838 | 2309 | 323 | −8 | 378 | | C |
| ATOM | 823 | CG | GLU | A | 488 | −29.420 | −11.801 | 13.946 | 1.00 | 21.49 | | | C |
| ANISOU | 823 | CG | GLU | A | 488 | 2722 | 2415 | 3027 | 346 | 24 | 520 | | C |
| ATOM | 824 | CD | GLU | A | 488 | −29.194 | −12.507 | 12.598 | 1.00 | 46.30 | | | C |
| ANISOU | 824 | CD | GLU | A | 488 | 5855 | 5717 | 6020 | 349 | 7 | 617 | | C |
| ATOM | 825 | OE1 | GLU | A | 488 | −28.940 | −13.739 | 12.565 | 1.00 | 40.62 | | | O |
| ANISOU | 825 | OE1 | GLU | A | 488 | 5154 | 5096 | 5184 | 283 | −23 | 529 | | O |
| ATOM | 826 | OE2 | GLU | A | 488 | −29.262 | −11.812 | 11.558 | 1.00 | 60.61 | | | O |
| ANISOU | 826 | OE2 | GLU | A | 488 | 7635 | 7561 | 7834 | 427 | 27 | 784 | | O |
| ATOM | 827 | C | GLU | A | 488 | −31.364 | −12.682 | 17.199 | 1.00 | 10.22 | | | C |
| ANISOU | 827 | C | GLU | A | 488 | 1265 | 1056 | 1564 | 318 | −4 | 156 | | C |
| ATOM | 828 | O | GLU | A | 488 | −31.519 | −13.906 | 17.178 | 1.00 | 10.90 | | | O |
| ANISOU | 828 | O | GLU | A | 488 | 1352 | 1252 | 1538 | 262 | −22 | 137 | | O |
| ATOM | 829 | N | MET | A | 489 | −32.102 | −11.884 | 17.967 | 1.00 | 10.98 | | | N |
| ANISOU | 829 | N | MET | A | 489 | 1324 | 1113 | 1734 | 382 | 12 | 94 | | N |
| ATOM | 830 | CA | MET | A | 489 | −33.042 | −12.465 | 18.920 | 1.00 | 11.51 | | | C |
| ANISOU | 830 | CA | MET | A | 489 | 1354 | 1294 | 1727 | 383 | 14 | 6 | | C |
| ATOM | 831 | CB | MET | A | 489 | −33.564 | −11.371 | 19.851 | 1.00 | 15.02 | | | C |
| ANISOU | 831 | CB | MET | A | 489 | 1759 | 1682 | 2268 | 465 | 37 | −91 | | C |
| ATOM | 832 | CG | MET | A | 489 | −32.477 | −10.740 | 20.740 | 1.00 | 24.54 | | | C |
| ANISOU | 832 | CG | MET | A | 489 | 2994 | 2775 | 3556 | 452 | 49 | −216 | | C |
| ATOM | 833 | SD | MET | A | 489 | −31.675 | −11.935 | 21.862 | 1.00 | 22.85 | | | S |
| ANISOU | 833 | SD | MET | A | 489 | 2810 | 2685 | 3188 | 369 | 45 | −308 | | S |
| ATOM | 834 | CE | MET | A | 489 | −33.039 | −12.256 | 22.990 | 1.00 | 29.76 | | | C |
| ANISOU | 834 | CE | MET | A | 489 | 3615 | 3735 | 3959 | 426 | 72 | −379 | | C |
| ATOM | 835 | C | MET | A | 489 | −34.206 | −13.190 | 18.236 | 1.00 | 15.63 | | | C |
| ANISOU | 835 | C | MET | A | 489 | 1821 | 1954 | 2165 | 390 | −7 | 68 | | C |
| ATOM | 836 | O | MET | A | 489 | −34.854 | −14.035 | 18.866 | 1.00 | 12.51 | | | O |
| ANISOU | 836 | O | MET | A | 489 | 1389 | 1660 | 1702 | 356 | 0 | 21 | | O |
| ATOM | 837 | N | ARG | A | 490 | −34.492 | −12.877 | 16.974 | 1.00 | 14.83 | | | N |
| ANISOU | 837 | N | ARG | A | 490 | 1698 | 1870 | 2068 | 434 | −31 | 174 | | N |
| ATOM | 838 | CA | ARG | A | 490 | −35.618 | −13.508 | 16.296 | 1.00 | 15.51 | | | C |
| ANISOU | 838 | CA | ARG | A | 490 | 1710 | 2110 | 2073 | 446 | −64 | 204 | | C |
| ATOM | 839 | CB | ARG | A | 490 | −35.721 | −12.975 | 14.869 | 1.00 | 12.39 | | | C |
| ANISOU | 839 | CB | ARG | A | 490 | 1289 | 1755 | 1662 | 521 | −93 | 332 | | C |
| ATOM | 840 | CG | ARG | A | 490 | −34.476 | −13.234 | 14.019 | 1.00 | 11.94 | | | C |
| ANISOU | 840 | CG | ARG | A | 490 | 1295 | 1679 | 1565 | 478 | −99 | 394 | | C |
| ATOM | 841 | CD | ARG | A | 490 | −34.612 | −12.650 | 12.589 | 1.00 | 16.04 | | | C |
| ANISOU | 841 | CD | ARG | A | 490 | 1773 | 2280 | 2040 | 575 | −117 | 551 | | C |
| ATOM | 842 | NE | ARG | A | 490 | −33.338 | −12.676 | 11.857 | 1.00 | 13.98 | | | N |
| ANISOU | 842 | NE | ARG | A | 490 | 1566 | 1995 | 1750 | 549 | −101 | 630 | | N |
| ATOM | 843 | CZ | ARG | A | 490 | −32.885 | −13.721 | 11.177 | 1.00 | 13.09 | | | C |
| ANISOU | 843 | CZ | ARG | A | 490 | 1458 | 2008 | 1508 | 493 | −131 | 593 | | C |
| ATOM | 844 | NH1 | ARG | A | 490 | −33.603 | −14.833 | 11.117 | 1.00 | 16.68 | | | N |
| ANISOU | 844 | NH1 | ARG | A | 490 | 1865 | 2599 | 1873 | 448 | −182 | 474 | | N |
| ATOM | 845 | NH2 | ARG | A | 490 | −31.705 | −13.665 | 10.554 | 1.00 | 15.28 | | | N |
| ANISOU | 845 | NH2 | ARG | A | 490 | 1777 | 2269 | 1761 | 479 | −107 | 667 | | N |
| ATOM | 846 | C | ARG | A | 490 | −35.499 | −15.032 | 16.302 | 1.00 | 16.72 | | | C |
| ANISOU | 846 | C | ARG | A | 490 | 1864 | 2350 | 2140 | 336 | −80 | 152 | | C |
| ATOM | 847 | O | ARG | A | 490 | −36.513 | −15.732 | 16.215 | 1.00 | 13.16 | | | O |
| ANISOU | 847 | O | ARG | A | 490 | 1336 | 2012 | 1654 | 316 | −97 | 123 | | O |
| ATOM | 848 | N | HIS | A | 491 | −34.276 | −15.568 | 16.415 | 1.00 | 10.55 | | | N |
| ANISOU | 848 | N | HIS | A | 491 | 1158 | 1506 | 1344 | 263 | −73 | 137 | | N |
| ATOM | 849 | CA | HIS | A | 491 | −34.109 | −17.018 | 16.328 | 1.00 | 9.95 | | | C |
| ANISOU | 849 | CA | HIS | A | 491 | 1080 | 1483 | 1218 | 167 | −84 | 96 | | C |
| ATOM | 850 | CB | HIS | A | 491 | −32.633 | −17.416 | 16.297 | 1.00 | 14.17 | | | C |
| ANISOU | 850 | CB | HIS | A | 491 | 1698 | 1941 | 1744 | 113 | −77 | 95 | | C |
| ATOM | 851 | CG | HIS | A | 491 | −31.910 | −16.983 | 15.059 | 1.00 | 13.29 | | | C |
| ANISOU | 851 | CG | HIS | A | 491 | 1612 | 1828 | 1608 | 141 | −98 | 155 | | C |
| ATOM | 852 | ND1 | HIS | A | 491 | −32.246 | −17.441 | 13.802 | 1.00 | 16.49 | | | N |
| ANISOU | 852 | ND1 | HIS | A | 491 | 1972 | 2351 | 1943 | 151 | −139 | 166 | | N |
| ATOM | 853 | CE1 | HIS | A | 491 | −31.445 | −16.893 | 12.906 | 1.00 | 14.92 | | | C |
| ANISOU | 853 | CE1 | HIS | A | 491 | 1800 | 2155 | 1714 | 190 | −138 | 244 | | C |
| ATOM | 854 | NE2 | HIS | A | 491 | −30.595 | −16.103 | 13.540 | 1.00 | 20.00 | | | N |
| ANISOU | 854 | NE2 | HIS | A | 491 | 2503 | 2661 | 2436 | 191 | −98 | 281 | | N |
| ATOM | 855 | CD2 | HIS | A | 491 | −30.866 | −16.142 | 14.887 | 1.00 | 15.64 | | | C |
| ANISOU | 855 | CD2 | HIS | A | 491 | 1962 | 2038 | 1942 | 164 | −80 | 210 | | C |
| ATOM | 856 | C | HIS | A | 491 | −34.773 | −17.734 | 17.487 | 1.00 | 9.39 | | | C |
| ANISOU | 856 | C | HIS | A | 491 | 970 | 1439 | 1158 | 123 | −51 | 47 | | C |
| ATOM | 857 | O | HIS | A | 491 | −35.121 | −18.910 | 17.358 | 1.00 | 11.97 | | | O |
| ANISOU | 857 | O | HIS | A | 491 | 1254 | 1810 | 1484 | 52 | −55 | 24 | | O |
| ATOM | 858 | N | ARG | A | 492 | −34.927 | −17.067 | 18.628 | 1.00 | 9.54 | | | N |
| ANISOU | 858 | N | ARG | A | 492 | 993 | 1436 | 1196 | 165 | −12 | 29 | | N |
| ATOM | 859 | CA | ARG | A | 492 | −35.644 | −17.644 | 19.770 | 1.00 | 9.85 | | | C |
| ANISOU | 859 | CA | ARG | A | 492 | 979 | 1539 | 1224 | 144 | 33 | 7 | | C |
| ATOM | 860 | CB | ARG | A | 492 | −37.151 | −17.669 | 19.505 | 1.00 | 12.88 | | | C |
| ANISOU | 860 | CB | ARG | A | 492 | 1253 | 2020 | 1622 | 166 | 30 | 8 | | C |
| ATOM | 861 | CG | ARG | A | 492 | −37.782 | −16.292 | 19.417 | 1.00 | 16.71 | | | C |
| ANISOU | 861 | CG | ARG | A | 492 | 1707 | 2513 | 2128 | 281 | 22 | 1 | | C |
| ATOM | 862 | CD | ARG | A | 492 | −39.187 | −16.422 | 18.884 | 1.00 | 17.14 | | | C |
| ANISOU | 862 | CD | ARG | A | 492 | 1643 | 2680 | 2190 | 303 | 2 | 8 | | C |
| ATOM | 863 | NE | ARG | A | 492 | −40.022 | −15.251 | 19.118 | 1.00 | 16.64 | | | N |
| ANISOU | 863 | NE | ARG | A | 492 | 1525 | 2641 | 2156 | 422 | 12 | 1 | | N |
| ATOM | 864 | CZ | ARG | A | 492 | −39.975 | −14.154 | 18.376 | 1.00 | 18.22 | | | C |
| ANISOU | 864 | Cz | ARG | A | 492 | 1740 | 2792 | 2393 | 520 | −20 | 41 | | C |
| ATOM | 865 | NH1 | ARG | A | 492 | −39.084 | −14.063 | 17.390 | 1.00 | 16.46 | | | N |
| ANISOU | 865 | NH1 | ARG | A | 492 | 1584 | 2508 | 2161 | 509 | −58 | 99 | | N |
| ATOM | 866 | NH2 | ARG | A | 492 | −40.795 | −13.144 | 18.631 | 1.00 | 20.69 | | | N |
| ANISOU | 866 | NH2 | ARG | A | 492 | 1992 | 3113 | 2756 | 635 | −7 | 34 | | N |
| ATOM | 867 | C | ARG | A | 492 | −35.136 | −19.046 | 20.092 | 1.00 | 10.92 | | | C |
| ANISOU | 867 | C | ARG | A | 492 | 1131 | 1669 | 1351 | 49 | 55 | 25 | | C |
| ATOM | 868 | O | ARG | A | 492 | −35.901 | −20.012 | 20.162 | 1.00 | 11.10 | | | O |
| ANISOU | 868 | O | ARG | A | 492 | 1082 | 1736 | 1399 | −7 | 76 | 40 | | O |
| ATOM | 869 | N | PHE | A | 493 | −33.821 | −19.148 | 20.274 | 1.00 | 10.70 | | | N |
| ANISOU | 869 | N | PHE | A | 493 | 1187 | 1572 | 1306 | 31 | 53 | 27 | | N |
| ATOM | 870 | CA | PHE | A | 493 | −33.163 | −20.440 | 20.435 | 1.00 | 10.55 | | | C |
| ANISOU | 870 | CA | PHE | A | 493 | 1190 | 1525 | 1294 | −43 | 69 | 56 | | C |
| ATOM | 871 | CB | PHE | A | 493 | −31.639 | −20.260 | 20.596 | 1.00 | 8.51 | | | C |
| ANISOU | 871 | CB | PHE | A | 493 | 1019 | 1203 | 1010 | −39 | 58 | 50 | | C |
| ATOM | 872 | CG | PHE | A | 493 | −30.930 | −19.770 | 19.344 | 1.00 | 12.43 | | | C |
| ANISOU | 872 | CG | PHE | A | 493 | 1562 | 1643 | 1520 | −36 | 10 | 35 | | C |
| ATOM | 873 | CD1 | PHE | A | 493 | −30.833 | −20.572 | 18.208 | 1.00 | 8.64 | | | C |
| ANISOU | 873 | CD1 | PHE | A | 493 | 1076 | 1156 | 1053 | −81 | −17 | 33 | | C |
| ATOM | 874 | CE1 | PHE | A | 493 | −30.167 | −20.127 | 17.070 | 1.00 | 14.32 | | | C |
| ANISOU | 874 | CE1 | PHE | A | 493 | 1826 | 1862 | 1755 | −65 | −50 | 34 | | C |
| ATOM | 875 | CZ | PHE | A | 493 | −29.570 | −18.874 | 17.058 | 1.00 | 13.65 | | | C |
| ANISOU | 875 | CZ | PHE | A | 493 | 1778 | 1736 | 1671 | −16 | −49 | 58 | | C |
| ATOM | 876 | CE2 | PHE | A | 493 | −29.642 | −18.074 | 18.179 | 1.00 | 14.49 | | | C |
| ANISOU | 876 | CE2 | PHE | A | 493 | 1891 | 1813 | 1801 | 17 | −27 | 41 | | C |
| ATOM | 877 | CD2 | PHE | A | 493 | −30.325 | −18.526 | 19.323 | 1.00 | 9.00 | | | C |
| ANISOU | 877 | CD2 | PHE | A | 493 | 1165 | 1165 | 1091 | 13 | −1 | 19 | | C |
| ATOM | 878 | C | PHE | A | 493 | −33.691 | −21.179 | 21.657 | 1.00 | 11.72 | | | C |
| ANISOU | 878 | C | PHE | A | 493 | 1286 | 1729 | 1436 | −60 | 136 | 104 | | C |
| ATOM | 879 | O | PHE | A | 493 | −33.938 | −20.579 | 22.710 | 1.00 | 11.88 | | | O |
| ANISOU | 879 | O | PHE | A | 493 | 1290 | 1823 | 1400 | −0 | 170 | 101 | | O |
| ATOM | 880 | N | GLN | A | 494 | −33.845 | −22.495 | 21.515 | 1.00 | 9.40 | | | N |
| ANISOU | 880 | N | GLN | A | 494 | 958 | 1405 | 1210 | −136 | 160 | 150 | | N |
| ATOM | 881 | CA | GLN | A | 494 | −33.957 | −23.378 | 22.668 | 1.00 | 13.01 | | | C |
| ANISOU | 881 | CA | GLN | A | 494 | 1377 | 1888 | 1676 | −155 | 238 | 246 | | C |
| ATOM | 882 | CB | GLN | A | 494 | −34.618 | −24.701 | 22.294 | 1.00 | 14.26 | | | C |
| ANISOU | 882 | CB | GLN | A | 494 | 1456 | 1985 | 1975 | −250 | 270 | 287 | | C |
| ATOM | 883 | CG | GLN | A | 494 | −35.951 | −24.623 | 21.581 | 1.00 | 16.71 | | | C |
| ANISOU | 883 | CG | GLN | A | 494 | 1668 | 2330 | 2352 | −285 | 249 | 226 | | C |
| ATOM | 884 | CD | GLN | A | 494 | −36.498 | −26.023 | 21.364 | 1.00 | 21.00 | | | C |
| ANISOU | 884 | CD | GLN | A | 494 | 2116 | 2793 | 3070 | −393 | 288 | 251 | | C |
| ATOM | 885 | OE1 | GLN | A | 494 | −36.828 | −26.722 | 22.322 | 1.00 | 28.08 | | | O |
| ANISOU | 885 | OE1 | GLN | A | 494 | 2955 | 3684 | 4031 | −421 | 381 | 373 | | O |
| ATOM | 886 | NE2 | GLN | A | 494 | −36.527 | −26.461 | 20.112 | 1.00 | 28.53 | | | N |
| ANISOU | 886 | NE2 | GLN | A | 494 | 3047 | 3684 | 4108 | −450 | 220 | 135 | | N |
| ATOM | 887 | C | GLN | A | 494 | −32.568 | −23.682 | 23.214 | 1.00 | 11.96 | | | C |
| ANISOU | 887 | C | GLN | A | 494 | 1323 | 1724 | 1498 | −138 | 245 | 288 | | C |
| ATOM | 888 | O | GLN | A | 494 | −31.577 | −23.664 | 22.480 | 1.00 | 10.33 | | | O |
| ANISOU | 888 | O | GLN | A | 494 | 1184 | 1439 | 1302 | −150 | 194 | 243 | | O |
| ATOM | 889 | N | THR | A | 495 | −32.490 | −23.982 | 24.508 | 1.00 | 12.39 | | | N |
| ANISOU | 889 | N | THR | A | 495 | 1355 | 1863 | 1492 | −101 | 310 | 383 | | N |
| ATOM | 890 | CA | THR | A | 495 | −31.172 | −24.309 | 25.053 | 1.00 | 14.26 | | | C |
| ANISOU | 890 | CA | THR | A | 495 | 1650 | 2096 | 1673 | −72 | 311 | 430 | | C |
| ATOM | 891 | CB | THR | A | 495 | −31.226 | −24.450 | 26.577 | 1.00 | 11.75 | | | C |
| ANISOU | 891 | CB | THR | A | 495 | 1287 | 1938 | 1237 | −0 | 381 | 537 | | C |
| ATOM | 892 | OG1 | THR | A | 495 | −32.195 | −25.442 | 26.951 | 1.00 | 13.18 | | | O |
| ANISOU | 892 | OG1 | THR | A | 495 | 1383 | 2130 | 1495 | −37 | 472 | 686 | | O |
| ATOM | 893 | CG2 | THR | A | 495 | −31.578 | −23.102 | 27.205 | 1.00 | 12.19 | | | C |
| ANISOU | 893 | CG2 | THR | A | 495 | 1329 | 2143 | 1159 | 82 | 365 | 427 | | C |
| ATOM | 894 | C | THR | A | 495 | −30.605 | −25.576 | 24.426 | 1.00 | 16.13 | | | C |
| ANISOU | 894 | C | THR | A | 495 | 1903 | 2188 | 2038 | −140 | 315 | 487 | | C |
| ATOM | 895 | O | THR | A | 495 | −29.382 | −25.758 | 24.397 | 1.00 | 10.85 | | | O |
| ANISOU | 895 | O | THR | A | 495 | 1293 | 1481 | 1347 | −122 | 288 | 488 | | O |
| ATOM | 896 | N | GLN | A | 496 | −31.471 | −26.478 | 23.958 | 1.00 | 12.88 | | | N |
| ANISOU | 896 | N | GLN | A | 496 | 1427 | 1694 | 1772 | −216 | 349 | 523 | | N |
| ATOM | 897 | CA | GLN | A | 496 | −31.001 | −27.658 | 23.233 | 1.00 | 17.91 | | | C |
| ANISOU | 897 | CA | GLN | A | 496 | 2068 | 2170 | 2567 | −283 | 347 | 531 | | C |
| ATOM | 898 | CB | GLN | A | 496 | −32.194 | −28.548 | 22.855 | 1.00 | 18.03 | | | C |
| ANISOU | 898 | CB | GLN | A | 496 | 1979 | 2103 | 2767 | −375 | 388 | 543 | | C |
| ATOM | 899 | CG | GLN | A | 496 | −32.955 | −29.166 | 24.041 | 1.00 | 46.56 | | | C |
| ANISOU | 899 | CG | GLN | A | 496 | 5527 | 5760 | 6403 | −362 | 477 | 689 | | C |
| ATOM | 900 | CD | GLN | A | 496 | −33.628 | −28.146 | 24.972 | 1.00 | 47.22 | | | C |
| ANISOU | 900 | CD | GLN | A | 496 | 5576 | 6038 | 6330 | −300 | 517 | 744 | | C |
| ATOM | 901 | OE1 | GLN | A | 496 | −33.580 | −28.289 | 26.200 | 1.00 | 57.96 | | | O |
| ANISOU | 901 | OE1 | GLN | A | 496 | 6932 | 7494 | 7596 | −229 | 566 | 855 | | O |
| ATOM | 902 | NE2 | GLN | A | 496 | −34.256 | −27.126 | 24.396 | 1.00 | 25.60 | | | N |
| ANISOU | 902 | NE2 | GLN | A | 496 | 2821 | 3366 | 3540 | −300 | 470 | 624 | | N |
| ATOM | 903 | C | GLN | A | 496 | −30.215 | −27.261 | 21.986 | 1.00 | 15.79 | | | C |
| ANISOU | 903 | C | GLN | A | 496 | 1869 | 1843 | 2286 | −292 | 256 | 383 | | C |
| ATOM | 904 | O | GLN | A | 496 | −29.215 | −27.906 | 21.635 | 1.00 | 10.88 | | | O |
| ANISOU | 904 | O | GLN | A | 496 | 1287 | 1130 | 1717 | −300 | 243 | 378 | | O |
| ATOM | 905 | N | GLN | A | 497 | −30.646 | −26.194 | 21.307 | 1.00 | 9.26 | | | N |
| ANISOU | 905 | N | GLN | A | 497 | 1051 | 1076 | 1390 | −280 | 198 | 276 | | N |
| ATOM | 906 | CA | GLN | A | 497 | −29.907 | −25.715 | 20.142 | 1.00 | 8.41 | | | C |
| ANISOU | 906 | CA | GLN | A | 497 | 1001 | 943 | 1251 | −274 | 125 | 170 | | C |
| ATOM | 907 | CB | GLN | A | 497 | −30.746 | −24.701 | 19.362 | 1.00 | 8.21 | | | C |
| ANISOU | 907 | CB | GLN | A | 497 | 952 | 987 | 1179 | −259 | 79 | 95 | | C |
| ATOM | 908 | CG | GLN | A | 497 | −31.994 | −25.235 | 18.723 | 1.00 | 16.40 | | | C |
| ANISOU | 908 | CG | GLN | A | 497 | 1897 | 2035 | 2300 | −312 | 71 | 46 | | C |
| ATOM | 909 | CD | GLN | A | 497 | −32.834 | −24.102 | 18.165 | 1.00 | 20.14 | | | C |
| ANISOU | 909 | CD | GLN | A | 497 | 2342 | 2607 | 2704 | −267 | 28 | 2 | | C |
| ATOM | 910 | OE1 | GLN | A | 497 | −33.682 | −23.551 | 18.859 | 1.00 | 20.15 | | | O |
| ANISOU | 910 | OE1 | GLN | A | 497 | 2304 | 2667 | 2685 | −238 | 55 | 37 | | O |
| ATOM | 911 | NE2 | GLN | A | 497 | −32.569 | −23.716 | 16.919 | 1.00 | 18.99 | | | N |
| ANISOU | 911 | NE2 | GLN | A | 497 | 2209 | 2491 | 2513 | −247 | −35 | −63 | | N |
| ATOM | 912 | C | GLN | A | 497 | −28.576 | −25.080 | 20.546 | 1.00 | 8.64 | | | C |
| ANISOU | 912 | C | GLN | A | 497 | 1110 | 988 | 1184 | −218 | 110 | 182 | | C |
| ATOM | 913 | O | GLN | A | 497 | −27.583 | −25.199 | 19.828 | 1.00 | 8.98 | | | O |
| ANISOU | 913 | O | GLN | A | 497 | 1196 | 984 | 1231 | −222 | 78 | 141 | | O |
| ATOM | 914 | N | LEU | A | 498 | −28.560 | −24.340 | 21.653 | 1.00 | 7.69 | | | N |
| ANISOU | 914 | N | LEU | A | 498 | 997 | 949 | 975 | −164 | 130 | 218 | | N |
| ATOM | 915 | CA | LEU | A | 498 | −27.313 | −23.745 | 22.114 | 1.00 | 7.27 | | | C |
| ANISOU | 915 | CA | LEU | A | 498 | 997 | 921 | 845 | −117 | 109 | 203 | | C |
| ATOM | 916 | CB | LEU | A | 498 | −27.585 | −22.856 | 23.334 | 1.00 | 10.16 | | | C |
| ANISOU | 916 | CB | LEU | A | 498 | 1345 | 1403 | 1114 | −54 | 124 | 196 | | C |
| ATOM | 917 | CG | LEU | A | 498 | −28.555 | −21.697 | 23.065 | 1.00 | 9.86 | | | C |
| ANISOU | 917 | CG | LEU | A | 498 | 1287 | 1388 | 1072 | −36 | 109 | 124 | | C |
| ATOM | 918 | CD1 | LEU | A | 498 | −28.743 | −20.825 | 24.307 | 1.00 | 11.69 | | | C |
| ANISOU | 918 | CD1 | LEU | A | 498 | 1492 | 1734 | 1215 | 36 | 123 | 80 | | C |
| ATOM | 919 | CD2 | LEU | A | 498 | −28.073 | −20.865 | 21.868 | 1.00 | 10.39 | | | C |
| ANISOU | 919 | CD2 | LEU | A | 498 | 1396 | 1373 | 1180 | −46 | 58 | 58 | | C |
| ATOM | 920 | C | LEU | A | 498 | −26.287 | −24.827 | 22.441 | 1.00 | 7.47 | | | C |
| ANISOU | 920 | C | LEU | A | 498 | 1038 | 908 | 893 | −118 | 126 | 270 | | C |
| ATOM | 921 | O | LEU | A | 498 | −25.112 | −24.701 | 22.085 | 1.00 | 9.27 | | | O |
| ANISOU | 921 | O | LEU | A | 498 | 1306 | 1107 | 1109 | −109 | 93 | 234 | | O |
| ATOM | 922 | N | LEU | A | 499 | −26.713 | −25.902 | 23.115 | 1.00 | 8.33 | | | N |
| ANISOU | 922 | N | LEU | A | 499 | 1106 | 1011 | 1048 | −124 | 185 | 382 | | N |
| ATOM | 923 | CA | LEU | A | 499 | −25.771 | −26.966 | 23.455 | 1.00 | 11.18 | | | C |
| ANISOU | 923 | CA | LEU | A | 499 | 1474 | 1323 | 1451 | −109 | 209 | 473 | | C |
| ATOM | 924 | CB | LEU | A | 499 | −26.428 | −27.959 | 24.424 | 1.00 | 10.11 | | | C |
| ANISOU | 924 | CB | LEU | A | 499 | 1277 | 1197 | 1365 | −101 | 295 | 642 | | C |
| ATOM | 925 | CG | LEU | A | 499 | −25.501 | −28.981 | 25.094 | 1.00 | 11.38 | | | C |
| ANISOU | 925 | CG | LEU | A | 499 | 1435 | 1340 | 1550 | −50 | 321 | 757 | | C |
| ATOM | 926 | CD1 | LEU | A | 499 | −24.383 | −28.267 | 25.864 | 1.00 | 11.54 | | | C |
| ANISOU | 926 | CD1 | LEU | A | 499 | 1482 | 1512 | 1391 | 39 | 287 | 762 | | C |
| ATOM | 927 | CD2 | LEU | A | 499 | −26.274 | −29.905 | 26.005 | 1.00 | 16.65 | | | C |
| ANISOU | 927 | CD2 | LEU | A | 499 | 2034 | 2022 | 2271 | −34 | 386 | 880 | | C |
| ATOM | 928 | C | LEU | A | 499 | −25.258 | −27.672 | 22.198 | 1.00 | 13.75 | | | C |
| ANISOU | 928 | C | LEU | A | 499 | 1820 | 1504 | 1899 | −159 | 183 | 409 | | C |
| ATOM | 929 | O | LEU | A | 499 | −24.083 | −28.068 | 22.126 | 1.00 | 9.24 | | | O |
| ANISOU | 929 | O | LEU | A | 499 | 1276 | 896 | 1336 | −134 | 171 | 417 | | O |
| ATOM | 930 | N | GLU | A | 500 | −26.114 | −27.813 | 21.187 | 1.00 | 8.82 | | | N |
| ANISOU | 930 | N | GLU | A | 500 | 1173 | 819 | 1361 | −222 | 170 | 328 | | N |
| ATOM | 931 | CA | GLU | A | 500 | −25.671 | −28.408 | 19.930 | 1.00 | 8.41 | | | C |
| ANISOU | 931 | CA | GLU | A | 500 | 1128 | 669 | 1399 | −259 | 138 | 228 | | C |
| ATOM | 932 | CB | GLU | A | 500 | −26.870 | −28.580 | 18.999 | 1.00 | 16.67 | | | C |
| ANISOU | 932 | CB | GLU | A | 500 | 2118 | 1693 | 2521 | −322 | 121 | 133 | | C |
| ATOM | 933 | CG | GLU | A | 500 | −26.509 | −29.186 | 17.658 | 1.00 | 24.95 | | | C |
| ANISOU | 933 | CG | GLU | A | 500 | 3155 | 2681 | 3642 | −350 | 82 | −7 | | C |
| ATOM | 934 | CD | GLU | A | 500 | −26.395 | −30.704 | 17.708 | 1.00 | 32.95 | | | C |
| ANISOU | 934 | CD | GLU | A | 500 | 4131 | 3560 | 4831 | −375 | 116 | −6 | | C |
| ATOM | 935 | OE1 | GLU | A | 500 | −26.695 | −31.286 | 18.771 | 1.00 | 37.39 | | | O |
| ANISOU | 935 | OE1 | GLU | A | 500 | 4672 | 4084 | 5449 | −369 | 172 | 125 | | O |
| ATOM | 936 | OE2 | GLU | A | 500 | −26.016 | −31.312 | 16.678 | 1.00 | 38.00 | | | O |
| ANISOU | 936 | OE2 | GLU | A | 500 | 4758 | 4170 | 5512 | −377 | 83 | −135 | | O |
| ATOM | 937 | C | GLU | A | 500 | −24.586 | −27.571 | 19.259 | 1.00 | 8.43 | | | C |
| ANISOU | 937 | C | GLU | A | 500 | 1187 | 717 | 1300 | −228 | 84 | 150 | | C |
| ATOM | 938 | O | GLU | A | 500 | −23.673 | −28.117 | 18.618 | 1.00 | 7.51 | | | O |
| ANISOU | 938 | O | GLU | A | 500 | 1085 | 546 | 1223 | −225 | 71 | 104 | | O |
| ATOM | 939 | N | MET | A | 501 | −24.688 | −26.238 | 19.348 | 1.00 | 8.39 | | | N |
| ANISOU | 939 | N | MET | A | 501 | 1204 | 802 | 1181 | −204 | 58 | 131 | | N |
| ATOM | 940 | CA | MET | A | 501 | −23.614 | −25.384 | 18.842 | 1.00 | 7.95 | | | C |
| ANISOU | 940 | CA | MET | A | 501 | 1190 | 774 | 1056 | −180 | 23 | 86 | | C |
| ATOM | 941 | CB | MET | A | 501 | −23.994 | −23.901 | 18.948 | 1.00 | 6.97 | | | C |
| ANISOU | 941 | CB | MET | A | 501 | 1074 | 711 | 862 | −161 | 6 | 69 | | C |
| ATOM | 942 | CG | MET | A | 501 | −25.217 | −23.446 | 18.156 | 1.00 | 8.96 | | | C |
| ANISOU | 942 | CG | MET | A | 501 | 1301 | 986 | 1117 | −173 | −7 | 41 | | C |
| ATOM | 943 | SD | MET | A | 501 | −25.652 | −21.778 | 18.743 | 1.00 | 13.95 | | | S |
| ANISOU | 943 | SD | MET | A | 501 | 1937 | 1659 | 1706 | −131 | −11 | 43 | | S |
| ATOM | 944 | CE | MET | A | 501 | −27.320 | −21.608 | 18.098 | 1.00 | 16.72 | | | C |
| ANISOU | 944 | CE | MET | A | 501 | 2238 | 2049 | 2067 | −132 | −19 | 36 | | C |
| ATOM | 945 | C | MET | A | 501 | −22.304 | −25.623 | 19.595 | 1.00 | 9.90 | | | C |
| ANISOU | 945 | C | MET | A | 501 | 1456 | 1021 | 1283 | −145 | 29 | 126 | | C |
| ATOM | 946 | O | MET | A | 501 | −21.224 | −25.650 | 18.985 | 1.00 | 9.28 | | | O |
| ANISOU | 946 | O | MET | A | 501 | 1393 | 928 | 1204 | −138 | 13 | 91 | | O |
| ATOM | 947 | N | CYS | A | 502 | −22.371 | −25.756 | 20.919 | 1.00 | 6.75 | | | N |
| ANISOU | 947 | N | CYS | A | 502 | 1046 | 667 | 853 | −112 | 53 | 202 | | N |
| ATOM | 948 | CA | CYS | A | 502 | −21.170 | −26.087 | 21.688 | 1.00 | 7.58 | | | C |
| ANISOU | 948 | CA | CYS | A | 502 | 1152 | 804 | 925 | −63 | 53 | 248 | | C |
| ATOM | 949 | CB | CYS | A | 502 | −21.481 | −26.143 | 23.186 | 1.00 | 9.26 | | | C |
| ANISOU | 949 | CB | CYS | A | 502 | 1335 | 1120 | 1062 | −10 | 80 | 340 | | C |
| ATOM | 950 | SG | CYS | A | 502 | −22.030 | −24.593 | 23.885 | 1.00 | 12.29 | | | S |
| ANISOU | 950 | SG | CYS | A | 502 | 1708 | 1632 | 1330 | 12 | 57 | 261 | | S |
| ATOM | 951 | C | CYS | A | 502 | −20.582 | −27.423 | 21.254 | 1.00 | 8.16 | | | C |
| ANISOU | 951 | C | CYS | A | 502 | 1222 | 780 | 1099 | −63 | 71 | 285 | | C |
| ATOM | 952 | O | CYS | A | 502 | −19.358 | −27.566 | 21.145 | 1.00 | 8.31 | | | O |
| ANISOU | 952 | O | CYS | A | 502 | 1246 | 800 | 1109 | −33 | 54 | 272 | | O |
| ATOM | 953 | N | LYS | A | 503 | −21.439 | −28.418 | 21.034 | 1.00 | 8.10 | | | N |
| ANISOU | 953 | N | LYS | A | 503 | 1193 | 679 | 1205 | −95 | 109 | 322 | | N |
| ATOM | 954 | CA | LYS | A | 503 | −20.965 | −29.738 | 20.635 | 1.00 | 10.44 | | | C |
| ANISOU | 954 | CA | LYS | A | 503 | 1476 | 849 | 1642 | −95 | 132 | 339 | | C |
| ATOM | 955 | CB | LYS | A | 503 | −22.118 | −30.734 | 20.642 | 1.00 | 9.78 | | | C |
| ANISOU | 955 | CB | LYS | A | 503 | 1348 | 657 | 1712 | −142 | 180 | 379 | | C |
| ATOM | 956 | CG | LYS | A | 503 | −21.633 | −32.142 | 20.366 | 1.00 | 14.59 | | | C |
| ANISOU | 956 | CG | LYS | A | 503 | 1930 | 1155 | 2457 | −128 | 195 | 368 | | C |
| ATOM | 957 | CD | LYS | A | 503 | −22.694 | −33.162 | 20.726 | 1.00 | 22.92 | | | C |
| ANISOU | 957 | CD | LYS | A | 503 | 2926 | 2145 | 3638 | −157 | 241 | 420 | | C |
| ATOM | 958 | CE | LYS | A | 503 | −23.684 | −33.356 | 19.601 | 1.00 | 22.18 | | | C |
| ANISOU | 958 | CE | LYS | A | 503 | 2799 | 1999 | 3631 | −234 | 220 | 268 | | C |
| ATOM | 959 | NZ | LYS | A | 503 | −23.077 | −34.046 | 18.420 | 1.00 | 23.01 | | | N |
| ANISOU | 959 | NZ | LYS | A | 503 | 2895 | 2028 | 3821 | −237 | 191 | 118 | | N |
| ATOM | 960 | C | LYS | A | 503 | −20.326 | −29.697 | 19.258 | 1.00 | 11.16 | | | C |
| ANISOU | 960 | C | LYS | A | 503 | 1583 | 906 | 1753 | −114 | 94 | 195 | | C |
| ATOM | 961 | O | LYS | A | 503 | −19.271 | −30.315 | 19.036 | 1.00 | 12.94 | | | O |
| ANISOU | 961 | O | LYS | A | 503 | 1808 | 1085 | 2025 | −80 | 95 | 186 | | O |
| ATOM | 962 | N | ASP | A | 504 | −20.961 | −28.982 | 18.323 | 1.00 | 9.03 | | | N |
| ANISOU | 962 | N | ASP | A | 504 | 1317 | 675 | 1439 | −157 | 64 | 92 | | N |
| ATOM | 963 | CA | ASP | A | 504 | −20.373 | −28.739 | 17.005 | 1.00 | 11.61 | | | C |
| ANISOU | 963 | CA | ASP | A | 504 | 1650 | 1026 | 1734 | −160 | 32 | −26 | | C |
| ATOM | 964 | CB | ASP | A | 504 | −21.176 | −27.678 | 16.234 | 1.00 | 11.11 | | | C |
| ANISOU | 964 | CB | ASP | A | 504 | 1587 | 1049 | 1584 | −185 | 3 | −81 | | C |
| ATOM | 965 | CG | ASP | A | 504 | −22.492 | −28.203 | 15.660 | 1.00 | 13.00 | | | C |
| ANISOU | 965 | CG | ASP | A | 504 | 1784 | 1268 | 1889 | −228 | −4 | −150 | | C |
| ATOM | 966 | OD1 | ASP | A | 504 | −22.651 | −29.436 | 15.494 | 1.00 | 13.90 | | | O |
| ANISOU | 966 | OD1 | ASP | A | 504 | 1862 | 1285 | 2136 | −250 | 10 | −201 | | O |
| ATOM | 967 | OD2 | ASP | A | 504 | −23.364 | −27.356 | 15.328 | 1.00 | 17.52 | | | O |
| ANISOU | 967 | OD2 | ASP | A | 504 | 2347 | 1918 | 2393 | −237 | −24 | −161 | | O |
| ATOM | 968 | C | ASP | A | 504 | −18.936 | −28.268 | 17.127 | 1.00 | 12.42 | | | C |
| ANISOU | 968 | C | ASP | A | 504 | 1774 | 1179 | 1766 | −117 | 22 | −9 | | C |
| ATOM | 969 | O | ASP | A | 504 | −18.027 | −28.834 | 16.508 | 1.00 | 7.25 | | | O |
| ANISOU | 969 | O | ASP | A | 504 | 1109 | 501 | 1143 | −95 | 23 | −61 | | O |
| ATOM | 970 | N | VAL | A | 505 | −18.724 | −27.181 | 17.875 | 1.00 | 8.43 | | | N |
| ANISOU | 970 | N | VAL | A | 505 | 1285 | 746 | 1172 | −105 | 11 | 41 | | N |
| ATOM | 971 | CA | VAL | A | 505 | −17.382 | −26.624 | 18.010 | 1.00 | 8.52 | | | C |
| ANISOU | 971 | CA | VAL | A | 505 | 1296 | 808 | 1132 | −78 | −2 | 39 | | C |
| ATOM | 972 | CB | VAL | A | 505 | −17.455 | −25.264 | 18.726 | 1.00 | 10.86 | | | C |
| ANISOU | 972 | CB | VAL | A | 505 | 1597 | 1172 | 1359 | −82 | −19 | 49 | | C |
| ATOM | 973 | CG1 | VAL | A | 505 | −16.059 | −24.741 | 19.069 | 1.00 | 13.02 | | | C |
| ANISOU | 973 | CG1 | VAL | A | 505 | 1848 | 1494 | 1607 | −62 | −36 | 32 | | C |
| ATOM | 974 | CG2 | VAL | A | 505 | −18.172 | −24.274 | 17.846 | 1.00 | 8.38 | | | C |
| ANISOU | 974 | CG2 | VAL | A | 505 | 1292 | 862 | 1029 | −117 | −23 | 15 | | C |
| ATOM | 975 | C | VAL | A | 505 | −16.458 | −27.606 | 18.728 | 1.00 | 10.94 | | | C |
| ANISOU | 975 | C | VAL | A | 505 | 1587 | 1093 | 1477 | −26 | 8 | 90 | | C |
| ATOM | 976 | O | VAL | A | 505 | −15.289 | −27.769 | 18.358 | 1.00 | 8.40 | | | O |
| ANISOU | 976 | O | VAL | A | 505 | 1249 | 782 | 1159 | −1 | 3 | 63 | | O |
| ATOM | 977 | N | CYS | A | 506 | −16.971 | −28.294 | 19.746 | 1.00 | 9.38 | | | N |
| ANISOU | 977 | N | CYS | A | 506 | 1383 | 871 | 1311 | −2 | 29 | 182 | | N |
| ATOM | 978 | CA | CYS | A | 506 | −16.123 | −29.196 | 20.520 | 1.00 | 8.08 | | | C |
| ANISOU | 978 | CA | CYS | A | 506 | 1195 | 697 | 1177 | 67 | 42 | 270 | | C |
| ATOM | 979 | CB | CYS | A | 506 | −16.893 | −29.695 | 21.750 | 1.00 | 8.80 | | | C |
| ANISOU | 979 | CB | CYS | A | 506 | 1272 | 798 | 1273 | 98 | 77 | 412 | | C |
| ATOM | 980 | SG | CYS | A | 506 | −15.842 | −30.486 | 23.001 | 1.00 | 12.49 | | | S |
| ANISOU | 980 | SG | CYS | A | 506 | 1698 | 1329 | 1717 | 216 | 90 | 571 | | S |
| ATOM | 981 | C | CYS | A | 506 | −15.622 | −30.371 | 19.675 | 1.00 | 9.93 | | | C |
| ANISOU | 981 | C | CYS | A | 506 | 1419 | 811 | 1543 | 81 | 62 | 238 | | C |
| ATOM | 982 | O | CYS | A | 506 | −14.476 | −30.812 | 19.828 | 1.00 | 9.14 | | | O |
| ANISOU | 982 | O | CYS | A | 506 | 1297 | 717 | 1458 | 143 | 60 | 260 | | O |
| ATOM | 983 | N | GLU | A | 507 | −16.462 | −30.904 | 18.791 | 1.00 | 8.82 | | | N |
| ANISOU | 983 | N | GLU | A | 507 | 1280 | 567 | 1503 | 31 | 78 | 168 | | N |
| ATOM | 984 | CA | GLU | A | 507 | −16.000 | −31.973 | 17.912 | 1.00 | 9.59 | | | C |
| ANISOU | 984 | CA | GLU | A | 507 | 1357 | 554 | 1733 | 46 | 92 | 86 | | C |
| ATOM | 985 | CB | GLU | A | 507 | −17.174 | −32.529 | 17.095 | 1.00 | 12.55 | | | C |
| ANISOU | 985 | CB | GLU | A | 507 | 1717 | 830 | 2223 | −18 | 102 | −15 | | C |
| ATOM | 986 | CG | GLU | A | 507 | −18.222 | −33.238 | 17.945 | 1.00 | 10.82 | | | C |
| ANISOU | 986 | CG | GLU | A | 507 | 1476 | 540 | 2095 | −41 | 136 | 93 | | C |
| ATOM | 987 | CD | GLU | A | 507 | −19.532 | −33.441 | 17.216 | 1.00 | 23.28 | | | C |
| ANISOU | 987 | CD | GLU | A | 507 | 3026 | 2103 | 3717 | −114 | 127 | −14 | | C |
| ATOM | 988 | OE1 | GLU | A | 507 | −20.323 | −34.301 | 17.637 | 1.00 | 22.95 | | | O |
| ANISOU | 988 | OE1 | GLU | A | 507 | 2943 | 1993 | 3782 | −135 | 158 | 37 | | O |
| ATOM | 989 | OE2 | GLU | A | 507 | −19.783 | −32.736 | 16.227 | 1.00 | 23.89 | | | O |
| ANISOU | 989 | OE2 | GLU | A | 507 | 3110 | 2247 | 3721 | −145 | 89 | −144 | | O |
| ATOM | 990 | C | GLU | A | 507 | −14.881 | −31.486 | 16.998 | 1.00 | 9.19 | | | C |
| ANISOU | 990 | C | GLU | A | 507 | 1304 | 587 | 1602 | 65 | 66 | −19 | | C |
| ATOM | 991 | O | GLU | A | 507 | −13.888 | −32.193 | 16.780 | 1.00 | 9.86 | | | O |
| ANISOU | 991 | O | GLU | A | 507 | 1364 | 634 | 1750 | 121 | 76 | −43 | | O |
| ATOM | 992 | N | ALA | A | 508 | −15.018 | −30.285 | 16.448 | 1.00 | 8.29 | | | N |
| ANISOU | 992 | N | ALA | A | 508 | 1206 | 585 | 1360 | 25 | 42 | −71 | | N |
| ATOM | 993 | CA | ALA | A | 508 | −13.952 | −29.760 | 15.597 | 1.00 | 11.33 | | | C |
| ANISOU | 993 | CA | ALA | A | 508 | 1575 | 1058 | 1672 | 40 | 34 | −139 | | C |
| ATOM | 994 | CB | ALA | A | 508 | −14.378 | −28.434 | 14.977 | 1.00 | 7.41 | | | C |
| ANISOU | 994 | CB | ALA | A | 508 | 1092 | 660 | 1064 | −8 | 22 | −158 | | C |
| ATOM | 995 | C | ALA | A | 508 | −12.664 | −29.575 | 16.387 | 1.00 | 10.06 | | | C |
| ANISOU | 995 | C | ALA | A | 508 | 1395 | 944 | 1485 | 88 | 29 | −73 | | C |
| ATOM | 996 | O | ALA | A | 508 | −11.568 | −29.869 | 15.890 | 1.00 | 8.61 | | | O |
| ANISOU | 996 | O | ALA | A | 508 | 1176 | 785 | 1311 | 128 | 37 | −116 | | O |
| ATOM | 997 | N | MET | A | 509 | −12.774 | −29.057 | 17.617 | 1.00 | 9.27 | | | N |
| ANISOU | 997 | N | MET | A | 509 | 1302 | 880 | 1342 | 91 | 13 | 17 | | N |
| ATOM | 998 | CA | MET | A | 509 | −11.583 | −28.834 | 18.434 | 1.00 | 10.06 | | | C |
| ANISOU | 998 | CA | MET | A | 509 | 1363 | 1056 | 1401 | 140 | −6 | 59 | | C |
| ATOM | 999 | CB | MET | A | 509 | −11.925 | −27.948 | 19.638 | 1.00 | 8.88 | | | C |
| ANISOU | 999 | CB | MET | A | 509 | 1216 | 989 | 1171 | 132 | −34 | 106 | | C |
| ATOM | 1000 | CG | MET | A | 509 | −12.297 | −26.482 | 19.262 | 1.00 | 8.08 | | | C |
| ANISOU | 1000 | CG | MET | A | 509 | 1126 | 925 | 1019 | 57 | −48 | 41 | | C |
| ATOM | 1001 | SD | MET | A | 509 | −11.052 | −25.613 | 18.239 | 1.00 | 13.74 | | | S |
| ANISOU | 1001 | SD | MET | A | 509 | 1798 | 1682 | 1740 | 23 | −45 | −29 | | S |
| ATOM | 1002 | CE | MET | A | 509 | −9.631 | −25.669 | 19.326 | 1.00 | 14.54 | | | C |
| ANISOU | 1002 | CE | MET | A | 509 | 1822 | 1879 | 1823 | 78 | −82 | −30 | | C |
| ATOM | 1003 | C | MET | A | 509 | −10.955 | −30.143 | 18.891 | 1.00 | 9.17 | | | C |
| ANISOU | 1003 | C | MET | A | 509 | 1223 | 893 | 1367 | 227 | 7 | 121 | | C |
| ATOM | 1004 | O | MET | A | 509 | −9.729 | −30.222 | 19.036 | 1.00 | 12.50 | | | O |
| ANISOU | 1004 | O | MET | A | 509 | 1597 | 1375 | 1777 | 281 | −5 | 121 | | O |
| ATOM | 1005 | N | GLU | A | 510 | −11.759 | −31.173 | 19.153 | 1.00 | 11.33 | | | N |
| ANISOU | 1005 | N | GLU | A | 510 | 1515 | 1051 | 1741 | 244 | 37 | 184 | | N |
| ATOM | 1006 | CA | GLU | A | 510 | −11.153 | −32.460 | 19.469 | 1.00 | 13.81 | | | C |
| ANISOU | 1006 | CA | GLU | A | 510 | 1796 | 1279 | 2171 | 333 | 62 | 257 | | C |
| ATOM | 1007 | CB | GLU | A | 510 | −12.205 | −33.472 | 19.919 | 1.00 | 17.68 | | | C |
| ANISOU | 1007 | CB | GLU | A | 510 | 2298 | 1620 | 2799 | 337 | 107 | 360 | | C |
| ATOM | 1008 | CG | GLU | A | 510 | −11.561 | −34.745 | 20.416 | 1.00 | 19.82 | | | C |
| ANISOU | 1008 | CG | GLU | A | 510 | 2529 | 1790 | 3213 | 443 | 142 | 481 | | C |
| ATOM | 1009 | CD | GLU | A | 510 | −12.348 | −35.991 | 20.109 | 1.00 | 39.08 | | | C |
| ANISOU | 1009 | CD | GLU | A | 510 | 4965 | 4062 | 5823 | 399 | 184 | 461 | | C |
| ATOM | 1010 | OE1 | GLU | A | 510 | −11.880 | −37.085 | 20.503 | 1.00 | 42.67 | | | O |
| ANISOU | 1010 | OE1 | GLU | A | 510 | 5385 | 4453 | 6374 | 458 | 210 | 539 | | O |
| ATOM | 1011 | OE2 | GLU | A | 510 | −13.422 | −35.884 | 19.478 | 1.00 | 49.43 | | | O |
| ANISOU | 1011 | OE2 | GLU | A | 510 | 6294 | 5318 | 7168 | 305 | 188 | 362 | | O |
| ATOM | 1012 | C | GLU | A | 510 | −10.399 | −33.001 | 18.267 | 1.00 | 11.33 | | | C |
| ANISOU | 1012 | C | GLU | A | 510 | 1459 | 906 | 1937 | 352 | 73 | 131 | | C |
| ATOM | 1013 | O | GLU | A | 510 | −9.365 | −33.658 | 18.416 | 1.00 | 15.91 | | | O |
| ANISOU | 1013 | O | GLU | A | 510 | 1997 | 1474 | 2573 | 439 | 79 | 158 | | O |
| ATOM | 1014 | N | TYR | A | 511 | −10.915 | −32.752 | 17.068 | 1.00 | 11.98 | | | N |
| ANISOU | 1014 | N | TYR | A | 511 | 1561 | 972 | 2019 | 283 | 76 | −9 | | N |
| ATOM | 1015 | CA | TYR | A | 511 | −10.212 | −33.178 | 15.862 | 1.00 | 11.37 | | | C |
| ANISOU | 1015 | CA | TYR | A | 511 | 1452 | 888 | 1981 | 309 | 88 | −150 | | C |
| ATOM | 1016 | CB | TYR | A | 511 | −11.092 | −32.949 | 14.635 | 1.00 | 11.10 | | | C |
| ANISOU | 1016 | CB | TYR | A | 511 | 1433 | 864 | 1920 | 240 | 87 | −294 | | C |
| ATOM | 1017 | CG | TYR | A | 511 | −10.344 | −33.168 | 13.336 | 1.00 | 11.72 | | | C |
| ANISOU | 1017 | CG | TYR | A | 511 | 1467 | 1007 | 1977 | 275 | 99 | −450 | | C |
| ATOM | 1018 | CD1 | TYR | A | 511 | −10.102 | −34.445 | 12.870 | 1.00 | 13.08 | | | C |
| ANISOU | 1018 | CD1 | TYR | A | 511 | 1603 | 1067 | 2302 | 333 | 119 | −564 | | C |
| ATOM | 1019 | CE1 | TYR | A | 511 | −9.404 | −34.662 | 11.688 | 1.00 | 13.86 | | | C |
| ANISOU | 1019 | CE1 | TYR | A | 511 | 1650 | 1252 | 2364 | 378 | 132 | −727 | | C |
| ATOM | 1020 | CZ | TYR | A | 511 | −8.952 | −33.589 | 10.959 | 1.00 | 19.85 | | | C |
| ANISOU | 1020 | CZ | TYR | A | 511 | 2395 | 2219 | 2928 | 363 | 134 | −742 | | C |
| ATOM | 1021 | OH | TYR | A | 511 | −8.267 | −33.816 | 9.789 | 1.00 | 16.02 | | | O |
| ANISOU | 1021 | OH | TYR | A | 511 | 1848 | 1853 | 2386 | 418 | 157 | −890 | | O |
| ATOM | 1022 | CE2 | TYR | A | 511 | −9.172 | −32.303 | 11.397 | 1.00 | 14.74 | | | C |
| ANISOU | 1022 | CE2 | TYR | A | 511 | 1785 | 1661 | 2156 | 298 | 120 | −606 | | C |
| ATOM | 1023 | CD2 | TYR | A | 511 | −9.878 | −32.097 | 12.588 | 1.00 | 12.68 | | | C |
| ANISOU | 1023 | CD2 | TYR | A | 511 | 1576 | 1304 | 1938 | 254 | 98 | −478 | | C |
| ATOM | 1024 | C | TYR | A | 511 | −8.888 | −32.437 | 15.714 | 1.00 | 12.91 | | | C |
| ANISOU | 1024 | C | TYR | A | 511 | 1609 | 1233 | 2065 | 336 | 75 | −160 | | C |
| ATOM | 1025 | O | TYR | A | 511 | −7.842 | −33.050 | 15.457 | 1.00 | 13.69 | | | O |
| ANISOU | 1025 | O | TYR | A | 511 | 1657 | 1330 | 2213 | 411 | 88 | −195 | | O |
| ATOM | 1026 | N | LEU | A | 512 | −8.918 | −31.111 | 15.868 | 1.00 | 10.14 | | | N |
| ANISOU | 1026 | N | LEU | A | 512 | 1269 | 1001 | 1582 | 276 | 54 | −135 | | N |
| ATOM | 1027 | CA | LEU | A | 512 | −7.690 | −30.327 | 15.787 | 1.00 | 11.21 | | | C |
| ANISOU | 1027 | CA | LEU | A | 512 | 1351 | 1264 | 1644 | 283 | 46 | −141 | | C |
| ATOM | 1028 | CB | LEU | A | 512 | −8.001 | −28.843 | 15.919 | 1.00 | 10.53 | | | C |
| ANISOU | 1028 | CB | LEU | A | 512 | 1278 | 1257 | 1466 | 199 | 30 | −122 | | C |
| ATOM | 1029 | CG | LEU | A | 512 | −8.707 | −28.203 | 14.717 | 1.00 | 13.62 | | | C |
| ANISOU | 1029 | CG | LEU | A | 512 | 1697 | 1668 | 1812 | 136 | 54 | −171 | | C |
| ATOM | 1030 | CD1 | LEU | A | 512 | −8.896 | −26.707 | 14.968 | 1.00 | 12.93 | | | C |
| ANISOU | 1030 | CD1 | LEU | A | 512 | 1611 | 1627 | 1674 | 65 | 44 | −130 | | C |
| ATOM | 1031 | CD2 | LEU | A | 512 | −7.897 | −28.442 | 13.440 | 1.00 | 9.43 | | | C |
| ANISOU | 1031 | CD2 | LEU | A | 512 | 1115 | 1206 | 1262 | 165 | 92 | −242 | | C |
| ATOM | 1032 | C | LEU | A | 512 | −6.701 | −30.753 | 16.860 | 1.00 | 14.57 | | | C |
| ANISOU | 1032 | C | LEU | A | 512 | 1727 | 1715 | 2094 | 364 | 26 | −67 | | C |
| ATOM | 1033 | O | LEU | A | 512 | −5.497 | −30.821 | 16.610 | 1.00 | 13.60 | | | O |
| ANISOU | 1033 | O | LEU | A | 512 | 1536 | 1661 | 1971 | 410 | 30 | −97 | | O |
| ATOM | 1034 | N | GLU | A | 513 | −7.195 | −31.039 | 18.062 | 1.00 | 12.05 | | | N |
| ANISOU | 1034 | N | GLU | A | 513 | 1429 | 1366 | 1784 | 392 | 6 | 37 | | N |
| ATOM | 1035 | CA | GLU | A | 513 | −6.312 | −31.496 | 19.123 | 1.00 | 15.07 | | | C |
| ANISOU | 1035 | CA | GLU | A | 513 | 1754 | 1804 | 2166 | 492 | −16 | 128 | | C |
| ATOM | 1036 | CB | GLU | A | 513 | −7.083 | −31.545 | 20.444 | 1.00 | 13.12 | | | C |
| ANISOU | 1036 | CB | GLU | A | 513 | 1533 | 1574 | 1879 | 514 | −33 | 257 | | C |
| ATOM | 1037 | CG | GLU | A | 513 | −6.196 | −31.817 | 21.676 | 1.00 | 18.34 | | | C |
| ANISOU | 1037 | CG | GLU | A | 513 | 2121 | 2364 | 2485 | 630 | −68 | 366 | | C |
| ATOM | 1038 | CD | GLU | A | 513 | −6.866 | −31.391 | 22.986 | 1.00 | 35.32 | | | C |
| ANISOU | 1038 | CD | GLU | A | 513 | 4276 | 4626 | 4517 | 644 | −95 | 461 | | C |
| ATOM | 1039 | OE1 | GLU | A | 513 | −7.801 | −32.086 | 23.459 | 1.00 | 18.97 | | | O |
| ANISOU | 1039 | OE1 | GLU | A | 513 | 2245 | 2476 | 2487 | 671 | −56 | 591 | | O |
| ATOM | 1040 | OE2 | GLU | A | 513 | −6.464 | −30.336 | 23.527 | 1.00 | 44.51 | | | O |
| ANISOU | 1040 | OE2 | GLU | A | 513 | 5395 | 5962 | 5555 | 624 | −152 | 393 | | O |
| ATOM | 1041 | C | GLU | A | 513 | −5.697 | −32.848 | 18.773 | 1.00 | 15.96 | | | C |
| ANISOU | 1041 | C | GLU | A | 513 | 1835 | 1819 | 2408 | 593 | 15 | 137 | | C |
| ATOM | 1042 | O | GLU | A | 513 | −4.509 | −33.084 | 19.031 | 1.00 | 18.05 | | | O |
| ANISOU | 1042 | O | GLU | A | 513 | 2026 | 2159 | 2672 | 677 | 2 | 156 | | O |
| ATOM | 1043 | N | SER | A | 514 | −6.474 | −33.736 | 18.139 | 1.00 | 15.41 | | | N |
| ANISOU | 1043 | N | SER | A | 514 | 1809 | 1580 | 2466 | 586 | 57 | 105 | | N |
| ATOM | 1044 | CA | SER | A | 514 | −5.926 | −35.029 | 17.734 | 1.00 | 15.69 | | | C |
| ANISOU | 1044 | CA | SER | A | 514 | 1808 | 1491 | 2664 | 681 | 91 | 81 | | C |
| ATOM | 1045 | CB | SER | A | 514 | −7.031 | −35.943 | 17.203 | 1.00 | 18.24 | | | C |
| ANISOU | 1045 | CB | SER | A | 514 | 2172 | 1604 | 3154 | 652 | 130 | 29 | | C |
| ATOM | 1046 | OG | SER | A | 514 | −7.377 | −35.560 | 15.880 | 1.00 | 15.87 | | | O |
| ANISOU | 1046 | OG | SER | A | 514 | 1887 | 1330 | 2813 | 574 | 133 | −162 | | O |
| ATOM | 1047 | C | SER | A | 514 | −4.846 | −34.884 | 16.669 | 1.00 | 16.84 | | | C |
| ANISOU | 1047 | C | SER | A | 514 | 1899 | 1718 | 2784 | 701 | 98 | −64 | | C |
| ATOM | 1048 | O | SER | A | 514 | −4.072 | −35.820 | 16.454 | 1.00 | 19.30 | | | O |
| ANISOU | 1048 | O | SER | A | 514 | 2158 | 1969 | 3208 | 803 | 119 | −89 | | O |
| ATOM | 1049 | N | LYS | A | 515 | −4.789 | −33.746 | 15.982 | 1.00 | 14.13 | | | N |
| ANISOU | 1049 | N | LYS | A | 515 | 1558 | 1507 | 2304 | 613 | 88 | −150 | | N |
| ATOM | 1050 | CA | LYS | A | 515 | −3.731 | −33.462 | 15.022 | 1.00 | 16.06 | | | C |
| ANISOU | 1050 | CA | LYS | A | 515 | 1735 | 1865 | 2501 | 629 | 107 | −256 | | C |
| ATOM | 1051 | CB | LYS | A | 515 | −4.319 | −32.834 | 13.748 | 1.00 | 14.98 | | | C |
| ANISOU | 1051 | CB | LYS | A | 515 | 1626 | 1782 | 2285 | 542 | 131 | −369 | | C |
| ATOM | 1052 | CG | LYS | A | 515 | −5.438 | −33.620 | 13.100 | 1.00 | 15.05 | | | C |
| ANISOU | 1052 | CG | LYS | A | 515 | 1684 | 1663 | 2371 | 532 | 145 | −466 | | C |
| ATOM | 1053 | CD | LYS | A | 515 | −4.979 | −35.023 | 12.725 | 1.00 | 21.87 | | | C |
| ANISOU | 1053 | CD | LYS | A | 515 | 2504 | 2416 | 3389 | 637 | 171 | −566 | | C |
| ATOM | 1054 | CE | LYS | A | 515 | −5.967 | −35.689 | 11.778 | 1.00 | 26.93 | | | C |
| ANISOU | 1054 | CE | LYS | A | 515 | 3164 | 2965 | 4104 | 616 | 183 | −735 | | C |
| ATOM | 1055 | NZ | LYS | A | 515 | −5.495 | −37.038 | 11.365 | 1.00 | 30.28 | | | N |
| ANISOU | 1055 | NZ | LYS | A | 515 | 3556 | 3295 | 4653 | 681 | 200 | −817 | | N |
| ATOM | 1056 | C | LYS | A | 515 | −2.668 | −32.536 | 15.595 | 1.00 | 13.88 | | | C |
| ANISOU | 1056 | C | LYS | A | 515 | 1391 | 1752 | 2131 | 625 | 79 | −200 | | C |
| ATOM | 1057 | O | LYS | A | 515 | −1.827 | −32.041 | 14.840 | 1.00 | 20.81 | | | O |
| ANISOU | 1057 | O | LYS | A | 515 | 2204 | 2744 | 2961 | 612 | 101 | −266 | | O |
| ATOM | 1058 | N | GLN | A | 516 | −2.702 | −32.267 | 16.898 | 1.00 | 16.16 | | | N |
| ANISOU | 1058 | N | GLN | A | 516 | 1679 | 2070 | 2390 | 634 | 33 | −89 | | N |
| ATOM | 1059 | CA | GLN | A | 516 | −1.751 | −31.349 | 17.545 | 1.00 | 22.52 | | | C |
| ANISOU | 1059 | CA | GLN | A | 516 | 2403 | 3039 | 3114 | 623 | −9 | −69 | | C |
| ATOM | 1060 | CB | GLN | A | 516 | −0.344 | −31.954 | 17.591 | 1.00 | 24.32 | | | C |
| ANISOU | 1060 | CB | GLN | A | 516 | 2521 | 3340 | 3380 | 736 | −10 | −74 | | C |
| ATOM | 1061 | CG | GLN | A | 516 | −0.212 | −33.149 | 18.517 | 1.00 | 37.66 | | | C |
| ANISOU | 1061 | CG | GLN | A | 516 | 4199 | 4973 | 5136 | 879 | −28 | 39 | | C |
| ATOM | 1062 | CD | GLN | A | 516 | −0.806 | −34.412 | 17.925 | 1.00 | 54.20 | | | C |
| ANISOU | 1062 | CD | GLN | A | 516 | 6355 | 6863 | 7375 | 934 | 27 | 33 | | C |
| ATOM | 1063 | OE1 | GLN | A | 516 | −0.598 | −34.719 | 16.749 | 1.00 | 61.55 | | | O |
| ANISOU | 1063 | OE1 | GLN | A | 516 | 7276 | 7748 | 8360 | 932 | 71 | −91 | | O |
| ATOM | 1064 | NE2 | GLN | A | 516 | −1.558 | −35.150 | 18.735 | 1.00 | 58.20 | | | N |
| ANISOU | 1064 | NE2 | GLN | A | 516 | 6912 | 7250 | 7952 | 984 | 27 | 162 | | N |
| ATOM | 1065 | C | GLN | A | 516 | −1.717 | −29.976 | 16.867 | 1.00 | 22.23 | | | C |
| ANISOU | 1065 | C | GLN | A | 516 | 2354 | 3081 | 3012 | 497 | 3 | −138 | | C |
| ATOM | 1066 | O | GLN | A | 516 | −0.661 | −29.359 | 16.720 | 1.00 | 20.60 | | | O |
| ANISOU | 1066 | O | GLN | A | 516 | 2050 | 2988 | 2790 | 479 | 2 | −171 | | O |
| ATOM | 1067 | N | PHE | A | 517 | −2.889 | −29.470 | 16.490 | 1.00 | 14.21 | | | N |
| ANISOU | 1067 | N | PHE | A | 517 | 1426 | 2001 | 1972 | 409 | 16 | −145 | | N |
| ATOM | 1068 | CA | PHE | A | 517 | −3.028 | −28.180 | 15.818 | 1.00 | 13.84 | | | C |
| ANISOU | 1068 | CA | PHE | A | 517 | 1375 | 2001 | 1882 | 299 | 38 | −175 | | C |
| ATOM | 1069 | CB | PHE | A | 517 | −3.790 | −28.353 | 14.498 | 1.00 | 13.42 | | | C |
| ANISOU | 1069 | CB | PHE | A | 517 | 1380 | 1905 | 1814 | 274 | 91 | −213 | | C |
| ATOM | 1070 | CG | PHE | A | 517 | −3.737 | −27.158 | 13.581 | 1.00 | 18.41 | | | C |
| ANISOU | 1070 | CG | PHE | A | 517 | 1986 | 2607 | 2400 | 191 | 134 | −210 | | C |
| ATOM | 1071 | CD1 | PHE | A | 517 | −4.595 | −26.082 | 13.755 | 1.00 | 17.77 | | | C |
| ANISOU | 1071 | CD1 | PHE | A | 517 | 1952 | 2495 | 2304 | 106 | 125 | −168 | | C |
| ATOM | 1072 | CE1 | PHE | A | 517 | −4.549 | −24.988 | 12.884 | 1.00 | 15.29 | | | C |
| ANISOU | 1072 | CE1 | PHE | A | 517 | 1609 | 2229 | 1972 | 40 | 176 | −130 | | C |
| ATOM | 1073 | CZ | PHE | A | 517 | −3.657 | −24.980 | 11.837 | 1.00 | 12.30 | | | C |
| ANISOU | 1073 | CZ | PHE | A | 517 | 1151 | 1952 | 1570 | 58 | 242 | −128 | | C |
| ATOM | 1074 | CE2 | PHE | A | 517 | −2.797 | −26.053 | 11.649 | 1.00 | 19.21 | | | C |
| ANISOU | 1074 | CE2 | PHE | A | 517 | 1978 | 2879 | 2443 | 143 | 249 | −190 | | C |
| ATOM | 1075 | CD2 | PHE | A | 517 | −2.844 | −27.133 | 12.512 | 1.00 | 24.07 | | | C |
| ANISOU | 1075 | CD2 | PHE | A | 517 | 2626 | 3423 | 3096 | 209 | 193 | −235 | | C |
| ATOM | 1076 | C | PHE | A | 517 | −3.772 | −27.244 | 16.762 | 1.00 | 19.01 | | | C |
| ANISOU | 1076 | C | PHE | A | 517 | 2068 | 2647 | 2509 | 230 | −6 | −144 | | C |
| ATOM | 1077 | O | PHE | A | 517 | −4.946 | −27.484 | 17.076 | 1.00 | 19.33 | | | O |
| ANISOU | 1077 | O | PHE | A | 517 | 2195 | 2610 | 2539 | 224 | −17 | −115 | | O |
| ATOM | 1078 | N | LEU | A | 518 | −3.108 | −26.184 | 17.221 | 1.00 | 11.03 | | | N |
| ANISOU | 1078 | N | LEU | A | 518 | 980 | 1712 | 1499 | 177 | −31 | −165 | | N |
| ATOM | 1079 | CA | LEU | A | 518 | −3.799 | −25.211 | 18.062 | 1.00 | 11.24 | | | C |
| ANISOU | 1079 | CA | LEU | A | 518 | 1031 | 1730 | 1510 | 114 | −72 | −173 | | C |
| ATOM | 1080 | CB | LEU | A | 518 | −2.832 | −24.412 | 18.934 | 1.00 | 13.95 | | | C |
| ANISOU | 1080 | CB | LEU | A | 518 | 1258 | 2174 | 1870 | 91 | −124 | −235 | | C |
| ATOM | 1081 | CG | LEU | A | 518 | −1.922 | −25.138 | 19.910 | 1.00 | 16.92 | | | C |
| ANISOU | 1081 | CG | LEU | A | 518 | 1552 | 2669 | 2206 | 194 | −182 | −241 | | C |
| ATOM | 1082 | CD1 | LEU | A | 518 | −1.227 | −24.090 | 20.770 | 1.00 | 14.26 | | | C |
| ANISOU | 1082 | CD1 | LEU | A | 518 | 1095 | 2444 | 1881 | 148 | −247 | −345 | | C |
| ATOM | 1083 | CD2 | LEU | A | 518 | −2.718 | −26.096 | 20.764 | 1.00 | 18.45 | | | C |
| ANISOU | 1083 | CD2 | LEU | A | 518 | 1823 | 2859 | 2328 | 290 | −210 | −163 | | C |
| ATOM | 1084 | C | LEU | A | 518 | −4.570 | −24.252 | 17.177 | 1.00 | 14.57 | | | C |
| ANISOU | 1084 | C | LEU | A | 518 | 1501 | 2083 | 1953 | 19 | −26 | −168 | | C |
| ATOM | 1085 | O | LEU | A | 518 | −4.115 | −23.896 | 16.092 | 1.00 | 15.81 | | | O |
| ANISOU | 1085 | O | LEU | A | 518 | 1621 | 2249 | 2136 | −17 | 31 | −160 | | O |
| ATOM | 1086 | N | HIS | A | 519 | −5.752 | −23.846 | 17.639 | 1.00 | 12.10 | | | N |
| ANISOU | 1086 | N | HIS | A | 519 | 1262 | 1715 | 1622 | −10 | −46 | −159 | | N |
| ATOM | 1087 | CA | HIS | A | 519 | −6.466 | −22.783 | 16.955 | 1.00 | 11.18 | | | C |
| ANISOU | 1087 | CA | HIS | A | 519 | 1177 | 1536 | 1535 | −90 | −10 | −145 | | C |
| ATOM | 1088 | CB | HIS | A | 519 | −7.914 | −22.743 | 17.422 | 1.00 | 12.93 | | | C |
| ANISOU | 1088 | CB | HIS | A | 519 | 1490 | 1701 | 1721 | −92 | −31 | −131 | | C |
| ATOM | 1089 | CG | HIS | A | 519 | −8.764 | −21.808 | 16.628 | 1.00 | 15.77 | | | C |
| ANISOU | 1089 | CG | HIS | A | 519 | 1886 | 1998 | 2106 | −150 | 6 | −99 | | C |
| ATOM | 1090 | ND1 | HIS | A | 519 | −8.542 | −20.448 | 16.602 | 1.00 | 12.25 | | | N |
| ANISOU | 1090 | ND1 | HIS | A | 519 | 1392 | 1516 | 1746 | −217 | 21 | −106 | | N |
| ATOM | 1091 | CE1 | HIS | A | 519 | −9.436 | −19.876 | 15.812 | 1.00 | 15.54 | | | C |
| ANISOU | 1091 | CE1 | HIS | A | 519 | 1852 | 1880 | 2173 | −240 | 60 | −43 | | C |
| ATOM | 1092 | NE2 | HIS | A | 519 | −10.225 | −20.817 | 15.325 | 1.00 | 13.35 | | | N |
| ANISOU | 1092 | NE2 | HIS | A | 519 | 1644 | 1624 | 1805 | −195 | 62 | −17 | | N |
| ATOM | 1093 | CD2 | HIS | A | 519 | −9.820 | −22.036 | 15.814 | 1.00 | 9.90 | | | C |
| ANISOU | 1093 | CD2 | HIS | A | 519 | 1212 | 1226 | 1325 | −145 | 32 | −56 | | C |
| ATOM | 1094 | C | HIS | A | 519 | −5.786 | −21.442 | 17.191 | 1.00 | 16.65 | | | C |
| ANISOU | 1094 | C | HIS | A | 519 | 1780 | 2231 | 2317 | −167 | −11 | −185 | | C |
| ATOM | 1095 | O | HIS | A | 519 | −5.521 | −20.693 | 16.241 | 1.00 | 14.75 | | | O |
| ANISOU | 1095 | O | HIS | A | 519 | 1504 | 1961 | 2141 | −224 | 50 | −144 | | O |
| ATOM | 1096 | N | ARG | A | 520 | −5.517 | −21.120 | 18.460 | 1.00 | 10.11 | | | N |
| ANISOU | 1096 | N | ARG | A | 520 | 902 | 1441 | 1498 | −166 | −78 | −265 | | N |
| ATOM | 1097 | CA | ARG | A | 520 | −4.821 | −19.951 | 18.989 | 1.00 | 11.90 | | | C |
| ANISOU | 1097 | CA | ARG | A | 520 | 1020 | 1674 | 1830 | −236 | −103 | −360 | | C |
| ATOM | 1098 | CB | ARG | A | 520 | −3.510 | −19.633 | 18.235 | 1.00 | 12.08 | | | C |
| ANISOU | 1098 | CB | ARG | A | 520 | 927 | 1712 | 1950 | −284 | −55 | −353 | | C |
| ATOM | 1099 | CG | ARG | A | 520 | −2.318 | −20.498 | 18.674 | 1.00 | 12.61 | | | C |
| ANISOU | 1099 | CG | ARG | A | 520 | 908 | 1914 | 1969 | −218 | −96 | −394 | | C |
| ATOM | 1100 | CD | ARG | A | 520 | −1.086 | −20.350 | 17.740 | 1.00 | 13.48 | | | C |
| ANISOU | 1100 | CD | ARG | A | 520 | 906 | 2052 | 2164 | −256 | −31 | −367 | | C |
| ATOM | 1101 | NE | ARG | A | 520 | 0.140 | −20.890 | 18.337 | 1.00 | 14.39 | | | N |
| ANISOU | 1101 | NE | ARG | A | 520 | 902 | 2301 | 2263 | −203 | −83 | −435 | | N |
| ATOM | 1102 | CZ | ARG | A | 520 | 0.698 | −22.053 | 18.002 | 1.00 | 14.39 | | | C |
| ANISOU | 1102 | CZ | ARG | A | 520 | 899 | 2379 | 2190 | −107 | −70 | −390 | | C |
| ATOM | 1103 | NH1 | ARG | A | 520 | 0.135 | −22.810 | 17.073 | 1.00 | 13.58 | | | N |
| ANISOU | 1103 | NH1 | ARG | A | 520 | 902 | 2229 | 2027 | −60 | −10 | −303 | | N |
| ATOM | 1104 | NH2 | ARG | A | 520 | 1.816 | −22.465 | 18.597 | 1.00 | 15.39 | | | N |
| ANISOU | 1104 | NH2 | ARG | A | 520 | 904 | 2634 | 2309 | −50 | −121 | −446 | | N |
| ATOM | 1105 | C | ARG | A | 520 | −5.699 | −18.693 | 19.037 | 1.00 | 11.10 | | | C |
| ANISOU | 1105 | C | ARG | A | 520 | 942 | 1458 | 1819 | −309 | −91 | −386 | | C |
| ATOM | 1106 | O | ARG | A | 520 | −5.265 | −17.692 | 19.610 | 1.00 | 14.04 | | | O |
| ANISOU | 1106 | O | ARG | A | 520 | 1221 | 1808 | 2306 | −369 | −118 | −496 | | O |
| ATOM | 1107 | N | ASP | A | 521 | −6.916 | −18.705 | 18.485 | 1.00 | 10.22 | | | N |
| ANISOU | 1107 | N | ASP | A | 521 | 939 | 1270 | 1673 | −303 | −55 | −302 | | N |
| ATOM | 1108 | CA | ASP | A | 521 | −7.813 | −17.561 | 18.646 | 1.00 | 11.33 | | | C |
| ANISOU | 1108 | CA | ASP | A | 521 | 1101 | 1305 | 1900 | −352 | −49 | −326 | | C |
| ATOM | 1109 | CB | ASP | A | 521 | −7.474 | −16.466 | 17.622 | 1.00 | 16.00 | | | C |
| ANISOU | 1109 | CB | ASP | A | 521 | 1638 | 1783 | 2657 | −431 | 29 | −257 | | C |
| ATOM | 1110 | CG | ASP | A | 521 | −8.260 | −15.174 | 17.866 | 1.00 | 24.49 | | | C |
| ANISOU | 1110 | CG | ASP | A | 521 | 2713 | 2717 | 3874 | −478 | 37 | −288 | | C |
| ATOM | 1111 | OD1 | ASP | A | 521 | −8.788 | −14.997 | 18.991 | 1.00 | 21.75 | | | O |
| ANISOU | 1111 | OD1 | ASP | A | 521 | 2383 | 2386 | 3496 | −451 | −29 | −411 | | O |
| ATOM | 1112 | OD2 | ASP | A | 521 | −8.356 | −14.333 | 16.941 | 1.00 | 20.59 | | | O |
| ANISOU | 1112 | OD2 | ASP | A | 521 | 2219 | 2132 | 3473 | −501 | 110 | −168 | | O |
| ATOM | 1113 | C | ASP | A | 521 | −9.284 | −17.952 | 18.527 | 1.00 | 14.01 | | | C |
| ANISOU | 1113 | C | ASP | A | 521 | 1561 | 1620 | 2143 | −308 | −46 | −263 | | C |
| ATOM | 1114 | O | ASP | A | 521 | −10.050 | −17.294 | 17.815 | 1.00 | 11.74 | | | O |
| ANISOU | 1114 | O | ASP | A | 521 | 1310 | 1244 | 1908 | −333 | −1 | −195 | | O |
| ATOM | 1115 | N | LEU | A | 522 | −9.697 | −18.998 | 19.245 | 1.00 | 10.27 | | | N |
| ANISOU | 1115 | N | LEU | A | 522 | 1136 | 1227 | 1538 | −240 | −89 | −275 | | N |
| ATOM | 1116 | CA | LEU | A | 522 | −11.073 | −19.477 | 19.192 | 1.00 | 9.95 | | | C |
| ANISOU | 1116 | CA | LEU | A | 522 | 1192 | 1168 | 1421 | −206 | −84 | −219 | | C |
| ATOM | 1117 | CB | LEU | A | 522 | −11.178 | −20.841 | 19.877 | 1.00 | 10.25 | | | C |
| ANISOU | 1117 | CB | LEU | A | 522 | 1259 | 1288 | 1348 | −134 | −112 | −199 | | C |
| ATOM | 1118 | CG | LEU | A | 522 | −12.565 | −21.494 | 19.910 | 1.00 | 11.06 | | | C |
| ANISOU | 1118 | CG | LEU | A | 522 | 1442 | 1368 | 1392 | −106 | −102 | −140 | | C |
| ATOM | 1119 | CD1 | LEU | A | 522 | −13.018 | −21.830 | 18.476 | 1.00 | 10.93 | | | C |
| ANISOU | 1119 | CD1 | LEU | A | 522 | 1470 | 1290 | 1393 | −125 | −57 | −82 | | C |
| ATOM | 1120 | CD2 | LEU | A | 522 | −12.568 | −22.754 | 20.793 | 1.00 | 9.99 | | | C |
| ANISOU | 1120 | CD2 | LEU | A | 522 | 1313 | 1298 | 1184 | −36 | −119 | −99 | | C |
| ATOM | 1121 | C | LEU | A | 522 | −12.027 | −18.479 | 19.850 | 1.00 | 13.67 | | | C |
| ANISOU | 1121 | C | LEU | A | 522 | 1673 | 1595 | 1928 | −221 | −101 | −277 | | C |
| ATOM | 1122 | O | LEU | A | 522 | −11.796 | −18.028 | 20.977 | 1.00 | 12.07 | | | O |
| ANISOU | 1122 | O | LEU | A | 522 | 1419 | 1440 | 1729 | −215 | −147 | −390 | | O |
| ATOM | 1123 | N | ALA | A | 523 | −13.097 | −18.124 | 19.140 | 1.00 | 10.68 | | | N |
| ANISOU | 1123 | N | ALA | A | 523 | 1349 | 1141 | 1568 | −232 | −66 | −212 | | N |
| ATOM | 1124 | CA | ALA | A | 523 | −14.085 | −17.196 | 19.674 | 1.00 | 7.82 | | | C |
| ANISOU | 1124 | CA | ALA | A | 523 | 994 | 727 | 1248 | −234 | −75 | −261 | | C |
| ATOM | 1125 | CB | ALA | A | 523 | −13.539 | −15.767 | 19.742 | 1.00 | 8.89 | | | C |
| ANISOU | 1125 | CB | ALA | A | 523 | 1058 | 765 | 1557 | −287 | −67 | −335 | | C |
| ATOM | 1126 | C | ALA | A | 523 | −15.316 | −17.273 | 18.790 | 1.00 | 12.65 | | | C |
| ANISOU | 1126 | C | ALA | A | 523 | 1672 | 1298 | 1836 | −222 | −41 | −159 | | C |
| ATOM | 1127 | O | ALA | A | 523 | −15.249 | −17.766 | 17.661 | 1.00 | 9.28 | | | O |
| ANISOU | 1127 | O | ALA | A | 523 | 1268 | 878 | 1381 | −223 | −10 | −68 | | O |
| ATOM | 1128 | N | ALA | A | 524 | −16.457 | −16.809 | 19.321 | 1.00 | 8.13 | | | N |
| ANISOU | 1128 | N | ALA | A | 524 | 1119 | 707 | 1265 | −202 | −51 | −190 | | N |
| ATOM | 1129 | CA | ALA | A | 524 | −17.677 | −16.876 | 18.511 | 1.00 | 6.89 | | | C |
| ANISOU | 1129 | CA | ALA | A | 524 | 1008 | 529 | 1081 | −184 | −26 | −100 | | C |
| ATOM | 1130 | CB | ALA | A | 524 | −18.891 | −16.355 | 19.280 | 1.00 | 7.63 | | | C |
| ANISOU | 1130 | CB | ALA | A | 524 | 1108 | 614 | 1179 | −155 | −38 | −149 | | C |
| ATOM | 1131 | C | ALA | A | 524 | −17.528 | −16.087 | 17.224 | 1.00 | 7.26 | | | C |
| ANISOU | 1131 | C | ALA | A | 524 | 1042 | 503 | 1212 | −202 | 18 | −4 | | C |
| ATOM | 1132 | O | ALA | A | 524 | −18.138 | −16.437 | 16.214 | 1.00 | 9.61 | | | O |
| ANISOU | 1132 | O | ALA | A | 524 | 1367 | 836 | 1450 | −181 | 36 | 87 | | O |
| ATOM | 1133 | N | ARG | A | 525 | −16.743 | −15.012 | 17.245 | 1.00 | 8.15 | | | N |
| ANISOU | 1133 | N | ARG | A | 525 | 1104 | 524 | 1469 | −237 | 39 | −20 | | N |
| ATOM | 1134 | CA | ARG | A | 525 | −16.561 | −14.226 | 16.033 | 1.00 | 8.85 | | | C |
| ANISOU | 1134 | CA | ARG | A | 525 | 1169 | 542 | 1651 | −248 | 99 | 115 | | C |
| ATOM | 1135 | CB | ARG | A | 525 | −15.730 | −12.972 | 16.315 | 1.00 | 18.16 | | | C |
| ANISOU | 1135 | CB | ARG | A | 525 | 2280 | 1629 | 2991 | −281 | 117 | 75 | | C |
| ATOM | 1136 | CG | ARG | A | 525 | −14.246 | −13.262 | 16.497 | 1.00 | 23.28 | | | C |
| ANISOU | 1136 | CG | ARG | A | 525 | 2875 | 2302 | 3667 | −330 | 115 | 23 | | C |
| ATOM | 1137 | CD | ARG | A | 525 | −13.391 | −12.028 | 16.768 | 1.00 | 33.54 | | | C |
| ANISOU | 1137 | CD | ARG | A | 525 | 4093 | 3520 | 5129 | −362 | 130 | −25 | | C |
| ATOM | 1138 | NE | ARG | A | 525 | −12.182 | −12.434 | 17.486 | 1.00 | 39.43 | | | N |
| ANISOU | 1138 | NE | ARG | A | 525 | 4784 | 4315 | 5881 | −401 | 95 | −146 | | N |
| ATOM | 1139 | CZ | ARG | A | 525 | −12.077 | −12.452 | 18.811 | 1.00 | 32.14 | | | C |
| ANISOU | 1139 | CZ | ARG | A | 525 | 3837 | 3433 | 4944 | −394 | 30 | −321 | | C |
| ATOM | 1140 | NH1 | ARG | A | 525 | −13.089 | −12.054 | 19.558 | 1.00 | 27.57 | | | N |
| ANISOU | 1140 | NH1 | ARG | A | 525 | 3282 | 2842 | 4353 | −356 | 2 | −396 | | N |
| ATOM | 1141 | NH2 | ARG | A | 525 | −10.959 | −12.859 | 19.390 | 1.00 | 27.12 | | | N |
| ANISOU | 1141 | NH2 | ARG | A | 525 | 3141 | 2871 | 4294 | −415 | −7 | −420 | | N |
| ATOM | 1142 | C | ARG | A | 525 | −15.898 | −15.029 | 14.915 | 1.00 | 14.81 | | | C |
| ANISOU | 1142 | C | ARG | A | 525 | 1926 | 1393 | 2308 | −247 | 129 | 212 | | C |
| ATOM | 1143 | O | ARG | A | 525 | −16.016 | −14.637 | 13.750 | 1.00 | 13.96 | | | O |
| ANISOU | 1143 | O | ARG | A | 525 | 1806 | 1294 | 2204 | −229 | 181 | 354 | | O |
| ATOM | 1144 | N | ASN | A | 526 | −15.206 | −16.130 | 15.250 | 1.00 | 10.87 | | | N |
| ANISOU | 1144 | N | ASN | A | 526 | 1434 | 978 | 1718 | −252 | 99 | 142 | | N |
| ATOM | 1145 | CA | ASN | A | 526 | −14.496 | −16.994 | 14.306 | 1.00 | 16.88 | | | C |
| ANISOU | 1145 | CA | ASN | A | 526 | 2189 | 1833 | 2390 | −242 | 122 | 193 | | C |
| ATOM | 1146 | CB | ASN | A | 526 | −13.156 | −17.466 | 14.911 | 1.00 | 23.54 | | | C |
| ANISOU | 1146 | CB | ASN | A | 526 | 2992 | 2699 | 3252 | −267 | 106 | 114 | | C |
| ATOM | 1147 | CG | ASN | A | 526 | −12.192 | −16.342 | 15.092 | 1.00 | 34.37 | | | C |
| ANISOU | 1147 | CG | ASN | A | 526 | 4285 | 3991 | 4782 | −323 | 134 | 111 | | C |
| ATOM | 1148 | OD1 | ASN | A | 526 | −12.207 | −15.377 | 14.328 | 1.00 | 52.95 | | | O |
| ANISOU | 1148 | OD1 | ASN | A | 526 | 6607 | 6280 | 7233 | −344 | 195 | 217 | | O |
| ATOM | 1149 | ND2 | ASN | A | 526 | −11.346 | −16.441 | 16.095 | 1.00 | 23.07 | | | N |
| ANISOU | 1149 | ND2 | ASN | A | 526 | 2808 | 2567 | 3390 | −346 | 93 | −5 | | N |
| ATOM | 1150 | C | ASN | A | 526 | −15.279 | −18.234 | 13.904 | 1.00 | 14.65 | | | C |
| ANISOU | 1150 | C | ASN | A | 526 | 1959 | 1644 | 1962 | −199 | 98 | 182 | | C |
| ATOM | 1151 | O | ASN | A | 526 | −14.950 | −18.846 | 12.882 | 1.00 | 24.26 | | | O |
| ANISOU | 1151 | O | ASN | A | 526 | 3167 | 2948 | 3102 | −177 | 120 | 215 | | O |
| ATOM | 1152 | N | CYS | A | 527 | −16.282 | −18.632 | 14.678 | 1.00 | 10.07 | | | N |
| ANISOU | 1152 | N | CYS | A | 527 | 1420 | 1054 | 1352 | −186 | 56 | 126 | | N |
| ATOM | 1153 | CA | CYS | A | 527 | −17.123 | −19.745 | 14.271 | 1.00 | 6.18 | | | C |
| ANISOU | 1153 | CA | CYS | A | 527 | 959 | 623 | 765 | −158 | 38 | 111 | | C |
| ATOM | 1154 | CB | CYS | A | 527 | −17.808 | −20.367 | 15.491 | 1.00 | 5.68 | | | C |
| ANISOU | 1154 | CB | CYS | A | 527 | 923 | 537 | 697 | −157 | 3 | 52 | | C |
| ATOM | 1155 | SG | CYS | A | 527 | −16.610 | −20.912 | 16.725 | 1.00 | 11.87 | | | S |
| ANISOU | 1155 | SG | CYS | A | 527 | 1695 | 1319 | 1498 | −158 | −15 | 1 | | S |
| ATOM | 1156 | C | CYS | A | 527 | −18.154 | −19.243 | 13.270 | 1.00 | 6.50 | | | C |
| ANISOU | 1156 | C | CYS | A | 527 | 999 | 703 | 769 | −133 | 50 | 177 | | C |
| ATOM | 1157 | O | CYS | A | 527 | −18.577 | −18.083 | 13.321 | 1.00 | 9.76 | | | O |
| ANISOU | 1157 | O | CYS | A | 527 | 1403 | 1064 | 1240 | −130 | 65 | 236 | | O |
| ATOM | 1158 | N | LEU | A | 528 | −18.549 | −20.122 | 12.352 | 1.00 | 8.49 | | | N |
| ANISOU | 1158 | N | LEU | A | 528 | 1248 | 1049 | 929 | −109 | 41 | 159 | | N |
| ATOM | 1159 | CA | LEU | A | 528 | −19.468 | −19.786 | 11.273 | 1.00 | 7.17 | | | C |
| ANISOU | 1159 | CA | LEU | A | 528 | 1062 | 974 | 690 | −69 | 43 | 212 | | C |
| ATOM | 1160 | CB | LEU | A | 528 | −18.767 | −19.844 | 9.901 | 1.00 | 8.01 | | | C |
| ANISOU | 1160 | CB | LEU | A | 528 | 1128 | 1213 | 703 | −32 | 76 | 260 | | C |
| ATOM | 1161 | CG | LEU | A | 528 | −17.544 | −18.926 | 9.769 | 1.00 | 16.36 | | | C |
| ANISOU | 1161 | CG | LEU | A | 528 | 2162 | 2234 | 1821 | −44 | 137 | 368 | | C |
| ATOM | 1162 | CD1 | LEU | A | 528 | −16.790 | −19.160 | 8.467 | 1.00 | 10.32 | | | C |
| ANISOU | 1162 | CD1 | LEU | A | 528 | 1348 | 1630 | 945 | −2 | 180 | 416 | | C |
| ATOM | 1163 | CD2 | LEU | A | 528 | −18.036 | −17.494 | 9.821 | 1.00 | 15.50 | | | C |
| ANISOU | 1163 | CD2 | LEU | A | 528 | 2044 | 2051 | 1794 | −36 | 165 | 502 | | C |
| ATOM | 1164 | C | LEU | A | 528 | −20.635 | −20.757 | 11.300 | 1.00 | 11.33 | | | C |
| ANISOU | 1164 | C | LEU | A | 528 | 1592 | 1540 | 1175 | −65 | −3 | 121 | | C |
| ATOM | 1165 | O | LEU | A | 528 | −20.548 | −21.824 | 11.907 | 1.00 | 8.58 | | | O |
| ANISOU | 1165 | O | LEU | A | 528 | 1257 | 1145 | 856 | −93 | −20 | 33 | | O |
| ATOM | 1166 | N | VAL | A | 529 | −21.731 | −20.369 | 10.638 | 1.00 | 9.74 | | | N |
| ANISOU | 1166 | N | VAL | A | 529 | 1364 | 1419 | 916 | −29 | −18 | 153 | | N |
| ATOM | 1167 | CA | VAL | A | 529 | −22.979 | −21.130 | 10.618 | 1.00 | 9.30 | | | C |
| ANISOU | 1167 | CA | VAL | A | 529 | 1289 | 1406 | 838 | −32 | −63 | 64 | | C |
| ATOM | 1168 | CB | VAL | A | 529 | −24.096 | −20.393 | 11.384 | 1.00 | 11.98 | | | C |
| ANISOU | 1168 | CB | VAL | A | 529 | 1633 | 1689 | 1229 | −30 | −71 | 107 | | C |
| ATOM | 1169 | CG1 | VAL | A | 529 | −25.335 | −21.294 | 11.488 | 1.00 | 12.38 | | | C |
| ANISOU | 1169 | CG1 | VAL | A | 529 | 1649 | 1775 | 1280 | −49 | −110 | 11 | | C |
| ATOM | 1170 | CG2 | VAL | A | 529 | −23.595 | −19.970 | 12.785 | 1.00 | 6.60 | | | C |
| ANISOU | 1170 | CG2 | VAL | A | 529 | 998 | 864 | 648 | −64 | −48 | 123 | | C |
| ATOM | 1171 | C | VAL | A | 529 | −23.401 | −21.372 | 9.174 | 1.00 | 8.58 | | | C |
| ANISOU | 1171 | C | VAL | A | 529 | 1138 | 1503 | 618 | 20 | −87 | 36 | | C |
| ATOM | 1172 | O | VAL | A | 529 | −23.474 | −20.427 | 8.381 | 1.00 | 9.29 | | | O |
| ANISOU | 1172 | O | VAL | A | 529 | 1203 | 1695 | 632 | 81 | −71 | 152 | | O |
| ATOM | 1173 | N | ASN | A | 530 | −23.684 | −22.631 | 8.830 | 1.00 | 11.42 | | | N |
| ANISOU | 1173 | N | ASN | A | 530 | 1465 | 1915 | 960 | 3 | −123 | −119 | | N |
| ATOM | 1174 | CA | ASN | A | 530 | −24.065 | −22.961 | 7.464 | 1.00 | 10.17 | | | C |
| ANISOU | 1174 | CA | ASN | A | 530 | 1233 | 1968 | 665 | 57 | −159 | −198 | | C |
| ATOM | 1175 | CB | ASN | A | 530 | −23.508 | −24.332 | 7.063 | 1.00 | 11.07 | | | C |
| ANISOU | 1175 | CB | ASN | A | 530 | 1319 | 2101 | 786 | 38 | −174 | −385 | | C |
| ATOM | 1176 | CG | ASN | A | 530 | −24.303 | −25.502 | 7.634 | 1.00 | 14.89 | | | C |
| ANISOU | 1176 | CG | ASN | A | 530 | 1780 | 2471 | 1406 | −31 | −210 | −544 | | C |
| ATOM | 1177 | OD1 | ASN | A | 530 | −25.277 | −25.328 | 8.356 | 1.00 | 12.47 | | | O |
| ANISOU | 1177 | OD1 | ASN | A | 530 | 1476 | 2089 | 1174 | −67 | −221 | −510 | | O |
| ATOM | 1178 | ND2 | ASN | A | 530 | −23.880 | −26.708 | 7.293 | 1.00 | 11.81 | | | N |
| ANISOU | 1178 | ND2 | ASN | A | 530 | 1360 | 2051 | 1078 | −44 | −215 | −700 | | N |
| ATOM | 1179 | C | ASN | A | 530 | −25.587 | −22.866 | 7.307 | 1.00 | 17.01 | | | C |
| ANISOU | 1179 | C | ASN | A | 530 | 2043 | 2914 | 1508 | 70 | −211 | −233 | | C |
| ATOM | 1180 | O | ASN | A | 530 | −26.310 | −22.538 | 8.246 | 1.00 | 14.13 | | | O |
| ANISOU | 1180 | O | ASN | A | 530 | 1700 | 2433 | 1237 | 37 | −210 | −187 | | O |
| ATOM | 1181 | N | ASP | A | 531 | −26.082 | −23.140 | 6.097 | 1.00 | 12.08 | | | N |
| ANISOU | 1181 | N | ASP | A | 531 | 1333 | 2486 | 770 | 124 | −248 | −314 | | N |
| ATOM | 1182 | CA | ASP | A | 531 | −27.499 | −22.954 | 5.804 | 1.00 | 14.50 | | | C |
| ANISOU | 1182 | CA | ASP | A | 531 | 1569 | 2879 | 1060 | 148 | −290 | −333 | | C |
| ATOM | 1183 | CB | ASP | A | 531 | −27.785 | −23.170 | 4.305 | 1.00 | 16.65 | | | C |
| ANISOU | 1183 | CB | ASP | A | 531 | 1745 | 3356 | 1226 | 222 | −303 | −396 | | C |
| ATOM | 1184 | CG | ASP | A | 531 | −27.354 | −24.553 | 3.816 | 1.00 | 28.82 | | | C |
| ANISOU | 1184 | CG | ASP | A | 531 | 3244 | 4902 | 2804 | 193 | −314 | −604 | | C |
| ATOM | 1185 | OD1 | ASP | A | 531 | −26.318 | −25.083 | 4.281 | 1.00 | 32.84 | | | O |
| ANISOU | 1185 | OD1 | ASP | A | 531 | 3812 | 5287 | 3380 | 148 | −290 | −637 | | O |
| ATOM | 1186 | OD2 | ASP | A | 531 | −28.047 | −25.116 | 2.948 | 1.00 | 36.95 | | | O |
| ANISOU | 1186 | OD2 | ASP | A | 531 | 4174 | 6064 | 3803 | 222 | −346 | −739 | | O |
| ATOM | 1187 | C | ASP | A | 531 | −28.387 | −23.885 | 6.606 | 1.00 | 17.77 | | | C |
| ANISOU | 1187 | C | ASP | A | 531 | 1959 | 3185 | 1607 | 61 | −328 | −482 | | C |
| ATOM | 1188 | O | ASP | A | 531 | −29.597 | −23.656 | 6.652 | 1.00 | 18.93 | | | O |
| ANISOU | 1188 | O | ASP | A | 531 | 2051 | 3381 | 1762 | 67 | −359 | −486 | | O |
| ATOM | 1189 | N | GLN | A | 532 | −27.822 | −24.941 | 7.196 | 1.00 | 12.82 | | | N |
| ANISOU | 1189 | N | GLN | A | 532 | 1362 | 2407 | 1104 | −18 | −316 | −592 | | N |
| ATOM | 1190 | CA | GLN | A | 532 | −28.561 | −25.867 | 8.044 | 1.00 | 17.72 | | | C |
| ANISOU | 1190 | CA | GLN | A | 532 | 1956 | 2876 | 1899 | −109 | −324 | −693 | | C |
| ATOM | 1191 | CB | GLN | A | 532 | −28.089 | −27.298 | 7.783 | 1.00 | 16.59 | | | C |
| ANISOU | 1191 | CB | GLN | A | 532 | 1794 | 2636 | 1875 | −145 | −309 | −836 | | C |
| ATOM | 1192 | CG | GLN | A | 532 | −28.296 | −27.743 | 6.332 | 1.00 | 21.68 | | | C |
| ANISOU | 1192 | CG | GLN | A | 532 | 2349 | 3448 | 2441 | −87 | −335 | −961 | | C |
| ATOM | 1193 | CD | GLN | A | 532 | −29.753 | −27.713 | 5.922 | 1.00 | 28.87 | | | C |
| ANISOU | 1193 | CD | GLN | A | 532 | 3163 | 4464 | 3341 | −82 | −375 | −1024 | | C |
| ATOM | 1194 | OE1 | GLN | A | 532 | −30.583 | −28.402 | 6.508 | 1.00 | 30.97 | | | O |
| ANISOU | 1194 | OE1 | GLN | A | 532 | 3391 | 4613 | 3762 | −154 | −377 | −1090 | | O |
| ATOM | 1195 | NE2 | GLN | A | 532 | −30.074 | −26.902 | 4.921 | 1.00 | 27.46 | | | N |
| ANISOU | 1195 | NE2 | GLN | A | 532 | 2939 | 4509 | 2985 | 8 | −399 | −988 | | N |
| ATOM | 1196 | C | GLN | A | 532 | −28.433 | −25.538 | 9.527 | 1.00 | 19.63 | | | C |
| ANISOU | 1196 | C | GLN | A | 532 | 2280 | 2926 | 2252 | −157 | −273 | −559 | | C |
| ATOM | 1197 | O | GLN | A | 532 | −28.965 | −26.271 | 10.369 | 1.00 | 17.83 | | | O |
| ANISOU | 1197 | O | GLN | A | 532 | 2034 | 2565 | 2174 | −229 | −257 | −593 | | O |
| ATOM | 1198 | N | GLY | A | 533 | −27.750 | −24.455 | 9.867 | 1.00 | 12.70 | | | N |
| ANISOU | 1198 | N | GLY | A | 533 | 1483 | 2027 | 1317 | −115 | −236 | −396 | | N |
| ATOM | 1199 | CA | GLY | A | 533 | −27.593 | −24.092 | 11.254 | 1.00 | 9.81 | | | C |
| ANISOU | 1199 | CA | GLY | A | 533 | 1185 | 1504 | 1040 | −144 | −189 | −287 | | C |
| ATOM | 1200 | C | GLY | A | 533 | −26.473 | −24.814 | 11.963 | 1.00 | 12.20 | | | C |
| ANISOU | 1200 | C | GLY | A | 533 | 1543 | 1669 | 1422 | −181 | −152 | −289 | | C |
| ATOM | 1201 | O | GLY | A | 533 | −26.360 | −24.686 | 13.189 | 1.00 | 13.04 | | | O |
| ANISOU | 1201 | O | GLY | A | 533 | 1692 | 1672 | 1591 | −201 | −118 | −213 | | O |
| ATOM | 1202 | N | VAL | A | 534 | −25.655 | −25.578 | 11.236 | 1.00 | 12.51 | | | N |
| ANISOU | 1202 | N | VAL | A | 534 | 1574 | 1724 | 1454 | −180 | −159 | −377 | | N |
| ATOM | 1203 | CA | VAL | A | 534 | −24.515 | −26.278 | 11.824 | 1.00 | 10.27 | | | C |
| ANISOU | 1203 | CA | VAL | A | 534 | 1336 | 1319 | 1249 | −198 | −125 | −374 | | C |
| ATOM | 1204 | CB | VAL | A | 534 | −24.089 | −27.466 | 10.946 | 1.00 | 10.71 | | | C |
| ANISOU | 1204 | CB | VAL | A | 534 | 1348 | 1383 | 1341 | −201 | −141 | −531 | | C |
| ATOM | 1205 | CG1 | VAL | A | 534 | −22.824 | −28.151 | 11.537 | 1.00 | 10.20 | | | C |
| ANISOU | 1205 | CG1 | VAL | A | 534 | 1325 | 1190 | 1361 | −201 | −103 | −511 | | C |
| ATOM | 1206 | CG2 | VAL | A | 534 | −25.209 | −28.455 | 10.805 | 1.00 | 15.33 | | | C |
| ANISOU | 1206 | CG2 | VAL | A | 534 | 1855 | 1930 | 2041 | −252 | −166 | −664 | | C |
| ATOM | 1207 | C | VAL | A | 534 | −23.368 | −25.289 | 11.991 | 1.00 | 15.10 | | | C |
| ANISOU | 1207 | C | VAL | A | 534 | 2007 | 1942 | 1789 | −161 | −101 | −265 | | C |
| ATOM | 1208 | O | VAL | A | 534 | −23.075 | −24.506 | 11.079 | 1.00 | 8.49 | | | O |
| ANISOU | 1208 | O | VAL | A | 534 | 1162 | 1217 | 846 | −119 | −105 | −236 | | O |
| ATOM | 1209 | N | VAL | A | 535 | −22.732 | −25.309 | 13.166 | 1.00 | 8.96 | | | N |
| ANISOU | 1209 | N | VAL | A | 535 | 1275 | 1059 | 1071 | −175 | −72 | −197 | | N |
| ATOM | 1210 | CA | VAL | A | 535 | −21.620 | −24.417 | 13.487 | 1.00 | 10.11 | | | C |
| ANISOU | 1210 | CA | VAL | A | 535 | 1459 | 1201 | 1181 | −154 | −53 | −120 | | C |
| ATOM | 1211 | CB | VAL | A | 535 | −21.632 | −24.034 | 14.981 | 1.00 | 10.74 | | | C |
| ANISOU | 1211 | CB | VAL | A | 535 | 1567 | 1213 | 1301 | −163 | −39 | −55 | | C |
| ATOM | 1212 | CG1 | VAL | A | 535 | −20.454 | −23.136 | 15.281 | 1.00 | 10.13 | | | C |
| ANISOU | 1212 | CG1 | VAL | A | 535 | 1509 | 1133 | 1208 | −149 | −29 | −15 | | C |
| ATOM | 1213 | CG2 | VAL | A | 535 | −22.931 | −23.344 | 15.336 | 1.00 | 11.17 | | | C |
| ANISOU | 1213 | CG2 | VAL | A | 535 | 1613 | 1284 | 1349 | −164 | −45 | −30 | | C |
| ATOM | 1214 | C | VAL | A | 535 | −20.319 | −25.110 | 13.127 | 1.00 | 9.62 | | | C |
| ANISOU | 1214 | C | VAL | A | 535 | 1397 | 1131 | 1126 | −142 | −41 | −158 | | C |
| ATOM | 1215 | O | VAL | A | 535 | −20.139 | −26.299 | 13.423 | 1.00 | 8.78 | | | O |
| ANISOU | 1215 | O | VAL | A | 535 | 1284 | 955 | 1096 | −150 | −37 | −208 | | O |
| ATOM | 1216 | N | LYS | A | 536 | −19.393 | −24.372 | 12.497 | 1.00 | 7.90 | | | N |
| ANISOU | 1216 | N | LYS | A | 536 | 1178 | 977 | 848 | −119 | −27 | −125 | | N |
| ATOM | 1217 | CA | LYS | A | 536 | −18.077 | −24.921 | 12.176 | 1.00 | 10.39 | | | C |
| ANISOU | 1217 | CA | LYS | A | 536 | 1483 | 1301 | 1164 | −101 | −10 | −157 | | C |
| ATOM | 1218 | CB | LYS | A | 536 | −17.941 | −25.258 | 10.676 | 1.00 | 10.06 | | | C |
| ANISOU | 1218 | CB | LYS | A | 536 | 1400 | 1385 | 1039 | −67 | −8 | −230 | | C |
| ATOM | 1219 | CG | LYS | A | 536 | −18.999 | −26.241 | 10.172 | 1.00 | 9.76 | | | C |
| ANISOU | 1219 | CG | LYS | A | 536 | 1331 | 1365 | 1012 | −70 | −42 | −359 | | C |
| ATOM | 1220 | CD | LYS | A | 536 | −18.651 | −26.809 | 8.814 | 1.00 | 9.65 | | | C |
| ANISOU | 1220 | CD | LYS | A | 536 | 1262 | 1489 | 916 | −25 | −47 | −485 | | C |
| ATOM | 1221 | CE | LYS | A | 536 | −19.865 | −27.514 | 8.223 | 1.00 | 10.13 | | | C |
| ANISOU | 1221 | CE | LYS | A | 536 | 1272 | 1599 | 980 | −31 | −94 | −636 | | C |
| ATOM | 1222 | NZ | LYS | A | 536 | −19.495 | −28.369 | 7.072 | 1.00 | 12.05 | | | N |
| ANISOU | 1222 | NZ | LYS | A | 536 | 1449 | 1949 | 1182 | 14 | −104 | −802 | | N |
| ATOM | 1223 | C | LYS | A | 536 | −16.984 | −23.937 | 12.576 | 1.00 | 12.36 | | | C |
| ANISOU | 1223 | C | LYS | A | 536 | 1739 | 1544 | 1414 | −103 | 14 | −75 | | C |
| ATOM | 1224 | O | LYS | A | 536 | −17.200 | −22.724 | 12.617 | 1.00 | 9.62 | | | O |
| ANISOU | 1224 | O | LYS | A | 536 | 1395 | 1203 | 1059 | −112 | 23 | −3 | | O |
| ATOM | 1225 | N | VAL | A | 537 | −15.809 | −24.467 | 12.864 | 1.00 | 10.29 | | | N |
| ANISOU | 1225 | N | VAL | A | 537 | 1466 | 1262 | 1182 | −93 | 24 | −94 | | N |
| ATOM | 1226 | CA | VAL | A | 537 | −14.683 | −23.650 | 13.315 | 1.00 | 8.89 | | | C |
| ANISOU | 1226 | CA | VAL | A | 537 | 1273 | 1081 | 1024 | −102 | 40 | −42 | | C |
| ATOM | 1227 | CB | VAL | A | 537 | −13.766 | −24.485 | 14.231 | 1.00 | 9.62 | | | C |
| ANISOU | 1227 | CB | VAL | A | 537 | 1357 | 1134 | 1162 | −85 | 29 | −68 | | C |
| ATOM | 1228 | CG1 | VAL | A | 537 | −12.391 | −23.839 | 14.427 | 1.00 | 11.48 | | | C |
| ANISOU | 1228 | CG1 | VAL | A | 537 | 1547 | 1398 | 1415 | −91 | 42 | −49 | | C |
| ATOM | 1229 | CG2 | VAL | A | 537 | −14.442 | −24.723 | 15.579 | 1.00 | 9.21 | | | C |
| ANISOU | 1229 | CG2 | VAL | A | 537 | 1335 | 1025 | 1140 | −87 | 2 | −52 | | C |
| ATOM | 1230 | C | VAL | A | 537 | −13.948 | −23.123 | 12.085 | 1.00 | 9.11 | | | C |
| ANISOU | 1230 | C | VAL | A | 537 | 1259 | 1198 | 1004 | −92 | 81 | −11 | | C |
| ATOM | 1231 | O | VAL | A | 537 | −13.759 | −23.847 | 11.106 | 1.00 | 8.37 | | | O |
| ANISOU | 1231 | O | VAL | A | 537 | 1144 | 1183 | 854 | −57 | 93 | −61 | | O |
| ATOM | 1232 | N | SER | A | 538 | −13.569 | −21.845 | 12.106 | 1.00 | 9.63 | | | N |
| ANISOU | 1232 | N | SER | A | 538 | 1303 | 1255 | 1100 | −118 | 107 | 70 | | N |
| ATOM | 1233 | CA | SER | A | 538 | −12.844 | −21.267 | 10.981 | 1.00 | 8.92 | | | C |
| ANISOU | 1233 | CA | SER | A | 538 | 1161 | 1251 | 978 | −109 | 165 | 143 | | C |
| ATOM | 1234 | CB | SER | A | 538 | −13.758 | −20.417 | 10.090 | 1.00 | 16.50 | | | C |
| ANISOU | 1234 | CB | SER | A | 538 | 2119 | 2262 | 1887 | −93 | 190 | 242 | | C |
| ATOM | 1235 | OG | SER | A | 538 | −13.869 | −19.109 | 10.603 | 1.00 | 17.58 | | | O |
| ANISOU | 1235 | OG | SER | A | 538 | 2252 | 2294 | 2134 | −132 | 207 | 329 | | O |
| ATOM | 1236 | C | SER | A | 538 | −11.683 | −20.417 | 11.491 | 1.00 | 10.33 | | | C |
| ANISOU | 1236 | C | SER | A | 538 | 1290 | 1375 | 1259 | −153 | 193 | 186 | | C |
| ATOM | 1237 | O | SER | A | 538 | −11.498 | −20.222 | 12.694 | 1.00 | 13.40 | | | O |
| ANISOU | 1237 | O | SER | A | 538 | 1685 | 1678 | 1729 | −185 | 157 | 139 | | O |
| ATOM | 1238 | N | ASP | A | 539 | −10.898 | −19.912 | 10.544 | 1.00 | 11.54 | | | N |
| ANISOU | 1238 | N | ASP | A | 539 | 1380 | 1598 | 1406 | −152 | 261 | 272 | | N |
| ATOM | 1239 | CA | ASP | A | 539 | −9.747 | −19.069 | 10.831 | 1.00 | 10.83 | | | C |
| ANISOU | 1239 | CA | ASP | A | 539 | 1217 | 1459 | 1439 | −205 | 301 | 317 | | C |
| ATOM | 1240 | CB | ASP | A | 539 | −10.184 | −17.767 | 11.508 | 1.00 | 12.48 | | | C |
| ANISOU | 1240 | CB | ASP | A | 539 | 1426 | 1524 | 1793 | −262 | 299 | 359 | | C |
| ATOM | 1241 | CG | ASP | A | 539 | −10.542 | −16.685 | 10.503 | 1.00 | 31.17 | | | C |
| ANISOU | 1241 | CG | ASP | A | 539 | 3765 | 3885 | 4195 | −261 | 376 | 533 | | C |
| ATOM | 1242 | OD1 | ASP | A | 539 | −10.153 | −16.825 | 9.323 | 1.00 | 38.44 | | | O |
| ANISOU | 1242 | OD1 | ASP | A | 539 | 4645 | 4936 | 5026 | −224 | 442 | 636 | | O |
| ATOM | 1243 | OD2 | ASP | A | 539 | −11.194 | −15.693 | 10.886 | 1.00 | 31.87 | | | O |
| ANISOU | 1243 | OD2 | ASP | A | 539 | 3863 | 3846 | 4399 | −286 | 376 | 573 | | O |
| ATOM | 1244 | C | ASP | A | 539 | −8.697 | −19.771 | 11.684 | 1.00 | 12.79 | | | C |
| ANISOU | 1244 | C | ASP | A | 539 | 1436 | 1702 | 1722 | −213 | 265 | 208 | | C |
| ATOM | 1245 | O | ASP | A | 539 | −7.892 | −19.109 | 12.340 | 1.00 | 10.85 | | | O |
| ANISOU | 1245 | O | ASP | A | 539 | 1128 | 1398 | 1594 | −266 | 265 | 195 | | O |
| ATOM | 1246 | N | PHE | A | 540 | −8.665 | −21.096 | 11.694 | 1.00 | 12.41 | | | N |
| ANISOU | 1246 | N | PHE | A | 540 | 1418 | 1711 | 1586 | −157 | 232 | 125 | | N |
| ATOM | 1247 | CA | PHE | A | 540 | −7.834 | −21.789 | 12.667 | 1.00 | 11.35 | | | C |
| ANISOU | 1247 | CA | PHE | A | 540 | 1262 | 1564 | 1487 | −146 | 188 | 41 | | C |
| ATOM | 1248 | CB | PHE | A | 540 | −8.208 | −23.276 | 12.722 | 1.00 | 13.93 | | | C |
| ANISOU | 1248 | CB | PHE | A | 540 | 1640 | 1903 | 1748 | −78 | 154 | −30 | | C |
| ATOM | 1249 | CG | PHE | A | 540 | −8.644 | −23.860 | 11.400 | 1.00 | 19.33 | | | C |
| ANISOU | 1249 | CG | PHE | A | 540 | 2338 | 2661 | 2344 | −35 | 187 | −44 | | C |
| ATOM | 1250 | CD1 | PHE | A | 540 | −9.970 | −23.764 | 10.984 | 1.00 | 19.17 | | | C |
| ANISOU | 1250 | CD1 | PHE | A | 540 | 2374 | 2636 | 2275 | −38 | 177 | −36 | | C |
| ATOM | 1251 | CE1 | PHE | A | 540 | −10.380 | −24.307 | 9.763 | 1.00 | 14.71 | | | C |
| ANISOU | 1251 | CE1 | PHE | A | 540 | 1803 | 2174 | 1613 | 8 | 195 | −78 | | C |
| ATOM | 1252 | CZ | PHE | A | 540 | −9.481 | −24.966 | 8.975 | 1.00 | 13.24 | | | C |
| ANISOU | 1252 | CZ | PHE | A | 540 | 1561 | 2090 | 1380 | 60 | 228 | −136 | | C |
| ATOM | 1253 | CE2 | PHE | A | 540 | −8.148 | −25.078 | 9.380 | 1.00 | 22.52 | | | C |
| ANISOU | 1253 | CE2 | PHE | A | 540 | 2685 | 3259 | 2612 | 65 | 248 | −133 | | C |
| ATOM | 1254 | CD2 | PHE | A | 540 | −7.743 | −24.529 | 10.593 | 1.00 | 17.97 | | | C |
| ANISOU | 1254 | CD2 | PHE | A | 540 | 2110 | 2584 | 2133 | 16 | 224 | −84 | | C |
| ATOM | 1255 | C | PHE | A | 540 | −6.346 | −21.587 | 12.366 | 1.00 | 15.34 | | | C |
| ANISOU | 1255 | C | PHE | A | 540 | 1662 | 2132 | 2036 | −157 | 232 | 53 | | C |
| ATOM | 1256 | O | PHE | A | 540 | −5.877 | −21.820 | 11.243 | 1.00 | 13.92 | | | O |
| ANISOU | 1256 | O | PHE | A | 540 | 1440 | 2048 | 1800 | −128 | 294 | 87 | | O |
| ATOM | 1257 | N | GLY | A | 541 | −5.607 | −21.120 | 13.375 | 1.00 | 12.14 | | | N |
| ANISOU | 1257 | N | GLY | A | 541 | 1197 | 1690 | 1725 | −198 | 201 | 15 | | N |
| ATOM | 1258 | CA | GLY | A | 541 | −4.176 | −20.921 | 13.281 | 1.00 | 11.25 | | | C |
| ANISOU | 1258 | CA | GLY | A | 541 | 966 | 1634 | 1675 | −217 | 231 | 8 | | C |
| ATOM | 1259 | C | GLY | A | 541 | −3.744 | −19.645 | 12.592 | 1.00 | 12.28 | | | C |
| ANISOU | 1259 | C | GLY | A | 541 | 1012 | 1745 | 1909 | −296 | 313 | 102 | | C |
| ATOM | 1260 | O | GLY | A | 541 | −2.540 | −19.346 | 12.566 | 1.00 | 13.30 | | | O |
| ANISOU | 1260 | O | GLY | A | 541 | 1021 | 1910 | 2121 | −332 | 345 | 99 | | O |
| ATOM | 1261 | N | LEU | A | 542 | −4.683 | −18.875 | 12.042 | 1.00 | 12.75 | | | N |
| ANISOU | 1261 | N | LEU | A | 542 | 1237 | 2414 | 1192 | −304 | 78 | −358 | | N |
| ATOM | 1262 | CA | LEU | A | 542 | −4.318 | −17.765 | 11.171 | 1.00 | 12.90 | | | C |
| ANISOU | 1262 | CA | LEU | A | 542 | 1258 | 2505 | 1137 | −135 | −5 | −222 | | C |
| ATOM | 1263 | CB | LEU | A | 542 | −5.551 | −17.269 | 10.407 | 1.00 | 15.91 | | | C |
| ANISOU | 1263 | CB | LEU | A | 542 | 1489 | 3266 | 1289 | −24 | −74 | −284 | | C |
| ATOM | 1264 | CG | LEU | A | 542 | −6.011 | −18.134 | 9.232 | 1.00 | 21.67 | | | C |
| ANISOU | 1264 | CG | LEU | A | 542 | 2079 | 4312 | 1844 | −146 | −34 | −485 | | C |
| ATOM | 1265 | CD1 | LEU | A | 542 | −6.941 | −17.311 | 8.370 | 1.00 | 22.33 | | | C |
| ANISOU | 1265 | CD1 | LEU | A | 542 | 1994 | 4794 | 1697 | 82 | −109 | −450 | | C |
| ATOM | 1266 | CD2 | LEU | A | 542 | −4.835 | −18.655 | 8.426 | 1.00 | 25.76 | | | C |
| ANISOU | 1266 | CD2 | LEU | A | 542 | 2703 | 4653 | 2432 | −220 | 49 | −457 | | C |
| ATOM | 1267 | C | LEU | A | 542 | −3.683 | −16.602 | 11.918 | 1.00 | 14.43 | | | C |
| ANISOU | 1267 | C | LEU | A | 542 | 1549 | 2500 | 1432 | −12 | −46 | −46 | | C |
| ATOM | 1268 | O | LEU | A | 542 | −3.005 | −15.782 | 11.286 | 1.00 | 13.90 | | | O |
| ANISOU | 1268 | O | LEU | A | 542 | 1555 | 2369 | 1359 | 87 | −30 | 71 | | O |
| ATOM | 1269 | N | SER | A | 543 | −3.880 | −16.499 | 13.238 | 1.00 | 14.27 | | | N |
| ANISOU | 1269 | N | SER | A | 543 | 1541 | 2381 | 1500 | −37 | −65 | −45 | | N |
| ATOM | 1270 | CA | SER | A | 543 | −3.261 | −15.391 | 13.960 | 1.00 | 12.52 | | | C |
| ANISOU | 1270 | CA | SER | A | 543 | 1399 | 2000 | 1358 | 20 | −71 | 64 | | C |
| ATOM | 1271 | CB | SER | A | 543 | −3.712 | −15.390 | 15.432 | 1.00 | 16.62 | | | C |
| ANISOU | 1271 | CB | SER | A | 543 | 1900 | 2478 | 1938 | −14 | −103 | 31 | | C |
| ATOM | 1272 | OG | SER | A | 543 | −3.303 | −16.568 | 16.098 | 1.00 | 14.59 | | | O |
| ANISOU | 1272 | OG | SER | A | 543 | 1621 | 2178 | 1745 | −102 | −71 | −11 | | O |
| ATOM | 1273 | C | SER | A | 543 | −1.738 | −15.427 | 13.875 | 1.00 | 12.60 | | | C |
| ANISOU | 1273 | C | SER | A | 543 | 1457 | 1874 | 1456 | −41 | −21 | 115 | | C |
| ATOM | 1274 | O | SER | A | 543 | −1.093 | −14.400 | 14.094 | 1.00 | 13.29 | | | O |
| ANISOU | 1274 | O | SER | A | 543 | 1603 | 1864 | 1582 | −42 | 17 | 160 | | O |
| ATOM | 1275 | N | ARG | A | 544 | −1.147 | −16.572 | 13.543 | 1.00 | 10.22 | | | N |
| ANISOU | 1275 | N | ARG | A | 544 | 1135 | 1573 | 1174 | −100 | 15 | 94 | | N |
| ATOM | 1276 | CA | ARG | A | 544 | 0.310 | −16.657 | 13.482 | 1.00 | 10.15 | | | C |
| ANISOU | 1276 | CA | ARG | A | 544 | 1136 | 1502 | 1219 | −126 | 61 | 151 | | C |
| ATOM | 1277 | CB | ARG | A | 544 | 0.747 | −18.114 | 13.319 | 1.00 | 11.03 | | | C |
| ANISOU | 1277 | CB | ARG | A | 544 | 1248 | 1606 | 1338 | −137 | 143 | 151 | | C |
| ATOM | 1278 | CG | ARG | A | 544 | 0.507 | −19.022 | 14.539 | 1.00 | 14.14 | | | C |
| ANISOU | 1278 | CG | ARG | A | 544 | 1636 | 1986 | 1752 | −115 | 198 | 146 | | C |
| ATOM | 1279 | CD | ARG | A | 544 | 0.755 | −20.514 | 14.176 | 1.00 | 14.16 | | | C |
| ANISOU | 1279 | CD | ARG | A | 544 | 1720 | 1909 | 1752 | −104 | 386 | 153 | | C |
| ATOM | 1280 | NE | ARG | A | 544 | −0.237 | −21.026 | 13.225 | 1.00 | 11.93 | | | N |
| ANISOU | 1280 | NE | ARG | A | 544 | 1480 | 1612 | 1440 | −228 | 453 | 12 | | N |
| ATOM | 1281 | CZ | ARG | A | 544 | −0.193 | −22.232 | 12.657 | 1.00 | 19.84 | | | C |
| ANISOU | 1281 | CZ | ARG | A | 544 | 2580 | 2520 | 2436 | −294 | 666 | −48 | | C |
| ATOM | 1282 | NH1 | ARG | A | 544 | 0.802 | −23.078 | 12.927 | 1.00 | 21.91 | | | N |
| ANISOU | 1282 | NH1 | ARG | A | 544 | 2944 | 2655 | 2727 | −186 | 851 | 77 | | N |
| ATOM | 1283 | NH2 | ARG | A | 544 | −1.149 | −22.598 | 11.811 | 1.00 | 17.14 | | | N |
| ANISOU | 1283 | NH2 | ARG | A | 544 | 2230 | 2245 | 2036 | −463 | 723 | −242 | | N |
| ATOM | 1284 | C | ARG | A | 544 | 0.897 | −15.820 | 12.350 | 1.00 | 14.90 | | | C |
| ANISOU | 1284 | C | ARG | A | 544 | 1792 | 2066 | 1802 | −108 | 93 | 198 | | C |
| ATOM | 1285 | O | ARG | A | 544 | 2.107 | −15.570 | 12.349 | 1.00 | 15.35 | | | O |
| ANISOU | 1285 | O | ARG | A | 544 | 1842 | 2089 | 1900 | −154 | 138 | 223 | | O |
| ATOM | 1286 | N | TYR | A | 545 | 0.080 | −15.391 | 11.382 | 1.00 | 12.14 | | | N |
| ANISOU | 1286 | N | TYR | A | 545 | 1482 | 1762 | 1369 | −24 | 88 | 212 | | N |
| ATOM | 1287 | CA | TYR | A | 545 | 0.584 | −14.738 | 10.176 | 1.00 | 13.64 | | | C |
| ANISOU | 1287 | CA | TYR | A | 545 | 1748 | 1915 | 1518 | 42 | 155 | 283 | | C |
| ATOM | 1288 | CB | TYR | A | 545 | 0.157 | −15.546 | 8.928 | 1.00 | 14.51 | | | C |
| ANISOU | 1288 | CB | TYR | A | 545 | 1818 | 2180 | 1517 | 82 | 138 | 256 | | C |
| ATOM | 1289 | CG | TYR | A | 545 | 0.463 | −17.005 | 9.181 | 1.00 | 15.81 | | | C |
| ANISOU | 1289 | CG | TYR | A | 545 | 1930 | 2342 | 1736 | −54 | 147 | 170 | | C |
| ATOM | 1290 | CD1 | TYR | A | 545 | 1.777 | −17.420 | 9.346 | 1.00 | 21.30 | | | C |
| ANISOU | 1290 | CD1 | TYR | A | 545 | 2650 | 2920 | 2525 | −105 | 205 | 216 | | C |
| ATOM | 1291 | CE1 | TYR | A | 545 | 2.086 | −18.734 | 9.657 | 1.00 | 19.01 | | | C |
| ANISOU | 1291 | CE1 | TYR | A | 545 | 2355 | 2598 | 2268 | −156 | 278 | 188 | | C |
| ATOM | 1292 | CZ | TYR | A | 545 | 1.083 | −19.645 | 9.830 | 1.00 | 15.42 | | | C |
| ANISOU | 1292 | CZ | TYR | A | 545 | 1900 | 2176 | 1783 | −217 | 325 | 78 | | C |
| ATOM | 1293 | OH | TYR | A | 545 | 1.421 | −20.941 | 10.145 | 1.00 | 18.17 | | | O |
| ANISOU | 1293 | OH | TYR | A | 545 | 2312 | 2422 | 2170 | −249 | 489 | 66 | | O |
| ATOM | 1294 | CE2 | TYR | A | 545 | −0.241 | −19.266 | 9.687 | 1.00 | 15.30 | | | C |
| ANISOU | 1294 | CE2 | TYR | A | 545 | 1827 | 2305 | 1682 | −234 | 250 | −22 | | C |
| ATOM | 1295 | CD2 | TYR | A | 545 | −0.548 | −17.939 | 9.379 | 1.00 | 16.23 | | | C |
| ANISOU | 1295 | CD2 | TYR | A | 545 | 1921 | 2507 | 1740 | −123 | 144 | 42 | | C |
| ATOM | 1296 | C | TYR | A | 545 | 0.153 | −13.278 | 10.094 | 1.00 | 16.27 | | | C |
| ANISOU | 1296 | C | TYR | A | 545 | 2196 | 2178 | 1809 | 170 | 232 | 361 | | C |
| ATOM | 1297 | O | TYR | A | 545 | 0.448 | −12.605 | 9.096 | 1.00 | 19.14 | | | O |
| ANISOU | 1297 | O | TYR | A | 545 | 2666 | 2485 | 2121 | 276 | 341 | 448 | | O |
| ATOM | 1298 | N | VAL | A | 546 | −0.471 | −12.759 | 11.145 | 1.00 | 14.82 | | | N |
| ANISOU | 1298 | N | VAL | A | 546 | 2022 | 1964 | 1647 | 178 | 222 | 344 | | N |
| ATOM | 1299 | CA | VAL | A | 546 | −0.922 | −11.371 | 11.188 | 1.00 | 15.91 | | | C |
| ANISOU | 1299 | CA | VAL | A | 546 | 2314 | 1986 | 1746 | 319 | 361 | 431 | | C |
| ATOM | 1300 | CB | VAL | A | 546 | −2.023 | −11.196 | 12.249 | 1.00 | 17.66 | | | C |
| ANISOU | 1300 | CB | VAL | A | 546 | 2499 | 2262 | 1949 | 364 | 299 | 406 | | C |
| ATOM | 1301 | CG1 | VAL | A | 546 | −2.373 | −9.716 | 12.411 | 1.00 | 19.88 | | | C |
| ANISOU | 1301 | CG1 | VAL | A | 546 | 2987 | 2363 | 2202 | 515 | 509 | 507 | | C |
| ATOM | 1302 | CG2 | VAL | A | 546 | −3.250 | −12.010 | 11.876 | 1.00 | 18.03 | | | C |
| ANISOU | 1302 | CG2 | VAL | A | 546 | 2389 | 2606 | 1855 | 481 | 149 | 386 | | C |
| ATOM | 1303 | C | VAL | A | 546 | 0.260 | −10.462 | 11.489 | 1.00 | 16.55 | | | C |
| ANISOU | 1303 | C | VAL | A | 546 | 2532 | 1814 | 1942 | 170 | 551 | 408 | | C |
| ATOM | 1304 | O | VAL | A | 546 | 0.958 | −10.648 | 12.492 | 1.00 | 16.15 | | | O |
| ANISOU | 1304 | O | VAL | A | 546 | 2400 | 1746 | 1990 | −50 | 521 | 288 | | O |
| ATOM | 1305 | N | LEU | A | 547 | 0.465 | −9.449 | 10.643 | 1.00 | 19.01 | | | N |
| ANISOU | 1305 | N | LEU | A | 547 | 3048 | 1959 | 2218 | 290 | 780 | 509 | | N |
| ATOM | 1306 | CA | LEU | A | 547 | 1.620 | −8.568 | 10.783 | 1.00 | 20.45 | | | C |
| ANISOU | 1306 | CA | LEU | A | 547 | 3377 | 1886 | 2506 | 92 | 1034 | 440 | | C |
| ATOM | 1307 | CB | LEU | A | 547 | 2.092 | −8.073 | 9.407 | 1.00 | 21.84 | | | C |
| ANISOU | 1307 | CB | LEU | A | 547 | 3734 | 1928 | 2638 | 222 | 1245 | 559 | | C |
| ATOM | 1308 | CG | LEU | A | 547 | 2.701 | −9.154 | 8.509 | 1.00 | 19.10 | | | C |
| ANISOU | 1308 | CG | LEU | A | 547 | 3230 | 1753 | 2274 | 200 | 1070 | 563 | | C |
| ATOM | 1309 | CD1 | LEU | A | 547 | 3.059 | −8.620 | 7.134 | 1.00 | 21.70 | | | C |
| ANISOU | 1309 | CD1 | LEU | A | 547 | 3746 | 1959 | 2541 | 364 | 1282 | 697 | | C |
| ATOM | 1310 | CD2 | LEU | A | 547 | 3.932 | −9.762 | 9.173 | 1.00 | 20.18 | | | C |
| ANISOU | 1310 | CD2 | LEU | A | 547 | 3193 | 1941 | 2532 | −124 | 986 | 389 | | C |
| ATOM | 1311 | C | LEU | A | 547 | 1.354 | −7.369 | 11.682 | 1.00 | 19.49 | | | C |
| ANISOU | 1311 | C | LEU | A | 547 | 3439 | 1539 | 2427 | 34 | 1273 | 397 | | C |
| ATOM | 1312 | O | LEU | A | 547 | 2.311 | −6.761 | 12.171 | 1.00 | 33.64 | | | O |
| ANISOU | 1312 | O | LEU | A | 547 | 5294 | 3173 | 4316 | −253 | 1476 | 236 | | O |
| ATOM | 1313 | N | ASP | A | 548 | 0.091 | −7.017 | 11.911 | 1.00 | 19.87 | | | N |
| ANISOU | 1313 | N | ASP | A | 548 | 3563 | 1596 | 2392 | 281 | 1277 | 512 | | N |
| ATOM | 1314 | CA | ASP | A | 548 | −0.258 | −5.891 | 12.780 | 1.00 | 32.42 | | | C |
| ANISOU | 1314 | CA | ASP | A | 548 | 5356 | 2945 | 4017 | 252 | 1536 | 485 | | C |
| ATOM | 1315 | CB | ASP | A | 548 | −1.737 | −5.551 | 12.626 | 1.00 | 36.29 | | | C |
| ANISOU | 1315 | CB | ASP | A | 548 | 5919 | 3509 | 4359 | 658 | 1538 | 681 | | C |
| ATOM | 1316 | CG | ASP | A | 548 | −2.122 | −4.277 | 13.352 | 1.00 | 42.97 | | | C |
| ANISOU | 1316 | CG | ASP | A | 548 | 6962 | 4135 | 5231 | 689 | 1820 | 625 | | C |
| ATOM | 1317 | OD1 | ASP | A | 548 | −1.335 | −3.801 | 14.201 | 1.00 | 41.81 | | | O |
| ANISOU | 1317 | OD1 | ASP | A | 548 | 6874 | 3803 | 5210 | 345 | 1971 | 411 | | O |
| ATOM | 1318 | OD2 | ASP | A | 548 | −3.217 | −3.750 | 13.066 | 1.00 | 48.60 | | | O |
| ANISOU | 1318 | OD2 | ASP | A | 548 | 7741 | 4918 | 5807 | 1057 | 1902 | 773 | | O |
| ATOM | 1319 | C | ASP | A | 548 | 0.064 | −6.236 | 14.231 | 1.00 | 29.22 | | | C |
| ANISOU | 1319 | C | ASP | A | 548 | 4767 | 2621 | 3713 | −80 | 1386 | 256 | | C |
| ATOM | 1320 | O | ASP | A | 548 | −0.575 | −7.110 | 14.824 | 1.00 | 25.71 | | | O |
| ANISOU | 1320 | O | ASP | A | 548 | 4111 | 2411 | 3246 | −39 | 1085 | 247 | | O |
| ATOM | 1321 | N | ASP | A | 549 | 1.043 | −5.542 | 14.818 | 1.00 | 31.15 | | | N |
| ANISOU | 1321 | N | ASP | A | 549 | 5082 | 2703 | 4050 | −415 | 1620 | 51 | | N |
| ATOM | 1322 | CA | ASP | A | 549 | 1.464 | −5.892 | 16.172 | 1.00 | 31.19 | | | C |
| ANISOU | 1322 | CA | ASP | A | 549 | 4861 | 2890 | 4101 | −721 | 1472 | −180 | | C |
| ATOM | 1323 | CB | ASP | A | 549 | 2.750 | −5.143 | 16.536 | 1.00 | 42.81 | | | C |
| ANISOU | 1323 | CB | ASP | A | 549 | 6309 | 4371 | 5587 | −1030 | 1657 | −468 | | C |
| ATOM | 1324 | CG | ASP | A | 549 | 3.975 | −5.678 | 15.792 | 1.00 | 50.01 | | | C |
| ANISOU | 1324 | CG | ASP | A | 549 | 7073 | 5430 | 6498 | −1139 | 1589 | −521 | | C |
| ATOM | 1325 | OD1 | ASP | A | 549 | 3.918 | −6.800 | 15.240 | 1.00 | 46.47 | | | O |
| ANISOU | 1325 | OD1 | ASP | A | 549 | 6490 | 5113 | 6053 | −1039 | 1373 | −364 | | O |
| ATOM | 1326 | OD2 | ASP | A | 549 | 5.005 | −4.972 | 15.764 | 1.00 | 58.80 | | | O |
| ANISOU | 1326 | OD2 | ASP | A | 549 | 8211 | 6535 | 7594 | −1329 | 1776 | −733 | | O |
| ATOM | 1327 | C | ASP | A | 549 | 0.379 | −5.635 | 17.218 | 1.00 | 29.10 | | | C |
| ANISOU | 1327 | C | ASP | A | 549 | 4629 | 2606 | 3823 | −644 | 1435 | −172 | | C |
| ATOM | 1328 | O | ASP | A | 549 | 0.395 | −6.281 | 18.273 | 1.00 | 27.54 | | | O |
| ANISOU | 1328 | O | ASP | A | 549 | 4194 | 2643 | 3625 | −768 | 1199 | −288 | | O |
| ATOM | 1329 | N | GLU | A | 550 | −0.576 | −4.735 | 16.952 | 1.00 | 26.01 | | | N |
| ANISOU | 1329 | N | GLU | A | 550 | 4522 | 1959 | 3399 | −399 | 1671 | −15 | | N |
| ATOM | 1330 | CA | GLU | A | 550 | −1.674 | −4.529 | 17.900 | 1.00 | 21.91 | | | C |
| ANISOU | 1330 | CA | GLU | A | 550 | 4029 | 1438 | 2859 | −291 | 1629 | 15 | | C |
| ATOM | 1331 | CB | GLU | A | 550 | −2.634 | −3.465 | 17.368 | 1.00 | 28.21 | | | C |
| ANISOU | 1331 | CB | GLU | A | 550 | 5104 | 2049 | 3566 | 77 | 1902 | 207 | | C |
| ATOM | 1332 | CG | GLU | A | 550 | −2.031 | −2.066 | 17.257 | 1.00 | 40.91 | | | C |
| ANISOU | 1332 | CG | GLU | A | 550 | 6944 | 3428 | 5171 | −12 | 2334 | 72 | | C |
| ATOM | 1333 | CD | GLU | A | 550 | −1.970 | −1.333 | 18.599 | 1.00 | 53.18 | | | C |
| ANISOU | 1333 | CD | GLU | A | 550 | 8544 | 4907 | 6753 | −274 | 2509 | −147 | | C |
| ATOM | 1334 | OE1 | GLU | A | 550 | −1.441 | −1.905 | 19.578 | 1.00 | 49.98 | | | O |
| ANISOU | 1334 | OE1 | GLU | A | 550 | 7916 | 4666 | 6408 | −604 | 2300 | −355 | | O |
| ATOM | 1335 | OE2 | GLU | A | 550 | −2.462 | −0.184 | 18.676 | 1.00 | 58.93 | | | O |
| ANISOU | 1335 | OE2 | GLU | A | 550 | 9507 | 5455 | 7427 | −139 | 2840 | −100 | | O |
| ATOM | 1336 | C | GLU | A | 550 | −2.440 | −5.818 | 18.188 | 1.00 | 25.05 | | | C |
| ANISOU | 1336 | C | GLU | A | 550 | 4133 | 2177 | 3209 | −143 | 1187 | 81 | | C |
| ATOM | 1337 | O | GLU | A | 550 | −3.043 | −5.949 | 19.264 | 1.00 | 18.95 | | | O |
| ANISOU | 1337 | O | GLU | A | 550 | 3281 | 1478 | 2441 | −172 | 1077 | 26 | | O |
| ATOM | 1338 | N | TYR | A | 551 | −2.413 | −6.776 | 17.256 | 1.00 | 24.06 | | | N |
| ANISOU | 1338 | N | TYR | A | 551 | 3860 | 2244 | 3037 | −12 | 972 | 177 | | N |
| ATOM | 1339 | CA | TYR | A | 551 | −3.130 | −8.040 | 17.372 | 1.00 | 22.10 | | | C |
| ANISOU | 1339 | CA | TYR | A | 551 | 3371 | 2282 | 2743 | 91 | 638 | 208 | | C |
| ATOM | 1340 | CB | TYR | A | 551 | −3.593 | −8.522 | 15.986 | 1.00 | 24.59 | | | C |
| ANISOU | 1340 | CB | TYR | A | 551 | 3654 | 2740 | 2948 | 336 | 562 | 353 | | C |
| ATOM | 1341 | CG | TYR | A | 551 | −4.721 | −7.725 | 15.386 | 1.00 | 35.85 | | | C |
| ANISOU | 1341 | CG | TYR | A | 551 | 5217 | 4176 | 4227 | 687 | 692 | 534 | | C |
| ATOM | 1342 | CD2 | TYR | A | 551 | −6.043 | −8.124 | 15.547 | 1.00 | 36.45 | | | C |
| ANISOU | 1342 | CD2 | TYR | A | 551 | 5152 | 4515 | 4182 | 876 | 534 | 575 | | C |
| ATOM | 1343 | CE2 | TYR | A | 551 | −7.083 | −7.390 | 14.979 | 1.00 | 46.31 | | | C |
| ANISOU | 1343 | CE2 | TYR | A | 551 | 6481 | 5875 | 5238 | 1260 | 654 | 761 | | C |
| ATOM | 1344 | CZ | TYR | A | 551 | −6.798 | −6.253 | 14.245 | 1.00 | 48.82 | | | C |
| ANISOU | 1344 | CZ | TYR | A | 551 | 7066 | 5983 | 5501 | 1481 | 967 | 927 | | C |
| ATOM | 1345 | OH | TYR | A | 551 | −7.816 | −5.515 | 13.682 | 1.00 | 52.45 | | | O |
| ANISOU | 1345 | OH | TYR | A | 551 | 7521 | 6620 | 5787 | 1827 | 1103 | 1019 | | O |
| ATOM | 1346 | CE1 | TYR | A | 551 | −5.491 | −5.844 | 14.074 | 1.00 | 46.68 | | | C |
| ANISOU | 1346 | CE1 | TYR | A | 551 | 6994 | 5369 | 5375 | 1273 | 1167 | 890 | | C |
| ATOM | 1347 | CD1 | TYR | A | 551 | −4.464 | −6.578 | 14.641 | 1.00 | 39.09 | | | C |
| ANISOU | 1347 | CD1 | TYR | A | 551 | 5898 | 4360 | 4595 | 856 | 1009 | 672 | | C |
| ATOM | 1348 | C | TYR | A | 551 | −2.315 | −9.173 | 17.982 | 1.00 | 21.94 | | | C |
| ANISOU | 1348 | C | TYR | A | 551 | 3120 | 2434 | 2783 | −131 | 431 | 73 | | C |
| ATOM | 1349 | O | TYR | A | 551 | −2.902 | −10.183 | 18.378 | 1.00 | 21.62 | | | O |
| ANISOU | 1349 | O | TYR | A | 551 | 2927 | 2563 | 2724 | −84 | 231 | 72 | | O |
| ATOM | 1350 | N | THR | A | 552 | −0.988 | −9.074 | 18.025 | 1.00 | 21.39 | | | N |
| ANISOU | 1350 | N | THR | A | 552 | 3018 | 2349 | 2761 | −347 | 503 | −35 | | N |
| ATOM | 1351 | CA | THR | A | 552 | −0.197 | −10.286 | 18.189 | 1.00 | 27.02 | | | C |
| ANISOU | 1351 | CA | THR | A | 552 | 3510 | 3280 | 3476 | −429 | 324 | −83 | | C |
| ATOM | 1352 | CB | THR | A | 552 | 0.897 | −10.353 | 17.113 | 1.00 | 27.45 | | | C |
| ANISOU | 1352 | CB | THR | A | 552 | 3569 | 3322 | 3537 | −484 | 395 | −74 | | C |
| ATOM | 1353 | OG1 | THR | A | 552 | 1.742 | −9.201 | 17.201 | 1.00 | 28.16 | | | O |
| ANISOU | 1353 | OG1 | THR | A | 552 | 3751 | 3290 | 3657 | −692 | 628 | −200 | | O |
| ATOM | 1354 | CG2 | THR | A | 552 | 0.266 | −10.424 | 15.716 | 1.00 | 30.35 | | | C |
| ANISOU | 1354 | CG2 | THR | A | 552 | 4056 | 3604 | 3870 | −272 | 408 | 81 | | C |
| ATOM | 1355 | C | THR | A | 552 | 0.431 | −10.453 | 19.571 | 1.00 | 28.62 | | | C |
| ANISOU | 1355 | C | THR | A | 552 | 3546 | 3656 | 3672 | −603 | 273 | −230 | | C |
| ATOM | 1356 | O | THR | A | 552 | 1.000 | −11.515 | 19.844 | 1.00 | 30.19 | | | O |
| ANISOU | 1356 | O | THR | A | 552 | 3564 | 4071 | 3835 | −587 | 145 | −226 | | O |
| ATOM | 1357 | N | SER | A | 553 | 0.342 | −9.463 | 20.450 | 1.00 | 26.05 | | | N |
| ANISOU | 1357 | N | SER | A | 553 | 3276 | 3267 | 3354 | −744 | 394 | −353 | | N |
| ATOM | 1358 | CA | SER | A | 553 | 0.812 | −9.646 | 21.817 | 1.00 | 33.54 | | | C |
| ANISOU | 1358 | CA | SER | A | 553 | 4028 | 4465 | 4251 | −889 | 326 | −505 | | C |
| ATOM | 1359 | CB | SER | A | 553 | 2.231 | −9.083 | 22.023 | 1.00 | 34.52 | | | C |
| ANISOU | 1359 | CB | SER | A | 553 | 4029 | 4761 | 4328 | −1180 | 460 | −728 | | C |
| ATOM | 1360 | OG | SER | A | 553 | 2.185 | −7.696 | 22.304 | 1.00 | 38.72 | | | O |
| ANISOU | 1360 | OG | SER | A | 553 | 4750 | 5087 | 4873 | −1336 | 707 | −871 | | O |
| ATOM | 1361 | C | SER | A | 553 | −0.159 | −8.972 | 22.775 | 1.00 | 27.08 | | | C |
| ANISOU | 1361 | C | SER | A | 553 | 3308 | 3536 | 3447 | −902 | 374 | −550 | | C |
| ATOM | 1362 | O | SER | A | 553 | −0.849 | −8.016 | 22.409 | 1.00 | 24.40 | | | O |
| ANISOU | 1362 | O | SER | A | 553 | 3209 | 2908 | 3156 | −868 | 549 | −510 | | O |
| ATOM | 1363 | N | SER | A | 554 | −0.194 | −9.480 | 24.012 | 1.00 | 29.48 | | | N |
| ANISOU | 1363 | N | SER | A | 554 | 3431 | 4079 | 3689 | −914 | 241 | −613 | | N |
| ATOM | 1364 | CA | SER | A | 554 | −1.102 | −8.952 | 25.027 | 1.00 | 36.01 | | | C |
| ANISOU | 1364 | CA | SER | A | 554 | 4330 | 4830 | 4524 | −925 | 266 | −659 | | C |
| ATOM | 1365 | CB | SER | A | 554 | −0.841 | −9.644 | 26.358 | 1.00 | 39.38 | | | C |
| ANISOU | 1365 | CB | SER | A | 554 | 4516 | 5598 | 4846 | −924 | 117 | −726 | | C |
| ATOM | 1366 | OG | SER | A | 554 | 0.528 | −9.509 | 26.701 | 1.00 | 39.53 | | | O |
| ANISOU | 1366 | OG | SER | A | 554 | 4397 | 5877 | 4745 | −1010 | 169 | −838 | | O |
| ATOM | 1367 | C | SER | A | 554 | −0.966 | −7.445 | 25.197 | 1.00 | 45.93 | | | C |
| ANISOU | 1367 | C | SER | A | 554 | 5765 | 5876 | 5809 | −1165 | 553 | −833 | | C |
| ATOM | 1368 | O | SER | A | 554 | −1.936 | −6.775 | 25.568 | 1.00 | 56.25 | | | O |
| ANISOU | 1368 | O | SER | A | 554 | 7257 | 6964 | 7153 | −1116 | 657 | −807 | | O |
| ATOM | 1369 | N | VAL | A | 555 | 0.224 | −6.894 | 24.947 | 1.00 | 48.06 | | | N |
| ANISOU | 1369 | N | VAL | A | 555 | 6012 | 6216 | 6033 | −1332 | 705 | −970 | | N |
| ATOM | 1370 | CA | VAL | A | 555 | 0.392 | −5.445 | 24.997 | 1.00 | 54.57 | | | C |
| ANISOU | 1370 | CA | VAL | A | 555 | 7053 | 6822 | 6861 | −1492 | 1043 | −1110 | | C |
| ATOM | 1371 | CB | VAL | A | 555 | 1.879 | −5.059 | 24.868 | 1.00 | 48.92 | | | C |
| ANISOU | 1371 | CB | VAL | A | 555 | 6236 | 6305 | 6047 | −1693 | 1177 | −1316 | | C |
| ATOM | 1372 | CG1 | VAL | A | 555 | 2.742 | −5.961 | 25.737 | 0.00 | 47.21 | | | C |
| ANISOU | 1372 | CG1 | VAL | A | 555 | 5670 | 6591 | 5676 | −1704 | 941 | −1400 | | C |
| ATOM | 1373 | CG2 | VAL | A | 555 | 2.320 | −5.105 | 23.414 | 0.00 | 46.94 | | | C |
| ANISOU | 1373 | CG2 | VAL | A | 555 | 6093 | 5880 | 5862 | −1656 | 1246 | −1229 | | C |
| ATOM | 1374 | C | VAL | A | 555 | −0.418 | −4.751 | 23.912 | 1.00 | 60.67 | | | C |
| ANISOU | 1374 | C | VAL | A | 555 | 8173 | 7139 | 7741 | −1364 | 1268 | −948 | | C |
| ATOM | 1375 | O | VAL | A | 555 | −0.673 | −3.544 | 24.009 | 1.00 | 68.61 | | | O |
| ANISOU | 1375 | O | VAL | A | 555 | 9420 | 7903 | 8746 | −1393 | 1586 | −991 | | O |
| ATOM | 1376 | N | GLY | A | 556 | −0.833 | −5.487 | 22.882 | 1.00 | 50.07 | | | N |
| ANISOU | 1376 | N | GLY | A | 556 | 6868 | 5697 | 6461 | −1183 | 1144 | −741 | | N |
| ATOM | 1377 | CA | GLY | A | 556 | −1.506 | −4.870 | 21.757 | 1.00 | 41.47 | | | C |
| ANISOU | 1377 | CA | GLY | A | 556 | 6084 | 4273 | 5399 | −935 | 1348 | −527 | | C |
| ATOM | 1378 | C | GLY | A | 556 | −2.943 | −4.503 | 22.073 | 1.00 | 32.07 | | | C |
| ANISOU | 1378 | C | GLY | A | 556 | 5054 | 2940 | 4189 | −664 | 1378 | −364 | | C |
| ATOM | 1379 | O | GLY | A | 556 | −3.633 | −5.168 | 22.844 | 1.00 | 32.35 | | | O |
| ANISOU | 1379 | O | GLY | A | 556 | 4922 | 3165 | 4204 | −584 | 1118 | −344 | | O |
| ATOM | 1380 | N | SER | A | 557 | −3.401 | −3.420 | 21.442 | 1.00 | 31.24 | | | N |
| ANISOU | 1380 | N | SER | A | 557 | 5272 | 2528 | 4068 | −479 | 1719 | −231 | | N |
| ATOM | 1381 | CA | SER | A | 557 | −4.743 | −2.909 | 21.669 | 1.00 | 29.12 | | | C |
| ANISOU | 1381 | CA | SER | A | 557 | 5165 | 2158 | 3743 | −169 | 1805 | −52 | | C |
| ATOM | 1382 | CB | SER | A | 557 | −4.872 | −1.505 | 21.078 | 1.00 | 37.95 | | | C |
| ANISOU | 1382 | CB | SER | A | 557 | 6565 | 3078 | 4776 | −1 | 2234 | 35 | | C |
| ATOM | 1383 | OG | SER | A | 557 | −4.769 | −1.538 | 19.666 | 1.00 | 41.51 | | | O |
| ANISOU | 1383 | OG | SER | A | 557 | 7079 | 3527 | 5166 | 251 | 2284 | 206 | | O |
| ATOM | 1384 | C | SER | A | 557 | −5.820 | −3.808 | 21.089 | 1.00 | 26.56 | | | C |
| ANISOU | 1384 | C | SER | A | 557 | 4702 | 2055 | 3335 | 210 | 1498 | 188 | | C |
| ATOM | 1385 | O | SER | A | 557 | −6.997 | −3.605 | 21.402 | 1.00 | 33.12 | | | O |
| ANISOU | 1385 | O | SER | A | 557 | 5575 | 2913 | 4096 | 466 | 1491 | 317 | | O |
| ATOM | 1386 | N | LYS | A | 558 | −5.448 | −4.806 | 20.285 | 1.00 | 21.09 | | | N |
| ANISOU | 1386 | N | LYS | A | 558 | 3800 | 1592 | 2622 | 232 | 1241 | 217 | | N |
| ATOM | 1387 | CA | LYS | A | 558 | −6.409 | −5.658 | 19.590 | 1.00 | 21.48 | | | C |
| ANISOU | 1387 | CA | LYS | A | 558 | 3675 | 1930 | 2555 | 534 | 979 | 378 | | C |
| ATOM | 1388 | CB | LYS | A | 558 | −6.286 | −5.472 | 18.076 | 1.00 | 21.63 | | | C |
| ANISOU | 1388 | CB | LYS | A | 558 | 3786 | 1956 | 2477 | 772 | 1087 | 546 | | C |
| ATOM | 1389 | CG | LYS | A | 558 | −6.387 | −4.031 | 17.614 | 1.00 | 27.35 | | | C |
| ANISOU | 1389 | CG | LYS | A | 558 | 4867 | 2379 | 3147 | 995 | 1523 | 699 | | C |
| ATOM | 1390 | CD | LYS | A | 558 | −7.779 | −3.483 | 17.751 | 1.00 | 31.05 | | | C |
| ANISOU | 1390 | CD | LYS | A | 558 | 5344 | 2998 | 3456 | 1334 | 1579 | 829 | | C |
| ATOM | 1391 | CE | LYS | A | 558 | −7.784 | −1.996 | 17.441 | 0.00 | 33.40 | | | C |
| ANISOU | 1391 | CE | LYS | A | 558 | 5926 | 3081 | 3684 | 1476 | 2036 | 883 | | C |
| ATOM | 1392 | NZ | LYS | A | 558 | −9.151 | −1.420 | 17.476 | 0.00 | 34.82 | | | N |
| ANISOU | 1392 | NZ | LYS | A | 558 | 6101 | 3431 | 3700 | 1811 | 2077 | 1040 | | N |
| ATOM | 1393 | C | LYS | A | 558 | −6.244 | −7.129 | 19.936 | 1.00 | 22.73 | | | C |
| ANISOU | 1393 | C | LYS | A | 558 | 3517 | 2370 | 2751 | 368 | 614 | 259 | | C |
| ATOM | 1394 | O | LYS | A | 558 | −6.814 | −7.984 | 19.249 | 1.00 | 21.51 | | | O |
| ANISOU | 1394 | O | LYS | A | 558 | 3204 | 2456 | 2512 | 514 | 429 | 320 | | O |
| ATOM | 1395 | N | PHE | A | 559 | −5.476 | −7.440 | 20.975 | 1.00 | 16.67 | | | N |
| ANISOU | 1395 | N | PHE | A | 559 | 2656 | 1597 | 2081 | 77 | 547 | 83 | | N |
| ATOM | 1396 | CA | PHE | A | 559 | −5.275 | −8.814 | 21.384 | 1.00 | 14.29 | | | C |
| ANISOU | 1396 | CA | PHE | A | 559 | 2106 | 1524 | 1799 | −20 | 279 | 10 | | C |
| ATOM | 1397 | CB | PHE | A | 559 | −4.094 | −8.891 | 22.349 | 1.00 | 15.43 | | | C |
| ANISOU | 1397 | CB | PHE | A | 559 | 2163 | 1703 | 1998 | −291 | 278 | −156 | | C |
| ATOM | 1398 | CG | PHE | A | 559 | −3.669 | −10.281 | 22.687 | 1.00 | 20.07 | | | C |
| ANISOU | 1398 | CG | PHE | A | 559 | 2534 | 2515 | 2578 | −321 | 75 | −181 | | C |
| ATOM | 1399 | CD1 | PHE | A | 559 | −2.782 | −10.969 | 21.861 | 1.00 | 18.38 | | | C |
| ANISOU | 1399 | CD1 | PHE | A | 559 | 2246 | 2377 | 2359 | −337 | 38 | −159 | | C |
| ATOM | 1400 | CE1 | PHE | A | 559 | −2.373 | −12.255 | 22.176 | 1.00 | 23.59 | | | C |
| ANISOU | 1400 | CE1 | PHE | A | 559 | 2753 | 3212 | 3000 | −315 | −79 | −149 | | C |
| ATOM | 1401 | CZ | PHE | A | 559 | −2.838 | −12.870 | 23.330 | 1.00 | 22.12 | | | C |
| ANISOU | 1401 | CZ | PHE | A | 559 | 2490 | 3121 | 2795 | −272 | −153 | −158 | | C |
| ATOM | 1402 | CE2 | PHE | A | 559 | −3.711 | −12.186 | 24.178 | 1.00 | 19.16 | | | C |
| ANISOU | 1402 | CE2 | PHE | A | 559 | 2166 | 2686 | 2430 | −279 | −146 | −197 | | C |
| ATOM | 1403 | CD2 | PHE | A | 559 | −4.120 | −10.893 | 23.850 | 1.00 | 16.36 | | | C |
| ANISOU | 1403 | CD2 | PHE | A | 559 | 1959 | 2160 | 2097 | −308 | −36 | −210 | | C |
| ATOM | 1404 | C | PHE | A | 559 | −6.545 | −9.338 | 22.055 | 1.00 | 20.09 | | | C |
| ANISOU | 1404 | C | PHE | A | 559 | 2750 | 2383 | 2501 | 94 | 137 | 32 | | C |
| ATOM | 1405 | O | PHE | A | 559 | −7.271 | −8.576 | 22.691 | 1.00 | 17.98 | | | O |
| ANISOU | 1405 | O | PHE | A | 559 | 2577 | 2031 | 2222 | 160 | 220 | 49 | | O |
| ATOM | 1406 | N | PRO | A | 560 | −6.854 | −10.674 | 21.908 | 1.00 | 18.19 | | | N |
| ANISOU | 1406 | N | PRO | A | 560 | 2338 | 2328 | 2243 | 108 | −42 | 19 | | N |
| ATOM | 1407 | CA | PRO | A | 560 | −8.097 | −11.246 | 22.501 | 1.00 | 18.96 | | | C |
| ANISOU | 1407 | CA | PRO | A | 560 | 2345 | 2548 | 2310 | 177 | −138 | 3 | | C |
| ATOM | 1408 | CB | PRO | A | 560 | −8.300 | −12.516 | 21.672 | 1.00 | 19.70 | | | C |
| ANISOU | 1408 | CB | PRO | A | 560 | 2314 | 2805 | 2367 | 171 | −220 | −26 | | C |
| ATOM | 1409 | CG | PRO | A | 560 | −6.871 | −12.938 | 21.319 | 1.00 | 18.11 | | | C |
| ANISOU | 1409 | CG | PRO | A | 560 | 2122 | 2537 | 2224 | 61 | −203 | −31 | | C |
| ATOM | 1410 | CD | PRO | A | 560 | −6.158 | −11.634 | 21.034 | 1.00 | 14.39 | | | C |
| ANISOU | 1410 | CD | PRO | A | 560 | 1775 | 1928 | 1764 | 65 | −100 | 15 | | C |
| ATOM | 1411 | C | PRO | A | 560 | −7.951 | −11.539 | 23.991 | 1.00 | 17.20 | | | C |
| ANISOU | 1411 | C | PRO | A | 560 | 2085 | 2301 | 2150 | 64 | −177 | −73 | | C |
| ATOM | 1412 | O | PRO | A | 560 | −7.996 | −12.691 | 24.465 | 1.00 | 13.23 | | | O |
| ANISOU | 1412 | O | PRO | A | 560 | 1485 | 1878 | 1664 | 23 | −246 | −112 | | O |
| ATOM | 1413 | N | VAL | A | 561 | −7.776 | −10.462 | 24.753 | 1.00 | 10.56 | | | N |
| ANISOU | 1413 | N | VAL | A | 561 | 1340 | 1343 | 1330 | 19 | −90 | −95 | | N |
| ATOM | 1414 | CA | VAL | A | 561 | −7.584 | −10.569 | 26.199 | 1.00 | 10.33 | | | C |
| ANISOU | 1414 | CA | VAL | A | 561 | 1261 | 1338 | 1325 | −85 | −120 | −181 | | C |
| ATOM | 1415 | CB | VAL | A | 561 | −7.504 | −9.168 | 26.822 | 1.00 | 11.63 | | | C |
| ANISOU | 1415 | CB | VAL | A | 561 | 1563 | 1356 | 1499 | −165 | 34 | −239 | | C |
| ATOM | 1416 | CG1 | VAL | A | 561 | −7.568 | −9.273 | 28.348 | 1.00 | 14.23 | | | C |
| ANISOU | 1416 | CG1 | VAL | A | 561 | 1821 | 1765 | 1823 | −252 | −12 | −337 | | C |
| ATOM | 1417 | CG2 | VAL | A | 561 | −6.225 | −8.430 | 26.371 | 1.00 | 16.65 | | | C |
| ANISOU | 1417 | CG2 | VAL | A | 561 | 2266 | 1912 | 2147 | −336 | 185 | −319 | | C |
| ATOM | 1418 | C | VAL | A | 561 | −8.705 | −11.382 | 26.838 | 1.00 | 11.40 | | | C |
| ANISOU | 1418 | C | VAL | A | 561 | 1325 | 1553 | 1454 | −2 | −213 | −169 | | C |
| ATOM | 1419 | O | VAL | A | 561 | −8.468 | −12.249 | 27.683 | 1.00 | 9.04 | | | O |
| ANISOU | 1419 | O | VAL | A | 561 | 942 | 1331 | 1160 | −35 | −264 | −201 | | O |
| ATOM | 1420 | N | ARG | A | 562 | −9.945 | −11.098 | 26.447 | 1.00 | 9.80 | | | N |
| ANISOU | 1420 | N | ARG | A | 562 | 1150 | 1358 | 1216 | 128 | −207 | −120 | | N |
| ATOM | 1421 | CA | ARG | A | 562 | −11.115 | −11.677 | 27.096 | 1.00 | 11.24 | | | C |
| ANISOU | 1421 | CA | ARG | A | 562 | 1260 | 1626 | 1385 | 176 | −263 | −143 | | C |
| ATOM | 1422 | CB | ARG | A | 562 | −12.371 | −10.928 | 26.655 | 1.00 | 14.98 | | | C |
| ANISOU | 1422 | CB | ARG | A | 562 | 1752 | 2166 | 1775 | 349 | −234 | −82 | | C |
| ATOM | 1423 | CG | ARG | A | 562 | −12.369 | −9.448 | 27.011 | 1.00 | 11.47 | | | C |
| ANISOU | 1423 | CG | ARG | A | 562 | 1484 | 1548 | 1326 | 431 | −100 | −12 | | C |
| ATOM | 1424 | CD | ARG | A | 562 | −13.439 | −8.654 | 26.255 | 1.00 | 13.05 | | | C |
| ANISOU | 1424 | CD | ARG | A | 562 | 1728 | 1836 | 1396 | 710 | −18 | 120 | | C |
| ATOM | 1425 | NE | ARG | A | 562 | −13.151 | −7.233 | 26.363 | 1.00 | 14.53 | | | N |
| ANISOU | 1425 | NE | ARG | A | 562 | 2168 | 1762 | 1589 | 794 | 212 | 210 | | N |
| ATOM | 1426 | CZ | ARG | A | 562 | −13.662 | −6.421 | 27.286 | 1.00 | 15.35 | | | C |
| ANISOU | 1426 | CZ | ARG | A | 562 | 2406 | 1722 | 1702 | 847 | 340 | 231 | | C |
| ATOM | 1427 | NH1 | ARG | A | 562 | −14.544 | −6.878 | 28.169 | 1.00 | 14.65 | | | N |
| ANISOU | 1427 | NH1 | ARG | A | 562 | 2196 | 1759 | 1610 | 853 | 219 | 188 | | N |
| ATOM | 1428 | NH2 | ARG | A | 562 | −13.308 | −5.138 | 27.301 | 1.00 | 17.15 | | | N |
| ANISOU | 1428 | NH2 | ARG | A | 562 | 2915 | 1658 | 1943 | 888 | 633 | 290 | | N |
| ATOM | 1429 | C | ARG | A | 562 | −11.287 | −13.156 | 26.796 | 1.00 | 13.23 | | | C |
| ANISOU | 1429 | C | ARG | A | 562 | 1402 | 1985 | 1640 | 127 | −305 | −195 | | C |
| ATOM | 1430 | O | ARG | A | 562 | −12.170 | −13.792 | 27.385 | 1.00 | 8.99 | | | O |
| ANISOU | 1430 | O | ARG | A | 562 | 814 | 1499 | 1104 | 118 | −305 | −249 | | O |
| ATOM | 1431 | N | TRP | A | 563 | −10.513 | −13.705 | 25.862 | 1.00 | 11.14 | | | N |
| ANISOU | 1431 | N | TRP | A | 563 | 1121 | 1735 | 1377 | 85 | −298 | −192 | | N |
| ATOM | 1432 | CA | TRP | A | 563 | −10.527 | −15.132 | 25.575 | 1.00 | 8.70 | | | C |
| ANISOU | 1432 | CA | TRP | A | 563 | 761 | 1466 | 1079 | 15 | −260 | −253 | | C |
| ATOM | 1433 | CB | TRP | A | 563 | −10.743 | −15.363 | 24.076 | 1.00 | 9.26 | | | C |
| ANISOU | 1433 | CB | TRP | A | 563 | 776 | 1657 | 1085 | 2 | −257 | −289 | | C |
| ATOM | 1434 | CG | TRP | A | 563 | −12.153 | −15.014 | 23.628 | 1.00 | 10.51 | | | C |
| ANISOU | 1434 | CG | TRP | A | 563 | 827 | 2036 | 1130 | 60 | −289 | −346 | | C |
| ATOM | 1435 | CD1 | TRP | A | 563 | −13.190 | −15.877 | 23.466 | 1.00 | 11.13 | | | C |
| ANISOU | 1435 | CD1 | TRP | A | 563 | 790 | 2288 | 1149 | −37 | −243 | −500 | | C |
| ATOM | 1436 | NE1 | TRP | A | 563 | −14.323 | −15.192 | 23.068 | 1.00 | 12.35 | | | N |
| ANISOU | 1436 | NE1 | TRP | A | 563 | 869 | 2653 | 1172 | 84 | −259 | −487 | | N |
| ATOM | 1437 | CE2 | TRP | A | 563 | −14.028 | −13.856 | 22.977 | 1.00 | 12.28 | | | C |
| ANISOU | 1437 | CE2 | TRP | A | 563 | 922 | 2616 | 1129 | 288 | −321 | −321 | | C |
| ATOM | 1438 | CD2 | TRP | A | 563 | −12.668 | −13.702 | 23.328 | 1.00 | 12.37 | | | C |
| ANISOU | 1438 | CD2 | TRP | A | 563 | 1063 | 2365 | 1270 | 247 | −330 | −248 | | C |
| ATOM | 1439 | CE3 | TRP | A | 563 | −12.109 | −12.418 | 23.310 | 1.00 | 15.09 | | | C |
| ANISOU | 1439 | CE3 | TRP | A | 563 | 1548 | 2558 | 1626 | 376 | −302 | −105 | | C |
| ATOM | 1440 | CZ3 | TRP | A | 563 | −12.927 | −11.332 | 22.926 | 1.00 | 12.83 | | | C |
| ANISOU | 1440 | CZ3 | TRP | A | 563 | 1282 | 2383 | 1209 | 614 | −263 | 2 | | C |
| ATOM | 1441 | CH2 | TRP | A | 563 | −14.282 | −11.523 | 22.594 | 1.00 | 16.25 | | | C |
| ANISOU | 1441 | CH2 | TRP | A | 563 | 1548 | 3145 | 1483 | 723 | −293 | −38 | | C |
| ATOM | 1442 | CZ2 | TRP | A | 563 | −14.845 | −12.771 | 22.600 | 1.00 | 13.71 | | | C |
| ANISOU | 1442 | CZ2 | TRP | A | 563 | 1087 | 2956 | 1166 | 516 | −308 | −220 | | C |
| ATOM | 1443 | C | TRP | A | 563 | −9.243 | −15.817 | 26.054 | 1.00 | 8.37 | | | C |
| ANISOU | 1443 | C | TRP | A | 563 | 754 | 1343 | 1082 | −7 | −214 | −211 | | C |
| ATOM | 1444 | O | TRP | A | 563 | −8.986 | −16.965 | 25.684 | 1.00 | 12.04 | | | O |
| ANISOU | 1444 | O | TRP | A | 563 | 1232 | 1785 | 1556 | −35 | −121 | −226 | | O |
| ATOM | 1445 | N | SER | A | 564 | −8.446 | −15.150 | 26.900 | 1.00 | 10.87 | | | N |
| ANISOU | 1445 | N | SER | A | 564 | 1080 | 1650 | 1401 | 12 | −250 | −171 | | N |
| ATOM | 1446 | CA | SER | A | 564 | −7.118 | −15.696 | 27.167 | 1.00 | 8.49 | | | C |
| ANISOU | 1446 | CA | SER | A | 564 | 751 | 1397 | 1078 | 34 | −220 | −125 | | C |
| ATOM | 1447 | CB | SER | A | 564 | −6.039 | −14.663 | 26.829 | 1.00 | 12.78 | | | C |
| ANISOU | 1447 | CB | SER | A | 564 | 1266 | 1988 | 1601 | −32 | −260 | −137 | | C |
| ATOM | 1448 | OG | SER | A | 564 | −6.080 | −14.330 | 25.456 | 1.00 | 15.56 | | | O |
| ANISOU | 1448 | OG | SER | A | 564 | 1664 | 2271 | 1978 | −61 | −254 | −129 | | O |
| ATOM | 1449 | C | SER | A | 564 | −6.944 | −16.127 | 28.622 | 1.00 | 8.88 | | | C |
| ANISOU | 1449 | C | SER | A | 564 | 771 | 1517 | 1083 | 117 | −194 | −97 | | C |
| ATOM | 1450 | O | SER | A | 564 | −7.381 | −15.424 | 29.539 | 1.00 | 12.67 | | | O |
| ANISOU | 1450 | O | SER | A | 564 | 1240 | 2020 | 1555 | 102 | −241 | −136 | | O |
| ATOM | 1451 | N | PRO | A | 565 | −6.270 | −17.250 | 28.859 | 1.00 | 9.48 | | | N |
| ANISOU | 1451 | N | PRO | A | 565 | 847 | 1644 | 1113 | 237 | −95 | −13 | | N |
| ATOM | 1452 | CA | PRO | A | 565 | −6.026 | −17.708 | 30.227 | 1.00 | 10.35 | | | C |
| ANISOU | 1452 | CA | PRO | A | 565 | 926 | 1874 | 1131 | 397 | −44 | 55 | | C |
| ATOM | 1453 | CB | PRO | A | 565 | −5.578 | −19.166 | 30.032 | 1.00 | 11.30 | | | C |
| ANISOU | 1453 | CB | PRO | A | 565 | 1131 | 1945 | 1217 | 577 | 163 | 187 | | C |
| ATOM | 1454 | CG | PRO | A | 565 | −4.895 | −19.146 | 28.686 | 1.00 | 11.01 | | | C |
| ANISOU | 1454 | CG | PRO | A | 565 | 1084 | 1890 | 1211 | 495 | 144 | 179 | | C |
| ATOM | 1455 | CD | PRO | A | 565 | −5.748 | −18.194 | 27.855 | 1.00 | 9.74 | | | C |
| ANISOU | 1455 | CD | PRO | A | 565 | 927 | 1619 | 1155 | 270 | 13 | 41 | | C |
| ATOM | 1456 | C | PRO | A | 565 | −4.930 | −16.886 | 30.887 | 1.00 | 12.19 | | | C |
| ANISOU | 1456 | C | PRO | A | 565 | 992 | 2400 | 1239 | 402 | −152 | 29 | | C |
| ATOM | 1457 | O | PRO | A | 565 | −4.157 | −16.194 | 30.202 | 1.00 | 13.38 | | | O |
| ANISOU | 1457 | O | PRO | A | 565 | 1072 | 2629 | 1384 | 278 | −217 | −34 | | O |
| ATOM | 1458 | N | PRO | A | 566 | −4.814 | −16.957 | 32.216 | 1.00 | 8.25 | | | N |
| ANISOU | 1458 | N | PRO | A | 566 | 1064 | 981 | 1091 | −21 | −297 | −75 | | N |
| ATOM | 1459 | CA | PRO | A | 566 | −3.789 | −16.151 | 32.905 | 1.00 | 11.73 | | | C |
| ANISOU | 1459 | CA | PRO | A | 566 | 1454 | 1466 | 1538 | −47 | −338 | −126 | | C |
| ATOM | 1460 | CB | PRO | A | 566 | −3.955 | −16.538 | 34.388 | 1.00 | 14.59 | | | C |
| ANISOU | 1460 | CB | PRO | A | 566 | 1823 | 1897 | 1821 | −19 | −384 | −131 | | C |
| ATOM | 1461 | CG | PRO | A | 566 | −4.864 | −17.723 | 34.406 | 1.00 | 16.09 | | | C |
| ANISOU | 1461 | CG | PRO | A | 566 | 2063 | 2073 | 1978 | 26 | −363 | −62 | | C |
| ATOM | 1462 | CD | PRO | A | 566 | −5.692 | −17.669 | 33.155 | 1.00 | 11.53 | | | C |
| ANISOU | 1462 | CD | PRO | A | 566 | 1519 | 1416 | 1445 | 13 | −307 | −49 | | C |
| ATOM | 1463 | C | PRO | A | 566 | −2.372 | −16.401 | 32.413 | 1.00 | 14.02 | | | C |
| ANISOU | 1463 | C | PRO | A | 566 | 1674 | 1780 | 1872 | −48 | −348 | −123 | | C |
| ATOM | 1464 | O | PRO | A | 566 | −1.603 | −15.439 | 32.322 | 1.00 | 9.29 | | | O |
| ANISOU | 1464 | O | PRO | A | 566 | 1029 | 1186 | 1316 | −93 | −356 | −168 | | O |
| ATOM | 1465 | N | GLU | A | 567 | −1.994 | −17.645 | 32.086 | 1.00 | 11.41 | | | N |
| ANISOU | 1465 | N | GLU | A | 567 | 1330 | 1459 | 1545 | 0 | −343 | −77 | | N |
| ATOM | 1466 | CA | GLU | A | 567 | −0.615 | −17.867 | 31.646 | 1.00 | 9.94 | | | C |
| ANISOU | 1466 | CA | GLU | A | 567 | 1067 | 1302 | 1409 | 5 | −348 | −82 | | C |
| ATOM | 1467 | CB | GLU | A | 567 | −0.268 | −19.368 | 31.648 | 1.00 | 9.76 | | | C |
| ANISOU | 1467 | CB | GLU | A | 567 | 1027 | 1288 | 1394 | 75 | −353 | −35 | | C |
| ATOM | 1468 | CG | GLU | A | 567 | −0.976 | −20.220 | 30.578 | 1.00 | 10.35 | | | C |
| ANISOU | 1468 | CG | GLU | A | 567 | 1145 | 1300 | 1488 | 96 | −293 | −10 | | C |
| ATOM | 1469 | CD | GLU | A | 567 | −2.405 | −20.663 | 30.950 | 1.00 | 12.14 | | | C |
| ANISOU | 1469 | CD | GLU | A | 567 | 1452 | 1489 | 1673 | 109 | −286 | 21 | | C |
| ATOM | 1470 | OE1 | GLU | A | 567 | −2.943 | −20.223 | 31.995 | 1.00 | 10.39 | | | O |
| ANISOU | 1470 | OE1 | GLU | A | 567 | 1259 | 1291 | 1399 | 101 | −318 | 24 | | O |
| ATOM | 1471 | OE2 | GLU | A | 567 | −2.995 | −21.458 | 30.171 | 1.00 | 9.56 | | | O |
| ANISOU | 1471 | OE2 | GLU | A | 567 | 1154 | 1113 | 1364 | 125 | −244 | 33 | | O |
| ATOM | 1472 | C | GLU | A | 567 | −0.347 | −17.244 | 30.272 | 1.00 | 10.37 | | | C |
| ANISOU | 1472 | C | GLU | A | 567 | 1104 | 1321 | 1516 | −41 | −292 | −94 | | C |
| ATOM | 1473 | O | GLU | A | 567 | 0.799 | −16.898 | 29.973 | 1.00 | 11.27 | | | O |
| ANISOU | 1473 | O | GLU | A | 567 | 1145 | 1461 | 1674 | −64 | −292 | −115 | | O |
| ATOM | 1474 | N | VAL | A | 568 | −1.376 | −17.056 | 29.438 | 1.00 | 8.57 | | | N |
| ANISOU | 1474 | N | VAL | A | 568 | 935 | 1042 | 1281 | −57 | −245 | −77 | | N |
| ATOM | 1475 | CA | VAL | A | 568 | −1.175 | −16.285 | 28.210 | 1.00 | 9.76 | | | C |
| ANISOU | 1475 | CA | VAL | A | 568 | 1070 | 1173 | 1467 | −106 | −197 | −75 | | C |
| ATOM | 1476 | CB | VAL | A | 568 | −2.352 | −16.466 | 27.231 | 1.00 | 11.98 | | | C |
| ANISOU | 1476 | CB | VAL | A | 568 | 1412 | 1417 | 1720 | −105 | −153 | −43 | | C |
| ATOM | 1477 | CG1 | VAL | A | 568 | −2.357 | −15.351 | 26.185 | 1.00 | 11.74 | | | C |
| ANISOU | 1477 | CG1 | VAL | A | 568 | 1377 | 1371 | 1714 | −162 | −114 | −22 | | C |
| ATOM | 1478 | CG2 | VAL | A | 568 | −2.272 | −17.805 | 26.517 | 1.00 | 10.40 | | | C |
| ANISOU | 1478 | CG2 | VAL | A | 568 | 1211 | 1234 | 1509 | −64 | −124 | −36 | | C |
| ATOM | 1479 | C | VAL | A | 568 | −0.964 | −14.816 | 28.542 | 1.00 | 9.60 | | | C |
| ANISOU | 1479 | C | VAL | A | 568 | 1032 | 1134 | 1482 | −166 | −210 | −101 | | C |
| ATOM | 1480 | O | VAL | A | 568 | −0.039 | −14.173 | 28.037 | 1.00 | 12.84 | | | O |
| ANISOU | 1480 | O | VAL | A | 568 | 1385 | 1550 | 1942 | −210 | −192 | −107 | | O |
| ATOM | 1481 | N | LEU | A | 569 | −1.836 | −14.259 | 29.384 | 1.00 | 13.76 | | | N |
| ANISOU | 1481 | N | LEU | A | 569 | 1604 | 1634 | 1991 | −171 | −235 | −121 | | N |
| ATOM | 1482 | CA | LEU | A | 569 | −1.753 | −12.845 | 29.715 | 1.00 | 9.21 | | | C |
| ANISOU | 1482 | CA | LEU | A | 569 | 1014 | 1021 | 1465 | −227 | −243 | −160 | | C |
| ATOM | 1483 | CB | LEU | A | 569 | −2.955 | −12.444 | 30.559 | 1.00 | 10.18 | | | C |
| ANISOU | 1483 | CB | LEU | A | 569 | 1193 | 1113 | 1560 | −217 | −260 | −188 | | C |
| ATOM | 1484 | CG | LEU | A | 569 | −4.297 | −12.556 | 29.858 | 1.00 | 11.88 | | | C |
| ANISOU | 1484 | CG | LEU | A | 569 | 1473 | 1282 | 1761 | −198 | −223 | −137 | | C |
| ATOM | 1485 | CD1 | LEU | A | 569 | −5.404 | −12.041 | 30.787 | 1.00 | 14.53 | | | C |
| ANISOU | 1485 | CD1 | LEU | A | 569 | 1849 | 1590 | 2083 | −190 | −235 | −176 | | C |
| ATOM | 1486 | CD2 | LEU | A | 569 | −4.273 | −11.789 | 28.545 | 1.00 | 16.25 | | | C |
| ANISOU | 1486 | CD2 | LEU | A | 569 | 2019 | 1784 | 2372 | −237 | −180 | −90 | | C |
| ATOM | 1487 | C | LEU | A | 569 | −0.470 | −12.505 | 30.456 | 1.00 | 12.11 | | | C |
| ANISOU | 1487 | C | LEU | A | 569 | 1306 | 1434 | 1863 | −254 | −285 | −218 | | C |
| ATOM | 1488 | O | LEU | A | 569 | 0.072 | −11.411 | 30.271 | 1.00 | 10.59 | | | O |
| ANISOU | 1488 | O | LEU | A | 569 | 1072 | 1208 | 1742 | −316 | −276 | −245 | | O |
| ATOM | 1489 | N | MET | A | 570 | 0.027 | −13.413 | 31.292 | 1.00 | 10.29 | | | N |
| ANISOU | 1489 | N | MET | A | 570 | 1050 | 1276 | 1583 | −211 | −332 | −231 | | N |
| ATOM | 1490 | CA | MET | A | 570 | 1.176 | −13.102 | 32.129 | 1.00 | 12.15 | | | C |
| ANISOU | 1490 | CA | MET | A | 570 | 1209 | 1574 | 1835 | −233 | −386 | −289 | | C |
| ATOM | 1491 | CB | MET | A | 570 | 1.092 | −13.871 | 33.453 | 1.00 | 19.13 | | | C |
| ANISOU | 1491 | CB | MET | A | 570 | 2099 | 2538 | 2633 | −181 | −451 | −301 | | C |
| ATOM | 1492 | CG | MET | A | 570 | −0.053 | −13.441 | 34.369 | 1.00 | 17.80 | | | C |
| ANISOU | 1492 | CG | MET | A | 570 | 1996 | 2362 | 2405 | −182 | −465 | −338 | | C |
| ATOM | 1493 | SD | MET | A | 570 | −0.428 | −14.718 | 35.608 | 1.00 | 21.25 | | | S |
| ANISOU | 1493 | SD | MET | A | 570 | 2461 | 2894 | 2719 | −107 | −516 | −298 | | S |
| ATOM | 1494 | CE | MET | A | 570 | 1.138 | −14.778 | 36.487 | 1.00 | 19.47 | | | C |
| ANISOU | 1494 | CE | MET | A | 570 | 2131 | 2789 | 2479 | −109 | −602 | −336 | | C |
| ATOM | 1495 | C | MET | A | 570 | 2.509 | −13.408 | 31.464 | 1.00 | 15.42 | | | C |
| ANISOU | 1495 | C | MET | A | 570 | 1536 | 2022 | 2301 | −241 | −376 | −275 | | C |
| ATOM | 1496 | O | MET | A | 570 | 3.478 | −12.657 | 31.650 | 1.00 | 14.51 | | | O |
| ANISOU | 1496 | O | MET | A | 570 | 1345 | 1926 | 2241 | −294 | −396 | −324 | | O |
| ATOM | 1497 | N | ATYR | A | 571 | 2.565 | −14.492 | 30.683 | 0.60 | 13.08 | | | N |
| ANISOU | 1497 | N | ATYR | A | 571 | 1244 | 1732 | 1993 | −191 | −342 | −217 | | N |
| ATOM | 1498 | CA | ATYR | A | 571 | 3.823 | −15.056 | 30.206 | 0.60 | 13.17 | | | C |
| ANISOU | 1498 | CA | ATYR | A | 571 | 1168 | 1791 | 2045 | −176 | −334 | −209 | | C |
| ATOM | 1499 | CB | ATYR | A | 571 | 4.167 | −16.311 | 31.020 | 0.60 | 12.51 | | | C |
| ANISOU | 1499 | CB | ATYR | A | 571 | 1060 | 1767 | 1925 | −95 | −388 | −193 | | C |
| ATOM | 1500 | CG | ATYR | A | 571 | 4.133 | −16.145 | 32.526 | 0.60 | 16.36 | | | C |
| ANISOU | 1500 | CG | ATYR | A | 571 | 1546 | 2311 | 2361 | −87 | −473 | −220 | | C |
| ATOM | 1501 | CD1 | ATYR | A | 571 | 4.800 | −15.099 | 33.157 | 0.60 | 18.68 | | | C |
| ANISOU | 1501 | CD1 | ATYR | A | 571 | 1778 | 2645 | 2673 | −147 | −519 | −290 | | C |
| ATOM | 1502 | CE1 | ATYR | A | 571 | 4.781 | −14.968 | 34.534 | 0.60 | 27.00 | | | C |
| ANISOU | 1502 | CE1 | ATYR | A | 571 | 2827 | 3771 | 3662 | −141 | −598 | −328 | | C |
| ATOM | 1503 | CZ | ATYR | A | 571 | 4.083 | −15.891 | 35.291 | 0.60 | 31.40 | | | C |
| ANISOU | 1503 | CZ | ATYR | A | 571 | 3441 | 4361 | 4130 | −73 | −629 | −278 | | C |
| ATOM | 1504 | OH | ATYR | A | 571 | 4.043 | −15.789 | 36.657 | 0.60 | 33.73 | | | O |
| ANISOU | 1504 | OH | ATYR | A | 571 | 3730 | 4748 | 4338 | −66 | −705 | −308 | | O |
| ATOM | 1505 | CE2 | ATYR | A | 571 | 3.420 | −16.934 | 34.686 | 0.60 | 22.93 | | | C |
| ANISOU | 1505 | CE2 | ATYR | A | 571 | 2427 | 3234 | 3052 | −14 | −581 | −198 | | C |
| ATOM | 1506 | CD2 | ATYR | A | 571 | 3.452 | −17.057 | 33.316 | 0.60 | 18.76 | | | C |
| ANISOU | 1506 | CD2 | ATYR | A | 571 | 1902 | 2633 | 2592 | −22 | −507 | −179 | | C |
| ATOM | 1507 | C | ATYR | A | 571 | 3.824 | −15.425 | 28.723 | 0.60 | 13.42 | | | C |
| ANISOU | 1507 | C | ATYR | A | 571 | 1203 | 1797 | 2098 | −174 | −252 | −169 | | C |
| ATOM | 1508 | O | ATYR | A | 571 | 4.866 | −15.851 | 28.210 | 0.60 | 13.42 | | | O |
| ANISOU | 1508 | O | ATYR | A | 571 | 1126 | 1838 | 2137 | −164 | −230 | −171 | | O |
| ATOM | 1509 | N | BTYR | A | 571 | 2.618 | −14.513 | 30.715 | 0.40 | 13.52 | | | N |
| ANISOU | 1509 | N | BTYR | A | 571 | 1296 | 1791 | 2049 | −190 | −344 | −218 | | N |
| ATOM | 1510 | CA | BTYR | A | 571 | 3.907 | −14.818 | 30.096 | 0.40 | 13.06 | | | C |
| ANISOU | 1510 | CA | BTYR | A | 571 | 1147 | 1773 | 2044 | −192 | −328 | −214 | | C |
| ATOM | 1511 | CB | BTYR | A | 571 | 4.805 | −15.597 | 31.061 | 0.40 | 13.55 | | | C |
| ANISOU | 1511 | CB | BTYR | A | 571 | 1139 | 1916 | 2093 | −141 | −397 | −227 | | C |
| ATOM | 1512 | CG | BTYR | A | 571 | 4.209 | −16.848 | 31.675 | 0.40 | 14.68 | | | C |
| ANISOU | 1512 | CG | BTYR | A | 571 | 1331 | 2073 | 2172 | −55 | −428 | −183 | | C |
| ATOM | 1513 | CD1 | BTYR | A | 571 | 3.441 | −16.787 | 32.831 | 0.40 | 18.18 | | | C |
| ANISOU | 1513 | CD1 | BTYR | A | 571 | 1830 | 2533 | 2543 | −43 | −479 | −186 | | C |
| ATOM | 1514 | CE1 | BTYR | A | 571 | 2.915 | −17.941 | 33.403 | 0.40 | 19.85 | | | C |
| ANISOU | 1514 | CE1 | BTYR | A | 571 | 2083 | 2758 | 2701 | 30 | −502 | −130 | | C |
| ATOM | 1515 | CZ | BTYR | A | 571 | 3.175 | −19.170 | 32.825 | 0.40 | 16.70 | | | C |
| ANISOU | 1515 | CZ | BTYR | A | 571 | 1670 | 2339 | 2338 | 94 | −476 | −77 | | C |
| ATOM | 1516 | OH | BTYR | A | 571 | 2.667 | −20.334 | 33.372 | 0.40 | 17.77 | | | O |
| ANISOU | 1516 | OH | BTYR | A | 571 | 1844 | 2469 | 2440 | 164 | −495 | −12 | | O |
| ATOM | 1517 | CE2 | BTYR | A | 571 | 3.943 | −19.247 | 31.691 | 0.40 | 13.63 | | | C |
| ANISOU | 1517 | CE2 | BTYR | A | 571 | 1225 | 1932 | 2021 | 87 | −427 | −90 | | C |
| ATOM | 1518 | CD2 | BTYR | A | 571 | 4.462 | −18.094 | 31.129 | 0.40 | 15.09 | | | C |
| ANISOU | 1518 | CD2 | BTYR | A | 571 | 1368 | 2121 | 2243 | 12 | −402 | −139 | | C |
| ATOM | 1519 | C | BTYR | A | 571 | 3.761 | −15.565 | 28.774 | 0.40 | 13.04 | | | C |
| ANISOU | 1519 | C | BTYR | A | 571 | 1160 | 1749 | 2044 | −165 | −255 | −167 | | C |
| ATOM | 1520 | O | BTYR | A | 571 | 4.618 | −16.387 | 28.429 | 0.40 | 12.99 | | | O |
| ANISOU | 1520 | O | BTYR | A | 571 | 1091 | 1783 | 2061 | −128 | −243 | −162 | | O |
| ATOM | 1521 | N | SER | A | 572 | 2.696 | −15.280 | 28.024 | 1.00 | 10.64 | | | N |
| ANISOU | 1521 | N | SER | A | 572 | 933 | 1390 | 1719 | −183 | −207 | −138 | | N |
| ATOM | 1522 | CA | SER | A | 572 | 2.543 | −15.733 | 26.635 | 1.00 | 13.96 | | | C |
| ANISOU | 1522 | CA | SER | A | 572 | 1366 | 1805 | 2132 | −177 | −135 | −105 | | C |
| ATOM | 1523 | CB | SER | A | 572 | 3.480 | −14.979 | 25.698 | 1.00 | 19.24 | | | C |
| ANISOU | 1523 | CB | SER | A | 572 | 1966 | 2494 | 2850 | −239 | −83 | −100 | | C |
| ATOM | 1524 | OG | SER | A | 572 | 3.388 | −13.586 | 25.909 | 1.00 | 21.59 | | | O |
| ANISOU | 1524 | OG | SER | A | 572 | 2266 | 2750 | 3187 | −310 | −90 | −98 | | O |
| ATOM | 1525 | C | SER | A | 572 | 2.753 | −17.235 | 26.478 | 1.00 | 16.11 | | | C |
| ANISOU | 1525 | C | SER | A | 572 | 1627 | 2103 | 2390 | −102 | −128 | −105 | | C |
| ATOM | 1526 | O | SER | A | 572 | 3.362 | −17.692 | 25.505 | 1.00 | 14.73 | | | O |
| ANISOU | 1526 | O | SER | A | 572 | 1407 | 1955 | 2233 | −95 | −75 | −110 | | O |
| ATOM | 1527 | N | LYS | A | 573 | 2.221 | −18.022 | 27.414 | 1.00 | 11.75 | | | N |
| ANISOU | 1527 | N | LYS | A | 573 | 1114 | 1540 | 1812 | −46 | −175 | −100 | | N |
| ATOM | 1528 | CA | LYS | A | 573 | 2.334 | −19.476 | 27.310 | 1.00 | 11.96 | | | C |
| ANISOU | 1528 | CA | LYS | A | 573 | 1133 | 1566 | 1845 | 28 | −168 | −93 | | C |
| ATOM | 1529 | CB | LYS | A | 573 | 2.514 | −20.123 | 28.677 | 1.00 | 13.68 | | | C |
| ANISOU | 1529 | CB | LYS | A | 573 | 1340 | 1798 | 2062 | 84 | −240 | −75 | | C |
| ATOM | 1530 | CG | LYS | A | 573 | 2.827 | −21.621 | 28.539 | 1.00 | 17.08 | | | C |
| ANISOU | 1530 | CG | LYS | A | 573 | 1746 | 2213 | 2532 | 165 | −230 | −59 | | C |
| ATOM | 1531 | CD | LYS | A | 573 | 3.214 | −22.293 | 29.834 | 1.00 | 20.86 | | | C |
| ANISOU | 1531 | CD | LYS | A | 573 | 2196 | 2713 | 3016 | 227 | −303 | −19 | | C |
| ATOM | 1532 | CE | LYS | A | 573 | 3.774 | −23.688 | 29.565 | 1.00 | 21.96 | | | C |
| ANISOU | 1532 | CE | LYS | A | 573 | 2290 | 2825 | 3230 | 309 | −287 | −3 | | C |
| ATOM | 1533 | NZ | LYS | A | 573 | 2.792 | −24.584 | 28.894 | 1.00 | 22.28 | | | N |
| ANISOU | 1533 | NZ | LYS | A | 573 | 2400 | 2783 | 3284 | 330 | −232 | −1 | | N |
| ATOM | 1534 | C | LYS | A | 573 | 1.096 | −20.028 | 26.604 | 1.00 | 12.69 | | | C |
| ANISOU | 1534 | C | LYS | A | 573 | 1307 | 1612 | 1903 | 39 | −127 | −81 | | C |
| ATOM | 1535 | O | LYS | A | 573 | 0.060 | −20.242 | 27.229 | 1.00 | 12.80 | | | O |
| ANISOU | 1535 | O | LYS | A | 573 | 1388 | 1593 | 1884 | 53 | −153 | −61 | | O |
| ATOM | 1536 | N | PHE | A | 574 | 1.217 | −20.295 | 25.303 | 1.00 | 14.48 | | | N |
| ANISOU | 1536 | N | PHE | A | 574 | 1521 | 1846 | 2133 | 30 | −61 | −99 | | N |
| ATOM | 1537 | CA | PHE | A | 574 | 0.150 | −20.920 | 24.526 | 1.00 | 11.53 | | | C |
| ANISOU | 1537 | CA | PHE | A | 574 | 1210 | 1444 | 1725 | 38 | −24 | −104 | | C |
| ATOM | 1538 | CB | PHE | A | 574 | 0.196 | −20.409 | 23.083 | 1.00 | 11.89 | | | C |
| ANISOU | 1538 | CB | PHE | A | 574 | 1248 | 1531 | 1740 | −11 | 41 | −115 | | C |
| ATOM | 1539 | CG | PHE | A | 574 | −0.148 | −18.962 | 22.962 | 1.00 | 14.40 | | | C |
| ANISOU | 1539 | CG | PHE | A | 574 | 1587 | 1853 | 2031 | −76 | 36 | −75 | | C |
| ATOM | 1540 | CD1 | PHE | A | 574 | 0.824 | −17.988 | 23.148 | 1.00 | 17.53 | | | C |
| ANISOU | 1540 | CD1 | PHE | A | 574 | 1926 | 2270 | 2463 | −116 | 34 | −65 | | C |
| ATOM | 1541 | CE1 | PHE | A | 574 | 0.504 | −16.641 | 23.051 | 1.00 | 17.42 | | | C |
| ANISOU | 1541 | CE1 | PHE | A | 574 | 1931 | 2239 | 2449 | −176 | 33 | −27 | | C |
| ATOM | 1542 | CZ | PHE | A | 574 | −0.814 | −16.265 | 22.785 | 1.00 | 11.35 | | | C |
| ANISOU | 1542 | CZ | PHE | A | 574 | 1236 | 1437 | 1638 | −188 | 31 | 7 | | C |
| ATOM | 1543 | CE2 | PHE | A | 574 | −1.801 | −17.232 | 22.612 | 1.00 | 14.82 | | | C |
| ANISOU | 1543 | CE2 | PHE | A | 574 | 1730 | 1870 | 2032 | −148 | 29 | −3 | | C |
| ATOM | 1544 | CD2 | PHE | A | 574 | −1.465 | −18.569 | 22.697 | 1.00 | 18.96 | | | C |
| ANISOU | 1544 | CD2 | PHE | A | 574 | 2237 | 2406 | 2561 | −97 | 34 | −47 | | C |
| ATOM | 1545 | C | PHE | A | 574 | 0.261 | −22.448 | 24.569 | 1.00 | 12.50 | | | C |
| ANISOU | 1545 | C | PHE | A | 574 | 1323 | 1539 | 1889 | 107 | −16 | −126 | | C |
| ATOM | 1546 | O | PHE | A | 574 | 1.359 | −23.010 | 24.612 | 1.00 | 12.32 | | | O |
| ANISOU | 1546 | O | PHE | A | 574 | 1229 | 1531 | 1922 | 147 | −10 | −146 | | O |
| ATOM | 1547 | N | SER | A | 575 | −0.891 | −23.123 | 24.585 | 1.00 | 12.05 | | | N |
| ANISOU | 1547 | N | SER | A | 575 | 1332 | 1433 | 1815 | 123 | −15 | −123 | | N |
| ATOM | 1548 | CA | SER | A | 575 | −0.905 | −24.584 | 24.630 | 1.00 | 12.16 | | | C |
| ANISOU | 1548 | CA | SER | A | 575 | 1340 | 1395 | 1885 | 183 | −4 | −142 | | C |
| ATOM | 1549 | CB | SER | A | 575 | −0.496 | −25.122 | 26.003 | 1.00 | 15.40 | | | C |
| ANISOU | 1549 | CB | SER | A | 575 | 1733 | 1776 | 2343 | 240 | −61 | −92 | | C |
| ATOM | 1550 | OG | SER | A | 575 | −1.553 | −24.912 | 26.948 | 1.00 | 10.73 | | | O |
| ANISOU | 1550 | OG | SER | A | 575 | 1208 | 1162 | 1708 | 229 | −102 | −40 | | O |
| ATOM | 1551 | C | SER | A | 575 | −2.311 | −25.053 | 24.294 | 1.00 | 12.93 | | | C |
| ANISOU | 1551 | C | SER | A | 575 | 1510 | 1447 | 1956 | 170 | 11 | −151 | | C |
| ATOM | 1552 | O | SER | A | 575 | −3.227 | −24.250 | 24.095 | 1.00 | 11.44 | | | O |
| ANISOU | 1552 | O | SER | A | 575 | 1369 | 1273 | 1703 | 122 | 6 | −136 | | O |
| ATOM | 1553 | N | SER | A | 576 | −2.487 | −26.372 | 24.263 | 1.00 | 9.83 | | | N |
| ANISOU | 1553 | N | SER | A | 576 | 1119 | 990 | 1626 | 214 | 27 | −175 | | N |
| ATOM | 1554 | CA | SER | A | 576 | −3.836 | −26.900 | 24.085 | 1.00 | 15.74 | | | C |
| ANISOU | 1554 | CA | SER | A | 576 | 1928 | 1687 | 2364 | 198 | 36 | −184 | | C |
| ATOM | 1555 | CB | SER | A | 576 | −3.814 | −28.425 | 24.117 | 1.00 | 21.29 | | | C |
| ANISOU | 1555 | CB | SER | A | 576 | 2620 | 2301 | 3168 | 249 | 56 | −212 | | C |
| ATOM | 1556 | OG | SER | A | 576 | −3.392 | −28.883 | 25.390 | 1.00 | 24.42 | | | O |
| ANISOU | 1556 | OG | SER | A | 576 | 3004 | 2651 | 3623 | 303 | 15 | −134 | | O |
| ATOM | 1557 | C | SER | A | 576 | −4.772 | −26.369 | 25.159 | 1.00 | 10.80 | | | C |
| ANISOU | 1557 | C | SER | A | 576 | 1356 | 1050 | 1697 | 181 | −11 | −110 | | C |
| ATOM | 1558 | O | SER | A | 576 | −5.978 | −26.210 | 24.919 | 1.00 | 10.19 | | | O |
| ANISOU | 1558 | O | SER | A | 576 | 1327 | 965 | 1581 | 145 | −7 | −112 | | O |
| ATOM | 1559 | N | LYS | A | 577 | −4.242 | −26.096 | 26.350 | 1.00 | 11.70 | | | N |
| ANISOU | 1559 | N | LYS | A | 577 | 1457 | 1172 | 1815 | 207 | −56 | −49 | | N |
| ATOM | 1560 | CA | LYS | A | 577 | −5.073 | −25.601 | 27.443 | 1.00 | 8.18 | | | C |
| ANISOU | 1560 | CA | LYS | A | 577 | 1057 | 729 | 1322 | 193 | −95 | 10 | | C |
| ATOM | 1561 | CB | LYS | A | 577 | −4.358 | −25.821 | 28.784 | 1.00 | 8.62 | | | C |
| ANISOU | 1561 | CB | LYS | A | 577 | 1088 | 795 | 1392 | 239 | −143 | 72 | | C |
| ATOM | 1562 | CG | LYS | A | 577 | −3.977 | −27.287 | 29.039 | 1.00 | 9.39 | | | C |
| ANISOU | 1562 | CG | LYS | A | 577 | 1164 | 828 | 1577 | 302 | −140 | 103 | | C |
| ATOM | 1563 | CD | LYS | A | 577 | −5.231 | −28.187 | 28.971 | 1.00 | 10.79 | | | C |
| ANISOU | 1563 | CD | LYS | A | 577 | 1392 | 924 | 1783 | 296 | −111 | 117 | | C |
| ATOM | 1564 | CE | LYS | A | 577 | −4.873 | −29.602 | 29.412 | 1.00 | 13.91 | | | C |
| ANISOU | 1564 | CE | LYS | A | 577 | 1767 | 1236 | 2282 | 361 | −110 | 167 | | C |
| ATOM | 1565 | NZ | LYS | A | 577 | −5.962 | −30.564 | 29.155 | 1.00 | 15.17 | | | N |
| ANISOU | 1565 | NZ | LYS | A | 577 | 1965 | 1301 | 2500 | 348 | −73 | 165 | | N |
| ATOM | 1566 | C | LYS | A | 577 | −5.455 | −24.131 | 27.295 | 1.00 | 7.58 | | | C |
| ANISOU | 1566 | C | LYS | A | 577 | 1000 | 705 | 1174 | 139 | −104 | 4 | | C |
| ATOM | 1567 | O | LYS | A | 577 | −6.416 | −23.695 | 27.937 | 1.00 | 12.26 | | | O |
| ANISOU | 1567 | O | LYS | A | 577 | 1635 | 1296 | 1728 | 121 | −121 | 31 | | O |
| ATOM | 1568 | N | SER | A | 578 | −4.745 | −23.346 | 26.484 | 1.00 | 11.08 | | | N |
| ANISOU | 1568 | N | SER | A | 578 | 1411 | 1192 | 1607 | 112 | −88 | −29 | | N |
| ATOM | 1569 | CA | SER | A | 578 | −5.269 | −22.015 | 26.186 | 1.00 | 11.61 | | | C |
| ANISOU | 1569 | CA | SER | A | 578 | 1500 | 1286 | 1624 | 61 | −88 | −26 | | C |
| ATOM | 1570 | CB | SER | A | 578 | −4.140 | −21.065 | 25.730 | 1.00 | 7.80 | | | C |
| ANISOU | 1570 | CB | SER | A | 578 | 969 | 849 | 1147 | 34 | −81 | −38 | | C |
| ATOM | 1571 | OG | SER | A | 578 | −3.352 | −21.601 | 24.680 | 1.00 | 9.52 | | | O |
| ANISOU | 1571 | OG | SER | A | 578 | 1144 | 1090 | 1384 | 40 | −39 | −71 | | O |
| ATOM | 1572 | C | SER | A | 578 | −6.413 | −22.081 | 25.162 | 1.00 | 9.85 | | | C |
| ANISOU | 1572 | C | SER | A | 578 | 1314 | 1055 | 1373 | 34 | −58 | −40 | | C |
| ATOM | 1573 | O | SER | A | 578 | −7.345 | −21.267 | 25.230 | 1.00 | 9.16 | | | O |
| ANISOU | 1573 | O | SER | A | 578 | 1259 | 968 | 1253 | 7 | −68 | −19 | | O |
| ATOM | 1574 | N | ASP | A | 579 | −6.408 | −23.071 | 24.260 | 1.00 | 10.56 | | | N |
| ANISOU | 1574 | N | ASP | A | 579 | 1395 | 1139 | 1477 | 44 | −25 | −80 | | N |
| ATOM | 1575 | CA | ASP | A | 579 | −7.603 | −23.332 | 23.449 | 1.00 | 10.74 | | | C |
| ANISOU | 1575 | CA | ASP | A | 579 | 1450 | 1160 | 1470 | 20 | −7 | −101 | | C |
| ATOM | 1576 | CB | ASP | A | 579 | −7.335 | −24.415 | 22.399 | 1.00 | 8.92 | | | C |
| ANISOU | 1576 | CB | ASP | A | 579 | 1198 | 935 | 1258 | 27 | 33 | −172 | | C |
| ATOM | 1577 | CG | ASP | A | 579 | −6.674 | −23.881 | 21.127 | 1.00 | 13.54 | | | C |
| ANISOU | 1577 | CG | ASP | A | 579 | 1751 | 1601 | 1794 | 0 | 68 | −204 | | C |
| ATOM | 1578 | OD1 | ASP | A | 579 | −6.354 | −22.674 | 21.021 | 1.00 | 12.35 | | | O |
| ANISOU | 1578 | OD1 | ASP | A | 579 | 1593 | 1494 | 1607 | −26 | 62 | −159 | | O |
| ATOM | 1579 | OD2 | ASP | A | 579 | −6.463 | −24.706 | 20.220 | 1.00 | 16.30 | | | O |
| ANISOU | 1579 | OD2 | ASP | A | 579 | 2079 | 1968 | 2144 | 4 | 106 | −277 | | O |
| ATOM | 1580 | C | ASP | A | 579 | −8.774 | −23.773 | 24.323 | 1.00 | 9.72 | | | C |
| ANISOU | 1580 | C | ASP | A | 579 | 1360 | 979 | 1353 | 28 | −28 | −76 | | C |
| ATOM | 1581 | O | ASP | A | 579 | −9.925 | −23.399 | 24.071 | 1.00 | 7.53 | | | O |
| ANISOU | 1581 | O | ASP | A | 579 | 1109 | 709 | 1043 | 1 | −33 | −69 | | O |
| ATOM | 1582 | N | ILE | A | 580 | −8.508 | −24.622 | 25.321 | 1.00 | 6.94 | | | N |
| ANISOU | 1582 | N | ILE | A | 580 | 1008 | 579 | 1051 | 66 | −39 | −56 | | N |
| ATOM | 1583 | CA | ILE | A | 580 | −9.574 | −25.062 | 26.216 | 1.00 | 7.20 | | | C |
| ANISOU | 1583 | CA | ILE | A | 580 | 1075 | 568 | 1093 | 70 | −51 | −19 | | C |
| ATOM | 1584 | CB | ILE | A | 580 | −9.043 | −26.090 | 27.236 | 1.00 | 7.27 | | | C |
| ANISOU | 1584 | CB | ILE | A | 580 | 1076 | 529 | 1158 | 116 | −60 | 20 | | C |
| ATOM | 1585 | CG1 | ILE | A | 580 | −8.626 | −27.399 | 26.551 | 1.00 | 7.90 | | | C |
| ANISOU | 1585 | CG1 | ILE | A | 580 | 1132 | 550 | 1318 | 141 | −30 | −22 | | C |
| ATOM | 1586 | CD1 | ILE | A | 580 | −9.776 | −28.319 | 26.127 | 1.00 | 8.10 | | | C |
| ANISOU | 1586 | CD1 | ILE | A | 580 | 1179 | 514 | 1383 | 119 | −3 | −52 | | C |
| ATOM | 1587 | CG2 | ILE | A | 580 | −10.097 | −26.358 | 28.358 | 1.00 | 7.23 | | | C |
| ANISOU | 1587 | CG2 | ILE | A | 580 | 1104 | 496 | 1145 | 116 | −71 | 81 | | C |
| ATOM | 1588 | C | ILE | A | 580 | −10.209 | −23.868 | 26.916 | 1.00 | 9.40 | | | C |
| ANISOU | 1588 | C | ILE | A | 580 | 1376 | 877 | 1318 | 50 | −75 | 18 | | C |
| ATOM | 1589 | O | ILE | A | 580 | −11.436 | −23.741 | 26.976 | 1.00 | 6.55 | | | O |
| ANISOU | 1589 | O | ILE | A | 580 | 1039 | 508 | 941 | 30 | −73 | 25 | | O |
| ATOM | 1590 | N | TRP | A | 581 | −9.385 | −22.991 | 27.490 | 1.00 | 8.75 | | | N |
| ANISOU | 1590 | N | TRP | A | 581 | 1280 | 828 | 1218 | 55 | −98 | 33 | | N |
| ATOM | 1591 | CA | TRP | A | 581 | −9.919 | −21.805 | 28.143 | 1.00 | 6.00 | | | C |
| ANISOU | 1591 | CA | TRP | A | 581 | 948 | 500 | 831 | 37 | −117 | 48 | | C |
| ATOM | 1592 | CB | TRP | A | 581 | −8.764 | −20.930 | 28.638 | 1.00 | 8.97 | | | C |
| ANISOU | 1592 | CB | TRP | A | 581 | 1298 | 909 | 1202 | 37 | −141 | 43 | | C |
| ATOM | 1593 | CG | TRP | A | 581 | −9.214 | −19.644 | 29.291 | 1.00 | 8.61 | | | C |
| ANISOU | 1593 | CG | TRP | A | 581 | 1265 | 876 | 1132 | 16 | −157 | 38 | | C |
| ATOM | 1594 | CD1 | TRP | A | 581 | −9.718 | −18.546 | 28.664 | 1.00 | 6.75 | | | C |
| ANISOU | 1594 | CD1 | TRP | A | 581 | 1035 | 631 | 901 | −14 | −147 | 29 | | C |
| ATOM | 1595 | NE1 | TRP | A | 581 | −9.999 | −17.566 | 29.575 | 1.00 | 7.17 | | | N |
| ANISOU | 1595 | NE1 | TRP | A | 581 | 1094 | 684 | 947 | −22 | −163 | 14 | | N |
| ATOM | 1596 | CE2 | TRP | A | 581 | −9.668 | −18.018 | 30.822 | 1.00 | 9.38 | | | C |
| ANISOU | 1596 | CE2 | TRP | A | 581 | 1373 | 993 | 1199 | −1 | −185 | 10 | | C |
| ATOM | 1597 | CD2 | TRP | A | 581 | −9.152 | −19.317 | 30.678 | 1.00 | 10.76 | | | C |
| ANISOU | 1597 | CD2 | TRP | A | 581 | 1539 | 1172 | 1376 | 25 | −185 | 37 | | C |
| ATOM | 1598 | CE3 | TRP | A | 581 | −8.727 | −20.007 | 31.823 | 1.00 | 9.02 | | | C |
| ANISOU | 1598 | CE3 | TRP | A | 581 | 1314 | 983 | 1130 | 55 | −210 | 58 | | C |
| ATOM | 1599 | CZ3 | TRP | A | 581 | −8.820 | −19.375 | 33.052 | 1.00 | 10.59 | | | C |
| ANISOU | 1599 | CZ3 | TRP | A | 581 | 1516 | 1227 | 1282 | 52 | −235 | 43 | | C |
| ATOM | 1600 | CH2 | TRP | A | 581 | −9.334 | −18.077 | 33.160 | 1.00 | 8.16 | | | C |
| ANISOU | 1600 | CH2 | TRP | A | 581 | 1216 | 915 | 970 | 22 | −230 | −5 | | C |
| ATOM | 1601 | CZ2 | TRP | A | 581 | −9.757 | −17.382 | 32.058 | 1.00 | 13.53 | | | C |
| ANISOU | 1601 | CZ2 | TRP | A | 581 | 1900 | 1545 | 1695 | −2 | −205 | −18 | | C |
| ATOM | 1602 | C | TRP | A | 581 | −10.819 | −21.017 | 27.198 | 1.00 | 8.10 | | | C |
| ANISOU | 1602 | C | TRP | A | 581 | 1225 | 771 | 1079 | 3 | −104 | 36 | | C |
| ATOM | 1603 | O | TRP | A | 581 | −11.922 | −20.598 | 27.570 | 1.00 | 7.80 | | | O |
| ANISOU | 1603 | O | TRP | A | 581 | 1208 | 726 | 1028 | −5 | −106 | 47 | | O |
| ATOM | 1604 | N | ALA | A | 582 | −10.336 | −20.770 | 25.974 | 1.00 | 5.79 | | | N |
| ANISOU | 1604 | N | ALA | A | 582 | 914 | 499 | 785 | −14 | −89 | 19 | | N |
| ATOM | 1605 | CA | ALA | A | 582 | −11.101 | −19.982 | 25.018 | 1.00 | 6.09 | | | C |
| ANISOU | 1605 | CA | ALA | A | 582 | 959 | 555 | 800 | −42 | −83 | 26 | | C |
| ATOM | 1606 | CB | ALA | A | 582 | −10.260 | −19.736 | 23.759 | 1.00 | 6.93 | | | C |
| ANISOU | 1606 | CB | ALA | A | 582 | 1039 | 703 | 891 | −61 | −63 | 18 | | C |
| ATOM | 1607 | C | ALA | A | 582 | −12.421 | −20.666 | 24.671 | 1.00 | 6.32 | | | C |
| ANISOU | 1607 | C | ALA | A | 582 | 1005 | 578 | 820 | −47 | −78 | 19 | | C |
| ATOM | 1608 | O | ALA | A | 582 | −13.456 | −20.000 | 24.513 | 1.00 | 6.62 | | | O |
| ANISOU | 1608 | O | ALA | A | 582 | 1050 | 621 | 845 | −59 | −86 | 38 | | O |
| ATOM | 1609 | N | PHE | A | 583 | −12.414 | −21.995 | 24.572 | 1.00 | 6.65 | | | N |
| ANISOU | 1609 | N | PHE | A | 583 | 1045 | 600 | 879 | −37 | −64 | −11 | | N |
| ATOM | 1610 | CA | PHE | A | 583 | −13.656 | −22.726 | 24.345 | 1.00 | 9.28 | | | C |
| ANISOU | 1610 | CA | PHE | A | 583 | 1389 | 920 | 1219 | −49 | −60 | −27 | | C |
| ATOM | 1611 | CB | PHE | A | 583 | −13.378 | −24.219 | 24.203 | 1.00 | 6.26 | | | C |
| ANISOU | 1611 | CB | PHE | A | 583 | 1000 | 498 | 880 | −40 | −39 | −68 | | C |
| ATOM | 1612 | CG | PHE | A | 583 | −14.642 | −25.049 | 24.040 | 1.00 | 6.46 | | | C |
| ANISOU | 1612 | CG | PHE | A | 583 | 1030 | 497 | 929 | −61 | −32 | −91 | | C |
| ATOM | 1613 | CD1 | PHE | A | 583 | −15.259 | −25.156 | 22.798 | 1.00 | 11.12 | | | C |
| ANISOU | 1613 | CD1 | PHE | A | 583 | 1605 | 1129 | 1491 | −94 | −31 | −144 | | C |
| ATOM | 1614 | CE1 | PHE | A | 583 | −16.424 | −25.915 | 22.640 | 1.00 | 13.62 | | | C |
| ANISOU | 1614 | CE1 | PHE | A | 583 | 1915 | 1424 | 1835 | −121 | −29 | −176 | | C |
| ATOM | 1615 | CZ | PHE | A | 583 | −16.980 | −26.566 | 23.740 | 1.00 | 10.07 | | | C |
| ANISOU | 1615 | CZ | PHE | A | 583 | 1476 | 903 | 1447 | −117 | −19 | −144 | | C |
| ATOM | 1616 | CE2 | PHE | A | 583 | −16.357 | −26.469 | 24.990 | 1.00 | 9.99 | | | C |
| ANISOU | 1616 | CE2 | PHE | A | 583 | 1485 | 856 | 1455 | −81 | −17 | −80 | | C |
| ATOM | 1617 | CD2 | PHE | A | 583 | −15.191 | −25.711 | 25.124 | 1.00 | 6.52 | | | C |
| ANISOU | 1617 | CD2 | PHE | A | 583 | 1050 | 446 | 982 | −53 | −27 | −59 | | C |
| ATOM | 1618 | C | PHE | A | 583 | −14.655 | −22.487 | 25.472 | 1.00 | 10.18 | | | C |
| ANISOU | 1618 | C | PHE | A | 583 | 1519 | 1008 | 1340 | −45 | −69 | 8 | | C |
| ATOM | 1619 | O | PHE | A | 583 | −15.851 | −22.299 | 25.219 | 1.00 | 8.37 | | | O |
| ANISOU | 1619 | O | PHE | A | 583 | 1288 | 788 | 1103 | −62 | −73 | 10 | | O |
| ATOM | 1620 | N | GLY | A | 584 | −14.186 | −22.511 | 26.723 | 1.00 | 11.14 | | | N |
| ANISOU | 1620 | N | GLY | A | 584 | 1651 | 1111 | 1473 | −22 | −74 | 35 | | N |
| ATOM | 1621 | CA | GLY | A | 584 | −15.067 | −22.174 | 27.830 | 1.00 | 9.03 | | | C |
| ANISOU | 1621 | CA | GLY | A | 584 | 1396 | 839 | 1197 | −19 | −76 | 63 | | C |
| ATOM | 1622 | C | GLY | A | 584 | −15.695 | −20.803 | 27.663 | 1.00 | 8.45 | | | C |
| ANISOU | 1622 | C | GLY | A | 584 | 1319 | 787 | 1104 | −29 | −86 | 63 | | C |
| ATOM | 1623 | O | GLY | A | 584 | −16.892 | −20.624 | 27.912 | 1.00 | 7.82 | | | O |
| ANISOU | 1623 | O | GLY | A | 584 | 1238 | 707 | 1026 | −35 | −80 | 69 | | O |
| ATOM | 1624 | N | VAL | A | 585 | −14.895 | −19.811 | 27.251 | 1.00 | 5.27 | | | N |
| ANISOU | 1624 | N | VAL | A | 585 | 909 | 398 | 695 | −31 | −97 | 61 | | N |
| ATOM | 1625 | CA | VAL | A | 585 | −15.442 | −18.479 | 27.003 | 1.00 | 7.17 | | | C |
| ANISOU | 1625 | CA | VAL | A | 585 | 1144 | 641 | 940 | −38 | −105 | 70 | | C |
| ATOM | 1626 | CB | VAL | A | 585 | −14.299 | −17.466 | 26.733 | 1.00 | 6.51 | | | C |
| ANISOU | 1626 | CB | VAL | A | 585 | 1051 | 558 | 866 | −45 | −113 | 73 | | C |
| ATOM | 1627 | CG1 | VAL | A | 585 | −14.839 | −16.074 | 26.393 | 1.00 | 5.49 | | | C |
| ANISOU | 1627 | CG1 | VAL | A | 585 | 913 | 411 | 762 | −51 | −118 | 95 | | C |
| ATOM | 1628 | CG2 | VAL | A | 585 | −13.353 | −17.358 | 27.958 | 1.00 | 5.39 | | | C |
| ANISOU | 1628 | CG2 | VAL | A | 585 | 911 | 412 | 725 | −35 | −121 | 53 | | C |
| ATOM | 1629 | C | VAL | A | 585 | −16.442 | −18.536 | 25.850 | 1.00 | 6.62 | | | C |
| ANISOU | 1629 | C | VAL | A | 585 | 1062 | 587 | 865 | −52 | −106 | 80 | | C |
| ATOM | 1630 | O | VAL | A | 585 | −17.500 | −17.896 | 25.894 | 1.00 | 8.05 | | | O |
| ANISOU | 1630 | O | VAL | A | 585 | 1235 | 766 | 1059 | −48 | −111 | 94 | | O |
| ATOM | 1631 | N | ALEU | A | 586 | −16.123 | −19.302 | 24.804 | 0.74 | 5.42 | | | N |
| ANISOU | 1631 | N | ALEU | A | 586 | 905 | 460 | 695 | −65 | −103 | 67 | | N |
| ATOM | 1632 | CA | ALEU | A | 586 | −17.075 | −19.451 | 23.707 | 0.74 | 8.53 | | | C |
| ANISOU | 1632 | CA | ALEU | A | 586 | 1283 | 890 | 1069 | −82 | −111 | 67 | | C |
| ATOM | 1633 | CB | ALEU | A | 586 | −16.481 | −20.325 | 22.606 | 0.74 | 7.16 | | | C |
| ANISOU | 1633 | CB | ALEU | A | 586 | 1103 | 753 | 866 | −99 | −103 | 31 | | C |
| ATOM | 1634 | CG | ALEU | A | 586 | −17.455 | −20.727 | 21.498 | 0.74 | 9.81 | | | C |
| ANISOU | 1634 | CG | ALEU | A | 586 | 1418 | 1142 | 1167 | −123 | −115 | 9 | | C |
| ATOM | 1635 | CD1 | ALEU | A | 586 | −17.876 | −19.508 | 20.709 | 0.74 | 6.52 | | | C |
| ANISOU | 1635 | CD1 | ALEU | A | 586 | 986 | 779 | 714 | −126 | −140 | 66 | | C |
| ATOM | 1636 | CD2 | ALEU | A | 586 | −16.802 | −21.764 | 20.585 | 0.74 | 9.34 | | | C |
| ANISOU | 1636 | CD2 | ALEU | A | 586 | 1352 | 1116 | 1082 | −140 | −99 | −56 | | C |
| ATOM | 1637 | C | ALEU | A | 586 | −18.396 | −20.033 | 24.200 | 0.74 | 8.52 | | | C |
| ANISOU | 1637 | C | ALEU | A | 586 | 1276 | 875 | 1087 | −84 | −110 | 56 | | C |
| ATOM | 1638 | O | ALEU | A | 586 | −19.471 | −19.581 | 23.792 | 0.74 | 6.69 | | | O |
| ANISOU | 1638 | O | ALEU | A | 586 | 1022 | 666 | 853 | −88 | −126 | 72 | | O |
| ATOM | 1639 | N | BLEU | A | 586 | −16.134 | −19.327 | 24.820 | 0.26 | 7.55 | | | N |
| ANISOU | 1639 | N | BLEU | A | 586 | 1175 | 729 | 965 | −65 | −103 | 67 | | N |
| ATOM | 1640 | CA | BLEU | A | 586 | −17.058 | −19.466 | 23.698 | 0.26 | 8.21 | | | C |
| ANISOU | 1640 | CA | BLEU | A | 586 | 1242 | 849 | 1028 | −82 | −111 | 67 | | C |
| ATOM | 1641 | CB | BLEU | A | 586 | −16.407 | −20.299 | 22.592 | 0.26 | 8.11 | | | C |
| ANISOU | 1641 | CB | BLEU | A | 586 | 1223 | 874 | 985 | −99 | −103 | 31 | | C |
| ATOM | 1642 | CG | BLEU | A | 586 | −17.093 | −20.403 | 21.227 | 0.26 | 9.85 | | | C |
| ANISOU | 1642 | CG | BLEU | A | 586 | 1423 | 1162 | 1158 | −123 | −116 | 20 | | C |
| ATOM | 1643 | CD1 | BLEU | A | 586 | −16.076 | −20.797 | 20.152 | 0.26 | 8.17 | | | C |
| ANISOU | 1643 | CD1 | BLEU | A | 586 | 1202 | 1001 | 899 | −137 | −99 | −14 | | C |
| ATOM | 1644 | CD2 | BLEU | A | 586 | −18.220 | −21.414 | 21.275 | 0.26 | 9.07 | | | C |
| ANISOU | 1644 | CD2 | BLEU | A | 586 | 1314 | 1057 | 1076 | −137 | −119 | −23 | | C |
| ATOM | 1645 | C | BLEU | A | 586 | −18.384 | −20.078 | 24.146 | 0.26 | 8.06 | | | C |
| ANISOU | 1645 | C | BLEU | A | 586 | 1217 | 818 | 1027 | −85 | −110 | 55 | | C |
| ATOM | 1646 | O | BLEU | A | 586 | −19.451 | −19.680 | 23.664 | 0.26 | 7.43 | | | O |
| ANISOU | 1646 | O | BLEU | A | 586 | 1116 | 764 | 944 | −91 | −126 | 68 | | O |
| ATOM | 1647 | N | MET | A | 587 | −18.342 | −21.046 | 25.067 | 1.00 | 6.50 | | | N |
| ANISOU | 1647 | N | MET | A | 587 | 1033 | 584 | 854 | −81 | −92 | 39 | | N |
| ATOM | 1648 | CA | MET | A | 587 | −19.592 | −21.602 | 25.596 | 1.00 | 11.01 | | | C |
| ANISOU | 1648 | CA | MET | A | 587 | 1594 | 1141 | 1449 | −90 | −82 | 37 | | C |
| ATOM | 1649 | CB | MET | A | 587 | −19.332 | −22.760 | 26.573 | 1.00 | 7.39 | | | C |
| ANISOU | 1649 | CB | MET | A | 587 | 1153 | 638 | 1018 | −88 | −57 | 38 | | C |
| ATOM | 1650 | CG | MET | A | 587 | −18.824 | −24.049 | 25.955 | 1.00 | 11.38 | | | C |
| ANISOU | 1650 | CG | MET | A | 587 | 1658 | 1116 | 1550 | −101 | −48 | 4 | | C |
| ATOM | 1651 | SD | MET | A | 587 | −18.788 | −25.378 | 27.186 | 1.00 | 14.58 | | | S |
| ANISOU | 1651 | SD | MET | A | 587 | 2078 | 1452 | 2010 | −95 | −18 | 34 | | S |
| ATOM | 1652 | CE | MET | A | 587 | −20.522 | −25.466 | 27.683 | 1.00 | 10.52 | | | C |
| ANISOU | 1652 | CE | MET | A | 587 | 1544 | 939 | 1514 | −124 | −0 | 51 | | C |
| ATOM | 1653 | C | MET | A | 587 | −20.380 | −20.518 | 26.306 | 1.00 | 9.23 | | | C |
| ANISOU | 1653 | C | MET | A | 587 | 1359 | 919 | 1228 | −75 | −84 | 63 | | C |
| ATOM | 1654 | O | MET | A | 587 | −21.612 | −20.440 | 26.187 | 1.00 | 6.70 | | | O |
| ANISOU | 1654 | O | MET | A | 587 | 1012 | 613 | 922 | −82 | −85 | 64 | | O |
| ATOM | 1655 | N | TRP | A | 588 | −19.676 | −19.660 | 27.040 | 1.00 | 5.52 | | | N |
| ANISOU | 1655 | N | TRP | A | 588 | 906 | 438 | 753 | −53 | −83 | 74 | | N |
| ATOM | 1656 | CA | TRP | A | 588 | −20.323 | −18.542 | 27.712 | 1.00 | 5.60 | | | C |
| ANISOU | 1656 | CA | TRP | A | 588 | 906 | 445 | 777 | −35 | −80 | 81 | | C |
| ATOM | 1657 | CB | TRP | A | 588 | −19.304 | −17.830 | 28.599 | 1.00 | 6.03 | | | C |
| ANISOU | 1657 | CB | TRP | A | 588 | 981 | 486 | 825 | −20 | −78 | 71 | | C |
| ATOM | 1658 | CG | TRP | A | 588 | −19.862 | −16.718 | 29.438 | 1.00 | 6.17 | | | C |
| ANISOU | 1658 | CG | TRP | A | 588 | 989 | 495 | 862 | −1 | −68 | 54 | | C |
| ATOM | 1659 | CD1 | TRP | A | 588 | −20.240 | −16.789 | 30.766 | 1.00 | 5.97 | | | C |
| ANISOU | 1659 | CD1 | TRP | A | 588 | 967 | 483 | 819 | 8 | −43 | 31 | | C |
| ATOM | 1660 | NE1 | TRP | A | 588 | −20.691 | −15.566 | 31.185 | 1.00 | 8.35 | | | N |
| ANISOU | 1660 | NE1 | TRP | A | 588 | 1252 | 769 | 1151 | 26 | −34 | −1 | | N |
| ATOM | 1661 | CE2 | TRP | A | 588 | −20.617 | −14.676 | 30.141 | 1.00 | 7.81 | | | C |
| ANISOU | 1661 | CE2 | TRP | A | 588 | 1170 | 665 | 1133 | 30 | −56 | 16 | | C |
| ATOM | 1662 | CD2 | TRP | A | 588 | −20.078 | −15.366 | 29.029 | 1.00 | 5.96 | | | C |
| ANISOU | 1662 | CD2 | TRP | A | 588 | 945 | 441 | 880 | 11 | −78 | 55 | | C |
| ATOM | 1663 | CE3 | TRP | A | 588 | −19.892 | −14.673 | 27.815 | 1.00 | 7.21 | | | C |
| ANISOU | 1663 | CE3 | TRP | A | 588 | 1092 | 583 | 1066 | 9 | −101 | 91 | | C |
| ATOM | 1664 | CZ3 | TRP | A | 588 | −20.237 | −13.324 | 27.760 | 1.00 | 6.44 | | | C |
| ANISOU | 1664 | CZ3 | TRP | A | 588 | 974 | 442 | 1031 | 28 | −103 | 99 | | C |
| ATOM | 1665 | CH2 | TRP | A | 588 | −20.759 | −12.659 | 28.899 | 1.00 | 9.20 | | | C |
| ANISOU | 1665 | CH2 | TRP | A | 588 | 1314 | 764 | 1417 | 50 | −81 | 50 | | C |
| ATOM | 1666 | CZ2 | TRP | A | 588 | −20.935 | −13.315 | 30.099 | 1.00 | 9.20 | | | C |
| ANISOU | 1666 | CZ2 | TRP | A | 588 | 1325 | 797 | 1374 | 50 | −56 | 2 | | C |
| ATOM | 1667 | C | TRP | A | 588 | −20.953 | −17.573 | 26.720 | 1.00 | 6.86 | | | C |
| ANISOU | 1667 | C | TRP | A | 588 | 1038 | 614 | 954 | −31 | −101 | 99 | | C |
| ATOM | 1668 | O | TRP | A | 588 | −22.087 | −17.132 | 26.920 | 1.00 | 8.35 | | | O |
| ANISOU | 1668 | O | TRP | A | 588 | 1199 | 804 | 1170 | −19 | −98 | 103 | | O |
| ATOM | 1669 | N | GLU | A | 589 | −20.235 | −17.224 | 25.642 | 1.00 | 5.78 | | | N |
| ANISOU | 1669 | N | GLU | A | 589 | 903 | 489 | 804 | −38 | −122 | 118 | | N |
| ATOM | 1670 | CA | GLU | A | 589 | −20.823 | −16.373 | 24.608 | 1.00 | 6.12 | | | C |
| ANISOU | 1670 | CA | GLU | A | 589 | 918 | 551 | 855 | −33 | −147 | 158 | | C |
| ATOM | 1671 | CB | GLU | A | 589 | −19.836 | −16.165 | 23.457 | 1.00 | 8.88 | | | C |
| ANISOU | 1671 | CB | GLU | A | 589 | 1275 | 930 | 1171 | −48 | −160 | 186 | | C |
| ATOM | 1672 | CG | GLU | A | 589 | −18.566 | −15.407 | 23.779 | 1.00 | 14.50 | | | C |
| ANISOU | 1672 | CG | GLU | A | 589 | 2006 | 1605 | 1900 | −47 | −150 | 195 | | C |
| ATOM | 1673 | CD | GLU | A | 589 | −17.704 | −15.223 | 22.514 | 1.00 | 13.03 | | | C |
| ANISOU | 1673 | CD | GLU | A | 589 | 1816 | 1459 | 1675 | −67 | −155 | 232 | | C |
| ATOM | 1674 | OE1 | GLU | A | 589 | −16.907 | −16.132 | 22.190 | 1.00 | 9.62 | | | O |
| ANISOU | 1674 | OE1 | GLU | A | 589 | 1394 | 1060 | 1203 | −84 | −143 | 199 | | O |
| ATOM | 1675 | OE2 | GLU | A | 589 | −17.850 | −14.182 | 21.839 | 1.00 | 9.21 | | | O |
| ANISOU | 1675 | OE2 | GLU | A | 589 | 1318 | 977 | 1205 | −65 | −167 | 297 | | O |
| ATOM | 1676 | C | GLU | A | 589 | −22.101 | −16.985 | 24.043 | 1.00 | 6.84 | | | C |
| ANISOU | 1676 | C | GLU | A | 589 | 976 | 685 | 938 | −43 | −161 | 156 | | C |
| ATOM | 1677 | O | GLU | A | 589 | −23.082 | −16.277 | 23.782 | 1.00 | 9.22 | | | O |
| ANISOU | 1677 | O | GLU | A | 589 | 1241 | 995 | 1266 | −25 | −179 | 186 | | O |
| ATOM | 1678 | N | ILE | A | 590 | −22.080 | −18.293 | 23.776 | 1.00 | 6.32 | | | N |
| ANISOU | 1678 | N | ILE | A | 590 | 915 | 643 | 845 | −71 | −156 | 118 | | N |
| ATOM | 1679 | CA | ILE | A | 590 | −23.244 | −18.940 | 23.174 | 1.00 | 6.70 | | | C |
| ANISOU | 1679 | CA | ILE | A | 590 | 924 | 734 | 889 | −91 | −172 | 102 | | C |
| ATOM | 1680 | CB | ILE | A | 590 | −22.920 | −20.402 | 22.819 | 1.00 | 7.31 | | | C |
| ANISOU | 1680 | CB | ILE | A | 590 | 1011 | 819 | 947 | −128 | −161 | 46 | | C |
| ATOM | 1681 | CG1 | ILE | A | 590 | −21.972 | −20.477 | 21.608 | 1.00 | 6.88 | | | C |
| ANISOU | 1681 | CG1 | ILE | A | 590 | 966 | 811 | 835 | −141 | −176 | 34 | | C |
| ATOM | 1682 | CD1 | ILE | A | 590 | −21.356 | −21.872 | 21.379 | 1.00 | 10.60 | | | C |
| ANISOU | 1682 | CD1 | ILE | A | 590 | 1452 | 1270 | 1305 | −167 | −154 | −36 | | C |
| ATOM | 1683 | CG2 | ILE | A | 590 | −24.205 | −21.170 | 22.560 | 1.00 | 7.19 | | | C |
| ANISOU | 1683 | CG2 | ILE | A | 590 | 953 | 830 | 951 | −157 | −170 | 13 | | C |
| ATOM | 1684 | C | ILE | A | 590 | −24.439 | −18.844 | 24.117 | 1.00 | 8.88 | | | C |
| ANISOU | 1684 | C | ILE | A | 590 | 1172 | 990 | 1214 | −79 | −157 | 101 | | C |
| ATOM | 1685 | O | ILE | A | 590 | −25.543 | −18.460 | 23.711 | 1.00 | 7.87 | | | O |
| ANISOU | 1685 | O | ILE | A | 590 | 994 | 895 | 1102 | −73 | −180 | 117 | | O |
| ATOM | 1686 | N | TYR | A | 591 | −24.243 | −19.211 | 25.389 | 1.00 | 8.38 | | | N |
| ANISOU | 1686 | N | TYR | A | 591 | 1132 | 881 | 1169 | −75 | −118 | 86 | | N |
| ATOM | 1687 | CA | TYR | A | 591 | −25.361 | −19.182 | 26.326 | 1.00 | 8.27 | | | C |
| ANISOU | 1687 | CA | TYR | A | 591 | 1089 | 861 | 1192 | −68 | −91 | 83 | | C |
| ATOM | 1688 | CB | TYR | A | 591 | −25.055 | −20.077 | 27.540 | 1.00 | 7.84 | | | C |
| ANISOU | 1688 | CB | TYR | A | 591 | 1065 | 777 | 1135 | −80 | −47 | 72 | | C |
| ATOM | 1689 | CG | TYR | A | 591 | −25.385 | −21.536 | 27.244 | 1.00 | 11.99 | | | C |
| ANISOU | 1689 | CG | TYR | A | 591 | 1582 | 1299 | 1675 | −124 | −37 | 55 | | C |
| ATOM | 1690 | CD1 | TYR | A | 591 | −24.447 | −22.370 | 26.663 | 1.00 | 8.44 | | | C |
| ANISOU | 1690 | CD1 | TYR | A | 591 | 1162 | 831 | 1214 | −141 | −46 | 39 | | C |
| ATOM | 1691 | CE1 | TYR | A | 591 | −24.738 | −23.699 | 26.377 | 1.00 | 9.13 | | | C |
| ANISOU | 1691 | CE1 | TYR | A | 591 | 1238 | 896 | 1334 | −182 | −35 | 12 | | C |
| ATOM | 1692 | CZ | TYR | A | 591 | −25.991 | −24.194 | 26.640 | 1.00 | 7.56 | | | C |
| ANISOU | 1692 | CZ | TYR | A | 591 | 997 | 698 | 1178 | −212 | −16 | 8 | | C |
| ATOM | 1693 | OH | TYR | A | 591 | −26.272 | −25.501 | 26.349 | 1.00 | 12.12 | | | O |
| ANISOU | 1693 | OH | TYR | A | 591 | 1560 | 1240 | 1804 | −259 | −3 | −25 | | O |
| ATOM | 1694 | CE2 | TYR | A | 591 | −26.961 | −23.388 | 27.204 | 1.00 | 7.67 | | | C |
| ANISOU | 1694 | CE2 | TYR | A | 591 | 976 | 742 | 1195 | −197 | −6 | 29 | | C |
| ATOM | 1695 | CD2 | TYR | A | 591 | −26.657 | −22.060 | 27.499 | 1.00 | 7.33 | | | C |
| ANISOU | 1695 | CD2 | TYR | A | 591 | 946 | 717 | 1121 | −149 | −16 | 50 | | C |
| ATOM | 1696 | C | TYR | A | 591 | −25.728 | −17.766 | 26.762 | 1.00 | 9.32 | | | C |
| ANISOU | 1696 | C | TYR | A | 591 | 1205 | 984 | 1354 | −26 | −90 | 100 | | C |
| ATOM | 1697 | O | TYR | A | 591 | −26.808 | −17.565 | 27.331 | 1.00 | 10.78 | | | O |
| ANISOU | 1697 | O | TYR | A | 591 | 1348 | 1174 | 1573 | −14 | −70 | 93 | | O |
| ATOM | 1698 | N | SER | A | 592 | −24.875 | −16.790 | 26.479 | 1.00 | 7.40 | | | N |
| ANISOU | 1698 | N | SER | A | 592 | 984 | 720 | 1109 | −4 | −108 | 119 | | N |
| ATOM | 1699 | CA | SER | A | 592 | −25.167 | −15.380 | 26.659 | 1.00 | 7.09 | | | C |
| ANISOU | 1699 | CA | SER | A | 592 | 924 | 652 | 1119 | 36 | −111 | 135 | | C |
| ATOM | 1700 | CB | SER | A | 592 | −23.905 | −14.642 | 27.086 | 1.00 | 9.27 | | | C |
| ANISOU | 1700 | CB | SER | A | 592 | 1243 | 883 | 1396 | 45 | −104 | 128 | | C |
| ATOM | 1701 | OG | SER | A | 592 | −23.452 | −15.145 | 28.324 | 1.00 | 10.60 | | | O |
| ANISOU | 1701 | OG | SER | A | 592 | 1442 | 1048 | 1538 | 38 | −71 | 84 | | O |
| ATOM | 1702 | C | SER | A | 592 | −25.730 | −14.728 | 25.405 | 1.00 | 7.54 | | | C |
| ANISOU | 1702 | C | SER | A | 592 | 939 | 729 | 1197 | 51 | −156 | 188 | | C |
| ATOM | 1703 | O | SER | A | 592 | −25.873 | −13.503 | 25.373 | 1.00 | 7.92 | | | O |
| ANISOU | 1703 | O | SER | A | 592 | 969 | 739 | 1302 | 88 | −163 | 218 | | O |
| ATOM | 1704 | N | LEU | A | 593 | −26.036 | −15.510 | 24.374 | 1.00 | 7.64 | | | N |
| ANISOU | 1704 | N | LEU | A | 593 | 933 | 802 | 1167 | 23 | −187 | 200 | | N |
| ATOM | 1705 | CA | LEU | A | 593 | −26.566 | −14.975 | 23.113 | 1.00 | 8.22 | | | C |
| ANISOU | 1705 | CA | LEU | A | 593 | 963 | 923 | 1238 | 34 | −238 | 259 | | C |
| ATOM | 1706 | CB | LEU | A | 593 | −28.014 | −14.509 | 23.294 | 1.00 | 10.17 | | | C |
| ANISOU | 1706 | CB | LEU | A | 593 | 1138 | 1177 | 1548 | 68 | −249 | 273 | | C |
| ATOM | 1707 | CG | LEU | A | 593 | −28.937 | −15.601 | 23.856 | 1.00 | 11.10 | | | C |
| ANISOU | 1707 | CG | LEU | A | 593 | 1225 | 1323 | 1669 | 40 | −225 | 214 | | C |
| ATOM | 1708 | CD1 | LEU | A | 593 | −30.369 | −15.068 | 24.052 | 1.00 | 14.92 | | | C |
| ANISOU | 1708 | CD1 | LEU | A | 593 | 1626 | 1820 | 2225 | 77 | −231 | 226 | | C |
| ATOM | 1709 | CD2 | LEU | A | 593 | −28.936 | −16.840 | 22.957 | 1.00 | 9.50 | | | C |
| ANISOU | 1709 | CD2 | LEU | A | 593 | 1020 | 1189 | 1403 | −15 | −252 | 189 | | C |
| ATOM | 1710 | C | LEU | A | 593 | −25.676 | −13.859 | 22.551 | 1.00 | 9.02 | | | C |
| ANISOU | 1710 | C | LEU | A | 593 | 1087 | 998 | 1343 | 54 | −255 | 324 | | C |
| ATOM | 1711 | O | LEU | A | 593 | −26.146 | −12.864 | 21.995 | 1.00 | 9.00 | | | O |
| ANISOU | 1711 | O | LEU | A | 593 | 1047 | 992 | 1379 | 87 | −285 | 394 | | O |
| ATOM | 1712 | N | GLY | A | 594 | −24.365 | −14.047 | 22.650 | 1.00 | 10.31 | | | N |
| ANISOU | 1712 | N | GLY | A | 594 | 1305 | 1140 | 1472 | 32 | −235 | 308 | | N |
| ATOM | 1713 | CA | GLY | A | 594 | −23.448 | −13.148 | 21.983 | 1.00 | 8.94 | | | C |
| ANISOU | 1713 | CA | GLY | A | 594 | 1148 | 952 | 1296 | 35 | −247 | 371 | | C |
| ATOM | 1714 | C | GLY | A | 594 | −23.028 | −11.922 | 22.765 | 1.00 | 12.18 | | | C |
| ANISOU | 1714 | C | GLY | A | 594 | 1571 | 1265 | 1794 | 63 | −225 | 383 | | C |
| ATOM | 1715 | O | GLY | A | 594 | −22.448 | −11.005 | 22.174 | 1.00 | 12.50 | | | O |
| ANISOU | 1715 | O | GLY | A | 594 | 1613 | 1278 | 1857 | 67 | −234 | 451 | | O |
| ATOM | 1716 | N | LYS | A | 595 | −23.300 | −11.862 | 24.066 | 1.00 | 11.77 | | | N |
| ANISOU | 1716 | N | LYS | A | 595 | 1522 | 1160 | 1789 | 79 | −193 | 317 | | N |
| ATOM | 1717 | CA | LYS | A | 595 | −22.780 | −10.751 | 24.854 | 1.00 | 14.20 | | | C |
| ANISOU | 1717 | CA | LYS | A | 595 | 1842 | 1378 | 2175 | 98 | −171 | 300 | | C |
| ATOM | 1718 | CB | LYS | A | 595 | −23.273 | −10.819 | 26.307 | 1.00 | 9.51 | | | C |
| ANISOU | 1718 | CB | LYS | A | 595 | 1246 | 758 | 1610 | 115 | −135 | 212 | | C |
| ATOM | 1719 | CG | LYS | A | 595 | −24.727 | −10.445 | 26.500 | 1.00 | 14.14 | | | C |
| ANISOU | 1719 | CG | LYS | A | 595 | 1779 | 1337 | 2258 | 157 | −131 | 212 | | C |
| ATOM | 1720 | CD | LYS | A | 595 | −25.085 | −10.449 | 27.993 | 1.00 | 24.33 | | | C |
| ANISOU | 1720 | CD | LYS | A | 595 | 3067 | 2611 | 3565 | 170 | −83 | 118 | | C |
| ATOM | 1721 | CE | LYS | A | 595 | −26.572 | −10.211 | 28.203 | 0.00 | 22.53 | | | C |
| ANISOU | 1721 | CE | LYS | A | 595 | 2776 | 2388 | 3396 | 210 | −70 | 110 | | C |
| ATOM | 1722 | NZ | LYS | A | 595 | −27.400 | −11.283 | 27.583 | 0.00 | 20.54 | | | N |
| ANISOU | 1722 | NZ | LYS | A | 595 | 2495 | 2216 | 3094 | 193 | −90 | 143 | | N |
| ATOM | 1723 | C | LYS | A | 595 | −21.256 | −10.771 | 24.841 | 1.00 | 9.57 | | | C |
| ANISOU | 1723 | C | LYS | A | 595 | 1298 | 779 | 1558 | 64 | −162 | 291 | | C |
| ATOM | 1724 | O | LYS | A | 595 | −20.626 | −11.824 | 24.721 | 1.00 | 12.58 | | | O |
| ANISOU | 1724 | O | LYS | A | 595 | 1705 | 1213 | 1862 | 34 | −160 | 268 | | O |
| ATOM | 1725 | N | AMET | A | 596 | −20.667 | −9.590 | 24.960 | 0.65 | 8.24 | | | N |
| ANISOU | 1725 | N | AMET | A | 596 | 1132 | 534 | 1466 | 69 | −156 | 308 | | N |
| ATOM | 1726 | CA | AMET | A | 596 | −19.234 | −9.499 | 25.179 | 0.65 | 9.41 | | | C |
| ANISOU | 1726 | CA | AMET | A | 596 | 1309 | 663 | 1604 | 35 | −144 | 284 | | C |
| ATOM | 1727 | CB | AMET | A | 596 | −18.796 | −8.037 | 25.116 | 0.65 | 10.89 | | | C |
| ANISOU | 1727 | CB | AMET | A | 596 | 1483 | 782 | 1872 | 36 | −130 | 299 | | C |
| ATOM | 1728 | CG | AMET | A | 596 | −17.320 | −7.801 | 25.414 | 0.65 | 13.06 | | | C |
| ANISOU | 1728 | CG | AMET | A | 596 | 1775 | 1038 | 2151 | −2 | −116 | 265 | | C |
| ATOM | 1729 | SD | AMET | A | 596 | −16.235 | −8.108 | 24.010 | 0.65 | 16.86 | | | S |
| ANISOU | 1729 | SD | AMET | A | 596 | 2260 | 1573 | 2574 | −42 | −122 | 353 | | S |
| ATOM | 1730 | CE | AMET | A | 596 | −16.463 | −6.611 | 23.056 | 0.65 | 23.00 | | | C |
| ANISOU | 1730 | CE | AMET | A | 596 | 3011 | 2312 | 3417 | −34 | −115 | 443 | | C |
| ATOM | 1731 | C | AMET | A | 596 | −18.872 | −10.109 | 26.534 | 0.65 | 8.51 | | | C |
| ANISOU | 1731 | C | AMET | A | 596 | 1216 | 558 | 1458 | 29 | −124 | 181 | | C |
| ATOM | 1732 | O | AMET | A | 596 | −19.519 | −9.803 | 27.542 | 0.65 | 8.11 | | | O |
| ANISOU | 1732 | O | AMET | A | 596 | 1158 | 480 | 1444 | 52 | −108 | 122 | | O |
| ATOM | 1733 | N | BMET | A | 596 | −20.660 | −9.599 | 24.997 | 0.35 | 9.14 | | | N |
| ANISOU | 1733 | N | BMET | A | 596 | 1246 | 647 | 1580 | 69 | −155 | 305 | | N |
| ATOM | 1734 | CA | BMET | A | 596 | −19.213 | −9.531 | 25.133 | 0.35 | 9.35 | | | C |
| ANISOU | 1734 | CA | BMET | A | 596 | 1301 | 658 | 1592 | 34 | −144 | 286 | | C |
| ATOM | 1735 | CB | BMET | A | 596 | −18.719 | −8.108 | 24.906 | 0.35 | 11.27 | | | C |
| ANISOU | 1735 | CB | BMET | A | 596 | 1532 | 841 | 1911 | 33 | −132 | 313 | | C |
| ATOM | 1736 | CG | BMET | A | 596 | −17.231 | −7.946 | 25.151 | 0.35 | 13.58 | | | C |
| ANISOU | 1736 | CG | BMET | A | 596 | 1842 | 1117 | 2202 | −7 | −119 | 282 | | C |
| ATOM | 1737 | SD | BMET | A | 596 | −16.652 | −6.259 | 24.927 | 0.35 | 18.62 | | | S |
| ANISOU | 1737 | SD | BMET | A | 596 | 2460 | 1679 | 2934 | −18 | −102 | 306 | | S |
| ATOM | 1738 | CE | BMET | A | 596 | −16.856 | −6.046 | 23.159 | 0.35 | 24.00 | | | C |
| ANISOU | 1738 | CE | BMET | A | 596 | 3127 | 2406 | 3586 | −19 | −114 | 447 | | C |
| ATOM | 1739 | C | BMET | A | 596 | −18.790 | −10.020 | 26.517 | 0.35 | 8.77 | | | C |
| ANISOU | 1739 | C | BMET | A | 596 | 1249 | 586 | 1496 | 27 | −124 | 182 | | C |
| ATOM | 1740 | O | BMET | A | 596 | −19.302 | −9.524 | 27.528 | 0.35 | 7.81 | | | O |
| ANISOU | 1740 | O | BMET | A | 596 | 1120 | 424 | 1423 | 49 | −108 | 122 | | O |
| ATOM | 1741 | N | PRO | A | 597 | −17.859 | −10.968 | 26.609 | 1.00 | 8.44 | | | N |
| ANISOU | 1741 | N | PRO | A | 597 | 1231 | 594 | 1381 | 0 | −124 | 159 | | N |
| ATOM | 1742 | CA | PRO | A | 597 | −17.387 | −11.410 | 27.929 | 1.00 | 10.40 | | | C |
| ANISOU | 1742 | CA | PRO | A | 597 | 1497 | 855 | 1601 | −3 | −112 | 79 | | C |
| ATOM | 1743 | CB | PRO | A | 597 | −16.273 | −12.411 | 27.605 | 1.00 | 7.95 | | | C |
| ANISOU | 1743 | CB | PRO | A | 597 | 1202 | 593 | 1225 | −27 | −119 | 85 | | C |
| ATOM | 1744 | CG | PRO | A | 597 | −16.496 | −12.809 | 26.142 | 1.00 | 12.74 | | | C |
| ANISOU | 1744 | CG | PRO | A | 597 | 1802 | 1231 | 1805 | −35 | −127 | 152 | | C |
| ATOM | 1745 | CD | PRO | A | 597 | −17.130 | −11.611 | 25.495 | 1.00 | 11.49 | | | C |
| ANISOU | 1745 | CD | PRO | A | 597 | 1625 | 1033 | 1710 | −25 | −133 | 207 | | C |
| ATOM | 1746 | C | PRO | A | 597 | −16.855 | −10.230 | 28.722 | 1.00 | 7.78 | | | C |
| ANISOU | 1746 | C | PRO | A | 597 | 1159 | 460 | 1336 | −7 | −105 | 26 | | C |
| ATOM | 1747 | O | PRO | A | 597 | −16.149 | −9.364 | 28.188 | 1.00 | 8.22 | | | O |
| ANISOU | 1747 | O | PRO | A | 597 | 1205 | 466 | 1451 | −25 | −109 | 52 | | O |
| ATOM | 1748 | N | TYR | A | 598 | −17.181 | −10.207 | 30.014 | 1.00 | 7.42 | | | N |
| ANISOU | 1748 | N | TYR | A | 598 | 1116 | 422 | 1281 | 6 | −92 | −54 | | N |
| ATOM | 1749 | CA | TYR | A | 598 | −16.755 | −9.133 | 30.909 | 1.00 | 8.04 | | | C |
| ANISOU | 1749 | CA | TYR | A | 598 | 1186 | 451 | 1418 | 1 | −85 | −135 | | C |
| ATOM | 1750 | CB | TYR | A | 598 | −15.237 | −9.163 | 31.141 | 1.00 | 9.69 | | | C |
| ANISOU | 1750 | CB | TYR | A | 598 | 1399 | 676 | 1605 | −36 | −102 | −163 | | C |
| ATOM | 1751 | CG | TYR | A | 598 | −14.630 | −10.545 | 31.311 | 1.00 | 8.76 | | | C |
| ANISOU | 1751 | CG | TYR | A | 598 | 1298 | 648 | 1381 | −42 | −116 | −147 | | C |
| ATOM | 1752 | CD1 | TYR | A | 598 | −14.561 | −11.147 | 32.566 | 1.00 | 11.10 | | | C |
| ANISOU | 1752 | CD1 | TYR | A | 598 | 1604 | 1011 | 1602 | −35 | −118 | −203 | | C |
| ATOM | 1753 | CE1 | TYR | A | 598 | −14.012 | −12.419 | 32.720 | 1.00 | 10.86 | | | C |
| ANISOU | 1753 | CE1 | TYR | A | 598 | 1587 | 1049 | 1492 | −34 | −132 | −172 | | C |
| ATOM | 1754 | CZ | TYR | A | 598 | −13.529 | −13.098 | 31.615 | 1.00 | 10.69 | | | C |
| ANISOU | 1754 | CZ | TYR | A | 598 | 1566 | 1026 | 1469 | −41 | −139 | −106 | | C |
| ATOM | 1755 | OH | TYR | A | 598 | −12.979 | −14.352 | 31.776 | 1.00 | 8.50 | | | O |
| ANISOU | 1755 | OH | TYR | A | 598 | 1296 | 800 | 1133 | −33 | −150 | −81 | | O |
| ATOM | 1756 | CE2 | TYR | A | 598 | −13.577 | −12.522 | 30.362 | 1.00 | 6.58 | | | C |
| ANISOU | 1756 | CE2 | TYR | A | 598 | 1037 | 456 | 1007 | −52 | −134 | −63 | | C |
| ATOM | 1757 | CD2 | TYR | A | 598 | −14.132 | −11.248 | 30.218 | 1.00 | 9.40 | | | C |
| ANISOU | 1757 | CD2 | TYR | A | 598 | 1383 | 748 | 1439 | −53 | −125 | −74 | | C |
| ATOM | 1758 | C | TYR | A | 598 | −17.187 | −7.770 | 30.366 | 1.00 | 8.71 | | | C |
| ANISOU | 1758 | C | TYR | A | 598 | 1247 | 433 | 1631 | 12 | −77 | −115 | | C |
| ATOM | 1759 | O | TYR | A | 598 | −16.412 | −6.817 | 30.340 | 1.00 | 9.24 | | | O |
| ANISOU | 1759 | O | TYR | A | 598 | 1304 | 457 | 1750 | −10 | −74 | −130 | | O |
| ATOM | 1760 | N | GLU | A | 599 | −18.449 | −7.677 | 29.927 | 1.00 | 8.81 | | | N |
| ANISOU | 1760 | N | GLU | A | 599 | 1244 | 430 | 1675 | 49 | −70 | −71 | | N |
| ATOM | 1761 | CA | GLU | A | 599 | −18.890 | −6.512 | 29.159 | 1.00 | 9.94 | | | C |
| ANISOU | 1761 | CA | GLU | A | 599 | 1360 | 522 | 1895 | 65 | −65 | −12 | | C |
| ATOM | 1762 | CB | GLU | A | 599 | −20.345 | −6.679 | 28.706 | 1.00 | 13.52 | | | C |
| ANISOU | 1762 | CB | GLU | A | 599 | 1789 | 984 | 2365 | 108 | −67 | 38 | | C |
| ATOM | 1763 | CG | GLU | A | 599 | −21.311 | −6.817 | 29.857 | 1.00 | 13.76 | | | C |
| ANISOU | 1763 | CG | GLU | A | 599 | 1805 | 1011 | 2412 | 141 | −41 | −52 | | C |
| ATOM | 1764 | CD | GLU | A | 599 | −22.767 | −6.931 | 29.400 | 1.00 | 27.23 | | | C |
| ANISOU | 1764 | CD | GLU | A | 599 | 3471 | 2724 | 4151 | 185 | −43 | −3 | | C |
| ATOM | 1765 | OE1 | GLU | A | 599 | −23.016 | −6.913 | 28.174 | 1.00 | 26.85 | | | O |
| ANISOU | 1765 | OE1 | GLU | A | 599 | 3410 | 2689 | 4104 | 189 | −73 | 103 | | O |
| ATOM | 1766 | OE2 | GLU | A | 599 | −23.662 | −7.029 | 30.272 | 1.00 | 28.59 | | | O |
| ANISOU | 1766 | OE2 | GLU | A | 599 | 3621 | 2904 | 4338 | 215 | −12 | −73 | | O |
| ATOM | 1767 | C | GLU | A | 599 | −18.767 | −5.193 | 29.913 | 1.00 | 17.96 | | | C |
| ANISOU | 1767 | C | GLU | A | 599 | 2358 | 1473 | 2992 | 67 | −44 | −87 | | C |
| ATOM | 1768 | O | GLU | A | 599 | −18.747 | −4.134 | 29.273 | 1.00 | 13.10 | | | O |
| ANISOU | 1768 | O | GLU | A | 599 | 1723 | 802 | 2451 | 70 | −43 | −36 | | O |
| ATOM | 1769 | N | ARG | A | 600 | −18.704 | −5.199 | 31.241 | 1.00 | 10.62 | | | N |
| ANISOU | 1769 | N | ARG | A | 600 | 1433 | 553 | 2051 | 66 | −27 | −207 | | N |
| ATOM | 1770 | CA | ARG | A | 600 | −18.569 | −3.925 | 31.932 | 1.00 | 11.68 | | | C |
| ANISOU | 1770 | CA | ARG | A | 600 | 1547 | 631 | 2261 | 65 | −8 | −287 | | C |
| ATOM | 1771 | CB | ARG | A | 600 | −19.401 | −3.932 | 33.216 | 1.00 | 16.49 | | | C |
| ANISOU | 1771 | CB | ARG | A | 600 | 2145 | 1263 | 2857 | 93 | 21 | −405 | | C |
| ATOM | 1772 | CG | ARG | A | 600 | −20.874 | −3.895 | 32.894 | 1.00 | 22.23 | | | C |
| ANISOU | 1772 | CG | ARG | A | 600 | 2844 | 1975 | 3627 | 145 | 38 | −366 | | C |
| ATOM | 1773 | CD | ARG | A | 600 | −21.788 | −4.173 | 34.082 | 1.00 | 30.85 | | | C |
| ANISOU | 1773 | CD | ARG | A | 600 | 3922 | 3111 | 4688 | 173 | 76 | −474 | | C |
| ATOM | 1774 | NE | ARG | A | 600 | −23.172 | −4.225 | 33.612 | 1.00 | 29.27 | | | N |
| ANISOU | 1774 | NE | ARG | A | 600 | 3686 | 2899 | 4536 | 223 | 89 | −421 | | N |
| ATOM | 1775 | CZ | ARG | A | 600 | −23.876 | −3.150 | 33.272 | 1.00 | 32.14 | | | C |
| ANISOU | 1775 | CZ | ARG | A | 600 | 4009 | 3194 | 5008 | 256 | 97 | −400 | | C |
| ATOM | 1776 | NH1 | ARG | A | 600 | −23.333 | −1.939 | 33.364 | 1.00 | 23.12 | | | N |
| ANISOU | 1776 | NH1 | ARG | A | 600 | 2861 | 1981 | 3944 | 243 | 98 | −429 | | N |
| ATOM | 1777 | NH2 | ARG | A | 600 | −25.123 | −3.283 | 32.840 | 1.00 | 38.10 | | | N |
| ANISOU | 1777 | NH2 | ARG | A | 600 | 4724 | 3949 | 5801 | 301 | 102 | −350 | | N |
| ATOM | 1778 | C | ARG | A | 600 | −17.128 | −3.549 | 32.233 | 1.00 | 11.92 | | | C |
| ANISOU | 1778 | C | ARG | A | 600 | 1585 | 654 | 2292 | 15 | −18 | −332 | | C |
| ATOM | 1779 | O | ARG | A | 600 | −16.895 | −2.474 | 32.803 | 1.00 | 12.92 | | | O |
| ANISOU | 1779 | O | ARG | A | 600 | 1692 | 733 | 2486 | 5 | −5 | −405 | | O |
| ATOM | 1780 | N | PHE | A | 601 | −16.165 | −4.389 | 31.840 | 1.00 | 11.15 | | | N |
| ANISOU | 1780 | N | PHE | A | 601 | 1508 | 601 | 2128 | −18 | −41 | −292 | | N |
| ATOM | 1781 | CA | PHE | A | 601 | −14.743 | −4.192 | 32.093 | 1.00 | 11.34 | | | C |
| ANISOU | 1781 | CA | PHE | A | 601 | 1530 | 631 | 2148 | −68 | −55 | −332 | | C |
| ATOM | 1782 | CB | PHE | A | 601 | −14.095 | −5.476 | 32.619 | 1.00 | 15.21 | | | C |
| ANISOU | 1782 | CB | PHE | A | 601 | 2040 | 1206 | 2533 | −85 | −79 | −369 | | C |
| ATOM | 1783 | CG | PHE | A | 601 | −14.521 | −5.866 | 33.998 | 1.00 | 15.77 | | | C |
| ANISOU | 1783 | CG | PHE | A | 601 | 2118 | 1338 | 2536 | −70 | −76 | −482 | | C |
| ATOM | 1784 | CD1 | PHE | A | 601 | −15.114 | −4.943 | 34.855 | 1.00 | 22.34 | | | C |
| ANISOU | 1784 | CD1 | PHE | A | 601 | 2934 | 2151 | 3404 | −55 | −50 | −571 | | C |
| ATOM | 1785 | CE1 | PHE | A | 601 | −15.499 | −5.316 | 36.147 | 1.00 | 29.13 | | | C |
| ANISOU | 1785 | CE1 | PHE | A | 601 | 3797 | 3090 | 4179 | −43 | −40 | −676 | | C |
| ATOM | 1786 | CZ | PHE | A | 601 | −15.276 | −6.613 | 36.586 | 1.00 | 27.11 | | | C |
| ANISOU | 1786 | CZ | PHE | A | 601 | 3564 | 2935 | 3802 | −45 | −60 | −685 | | C |
| ATOM | 1787 | CE2 | PHE | A | 601 | −14.663 | −7.532 | 35.748 | 1.00 | 25.53 | | | C |
| ANISOU | 1787 | CE2 | PHE | A | 601 | 3380 | 2756 | 3565 | −55 | −89 | −578 | | C |
| ATOM | 1788 | CD2 | PHE | A | 601 | −14.291 | −7.156 | 34.457 | 1.00 | 20.50 | | | C |
| ANISOU | 1788 | CD2 | PHE | A | 601 | 2736 | 2029 | 3022 | −69 | −97 | −494 | | C |
| ATOM | 1789 | C | PHE | A | 601 | −14.002 | −3.805 | 30.816 | 1.00 | 11.40 | | | C |
| ANISOU | 1789 | C | PHE | A | 601 | 1530 | 601 | 2201 | −94 | −60 | −226 | | C |
| ATOM | 1790 | O | PHE | A | 601 | −14.383 | −4.211 | 29.717 | 1.00 | 10.93 | | | O |
| ANISOU | 1790 | O | PHE | A | 601 | 1479 | 551 | 2125 | −80 | −63 | −118 | | O |
| ATOM | 1791 | N | THR | A | 602 | −12.912 | −3.056 | 30.979 | 1.00 | 12.07 | | | N |
| ANISOU | 1791 | N | THR | A | 602 | 1597 | 655 | 2335 | −136 | −61 | −261 | | N |
| ATOM | 1792 | CA | THR | A | 602 | −11.923 | −2.915 | 29.912 | 1.00 | 12.62 | | | C |
| ANISOU | 1792 | CA | THR | A | 602 | 1658 | 711 | 2428 | −174 | −64 | −174 | | C |
| ATOM | 1793 | CB | THR | A | 602 | −10.994 | −1.723 | 30.160 | 1.00 | 13.24 | | | C |
| ANISOU | 1793 | CB | THR | A | 602 | 1707 | 728 | 2597 | −216 | −56 | −220 | | C |
| ATOM | 1794 | OG1 | THR | A | 602 | −10.266 | −1.940 | 31.372 | 1.00 | 13.35 | | | O |
| ANISOU | 1794 | OG1 | THR | A | 602 | 1712 | 785 | 2577 | −242 | −73 | −345 | | O |
| ATOM | 1795 | CG2 | THR | A | 602 | −11.760 | −0.395 | 30.247 | 1.00 | 14.38 | | | C |
| ANISOU | 1795 | CG2 | THR | A | 602 | 1835 | 778 | 2850 | −193 | −35 | −230 | | C |
| ATOM | 1796 | C | THR | A | 602 | −11.075 | −4.183 | 29.819 | 1.00 | 11.19 | | | C |
| ANISOU | 1796 | C | THR | A | 602 | 1486 | 608 | 2159 | −197 | −85 | −167 | | C |
| ATOM | 1797 | O | THR | A | 602 | −11.159 | −5.077 | 30.662 | 1.00 | 13.74 | | | O |
| ANISOU | 1797 | O | THR | A | 602 | 1822 | 988 | 2410 | −187 | −102 | −233 | | O |
| ATOM | 1798 | N | ASN | A | 603 | −10.270 | −4.271 | 28.756 | 1.00 | 11.13 | | | N |
| ANISOU | 1798 | N | ASN | A | 603 | 1469 | 604 | 2157 | −227 | −81 | −81 | | N |
| ATOM | 1799 | CA | ASN | A | 603 | −9.295 | −5.352 | 28.652 | 1.00 | 15.07 | | | C |
| ANISOU | 1799 | CA | ASN | A | 603 | 1963 | 1168 | 2596 | −254 | −99 | −81 | | C |
| ATOM | 1800 | CB | ASN | A | 603 | −8.499 | −5.222 | 27.354 | 1.00 | 10.70 | | | C |
| ANISOU | 1800 | CB | ASN | A | 603 | 1390 | 614 | 2061 | −288 | −81 | 21 | | C |
| ATOM | 1801 | CG | ASN | A | 603 | −9.250 | −5.757 | 26.145 | 1.00 | 13.67 | | | C |
| ANISOU | 1801 | CG | ASN | A | 603 | 1787 | 1018 | 2387 | −262 | −72 | 134 | | C |
| ATOM | 1802 | OD1 | ASN | A | 603 | −10.171 | −6.566 | 26.274 | 1.00 | 14.76 | | | O |
| ANISOU | 1802 | OD1 | ASN | A | 603 | 1952 | 1189 | 2467 | −223 | −85 | 133 | | O |
| ATOM | 1803 | ND2 | ASN | A | 603 | −8.840 | −5.323 | 24.963 | 1.00 | 16.21 | | | N |
| ANISOU | 1803 | ND2 | ASN | A | 603 | 2095 | 1339 | 2724 | −285 | −49 | 230 | | N |
| ATOM | 1804 | C | ASN | A | 603 | −8.340 | −5.349 | 29.841 | 1.00 | 14.32 | | | C |
| ANISOU | 1804 | C | ASN | A | 603 | 1845 | 1097 | 2498 | −284 | −122 | −199 | | C |
| ATOM | 1805 | O | ASN | A | 603 | −8.034 | −6.402 | 30.412 | 1.00 | 14.99 | | | O |
| ANISOU | 1805 | O | ASN | A | 603 | 1933 | 1249 | 2515 | −280 | −152 | −244 | | O |
| ATOM | 1806 | N | ASER | A | 604 | −7.832 | −4.170 | 30.204 | 0.38 | 15.59 | | | N |
| ANISOU | 1806 | N | ASER | A | 604 | 1979 | 1210 | 2733 | −314 | −114 | −248 | | N |
| ATOM | 1807 | CA | ASER | A | 604 | −6.917 | −4.073 | 31.338 | 0.38 | 17.07 | | | C |
| ANISOU | 1807 | CA | ASER | A | 604 | 2139 | 1435 | 2913 | −346 | −140 | −365 | | C |
| ATOM | 1808 | CB | ASER | A | 604 | −6.473 | −2.621 | 31.528 | 0.38 | 18.64 | | | C |
| ANISOU | 1808 | CB | ASER | A | 604 | 2309 | 1561 | 3214 | −381 | −124 | −406 | | C |
| ATOM | 1809 | OG | ASER | A | 604 | −5.638 | −2.493 | 32.666 | 0.38 | 18.73 | | | O |
| ANISOU | 1809 | OG | ASER | A | 604 | 2289 | 1618 | 3209 | −412 | −154 | −526 | | O |
| ATOM | 1810 | C | ASER | A | 604 | −7.565 | −4.606 | 32.610 | 0.38 | 15.57 | | | C |
| ANISOU | 1810 | C | ASER | A | 604 | 1969 | 1303 | 2644 | −313 | −163 | −461 | | C |
| ATOM | 1811 | O | ASER | A | 604 | −6.960 | −5.390 | 33.350 | 0.38 | 14.13 | | | O |
| ANISOU | 1811 | O | ASER | A | 604 | 1775 | 1208 | 2386 | −320 | −200 | −521 | | O |
| ATOM | 1812 | N | BSER | A | 604 | −7.853 | −4.173 | 30.226 | 0.62 | 15.63 | | | N |
| ANISOU | 1812 | N | BSER | A | 604 | 1986 | 1216 | 2739 | −314 | −114 | −249 | | N |
| ATOM | 1813 | CA | BSER | A | 604 | −6.910 | −4.102 | 31.338 | 0.62 | 17.34 | | | C |
| ANISOU | 1813 | CA | BSER | A | 604 | 2172 | 1470 | 2945 | −346 | −141 | −365 | | C |
| ATOM | 1814 | CB | BSER | A | 604 | −6.378 | −2.681 | 31.472 | 0.62 | 19.14 | | | C |
| ANISOU | 1814 | CB | BSER | A | 604 | 2370 | 1628 | 3275 | −384 | −125 | −401 | | C |
| ATOM | 1815 | OG | BSER | A | 604 | −5.504 | −2.412 | 30.400 | 0.62 | 13.81 | | | O |
| ANISOU | 1815 | OG | BSER | A | 604 | 1670 | 926 | 2653 | −424 | −109 | −317 | | O |
| ATOM | 1816 | C | BSER | A | 604 | −7.547 | −4.568 | 32.641 | 0.62 | 15.60 | | | C |
| ANISOU | 1816 | C | BSER | A | 604 | 1972 | 1307 | 2650 | −314 | −163 | −465 | | C |
| ATOM | 1817 | O | BSER | A | 604 | −6.907 | −5.266 | 33.438 | 0.62 | 12.02 | | | O |
| ANISOU | 1817 | O | BSER | A | 604 | 1504 | 938 | 2123 | −323 | −200 | −531 | | O |
| ATOM | 1818 | N | GLU | A | 605 | −8.804 | −4.187 | 32.878 | 1.00 | 12.18 | | | N |
| ANISOU | 1818 | N | GLU | A | 605 | 1564 | 836 | 2227 | −274 | −141 | −474 | | N |
| ATOM | 1819 | CA | GLU | A | 605 | −9.498 | −4.632 | 34.081 | 1.00 | 12.10 | | | C |
| ANISOU | 1819 | CA | GLU | A | 605 | 1572 | 888 | 2139 | −244 | −151 | −564 | | C |
| ATOM | 1820 | CB | GLU | A | 605 | −10.790 | −3.829 | 34.264 | 1.00 | 13.76 | | | C |
| ANISOU | 1820 | CB | GLU | A | 605 | 1793 | 1036 | 2399 | −208 | −116 | −581 | | C |
| ATOM | 1821 | CG | GLU | A | 605 | −10.544 | −2.358 | 34.610 | 1.00 | 20.01 | | | C |
| ANISOU | 1821 | CG | GLU | A | 605 | 2555 | 1757 | 3292 | −229 | −101 | −646 | | C |
| ATOM | 1822 | CD | GLU | A | 605 | −11.783 | −1.502 | 34.414 | 1.00 | 28.69 | | | C |
| ANISOU | 1822 | CD | GLU | A | 605 | 3655 | 2772 | 4472 | −190 | −65 | −631 | | C |
| ATOM | 1823 | OE1 | GLU | A | 605 | −12.698 | −1.939 | 33.687 | 1.00 | 22.45 | | | O |
| ANISOU | 1823 | OE1 | GLU | A | 605 | 2885 | 1972 | 3675 | −152 | −55 | −541 | | O |
| ATOM | 1824 | OE2 | GLU | A | 605 | −11.843 | −0.393 | 34.985 | 1.00 | 30.38 | | | O |
| ANISOU | 1824 | OE2 | GLU | A | 605 | 3846 | 2935 | 4764 | −196 | −50 | −712 | | O |
| ATOM | 1825 | C | GLU | A | 605 | −9.798 | −6.123 | 34.020 | 1.00 | 16.63 | | | C |
| ANISOU | 1825 | C | GLU | A | 605 | 2173 | 1535 | 2613 | −217 | −172 | −528 | | C |
| ATOM | 1826 | O | GLU | A | 605 | −9.747 | −6.814 | 35.049 | 1.00 | 11.88 | | | O |
| ANISOU | 1826 | O | GLU | A | 605 | 1575 | 1023 | 1914 | −208 | −198 | −598 | | O |
| ATOM | 1827 | N | THR | A | 606 | −10.118 | −6.632 | 32.822 | 1.00 | 11.01 | | | N |
| ANISOU | 1827 | N | THR | A | 606 | 1477 | 791 | 1915 | −203 | −162 | −415 | | N |
| ATOM | 1828 | CA | THR | A | 606 | −10.357 | −8.063 | 32.649 | 1.00 | 9.35 | | | C |
| ANISOU | 1828 | CA | THR | A | 606 | 1292 | 666 | 1594 | −171 | −172 | −353 | | C |
| ATOM | 1829 | CB | THR | A | 606 | −10.788 | −8.346 | 31.203 | 1.00 | 14.23 | | | C |
| ANISOU | 1829 | CB | THR | A | 606 | 1922 | 1251 | 2231 | −160 | −153 | −231 | | C |
| ATOM | 1830 | OG1 | THR | A | 606 | −12.010 | −7.657 | 30.925 | 1.00 | 14.25 | | | O |
| ANISOU | 1830 | OG1 | THR | A | 606 | 1935 | 1182 | 2299 | −137 | −131 | −213 | | O |
| ATOM | 1831 | CG2 | THR | A | 606 | −10.989 | −9.843 | 30.962 | 1.00 | 7.97 | | | C |
| ANISOU | 1831 | CG2 | THR | A | 606 | 1153 | 544 | 1333 | −131 | −158 | −176 | | C |
| ATOM | 1832 | C | THR | A | 606 | −9.113 | −8.862 | 33.009 | 1.00 | 10.14 | | | C |
| ANISOU | 1832 | C | THR | A | 606 | 1375 | 856 | 1622 | −186 | −205 | −364 | | C |
| ATOM | 1833 | O | THR | A | 606 | −9.183 | −9.853 | 33.747 | 1.00 | 12.39 | | | O |
| ANISOU | 1833 | O | THR | A | 606 | 1673 | 1231 | 1805 | −160 | −222 | −371 | | O |
| ATOM | 1834 | N | ALA | A | 607 | −7.962 | −8.439 | 32.494 | 1.00 | 9.57 | | | N |
| ANISOU | 1834 | N | ALA | A | 607 | 1266 | 761 | 1608 | −228 | −213 | −357 | | N |
| ATOM | 1835 | CA | ALA | A | 607 | −6.710 | −9.104 | 32.837 | 1.00 | 11.26 | | | C |
| ANISOU | 1835 | CA | ALA | A | 607 | 1449 | 1060 | 1771 | −240 | −247 | −372 | | C |
| ATOM | 1836 | CB | ALA | A | 607 | −5.549 | −8.392 | 32.146 | 1.00 | 11.75 | | | C |
| ANISOU | 1836 | CB | ALA | A | 607 | 1461 | 1079 | 1925 | −295 | −243 | −365 | | C |
| ATOM | 1837 | C | ALA | A | 607 | −6.497 | −9.157 | 34.353 | 1.00 | 10.19 | | | C |
| ANISOU | 1837 | C | ALA | A | 607 | 1302 | 1002 | 1569 | −238 | −286 | −474 | | C |
| ATOM | 1838 | O | ALA | A | 607 | −6.151 | −10.206 | 34.904 | 1.00 | 12.27 | | | O |
| ANISOU | 1838 | O | ALA | A | 607 | 1564 | 1363 | 1735 | −211 | −316 | −459 | | O |
| ATOM | 1839 | N . | AGLU | A | 608 | −6.708 | −8.034 | 35.040 | 0.56 | 14.10 | | | N |
| ANISOU | 1839 | N | AGLU | A | 608 | 1787 | 1457 | 2115 | −265 | −286 | −578 | | N |
| ATOM | 1840 | CA | AGLU | A | 608 | −6.527 | −8.015 | 36.492 | 0.56 | 17.37 | | | C |
| ANISOU | 1840 | CA | AGLU | A | 608 | 2187 | 1964 | 2449 | −267 | −323 | −691 | | C |
| ATOM | 1841 | CB | AGLU | A | 608 | −6.649 | −6.584 | 37.012 | 0.56 | 19.64 | | | C |
| ANISOU | 1841 | CB | AGLU | A | 608 | 2463 | 2196 | 2804 | −295 | −297 | −776 | | C |
| ATOM | 1842 | CG | AGLU | A | 608 | −6.686 | −6.454 | 38.533 | 0.56 | 23.44 | | | C |
| ANISOU | 1842 | CG | AGLU | A | 608 | 2938 | 2780 | 3188 | −291 | −317 | −881 | | C |
| ATOM | 1843 | CD | AGLU | A | 608 | −5.350 | −6.749 | 39.188 | 0.56 | 27.73 | | | C |
| ANISOU | 1843 | CD | AGLU | A | 608 | 3436 | 3432 | 3668 | −317 | −376 | −916 | | C |
| ATOM | 1844 | OE1 | AGLU | A | 608 | −4.324 | −6.778 | 38.475 | 0.56 | 29.01 | | | O |
| ANISOU | 1844 | OE1 | AGLU | A | 608 | 3561 | 3571 | 3890 | −346 | −394 | −878 | | O |
| ATOM | 1845 | OE2 | AGLU | A | 608 | −5.325 | −6.953 | 40.422 | 0.56 | 27.26 | | | O |
| ANISOU | 1845 | OE2 | AGLU | A | 608 | 3373 | 3488 | 3497 | −308 | −402 | −976 | | O |
| ATOM | 1846 | C | AGLU | A | 608 | −7.529 | −8.931 | 37.189 | 0.56 | 19.99 | | | C |
| ANISOU | 1846 | C | AGLU | A | 608 | 2564 | 2377 | 2656 | −213 | −317 | −668 | | C |
| ATOM | 1847 | O | AGLU | A | 608 | −7.173 | −9.643 | 38.137 | 0.56 | 19.57 | | | O |
| ANISOU | 1847 | O | AGLU | A | 608 | 2503 | 2445 | 2486 | −199 | −355 | −684 | | O |
| ATOM | 1848 | N | BGLU | A | 608 | −6.699 | −8.031 | 35.040 | 0.44 | 14.40 | | | N |
| ANISOU | 1848 | N | BGLU | A | 608 | 1825 | 1495 | 2153 | −265 | −286 | −579 | | N |
| ATOM | 1849 | CA | BGLU | A | 608 | −6.527 | −8.019 | 36.492 | 0.44 | 17.44 | | | C |
| ANISOU | 1849 | CA | BGLU | A | 608 | 2197 | 1973 | 2458 | −267 | −323 | −690 | | C |
| ATOM | 1850 | CB | BGLU | A | 608 | −6.674 | −6.597 | 37.031 | 0.44 | 19.61 | | | C |
| ANISOU | 1850 | CB | BGLU | A | 608 | 2459 | 2193 | 2797 | −294 | −297 | −777 | | C |
| ATOM | 1851 | CG | BGLU | A | 608 | −5.652 | −5.592 | 36.505 | 0.44 | 19.11 | | | C |
| ANISOU | 1851 | CG | BGLU | A | 608 | 2353 | 2059 | 2847 | −346 | −291 | −780 | | C |
| ATOM | 1852 | CD | BGLU | A | 608 | −4.289 | −5.712 | 37.173 | 0.44 | 26.84 | | | C |
| ANISOU | 1852 | CD | BGLU | A | 608 | 3280 | 3132 | 3785 | −380 | −342 | −836 | | C |
| ATOM | 1853 | OE1 | BGLU | A | 608 | −4.134 | −6.558 | 38.082 | 0.44 | 29.47 | | | O |
| ANISOU | 1853 | OE1 | BGLU | A | 608 | 3611 | 3591 | 3995 | −359 | −386 | −865 | | O |
| ATOM | 1854 | OE2 | BGLU | A | 608 | −3.371 | −4.952 | 36.792 | 0.44 | 24.48 | | | O |
| ANISOU | 1854 | OE2 | BGLU | A | 608 | 2941 | 2785 | 3576 | −426 | −338 | −843 | | O |
| ATOM | 1855 | C | BGLU | A | 608 | −7.524 | −8.951 | 37.173 | 0.44 | 19.65 | | | C |
| ANISOU | 1855 | C | BGLU | A | 608 | 2520 | 2333 | 2612 | −213 | −317 | −666 | | C |
| ATOM | 1856 | O | BGLU | A | 608 | −7.162 | −9.693 | 38.095 | 0.44 | 19.70 | | | O |
| ANISOU | 1856 | O | BGLU | A | 608 | 2520 | 2461 | 2502 | −197 | −355 | −677 | | O |
| ATOM | 1857 | N | HIS | A | 609 | −8.787 | −8.921 | 36.732 | 1.00 | 15.26 | | | N |
| ANISOU | 1857 | N | HIS | A | 609 | 2004 | 1714 | 2080 | −184 | −271 | −624 | | N |
| ATOM | 1858 | CA | HIS | A | 609 | −9.830 | −9.797 | 37.285 | 1.00 | 15.66 | | | C |
| ANISOU | 1858 | CA | HIS | A | 609 | 2092 | 1830 | 2026 | −139 | −255 | −592 | | C |
| ATOM | 1859 | CB | HIS | A | 609 | −11.125 | −9.544 | 36.489 | 1.00 | 17.80 | | | C |
| ANISOU | 1859 | CB | HIS | A | 609 | 2390 | 2008 | 2365 | −117 | −205 | −545 | | C |
| ATOM | 1860 | CG | HIS | A | 609 | −12.358 | −10.286 | 36.948 | 1.00 | 20.94 | | | C |
| ANISOU | 1860 | CG | HIS | A | 609 | 2817 | 2455 | 2684 | −78 | −178 | −518 | | C |
| ATOM | 1861 | ND1 | HIS | A | 609 | −13.178 | −9.821 | 37.957 | 1.00 | 19.06 | | | N |
| ANISOU | 1861 | ND1 | HIS | A | 609 | 2580 | 2247 | 2415 | −67 | −153 | −606 | | N |
| ATOM | 1862 | CE1 | HIS | A | 609 | −14.215 | −10.632 | 38.097 | 1.00 | 13.67 | | | C |
| ANISOU | 1862 | CE1 | HIS | A | 609 | 1919 | 1604 | 1673 | −37 | −124 | −550 | | C |
| ATOM | 1863 | NE2 | HIS | A | 609 | −14.123 | −11.583 | 37.184 | 1.00 | 19.13 | | | N |
| ANISOU | 1863 | NE2 | HIS | A | 609 | 2624 | 2279 | 2364 | −29 | −131 | −435 | | N |
| ATOM | 1864 | CD2 | HIS | A | 609 | −12.986 | −11.377 | 36.437 | 1.00 | 18.74 | | | C |
| ANISOU | 1864 | CD2 | HIS | A | 609 | 2563 | 2190 | 2369 | −51 | −163 | −415 | | C |
| ATOM | 1865 | C | HIS | A | 609 | −9.410 | −11.256 | 37.192 | 1.00 | 16.15 | | | C |
| ANISOU | 1865 | C | HIS | A | 609 | 2163 | 1971 | 2000 | −113 | −278 | −491 | | C |
| ATOM | 1866 | O | HIS | A | 609 | −9.445 | −12.002 | 38.180 | 1.00 | 15.33 | | | O |
| ANISOU | 1866 | O | HIS | A | 609 | 2067 | 1972 | 1786 | −92 | −296 | −488 | | O |
| ATOM | 1867 | N | ILE | A | 610 | −8.996 | −11.666 | 35.994 | 1.00 | 15.69 | | | N |
| ANISOU | 1867 | N | ILE | A | 610 | 2104 | 1864 | 1996 | −113 | −274 | −407 | | N |
| ATOM | 1868 | CA | ILE | A | 610 | −8.596 | −13.042 | 35.725 | 1.00 | 13.46 | | | C |
| ANISOU | 1868 | CA | ILE | A | 610 | 1826 | 1628 | 1660 | −85 | −287 | −317 | | C |
| ATOM | 1869 | CB | ILE | A | 610 | −8.368 | −13.209 | 34.206 | 1.00 | 19.01 | | | C |
| ANISOU | 1869 | CB | ILE | A | 610 | 2526 | 2260 | 2436 | −91 | −266 | −249 | | C |
| ATOM | 1870 | CG1 | ILE | A | 610 | −9.707 | −13.181 | 33.487 | 1.00 | 22.34 | | | C |
| ANISOU | 1870 | CG1 | ILE | A | 610 | 2984 | 2623 | 2883 | −79 | −225 | −210 | | C |
| ATOM | 1871 | CD1 | ILE | A | 610 | −10.678 | −14.168 | 34.029 | 1.00 | 26.98 | | | C |
| ANISOU | 1871 | CD1 | ILE | A | 610 | 3602 | 3248 | 3401 | −46 | −213 | −179 | | C |
| ATOM | 1872 | CG2 | ILE | A | 610 | −7.564 | −14.457 | 33.888 | 1.00 | 26.58 | | | C |
| ANISOU | 1872 | CG2 | ILE | A | 610 | 3472 | 3260 | 3367 | −69 | −282 | −187 | | C |
| ATOM | 1873 | C | ILE | A | 610 | −7.354 | −13.390 | 36.523 | 1.00 | 18.43 | | | C |
| ANISOU | 1873 | C | ILE | A | 610 | 2418 | 2349 | 2237 | −87 | −340 | −338 | | C |
| ATOM | 1874 | O | ILE | A | 610 | −7.250 | −14.478 | 37.106 | 1.00 | 16.92 | | | O |
| ANISOU | 1874 | O | ILE | A | 610 | 2232 | 2231 | 1965 | −53 | −361 | −287 | | O |
| ATOM | 1875 | N | ALA | A | 611 | −6.409 | −12.452 | 36.588 | 1.00 | 18.53 | | | N |
| ANISOU | 1875 | N | ALA | A | 611 | 2387 | 2357 | 2299 | −128 | −365 | −410 | | N |
| ATOM | 1876 | CA | ALA | A | 611 | −5.161 | −12.685 | 37.306 | 1.00 | 20.04 | | | C |
| ANISOU | 1876 | CA | ALA | A | 611 | 2527 | 2641 | 2445 | −134 | −424 | −438 | | C |
| ATOM | 1877 | CB | ALA | A | 611 | −4.212 | −11.501 | 37.118 | 1.00 | 19.28 | | | C |
| ANISOU | 1877 | CB | ALA | A | 611 | 2377 | 2513 | 2437 | −193 | −440 | −523 | | C |
| ATOM | 1878 | C | ALA | A | 611 | −5.405 | −12.928 | 38.784 | 1.00 | 26.62 | | | C |
| ANISOU | 1878 | C | ALA | A | 611 | 3367 | 3592 | 3156 | −117 | −458 | −478 | | C |
| ATOM | 1879 | O | ALA | A | 611 | −4.663 | −13.695 | 39.411 | 1.00 | 33.29 | | | O |
| ANISOU | 1879 | O | ALA | A | 611 | 4183 | 4538 | 3927 | −94 | −509 | −445 | | O |
| ATOM | 1880 | N | GLN | A | 612 | −6.437 | −12.292 | 39.358 | 1.00 | 20.67 | | | N |
| ANISOU | 1880 | N | GLN | A | 612 | 2645 | 2834 | 2376 | −124 | −430 | −546 | | N |
| ATOM | 1881 | CA | GLN | A | 612 | −6.800 | −12.468 | 40.760 | 1.00 | 21.63 | | | C |
| ANISOU | 1881 | CA | GLN | A | 612 | 2774 | 3080 | 2364 | −111 | −450 | −590 | | C |
| ATOM | 1882 | CB | GLN | A | 612 | −7.521 | −11.234 | 41.308 | 1.00 | 30.57 | | | C |
| ANISOU | 1882 | CB | GLN | A | 612 | 3913 | 4194 | 3510 | −141 | −421 | −728 | | C |
| ATOM | 1883 | CG | GLN | A | 612 | −6.772 | −9.933 | 41.245 | 1.00 | 44.59 | | | C |
| ANISOU | 1883 | CG | GLN | A | 612 | 5646 | 5909 | 5385 | −193 | −429 | −832 | | C |
| ATOM | 1884 | CD | GLN | A | 612 | −7.677 | −8.751 | 41.590 | 1.00 | 53.38 | | | C |
| ANISOU | 1884 | CD | GLN | A | 612 | 6776 | 6955 | 6552 | −206 | −373 | −923 | | C |
| ATOM | 1885 | OE1 | GLN | A | 612 | −8.841 | −8.931 | 41.972 | 1.00 | 50.83 | | | O |
| ANISOU | 1885 | OE1 | GLN | A | 612 | 6487 | 6650 | 6176 | −178 | −334 | −927 | | O |
| ATOM | 1886 | NE2 | GLN | A | 612 | −7.149 | −7.537 | 41.446 | 1.00 | 55.21 | | | N |
| ANISOU | 1886 | NE2 | GLN | A | 612 | 6976 | 7106 | 6894 | −247 | −366 | −991 | | N |
| ATOM | 1887 | C | GLN | A | 612 | −7.706 | −13.666 | 40.991 | 1.00 | 26.92 | | | C |
| ANISOU | 1887 | C | GLN | A | 612 | 3490 | 3788 | 2951 | −63 | −424 | −479 | | C |
| ATOM | 1888 | O | GLN | A | 612 | −8.271 | −13.780 | 42.086 | 1.00 | 24.86 | | | O |
| ANISOU | 1888 | O | GLN | A | 612 | 3244 | 3626 | 2577 | −53 | −421 | −503 | | O |
| ATOM | 1889 | N | GLY | A | 613 | −7.896 | −14.519 | 39.990 | 1.00 | 23.00 | | | N |
| ANISOU | 1889 | N | GLY | A | 613 | 3014 | 3216 | 2508 | −37 | −399 | −367 | | N |
| ATOM | 1890 | CA | GLY | A | 613 | −8.681 | −15.720 | 40.158 | 1.00 | 18.90 | | | C |
| ANISOU | 1890 | CA | GLY | A | 613 | 2531 | 2717 | 1931 | 2 | −375 | −260 | | C |
| ATOM | 1891 | C | GLY | A | 613 | −10.173 | −15.561 | 40.004 | 1.00 | 19.47 | | | C |
| ANISOU | 1891 | C | GLY | A | 613 | 2644 | 2738 | 2015 | 3 | −310 | −263 | | C |
| ATOM | 1892 | O | GLY | A | 613 | −10.907 | −16.515 | 40.287 | 1.00 | 19.83 | | | O |
| ANISOU | 1892 | O | GLY | A | 613 | 2717 | 2809 | 2010 | 27 | −285 | −182 | | O |
| ATOM | 1893 | N | LEU | A | 614 | −10.655 | −14.405 | 39.564 | 1.00 | 17.23 | | | N |
| ANISOU | 1893 | N | LEU | A | 614 | 2361 | 2380 | 1805 | −21 | −281 | −346 | | N |
| ATOM | 1894 | CA | LEU | A | 614 | −12.087 | −14.230 | 39.372 | 1.00 | 12.05 | | | C |
| ANISOU | 1894 | CA | LEU | A | 614 | 1732 | 1675 | 1171 | −14 | −223 | −347 | | C |
| ATOM | 1895 | CB | LEU | A | 614 | −12.503 | −12.793 | 39.693 | 1.00 | 14.48 | | | C |
| ANISOU | 1895 | CB | LEU | A | 614 | 2027 | 1955 | 1518 | −34 | −203 | −475 | | C |
| ATOM | 1896 | CG | LEU | A | 614 | −12.052 | −12.336 | 41.090 | 1.00 | 19.11 | | | C |
| ANISOU | 1896 | CG | LEU | A | 614 | 2596 | 2659 | 2007 | −47 | −229 | −582 | | C |
| ATOM | 1897 | CD1 | LEU | A | 614 | −12.396 | −10.859 | 41.341 | 1.00 | 22.09 | | | C |
| ANISOU | 1897 | CD1 | LEU | A | 614 | 2956 | 2988 | 2450 | −69 | −205 | −731 | | C |
| ATOM | 1898 | CD2 | LEU | A | 614 | −12.661 | −13.217 | 42.179 | 1.00 | 17.08 | | | C |
| ANISOU | 1898 | CD2 | LEU | A | 614 | 2355 | 2530 | 1604 | −25 | −213 | −541 | | C |
| ATOM | 1899 | C | LEU | A | 614 | −12.467 | −14.603 | 37.941 | 1.00 | 11.66 | | | C |
| ANISOU | 1899 | C | LEU | A | 614 | 1696 | 1519 | 1216 | −7 | −200 | −270 | | C |
| ATOM | 1900 | O | LEU | A | 614 | −11.628 | −14.644 | 37.046 | 1.00 | 12.28 | | | O |
| ANISOU | 1900 | O | LEU | A | 614 | 1762 | 1551 | 1354 | −16 | −221 | −244 | | O |
| ATOM | 1901 | N | ARG | A | 615 | −13.751 | −14.886 | 37.740 | 1.00 | 9.37 | | | N |
| ANISOU | 1901 | N | ARG | A | 615 | 1425 | 1202 | 934 | 5 | −155 | −238 | | N |
| ATOM | 1902 | CA | ARG | A | 615 | −14.194 | −15.539 | 36.523 | 1.00 | 10.71 | | | C |
| ANISOU | 1902 | CA | ARG | A | 615 | 1606 | 1302 | 1162 | 11 | −139 | −161 | | C |
| ATOM | 1903 | CB | ARG | A | 615 | −14.393 | −17.046 | 36.756 | 1.00 | 9.70 | | | C |
| ANISOU | 1903 | CB | ARG | A | 615 | 1493 | 1209 | 983 | 27 | −132 | −73 | | C |
| ATOM | 1904 | CG | ARG | A | 615 | −13.130 | −17.782 | 37.212 | 1.00 | 11.46 | | | C |
| ANISOU | 1904 | CG | ARG | A | 615 | 1710 | 1482 | 1161 | 38 | −174 | −35 | | C |
| ATOM | 1905 | CD | ARG | A | 615 | −12.053 | −17.765 | 36.136 | 1.00 | 14.73 | | | C |
| ANISOU | 1905 | CD | ARG | A | 615 | 2108 | 1846 | 1644 | 34 | −200 | −30 | | C |
| ATOM | 1906 | NE | ARG | A | 615 | −10.889 | −18.597 | 36.453 | 1.00 | 16.09 | | | N |
| ANISOU | 1906 | NE | ARG | A | 615 | 2264 | 2059 | 1792 | 53 | −237 | 14 | | N |
| ATOM | 1907 | CZ | ARG | A | 615 | −9.697 | −18.105 | 36.747 | 1.00 | 14.56 | | | C |
| ANISOU | 1907 | CZ | ARG | A | 615 | 2038 | 1904 | 1589 | 48 | −280 | −24 | | C |
| ATOM | 1908 | NH1 | ARG | A | 615 | −9.538 | −16.796 | 36.773 | 1.00 | 9.35 | | | N |
| ANISOU | 1908 | NH1 | ARG | A | 615 | 1364 | 1240 | 947 | 18 | −286 | −110 | | N |
| ATOM | 1909 | NH2 | ARG | A | 615 | −8.672 | −18.913 | 37.003 | 1.00 | 14.74 | | | N |
| ANISOU | 1909 | NH2 | ARG | A | 615 | 2037 | 1965 | 1598 | 73 | −317 | 23 | | N |
| ATOM | 1910 | C | ARG | A | 615 | −15.486 | −14.901 | 36.028 | 1.00 | 10.14 | | | C |
| ANISOU | 1910 | C | ARG | A | 615 | 1532 | 1172 | 1147 | 12 | −100 | −180 | | C |
| ATOM | 1911 | O | ARG | A | 615 | −16.132 | −14.128 | 36.734 | 1.00 | 14.01 | | | O |
| ANISOU | 1911 | O | ARG | A | 615 | 2015 | 1676 | 1630 | 15 | −78 | −246 | | O |
| ATOM | 1912 | N | LEU | A | 616 | −15.841 | −15.225 | 34.782 | 1.00 | 10.09 | | | N |
| ANISOU | 1912 | N | LEU | A | 616 | 1529 | 1107 | 1198 | 12 | −94 | −125 | | N |
| ATOM | 1913 | CA | LEU | A | 616 | −17.204 | −15.036 | 34.298 | 1.00 | 9.71 | | | C |
| ANISOU | 1913 | CA | LEU | A | 616 | 1475 | 1024 | 1191 | 19 | −63 | −115 | | C |
| ATOM | 1914 | CB | LEU | A | 616 | −17.324 | −15.522 | 32.844 | 1.00 | 6.34 | | | C |
| ANISOU | 1914 | CB | LEU | A | 616 | 1048 | 555 | 807 | 13 | −71 | −55 | | C |
| ATOM | 1915 | CG | LEU | A | 616 | −16.487 | −14.797 | 31.800 | 1.00 | 6.19 | | | C |
| ANISOU | 1915 | CG | LEU | A | 616 | 1021 | 493 | 838 | 2 | −93 | −50 | | C |
| ATOM | 1916 | CD1 | LEU | A | 616 | −16.393 | −15.647 | 30.512 | 1.00 | 5.78 | | | C |
| ANISOU | 1916 | CD1 | LEU | A | 616 | 972 | 437 | 789 | −7 | −100 | 4 | | C |
| ATOM | 1917 | CD2 | LEU | A | 616 | −17.098 | −13.447 | 31.514 | 1.00 | 6.48 | | | C |
| ANISOU | 1917 | CD2 | LEU | A | 616 | 1041 | 479 | 943 | 7 | −86 | −71 | | C |
| ATOM | 1918 | C | LEU | A | 616 | −18.169 | −15.829 | 35.172 | 1.00 | 11.66 | | | C |
| ANISOU | 1918 | C | LEU | A | 616 | 1727 | 1324 | 1381 | 27 | −30 | −100 | | C |
| ATOM | 1919 | O | LEU | A | 616 | −17.792 | −16.811 | 35.828 | 1.00 | 12.91 | | | O |
| ANISOU | 1919 | O | LEU | A | 616 | 1898 | 1532 | 1475 | 27 | −33 | −64 | | O |
| ATOM | 1920 | N | TYR | A | 617 | −19.438 | −15.421 | 35.176 | 1.00 | 9.27 | | | N |
| ANISOU | 1920 | N | TYR | A | 617 | 1405 | 1008 | 1107 | 36 | 3 | −118 | | N |
| ATOM | 1921 | CA | TYR | A | 617 | −20.410 | −16.189 | 35.944 | 1.00 | 9.18 | | | C |
| ANISOU | 1921 | CA | TYR | A | 617 | 1391 | 1049 | 1048 | 37 | 43 | −98 | | C |
| ATOM | 1922 | CB | TYR | A | 617 | −20.901 | −15.414 | 37.174 | 1.00 | 10.28 | | | C |
| ANISOU | 1922 | CB | TYR | A | 617 | 1516 | 1240 | 1148 | 48 | 78 | −173 | | C |
| ATOM | 1923 | CG | TYR | A | 617 | −21.640 | −14.117 | 36.942 | 1.00 | 10.33 | | | C |
| ANISOU | 1923 | CG | TYR | A | 617 | 1492 | 1199 | 1235 | 66 | 96 | −243 | | C |
| ATOM | 1924 | CD1 | TYR | A | 617 | −20.950 | −12.904 | 36.803 | 1.00 | 12.02 | | | C |
| ANISOU | 1924 | CD1 | TYR | A | 617 | 1703 | 1362 | 1501 | 70 | 73 | −310 | | C |
| ATOM | 1925 | CE1 | TYR | A | 617 | −21.641 | −11.711 | 36.627 | 1.00 | 15.69 | | | C |
| ANISOU | 1925 | CE1 | TYR | A | 617 | 2136 | 1765 | 2058 | 92 | 92 | −369 | | C |
| ATOM | 1926 | CZ | TYR | A | 617 | −23.035 | −11.719 | 36.622 | 1.00 | 15.79 | | | C |
| ANISOU | 1926 | CZ | TYR | A | 617 | 2115 | 1780 | 2103 | 114 | 134 | −366 | | C |
| ATOM | 1927 | OH | TYR | A | 617 | −23.751 | −10.547 | 36.456 | 1.00 | 15.64 | | | O |
| ANISOU | 1927 | OH | TYR | A | 617 | 2058 | 1696 | 2191 | 146 | 153 | −420 | | O |
| ATOM | 1928 | CE2 | TYR | A | 617 | −23.730 | −12.904 | 36.781 | 1.00 | 15.14 | | | C |
| ANISOU | 1928 | CE2 | TYR | A | 617 | 2031 | 1760 | 1960 | 105 | 157 | −307 | | C |
| ATOM | 1929 | CD2 | TYR | A | 617 | −23.029 | −14.092 | 36.941 | 1.00 | 9.45 | | | C |
| ANISOU | 1929 | CD2 | TYR | A | 617 | 1347 | 1089 | 1155 | 79 | 140 | −245 | | C |
| ATOM | 1930 | C | TYR | A | 617 | −21.562 | −16.653 | 35.044 | 1.00 | 7.85 | | | C |
| ANISOU | 1930 | C | TYR | A | 617 | 1202 | 846 | 934 | 33 | 60 | −55 | | C |
| ATOM | 1931 | O | TYR | A | 617 | −21.577 | −16.425 | 33.835 | 1.00 | 12.04 | | | O |
| ANISOU | 1931 | O | TYR | A | 617 | 1724 | 1325 | 1524 | 32 | 35 | −40 | | O |
| ATOM | 1932 | N | ARG | A | 618 | −22.497 | −17.355 | 35.658 | 1.00 | 8.43 | | | N |
| ANISOU | 1932 | N | ARG | A | 618 | 1264 | 959 | 980 | 27 | 101 | −33 | | N |
| ATOM | 1933 | CA | ARG | A | 618 | −23.430 | −18.222 | 34.930 | 1.00 | 7.55 | | | C |
| ANISOU | 1933 | CA | ARG | A | 618 | 1132 | 826 | 909 | 11 | 114 | 15 | | C |
| ATOM | 1934 | CB | ARG | A | 618 | −24.170 | −19.088 | 35.949 | 1.00 | 8.71 | | | C |
| ANISOU | 1934 | CB | ARG | A | 618 | 1272 | 1027 | 1012 | −4 | 166 | 47 | | C |
| ATOM | 1935 | CG | ARG | A | 618 | −25.148 | −20.086 | 35.350 | 1.00 | 9.89 | | | C |
| ANISOU | 1935 | CG | ARG | A | 618 | 1395 | 1153 | 1211 | −32 | 185 | 92 | | C |
| ATOM | 1936 | CD | ARG | A | 618 | −25.769 | −20.908 | 36.456 | 1.00 | 10.74 | | | C |
| ANISOU | 1936 | CD | ARG | A | 618 | 1494 | 1309 | 1276 | −52 | 244 | 136 | | C |
| ATOM | 1937 | NE | ARG | A | 618 | −26.995 | −21.544 | 35.999 | 1.00 | 16.67 | | | N |
| ANISOU | 1937 | NE | ARG | A | 618 | 2200 | 2044 | 2091 | −82 | 273 | 157 | | N |
| ATOM | 1938 | CZ | ARG | A | 618 | −27.889 | −22.078 | 36.814 | 1.00 | 15.03 | | | C |
| ANISOU | 1938 | CZ | ARG | A | 618 | 1965 | 1877 | 1869 | −105 | 337 | 190 | | C |
| ATOM | 1939 | NH1 | ARG | A | 618 | −27.698 | −22.045 | 38.134 | 1.00 | 13.74 | | | N |
| ANISOU | 1939 | NH1 | ARG | A | 618 | 1819 | 1786 | 1616 | −98 | 378 | 210 | | N |
| ATOM | 1940 | NH2 | ARG | A | 618 | −28.977 | −22.632 | 36.314 | 1.00 | 14.41 | | | N |
| ANISOU | 1940 | NH2 | ARG | A | 618 | 1836 | 1778 | 1860 | −139 | 359 | 202 | | N |
| ATOM | 1941 | C | ARG | A | 618 | −24.422 | −17.425 | 34.085 | 1.00 | 7.54 | | | C |
| ANISOU | 1941 | C | ARG | A | 618 | 1089 | 794 | 982 | 22 | 112 | −9 | | C |
| ATOM | 1942 | O | ARG | A | 618 | −25.162 | −16.602 | 34.635 | 1.00 | 8.91 | | | O |
| ANISOU | 1942 | O | ARG | A | 618 | 1232 | 983 | 1170 | 42 | 142 | −54 | | O |
| ATOM | 1943 | N | PRO | A | 619 | −24.469 | −17.628 | 32.764 | 1.00 | 7.15 | | | N |
| ANISOU | 1943 | N | PRO | A | 619 | 1031 | 708 | 978 | 13 | 76 | 17 | | N |
| ATOM | 1944 | CA | PRO | A | 619 | −25.525 | −17.007 | 31.948 | 1.00 | 7.37 | | | C |
| ANISOU | 1944 | CA | PRO | A | 619 | 1010 | 722 | 1070 | 25 | 68 | 13 | | C |
| ATOM | 1945 | CB | PRO | A | 619 | −25.186 | −17.449 | 30.517 | 1.00 | 6.92 | | | C |
| ANISOU | 1945 | CB | PRO | A | 619 | 957 | 648 | 1026 | 7 | 21 | 45 | | C |
| ATOM | 1946 | CG | PRO | A | 619 | −23.706 | −17.809 | 30.564 | 1.00 | 8.47 | | | C |
| ANISOU | 1946 | CG | PRO | A | 619 | 1206 | 831 | 1180 | −2 | 5 | 51 | | C |
| ATOM | 1947 | CD | PRO | A | 619 | −23.530 | −18.417 | 31.948 | 1.00 | 6.68 | | | C |
| ANISOU | 1947 | CD | PRO | A | 619 | 1002 | 628 | 910 | −6 | 42 | 48 | | C |
| ATOM | 1948 | C | PRO | A | 619 | −26.899 | −17.516 | 32.345 | 1.00 | 7.79 | | | C |
| ANISOU | 1948 | C | PRO | A | 619 | 1015 | 806 | 1137 | 15 | 109 | 14 | | C |
| ATOM | 1949 | O | PRO | A | 619 | −27.068 | −18.670 | 32.730 | 1.00 | 8.47 | | | O |
| ANISOU | 1949 | O | PRO | A | 619 | 1109 | 912 | 1199 | −16 | 134 | 39 | | O |
| ATOM | 1950 | N | HIS | A | 620 | −27.905 | −16.658 | 32.176 | 1.00 | 8.20 | | | N |
| ANISOU | 1950 | N | HIS | A | 620 | 1012 | 858 | 1244 | 42 | 115 | −6 | | N |
| ATOM | 1951 | CA | HIS | A | 620 | −29.257 | −17.050 | 32.557 | 1.00 | 10.22 | | | C |
| ANISOU | 1951 | CA | HIS | A | 620 | 1210 | 1151 | 1524 | 34 | 158 | −9 | | C |
| ATOM | 1952 | CB | HIS | A | 620 | −30.201 | −15.886 | 32.304 | 1.00 | 11.02 | | | C |
| ANISOU | 1952 | CB | HIS | A | 620 | 1244 | 1243 | 1699 | 79 | 157 | −34 | | C |
| ATOM | 1953 | CG | HIS | A | 620 | −29.953 | −14.717 | 33.212 | 1.00 | 20.41 | | | C |
| ANISOU | 1953 | CG | HIS | A | 620 | 2441 | 2417 | 2897 | 122 | 188 | −92 | | C |
| ATOM | 1954 | ND1 | HIS | A | 620 | −30.371 | −13.437 | 32.918 | 1.00 | 25.62 | | | N |
| ANISOU | 1954 | ND1 | HIS | A | 620 | 3059 | 3035 | 3643 | 174 | 178 | −117 | | N |
| ATOM | 1955 | CE1 | HIS | A | 620 | −30.023 | −12.624 | 33.900 | 1.00 | 25.46 | | | C |
| ANISOU | 1955 | CE1 | HIS | A | 620 | 3053 | 3001 | 3621 | 199 | 215 | −187 | | C |
| ATOM | 1956 | NE2 | HIS | A | 620 | −29.397 | −13.331 | 34.823 | 1.00 | 23.31 | | | N |
| ANISOU | 1956 | NE2 | HIS | A | 620 | 2830 | 2777 | 3248 | 165 | 244 | −204 | | N |
| ATOM | 1957 | CD2 | HIS | A | 620 | −29.340 | −14.642 | 34.417 | 1.00 | 22.42 | | | C |
| ANISOU | 1957 | CD2 | HIS | A | 620 | 2736 | 2694 | 3088 | 120 | 228 | −136 | | C |
| ATOM | 1958 | C | HIS | A | 620 | −29.727 | −18.318 | 31.838 | 1.00 | 13.92 | | | C |
| ANISOU | 1958 | C | HIS | A | 620 | 1659 | 1629 | 2002 | −14 | 145 | 28 | | C |
| ATOM | 1959 | O | HIS | A | 620 | −30.494 | −19.099 | 32.413 | 1.00 | 10.88 | | | O |
| ANISOU | 1959 | O | HIS | A | 620 | 1245 | 1272 | 1619 | −43 | 192 | 35 | | O |
| ATOM | 1960 | N | LEU | A | 621 | −29.272 | −18.567 | 30.612 | 1.00 | 11.21 | | | N |
| ANISOU | 1960 | N | LEU | A | 621 | 1329 | 1266 | 1665 | −27 | 88 | 46 | | N |
| ATOM | 1961 | CA | LEU | A | 621 | −29.762 | −19.731 | 29.873 | 1.00 | 10.47 | | | C |
| ANISOU | 1961 | CA | LEU | A | 621 | 1210 | 1181 | 1587 | −76 | 74 | 58 | | C |
| ATOM | 1962 | CB | LEU | A | 621 | −29.771 | −19.445 | 28.360 | 1.00 | 8.65 | | | C |
| ANISOU | 1962 | CB | LEU | A | 621 | 959 | 961 | 1369 | −74 | 5 | 61 | | C |
| ATOM | 1963 | CG | LEU | A | 621 | −30.843 | −18.456 | 27.884 | 1.00 | 8.84 | | | C |
| ANISOU | 1963 | CG | LEU | A | 621 | 908 | 1013 | 1439 | −40 | −21 | 66 | | C |
| ATOM | 1964 | CD1 | LEU | A | 621 | −30.627 | −18.054 | 26.439 | 1.00 | 10.07 | | | C |
| ANISOU | 1964 | CD1 | LEU | A | 621 | 1054 | 1189 | 1584 | −32 | −94 | 90 | | C |
| ATOM | 1965 | CD2 | LEU | A | 621 | −32.224 | −19.090 | 28.048 | 1.00 | 13.18 | | | C |
| ANISOU | 1965 | CD2 | LEU | A | 621 | 1379 | 1598 | 2031 | −69 | 2 | 53 | | C |
| ATOM | 1966 | C | LEU | A | 621 | −28.973 | −21.009 | 30.142 | 1.00 | 10.00 | | | C |
| ANISOU | 1966 | C | LEU | A | 621 | 1204 | 1097 | 1499 | −116 | 90 | 71 | | C |
| ATOM | 1967 | O | LEU | A | 621 | −29.363 | −22.069 | 29.645 | 1.00 | 10.15 | | | O |
| ANISOU | 1967 | O | LEU | A | 621 | 1203 | 1110 | 1545 | −161 | 87 | 70 | | O |
| ATOM | 1968 | N | ALA | A | 622 | −27.894 | −20.953 | 30.922 | 1.00 | 7.86 | | | N |
| ANISOU | 1968 | N | ALA | A | 622 | 994 | 811 | 1183 | −99 | 104 | 82 | | N |
| ATOM | 1969 | CA | ALA | A | 622 | −27.078 | −22.128 | 31.197 | 1.00 | 9.01 | | | C |
| ANISOU | 1969 | CA | ALA | A | 622 | 1185 | 928 | 1310 | −125 | 115 | 108 | | C |
| ATOM | 1970 | CB | ALA | A | 622 | −25.604 | −21.736 | 31.356 | 1.00 | 7.27 | | | C |
| ANISOU | 1970 | CB | ALA | A | 622 | 1023 | 695 | 1044 | −96 | 91 | 110 | | C |
| ATOM | 1971 | C | ALA | A | 622 | −27.592 | −22.840 | 32.447 | 1.00 | 10.06 | | | C |
| ANISOU | 1971 | C | ALA | A | 622 | 1313 | 1073 | 1436 | −145 | 178 | 142 | | C |
| ATOM | 1972 | O | ALA | A | 622 | −27.714 | −22.230 | 33.519 | 1.00 | 12.95 | | | O |
| ANISOU | 1972 | O | ALA | A | 622 | 1681 | 1479 | 1759 | −122 | 212 | 144 | | O |
| ATOM | 1973 | N | SER | A | 623 | −27.908 | −24.126 | 32.303 | 1.00 | 12.08 | | | N |
| ANISOU | 1973 | N | SER | A | 623 | 1559 | 1296 | 1735 | −190 | 196 | 168 | | N |
| ATOM | 1974 | CA | SER | A | 623 | −28.210 | −24.952 | 33.456 | 1.00 | 12.15 | | | C |
| ANISOU | 1974 | CA | SER | A | 623 | 1571 | 1307 | 1739 | −214 | 258 | 226 | | C |
| ATOM | 1975 | CB | SER | A | 623 | −28.670 | −26.338 | 33.022 | 1.00 | 12.19 | | | C |
| ANISOU | 1975 | CB | SER | A | 623 | 1554 | 1251 | 1825 | −272 | 274 | 246 | | C |
| ATOM | 1976 | OG | SER | A | 623 | −27.584 | −27.043 | 32.433 | 1.00 | 9.54 | | | O |
| ANISOU | 1976 | OG | SER | A | 623 | 1262 | 851 | 1512 | −272 | 241 | 246 | | O |
| ATOM | 1977 | C | SER | A | 623 | −26.974 | −25.075 | 34.343 | 1.00 | 12.89 | | | C |
| ANISOU | 1977 | C | SER | A | 623 | 1728 | 1403 | 1767 | −186 | 260 | 272 | | C |
| ATOM | 1978 | O | SER | A | 623 | −25.846 | −24.714 | 33.956 | 1.00 | 8.62 | | | O |
| ANISOU | 1978 | O | SER | A | 623 | 1225 | 848 | 1203 | −156 | 213 | 253 | | O |
| ATOM | 1979 | N | GLU | A | 624 | −27.188 | −25.611 | 35.550 | 1.00 | 10.91 | | | N |
| ANISOU | 1979 | N | GLU | A | 624 | 1482 | 1179 | 1485 | −197 | 314 | 339 | | N |
| ATOM | 1980 | CA | GLU | A | 624 | −26.050 | −25.855 | 36.434 | 1.00 | 12.17 | | | C |
| ANISOU | 1980 | CA | GLU | A | 624 | 1695 | 1353 | 1577 | −171 | 310 | 397 | | C |
| ATOM | 1981 | CB | GLU | A | 624 | −26.522 | −26.406 | 37.784 | 1.00 | 15.37 | | | C |
| ANISOU | 1981 | CB | GLU | A | 624 | 2096 | 1811 | 1933 | −189 | 377 | 485 | | C |
| ATOM | 1982 | CG | GLU | A | 624 | −27.340 | −25.400 | 38.595 | 1.00 | 27.95 | | | C |
| ANISOU | 1982 | CG | GLU | A | 624 | 3659 | 3507 | 3452 | −180 | 421 | 446 | | C |
| ATOM | 1983 | CD | GLU | A | 624 | −27.561 | −25.844 | 40.035 | 1.00 | 44.84 | | | C |
| ANISOU | 1983 | CD | GLU | A | 624 | 5802 | 5729 | 5505 | −192 | 486 | 534 | | C |
| ATOM | 1984 | OE1 | GLU | A | 624 | −26.640 | −26.445 | 40.631 | 1.00 | 51.47 | | | O |
| ANISOU | 1984 | OE1 | GLU | A | 624 | 6686 | 6577 | 6295 | −180 | 473 | 617 | | O |
| ATOM | 1985 | OE2 | GLU | A | 624 | −28.658 | −25.591 | 40.568 | 1.00 | 49.83 | | | O |
| ANISOU | 1985 | OE2 | GLU | A | 624 | 6390 | 6426 | 6118 | −210 | 551 | 524 | | O |
| ATOM | 1986 | C | GLU | A | 624 | −25.047 | −26.807 | 35.796 | 1.00 | 9.97 | | | C |
| ANISOU | 1986 | C | GLU | A | 624 | 1447 | 989 | 1352 | −172 | 274 | 425 | | C |
| ATOM | 1987 | O | GLU | A | 624 | −23.836 | −26.608 | 35.922 | 1.00 | 12.36 | | | O |
| ANISOU | 1987 | O | GLU | A | 624 | 1787 | 1296 | 1613 | −135 | 235 | 431 | | O |
| ATOM | 1988 | N | LYS | A | 625 | −25.531 | −27.864 | 35.120 | 1.00 | 12.45 | | | N |
| ANISOU | 1988 | N | LYS | A | 625 | 1741 | 1223 | 1766 | −214 | 287 | 435 | | N |
| ATOM | 1989 | CA | LYS | A | 625 | −24.611 | −28.808 | 34.484 | 1.00 | 11.42 | | | C |
| ANISOU | 1989 | CA | LYS | A | 625 | 1634 | 1001 | 1702 | −212 | 260 | 445 | | C |
| ATOM | 1990 | CB | LYS | A | 625 | −25.356 | −30.032 | 33.951 | 1.00 | 11.56 | | | C |
| ANISOU | 1990 | CB | LYS | A | 625 | 1623 | 929 | 1842 | −268 | 289 | 450 | | C |
| ATOM | 1991 | CG | LYS | A | 625 | −25.950 | −30.925 | 35.032 | 1.00 | 20.48 | | | C |
| ANISOU | 1991 | CG | LYS | A | 625 | 2744 | 2055 | 2983 | −295 | 339 | 541 | | C |
| ATOM | 1992 | CD | LYS | A | 625 | −26.182 | −32.343 | 34.505 | 1.00 | 26.68 | | | C |
| ANISOU | 1992 | CD | LYS | A | 625 | 3514 | 2748 | 3875 | −331 | 337 | 524 | | C |
| ATOM | 1993 | CE | LYS | A | 625 | −26.753 | −33.272 | 35.579 | 1.00 | 35.97 | | | C |
| ANISOU | 1993 | CE | LYS | A | 625 | 4683 | 3917 | 5066 | −358 | 384 | 619 | | C |
| ATOM | 1994 | NZ | LYS | A | 625 | −28.219 | −33.072 | 35.772 | 1.00 | 40.62 | | | N |
| ANISOU | 1994 | NZ | LYS | A | 625 | 5220 | 4545 | 5669 | −413 | 434 | 617 | | N |
| ATOM | 1995 | C | LYS | A | 625 | −23.821 | −28.147 | 33.362 | 1.00 | 9.22 | | | C |
| ANISOU | 1995 | C | LYS | A | 625 | 1367 | 719 | 1415 | −185 | 200 | 359 | | C |
| ATOM | 1996 | O | LYS | A | 625 | −22.622 | −28.415 | 33.201 | 1.00 | 9.62 | | | O |
| ANISOU | 1996 | O | LYS | A | 625 | 1448 | 739 | 1469 | −155 | 172 | 366 | | O |
| ATOM | 1997 | N | VAL | A | 626 | −24.462 | −27.271 | 32.584 | 1.00 | 8.76 | | | N |
| ANISOU | 1997 | N | VAL | A | 626 | 1281 | 698 | 1349 | −193 | 181 | 284 | | N |
| ATOM | 1998 | CA | VAL | A | 626 | −23.756 | −26.587 | 31.504 | 1.00 | 8.07 | | | C |
| ANISOU | 1998 | CA | VAL | A | 626 | 1202 | 617 | 1245 | −171 | 130 | 218 | | C |
| ATOM | 1999 | CB | VAL | A | 626 | −24.755 | −25.854 | 30.589 | 1.00 | 11.17 | | | C |
| ANISOU | 1999 | CB | VAL | A | 626 | 1553 | 1046 | 1645 | −189 | 112 | 158 | | C |
| ATOM | 2000 | CG1 | VAL | A | 626 | −24.011 | −24.851 | 29.696 | 1.00 | 13.30 | | | C |
| ANISOU | 2000 | CG1 | VAL | A | 626 | 1834 | 1342 | 1877 | −160 | 63 | 116 | | C |
| ATOM | 2001 | CG2 | VAL | A | 626 | −25.543 | −26.873 | 29.756 | 1.00 | 13.37 | | | C |
| ANISOU | 2001 | CG2 | VAL | A | 626 | 1794 | 1284 | 2000 | −241 | 117 | 125 | | C |
| ATOM | 2002 | C | VAL | A | 626 | −22.709 | −25.635 | 32.060 | 1.00 | 13.99 | | | C |
| ANISOU | 2002 | C | VAL | A | 626 | 1985 | 1410 | 1920 | −124 | 107 | 227 | | C |
| ATOM | 2003 | O | VAL | A | 626 | −21.580 | −25.562 | 31.550 | 1.00 | 11.69 | | | O |
| ANISOU | 2003 | O | VAL | A | 626 | 1713 | 1104 | 1623 | −103 | 75 | 209 | | O |
| ATOM | 2004 | N | TYR | A | 627 | −23.067 | −24.882 | 33.107 | 1.00 | 9.02 | | | N |
| ANISOU | 2004 | N | TYR | A | 627 | 1355 | 839 | 1234 | −109 | 126 | 245 | | N |
| ATOM | 2005 | CA | TYR | A | 627 | −22.113 | −23.989 | 33.757 | 1.00 | 8.03 | | | C |
| ANISOU | 2005 | CA | TYR | A | 627 | 1255 | 758 | 1038 | −72 | 105 | 241 | | C |
| ATOM | 2006 | CB | TYR | A | 627 | −22.818 | −23.218 | 34.889 | 1.00 | 8.17 | | | C |
| ANISOU | 2006 | CB | TYR | A | 627 | 1262 | 844 | 998 | −65 | 138 | 238 | | C |
| ATOM | 2007 | CG | TYR | A | 627 | −21.980 | −22.124 | 35.551 | 1.00 | 12.15 | | | C |
| ANISOU | 2007 | CG | TYR | A | 627 | 1785 | 1400 | 1434 | −33 | 116 | 204 | | C |
| ATOM | 2008 | CD1 | TYR | A | 627 | −21.253 | −21.209 | 34.780 | 1.00 | 10.34 | | | C |
| ANISOU | 2008 | CD1 | TYR | A | 627 | 1560 | 1150 | 1220 | −18 | 73 | 152 | | C |
| ATOM | 2009 | CE1 | TYR | A | 627 | −20.500 | −20.201 | 35.379 | 1.00 | 9.20 | | | C |
| ANISOU | 2009 | CE1 | TYR | A | 627 | 1424 | 1041 | 1028 | 2 | 55 | 112 | | C |
| ATOM | 2010 | CZ | TYR | A | 627 | −20.477 | −20.092 | 36.774 | 1.00 | 10.25 | | | C |
| ANISOU | 2010 | CZ | TYR | A | 627 | 1564 | 1247 | 1082 | 9 | 75 | 112 | | C |
| ATOM | 2011 | OH | TYR | A | 627 | −19.740 | −19.093 | 37.368 | 1.00 | 11.95 | | | O |
| ANISOU | 2011 | OH | TYR | A | 627 | 1784 | 1503 | 1253 | 23 | 54 | 53 | | O |
| ATOM | 2012 | CE2 | TYR | A | 627 | −21.198 | −20.976 | 37.562 | 1.00 | 16.02 | | | C |
| ANISOU | 2012 | CE2 | TYR | A | 627 | 2293 | 2015 | 1778 | −2 | 118 | 170 | | C |
| ATOM | 2013 | CD2 | TYR | A | 627 | −21.956 | −21.984 | 36.943 | 1.00 | 14.24 | | | C |
| ANISOU | 2013 | CD2 | TYR | A | 627 | 2057 | 1738 | 1616 | −24 | 141 | 221 | | C |
| ATOM | 2014 | C | TYR | A | 627 | −20.906 | −24.753 | 34.298 | 1.00 | 7.71 | | | C |
| ANISOU | 2014 | C | TYR | A | 627 | 1245 | 703 | 980 | −54 | 93 | 294 | | C |
| ATOM | 2015 | O | TYR | A | 627 | −19.773 | −24.269 | 34.221 | 1.00 | 10.86 | | | O |
| ANISOU | 2015 | O | TYR | A | 627 | 1659 | 1115 | 1353 | −28 | 57 | 275 | | O |
| ATOM | 2016 | N | THR | A | 628 | −21.125 | −25.936 | 34.868 | 1.00 | 8.34 | | | N |
| ANISOU | 2016 | N | THR | A | 628 | 1328 | 757 | 1083 | −66 | 124 | 367 | | N |
| ATOM | 2017 | CA | THR | A | 628 | −20.001 | −26.718 | 35.374 | 1.00 | 12.52 | | | C |
| ANISOU | 2017 | CA | THR | A | 628 | 1880 | 1268 | 1609 | −41 | 108 | 433 | | C |
| ATOM | 2018 | CB | THR | A | 628 | −20.502 | −28.001 | 36.031 | 1.00 | 13.97 | | | C |
| ANISOU | 2018 | CB | THR | A | 628 | 2063 | 1413 | 1831 | −59 | 151 | 532 | | C |
| ATOM | 2019 | OG1 | THR | A | 628 | −21.188 | −27.673 | 37.241 | 1.00 | 11.51 | | | O |
| ANISOU | 2019 | OG1 | THR | A | 628 | 1750 | 1187 | 1436 | −66 | 187 | 579 | | O |
| ATOM | 2020 | CG2 | THR | A | 628 | −19.342 | −28.937 | 36.337 | 1.00 | 15.28 | | | C |
| ANISOU | 2020 | CG2 | THR | A | 628 | 2246 | 1535 | 2025 | −25 | 131 | 610 | | C |
| ATOM | 2021 | C | THR | A | 628 | −19.007 | −27.035 | 34.258 | 1.00 | 11.52 | | | C |
| ANISOU | 2021 | C | THR | A | 628 | 1756 | 1077 | 1543 | −27 | 73 | 395 | | C |
| ATOM | 2022 | O | THR | A | 628 | −17.788 | −26.989 | 34.465 | 1.00 | 8.31 | | | O |
| ANISOU | 2022 | O | THR | A | 628 | 1359 | 682 | 1115 | 8 | 41 | 409 | | O |
| ATOM | 2023 | N | ILE | A | 629 | −19.512 | −27.317 | 33.060 | 1.00 | 8.59 | | | N |
| ANISOU | 2023 | N | ILE | A | 629 | 1370 | 651 | 1242 | −54 | 78 | 339 | | N |
| ATOM | 2024 | CA | ILE | A | 629 | −18.631 | −27.634 | 31.942 | 1.00 | 7.84 | | | C |
| ANISOU | 2024 | CA | ILE | A | 629 | 1274 | 509 | 1197 | −45 | 54 | 290 | | C |
| ATOM | 2025 | CB | ILE | A | 629 | −19.444 | −28.195 | 30.764 | 1.00 | 7.92 | | | C |
| ANISOU | 2025 | CB | ILE | A | 629 | 1262 | 469 | 1277 | −86 | 68 | 230 | | C |
| ATOM | 2026 | CG1 | ILE | A | 629 | −20.065 | −29.533 | 31.156 | 1.00 | 15.89 | | | C |
| ANISOU | 2026 | CG1 | ILE | A | 629 | 2266 | 1401 | 2371 | −112 | 107 | 278 | | C |
| ATOM | 2027 | CD1 | ILE | A | 629 | −20.962 | −30.105 | 30.091 | 1.00 | 18.42 | | | C |
| ANISOU | 2027 | CD1 | ILE | A | 629 | 2558 | 1677 | 2764 | −163 | 119 | 205 | | C |
| ATOM | 2028 | CG2 | ILE | A | 629 | −18.531 | −28.363 | 29.523 | 1.00 | 8.93 | | | C |
| ANISOU | 2028 | CG2 | ILE | A | 629 | 1387 | 573 | 1434 | −77 | 47 | 160 | | C |
| ATOM | 2029 | C | ILE | A | 629 | −17.815 | −26.413 | 31.531 | 1.00 | 9.28 | | | C |
| ANISOU | 2029 | C | ILE | A | 629 | 1459 | 746 | 1321 | −23 | 17 | 241 | | C |
| ATOM | 2030 | O | ILE | A | 629 | −16.592 | −26.494 | 31.374 | 1.00 | 9.77 | | | O |
| ANISOU | 2030 | O | ILE | A | 629 | 1523 | 802 | 1387 | 4 | −5 | 237 | | O |
| ATOM | 2031 | N | MET | A | 630 | −18.476 | −25.270 | 31.310 | 1.00 | 9.68 | | | N |
| ANISOU | 2031 | N | MET | A | 630 | 1502 | 845 | 1331 | −36 | 12 | 203 | | N |
| ATOM | 2032 | CA | MET | A | 630 | −17.716 | −24.112 | 30.852 | 1.00 | 9.58 | | | C |
| ANISOU | 2032 | CA | MET | A | 630 | 1489 | 867 | 1282 | −22 | −19 | 164 | | C |
| ATOM | 2033 | CB | MET | A | 630 | −18.652 | −22.984 | 30.394 | 1.00 | 13.54 | | | C |
| ANISOU | 2033 | CB | MET | A | 630 | 1978 | 1398 | 1770 | −37 | −21 | 130 | | C |
| ATOM | 2034 | CG | MET | A | 630 | −19.294 | −22.155 | 31.497 | 1.00 | 11.76 | | | C |
| ANISOU | 2034 | CG | MET | A | 630 | 1752 | 1210 | 1508 | −29 | −8 | 138 | | C |
| ATOM | 2035 | SD | MET | A | 630 | −20.357 | −20.783 | 30.915 | 1.00 | 11.81 | | | S |
| ANISOU | 2035 | SD | MET | A | 630 | 1732 | 1232 | 1525 | −33 | −12 | 101 | | S |
| ATOM | 2036 | CE | MET | A | 630 | −21.562 | −21.629 | 29.871 | 1.00 | 8.94 | | | C |
| ANISOU | 2036 | CE | MET | A | 630 | 1339 | 853 | 1205 | −63 | −5 | 99 | | C |
| ATOM | 2037 | C | MET | A | 630 | −16.755 | −23.642 | 31.939 | 1.00 | 10.21 | | | C |
| ANISOU | 2037 | C | MET | A | 630 | 1581 | 985 | 1314 | 7 | −36 | 188 | | C |
| ATOM | 2038 | O | MET | A | 630 | −15.606 | −23.302 | 31.648 | 1.00 | 8.52 | | | O |
| ANISOU | 2038 | O | MET | A | 630 | 1364 | 777 | 1095 | 21 | −61 | 170 | | O |
| ATOM | 2039 | N | TYR | A | 631 | −17.181 | −23.686 | 33.205 | 1.00 | 6.75 | | | N |
| ANISOU | 2039 | N | TYR | A | 631 | 1151 | 580 | 836 | 12 | −22 | 227 | | N |
| ATOM | 2040 | CA | TYR | A | 631 | −16.301 | −23.265 | 34.302 | 1.00 | 7.02 | | | C |
| ANISOU | 2040 | CA | TYR | A | 631 | 1191 | 669 | 806 | 36 | −44 | 242 | | C |
| ATOM | 2041 | CB | TYR | A | 631 | −17.088 | −23.263 | 35.618 | 1.00 | 10.15 | | | C |
| ANISOU | 2041 | CB | TYR | A | 631 | 1594 | 1123 | 1140 | 34 | −18 | 277 | | C |
| ATOM | 2042 | CG | TYR | A | 631 | −16.479 | −22.401 | 36.690 | 1.00 | 9.91 | | | C |
| ANISOU | 2042 | CG | TYR | A | 631 | 1565 | 1176 | 1024 | 48 | −40 | 256 | | C |
| ATOM | 2043 | CD2 | TYR | A | 631 | −15.559 | −22.932 | 37.587 | 1.00 | 13.11 | | | C |
| ANISOU | 2043 | CD2 | TYR | A | 631 | 1973 | 1630 | 1376 | 71 | −66 | 312 | | C |
| ATOM | 2044 | CE2 | TYR | A | 631 | −14.983 | −22.153 | 38.573 | 1.00 | 14.31 | | | C |
| ANISOU | 2044 | CE2 | TYR | A | 631 | 2120 | 1876 | 1439 | 80 | −94 | 280 | | C |
| ATOM | 2045 | CZ | TYR | A | 631 | −15.314 | −20.814 | 38.673 | 1.00 | 15.03 | | | C |
| ANISOU | 2045 | CZ | TYR | A | 631 | 2206 | 1997 | 1509 | 65 | −90 | 182 | | C |
| ATOM | 2046 | OH | TYR | A | 631 | −14.728 | −20.054 | 39.673 | 1.00 | 19.83 | | | O |
| ANISOU | 2046 | OH | TYR | A | 631 | 2806 | 2698 | 2031 | 68 | −117 | 130 | | O |
| ATOM | 2047 | CE1 | TYR | A | 631 | −16.221 | −20.249 | 37.799 | 1.00 | 9.51 | | | C |
| ANISOU | 2047 | CE1 | TYR | A | 631 | 1505 | 1234 | 875 | 48 | −60 | 133 | | C |
| ATOM | 2048 | CD1 | TYR | A | 631 | −16.802 | −21.047 | 36.795 | 1.00 | 10.53 | | | C |
| ANISOU | 2048 | CD1 | TYR | A | 631 | 1636 | 1286 | 1079 | 41 | −39 | 175 | | C |
| ATOM | 2049 | C | TYR | A | 631 | −15.066 | −24.155 | 34.428 | 1.00 | 9.42 | | | C |
| ANISOU | 2049 | C | TYR | A | 631 | 1494 | 958 | 1126 | 62 | −67 | 287 | | C |
| ATOM | 2050 | O | TYR | A | 631 | −13.997 | −23.683 | 34.849 | 1.00 | 11.11 | | | O |
| ANISOU | 2050 | O | TYR | A | 631 | 1701 | 1216 | 1304 | 82 | −103 | 279 | | O |
| ATOM | 2051 | N | SER | A | 632 | −15.193 | −25.449 | 34.111 | 1.00 | 7.60 | | | N |
| ANISOU | 2051 | N | SER | A | 632 | 1265 | 662 | 959 | 65 | −49 | 332 | | N |
| ATOM | 2052 | CA | SER | A | 632 | −14.051 | −26.352 | 34.191 | 1.00 | 8.33 | | | C |
| ANISOU | 2052 | CA | SER | A | 632 | 1349 | 725 | 1090 | 99 | −69 | 377 | | C |
| ATOM | 2053 | CB | SER | A | 632 | −14.486 | −27.797 | 33.904 | 1.00 | 12.72 | | | C |
| ANISOU | 2053 | CB | SER | A | 632 | 1907 | 1187 | 1738 | 96 | −37 | 423 | | C |
| ATOM | 2054 | OG | SER | A | 632 | −14.743 | −27.996 | 32.519 | 1.00 | 12.23 | | | O |
| ANISOU | 2054 | OG | SER | A | 632 | 1837 | 1066 | 1745 | 73 | −21 | 346 | | O |
| ATOM | 2055 | C | SER | A | 632 | −12.931 | −25.959 | 33.234 | 1.00 | 8.70 | | | C |
| ANISOU | 2055 | C | SER | A | 632 | 1378 | 764 | 1164 | 111 | −95 | 315 | | C |
| ATOM | 2056 | O | SER | A | 632 | −11.778 | −26.365 | 33.443 | 1.00 | 9.77 | | | O |
| ANISOU | 2056 | O | SER | A | 632 | 1495 | 900 | 1316 | 147 | −120 | 341 | | O |
| ATOM | 2057 | N | CYS | A | 633 | −13.236 | −25.186 | 32.187 | 1.00 | 7.65 | | | N |
| ANISOU | 2057 | N | CYS | A | 633 | 1243 | 630 | 1035 | 83 | −88 | 242 | | N |
| ATOM | 2058 | CA | CYS | A | 633 | −12.202 | −24.705 | 31.270 | 1.00 | 9.27 | | | C |
| ANISOU | 2058 | CA | CYS | A | 633 | 1427 | 841 | 1253 | 88 | −104 | 190 | | C |
| ATOM | 2059 | CB | CYS | A | 633 | −12.850 | −24.190 | 29.980 | 1.00 | 9.52 | | | C |
| ANISOU | 2059 | CB | CYS | A | 633 | 1459 | 864 | 1294 | 53 | −88 | 133 | | C |
| ATOM | 2060 | SG | CYS | A | 633 | −13.826 | −25.465 | 29.100 | 1.00 | 10.05 | | | S |
| ANISOU | 2060 | SG | CYS | A | 633 | 1529 | 867 | 1421 | 34 | −54 | 116 | | S |
| ATOM | 2061 | C | CYS | A | 633 | −11.334 | −23.604 | 31.861 | 1.00 | 13.52 | | | C |
| ANISOU | 2061 | C | CYS | A | 633 | 1953 | 1442 | 1741 | 95 | −138 | 179 | | C |
| ATOM | 2062 | O | CYS | A | 633 | −10.351 | −23.202 | 31.216 | 1.00 | 9.56 | | | O |
| ANISOU | 2062 | O | CYS | A | 633 | 1428 | 949 | 1255 | 96 | −150 | 145 | | O |
| ATOM | 2063 | N | TRP | A | 634 | −11.683 | −23.082 | 33.040 | 1.00 | 10.41 | | | N |
| ANISOU | 2063 | N | TRP | A | 634 | 1571 | 1096 | 1287 | 94 | −150 | 197 | | N |
| ATOM | 2064 | CA | TRP | A | 634 | −11.053 | −21.874 | 33.567 | 1.00 | 7.99 | | | C |
| ANISOU | 2064 | CA | TRP | A | 634 | 1252 | 849 | 936 | 89 | −181 | 160 | | C |
| ATOM | 2065 | CB | TRP | A | 634 | −12.098 | −20.798 | 33.853 | 1.00 | 7.14 | | | C |
| ANISOU | 2065 | CB | TRP | A | 634 | 1160 | 757 | 796 | 63 | −166 | 122 | | C |
| ATOM | 2066 | CG | TRP | A | 634 | −12.943 | −20.450 | 32.624 | 1.00 | 7.60 | | | C |
| ANISOU | 2066 | CG | TRP | A | 634 | 1224 | 764 | 898 | 41 | −139 | 98 | | C |
| ATOM | 2067 | CD1 | TRP | A | 634 | −12.551 | −20.468 | 31.311 | 1.00 | 7.89 | | | C |
| ANISOU | 2067 | CD1 | TRP | A | 634 | 1251 | 770 | 979 | 31 | −136 | 85 | | C |
| ATOM | 2068 | NE1 | TRP | A | 634 | −13.616 | −20.103 | 30.490 | 1.00 | 7.58 | | | N |
| ANISOU | 2068 | NE1 | TRP | A | 634 | 1217 | 708 | 954 | 12 | −117 | 75 | | N |
| ATOM | 2069 | CE2 | TRP | A | 634 | −14.704 | −19.846 | 31.284 | 1.00 | 11.40 | | | C |
| ANISOU | 2069 | CE2 | TRP | A | 634 | 1712 | 1201 | 1419 | 12 | −105 | 77 | | C |
| ATOM | 2070 | CD2 | TRP | A | 634 | −14.314 | −20.055 | 32.630 | 1.00 | 8.26 | | | C |
| ANISOU | 2070 | CD2 | TRP | A | 634 | 1320 | 840 | 979 | 27 | −114 | 89 | | C |
| ATOM | 2071 | CE3 | TRP | A | 634 | −15.258 | −19.863 | 33.652 | 1.00 | 10.03 | | | C |
| ANISOU | 2071 | CE3 | TRP | A | 634 | 1553 | 1094 | 1166 | 28 | −96 | 89 | | C |
| ATOM | 2072 | CZ3 | TRP | A | 634 | −16.534 | −19.462 | 33.306 | 1.00 | 8.49 | | | C |
| ANISOU | 2072 | CZ3 | TRP | A | 634 | 1355 | 880 | 990 | 17 | −70 | 75 | | C |
| ATOM | 2073 | CH2 | TRP | A | 634 | −16.894 | −19.268 | 31.950 | 1.00 | 5.81 | | | C |
| ANISOU | 2073 | CH2 | TRP | A | 634 | 1007 | 501 | 701 | 5 | −70 | 69 | | C |
| ATOM | 2074 | CZ2 | TRP | A | 634 | −15.989 | −19.450 | 30.934 | 1.00 | 12.73 | | | C |
| ANISOU | 2074 | CZ2 | TRP | A | 634 | 1879 | 1358 | 1599 | 1 | −87 | 71 | | C |
| ATOM | 2075 | C | TRP | A | 634 | −10.222 | −22.132 | 34.812 | 1.00 | 9.00 | | | C |
| ANISOU | 2075 | C | TRP | A | 634 | 1365 | 1039 | 1015 | 116 | −218 | 195 | | C |
| ATOM | 2076 | O | TRP | A | 634 | −9.910 | −21.184 | 35.540 | 1.00 | 8.77 | | | O |
| ANISOU | 2076 | O | TRP | A | 634 | 1327 | 1073 | 933 | 106 | −244 | 157 | | O |
| ATOM | 2077 | N | HIS | A | 635 | −9.850 | −23.383 | 35.078 | 1.00 | 8.03 | | | N |
| ANISOU | 2077 | N | HIS | A | 635 | 1237 | 901 | 912 | 150 | −224 | 266 | | N |
| ATOM | 2078 | CA | HIS | A | 635 | −8.962 | −23.656 | 36.202 | 1.00 | 14.42 | | | C |
| ANISOU | 2078 | CA | HIS | A | 635 | 2025 | 1781 | 1673 | 183 | −270 | 315 | | C |
| ATOM | 2079 | CB | HIS | A | 635 | −8.555 | −25.133 | 36.237 | 1.00 | 17.21 | | | C |
| ANISOU | 2079 | CB | HIS | A | 635 | 2367 | 2087 | 2083 | 229 | −272 | 406 | | C |
| ATOM | 2080 | CG | HIS | A | 635 | −9.577 | −26.026 | 36.860 | 1.00 | 28.23 | | | C |
| ANISOU | 2080 | CG | HIS | A | 635 | 3796 | 3462 | 3469 | 233 | −242 | 492 | | C |
| ATOM | 2081 | ND1 | HIS | A | 635 | −10.480 | −26.758 | 36.114 | 1.00 | 29.37 | | | N |
| ANISOU | 2081 | ND1 | HIS | A | 635 | 3961 | 3507 | 3689 | 218 | −191 | 500 | | N |
| ATOM | 2082 | CE1 | HIS | A | 635 | −11.264 | −27.444 | 36.929 | 1.00 | 28.32 | | | C |
| ANISOU | 2082 | CE1 | HIS | A | 635 | 3850 | 3374 | 3538 | 219 | −169 | 587 | | C |
| ATOM | 2083 | NE2 | HIS | A | 635 | −10.902 | −27.182 | 38.173 | 1.00 | 34.73 | | | N |
| ANISOU | 2083 | NE2 | HIS | A | 635 | 4657 | 4291 | 4249 | 237 | −204 | 641 | | N |
| ATOM | 2084 | CD2 | HIS | A | 635 | −9.850 | −26.297 | 38.158 | 1.00 | 23.63 | | | C |
| ANISOU | 2084 | CD2 | HIS | A | 635 | 3224 | 2950 | 2805 | 246 | −254 | 575 | | C |
| ATOM | 2085 | C | HIS | A | 635 | −7.719 | −22.791 | 36.094 | 1.00 | 18.85 | | | C |
| ANISOU | 2085 | C | HIS | A | 635 | 2543 | 2388 | 2230 | 179 | −312 | 255 | | C |
| ATOM | 2086 | O | HIS | A | 635 | −7.138 | −22.655 | 35.014 | 1.00 | 11.46 | | | O |
| ANISOU | 2086 | O | HIS | A | 635 | 1585 | 1407 | 1361 | 173 | −304 | 216 | | O |
| ATOM | 2087 | N | GLU | A | 636 | −7.300 | −22.202 | 37.216 | 1.00 | 14.82 | | | N |
| ANISOU | 2087 | N | GLU | A | 636 | 2018 | 1976 | 1639 | 179 | −357 | 243 | | N |
| ATOM | 2088 | CA | GLU | A | 636 | −6.079 | −21.403 | 37.170 | 1.00 | 14.60 | | | C |
| ANISOU | 2088 | CA | GLU | A | 636 | 1939 | 1992 | 1615 | 169 | −401 | 182 | | C |
| ATOM | 2089 | CB | GLU | A | 636 | −5.802 | −20.766 | 38.534 | 1.00 | 14.28 | | | C |
| ANISOU | 2089 | CB | GLU | A | 636 | 1883 | 2073 | 1471 | 161 | −451 | 154 | | C |
| ATOM | 2090 | CG | GLU | A | 636 | −4.534 | −19.911 | 38.567 | 1.00 | 14.32 | | | C |
| ANISOU | 2090 | CG | GLU | A | 636 | 1826 | 2127 | 1486 | 141 | −502 | 80 | | C |
| ATOM | 2091 | CD | GLU | A | 636 | −4.402 | −19.109 | 39.865 | 1.00 | 31.85 | | | C |
| ANISOU | 2091 | CD | GLU | A | 636 | 4033 | 4471 | 3600 | 119 | −550 | 18 | | C |
| ATOM | 2092 | OE1 | GLU | A | 636 | −5.121 | −18.095 | 40.029 | 1.00 | 34.72 | | | O |
| ANISOU | 2092 | OE1 | GLU | A | 636 | 4422 | 4829 | 3942 | 78 | −524 | −68 | | O |
| ATOM | 2093 | OE2 | GLU | A | 636 | −3.589 | −19.501 | 40.731 | 1.00 | 33.59 | | | O |
| ANISOU | 2093 | OE2 | GLU | A | 636 | 4211 | 4796 | 3757 | 146 | −614 | 53 | | O |
| ATOM | 2094 | C | GLU | A | 636 | −4.893 | −22.250 | 36.715 | 1.00 | 15.59 | | | C |
| ANISOU | 2094 | C | GLU | A | 636 | 2016 | 2098 | 1808 | 209 | −423 | 224 | | C |
| ATOM | 2095 | O | GLU | A | 636 | −4.064 | −21.798 | 35.919 | 1.00 | 15.31 | | | O |
| ANISOU | 2095 | O | GLU | A | 636 | 1942 | 2046 | 1831 | 195 | −425 | 172 | | O |
| ATOM | 2096 | N | LYS | A | 637 | −4.807 | −23.486 | 37.190 | 1.00 | 14.32 | | | N |
| ANISOU | 2096 | N | LYS | A | 637 | 1854 | 1936 | 1651 | 261 | −435 | 322 | | N |
| ATOM | 2097 | CA | LYS | A | 637 | −3.757 | −24.401 | 36.747 | 1.00 | 16.78 | | | C |
| ANISOU | 2097 | CA | LYS | A | 637 | 2115 | 2214 | 2046 | 311 | −450 | 364 | | C |
| ATOM | 2098 | CB | LYS | A | 637 | −3.505 | −25.458 | 37.813 | 1.00 | 21.63 | | | C |
| ANISOU | 2098 | CB | LYS | A | 637 | 2717 | 2867 | 2636 | 371 | −491 | 484 | | C |
| ATOM | 2099 | CG | LYS | A | 637 | −2.994 | −24.867 | 39.113 | 1.00 | 27.69 | | | C |
| ANISOU | 2099 | CG | LYS | A | 637 | 3454 | 3780 | 3284 | 371 | −564 | 493 | | C |
| ATOM | 2100 | CD | LYS | A | 637 | −2.699 | −25.943 | 40.143 | 1.00 | 33.60 | | | C |
| ANISOU | 2100 | CD | LYS | A | 637 | 4191 | 4572 | 4005 | 431 | −605 | 623 | | C |
| ATOM | 2101 | CE | LYS | A | 637 | −2.263 | −25.334 | 41.478 | 1.00 | 36.60 | | | C |
| ANISOU | 2101 | CE | LYS | A | 637 | 4552 | 5099 | 4254 | 414 | −668 | 605 | | C |
| ATOM | 2102 | NZ | LYS | A | 637 | −1.998 | −26.400 | 42.488 | 1.00 | 40.30 | | | N |
| ANISOU | 2102 | NZ | LYS | A | 637 | 5025 | 5586 | 4702 | 455 | −690 | 710 | | N |
| ATOM | 2103 | C | LYS | A | 637 | −4.146 | −25.043 | 35.416 | 1.00 | 13.38 | | | C |
| ANISOU | 2103 | C | LYS | A | 637 | 1703 | 1661 | 1718 | 312 | −386 | 351 | | C |
| ATOM | 2104 | O | LYS | A | 637 | −5.146 | −25.763 | 35.337 | 1.00 | 13.36 | | | O |
| ANISOU | 2104 | O | LYS | A | 637 | 1748 | 1596 | 1733 | 315 | −348 | 393 | | O |
| ATOM | 2105 | N | ALA | A | 638 | −3.348 | −24.785 | 34.373 | 1.00 | 13.44 | | | N |
| ANISOU | 2105 | N | ALA | A | 638 | 1671 | 1646 | 1791 | 306 | −373 | 288 | | N |
| ATOM | 2106 | CA | ALA | A | 638 | −3.696 | −25.248 | 33.031 | 1.00 | 13.03 | | | C |
| ANISOU | 2106 | CA | ALA | A | 638 | 1634 | 1504 | 1815 | 298 | −311 | 253 | | C |
| ATOM | 2107 | CB | ALA | A | 638 | −2.644 | −24.794 | 32.024 | 1.00 | 9.59 | | | C |
| ANISOU | 2107 | CB | ALA | A | 638 | 1142 | 1077 | 1425 | 287 | −299 | 185 | | C |
| ATOM | 2108 | C | ALA | A | 638 | −3.853 | −26.761 | 32.978 | 1.00 | 12.43 | | | C |
| ANISOU | 2108 | C | ALA | A | 638 | 1561 | 1346 | 1815 | 351 | −291 | 314 | | C |
| ATOM | 2109 | O | ALA | A | 638 | −4.722 | −27.269 | 32.265 | 1.00 | 12.46 | | | O |
| ANISOU | 2109 | O | ALA | A | 638 | 1603 | 1274 | 1859 | 335 | −241 | 297 | | O |
| ATOM | 2110 | N | ASP | A | 639 | −3.024 | −27.500 | 33.732 | 1.00 | 13.83 | | | N |
| ANISOU | 2110 | N | ASP | A | 639 | 1695 | 1535 | 2022 | 413 | −332 | 387 | | N |
| ATOM | 2111 | CA | ASP | A | 639 | −3.047 | −28.961 | 33.681 | 1.00 | 20.33 | | | C |
| ANISOU | 2111 | CA | ASP | A | 639 | 2515 | 2263 | 2948 | 470 | −312 | 453 | | C |
| ATOM | 2112 | CB | ASP | A | 639 | −1.770 | −29.556 | 34.298 | 1.00 | 31.27 | | | C |
| ANISOU | 2112 | CB | ASP | A | 639 | 3828 | 3672 | 4383 | 547 | −365 | 523 | | C |
| ATOM | 2113 | CG | ASP | A | 639 | −1.600 | −29.223 | 35.784 | 1.00 | 49.85 | | | C |
| ANISOU | 2113 | CG | ASP | A | 639 | 6179 | 6139 | 6624 | 557 | −435 | 607 | | C |
| ATOM | 2114 | OD1 | ASP | A | 639 | −2.583 | −29.285 | 36.554 | 1.00 | 54.27 | | | O |
| ANISOU | 2114 | OD1 | ASP | A | 639 | 6801 | 6715 | 7104 | 532 | −429 | 655 | | O |
| ATOM | 2115 | OD2 | ASP | A | 639 | −0.456 | −28.912 | 36.185 | 1.00 | 60.46 | | | O |
| ANISOU | 2115 | OD2 | ASP | A | 639 | 7455 | 7567 | 7951 | 586 | −493 | 609 | | O |
| ATOM | 2116 | C | ASP | A | 639 | −4.280 | −29.565 | 34.348 | 1.00 | 20.42 | | | C |
| ANISOU | 2116 | C | ASP | A | 639 | 2588 | 2230 | 2940 | 463 | −297 | 535 | | C |
| ATOM | 2117 | O | ASP | A | 639 | −4.543 | −30.756 | 34.157 | 1.00 | 20.36 | | | O |
| ANISOU | 2117 | O | ASP | A | 639 | 2595 | 2133 | 3010 | 481 | −258 | 555 | | O |
| ATOM | 2118 | N | AGLU | A | 640 | −5.036 | −28.787 | 35.111 | 0.34 | 15.09 | | | N |
| ANISOU | 2118 | N | AGLU | A | 640 | 1955 | 1631 | 2149 | 421 | −312 | 550 | | N |
| ATOM | 2119 | CA | AGLU | A | 640 | −6.292 | −29.289 | 35.638 | 0.34 | 15.36 | | | C |
| ANISOU | 2119 | CA | AGLU | A | 640 | 2044 | 1632 | 2161 | 402 | −282 | 613 | | C |
| ATOM | 2120 | CB | AGLU | A | 640 | −6.625 | −28.591 | 36.961 | 0.34 | 18.45 | | | C |
| ANISOU | 2120 | CB | AGLU | A | 640 | 2454 | 2143 | 2412 | 387 | −318 | 662 | | C |
| ATOM | 2121 | CG | AGLU | A | 640 | −5.528 | −28.736 | 38.014 | 0.34 | 23.81 | | | C |
| ANISOU | 2121 | CG | AGLU | A | 640 | 3094 | 2912 | 3039 | 430 | −381 | 715 | | C |
| ATOM | 2122 | CD | AGLU | A | 640 | −5.309 | −30.175 | 38.450 | 0.34 | 27.01 | | | C |
| ANISOU | 2122 | CD | AGLU | A | 640 | 3500 | 3257 | 3507 | 470 | −372 | 795 | | C |
| ATOM | 2123 | OE1 | AGLU | A | 640 | −6.290 | −30.947 | 38.483 | 0.34 | 27.65 | | | O |
| ANISOU | 2123 | OE1 | AGLU | A | 640 | 3622 | 3265 | 3619 | 451 | −322 | 829 | | O |
| ATOM | 2124 | OE2 | AGLU | A | 640 | −4.153 | −30.538 | 38.757 | 0.34 | 32.30 | | | O |
| ANISOU | 2124 | OE2 | AGLU | A | 640 | 4121 | 3951 | 4198 | 520 | −417 | 825 | | O |
| ATOM | 2125 | C | AGLU | A | 640 | −7.442 | −29.133 | 34.654 | 0.34 | 14.51 | | | C |
| ANISOU | 2125 | C | AGLU | A | 640 | 1976 | 1456 | 2079 | 347 | −223 | 539 | | C |
| ATOM | 2126 | O | AGLU | A | 640 | −8.495 | −29.742 | 34.860 | 0.34 | 12.58 | | | O |
| ANISOU | 2126 | O | AGLU | A | 640 | 1769 | 1166 | 1845 | 327 | −186 | 573 | | O |
| ATOM | 2127 | N | BGLU | A | 640 | −5.047 | −28.783 | 35.105 | 0.66 | 14.31 | | | N |
| ANISOU | 2127 | N | BGLU | A | 640 | 1856 | 1532 | 2050 | 420 | −311 | 549 | | N |
| ATOM | 2128 | CA | BGLU | A | 640 | −6.309 | −29.264 | 35.652 | 0.66 | 14.77 | | | C |
| ANISOU | 2128 | CA | BGLU | A | 640 | 1970 | 1559 | 2084 | 401 | −282 | 613 | | C |
| ATOM | 2129 | CB | BGLU | A | 640 | −6.570 | −28.645 | 37.029 | 0.66 | 18.14 | | | C |
| ANISOU | 2129 | CB | BGLU | A | 640 | 2413 | 2107 | 2372 | 390 | −321 | 667 | | C |
| ATOM | 2130 | CG | BGLU | A | 640 | −5.565 | −29.069 | 38.095 | 0.66 | 24.73 | | | C |
| ANISOU | 2130 | CG | BGLU | A | 640 | 3215 | 3012 | 3170 | 437 | −375 | 732 | | C |
| ATOM | 2131 | CD | BGLU | A | 640 | −5.800 | −28.377 | 39.427 | 0.66 | 26.40 | | | C |
| ANISOU | 2131 | CD | BGLU | A | 640 | 3440 | 3359 | 3230 | 417 | −413 | 760 | | C |
| ATOM | 2132 | OE1 | BGLU | A | 640 | −6.644 | −27.454 | 39.483 | 0.66 | 24.37 | | | O |
| ANISOU | 2132 | OE1 | BGLU | A | 640 | 3214 | 3149 | 2899 | 372 | −397 | 718 | | O |
| ATOM | 2133 | OE2 | BGLU | A | 640 | −5.131 | −28.750 | 40.413 | 0.66 | 31.09 | | | O |
| ANISOU | 2133 | OE2 | BGLU | A | 640 | 4014 | 4019 | 3781 | 447 | −458 | 818 | | O |
| ATOM | 2134 | C | BGLU | A | 640 | −7.490 | −28.986 | 34.734 | 0.66 | 15.29 | | | C |
| ANISOU | 2134 | C | BGLU | A | 640 | 2078 | 1568 | 2163 | 343 | −225 | 538 | | C |
| ATOM | 2135 | O | BGLU | A | 640 | −8.619 | −29.332 | 35.084 | 0.66 | 12.42 | | | O |
| ANISOU | 2135 | O | BGLU | A | 640 | 1753 | 1178 | 1786 | 319 | −196 | 578 | | O |
| ATOM | 2136 | N | ARG | A | 641 | −7.265 | −28.363 | 33.580 | 1.00 | 12.65 | | | N |
| ANISOU | 2136 | N | ARG | A | 641 | 1732 | 1226 | 1846 | 316 | −209 | 429 | | N |
| ATOM | 2137 | CA | ARG | A | 641 | −8.358 | −28.184 | 32.635 | 1.00 | 14.55 | | | C |
| ANISOU | 2137 | CA | ARG | A | 641 | 2006 | 1423 | 2098 | 265 | −162 | 362 | | C |
| ATOM | 2138 | CB | ARG | A | 641 | −8.098 | −26.991 | 31.714 | 1.00 | 11.17 | | | C |
| ANISOU | 2138 | CB | ARG | A | 641 | 1569 | 1041 | 1634 | 229 | −161 | 267 | | C |
| ATOM | 2139 | CG | ARG | A | 641 | −7.889 | −25.677 | 32.450 | 1.00 | 11.52 | | | C |
| ANISOU | 2139 | CG | ARG | A | 641 | 1613 | 1178 | 1587 | 212 | −197 | 260 | | C |
| ATOM | 2140 | CD | ARG | A | 641 | −7.473 | −24.497 | 31.527 | 1.00 | 13.45 | | | C |
| ANISOU | 2140 | CD | ARG | A | 641 | 1841 | 1452 | 1819 | 177 | −195 | 181 | | C |
| ATOM | 2141 | NE | ARG | A | 641 | −6.778 | −23.492 | 32.326 | 1.00 | 12.13 | | | N |
| ANISOU | 2141 | NE | ARG | A | 641 | 1652 | 1358 | 1599 | 172 | −237 | 173 | | N |
| ATOM | 2142 | CZ | ARG | A | 641 | −5.836 | −22.666 | 31.866 | 1.00 | 10.54 | | | C |
| ANISOU | 2142 | CZ | ARG | A | 641 | 1413 | 1187 | 1407 | 156 | −248 | 127 | | C |
| ATOM | 2143 | NH1 | ARG | A | 641 | −5.460 | −22.680 | 30.585 | 1.00 | 8.08 | | | N |
| ANISOU | 2143 | NH1 | ARG | A | 641 | 1081 | 848 | 1141 | 144 | −216 | 90 | | N |
| ATOM | 2144 | NH2 | ARG | A | 641 | −5.241 | −21.843 | 32.708 | 1.00 | 8.07 | | | N |
| ANISOU | 2144 | NH2 | ARG | A | 641 | 1077 | 937 | 1054 | 147 | −290 | 113 | | N |
| ATOM | 2145 | C | ARG | A | 641 | −8.529 | −29.459 | 31.810 | 1.00 | 14.26 | | | C |
| ANISOU | 2145 | C | ARG | A | 641 | 1966 | 1274 | 2179 | 277 | −121 | 349 | | C |
| ATOM | 2146 | O | ARG | A | 641 | −7.563 | −30.184 | 31.564 | 1.00 | 10.24 | | | O |
| ANISOU | 2146 | O | ARG | A | 641 | 1419 | 720 | 1751 | 324 | −123 | 352 | | O |
| ATOM | 2147 | N | PRO | A | 642 | −9.750 | −29.765 | 31.384 | 1.00 | 10.70 | | | N |
| ANISOU | 2147 | N | PRO | A | 642 | 1545 | 772 | 1748 | 237 | −83 | 327 | | N |
| ATOM | 2148 | CA | PRO | A | 642 | −9.980 | −31.001 | 30.624 | 1.00 | 11.35 | | | C |
| ANISOU | 2148 | CA | PRO | A | 642 | 1622 | 758 | 1932 | 235 | −43 | 289 | | C |
| ATOM | 2149 | CB | PRO | A | 642 | −11.510 | −31.093 | 30.580 | 1.00 | 9.72 | | | C |
| ANISOU | 2149 | CB | PRO | A | 642 | 1450 | 538 | 1706 | 178 | −16 | 281 | | C |
| ATOM | 2150 | CG | PRO | A | 642 | −11.931 | −29.652 | 30.540 | 1.00 | 20.44 | | | C |
| ANISOU | 2150 | CG | PRO | A | 642 | 2821 | 1974 | 2973 | 148 | −32 | 261 | | C |
| ATOM | 2151 | CD | PRO | A | 642 | −10.967 | −28.950 | 31.492 | 1.00 | 11.96 | | | C |
| ANISOU | 2151 | CD | PRO | A | 642 | 1739 | 976 | 1830 | 184 | −74 | 313 | | C |
| ATOM | 2152 | C | PRO | A | 642 | −9.389 | −30.904 | 29.224 | 1.00 | 14.78 | | | C |
| ANISOU | 2152 | C | PRO | A | 642 | 2029 | 1173 | 2414 | 235 | −25 | 180 | | C |
| ATOM | 2153 | O | PRO | A | 642 | −8.906 | −29.858 | 28.787 | 1.00 | 12.78 | | | O |
| ANISOU | 2153 | O | PRO | A | 642 | 1765 | 1002 | 2089 | 223 | −39 | 133 | | O |
| ATOM | 2154 | N | THR | A | 643 | −9.421 | −32.034 | 28.522 | 1.00 | 11.84 | | | N |
| ANISOU | 2154 | N | THR | A | 643 | 1644 | 734 | 2119 | 235 | 9 | 124 | | N |
| ATOM | 2155 | CA | THR | A | 643 | −9.155 | −32.103 | 27.091 | 1.00 | 13.59 | | | C |
| ANISOU | 2155 | CA | THR | A | 643 | 1844 | 946 | 2375 | 221 | 39 | 1 | | C |
| ATOM | 2156 | CB | THR | A | 643 | −8.559 | −33.457 | 26.725 | 1.00 | 11.54 | | | C |
| ANISOU | 2156 | CB | THR | A | 643 | 1553 | 620 | 2212 | 253 | 66 | −36 | | C |
| ATOM | 2157 | OG1 | THR | A | 643 | −9.526 | −34.464 | 27.049 | 1.00 | 12.12 | | | O |
| ANISOU | 2157 | OG1 | THR | A | 643 | 1647 | 632 | 2324 | 227 | 79 | −9 | | O |
| ATOM | 2158 | CG2 | THR | A | 643 | −7.276 | −33.730 | 27.518 | 1.00 | 12.11 | | | C |
| ANISOU | 2158 | CG2 | THR | A | 643 | 1592 | 686 | 2322 | 323 | 43 | 37 | | C |
| ATOM | 2159 | C | THR | A | 643 | −10.460 | −31.908 | 26.326 | 1.00 | 14.31 | | | C |
| ANISOU | 2159 | C | THR | A | 643 | 1960 | 1045 | 2433 | 154 | 58 | −65 | | C |
| ATOM | 2160 | O | THR | A | 643 | −11.551 | −31.944 | 26.907 | 1.00 | 11.26 | | | O |
| ANISOU | 2160 | O | THR | A | 643 | 1601 | 653 | 2023 | 123 | 53 | −15 | | O |
| ATOM | 2161 | N | PHE | A | 644 | −10.353 | −31.739 | 24.996 | 1.00 | 9.66 | | | N |
| ANISOU | 2161 | N | PHE | A | 644 | 1660 | 711 | 1301 | 23 | −108 | 106 | | N |
| ATOM | 2162 | CA | PHE | A | 644 | −11.582 | −31.609 | 24.207 | 1.00 | 10.00 | | | C |
| ANISOU | 2162 | CA | PHE | A | 644 | 1806 | 705 | 1288 | 65 | −51 | −45 | | C |
| ATOM | 2163 | CB | PHE | A | 644 | −11.276 | −31.074 | 22.803 | 1.00 | 9.23 | | | C |
| ANISOU | 2163 | CB | PHE | A | 644 | 1730 | 617 | 1161 | 59 | 2 | −118 | | C |
| ATOM | 2164 | CG | PHE | A | 644 | −11.013 | −29.590 | 22.786 | 1.00 | 10.29 | | | C |
| ANISOU | 2164 | CG | PHE | A | 644 | 1916 | 794 | 1198 | −5 | −59 | −102 | | C |
| ATOM | 2165 | CD1 | PHE | A | 644 | −12.054 | −28.689 | 22.992 | 1.00 | 9.95 | | | C |
| ANISOU | 2165 | CD1 | PHE | A | 644 | 1948 | 708 | 1125 | −17 | −89 | −127 | | C |
| ATOM | 2166 | CE1 | PHE | A | 644 | −11.813 | −27.318 | 23.000 | 1.00 | 11.56 | | | C |
| ANISOU | 2166 | CE1 | PHE | A | 644 | 2200 | 890 | 1303 | −73 | −116 | −116 | | C |
| ATOM | 2167 | CZ | PHE | A | 644 | −10.522 | −26.842 | 22.809 | 1.00 | 10.03 | | | C |
| ANISOU | 2167 | CZ | PHE | A | 644 | 1998 | 763 | 1051 | −140 | −141 | −77 | | C |
| ATOM | 2168 | CE2 | PHE | A | 644 | −9.484 | −27.733 | 22.613 | 1.00 | 10.13 | | | C |
| ANISOU | 2168 | CE2 | PHE | A | 644 | 1918 | 856 | 1076 | −131 | −122 | −40 | | C |
| ATOM | 2169 | CD2 | PHE | A | 644 | −9.729 | −29.096 | 22.610 | 1.00 | 10.61 | | | C |
| ANISOU | 2169 | CD2 | PHE | A | 644 | 1912 | 894 | 1225 | −51 | −67 | −54 | | C |
| ATOM | 2170 | C | PHE | A | 644 | −12.342 | −32.926 | 24.150 | 1.00 | 12.40 | | | C |
| ANISOU | 2170 | C | PHE | A | 644 | 2098 | 939 | 1675 | 118 | −7 | −70 | | C |
| ATOM | 2171 | O | PHE | A | 644 | −13.577 | −32.926 | 24.072 | 1.00 | 12.90 | | | O |
| ANISOU | 2171 | O | PHE | A | 644 | 2200 | 1026 | 1677 | 108 | −8 | −128 | | O |
| ATOM | 2172 | N | LYS | A | 645 | −11.631 | −34.051 | 24.228 | 1.00 | 10.79 | | | N |
| ANISOU | 2172 | N | LYS | A | 645 | 1785 | 664 | 1651 | 167 | 36 | 0 | | N |
| ATOM | 2173 | CA | LYS | A | 645 | −12.293 | −35.348 | 24.336 | 1.00 | 15.63 | | | C |
| ANISOU | 2173 | CA | LYS | A | 645 | 2392 | 1175 | 2372 | 203 | 79 | −8 | | C |
| ATOM | 2174 | CB | LYS | A | 645 | −11.247 | −36.463 | 24.292 | 1.00 | 17.10 | | | C |
| ANISOU | 2174 | CB | LYS | A | 645 | 2410 | 1260 | 2827 | 268 | 150 | 80 | | C |
| ATOM | 2175 | CG | LYS | A | 645 | −11.804 | −37.870 | 24.161 | 1.00 | 25.72 | | | C |
| ANISOU | 2175 | CG | LYS | A | 645 | 3492 | 2285 | 3996 | 280 | 209 | 37 | | C |
| ATOM | 2176 | CD | LYS | A | 645 | −10.703 | −38.822 | 23.698 | 1.00 | 31.33 | | | C |
| ANISOU | 2176 | CD | LYS | A | 645 | 4032 | 2880 | 4993 | 339 | 366 | 54 | | C |
| ATOM | 2177 | CE | LYS | A | 645 | −11.109 | −40.277 | 23.836 | 1.00 | 37.31 | | | C |
| ANISOU | 2177 | CE | LYS | A | 645 | 4756 | 3525 | 5896 | 348 | 422 | 62 | | C |
| ATOM | 2178 | NZ | LYS | A | 645 | −12.339 | −40.585 | 23.050 | 1.00 | 40.46 | | | N |
| ANISOU | 2178 | NZ | LYS | A | 645 | 5338 | 3948 | 6088 | 273 | 477 | −136 | | N |
| ATOM | 2179 | C | LYS | A | 645 | −13.130 | −35.437 | 25.610 | 1.00 | 14.49 | | | C |
| ANISOU | 2179 | C | LYS | A | 645 | 2281 | 1089 | 2135 | 143 | −44 | 76 | | C |
| ATOM | 2180 | O | LYS | A | 645 | −14.269 | −35.913 | 25.580 | 1.00 | 12.43 | | | O |
| ANISOU | 2180 | O | LYS | A | 645 | 2081 | 815 | 1826 | 134 | −28 | 8 | | O |
| ATOM | 2181 | N | ILE | A | 646 | −12.583 | −34.996 | 26.744 | 1.00 | 10.36 | | | N |
| ANISOU | 2181 | N | ILE | A | 646 | 1715 | 661 | 1559 | 52 | −159 | 223 | | N |
| ATOM | 2182 | CA | ILE | A | 646 | −13.350 | −35.052 | 27.985 | 1.00 | 11.12 | | | C |
| ANISOU | 2182 | CA | ILE | A | 646 | 1865 | 845 | 1516 | −84 | −242 | 273 | | C |
| ATOM | 2183 | CB | ILE | A | 646 | −12.444 | −34.769 | 29.190 | 1.00 | 11.88 | | | C |
| ANISOU | 2183 | CB | ILE | A | 646 | 1904 | 1073 | 1536 | −276 | −381 | 480 | | C |
| ATOM | 2184 | CG1 | ILE | A | 646 | −11.463 | −35.933 | 29.405 | 1.00 | 18.70 | | | C |
| ANISOU | 2184 | CG1 | ILE | A | 646 | 2563 | 1874 | 2668 | −249 | −486 | 774 | | C |
| ATOM | 2185 | CD1 | ILE | A | 646 | −10.303 | −35.620 | 30.313 | 1.00 | 25.18 | | | C |
| ANISOU | 2185 | CD1 | ILE | A | 646 | 3253 | 2859 | 3453 | −438 | −645 | 1024 | | C |
| ATOM | 2186 | CG2 | ILE | A | 646 | −13.311 | −34.544 | 30.413 | 1.00 | 12.32 | | | C |
| ANISOU | 2186 | CG2 | ILE | A | 646 | 2071 | 1258 | 1352 | −502 | −413 | 449 | | C |
| ATOM | 2187 | C | ILE | A | 646 | −14.535 | −34.091 | 27.928 | 1.00 | 10.34 | | | C |
| ANISOU | 2187 | C | ILE | A | 646 | 1898 | 785 | 1244 | −108 | −170 | 75 | | C |
| ATOM | 2188 | O | ILE | A | 646 | −15.655 | −34.443 | 28.320 | 1.00 | 11.02 | | | O |
| ANISOU | 2188 | O | ILE | A | 646 | 2025 | 882 | 1282 | −140 | −149 | 23 | | O |
| ATOM | 2189 | N | LEU | A | 647 | −14.322 | −32.875 | 27.421 | 1.00 | 9.33 | | | N |
| ANISOU | 2189 | N | LEU | A | 647 | 1814 | 662 | 1070 | −89 | −125 | −17 | | N |
| ATOM | 2190 | CA | LEU | A | 647 | −15.437 | −31.935 | 27.261 | 1.00 | 10.24 | | | C |
| ANISOU | 2190 | CA | LEU | A | 647 | 1997 | 750 | 1144 | −75 | −42 | −161 | | C |
| ATOM | 2191 | CB | LEU | A | 647 | −14.933 | −30.621 | 26.659 | 1.00 | 9.76 | | | C |
| ANISOU | 2191 | CB | LEU | A | 647 | 1933 | 683 | 1092 | −55 | −18 | −193 | | C |
| ATOM | 2192 | CG | LEU | A | 647 | −14.078 | −29.705 | 27.543 | 1.00 | 12.26 | | | C |
| ANISOU | 2192 | CG | LEU | A | 647 | 2336 | 1025 | 1298 | −209 | −15 | −208 | | C |
| ATOM | 2193 | CD1 | LEU | A | 647 | −13.450 | −28.604 | 26.688 | 1.00 | 9.72 | | | C |
| ANISOU | 2193 | CD1 | LEU | A | 647 | 2005 | 679 | 1011 | −169 | −13 | −210 | | C |
| ATOM | 2194 | CD2 | LEU | A | 647 | −14.907 | −29.089 | 28.665 | 1.00 | 18.67 | | | C |
| ANISOU | 2194 | CD2 | LEU | A | 647 | 3174 | 1865 | 2057 | −325 | 104 | −311 | | C |
| ATOM | 2195 | C | LEU | A | 647 | −16.542 | −32.515 | 26.379 | 1.00 | 14.20 | | | C |
| ANISOU | 2195 | C | LEU | A | 647 | 2425 | 1247 | 1721 | 23 | −18 | −180 | | C |
| ATOM | 2196 | O | LEU | A | 647 | −17.740 | −32.355 | 26.669 | 1.00 | 10.12 | | | O |
| ANISOU | 2196 | O | LEU | A | 647 | 1891 | 735 | 1218 | 18 | 25 | −224 | | O |
| ATOM | 2197 | N | LEU | A | 648 | −16.161 | −33.151 | 25.273 | 1.00 | 9.22 | | | N |
| ANISOU | 2197 | N | LEU | A | 648 | 1748 | 615 | 1141 | 73 | −28 | −155 | | N |
| ATOM | 2198 | CA | LEU | A | 648 | −17.166 | −33.734 | 24.385 | 1.00 | 11.31 | | | C |
| ANISOU | 2198 | CA | LEU | A | 648 | 1991 | 889 | 1416 | 77 | −16 | −173 | | C |
| ATOM | 2199 | CB | LEU | A | 648 | −16.488 | −34.389 | 23.182 | 1.00 | 8.13 | | | C |
| ANISOU | 2199 | CB | LEU | A | 648 | 1591 | 467 | 1030 | 64 | 21 | −201 | | C |
| ATOM | 2200 | CG | LEU | A | 648 | −17.421 | −34.982 | 22.121 | 1.00 | 13.01 | | | C |
| ANISOU | 2200 | CG | LEU | A | 648 | 2272 | 1083 | 1586 | −13 | 37 | −240 | | C |
| ATOM | 2201 | CD1 | LEU | A | 648 | −18.391 | −33.907 | 21.626 | 1.00 | 11.90 | | | C |
| ANISOU | 2201 | CD1 | LEU | A | 648 | 2200 | 941 | 1380 | −64 | −47 | −198 | | C |
| ATOM | 2202 | CD2 | LEU | A | 648 | −16.615 | −35.547 | 20.958 | 1.00 | 15.57 | | | C |
| ANISOU | 2202 | CD2 | LEU | A | 648 | 2624 | 1395 | 1898 | −83 | 132 | −321 | | C |
| ATOM | 2203 | C | LEU | A | 648 | −18.027 | −34.743 | 25.128 | 1.00 | 10.28 | | | C |
| ANISOU | 2203 | C | LEU | A | 648 | 1882 | 742 | 1282 | 60 | −20 | −169 | | C |
| ATOM | 2204 | O | LEU | A | 648 | −19.264 | −34.730 | 25.015 | 1.00 | 12.96 | | | O |
| ANISOU | 2204 | O | LEU | A | 648 | 2235 | 1084 | 1606 | 46 | −22 | −182 | | O |
| ATOM | 2205 | N | ASER | A | 649 | −17.395 | −35.638 | 25.886 | 0.34 | 12.17 | | | N |
| ANISOU | 2205 | N | ASER | A | 649 | 2143 | 927 | 1554 | 51 | −38 | −126 | | N |
| ATOM | 2206 | CA | ASER | A | 649 | −18.142 | −36.603 | 26.683 | 0.34 | 12.22 | | | C |
| ANISOU | 2206 | CA | ASER | A | 649 | 2199 | 897 | 1549 | 1 | −68 | −94 | | C |
| ATOM | 2207 | CB | ASER | A | 649 | −17.172 | −37.532 | 27.412 | 0.34 | 12.39 | | | C |
| ANISOU | 2207 | CB | ASER | A | 649 | 2173 | 863 | 1672 | −22 | −128 | 49 | | C |
| ATOM | 2208 | OG | ASER | A | 649 | −17.846 | −38.329 | 28.363 | 0.34 | 12.93 | | | O |
| ANISOU | 2208 | OG | ASER | A | 649 | 2244 | 964 | 1705 | −121 | −189 | 129 | | O |
| ATOM | 2209 | C | ASER | A | 649 | −19.069 | −35.903 | 27.669 | 0.34 | 12.17 | | | C |
| ANISOU | 2209 | C | ASER | A | 649 | 2206 | 981 | 1437 | −74 | −58 | −130 | | C |
| ATOM | 2210 | O | ASER | A | 649 | −20.221 | −36.315 | 27.844 | 0.34 | 13.58 | | | O |
| ANISOU | 2210 | O | ASER | A | 649 | 2382 | 1189 | 1589 | −108 | −42 | −151 | | O |
| ATOM | 2211 | N | BSER | A | 649 | −17.399 | −35.647 | 25.884 | 0.26 | 12.05 | | | N |
| ANISOU | 2211 | N | BSER | A | 649 | 2127 | 912 | 1539 | 51 | −38 | −126 | | N |
| ATOM | 2212 | CA | BSER | A | 649 | −18.161 | −36.600 | 26.685 | 0.26 | 12.16 | | | C |
| ANISOU | 2212 | CA | BSER | A | 649 | 2191 | 889 | 1539 | 1 | −68 | −95 | | C |
| ATOM | 2213 | CB | BSER | A | 649 | −17.220 | −37.528 | 27.453 | 0.26 | 12.44 | | | C |
| ANISOU | 2213 | CB | BSER | A | 649 | 2181 | 874 | 1672 | −27 | −130 | 49 | | C |
| ATOM | 2214 | OG | BSER | A | 649 | −16.464 | −38.338 | 26.577 | 0.26 | 10.25 | | | O |
| ANISOU | 2214 | OG | BSER | A | 649 | 1841 | 470 | 1582 | 58 | −64 | 56 | | O |
| ATOM | 2215 | C | BSER | A | 649 | −19.089 | −35.882 | 27.652 | 0.26 | 12.27 | | | C |
| ANISOU | 2215 | C | BSER | A | 649 | 2218 | 993 | 1449 | −73 | −57 | −133 | | C |
| ATOM | 2216 | O | BSER | A | 649 | −20.254 | −36.264 | 27.806 | 0.26 | 13.38 | | | O |
| ANISOU | 2216 | O | BSER | A | 649 | 2355 | 1163 | 1564 | −104 | −39 | −156 | | O |
| ATOM | 2217 | N | CSER | A | 649 | −17.399 | −35.641 | 25.890 | 0.40 | 12.26 | | | N |
| ANISOU | 2217 | N | CSER | A | 649 | 2154 | 939 | 1565 | 51 | −38 | −126 | | N |
| ATOM | 2218 | CA | CSER | A | 649 | −18.162 | −36.602 | 26.678 | 0.40 | 12.25 | | | C |
| ANISOU | 2218 | CA | CSER | A | 649 | 2202 | 901 | 1551 | 1 | −68 | −95 | | C |
| ATOM | 2219 | CB | CSER | A | 649 | −17.219 | −37.554 | 27.417 | 0.40 | 12.18 | | | C |
| ANISOU | 2219 | CB | CSER | A | 649 | 2149 | 837 | 1644 | −24 | −128 | 47 | | C |
| ATOM | 2220 | OG | CSER | A | 649 | −16.547 | −36.886 | 28.472 | 0.40 | 14.10 | | | O |
| ANISOU | 2220 | OG | CSER | A | 649 | 2365 | 1178 | 1813 | −118 | −200 | 140 | | O |
| ATOM | 2221 | C | CSER | A | 649 | −19.082 | −35.894 | 27.663 | 0.40 | 12.04 | | | C |
| ANISOU | 2221 | C | CSER | A | 649 | 2190 | 965 | 1421 | −73 | −57 | −132 | | C |
| ATOM | 2222 | O | CSER | A | 649 | −20.239 | −36.291 | 27.833 | 0.40 | 13.77 | | | O |
| ANISOU | 2222 | O | CSER | A | 649 | 2405 | 1213 | 1613 | −106 | −41 | −153 | | O |
| ATOM | 2223 | N | ASN | A | 650 | −18.588 | −34.838 | 28.316 | 1.00 | 8.99 | | | N |
| ANISOU | 2223 | N | ASN | A | 650 | 1816 | 623 | 978 | −125 | −35 | −158 | | N |
| ATOM | 2224 | CA | ASN | A | 650 | −19.420 | −34.100 | 29.267 | 1.00 | 12.38 | | | C |
| ANISOU | 2224 | CA | ASN | A | 650 | 2266 | 1107 | 1330 | −228 | 63 | −259 | | C |
| ATOM | 2225 | CB | ASN | A | 650 | −18.595 | −33.026 | 29.976 | 1.00 | 14.20 | | | C |
| ANISOU | 2225 | CB | ASN | A | 650 | 2551 | 1370 | 1473 | −347 | 110 | −310 | | C |
| ATOM | 2226 | CG | ASN | A | 650 | −17.501 | −33.604 | 30.847 | 1.00 | 24.01 | | | C |
| ANISOU | 2226 | CG | ASN | A | 650 | 3813 | 2721 | 2589 | −528 | −21 | −159 | | C |
| ATOM | 2227 | OD1 | ASN | A | 650 | −17.558 | −34.761 | 31.259 | 1.00 | 23.17 | | | O |
| ANISOU | 2227 | OD1 | ASN | A | 650 | 3674 | 2659 | 2471 | −590 | −125 | −22 | | O |
| ATOM | 2228 | ND2 | ASN | A | 650 | −16.494 | −32.792 | 31.133 | 1.00 | 24.91 | | | N |
| ANISOU | 2228 | ND2 | ASN | A | 650 | 3964 | 2876 | 2626 | −633 | −36 | −144 | | N |
| ATOM | 2229 | C | ASN | A | 650 | −20.617 | −33.460 | 28.575 | 1.00 | 11.52 | | | C |
| ANISOU | 2229 | C | ASN | A | 650 | 2099 | 938 | 1341 | −118 | 159 | −338 | | C |
| ATOM | 2230 | O | ASN | A | 650 | −21.736 | −33.467 | 29.110 | 1.00 | 10.48 | | | O |
| ANISOU | 2230 | O | ASN | A | 650 | 1929 | 828 | 1227 | −160 | 253 | −398 | | O |
| ATOM | 2231 | N | ILE | A | 651 | −20.402 | −32.904 | 27.380 | 1.00 | 13.57 | | | N |
| ANISOU | 2231 | N | ILE | A | 651 | 2315 | 1141 | 1699 | −0 | 125 | −303 | | N |
| ATOM | 2232 | CA | ILE | A | 651 | −21.494 | −32.268 | 26.649 | 1.00 | 9.65 | | | C |
| ANISOU | 2232 | CA | ILE | A | 651 | 1728 | 582 | 1357 | 78 | 162 | −284 | | C |
| ATOM | 2233 | CB | ILE | A | 651 | −20.947 | −31.486 | 25.448 | 1.00 | 12.64 | | | C |
| ANISOU | 2233 | CB | ILE | A | 651 | 2089 | 911 | 1802 | 137 | 93 | −212 | | C |
| ATOM | 2234 | CG1 | ILE | A | 651 | −20.149 | −30.271 | 25.932 | 1.00 | 11.89 | | | C |
| ANISOU | 2234 | CG1 | ILE | A | 651 | 1976 | 807 | 1736 | 132 | 141 | −267 | | C |
| ATOM | 2235 | CD1 | ILE | A | 651 | −19.347 | −29.592 | 24.802 | 1.00 | 11.65 | | | C |
| ANISOU | 2235 | CD1 | ILE | A | 651 | 1938 | 756 | 1732 | 151 | 65 | −193 | | C |
| ATOM | 2236 | CG2 | ILE | A | 651 | −22.079 | −31.089 | 24.520 | 1.00 | 14.64 | | | C |
| ANISOU | 2236 | CG2 | ILE | A | 651 | 2202 | 1143 | 2218 | 170 | 43 | −76 | | C |
| ATOM | 2237 | C | ILE | A | 651 | −22.517 | −33.308 | 26.215 | 1.00 | 9.70 | | | C |
| ANISOU | 2237 | C | ILE | A | 651 | 1684 | 646 | 1355 | 62 | 100 | −211 | | C |
| ATOM | 2238 | O | ILE | A | 651 | −23.727 | −33.088 | 26.319 | 1.00 | 10.69 | | | O |
| ANISOU | 2238 | O | ILE | A | 651 | 1691 | 771 | 1599 | 77 | 151 | −183 | | O |
| ATOM | 2239 | N | LEU | A | 652 | −22.055 | −34.454 | 25.730 | 1.00 | 9.02 | | | N |
| ANISOU | 2239 | N | LEU | A | 652 | 1675 | 595 | 1157 | 22 | 13 | −180 | | N |
| ATOM | 2240 | CA | LEU | A | 652 | −22.989 | −35.507 | 25.346 | 1.00 | 13.38 | | | C |
| ANISOU | 2240 | CA | LEU | A | 652 | 2217 | 1196 | 1669 | −43 | −34 | −131 | | C |
| ATOM | 2241 | CB | LEU | A | 652 | −22.239 | −36.672 | 24.696 | 1.00 | 12.10 | | | C |
| ANISOU | 2241 | CB | LEU | A | 652 | 2166 | 1003 | 1428 | −95 | −65 | −148 | | C |
| ATOM | 2242 | CG | LEU | A | 652 | −21.705 | −36.392 | 23.292 | 1.00 | 12.67 | | | C |
| ANISOU | 2242 | CG | LEU | A | 652 | 2275 | 1070 | 1470 | −133 | −79 | −151 | | C |
| ATOM | 2243 | CD1 | LEU | A | 652 | −20.657 | −37.440 | 22.892 | 1.00 | 14.26 | | | C |
| ANISOU | 2243 | CD1 | LEU | A | 652 | 2540 | 1212 | 1667 | −154 | −6 | −226 | | C |
| ATOM | 2244 | CD2 | LEU | A | 652 | −22.848 | −36.340 | 22.274 | 1.00 | 10.12 | | | C |
| ANISOU | 2244 | CD2 | LEU | A | 652 | 1914 | 844 | 1086 | −269 | −155 | −57 | | C |
| ATOM | 2245 | C | LEU | A | 652 | −23.798 | −35.993 | 26.544 | 1.00 | 12.28 | | | C |
| ANISOU | 2245 | C | LEU | A | 652 | 2052 | 1102 | 1510 | −89 | 14 | −157 | | C |
| ATOM | 2246 | O | LEU | A | 652 | −24.993 | −36.278 | 26.412 | 1.00 | 12.21 | | | O |
| ANISOU | 2246 | O | LEU | A | 652 | 1966 | 1149 | 1525 | −128 | 6 | −106 | | O |
| ATOM | 2247 | N | ASP | A | 653 | −23.159 | −36.123 | 27.710 | 1.00 | 9.71 | | | N |
| ANISOU | 2247 | N | ASP | A | 653 | 1785 | 783 | 1122 | −130 | 49 | −209 | | N |
| ATOM | 2248 | CA | ASP | A | 653 | −23.883 | −36.522 | 28.918 | 1.00 | 13.84 | | | C |
| ANISOU | 2248 | CA | ASP | A | 653 | 2299 | 1385 | 1575 | −245 | 100 | −239 | | C |
| ATOM | 2249 | CB | ASP | A | 653 | −22.936 | −36.600 | 30.119 | 1.00 | 21.87 | | | C |
| ANISOU | 2249 | CB | ASP | A | 653 | 3394 | 2445 | 2469 | −374 | 90 | −246 | | C |
| ATOM | 2250 | CG | ASP | A | 653 | −22.048 | −37.827 | 30.105 | 1.00 | 39.74 | | | C |
| ANISOU | 2250 | CG | ASP | A | 653 | 5703 | 4677 | 4718 | −402 | −61 | −102 | | C |
| ATOM | 2251 | OD1 | ASP | A | 653 | −22.374 | −38.809 | 29.402 | 1.00 | 49.87 | | | O |
| ANISOU | 2251 | OD1 | ASP | A | 653 | 6990 | 5898 | 6060 | −356 | −110 | −55 | | O |
| ATOM | 2252 | OD2 | ASP | A | 653 | −21.025 | −37.810 | 30.826 | 1.00 | 42.35 | | | O |
| ANISOU | 2252 | OD2 | ASP | A | 653 | 6054 | 5036 | 5001 | −494 | −124 | −22 | | O |
| ATOM | 2253 | C | ASP | A | 653 | −25.004 | −35.549 | 29.242 | 1.00 | 16.14 | | | C |
| ANISOU | 2253 | C | ASP | A | 653 | 2473 | 1688 | 1970 | −229 | 257 | −319 | | C |
| ATOM | 2254 | O | ASP | A | 653 | −26.107 | −35.960 | 29.626 | 1.00 | 12.52 | | | O |
| ANISOU | 2254 | O | ASP | A | 653 | 1945 | 1297 | 1514 | −291 | 307 | −322 | | O |
| ATOM | 2255 | N | VAL | A | 654 | −24.727 | −34.249 | 29.140 | 1.00 | 11.98 | | | N |
| ANISOU | 2255 | N | VAL | A | 654 | 1909 | 1076 | 1568 | −150 | 362 | −387 | | N |
| ATOM | 2256 | CA | VAL | A | 654 | −25.741 | −33.254 | 29.461 | 1.00 | 13.72 | | | C |
| ANISOU | 2256 | CA | VAL | A | 654 | 1984 | 1231 | 1999 | −108 | 572 | −472 | | C |
| ATOM | 2257 | CB | VAL | A | 654 | −25.105 | −31.858 | 29.583 | 1.00 | 18.39 | | | C |
| ANISOU | 2257 | CB | VAL | A | 654 | 2591 | 1681 | 2715 | −58 | 714 | −581 | | C |
| ATOM | 2258 | CG1 | VAL | A | 654 | −26.184 | −30.819 | 29.809 | 1.00 | 20.76 | | | C |
| ANISOU | 2258 | CG1 | VAL | A | 654 | 2704 | 1831 | 3354 | 20 | 979 | −667 | | C |
| ATOM | 2259 | CG2 | VAL | A | 654 | −24.075 | −31.833 | 30.704 | 1.00 | 19.96 | | | C |
| ANISOU | 2259 | CG2 | VAL | A | 654 | 2972 | 1958 | 2654 | −250 | 757 | −710 | | C |
| ATOM | 2260 | C | VAL | A | 654 | −26.847 | −33.269 | 28.422 | 1.00 | 14.26 | | | C |
| ANISOU | 2260 | C | VAL | A | 654 | 1855 | 1281 | 2281 | 3 | 504 | −303 | | C |
| ATOM | 2261 | O | VAL | A | 654 | −28.027 | −33.099 | 28.749 | 1.00 | 15.97 | | | O |
| ANISOU | 2261 | O | VAL | A | 654 | 1898 | 1493 | 2677 | 15 | 639 | −304 | | O |
| ATOM | 2262 | N | MET | A | 65 5 | −26.493 | −33.456 | 27.150 | 1.00 | 13.24 | | | N |
| ANISOU | 2262 | N | MET | A | 65 5 | 1737 | 1158 | 2135 | 49 | 301 | −141 | | N |
| ATOM | 2263 | CA | MET | A | 65 5 | −27.534 | −33.598 | 26.139 | 1.00 | 14.15 | | | C |
| ANISOU | 2263 | CA | MET | A | 65 5 | 1680 | 1319 | 2377 | 54 | 182 | 73 | | C |
| ATOM | 2264 | CB | MET | A | 65 5 | −26.913 | −33.819 | 24.764 | 1.00 | 13.30 | | | C |
| ANISOU | 2264 | CB | MET | A | 65 5 | 1654 | 1252 | 2148 | 2 | −25 | 205 | | C |
| ATOM | 2265 | CG | MET | A | 65 5 | −26.422 | −32.581 | 24.114 | 1.00 | 28.12 | | | C |
| ANISOU | 2265 | CG | MET | A | 65 5 | 3468 | 3032 | 4186 | 80 | −51 | 286 | | C |
| ATOM | 2266 | SD | MET | A | 65 5 | −25.867 | −32.948 | 22.439 | 1.00 | 28.57 | | | S |
| ANISOU | 2266 | SD | MET | A | 65 5 | 3622 | 3200 | 4032 | −86 | −279 | 437 | | S |
| ATOM | 2267 | CE | MET | A | 65 5 | −25.345 | −31.312 | 21.945 | 1.00 | 32.39 | | | C |
| ANISOU | 2267 | CE | MET | A | 65 5 | 4005 | 3561 | 4739 | 6 | −306 | 551 | | C |
| ATOM | 2268 | C | MET | A | 65 5 | −28.469 | −34.753 | 26.468 | 1.00 | 15.75 | | | C |
| ANISOU | 2268 | C | MET | A | 65 5 | 1862 | 1651 | 2469 | −54 | 155 | 100 | | C |
| ATOM | 2269 | O | MET | A | 65 5 | −29.691 | −34.647 | 26.298 | 1.00 | 18.10 | | | O |
| ANISOU | 2269 | O | MET | A | 65 5 | 1943 | 1989 | 2946 | −54 | 161 | 239 | | O |
| ATOM | 2270 | N | ASP | A | 65 6 | −27.912 | −35.879 | 26.908 | 1.00 | 13.20 | | | N |
| ANISOU | 2270 | N | ASP | A | 65 6 | 1742 | 1388 | 1886 | −153 | 114 | 5 | | N |
| ATOM | 2271 | CA | ASP | A | 65 6 | −28.756 | −37.004 | 27.277 | 1.00 | 15.35 | | | C |
| ANISOU | 2271 | CA | ASP | A | 65 6 | 2016 | 1770 | 2046 | −275 | 84 | 32 | | C |
| ATOM | 2272 | CB | ASP | A | 65 6 | −27.902 | −38.238 | 27.576 | 1.00 | 17.09 | | | C |
| ANISOU | 2272 | CB | ASP | A | 65 6 | 2460 | 1991 | 2043 | −369 | 9 | −26 | | C |
| ATOM | 2273 | CG | ASP | A | 65 6 | −27.345 | −38.890 | 26.315 | 1.00 | 19.68 | | | C |
| ANISOU | 2273 | CG | ASP | A | 65 6 | 2905 | 2277 | 2295 | −404 | −112 | 26 | | C |
| ATOM | 2274 | OD1 | ASP | A | 65 6 | −27.813 | −38.551 | 25.203 | 1.00 | 18.84 | | | O |
| ANISOU | 2274 | OD1 | ASP | A | 65 6 | 2730 | 2213 | 2217 | −435 | −178 | 126 | | O |
| ATOM | 2275 | OD2 | ASP | A | 65 6 | −26.447 | −39.752 | 26.444 | 1.00 | 18.24 | | | O |
| ANISOU | 2275 | OD2 | ASP | A | 65 6 | 2874 | 2017 | 2040 | −431 | −130 | −23 | | O |
| ATOM | 2276 | C | ASP | A | 65 6 | −29.622 | −36.662 | 28.479 | 1.00 | 18.81 | | | C |
| ANISOU | 2276 | C | ASP | A | 65 6 | 2324 | 2241 | 2580 | −289 | 276 | −58 | | C |
| ATOM | 2277 | O | ASP | A | 65 6 | −30.816 | −36.979 | 28.503 | 1.00 | 19.05 | | | O |
| ANISOU | 2277 | O | ASP | A | 65 6 | 2204 | 2359 | 2676 | −340 | 288 | 26 | | O |
| ATOM | 2278 | N | GLU | A | 657 | −29.037 | −36.007 | 29.484 | 1.00 | 15.22 | | | N |
| ANISOU | 2278 | N | GLU | A | 657 | 1929 | 1732 | 2122 | −284 | 448 | −238 | | N |
| ATOM | 2279 | CA | GLU | A | 657 | −29.766 | −35.743 | 30.722 | 1.00 | 25.99 | | | C |
| ANISOU | 2279 | CA | GLU | A | 657 | 3218 | 3139 | 3518 | −377 | 693 | −394 | | C |
| ATOM | 2280 | CB | GLU | A | 657 | −28.788 | −35.378 | 31.843 | 1.00 | 31.13 | | | C |
| ANISOU | 2280 | CB | GLU | A | 657 | 4049 | 3787 | 3992 | −508 | 820 | −594 | | C |
| ATOM | 2281 | CG | GLU | A | 657 | −27.767 | −36.478 | 32.135 | 1.00 | 45.79 | | | C |
| ANISOU | 2281 | CG | GLU | A | 657 | 6118 | 5737 | 5544 | −650 | 594 | −517 | | C |
| ATOM | 2282 | CD | GLU | A | 657 | −26.473 | −35.943 | 32.719 | 1.00 | 64.38 | | | C |
| ANISOU | 2282 | CD | GLU | A | 657 | 8616 | 8073 | 7771 | −738 | 610 | −594 | | C |
| ATOM | 2283 | OE1 | GLU | A | 657 | −26.451 | −34.760 | 33.115 | 1.00 | 70.30 | | | O |
| ANISOU | 2283 | OE1 | GLU | A | 657 | 9353 | 8762 | 8598 | −753 | 838 | −771 | | O |
| ATOM | 2284 | OE2 | GLU | A | 657 | −25.475 | −36.698 | 32.769 | 1.00 | 69.69 | | | O |
| ANISOU | 2284 | OE2 | GLU | A | 657 | 9400 | 8778 | 8299 | −801 | 403 | −465 | | O |
| ATOM | 2285 | C | GLU | A | 657 | −30.805 | −34.647 | 30.542 | 1.00 | 23.85 | | | C |
| ANISOU | 2285 | C | GLU | A | 657 | 2664 | 2763 | 3637 | −239 | 906 | −394 | | C |
| ATOM | 2286 | O | GLU | A | 657 | −31.828 | −34.651 | 31.233 | 1.00 | 22.50 | | | O |
| ANISOU | 2286 | O | GLU | A | 657 | 2344 | 2634 | 3569 | −299 | 1111 | −472 | | O |
| ATOM | 2287 | N | GLU | A | 658 | −30.579 | −33.721 | 29.615 | 1.00 | 23.54 | | | N |
| ANISOU | 2287 | N | GLU | A | 658 | 2518 | 2574 | 3853 | −61 | 865 | −285 | | N |
| ATOM | 2288 | CA | GLU | A | 658 | −31.531 | −32.641 | 29.386 | 1.00 | 37.83 | | | C |
| ANISOU | 2288 | CA | GLU | A | 658 | 4003 | 4227 | 6143 | 95 | 1049 | −213 | | C |
| ATOM | 2289 | CB | GLU | A | 658 | −30.813 | −31.289 | 29.349 | 1.00 | 44.04 | | | C |
| ANISOU | 2289 | CB | GLU | A | 658 | 4795 | 4776 | 7161 | 221 | 1202 | −315 | | C |
| ATOM | 2290 | CG | GLU | A | 658 | −30.095 | −30.916 | 30.649 | 1.00 | 45.44 | | | C |
| ANISOU | 2290 | CG | GLU | A | 658 | 5198 | 4908 | 7159 | 91 | 1488 | −686 | | C |
| ATOM | 2291 | CD | GLU | A | 658 | −29.328 | −29.586 | 30.558 | 1.00 | 49.40 | | | C |
| ANISOU | 2291 | CD | GLU | A | 658 | 5778 | 5239 | 7753 | 121 | 1501 | −709 | | C |
| ATOM | 2292 | OE1 | GLU | A | 658 | −29.313 | −28.952 | 29.475 | 1.00 | 56.62 | | | O |
| ANISOU | 2292 | OE1 | GLU | A | 658 | 6554 | 6039 | 8918 | 281 | 1361 | −487 | | O |
| ATOM | 2293 | OE2 | GLU | A | 658 | −28.732 | −29.175 | 31.576 | 1.00 | 35.16 | | | O |
| ANISOU | 2293 | OE2 | GLU | A | 658 | 4158 | 3449 | 5751 | −60 | 1636 | −919 | | O |
| ATOM | 2294 | C | GLU | A | 658 | −32.309 | −32.875 | 28.091 | 1.00 | 45.63 | | | C |
| ANISOU | 2294 | C | GLU | A | 658 | 4762 | 5275 | 7300 | 152 | 783 | 161 | | C |
| ATOM | 2295 | O | GLU | A | 658 | −32.156 | −32.141 | 27.116 | 1.00 | 48.75 | | | O |
| ANISOU | 2295 | O | GLU | A | 658 | 5037 | 5569 | 7918 | 256 | 656 | 371 | | O |
| HETATM | 2296 | C01 | LIG | A | 1 | −18.924 | −12.046 | 13.195 | 1.00 | 15.02 | | A | C |
| HETATM | 2297 | C02 | LIG | A | 1 | −17.769 | −12.009 | 12.218 | 1.00 | 16.76 | | A | C |
| HETATM | 2298 | C03 | LIG | A | 1 | −18.026 | −11.155 | 10.980 | 1.00 | 22.91 | | A | C |
| HETATM | 2299 | O04 | LIG | A | 1 | −18.759 | −10.183 | 11.030 | 1.00 | 24.67 | | A | O |
| HETATM | 2300 | N05 | LIG | A | 1 | −17.447 | −11.496 | 9.784 | 1.00 | 22.08 | | A | N |
| HETATM | 2301 | C06 | LIG | A | 1 | −17.601 | −10.714 | 8.585 | 1.00 | 24.36 | | A | C |
| HETATM | 2302 | C07 | LIG | A | 1 | −16.279 | −10.534 | 7.843 | 1.00 | 28.76 | | A | C |
| HETATM | 2303 | C08 | LIG | A | 1 | −16.672 | −10.244 | 6.394 | 1.00 | 23.13 | | A | C |
| HETATM | 2304 | C09 | LIG | A | 1 | −18.174 | −10.405 | 6.315 | 1.00 | 27.01 | | A | C |
| HETATM | 2305 | C10 | LIG | A | 1 | −18.448 | −11.337 | 7.473 | 1.00 | 22.92 | | A | C |
| HETATM | 2306 | N11 | LIG | A | 1 | −18.040 | −12.675 | 7.088 | 1.00 | 13.11 | | A | N |
| HETATM | 2307 | C12 | LIG | A | 1 | −18.809 | −13.738 | 6.661 | 1.00 | 17.10 | | A | C |
| HETATM | 2308 | O13 | LIG | A | 1 | −20.039 | −13.565 | 6.614 | 1.00 | 13.18 | | A | O |
| HETATM | 2309 | C14 | LIG | A | 1 | −18.360 | −15.051 | 6.250 | 1.00 | 9.60 | | A | C |
| HETATM | 2310 | S15 | LIG | A | 1 | −19.413 | −16.333 | 5.605 | 1.00 | 11.77 | | A | S |
| HETATM | 2311 | C16 | LIG | A | 1 | −18.065 | −17.480 | 5.367 | 1.00 | 4.84 | | A | C |
| HETATM | 2312 | N17 | LIG | A | 1 | −18.129 | −18.727 | 4.877 | 1.00 | 8.39 | | A | N |
| HETATM | 2313 | C18 | LIG | A | 1 | −16.965 | −19.394 | 4.811 | 1.00 | 11.17 | | A | C |
| HETATM | 2314 | C19 | LIG | A | 1 | −15.722 | −18.876 | 5.195 | 1.00 | 9.38 | | A | C |
| HETATM | 2315 | C20 | LIG | A | 1 | −15.668 | −17.584 | 5.697 | 1.00 | 5.96 | | A | C |
| HETATM | 2316 | C21 | LIG | A | 1 | −16.876 | −16.877 | 5.778 | 1.00 | 8.23 | | A | C |
| HETATM | 2317 | C22 | LIG | A | 1 | −17.088 | −15.536 | 6.259 | 1.00 | 10.04 | | A | C |
| HETATM | 2318 | N23 | LIG | A | 1 | −15.874 | −14.988 | 6.632 | 1.00 | 11.10 | | A | N |
| HETATM | 2319 | C24 | LIG | A | 1 | −14.617 | −15.632 | 6.599 | 1.00 | 14.44 | | A | C |
| HETATM | 2320 | O25 | LIG | A | 1 | −13.632 | −15.029 | 6.975 | 1.00 | 10.44 | | A | O |
| HETATM | 2321 | N26 | LIG | A | 1 | −14.501 | −16.956 | 6.088 | 1.00 | 8.76 | | A | N |
| HETATM | 2322 | C27 | LIG | A | 1 | −13.275 | −17.563 | 6.123 | 1.00 | 14.75 | | A | C |
| HETATM | 2323 | C28 | LIG | A | 1 | −12.777 | −18.003 | 7.378 | 1.00 | 12.74 | | A | C |
| HETATM | 2324 | N29 | LIG | A | 1 | −11.626 | −18.639 | 7.595 | 1.00 | 14.25 | | A | N |
| HETATM | 2325 | C30 | LIG | A | 1 | −10.818 | −18.902 | 6.551 | 1.00 | 16.18 | | A | C |
| HETATM | 2326 | C31 | LIG | A | 1 | −11.266 | −18.516 | 5.269 | 1.00 | 9.86 | | A | C |
| HETATM | 2327 | C32 | LIG | A | 1 | −12.461 | −17.858 | 5.036 | 1.00 | 9.68 | | A | C |
| HETATM | 2328 | C33 | LIG | A | 1 | −12.917 | −17.449 | 3.660 | 1.00 | 13.02 | | A | C |
| HETATM | 2329 | C34 | LIG | A | 1 | −9.522 | −19.663 | 6.816 | 1.00 | 14.99 | | A | C |
| HETATM | 2330 | C35 | LIG | A | 1 | −9.512 | −21.180 | 6.396 | 1.00 | 16.68 | | A | C |
| HETATM | 2331 | C36 | LIG | A | 1 | −10.689 | −22.003 | 6.970 | 1.00 | 15.58 | | A | C |
| HETATM | 2332 | C37 | LIG | A | 1 | −8.238 | −21.927 | 6.692 | 1.00 | 19.43 | | A | C |
| HETATM | 2333 | H061 | LIG | A | 1 | −17.886 | −9.847 | 8.806 | 1.00 | 29.24 | | A | H |
| HETATM | 2334 | H101 | LIG | A | 1 | −19.359 | −11.245 | 7.680 | 1.00 | 27.51 | | A | H |
| HETATM | 2335 | H021 | LIG | A | 1 | −17.004 | −11.723 | 12.681 | 1.00 | 20.11 | | A | H |
| HETATM | 2336 | H022 | LIG | A | 1 | −17.588 | −12.896 | 11.968 | 1.00 | 20.11 | | A | H |
| HETATM | 2337 | H051 | LIG | A | 1 | −16.904 | −12.170 | 9.717 | 1.00 | 26.50 | | A | H |
| HETATM | 2338 | H071 | LIG | A | 1 | −15.762 | −9.852 | 8.230 | 1.00 | 34.51 | | A | H |
| HETATM | 2339 | H072 | LIG | A | 1 | −15.782 | −11.322 | 7.955 | 1.00 | 34.51 | | A | H |
| HETATM | 2340 | H081 | LIG | A | 1 | −16.393 | −9.398 | 6.097 | 1.00 | 27.76 | | A | H |
| HETATM | 2341 | H082 | LIG | A | 1 | −16.258 | −10.852 | 5.810 | 1.00 | 27.76 | | A | H |
| HETATM | 2342 | H091 | LIG | A | 1 | −18.507 | −10.772 | 5.518 | 1.00 | 32.41 | | A | H |
| HETATM | 2343 | H092 | LIG | A | 1 | −18.637 | −9.589 | 6.354 | 1.00 | 32.41 | | A | H |
| HETATM | 2344 | H111 | LIG | A | 1 | −17.175 | −12.743 | 7.124 | 1.00 | 15.73 | | A | H |
| HETATM | 2345 | H181 | LIG | A | 1 | −16.989 | −20.263 | 4.483 | 1.00 | 13.41 | | A | H |
| HETATM | 2346 | H191 | LIG | A | 1 | −14.983 | −19.432 | 5.108 | 1.00 | 11.26 | | A | H |
| HETATM | 2347 | H231 | LIG | A | 1 | −15.850 | −14.183 | 6.956 | 1.00 | 13.32 | | A | H |
| HETATM | 2348 | H281 | LIG | A | 1 | −13.235 | −17.872 | 8.175 | 1.00 | 15.29 | | A | H |
| HETATM | 2349 | H311 | LIG | A | 1 | −10.705 | −18.720 | 4.558 | 1.00 | 11.83 | | A | H |
| HETATM | 2350 | H331 | LIG | A | 1 | −12.240 | −17.453 | 2.965 | 1.00 | 15.63 | | A | H |
| HETATM | 2351 | H332 | LIG | A | 1 | −13.282 | −16.551 | 3.619 | 1.00 | 15.63 | | A | H |
| HETATM | 2352 | H333 | LIG | A | 1 | −13.625 | −18.004 | 3.300 | 1.00 | 15.63 | | A | H |
| HETATM | 2353 | H341 | LIG | A | 1 | −9.279 | −19.602 | 7.754 | 1.00 | 17.99 | | A | H |
| HETATM | 2354 | H342 | LIG | A | 1 | −8.765 | −19.235 | 6.386 | 1.00 | 17.99 | | A | H |
| HETATM | 2355 | H351 | LIG | A | 1 | −9.590 | −21.188 | 5.429 | 1.00 | 20.02 | | A | H |
| HETATM | 2356 | H361 | LIG | A | 1 | −11.389 | −22.238 | 6.342 | 1.00 | 18.70 | | A | H |
| HETATM | 2357 | H362 | LIG | A | 1 | −10.444 | −22.850 | 7.374 | 1.00 | 18.70 | | A | H |
| HETATM | 2358 | H363 | LIG | A | 1 | −11.174 | −21.536 | 7.668 | 1.00 | 18.70 | | A | H |
| HETATM | 2359 | H371 | LIG | A | 1 | −7.484 | −21.667 | 6.140 | 1.00 | 23.32 | | A | H |
| HETATM | 2360 | H372 | LIG | A | 1 | −8.287 | −22.882 | 6.528 | 1.00 | 23.32 | | A | H |
| HETATM | 2361 | H373 | LIG | A | 1 | −7.903 | −21.877 | 7.601 | 1.00 | 23.32 | | A | H |
| TER | | | | | | | | | | | | | |
| HETATM | 2362 | O | HOH | S | 1 | −28.356 | −16.157 | 28.996 | 1.00 | 8.18 | | | O |
| HETATM | 2363 | O | HOH | S | 2 | −23.778 | −22.748 | 3.921 | 1.00 | 10.79 | | | O |
| HETATM | 2364 | O | HOH | S | 3 | −11.246 | −14.935 | 29.778 | 1.00 | 11.84 | | | O |
| HETATM | 2365 | O | HOH | S | 4 | −13.850 | −16.632 | 33.319 | 1.00 | 7.93 | | | O |
| HETATM | 2366 | O | HOH | S | 5 | −30.782 | −15.074 | 19.843 | 1.00 | 13.54 | | | O |
| HETATM | 2367 | O | HOH | S | 6 | −9.169 | −13.455 | 30.033 | 1.00 | 13.95 | | | O |
| HETATM | 2368 | O | HOH | S | 7 | −16.575 | −29.312 | 6.763 | 1.00 | 9.52 | | | O |
| HETATM | 2369 | O | HOH | S | 8 | −17.086 | −20.469 | −11.337 | 1.00 | 12.41 | | | O |
| HETATM | 2370 | O | HOH | S | 9 | −23.295 | −13.941 | 33.140 | 1.00 | 15.94 | | | O |
| HETATM | 2371 | O | HOH | S | 10 | −29.099 | −24.864 | 29.749 | 1.00 | 15.26 | | | O |
| HETATM | 2372 | O | HOH | S | 11 | −16.351 | −13.418 | 19.557 | 1.00 | 10.09 | | | O |
| HETATM | 2373 | O | HOH | S | 12 | −8.004 | −20.261 | 21.120 | 1.00 | 9.98 | | | O |
| HETATM | 2374 | O | HOH | S | 13 | −22.199 | −7.109 | 25.534 | 1.00 | 17.85 | | | O |
| HETATM | 2375 | O | HOH | S | 14 | −15.020 | −18.628 | −13.422 | 1.00 | 12.35 | | | O |
| HETATM | 2376 | O | HOH | S | 15 | −20.203 | −19.375 | −13.208 | 1.00 | 10.68 | | | O |
| HETATM | 2377 | O | HOH | S | 16 | −9.021 | −24.193 | −9.542 | 1.00 | 14.56 | | | O |
| HETATM | 2378 | O | HOH | S | 17 | −16.539 | −13.243 | −11.273 | 1.00 | 16.03 | | | O |
| HETATM | 2379 | O | HOH | S | 18 | −25.750 | −26.372 | 15.537 | 1.00 | 15.99 | | | O |
| HETATM | 2380 | O | HOH | S | 19 | −10.411 | −8.868 | 24.456 | 1.00 | 11.18 | | | O |
| HETATM | 2381 | O | HOH | S | 20 | −16.633 | −26.784 | 36.955 | 1.00 | 16.80 | | | O |
| HETATM | 2382 | O | HOH | S | 21 | −15.797 | −10.718 | 19.501 | 1.00 | 13.61 | | | O |
| HETATM | 2383 | O | HOH | S | 22 | 7.354 | −14.909 | 28.387 | 1.00 | 16.55 | | | O |
| HETATM | 2384 | O | HOH | S | 23 | −32.676 | −16.492 | 21.201 | 1.00 | 15.19 | | | O |
| HETATM | 2385 | O | HOH | S | 24 | −9.630 | −16.126 | 21.211 | 1.00 | 14.08 | | | O |
| HETATM | 2386 | O | HOH | S | 25 | −5.269 | −18.272 | 15.235 | 1.00 | 12.57 | | | O |
| HETATM | 2387 | O | HOH | S | 26 | −30.155 | −37.946 | 23.629 | 1.00 | 11.83 | | | O |
| HETATM | 2388 | O | HOH | S | 27 | −20.493 | −13.197 | 33.592 | 1.00 | 16.15 | | | O |
| HETATM | 2389 | O | HOH | S | 28 | −4.544 | −20.872 | 22.310 | 1.00 | 12.82 | | | O |
| HETATM | 2390 | O | HOH | S | 29 | −28.435 | −26.509 | 27.632 | 1.00 | 13.73 | | | O |
| HETATM | 2391 | O | HOH | S | 30 | −1.502 | −22.551 | 28.061 | 1.00 | 13.99 | | | O |
| HETATM | 2392 | O | HOH | S | 31 | −18.834 | −11.753 | 22.350 | 1.00 | 14.19 | | | O |
| HETATM | 2393 | O | HOH | S | 32 | −6.523 | −17.239 | 37.857 | 1.00 | 14.17 | | | O |
| HETATM | 2394 | O | HOH | S | 33 | −17.497 | −9.799 | 21.602 | 1.00 | 14.80 | | | O |
| HETATM | 2395 | O | HOH | S | 34 | −30.743 | −21.299 | 6.725 | 1.00 | 15.00 | | | O |
| HETATM | 2396 | O | HOH | S | 35 | 2.936 | −23.248 | 14.848 | 1.00 | 16.88 | | | O |
| HETATM | 2397 | O | HOH | S | 36 | −3.355 | −21.500 | 10.109 | 1.00 | 16.79 | | | O |
| HETATM | 2398 | O | HOH | S | 37 | −8.713 | −34.221 | 23.064 | 1.00 | 16.35 | | | O |
| HETATM | 2399 | O | HOH | S | 38 | −12.762 | −29.598 | 7.123 | 1.00 | 13.28 | | | O |
| HETATM | 2400 | O | HOH | S | 39 | 4.882 | −21.462 | 7.677 | 1.00 | 18.73 | | | O |
| HETATM | 2401 | O | HOH | S | 40 | −8.949 | −21.929 | 39.341 | 1.00 | 15.06 | | | O |
| HETATM | 2402 | O | HOH | S | 41 | −1.760 | −23.124 | 15.218 | 1.00 | 23.36 | | | O |
| HETATM | 2403 | O | HOH | S | 42 | −14.039 | −34.690 | 16.861 | 1.00 | 21.22 | | | O |
| HETATM | 2404 | O | HOH | S | 43 | −34.131 | −21.131 | 16.025 | 1.00 | 17.12 | | | O |
| HETATM | 2405 | O | HOH | S | 44 | −11.440 | −21.224 | −15.718 | 1.00 | 16.51 | | | O |
| HETATM | 2406 | O | HOH | S | 45 | −14.986 | −30.791 | 32.209 | 1.00 | 20.30 | | | O |
| HETATM | 2407 | O | HOH | S | 46 | −23.139 | −36.689 | 18.214 | 1.00 | 18.93 | | | O |
| HETATM | 2408 | O | HOH | S | 47 | −8.996 | 0.163 | 32.288 | 1.00 | 24.93 | | | O |
| HETATM | 2409 | O | HOH | S | 48 | −0.681 | −9.520 | 3.274 | 1.00 | 20.52 | | | O |
| HETATM | 2410 | O | HOH | S | 49 | −31.482 | −39.504 | 27.221 | 1.00 | 21.64 | | | O |
| HETATM | 2411 | O | HOH | S | 50 | −24.230 | −13.858 | 30.613 | 1.00 | 16.97 | | | O |
| HETATM | 2412 | O | HOH | S | 51 | 3.183 | −24.405 | 25.973 | 1.00 | 14.03 | | | O |
| HETATM | 2413 | O | HOH | S | 52 | 6.215 | −14.929 | 5.107 | 1.00 | 22.39 | | | O |
| HETATM | 2414 | O | HOH | S | 53 | −27.180 | −13.618 | 30.972 | 1.00 | 22.94 | | | O |
| HETATM | 2415 | O | HOH | S | 54 | −24.285 | −14.310 | 11.195 | 1.00 | 10.27 | | | O |
| HETATM | 2416 | O | HOH | S | 55 | −28.243 | −28.832 | 35.506 | 1.00 | 17.51 | | | O |
| HETATM | 2417 | O | HOH | S | 56 | −8.426 | −34.997 | 20.765 | 1.00 | 19.00 | | | O |
| HETATM | 2418 | O | HOH | S | 57 | −22.465 | −7.925 | −3.470 | 1.00 | 24.08 | | | O |
| HETATM | 2419 | O | HOH | S | 58 | −5.088 | −32.961 | 30.127 | 1.00 | 20.57 | | | O |
| HETATM | 2420 | O | HOH | S | 59 | −6.451 | −26.631 | −4.188 | 1.00 | 16.53 | | | O |
| HETATM | 2421 | O | HOH | S | 61 | −19.493 | −10.497 | 31.913 | 1.00 | 22.56 | | | O |
| HETATM | 2422 | O | HOH | S | 62 | −12.608 | −20.812 | 40.634 | 1.00 | 20.25 | | | O |
| HETATM | 2423 | O | HOH | S | 63 | −19.580 | −24.679 | 0.279 | 1.00 | 25.89 | | | O |
| HETATM | 2424 | O | HOH | S | 64 | −0.952 | −23.187 | 34.417 | 1.00 | 23.74 | | | O |
| HETATM | 2425 | O | HOH | S | 65 | −29.952 | −26.237 | 36.363 | 1.00 | 25.36 | | | O |
| HETATM | 2426 | O | HOH | S | 66 | −27.945 | −25.866 | 17.124 | 1.00 | 15.95 | | | O |
| HETATM | 2427 | O | HOH | S | 67 | −12.252 | −23.624 | 37.104 | 1.00 | 15.26 | | | O |
| HETATM | 2428 | O | HOH | S | 68 | −17.538 | −11.829 | 36.258 | 1.00 | 21.45 | | | O |
| HETATM | 2429 | O | HOH | S | 69 | −22.701 | −20.838 | −12.014 | 1.00 | 19.68 | | | O |
| HETATM | 2430 | O | HOH | S | 70 | −21.272 | −12.944 | 9.004 | 1.00 | 17.18 | | | O |
| HETATM | 2431 | O | HOH | S | 71 | −26.709 | −9.788 | 14.793 | 1.00 | 20.44 | | | O |
| HETATM | 2432 | O | HOH | S | 72 | −8.634 | −37.025 | 27.174 | 1.00 | 21.26 | | | O |
| HETATM | 2433 | O | HOH | S | 73 | −4.918 | −5.503 | 28.298 | 1.00 | 29.85 | | | O |
| HETATM | 2434 | O | HOH | S | 75 | −33.507 | −10.349 | 15.555 | 1.00 | 21.72 | | | O |
| HETATM | 2435 | O | HOH | S | 76 | −25.802 | −13.529 | 34.009 | 1.00 | 16.65 | | | O |
| HETATM | 2436 | O | HOH | S | 77 | −10.102 | −24.391 | −15.202 | 1.00 | 23.33 | | | O |
| HETATM | 2437 | O | HOH | S | 78 | −34.711 | −14.644 | 21.542 | 1.00 | 22.01 | | | O |
| HETATM | 2438 | O | HOH | S | 79 | −25.104 | −29.820 | 14.517 | 1.00 | 21.70 | | | O |
| HETATM | 2439 | O | HOH | S | 80 | −25.360 | −22.746 | 1.620 | 1.00 | 17.32 | | | O |
| HETATM | 2440 | O | HOH | S | 81 | −3.345 | −16.964 | −8.402 | 1.00 | 23.59 | | | O |
| HETATM | 2441 | O | HOH | S | 82 | 0.712 | −23.381 | 32.341 | 1.00 | 21.85 | | | O |
| HETATM | 2442 | O | HOH | S | 83 | −0.358 | −25.993 | 16.210 | 1.00 | 28.08 | | | O |
| HETATM | 2443 | O | HOH | S | 84 | 0.023 | −24.155 | 29.771 | 1.00 | 19.35 | | | O |
| HETATM | 2444 | O | HOH | S | 85 | −28.127 | −23.323 | 14.642 | 1.00 | 21.19 | | | O |
| HETATM | 2445 | O | HOH | S | 86 | −5.895 | −30.566 | 26.163 | 1.00 | 21.91 | | | O |
| HETATM | 2446 | O | HOH | S | 87 | −32.934 | −18.076 | 23.294 | 1.00 | 16.85 | | | O |
| HETATM | 2447 | O | HOH | S | 88 | −19.442 | −36.494 | 18.804 | 1.00 | 21.42 | | | O |
| HETATM | 2448 | O | HOH | S | 89 | −0.586 | −26.368 | 34.979 | 1.00 | 25.53 | | | O |
| HETATM | 2449 | O | HOH | S | 90 | −7.077 | −0.786 | 34.266 | 1.00 | 32.74 | | | O |
| HETATM | 2450 | O | HOH | S | 91 | −7.881 | −14.579 | 14.271 | 1.00 | 22.48 | | | O |
| HETATM | 2451 | O | HOH | S | 92 | −13.148 | −12.347 | 7.714 | 1.00 | 27.05 | | | O |
| HETATM | 2452 | O | HOH | S | 93 | −16.246 | −8.883 | 17.483 | 1.00 | 21.49 | | | O |
| HETATM | 2453 | O | HOH | S | 94 | −11.387 | −21.174 | 38.198 | 1.00 | 21.72 | | | O |
| HETATM | 2454 | O | HOH | S | 95 | −4.149 | −5.328 | 33.878 | 1.00 | 23.15 | | | O |
| HETATM | 2455 | O | HOH | S | 96 | −2.058 | −7.581 | 9.923 | 1.00 | 24.92 | | | O |
| HETATM | 2456 | O | HOH | S | 97 | −17.414 | −30.780 | 34.002 | 1.00 | 31.10 | | | O |
| HETATM | 2457 | O | HOH | S | 98 | −22.726 | −25.357 | 38.124 | 1.00 | 22.44 | | | O |
| HETATM | 2458 | O | HOH | S | 99 | −22.168 | −26.929 | 5.149 | 1.00 | 24.31 | | | O |
| HETATM | 2459 | O | HOH | S | 100 | −38.410 | −28.221 | 24.129 | 1.00 | 28.39 | | | O |
| HETATM | 2460 | O | HOH | S | 101 | −24.937 | −27.138 | 3.577 | 1.00 | 30.94 | | | O |
| HETATM | 2461 | O | HOH | S | 102 | −7.543 | −17.123 | 22.848 | 1.00 | 27.87 | | | O |
| HETATM | 2462 | O | HOH | S | 103 | −5.152 | −15.568 | 23.248 | 1.00 | 19.74 | | | O |
| HETATM | 2463 | O | HOH | S | 104 | −6.777 | −36.944 | 29.331 | 1.00 | 31.74 | | | O |
| HETATM | 2464 | O | HOH | S | 105 | −6.483 | −27.577 | 19.701 | 1.00 | 22.68 | | | O |
| HETATM | 2465 | O | HOH | S | 106 | −20.460 | −26.110 | −13.881 | 1.00 | 21.94 | | | O |
| HETATM | 2466 | O | HOH | S | 107 | −4.701 | −27.791 | −8.021 | 1.00 | 32.37 | | | O |
| HETATM | 2467 | O | HOH | S | 108 | −13.168 | −32.064 | 33.597 | 1.00 | 25.14 | | | O |
| HETATM | 2468 | O | HOH | S | 109 | −22.221 | −13.511 | 5.464 | 1.00 | 21.03 | | | O |
| HETATM | 2469 | O | HOH | S | 110 | −24.957 | −29.271 | 7.144 | 1.00 | 22.03 | | | O |
| HETATM | 2470 | O | HOH | S | 111 | −8.225 | −36.233 | 8.923 | 1.00 | 31.19 | | | O |
| HETATM | 2471 | O | HOH | S | 112 | −25.568 | −13.444 | 2.780 | 1.00 | 33.31 | | | O |
| HETATM | 2472 | O | HOH | S | 113 | −22.156 | −26.181 | −3.730 | 1.00 | 25.33 | | | O |
| HETATM | 2473 | O | HOH | S | 114 | −32.266 | −8.986 | 17.820 | 1.00 | 31.11 | | | O |
| HETATM | 2474 | O | HOH | S | 116 | 4.260 | −24.190 | 8.004 | 1.00 | 29.10 | | | O |
| HETATM | 2475 | O | HOH | S | 117 | −28.753 | −8.984 | 20.629 | 1.00 | 23.92 | | | O |
| HETATM | 2476 | O | HOH | S | 118 | −16.996 | −9.380 | 15.014 | 1.00 | 24.79 | | | O |
| HETATM | 2477 | O | HOH | S | 119 | −31.740 | −26.265 | 30.613 | 1.00 | 36.03 | | | O |
| HETATM | 2478 | O | HOH | S | 120 | −23.471 | −30.979 | 17.183 | 1.00 | 40.25 | | | O |
| HETATM | 2479 | O | HOH | S | 121 | −10.517 | −39.145 | 27.260 | 1.00 | 22.57 | | | O |
| HETATM | 2480 | O | HOH | S | 122 | −0.285 | −27.095 | 29.193 | 1.00 | 31.04 | | | O |
| HETATM | 2481 | O | HOH | S | 123 | −29.498 | −20.585 | 1.927 | 1.00 | 31.95 | | | O |
| HETATM | 2482 | O | HOH | S | 124 | −21.756 | −31.098 | 34.133 | 1.00 | 21.20 | | | O |
| HETATM | 2483 | O | HOH | S | 125 | −20.706 | −35.704 | 32.850 | 1.00 | 52.91 | | | O |
| HETATM | 2484 | O | HOH | S | 126 | −3.459 | −32.346 | 31.983 | 1.00 | 35.74 | | | O |
| HETATM | 2485 | O | HOH | S | 127 | −7.962 | −31.166 | 0.651 | 1.00 | 36.65 | | | O |
| HETATM | 2486 | O | HOH | S | 128 | −11.041 | −14.308 | −10.223 | 1.00 | 27.70 | | | O |
| HETATM | 2487 | O | HOH | S | 129 | 2.710 | −9.046 | 3.480 | 1.00 | 27.90 | | | O |
| HETATM | 2488 | O | HOH | S | 130 | −18.450 | −7.550 | 20.468 | 1.00 | 25.93 | | | O |
| HETATM | 2489 | O | HOH | S | 131 | −0.737 | −28.752 | 26.716 | 1.00 | 36.84 | | | O |
| HETATM | 2490 | O | HOH | S | 132 | −21.015 | −10.380 | 9.566 | 1.00 | 33.15 | | | O |
| HETATM | 2491 | O | HOH | S | 133 | −14.549 | −17.305 | 41.265 | 1.00 | 30.61 | | | O |
| HETATM | 2492 | O | HOH | S | 134 | −12.874 | −14.791 | −12.134 | 1.00 | 24.07 | | | O |
| HETATM | 2493 | O | HOH | S | 135 | −23.640 | −33.629 | 14.498 | 1.00 | 28.15 | | | O |
| HETATM | 2494 | O | HOH | S | 137 | −3.196 | −17.249 | −11.223 | 1.00 | 26.70 | | | O |
| HETATM | 2495 | O | HOH | S | 138 | −11.951 | −37.712 | 12.108 | 1.00 | 43.79 | | | O |
| HETATM | 2496 | O | HOH | S | 139 | −28.707 | −28.436 | 11.970 | 1.00 | 24.05 | | | O |
| HETATM | 2497 | O | HOH | S | 140 | −28.811 | −30.544 | 22.744 | 1.00 | 25.91 | | | O |
| HETATM | 2498 | O | HOH | S | 141 | −32.195 | −9.951 | 12.466 | 1.00 | 32.71 | | | O |
| HETATM | 2499 | O | HOH | S | 142 | −5.218 | −8.777 | 9.860 | 1.00 | 36.10 | | | O |
| HETATM | 2500 | O | HOH | S | 143 | −29.647 | −27.732 | 16.776 | 1.00 | 31.21 | | | O |
| HETATM | 2501 | O | HOH | S | 144 | 3.631 | −11.758 | 28.093 | 1.00 | 38.81 | | | O |
| HETATM | 2502 | O | HOH | S | 145 | −10.997 | −5.938 | 37.772 | 1.00 | 34.22 | | | O |
| HETATM | 2503 | O | HOH | S | 146 | −22.550 | −7.889 | 36.301 | 1.00 | 31.59 | | | O |
| HETATM | 2504 | O | HOH | S | 147 | −2.193 | −20.575 | 34.851 | 1.00 | 30.37 | | | O |
| HETATM | 2505 | O | HOH | S | 148 | −25.055 | −31.881 | 12.445 | 1.00 | 35.81 | | | O |
| HETATM | 2506 | O | HOH | S | 149 | 5.972 | −12.854 | 2.827 | 1.00 | 27.17 | | | O |
| HETATM | 2507 | O | HOH | S | 150 | −7.513 | −36.430 | 24.515 | 1.00 | 28.92 | | | O |
| HETATM | 2508 | O | HOH | S | 151 | −25.850 | −16.914 | 0.532 | 1.00 | 24.82 | | | O |
| HETATM | 2509 | O | HOH | S | 152 | −16.196 | −36.096 | 15.495 | 1.00 | 28.80 | | | O |
| HETATM | 2510 | O | HOH | S | 153 | −8.060 | 0.399 | 29.626 | 1.00 | 33.48 | | | O |
| HETATM | 2511 | O | HOH | S | 154 | −28.661 | −17.395 | 4.707 | 1.00 | 31.92 | | | O |
| HETATM | 2512 | O | HOH | S | 156 | −29.746 | −19.750 | 4.525 | 1.00 | 22.45 | | | O |
| HETATM | 2513 | O | HOH | S | 157 | −29.434 | −30.166 | 33.849 | 1.00 | 30.15 | | | O |
| HETATM | 2514 | O | HOH | S | 158 | −31.313 | −12.670 | 30.304 | 1.00 | 28.16 | | | O |
| HETATM | 2515 | O | HOH | S | 159 | −33.005 | −35.360 | 26.684 | 1.00 | 46.31 | | | O |
| HETATM | 2516 | O | HOH | S | 160 | −7.418 | −16.971 | 14.205 | 1.00 | 21.64 | | | O |
| HETATM | 2517 | O | HOH | S | 161 | −2.159 | −10.267 | −0.451 | 1.00 | 25.50 | | | O |
| HETATM | 2518 | O | HOH | S | 163 | −23.462 | −6.685 | 32.849 | 1.00 | 37.35 | | | O |
| HETATM | 2519 | O | HOH | S | 164 | −8.865 | −6.405 | 22.154 | 1.00 | 26.86 | | | O |
| HETATM | 2520 | O | HOH | S | 165 | −5.776 | −36.585 | 20.789 | 1.00 | 35.69 | | | O |
| HETATM | 2521 | O | HOH | S | 166 | −11.950 | −9.681 | 10.412 | 1.00 | 52.04 | | | O |
| HETATM | 2522 | O | HOH | S | 167 | −27.470 | −10.627 | 22.813 | 1.00 | 31.68 | | | O |
| HETATM | 2523 | O | HOH | S | 169 | −30.453 | −10.793 | 10.062 | 1.00 | 31.81 | | | O |
| HETATM | 2524 | O | HOH | S | 170 | −8.893 | −3.592 | 37.874 | 1.00 | 45.39 | | | O |
| HETATM | 2525 | O | HOH | S | 171 | 4.534 | −7.222 | 4.107 | 1.00 | 34.33 | | | O |
| HETATM | 2526 | O | HOH | S | 172 | −12.793 | −36.175 | 10.099 | 1.00 | 30.92 | | | O |
| HETATM | 2527 | O | HOH | S | 173 | −10.811 | −10.681 | 6.610 | 1.00 | 40.30 | | | O |
| HETATM | 2528 | O | HOH | S | 174 | −12.562 | −7.429 | 39.091 | 1.00 | 34.94 | | | O |
| HETATM | 2529 | O | HOH | S | 175 | −19.697 | −8.383 | 14.850 | 1.00 | 29.94 | | | O |
| HETATM | 2530 | O | HOH | S | 176 | 5.940 | −19.869 | 9.889 | 1.00 | 30.45 | | | O |
| HETATM | 2531 | O | HOH | S | 177 | −3.280 | −19.530 | −7.912 | 1.00 | 29.25 | | | O |
| HETATM | 2532 | O | HOH | S | 178 | −22.945 | −7.872 | 16.204 | 1.00 | 37.33 | | | O |
| HETATM | 2533 | O | HOH | S | 179 | −7.022 | −2.597 | 35.760 | 1.00 | 37.46 | | | O |
| HETATM | 2534 | O | HOH | S | 180 | −9.546 | −29.739 | −0.018 | 1.00 | 36.51 | | | O |
| HETATM | 2535 | O | HOH | S | 181 | −5.529 | −2.948 | 27.346 | 1.00 | 35.33 | | | O |
| HETATM | 2536 | O | HOH | S | 182 | 3.065 | −25.869 | 11.515 | 1.00 | 34.32 | | | O |
| HETATM | 2537 | O | HOH | S | 183 | 0.116 | −23.871 | 36.485 | 1.00 | 34.55 | | | O |
| HETATM | 2538 | O | HOH | S | 184 | 4.841 | −23.681 | 12.487 | 1.00 | 36.20 | | | O |
| HETATM | 2539 | O | HOH | S | 185 | −23.376 | −11.894 | 10.150 | 1.00 | 32.92 | | | O |
| HETATM | 2540 | O | HOH | S | 186 | −15.886 | −14.006 | 9.671 | 1.00 | 23.89 | | | O |
| HETATM | 2541 | O | HOH | S | 187 | 5.731 | −20.138 | 0.918 | 1.00 | 25.18 | | | O |
| HETATM | 2542 | O | HOH | S | 188 | −18.599 | −37.259 | 30.615 | 1.00 | 34.73 | | | O |
| HETATM | 2543 | O | HOH | S | 189 | −24.482 | −24.769 | −6.790 | 1.00 | 43.09 | | | O |
| HETATM | 2544 | O | HOH | S | 190 | −10.441 | −14.523 | 13.343 | 1.00 | 35.01 | | | O |
| HETATM | 2545 | O | HOH | S | 191 | −29.115 | −23.334 | 42.124 | 1.00 | 37.02 | | | O |
| HETATM | 2546 | O | HOH | S | 192 | −29.500 | −23.779 | 1.232 | 1.00 | 40.56 | | | O |
| HETATM | 2547 | O | HOH | S | 193 | −28.226 | −30.210 | 38.042 | 1.00 | 41.48 | | | O |
| HETATM | 2548 | O | HOH | S | 194 | −6.796 | −27.383 | −6.655 | 1.00 | 29.03 | | | O |
| HETATM | 2549 | O | HOH | S | 195 | −26.080 | −15.777 | −1.205 | 1.00 | 50.07 | | | O |
| HETATM | 2550 | O | HOH | S | 196 | −27.833 | −10.061 | 24.904 | 1.00 | 42.07 | | | O |
| HETATM | 2551 | O | HOH | S | 197 | −3.459 | −35.453 | 21.016 | 1.00 | 51.21 | | | O |
| HETATM | 2552 | O | HOH | S | 198 | −5.379 | −35.016 | 24.443 | 1.00 | 38.88 | | | O |
| HETATM | 2553 | O | HOH | S | 199 | −9.852 | −32.880 | −3.378 | 1.00 | 39.68 | | | O |
| HETATM | 2554 | O | HOH | S | 200 | −21.530 | −33.763 | 32.275 | 1.00 | 48.39 | | | O |
| HETATM | 2555 | O | HOH | S | 201 | −9.486 | −7.275 | 0.860 | 1.00 | 31.45 | | | O |
| HETATM | 2556 | O | HOH | S | 202 | −26.275 | −10.767 | 35.425 | 1.00 | 42.68 | | | O |
| HETATM | 2557 | O | HOH | S | 203 | −18.589 | −7.519 | 33.367 | 1.00 | 27.20 | | | O |
| HETATM | 2558 | O | HOH | S | 204 | −17.341 | −36.964 | 17.343 | 1.00 | 27.87 | | | O |
| HETATM | 2559 | O | HOH | S | 205 | 2.084 | −25.458 | 17.120 | 1.00 | 27.00 | | | O |
| HETATM | 2560 | O | HOH | S | 206 | 3.851 | −21.058 | 11.401 | 1.00 | 32.64 | | | O |
| HETATM | 2561 | O | HOH | S | 207 | 7.845 | −21.430 | 10.338 | 1.00 | 27.62 | | | O |
| HETATM | 2562 | O | HOH | S | 208 | −5.613 | −18.932 | 22.383 | 1.00 | 34.63 | | | O |
| HETATM | 2563 | O | HOH | S | 210 | −12.003 | −36.514 | 15.521 | 1.00 | 31.33 | | | O |
| HETATM | 2564 | O | HOH | S | 211 | −13.613 | −30.082 | −12.820 | 1.00 | 30.22 | | | O |
| HETATM | 2565 | O | HOH | S | 212 | −24.092 | −24.051 | 39.963 | 1.00 | 27.39 | | | O |
| HETATM | 2566 | O | HOH | S | 213 | −25.216 | −34.320 | 16.616 | 1.00 | 39.96 | | | O |
| HETATM | 2567 | O | HOH | S | 214 | −23.843 | −14.884 | 3.731 | 1.00 | 31.01 | | | O |
| HETATM | 2568 | O | HOH | S | 215 | −21.435 | −7.859 | 33.613 | 1.00 | 29.71 | | | O |
| HETATM | 2569 | O | HOH | S | 216 | −17.346 | −2.961 | 24.469 | 1.00 | 26.91 | | | O |
| HETATM | 2570 | O | HOH | S | 217 | −36.647 | −13.404 | 22.087 | 1.00 | 30.24 | | | O |
| HETATM | 2571 | O | HOH | S | 218 | −9.284 | −24.091 | 40.407 | 1.00 | 38.24 | | | O |
| HETATM | 2572 | O | HOH | S | 219 | 4.383 | −27.008 | 4.367 | 1.00 | 32.13 | | | O |
| HETATM | 2573 | O | HOH | S | 220 | −3.536 | −24.391 | −15.998 | 1.00 | 29.40 | | | O |
| HETATM | 2574 | O | HOH | S | 222 | 2.367 | −13.812 | −1.993 | 1.00 | 29.61 | | | O |
| HETATM | 2575 | O | HOH | S | 223 | −33.886 | −19.588 | 13.827 | 1.00 | 38.69 | | | O |
| HETATM | 2576 | O | HOH | S | 225 | −2.336 | −29.673 | −0.959 | 1.00 | 29.36 | | | O |
| HETATM | 2577 | O | HOH | S | 226 | −10.830 | 2.203 | 33.063 | 1.00 | 43.60 | | | O |
| HETATM | 2578 | O | HOH | S | 227 | −4.074 | −5.849 | 25.841 | 1.00 | 36.60 | | | O |
| HETATM | 2579 | O | HOH | S | 228 | −22.626 | −10.848 | 5.010 | 1.00 | 30.90 | | | O |
| HETATM | 2580 | O | HOH | S | 229 | 2.562 | −7.169 | 19.903 | 1.00 | 47.22 | | | O |
| HETATM | 2581 | O | HOH | S | 230 | −14.998 | −3.423 | 24.921 | 1.00 | 37.21 | | | O |
| HETATM | 2582 | O | HOH | S | 231 | −2.652 | −32.255 | 21.308 | 1.00 | 41.96 | | | O |
| HETATM | 2583 | O | HOH | S | 232 | 6.902 | −11.892 | 7.651 | 1.00 | 27.57 | | | O |
| HETATM | 2584 | O | HOH | S | 233 | −20.308 | −27.366 | −2.451 | 1.00 | 39.06 | | | O |
| HETATM | 2585 | O | HOH | S | 235 | 4.483 | −11.460 | 3.111 | 1.00 | 38.14 | | | O |
| HETATM | 2586 | O | HOH | S | 237 | −7.969 | −26.755 | −9.175 | 1.00 | 31.12 | | | O |
| HETATM | 2587 | O | HOH | S | 238 | −25.252 | −29.452 | 4.590 | 1.00 | 30.91 | | | O |
| HETATM | 2588 | O | HOH | S | 239 | −20.779 | −25.297 | 3.811 | 1.00 | 28.95 | | | O |
| HETATM | 2589 | O | HOH | S | 240 | −12.548 | −17.836 | 42.426 | 1.00 | 37.65 | | | O |
| HETATM | 2590 | O | HOH | S | 241 | −21.037 | −4.730 | 25.423 | 1.00 | 25.30 | | | O |
| HETATM | 2591 | O | HOH | S | 242 | −18.040 | −9.423 | 34.695 | 1.00 | 34.64 | | | O |
| HETATM | 2592 | O | HOH | S | 243 | −31.638 | −22.424 | 38.648 | 1.00 | 32.97 | | | O |
| HETATM | 2593 | O | HOH | S | 244 | 5.668 | −22.531 | 0.378 | 1.00 | 40.48 | | | O |
| HETATM | 2594 | O | HOH | S | 245 | −9.947 | 0.303 | 36.793 | 1.00 | 36.13 | | | O |
| HETATM | 2595 | O | HOH | S | 246 | −13.857 | −16.138 | 10.548 | 1.00 | 31.55 | | | O |
| HETATM | 2596 | O | HOH | S | 247 | −9.097 | −31.140 | 37.124 | 1.00 | 42.40 | | | O |
| HETATM | 2597 | O | HOH | S | 248 | −12.224 | −22.359 | 42.022 | 1.00 | 38.93 | | | O |
| HETATM | 2598 | O | HOH | S | 249 | −9.818 | −10.345 | 13.710 | 1.00 | 38.28 | | | O |
| HETATM | 2599 | O | HOH | S | 250 | −16.581 | −31.411 | 35.954 | 1.00 | 35.72 | | | O |
| HETATM | 2600 | O | HOH | S | 251 | −21.401 | −29.473 | 5.952 | 1.00 | 47.48 | | | O |
| HETATM | 2601 | O | HOH | S | 252 | −1.945 | −9.820 | −9.333 | 1.00 | 38.44 | | | O |
| HETATM | 2602 | O | HOH | S | 253 | −30.015 | −23.200 | 39.877 | 1.00 | 30.60 | | | O |
| HETATM | 2603 | O | HOH | S | 255 | −3.796 | −28.198 | 42.414 | 1.00 | 42.20 | | | O |
| HETATM | 2604 | O | HOH | S | 256 | −5.939 | −25.976 | −11.982 | 1.00 | 35.06 | | | O |
| HETATM | 2605 | O | HOH | S | 257 | 5.453 | −18.688 | −1.225 | 1.00 | 34.80 | | | O |
| HETATM | 2606 | O | HOH | S | 259 | 1.219 | −8.196 | 0.980 | 1.00 | 39.75 | | | O |
| HETATM | 2607 | O | HOH | S | 260 | −0.826 | −9.096 | 29.105 | 1.00 | 41.56 | | | O |
| HETATM | 2608 | O | HOH | S | 262 | −5.387 | −24.214 | −14.530 | 1.00 | 33.12 | | | O |
| HETATM | 2609 | O | HOH | S | 263 | −2.025 | −3.663 | 5.659 | 1.00 | 37.10 | | | O |
| HETATM | 2610 | O | HOH | S | 264 | −12.863 | −30.626 | −2.813 | 1.00 | 26.95 | | | O |
| HETATM | 2611 | O | HOH | S | 266 | −27.043 | −30.780 | 8.504 | 1.00 | 26.68 | | | O |
| HETATM | 2612 | O | HOH | S | 267 | −19.802 | −32.332 | 33.672 | 1.00 | 33.49 | | | O |
| HETATM | 2613 | O | HOH | S | 268 | −30.196 | −24.636 | 15.645 | 1.00 | 33.84 | | | O |
| HETATM | 2614 | O | HOH | S | 269 | −22.456 | −31.549 | 36.548 | 1.00 | 29.51 | | | O |
| HETATM | 2615 | O | HOH | S | 270 | 1.054 | −30.249 | −5.874 | 1.00 | 32.47 | | | O |
| HETATM | 2616 | O | HOH | S | 272 | −21.511 | −6.041 | 1.993 | 1.00 | 44.52 | | | O |
| HETATM | 2617 | O | HOH | S | 273 | 3.613 | −10.343 | 33.440 | 1.00 | 40.42 | | | O |
| HETATM | 2618 | O | HOH | S | 275 | −26.607 | −22.456 | −6.102 | 1.00 | 33.47 | | | O |
| HETATM | 2619 | O | HOH | S | 276 | 5.225 | −7.621 | 12.292 | 1.00 | 45.77 | | | O |
| HETATM | 2620 | O | HOH | S | 278 | 3.034 | −4.987 | 6.079 | 1.00 | 43.47 | | | O |
| HETATM | 2621 | O | HOH | S | 279 | −13.681 | −20.921 | 15.010 | 1.00 | 12.77 | | | O |
| HETATM | 2622 | O | HOH | S | 280 | −25.510 | −13.753 | 7.014 | 1.00 | 35.72 | | | O |
| TER | | | | | | | | | | | | | |
| HETATM | 2623 | O01 | PEG | D | 1 | −16.872 | −29.806 | −3.025 | 1.00 | 48.99 | | A | O |
| HETATM | 2624 | C02 | PEG | D | 1 | −15.628 | −29.263 | −3.189 | 1.00 | 38.71 | | A | C |
| HETATM | 2625 | C03 | PEG | D | 1 | −15.656 | −28.169 | −4.373 | 1.00 | 38.41 | | A | C |
| HETATM | 2626 | O04 | PEG | D | 1 | −14.380 | −27.519 | −4.533 | 1.00 | 38.35 | | A | O |
| HETATM | 2627 | C05 | PEG | D | 1 | −13.277 | −28.340 | −4.770 | 1.00 | 34.73 | | A | C |
| HETATM | 2628 | C06 | PEG | D | 1 | −11.949 | −27.519 | −5.091 | 1.00 | 29.73 | | A | C |
| HETATM | 2629 | O07 | PEG | D | 1 | −10.908 | −27.961 | −4.237 | 1.00 | 40.52 | | A | O |
| HETATM | 2630 | C08 | PEG | D | 1 | −9.764 | −28.601 | −4.795 | 1.00 | 42.14 | | A | C |
| HETATM | 2631 | C09 | PEG | D | 1 | −9.460 | −30.010 | −4.055 | 1.00 | 39.27 | | A | C |
| HETATM | 2632 | O10 | PEG | D | 1 | −9.534 | −29.837 | −2.701 | 1.00 | 40.16 | | A | O |
| HETATM | 2633 | H011 | PEG | D | 1 | −17.050 | −29.987 | −2.214 | 1.00 | 58.79 | | A | H |
| HETATM | 2634 | H021 | PEG | D | 1 | −14.987 | −29.957 | −3.412 | 1.00 | 46.45 | | A | H |
| HETATM | 2635 | H022 | PEG | D | 1 | −15.331 | −28.840 | −2.368 | 1.00 | 46.45 | | A | H |
| HETATM | 2636 | H031 | PEG | D | 1 | −16.343 | −27.517 | −4.163 | 1.00 | 46.10 | | A | H |
| HETATM | 2637 | H032 | PEG | D | 1 | −15.916 | −28.619 | −5.191 | 1.00 | 46.10 | | A | H |
| HETATM | 2638 | H051 | PEG | D | 1 | −13.452 | −28.931 | −5.519 | 1.00 | 41.67 | | A | H |
| HETATM | 2639 | H052 | PEG | D | 1 | −13.103 | −28.892 | −3.992 | 1.00 | 41.67 | | A | H |
| HETATM | 2640 | H061 | PEG | D | 1 | −11.715 | −27.659 | −6.022 | 1.00 | 35.68 | | A | H |
| HETATM | 2641 | H062 | PEG | D | 1 | −12.130 | −26.576 | −4.958 | 1.00 | 35.68 | | A | H |
| HETATM | 2642 | H081 | PEG | D | 1 | −8.979 | −28.039 | −4.709 | 1.00 | 50.56 | | A | H |
| HETATM | 2643 | H082 | PEG | D | 1 | −9.900 | −28.780 | −5.739 | 1.00 | 50.56 | | A | H |
| HETATM | 2644 | H091 | PEG | D | 1 | −10.105 | −30.673 | −4.348 | 1.00 | 47.12 | | A | H |
| HETATM | 2645 | H092 | PEG | D | 1 | −8.577 | −30.322 | −4.308 | 1.00 | 47.12 | | A | H |
| HETATM | 2646 | H101 | PEG | D | 1 | −9.382 | −29.036 | −2.461 | 1.00 | 48.19 | | A | H |
| TER | | | | | | | | | | | | | |
| END | | | | | | | | | | | | | |

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

### Embodiments

The following list of embodiments is intended to complement, rather than displace or supersede, the previous descriptions. All steps in methods described herein, are also relevant for use in any of these methods. All embodiments to methods described herein, also apply for use in such methods.
Embodiment 1. A crystalline composition comprising SEQ ID NO:3, and a ligand, wherein said crystalline composition is characterized with space group p 2 21 21 and has unit cell parameters a=38.155±2Å; b=72.394±2Å; c=103.946±2Å; a=90°; b=90°; g=90°.
Embodiment 2. The crystalline composition according to embodiment 1, wherein the ligand is a compound of Formula (I):

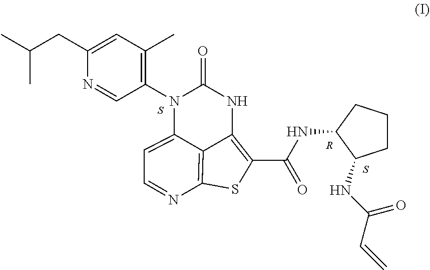

or pharmaceutically acceptable salts, hydrates, polymorphs or solvates thereof.
Embodiment 3. A method for identifying and/or designing a candidate inhibitor of BTK, wherein said method comprises:
generating a three-dimensional structure of a binding site of BTK on a computer, wherein the three dimensional structure coordinates possess the unit cell and space group parameters of the crystalline composition of claim 1,
employing said three dimensional structure to design or select a candidate inhibitor; and
contacting said candidate inhibitor with human BTK and measuring the ability of said candidate inhibitor to bind to BTK.
Embodiment 4. The method of embodiment 3, wherein the candidate inhibitor makes a direct covalent bond with Cys 481.
Embodiment 5. The method of embodiment 3, wherein the candidate inhibitor makes a hydrogen bond with Lys 430.
Embodiment 6. The method of embodiment 3, wherein the candidate inhibitor makes a hydrogen bond with Met 477.
Embodiment 7. A method for identifying and/or designing a candidate inhibitor using a human BTK crystal comprising a human BTK protein, wherein said method comprises:
a) preparing the crystalline composition of claim 2;
b) soaking another candidate inhibitor into the crystalline composition, displacing the compound of Formula (I) to form an inhibitor-crystal complex;
c) determining the three-dimensional structure coordinates of the inhibitor-crystal complex prepared in step b);
d) using the structure coordinates from step c) to design or select a candidate inhibitor; and
e) contacting said candidate inhibitor with human BTK and measuring the ability of said candidate inhibitor to bind to BTK.

Embodiment 8, A method of designing a compound or complex that interacts with a binding pocket or domain selected from the group consisting of:
(i) human BTK amino acid residues Leu408, Gly409, Thr410, Gly411, Val416, Ala428, Lys430, Asn439, Met449, Leu452, Val458, Ile472, Thr474, Glu475, Tyr476, Met477, Gly480, Cys481, Asn 484, Arg525, Leu528, Ser538, Asp539, and Phe540 according to Table 2; and
(ii) human BTK amino acid residues according to Table 2;
comprising the steps of:
(a) producing a crystal of BTK in complex with a compound of Formula (I):

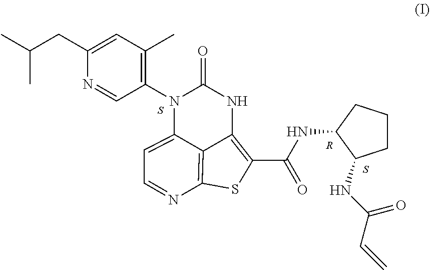

or pharmaceutically acceptable salts, hydrates, polymorphs or solvates thereof, wherein said BTK comprises SEQ ID NO: 3, wherein said crystal is characterized with space group p 2 21 21 and has unit cell parameters a=38.155±2Å; b=72.394±2Å; c=103.946±2Å; a=90°; b=90°; g=90°;
(b) providing the structure coordinates of said binding pocket or domain of the crystal of step (a), according to Table 2, on a computer capable of generating three-dimensional structural information from said structure coordinates, wherein the root mean square deviation of the backbone atoms is not greater than about 2.5Å;
(c) using the computer to dock a first chemical entity in part of the binding pocket or domain;
(d) docking at least a second chemical entity in another part of the binding pocket or domain;
(e) quantifying the association between the first or second chemical entity and part of the binding pocket or domain;
(f) optionally repeating steps (c) to (e) with one or more additional chemical entities, selecting the additional chemical entities based on said quantified association of all the first, second and one or more additional chemical entities;
(g) optionally, visually inspecting the relationship of the first, second and one or more additional chemical entity to each other in relation to the binding pocket or domain on a computer screen using the three-dimensional graphical representation of the binding pocket or domain and said first, second and one or more additional chemical entities; and
(h) assembling the first, second and one or more additional chemical entity into a compound or complex that interacts with said binding pocket or domain by model building.

Embodiment 9. A method for identifying a candidate inhibitor that interacts with a binding site of a BTK protein, comprising the steps of:
(a) producing a crystal of BTK in complex with a compound of Formula (I):

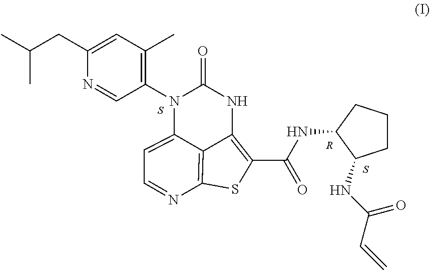

or pharmaceutically acceptable salts, hydrates, polymorphs or solvates thereof, wherein said BTK comprises SEQ ID NO: 3. wherein the crystal is characterized with space group p 2 21 21 and has unit cell parameters a=38.155±2Å; b=72.394±2Å; c=103.946±2Å; a=90°; b=90°; g=90°;
(b) obtaining the stnicture coordinates of amino acids of the crystal of step (a) according to Table 2;
(c) generating a three-dimensional model of said BTK protein using the stnicture coordinates of the amino acids generated in step (b), wherein the root mean square deviation from backbone atoms is not more than ±2.0 Å;
(d) determining a binding site of said human BTK protein from said three-dimensional model; and
(e) performing computer fitting analysis to identify the candidate inhibitor which interacts with said binding site.
Embodiment 10, The method according to embodiment 9, further comprising the step of:
(f) contacting the identified candidate inhibitor with said BTK protein in order to determine the effect of the inhibitor on BACK activity.
Embodiment 11, The method according to embodiment 9, wherein the binding site of said BTK protein determined in step (d) comprises the structure coordinates, according to Table 2, of BTK amino acid residues Leu408, Gly409, Thr410, Gly411, Val416, Ala428, Lys430, Asn439, Met449, Leu452, Val458, Ile472, Thr474, Glu475, Tyr476, Met477, Gly480, Cys481, Asn 484, Arg525, Leu528, Ser538,Asp539, and Phe540, wherein the root mean square deviation is not more than ±2.0 Å.
Embodiment 12. A method of using a crystal comprising a BTK binding pocket or domain in an inhibitor screening assay comprising the steps of:
(a) producing a crystal of BTK in complex with a compound of Formula (I):

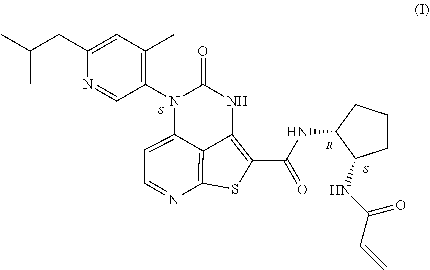

or pharmaceutically acceptable salts, hydrates, polymorphs or solvates thereof, wherein said BTK comprises SEQ ID NO: 3, wherein said crystal is characterized with space group p 2 21 21 and has unit cell parameters a=38.155±2Å; b=72.394±2Å; c=103.946±2Å; a=90°; b=90°; g=90°;
(b) providing the structure coordinates of said binding pocket or domain of the crystal of step (a), according to Table 2, on a computer capable of generating three-dimensional structural information from said structure coordinates;
(c) selecting a potential inhibitor by performing rational drug design with a three-dimensional structure determined for the crystal, wherein said selecting is performed in conjunction with computer modeling;
(d) contacting the potential inhibitor with the kinase; and
(e) detecting the ability of the potential inhibitor for inhibiting the kinase's enzymatic activity.
Embodiment 13. A method of designing a compound or complex that interacts with a BTK binding pocket or domain comprising the steps of:
(a) producing a crystal of human BTK in complex with a compound of Formula (I):

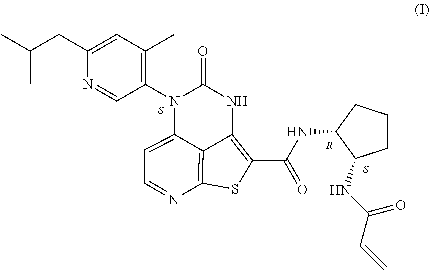

or pharmaceutically acceptable salts, hydrates, polymorphs or solvates thereof, wherein said BTK comprises SEQ ID NO: 3, wherein said crystal is characterized with space group space group p 2 21 21 and has unit cell parameters a=38.155±2Å; b=72.394±2Å; c=103.946±2Å; a=90°; b=90°; g=90°;
(b) providing the structure coordinates of said binding pocket or domain of the crystal of step (a), according to Table 2, on a computer capable of generating three-dimensional structural information from said structure coordinates;
(c) using the computer to dock a first chemical entity in part of the binding pocket or domain;
(d) docking at least a second chemical entity in another part of the binding pocket or domain;
(e) quantifying the association between the first or second chemical entity and part of the binding pocket or domain;
(f) repeating steps (c) to (e) with at least one additional chemical entity, selecting a first, second and at least one additional chemical entity based on said quantified association of all of said first, second and at least one additional chemical entity;
(g) optionally, visually inspecting the relationship of the first, second and at least one additional chemical entity to each other in relation to the binding pocket or domain on a computer screen using the three-dimensional graphical representation of the binding pocket or domain and said first, second and at least one additional chemical entity; and
(h) assembling the first, second and at least one additional chemical entity into a compound or complex that interacts with said binding pocket or domain by model building.
Embodiment 14. A method for identifying a candidate inhibitor that interacts with a binding site of a BTK protein comprising the steps of:
(a) producing a crystal of BTK in complex with a compound of Formula (I):

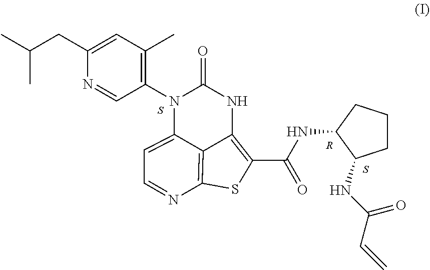

or pharmaceutically acceptable salts, hydrates, polymorphs or solvates thereof, wherein said BTK comprises SEQ ID NO: 3, wherein said crystal is characterized with space group p 2 21 21 and has unit cell parameters a=38.155±2Å; b=72.394±2Å; c=103.946±2Å; a=90°; b=90°; g=90°;
(b) determining the three-dimensional structure coordinates of BTK using the crystal. in step (a);
(c) using the structure coordinates from step (b) to generate a three-dimensional structure of the crystallized BTK protein produced in step (a);
(d) employing said three-dimensional structure to design or select said candidate inhibitor;
(e) synthesizing said candidate inhibitor; and
(f) contacting said candidate inhibitor with human BTK protein to determine the ability of said candidate inhibitor to bind to human BTK.
Embodiment 15. A method for identifying a candidate inhibitor that interacts with a binding site of a human BTK protein comprising the steps of:
(a) producing a crystal of BTK in complex with a compound of Formula (I):

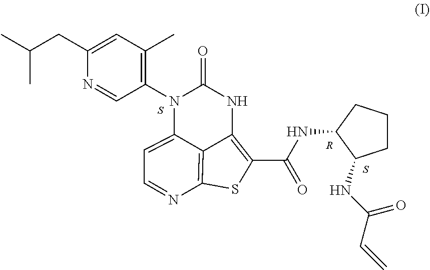

or pharmaceutically acceptable salts, hydrates, polymorphs or solvates thereof, wherein said BTK comprises SEQ ID NO: 3, wherein said crystal is characterized with space group p 2 21 21 and has unit cell parameters a=38.155±2Å; b=72.394±2Å; c=103.946±2Å; a=90°; b=90°; g=90°;
(b) determining the three-dimensional structure coordinates of13TK using the crystal in step (a);
(c) using the structure coordinates from step (b) to generate a three-dimensional structure of a molecular complex comprising a binding site of amino acid residues Leu408, Gly409, Thr410, Gly411, Val416, Ala428, Lys430, Asn439, Met449, Leu452, Val458, Ile472, Thr474, Glu475, Tyr476 Met477, Gly480, Cys481, Asn 484, Arg525, Leu528, Ser538, Asp539, and Phe540 according to Table 2, wherein the root mean square deviation of the backbone atoms is not greater than about 2.5 Å;
(d) employing said three-dimensional structure to design or select said candidate inhibitor;
(e) synthesizing said candidate inhibitor; and
(f) contacting said candidate inhibitor with BTK to determine the ability of said candidate inhibitor to bind to BTK.

Sequence listing
NUMBER OF SEQ ID NOS: 3
LENGTH: 1500
TYPE: DNA
ORGANISM: Homo sapiens
SEQUENCE: 1
SEQ ID NO 1
atgtccccta tactaggtta ttggaaaatt aagggccttg tgcaacccac tcgacttctt
ttggaatatc ttgaagaaaa atatgaagag catttgtatg agcgcgatga aggtgataaa
tggcgaaaca aaaagtttga attgggtttg gagtttccca atcttcctta ttatattgat
ggtgatgtta aattaacaca gtctatggcc atcatacgtt atatagctga caagcacaac
atgttgggtg gttgtccaaa agagcgtgca gagatttcaa tgcttgaagg agcggttttg
gatattagat acggtgtttc gagaattgca tatagtaaag actttgaaac tctcaaagtt
gattttctta gcaagctacc tgaaatgctg aaaatgttcg aagatcgttt atgtcataaa
acatatttaa atggtgatca tgtaacccat cctgacttca tgttgtatga cgctcttgat
gttgttttat acatggaccc aatgtgcctg gatgcgttcc caaaattagt ttgttttaaa
aaacgtattg aagctatccc acaaattgat aagtacttga aatccagcaa gtatatagca
tggcctttgc agggctggca agccacgttt ggtggtggcg accatcctcc aaaatcggat
acgaccgaaa acctgtattt tcagggcctg ggatacggat catgggaaat tgatccaaag
gacctgacct tcttgaagga gctggggact ggacaatttg gggtagtgaa gtatgggaaa
tggagaggcc agtacgacgt ggccatcaag atgatcaaag aaggctccat gtctgaagat
gaattcattg aagaagccaa agtcatgatg aatctttccc atgagaagct ggtgcagttg
tatggcgtct gcaccaagca gcgccccatc ttcatcatca ctgagtacat ggccaatggc
tgcctcctga actacctgag ggagatgcgc caccgcttcc agactcagca gctgctagag
atgtgcaagg atgtctgtga agccatggaa tacctggagt caaagcagtt ccttcaccga
gacctggcag ctcgaaactg tttggtaaac gatcaaggag ttgttaaagt atctgatttc
ggcctgtcca ggtatgtcct ggatgatgaa tacacaagct cagtaggctc caaatttcca
gtccggtggt ccccaccgga agtcctgatg tatagcaagt tcagcagcaa atctgacatt
tgggcttttg gggttttgat gtgggaaatt tactccctgg ggaagatgcc atatgagaga
tttactaaca gtgagactgc tgaacacatt gcccaaggcc tacgtctcta caggcctcat
ctggcttcag agaaggtata taccatcatg tacagttgtt ggcatgagaa agcagatgag
cgtcccactt tcaaaattct tctgagcaat attctagatg tcatggatga agaatcctga
LENGTH: 228
TYPE: PRT
ORGANISM: Homo sapiens
SEQUENCE: 2
SEQ ID NO 2
MSPILGYWKI KGLVQPTRLL LEYLEEKYEE HLYERDEGDK WRNKKFELGL EFPNLPYYID
GDVKLTQSMA IIRYIADKHN MLGGCPKERA EISMLEGAVL DIRYGVSRIA YSKDFETLKV
DFLSKLPEML KMFEDRLCHK TYLNGDHVTH PDFMLYDALD VVLYMDPMCL DAFPKLVCFK
KRIEAIPQID KYLKSSKYIA WPLQGWQATF GGGDHPPKSD TTENLYFQ
LENGTH: 271
TYPE: PRT
ORGANISM: Homo sapiens
SEQUENCE: 3
SEQ ID NO 3
GLGYGSWEID PKDLTFLKEL GTGQFGVVKY GKWRGQYDVA IKMIKEGSMS EDEFIEEAKV
MMNLSHEKLV QLYGVCTKQR PIFIITEYMA NGCLLNYLRE MRHRFQTQQL LEMCKDVCEA
MEYLESKQFL HRDLAARNCL VNDQGVVKVS DFGLSRYVLD DEYTSSVGSK FPVRWSPPEV
LMYSKFSSKS DIWAFGVLMW EIYSLGKMPY ERFTNSETAE HIAQGLRLYR PHLASEKVYT
IMYSCWHEKA DERPTFKILL SNILDVMDEE S

We claim:

1. A crystalline composition comprising SEQ ID NO:3, and a ligand, wherein said crystalline composition is characterized with space group p 2 21 21 and has unit cell parameters a=38.155 2Å; b=72.394±2Å; c=103.946±2Å; a=90°; b=90°; g=90°.

2. The crystalline composition according to claim 1, wherein the ligand is a compound of Formula (I): 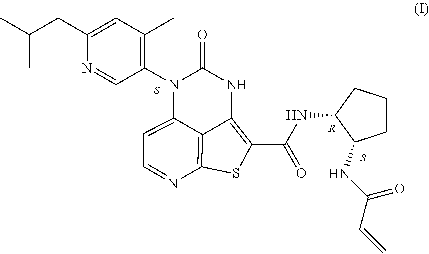
or pharmaceutically acceptable salts, hydrates, polymorphs or solvates thereof

3. A method for identifying and/or designing a candidate inhibitor of BTK, wherein said method comprises:
generating a three-dimensional structure of a binding site of BTK on a computer, wherein the three dimensional structure coordinates possess the unit cell and space group parameters of the crystalline composition of claim 1,
employing said three dimensional structure to design or select a candidate inhibitor; and
contacting said candidate inhibitor with human BTK and measuring the ability of said candidate inhibitor to bind to BTK.

4. The method of claim 3, wherein the candidate inhibitor makes a direct covalent bond with Cys 481.

5. The method of claim 3, wherein the candidate inhibitor makes a hydrogen bond with Lys 430.

6. The method of claim 3, wherein the candidate inhibitor makes a hydrogen bond with Met 477.

7. A method for identifying and/or designing a candidate inhibitor using a human BTK crystal comprising a human BTK protein, wherein said method comprises:
a) preparing the crystalline composition of claim 2;
b)soaking another candidate inhibitor into the crystalline composition, displacing the compound of Formula (I) to form an inhibitor-crystal complex;
c) determining the three-dimensional structure coordinates of the inhibitor-crystal complex prepared in step b);d) using the structure coordinates from step c) to design or select a candidate inhibitor; and
e) contacting said candidate inhibitor with human BTK and measuring the ability of said candidate inhibitor to bind to BTK.

8. A method of designing a compound or complex that interacts with binding pocket or domain selected from the group consisting of:
(i) human BTK amino acid residues Leu408, Gly409, Thr410, Gly411, Val416, Ila428, Lys430, Asn439, Met449, Leu452, Val458, Ile472, Thr474, Glu475, Tyr476, Met477, Gly480, Cys481, Asn 484, Arg525, Leu528, Ser538, Asp539, and Phe540 according to Table 2; and
(ii) human BTK amino acid residues according to Table 2;
comprising the steps of:
(a) producing a crystal of BTK in complex with a compound of Formula (I): 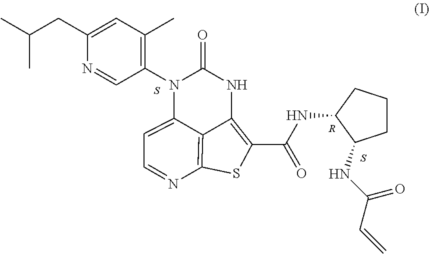
or pharmaceutically acceptable salts, hydrates, polymorphs or solvates thereof, wherein said BTK comprises SEQ ID NO: 3, wherein said crystal is characterized with space group p 2 21 21 and has unit cell parameters a=38.155±2Å; b=72.394±2Å; c=103.946±2Å; a=90°; b=90°; g=90°;
(b) providing the structure coordinates of said binding pocket or domain of the crystal of step (a), according to Table 2, on a computer capable of generating three-dimensional structural information from said structure coordinates, wherein the root mean square deviation of the backbone atoms is not greater than about 2.5 Å;
(c) using the computer to dock a first chemical entity in part of the binding pocket or domain;
(d) docking at least a second chemical entity in another part of the binding pocket or domain;
(e) quantifying the association between the first or second chemical entity and part of the binding pocket or domain;
(f) optionally repeating steps (c) to (e) with one or more additional chemical entities, selecting the additional chemical entities based on said quantified association of all the first, second and one or more additional chemical entities;
(g) optionally, visually inspecting the relationship of the first, second and one or more additional chemical entity to each other in relation to the binding pocket or domain on a computer screen using the three-dimensional graphical representation of the binding pocket or domain and said first, second and one or more additional chemical entities; and
(h) assembling the first, second and one or more additional chemical entity into a compound or complex that interacts with said binding pocket or domain by model building.

9. A method for identifying a candidate inhibitor that interacts with a binding site of a BTK protein, comprising the steps of:
(a) producing a crystal of BTK in complex with a compound of Formula (I): 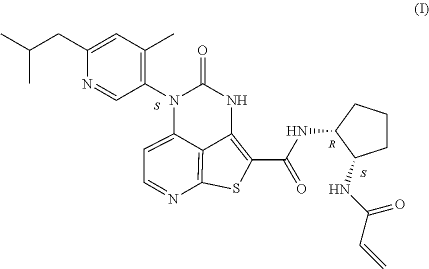
or pharmaceutically acceptable salts, hydrates, polymorphs or solvates thereof, wherein said BTK comprises SEQ ID NO: 3, wherein the crystal is characterized with space group p 2 21 21 and has unit cell parameters a=38.155±2Å; b=72.394±2Å; c=103.946±2Å; a=90°; b=90°; g=90°;
(b) obtaining the structure coordinates of amino acids of the crystal of step (a) according to Table 2;
(c) generating a three-dimensional model of said BTK protein using the structure coordinates of the amino acids generated in step (b), wherein the root mean square deviation from backbone atoms is not more than +2.0 Å;
(d) determining a binding site of said human BTK protein from said three-dimensional model; and
(e) performing computer fitting analysis to identify the candidate inhibitor which interacts with said binding site.

10. The method according to claim 9, further comprising the step of:
(f) contacting the identified candidate inhibitor with said BTK protein in order to determine the effect of the inhibitor on BTK activity.

11. The method according to claim 9, wherein the binding site of said BTK protein determined in step (d) comprises the structure coordinates, according to Table 2, of BTK amino acid residues Leu408, Gly409, Thr410, Gly411, Val416, Ala428, Lys430, Asn439, Met449, Leu452, Val458, Ile472, Thr474, Glu475, Tyr476, Met477, Gly480, Cys481, Asn 484, Arg525, Leu528, Ser538, Asp539, and Phe540, wherein the root mean square deviation is not more than +2.0 Å.

12. A method of using a crystal comprising a BTK binding pocket or domain in an inhibitor screening assay comprising the steps of:
(a) producing a crystal of BTK in complex with a compound of Formula (I): 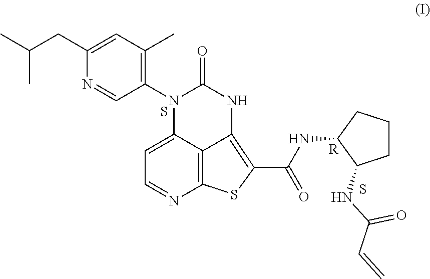
or pharmaceutically acceptable salts, hydrates, polymorphs or solvates thereof, wherein said BTK comprises SEQ ID NO: 3, wherein said crystal is characterized with space group p 2 21 21 and has unit cell parameters a=38.155±2Å; b=72.394±2Å; c=103.946±2Å; a=90°; b=90°; g=90°;
(b) providing the structure coordinates of said binding pocket or domain of the crystal of step (a), according to Table 2, on a computer capable of generating three-dimensional structural information from said structure coordinates;
(c) selecting a potential inhibitor by performing rational drug design with a three-dimensional structure determined for the crystal, wherein said selecting is performed in conjunction with computer modeling;
(d) contacting the potential inhibitor with the kinase; and
(e) detecting the ability of the potential inhibitor for inhibiting the kinase's enzymatic activity.

13. A method of designing a compound or complex that interacts with a BTK binding pocket or domain comprising the steps of:
(a) producing a crystal of human BTK in complex with a compound of Formula (I): 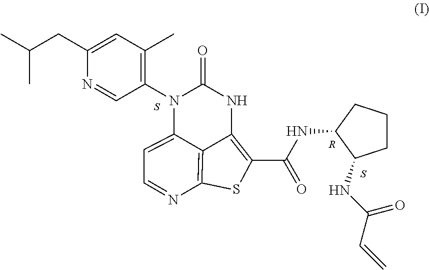
or pharmaceutically acceptable salts, hydrates, polymorphs or solvates thereof, wherein said BTK comprises SEQ ID NO: 3, wherein said crystal is characterized with space group space group p 2 21 21 and has unit cell parameters a=38.155±2Å; b=72.394±2Å; c=103.946±2Å;
a=90°; b=90°; g=90°;
(b) providing the structure coordinates of said binding pocket or domain of the crystal of step (a), according to Table 2, on a computer capable of generating three-dimensional structural information from said structure coordinates;
(c) using the computer to dock a first chemical entity in part of the binding pocket or domain;
(d) docking at least a second chemical entity in another part of the binding pocket or domain;
(e) quantifying the association between the first or second chemical entity and part of the binding pocket or domain;
(f) repeating steps (c) to (e) with at least one additional chemical entity, selecting a first, second and at least one additional chemical entity based on said quantified association of all of said first, second and at least one additional chemical entity;
(g) optionally, visually inspecting the relationship of the first, second and at least one additional chemical entity to each other in relation to the binding pocket or domain on a computer screen using the three-dimensional graphical representation of the binding pocket or domain and said first, second and at least one additional chemical entity; and
(h) assembling the first, second and at least one additional chemical entity into a compound or complex that interacts with said binding pocket or domain by model building.

14. A method for identifying a candidate inhibitor that interacts with a binding site of a BTK protein comprising the steps of:
(a) producing a crystal of BTK in complex with a compound of Formula (I): 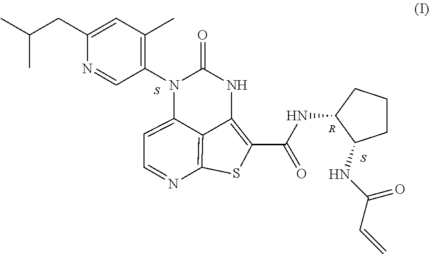
or pharmaceutically acceptable salts, hydrates, polymorphs or solvates thereof, wherein said BTK comprises SEQ ID NO: 3, wherein said crystal is characterized with space group p 2 21 21 and has unit cell parameters a=38.155±2Å; b=72.394±2Å; c=103.946±2Å; a=90°; b=90°; g=90°;
(b) determining the three-dimensional structure coordinates of BTK using the crystal in step (a);
(c) using the structure coordinates from step (b) to generate a three-dimensional structure of the crystallized BTK protein produced in step (a);
(d) employing said three-dimensional structure to design or select said candidate inhibitor;
(e) synthesizing said candidate inhibitor; and
(t) contacting said candidate inhibitor with human BTK protein to determine the ability of said candidate inhibitor to bind to human BTK.

15. A method for identifying a candidate inhibitor that interacts with a binding site of a human BTK protein comprising the steps of:
(a) producing a crystal of BTK in complex with a compound of Formula (I): 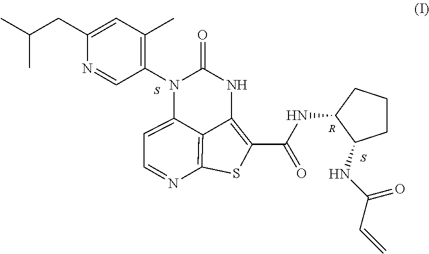
or pharmaceutically acceptable salts, hydrates, polymorphs or solvates thereof, wherein said BTK comprises SEQ ID NO: 3, wherein said crystal is characterized with space group p 2 21 21 and has unit cell parameters a=38.155±2Å; b=72.394±2Å; c=103.946±2Å; a=90°; b=90°; g=90°;
(b) determining the three-dimensional structure coordinates of BTK using the crystal in step (a);
(c) using the structure coordinates from step (b) to generate a three-dimensional structure of a molecular complex comprising a binding site of amino acid residues Leu408, Gly409, Thr410, Gly411, Val416, Ala428, Lys430, Asn439, Met449, Leu452, Val458, Ile472, Thr474, Glu475, Tyr476, Met477, Gly480, Cys481, Asn 484, Arg525, Leu528, Ser538, Asp539, and Phe540 according to Table 2, wherein the root mean square deviation of the backbone atoms is not greater than about 2.5 Å;
(d) employing said three-dimensional structure to design or select said candidate inhibitor;
(e) synthesizing said candidate inhibitor; and
(f) contacting said candidate inhibitor with BTK to determine the ability of said candidate inhibitor to bind to BTK.
